(19) United States
(12) Patent Application Publication
(10) Pub. No.: US 2025/0064933 A1
(43) Pub. Date: Feb. 27, 2025

(54) CHIMERIC ANTIGEN RECEPTORS COMPRISING A PDZ BINDING MOTIF

(71) Applicant: St. Jude Children's Research Hospital, Inc., Memphis, TN (US)

(72) Inventors: Stephen GOTTSCHALK, Germantown, TN (US); Peter CHOCKLEY, Memphis, TN (US)

(73) Assignee: St. Jude Children's Research Hospital, Inc., Memphis, TN (US)

(21) Appl. No.: 18/724,820

(22) PCT Filed: Dec. 29, 2022
(86) PCT No.: PCT/US2022/082516

§ 371 Date: Jun. 27, 2024

Related U.S. Application Data
(60) Provisional application No. 63/294,873, filed on Dec. 30, 2021.

(51) Int. Cl. A61K 39/00; A61P 35/00; C07K 14/725; C07K 16/30

(52) U.S. Cl. CPC A61K 39/4644; A61K 39/4611; A61K 39/4613; A61K 39/4631; A61P 35/00; C07K 14/7051; C07K 16/30; C07K 2317/622; C07K 2319/03

(57) ABSTRACT

The present application provides chimeric antigen receptors (CARs) comprising an anchoring domain, such as a PDZ binding motif, which binds to a cell polarity protein. Also provided are poly nucleotides encoding the CARs, vectors, and cell compositions comprising the same. Pharmaceutical compositions comprising the polypeptides, polynucleotides, vectors, or cells of the present disclosure, and their uses in treating a disease in a subject are also provided.

### CROSS-REFERENCE TO RELATED APPLICATIONS

This application claims priority to U.S. Provisional Application No. 63/294,873, filed Dec. 30, 2021, the disclosure of which is herein incorporated by reference in its entirety.

### FIELD OF THE INVENTION

The present application relates to chimeric antigen receptors (CARs) comprising an anchoring domain, such as a PDZ binding motif, which binds to a cell polarity protein.

### BACKGROUND

Chimeric antigen receptor (CAR) technologies have been clinically implemented for the treatment of hematological malignancies; however, solid tumors remain resilient to CAR therapeutics¹⁻³. CAR technology has produced exceptionally powerful and effective signaling in both Natural Killer (NK) and T cells⁷. However, CAR:Antigen complexes form disordered synapses⁸, which do not consist of bona fide central, peripheral, or distal supramolecular activation complexes (SMACs) in contrast to canonical T cell Receptor to Human Leukocyte Antigen (TCR:HLA) synapses⁸. These organized SMACs allow for a lower threshold for antigen recognition by at least 3 logs⁹. Further, the additional signaling molecules recruited to the synapse increase the efficiency of signaling and exclude inhibitory phosphatases¹⁰. In contrast, the disjointed CAR synapse is a punctate structure with islands of CAR:Antigen complexes^{9,11}. These islands are, putatively, open to dephosphorylation and thus require a larger number of interactions to initiate downstream signaling and activate T cells.

### SUMMARY OF THE INVENTION

As specified in the Background section above, there is a great need in the art for synaptic modulation to an increasingly ordered state, e.g., via synapse tuning, to enhance efficacy and effectiveness of CARs. The present application addresses these and other needs.

In one aspect, the present invention provides a polynucleotide encoding a chimeric antigen receptor (CAR) which may comprise: a) an extracellular domain; b) a transmembrane domain; and, c) cytoplasmic domain comprising a signaling domain and an anchoring domain which binds to a cell polarity protein.

In some embodiments, the cell polarity protein may comprise a Postsynaptic density-95, Discs large, and Zona occludens 1 (PDZ) domain. In some embodiments, the anchoring domain may comprise a PDZ binding motif (PDZbm).

In some embodiments, the PDZbm may bind to a PDZ domain-containing protein selected from AAG12, AHNAK, AHNAK2, AIP1, ALP, APBA1, APBA2, APBA3, ARHGAP21, ARHGAP23, ARHGEF11, ARHGEF12, CARD10, CARD11, CARD14, CASK, CLP-36, CNKSR2, CNKSR3, CRTAM, DFNB31, DLG1, DLG2, DLG3, DLG4, DLG5, DVL1, DVL1L1, DVL2, DVL3, ERBB2IP, FRMPD1, FRMPD2, FRMPD2L1, FRMPD3, FRMPD4, GIPC1, GIPC2, GIPC3, GOPC, GRASP, GRIP1, GRIP2, HTRA1, HTRA2, HTRA3, HTRA4, IL16, INADL, KIAA1849, LDB3, LIMK1, LIMK2, LIN7A, LIN7B, LIN7C, LMO7, LNX1, LNX2, LRRC7, MAGI1, MAGI2, MAGI3, MAGIX, MAST1, MAST2, MAST3, MAST4, MCSP, MLLT4, MPDZ, MPP1, MPP2, MPP3, MPP4, MPP5, MPP6, MPP7, MYO18A, NHERF1, NOS1, PARD3, PARD6A, PARD6B, PARD6G, PDLIM1, PDLIM2, PDLIM3, PDLIM4, PDLIM5, PDLIM7, PDZD11, PDZD2, PDZD3, PDZD4, PDZD5A, PDZD7, PDZD8, PDZK1, PDZRN3, PDZRN4, PICK1, PPP1R9A, PPP1R9B, PREX1, PRX, PSCDBP, PTPN13, PTPN3, PTPN4, RAPGEF2, RGS12, RGS3, RHPN1, RIL, RIMS1, RIMS2, SCN5A, SCRIB (or Scribble), SDCBP, SDCBP2, SHANK1, SHANK2, SHANK3, SHROOM2, SHROOM3, SHROOM4, SIPA1, SIPA1L1, SIPA1L2, SIPA1L3, SLC9A3R1, SLC9A3R2, SNTA1, SNTB1, SNTB2, SNTG1, SNTG2, SNX27, SPAL2, STXBP4, SYNJ2BP, SYNPO2, SYNPO2L, TAX1BP3, TIAM1, TIAM2, TJP1, TJP2, TJP3, TRPC4, TRPC5, USH1C, and WHEN. In one embodiment, the PDZbm may bind to SCRIB (or Scribble).

In some embodiments, the PDZbm may be derived from any of the 16 classes of PDZ binding proteins as defined by the following C-terminal motifs: 1a (φ[K/R]XSDV); 1b (Ω[R/K]ET[S/T/R/K]φ); 1c (φφETXL); 1d (ETXV); 1e (TWΨ); 1f (≠ΩTWΨ); 1g (φφφ[T/S][T/S]ΩΨ); 1h (φφ[D/E][T/S]WΨ); 2a (FDΩΩC); 2b (WXΩFDV); 2c (WΩφDΨ); 2d (φφX[E/D]φφφ); 2e (φφφφ); 2f ([D/E]φΩφ); 3a (WΩ[S/T]DWΨ); 4a (ΩφGWF); φ, hydrophobic (V, I, L, F, W, Y, M); Ω, aromatic (F, W, and Y); Ψ, aliphatic (V, I, L, and M); and X, nonspecific. In one embodiment, the PDZbm may be derived from Cytotoxic and Regulatory T cell Associated Molecule (CRTAM).

In some embodiments, the PDZbm may comprise the amino acid sequence of ESIV (SEQ ID NO: 1). In some embodiments, the PDZbm may be encoded by the nucleotide sequence of gagagcatcgtg (SEQ ID NO: 2).

In some embodiments, the PDZbm may comprise the amino acid sequence of HPMRCMNYITKLYSEAKTKRKENVQHSKLEEKHIQVPESIV (SEQ ID NO: 3). In some embodiments, the PDZbm may be encoded by the nucleotide sequence of caccccatgoggtgcatgaactacatcaccaagctgtactccgaggccaagaccaageggaaagagaacgtccagcacagcaagc tggaagagaagcacattcaggtgcccgagagcatcgtgtga (SEQ ID NO: 4).

In some embodiments, the anchoring domain may be located at the C-terminal position of the CAR.

In some embodiments, the extracellular domain may comprise an antigen-binding moiety. In some embodiments, the antigen-binding moiety may be an antibody or antibody fragment. In some embodiments, the antigen-binding moiety may be a single chain variable fragment (scFv). In some embodiments, the antigen-binding moiety may be a ligand or peptide sequence. In some embodiments, the antigen-binding moiety may bind to a tumor antigen, antigen of extracellular matrix, antigen present on cells within the tumor microenvironment, tissue-specific antigen, autoimmune antigen or infectious antigen. In some embodiments, the antigen-binding moiety binds EphA2 or B7-H3.

In some embodiments, the transmembrane domain may be derived from CD8α, CD28, CD8, CD4, CD3ζ, CD40, CD134 (OX-40), NKG2A/C/D/E, or CD7. In some embodiments, the transmembrane domain may be derived from CD28.

In some embodiments, the extracellular domain may further comprise a hinge domain between the antigen-binding moiety and the transmembrane domain. In some embodiments, the hinge domain may be derived from CD8α stalk, CD28 or an IgG. In some embodiments, the hinge domain may be a short hinge domain derived from IgG1, IgG2, IgG3, or IgG4.

In some embodiments, the signaling domain may be derived from CD3ζ, DAP10, DAP12, Fc epsilon receptor I γ chain (FCER1G), CD3δ, CD3ε, CD3γ, CD226, NKG2D, or CD79A. In some embodiments, the signaling domain may be derived from CD3ζ.

In some embodiments, the cytoplasmic domain may further comprise one or more costimulatory domains. In some embodiments, the one or more costimulatory domains may be derived from CD28, 4-1BB, CD27, CD40, CD134, CD226, CD79A, ICOS, or MyD88, or any combination thereof. In some embodiments, the cytoplasmic domain may comprise a CD28 costimulatory domain.

In some embodiments, the extracellular target-binding domain may further comprise a leader sequence. In some embodiments, the leader sequence may be derived from CD8α or human immunoglobulin heavy chain variable region.

In some embodiments, the polynucleotide of the present disclosure may be a DNA molecule.

In some embodiments, the polynucleotide of the present disclosure may be an RNA molecule.

In some embodiments, the polynucleotide may be expressed in an inducible fashion, achieved with an inducible promoter, an inducible expression system, an artificial signaling circuit, and/or drug induced splicing.

In some embodiments, the promoter may be a T cell-specific promoter or an NK cell-specific promoter.

In some embodiments, the polynucleotide of the present disclosure may further comprise one or more additional nucleotide sequences encoding one or more additional polypeptide sequences. In some embodiments, the one or more additional polypeptide sequences may be selected from one or more cellular markers, epitope tags, cytokines, safety switches, dimerization moieties, or degradation moieties.

In another aspect, the present invention provides a chimeric antigen receptor (CAR) encoded by any of the polynucleotides disclosed herein. In some embodiments, the CAR may further comprise one or more additional polypeptide sequences. In some embodiments, the one or more additional polypeptide sequences may be selected from one or more cellular markers, epitope tags, cytokines, safety switches, dimerization moieties, or degradation moieties.

In another aspect, the present invention provides a recombinant vector which may comprise any of the polynucleotides disclosed herein. In some embodiments, the vector may be a viral vector. In some embodiments, the viral vector may be a retroviral vector, a lentiviral vector, an adenoviral vector, an adeno-associated virus vector, an alphaviral vector, a herpes virus vector, a baculoviral vector, or a vaccinia virus vector. In some embodiments, the viral vector may be a lentiviral vector.

In some embodiments, the vector may be a non-viral vector. In some embodiments, the non-viral vector may be a minicircle plasmid, a Sleeping Beauty transposon, a piggyBac transposon, or a single or double stranded DNA molecule that is used as a template for homology directed repair (HDR) based gene editing.

In another aspect, the present invention provides an isolated host cell which may comprise any of the polynucleotides disclosed herein or any of the recombinant vectors disclosed herein.

In another aspect, the present invention provides an isolated host cell which may comprise a chimeric antigen receptor (CAR) encoded by any of the polynucleotides of the present disclosure.

In some embodiments, the host cell may be an immune cell. In some embodiments, the host cell may be a nature killer (NK) cell, T cell, or macrophage. In some embodiments, the host cell may be a natural killer (NK) cell derived from peripheral, cord blood, IPSCs, and/or a cell line (e.g., NK-92 cells). In some embodiments, the host cell may be a T cell. In some embodiments, the host cell may be a CD8+ T-cell, a CD4+ T-cell, a cytotoxic T-cell, an αβ T-cell receptor (TCR) T-cell, an invariant natural killer T (INKT) cell, a γδ T-cell, a memory T-cell, a memory stem T-cell (TSCM), a naïve T-cell, an effector T-cell, a T-helper cell, or a regulatory T-cell (Treg). In some embodiments, the isolated host cell disclosed herein may be an immune cell which is derived from an induced pluripotent stem (IPS) cell.

In some embodiments, any of the isolated host cells disclosed herein may be further genetically modified to enhance its function by expressing one or more additional genes (e.g., transcription factors (e.g. c-Jun) or cytokines (e.g. IL-15); or deleting one or more inhibitory genes (e.g. cytokine inducible SH2 containing protein (CISH), DNA (cytosine-5)-methyltransferase 3A (DNMT3A)) with a gene editing technology (e.g., CRISPR-Cas9, base editors, or transcription activator-like effector nucleases (TALENs)).

In some embodiments, the host cell may be activated and/or expanded ex vivo.

In some embodiments, the host cell may be an allogeneic cell.

In some embodiments, the host cell may be an autologous cell.

In some embodiments, the host cell may be derived from a blood, marrow, tissue, or a tumor sample.

In another aspect, the present invention provides a pharmaceutical composition which may comprise an isolated host cell disclosed herein and a pharmaceutically acceptable carrier and/or excipient.

In another aspect, the present invention provides a method of generating an isolated host cell disclosed herein, said method comprising genetically modifying the host cell with any of the polynucleotides of the present disclosure or of the recombinant vectors of the present disclosure. In some embodiments, the genetic modifying step may be conducted via viral gene delivery. In some embodiments, the genetic modifying step may be conducted via non-viral gene delivery. In some embodiments, the genetic modification may be conducted ex vivo.

In some embodiments, the method disclosed herein may further comprise activation and/or expansion of the host cell disclosed herein ex vivo before, after and/or during said genetic modification.

In another aspect, the present invention provides a method for treating a disease in a subject in need thereof, which may comprise administering to the subject a therapeutically effective amount of any of the host cell(s) of the present disclosure or of the pharmaceutical composition of the present disclosure. In some embodiments, the disease may be a cancer, autoimmune disease, or infectious disease. In some embodiments, treatment methods disclosed herein may comprise: a) isolating NK cells, T cells, or macrophages or from the subject; b) genetically modifying said NK cells, T cells, or macrophages ex vivo with any of the polynucleotides disclosed herein or the vectors disclosed herein; c) optionally, expanding and/or activating said NK cells, T cells, or macrophages before, after or during step (b); and d) introducing the genetically modified NK cells, T cells, or macrophages into the subject. In some embodiments, the subject may be human.

### BRIEF DESCRIPTION OF THE DRAWINGS

FIGS. 1A-1P illustrate that the PDZ binding moiety scaffolding anchor enhances CAR NK cell synapse formation. Chimeric antigen receptor design schemes comprising an antigen recognition domain (anti-ephrin type-A receptor 2 [EphA2] single chain variable fragments [scFv]), a short hinge domain IgG (SH), a transmembrane domain cluster of differentiation (CD) 28 (CD28TM), a CD28 co-stimulatory domain, a CD3ζ activation domain, a Postsynaptic density-95, Discs large, and Zona occludens 1 binding moiety (PDZbm) scaffolding anchor domain, and/or mutated PDZbm domain (FIG. 1A). An exemplary mechanism of action scheme for an enhanced synapse CAR (CAR.PDZ) is also shown (FIG. 1B). Quantification of synaptic area determined by actin immunolabeling, n=17 and 19, UN (untransduced); n=19 and 16, CAR Δ; n=76 and 44, CAR; n=12 and 83, CAR.PDZ with and without EphA2 respectively. Two-Way ANOVA mean±SEM shown of one donor (FIG. 1C). Phospho-Zap-70 (pZAP70) intensity quantification from FIG. 1I-1P. Two-Way ANOVA mean±SEM (standard error of the mean) shown. Cells are the same as in FIG. 1C (FIG. 1D). Lysosome-associated membrane glycoprotein 1 (Lamp1) intensity quantification from FIG. 1I-1P. Two-Way ANOVA mean±SEM shown. Cells are the same as in FIG. 1C (FIG. 1E). Confocal images as prepared in FIG. 1I-1P incubated for 60 minutes with NK cells in various groups quantified in FIGS. 1G-1H. Bars indicate 10 microns. Immunolabeling of Scribble, CD3ε, and filamentous actin (F-actin) (FIG. 1F). Scribble polarization and accumulation at the immune synapse (IS). CAR Δ; n=32, CAR; n=23, CAR.PDZmut: n=57, CAR.PDZ n=28 from two independent experiments, One-Way ANOVA mean±SEM shown of two donors (FIG. 1G). CD3ε co-localization with Scribble as determined by Pearson Correlation Coefficient. CAR Δ; n=33, CAR; n=48, CAR.PDZmut: n=29, CAR.PDZ n=61, One-Way ANOVA mean±SEM shown of one donor (FIG. 1H). Fluorescent confocal microscopy of NK cells with and without recombinant human EphA2 protein incubated for 30 minutes, bars indicate 10 microns; pZAP70, Lamp1, Actin. Dashed line in merged images delineate the approximate line scan quantization of the fluorescent signal depicted in the histograms to the right. Quantified single cell fluorescent data is plotted in FIG. 1C-1E (FIG. 1I-1P). Two-stage linear step-up procedure of Benjamini, Krieger and Yekutieli to correct for false discovery rate (FDR) in all cases.

FIGS. 2A-2H demonstrate that CAR.PDZ NK cells exhibit enhanced avidity and calcium flux upon cancer cell recognition. Single cell assessment of at least NK cell avidity to EphA2-positive A549 tumor cells; at least 100 cells for each independent experiment was analyzed. UN; n=3 CAR Δ; n=4, CAR; n=3, CAR.PDZ; n=4 donors, mean±SEM shown (FIG. 2A). Normalized fold change of CAR NK cell binding compared to untransduced NK cells from FIG. 2A. Arrowed line indicates the point of statistical difference at CAR.PDZ vs. CAR 4 and vs CAR at 548pN which continued to 1000pN. Two-Way ANOVA was used to determine statistical significance with Two-stage linear step-up procedure of Benjamini, Krieger and Yekutieli to correct for FDR; mean±SEM shown (FIG. 2B). Avidity score of CAR NK cells determined by plateau of One-Phase Decay analysis from FIG. 2A. One-Way ANOVA was used to determine statistical significance with Two-stage linear step-up procedure of Benjamini, Krieger and Yekutieli to correct for FDR. mean±SEM shown (FIG. 2C). Same experimental conditions as FIG. 2A except utilizing EphA2 deleted A549 cells (FIG. 2D). Same normalized fold change as described in FIG. 2C utilizing EphA2 deleted A549 cells (FIG. 2E). Representative single cell calcium flux analysis of NK cells interacting with A549 tumor cells over indicated time points. Tumor cells, NK cells, and calcium flux data are visualized. Bar indicates 10 microns (FIG. 2F). Fold Change Calcium flux quantification of NK cells with A549 tumor cells. UN; n=28, CAR Δ; n=35, CAR; n=44, CAR.PDZ; n=48. Two-Way ANOVA was used to determine statistical significance with Two-stage linear step-up procedure of Benjamini, Krieger and Yekutieli to correct for FDR q<0.001 *** at minute 1; 1st peak AUC analysis with unpaired Brown-Forsythe and Welch's ANOVA. mean±SEM shown of one donor (FIG. 2G). Fold Change Calcium flux quantification of NK cells with DIPG007 tumor cells. CAR Δ; n=30, CAR; n=19, CAR.PDZmut: n=26, CAR.PDZ n=43. Two-Way ANOVA was used to determine statistical significance with Two-stage linear step-up procedure of Benjamini, Krieger and Yekutieli to correct for FDR q<0.001 *** at minute 6; 1st peak AUC analysis with unpaired Brown-Forsythe and Welch's ANOVA. mean±SEM shown of one donor (FIG. 2H).

FIGS. 3A-3N show CAR.PDZ NK cells have enhanced and distinct cytokine production. Schematic overview of experimental conditions for secretomics analysis. Single cell secretomic analysis using an IsoLight machine depicting the polyfunctionality of NK cells after exposure to A549 target cells for 4-hours (FIG. 3A). Secretion frequency of selected cytokines from the 32 analyte IsoLight Chip. For each cytokine, bars in the graph representing UN, CAR Δ, CAR and CAR PDZ groups are shown from left to right. Two-Way ANOVA was used to determine statistical significance with Two-stage linear step-up procedure of Benjamini, Krieger and Yekutieli to correct for FDR. n=5 donors, mean±SEM shown (FIG. 3B). All CAR constructs produced significantly more cytokines on the 2-analyte level compared to UN. Two-Way ANOVA was used to determine statistical significance with Two-stage linear step-up procedure of Benjamini, Krieger and Yekutieli to correct for FDR. Statistical difference delineated by q<0.0001 ****. n=5 donors, mean±SEM shown (FIG. 3C). Polyfunctional Strength Index (PSI) of CAR-NK cells detailing cytokine categories of NK cells from FIG. 3B individual cytokines driving PSI variance as indicated in FIG. 3C. Two-Way ANOVA was used to determine statistical significance with Two-stage linear step-up procedure of Benjamini, Krieger and Yekutieli to correct for FDR. n=5 donors, mean±SEM shown. For each protein, bars in the graph representing UN, CAR Δ, CAR and CAR PDZ groups are shown from left to right. (FIG. 3D). Only the effector cytokine group was significantly different between CAR.PDZ and CAR or CAR Δ NK cells. Two-Way ANOVA was used to determine statistical significance with Two-stage linear step-up procedure of Benjamini, Krieger and Yekutieli to correct for FDR. n=5 donors, mean±SEM shown (FIG. 3E). tSNE plots of the secretomic analysis performed in FIGS. 2A-E. tSNE plots revealed distinct cytokine secretion profiles and patterns for each construct tested. These plots highlight the increased secretion frequency and quantity. Log transformed secretion value intensities are delineated. Dashed ellipses indicate groups of highest secretion. Grayscale spectra vary per cytokine from 0 to the highest value in each group, n=5 donors (FIGS. 3F-3N).

FIGS. 4A-4I show CAR.PDZ NK cells have enhanced cytolytic activity and invasive properties. Cytotoxicity assay scheme with A549 lung adenocarcinoma cell viability determined by a chromogenic MTS assay after 24-hour co-culture with NK cells (FIG. 4A). CAR vs CAR.PDZ NK cells: significant differences at all effector-to-target (E:T) ratios except for 5:1. All other comparisons are significantly different. Two-Way ANOVA was used to determine statistical significance with Two-stage linear step-up procedure of Benjamini, Krieger and Yekutieli to correct for FDR. Statistical difference delineated by q<0.05 *, q<0.01 **, n=3 donors, mean±SEM shown (FIG. 4B). CAR vs CAR.PDZ vs CAR.PDZmut NK cells: significant differences at all effector-to-target (E:T) ratios 2.5:1. Two-Way ANOVA was used to determine statistical significance with Two-stage linear step-up procedure of Benjamini, Krieger and Yekutieli to correct for FDR. n=3 donors, mean±SEM shown (FIG. 4C). UN, CAR Δ, CAR, CAR.PDZ NK cells with 10 micromolar PDZ blocking or control (CTRL) peptides. Technical triplicates with mean±SEM shown (FIG. 4D). Area between the curve analysis (shaded regions in FIG. 2D) for two independent experiments with unique donors normalized to untransduced (FIG. 4E). Representative images from a tumoroid droplet cytotoxicity assay 143b osteosarcoma cells in mCherry. Bars indicate 1 mm (FIG. 4F). Quantification of the resulting tumor reduction as determined by tumor area normalized to the 1-hour (hr) mark. CAR.PDZ vs CAR Δ NK cells: significant differences starting at 40 hrs of culture; CAR.PDZ vs. CAR NK cells: significant differences starting at 58 hrs of culture. Two-Way ANOVA was used to determine statistical significance with Two-stage linear step-up procedure of Benjamini, Krieger and Yekutieli to correct for FDR. Dashed lines indicate comparison groups and what hour they become statistically different at least q<0.05*. n=3 donors, mean±SEM shown (FIG. 4G). Representative images from a tumoroid droplet invasion assay showing NK cells. Co-cultures were imaged hourly for 2 days. Bars indicate 1 mm (FIG. 4H). NK cell invasion ratio represents the counted NK cells per mm2 and normalized to the 1-hour mark (FIG. 4I).

FIGS. 5A-5J demonstrate CAR.PDZ NK cells extend survival and eradicate solid tumors in vivo. A549 model timeline (FIG. 5A). 2×10⁶ A549 tumor cells were mixed in pure Matrigel and injected subcutaneously (s.c.) into the dorsal flank of male and female NSG mice. 14 days later mice were treated with a single 10×10⁶ intravenous (i.v.) injection of NK cells. Tumor Alone: n=10, UN: n=10, CAR Δ: n=15, CAR: n=15, CAR.PDZ: n=10 mice data merged from two-three independent experiments with unique donors (FIG. 5B). Kaplan-Meier curves of mice from FIG. 5A median survival rates for each group (days): Tumor Alone: 42, UN: 48.5, CAR Δ: 69, CAR: 64, CAR.PDZ: 80. Log-rank test was used to determine significance. Statistical difference delineated by p<0.05 * (FIG. 5C). LM7 locoregional model timeline (FIG. 5D). 1×10⁶ LM7.ffluc tumor cells were injected intraperitoneally (i.p.) into NSG mice. 7 days later mice were treated with a single 10×10⁶ i.p. dose of NK cells. Tumors were measured by bioluminescence imaging. Tumor Alone: n=10, UN: n=10, CAR Δ: n=10, CAR: n=10, CAR.PDZ: n=5 mice merged from two independent experiments with unique donors (FIG. 5E). Kaplan-Meier curves of mice from FIG. 5B median survival rates for each group (days): Tumor Alone: 47, UN: 56, CAR Δ: 66.5, CAR: 76, CAR.PDZ: undefined. Log-rank test was used to determine significance. Statistical difference delineated by p<0.05 *, <0.01 ** (FIG. 5F). 143b model timeline (FIG. 5G). 1×10⁶ 143b tumor cells were injected subcutaneously (s.c.) into the dorsal flank of NSG mice. 5 days later mice were treated with a single 10×10⁶ intravenous (i.v.) injection of NK cells. Tumor Alone: n=10, UN: n=10, EphA2 CARs: CAR Δ: n=5, CAR: n=5, CAR.PDZ: n=4. B7 Homolog 3 (B7-H3) CARs CAR Δ: n=4, CAR: n=5, CAR.PDZ: n=5 mice (FIG. 5H). Kaplan-Meier curves of EphA2 targeted CAR NK treated mice from FIG. 5H median survival rates (days): Tumor Alone: 28, UN: 28, CAR Δ: 30, CAR: 28, CAR.PDZ: 42. Log-rank test was used to determine significance. Statistical difference delineated by p<0.05 *, <0.01 ** (FIG. 5I). Kaplan-Meier curves of B7-H3 targeted CAR NK treated mice from FIG. 5H median survival rates (days): Tumor Alone: 28, UN: 28, CAR Δ: 26, CAR: 28, CAR.PDZ: 42. Log-rank test was used to determine significance. Statistical difference delineated by p<0.01 ** (FIG. 5J).

FIGS. 6A-6G demonstrate CAR.PDZ T cells extend survival and eradicate solid tumors in vivo. Example images of B7-H3 CAR and CAR.PDZ T cells, tumor cells, synapse, calcium flux (FIG. 6A). Immune synapse area quantification of B7-H3 CAR and CAR.PDZ T cells (n=10 and 16 cells) with Area Under the Curve (AUC) analysis with mean±SEM shown of one donor (FIG. 6B). Calcium flux quantification of B7-H3 CAR and CAR.PDZ T cells (n=14 and 28 cells) with Two-Way ANOVA was used to determine statistical significance with Two-stage linear step-up procedure of Benjamini, Krieger and Yekutieli to correct for FDR q<0.001 *** at minute 2; 1st peak AUC analysis with unpaired Student's t-Test with Welch's correction. mean±SEM shown of one donor (FIG. 6C). DIPG007 model timeline (FIG. 6D). 1×10⁶ DIPG007 tumor cells were injected intracranially (i.e.) into NSG mice. 7 days later mice were treated with 2×10⁶ intracranial (i.e.) injection of T cells. Tumor Alone: n=7, CAR Δ: n=10, CAR: n=9, CAR.PDZ: n=9 mice from two independent pooled experiments mean±SEM shown (FIG. 6E). Net AUC analysis of tumor burden with One-Way ANOVA was used to determine statistical significance with Two-stage linear step-up procedure of Benjamini, Krieger and Yekutieli to correct for FDR. mean±SEM shown (FIG. 6F). DIPG7c model timeline (FIG. 6G). 1.5×10⁵ DIPG7c tumor cells were injected intracranially (i.e.) into the striatum of male and female NSG mice. 7 days later mice were treated with 2×10⁶ intracranial (i.e.) injection of T cells. Each group of mice is n=5, one donor (FIG. 6H). Kaplan-Meier curves of mice from FIG. 1G median survival rates (days): Tumor Alone: 27, CAR Δ: 27, CAR: 48, CAR.PDZ: Undefined. Log-rank test was used to determine significance. Statistical difference delineated by p<0.01 ** (FIG. 6I). 143b model timeline (FIG. 6J). 1×10⁶ 143b tumor cells were injected subcutaneously (s.c.) into the dorsal flank of NSG mice. 7 days later mice were treated with 10×10⁶ intravenous (i.v.) injection of T cells. Tumor Alone: n=4, CAR: n=10, CAR.PDZ: n=9 mice merged from two unique donors. Two-stage linear step-up procedure of Benjamini, Krieger and Yekutieli to correct for FDR. Statistical difference delineated by q<0.0001 ****, mean±SEM shown (FIG. 6K). Kaplan-Meier curves of B7-H3 targeted CAR T treated mice from FIG. 6K median survival rates (days): Tumor Alone: 29, CAR Δ: 29, CAR: 28, CAR.PDZ: 41. Log-rank test was used to determine significance. Statistical difference delineated by p<0.001 *** (FIG. 6L).

FIGS. 7A-7C illustrate primary NK cell transduction efficiency. Representative flow cytometry histogram plots detailing surface CAR expression (FIG. 7A). Quantified flow cytometry data showing percent CAR positive NK cells of various donors. UN: n=9, CAR Δ: n=8, CAR: n=9, CAR.PDZ: n=6 donors, mean±SEM shown (FIG. 7B). Immunophenotype of NK cells via flow cytometry. n=4 donors, mean±SEM shown (FIG. 7C).

FIGS. 8A-8B show Scribble Polarization at 30 minutes. Confocal images as prepared in FIG. 1 incubated for 30 minutes with NK cells in various groups quantified in FIG. 8B. Bars indicate 10 microns. Immunolabeling of Scribble, CD3ε, and filamentous actin (F-actin) (FIG. 8A). Scribble polarization and accumulation at the immune synapse (IS). CAR Δ; n=13, CAR; n=7, CAR.PDZmut: n=14, CAR.PDZ n=9, One-Way ANOVA was used to determine statistical significance with Two-stage linear step-up procedure of Benjamini, Krieger, and Yekutieli to correct for FDR. mean±SEM shown of one donor (FIG. 8B).

FIG. 9 illustrates Wiskott/Aldrich syndrome protein (WASp) Polarization at 15 and 30 minutes. Confocal images as prepared in FIG. 1 incubated for 15 and 30 minutes with NK cells in various groups. Quantified WASp polarization and accumulation at the immune synapse (IS). CAR 4; n=34 and 25, CAR; n=42 and 25, CAR.PDZ n=19 and 39, for 15 and 30 minutes, respectively. Two-Way ANOVA was used to determine statistical significance with Two-stage linear step-up procedure of Benjamini, Krieger, and Yekutieli to correct for FDR. Statistical difference delineated by q<0.01 *, q<0.0001 ****; mean±SEM shown of one donor

FIG. 10 shows that live NK cell imaging reveals lysosomal condensing and enhanced synapse formation with increased calcium flux. Lysosomal coalescing from live cell imaging in FIG. 2G EphA2 targeting CARs UN; n=27, CAR Δ; n=48, CAR; n=44, CAR.PDZ; n=48 cells. Peak lysosome signal was measured from the first calcium flux peak in each condition. One-Way ANOVA was used to determine statistical significance with Two-stage linear step-up procedure of Benjamini, Krieger and Yekutieli to correct for FDR; mean±SEM shown of one donor

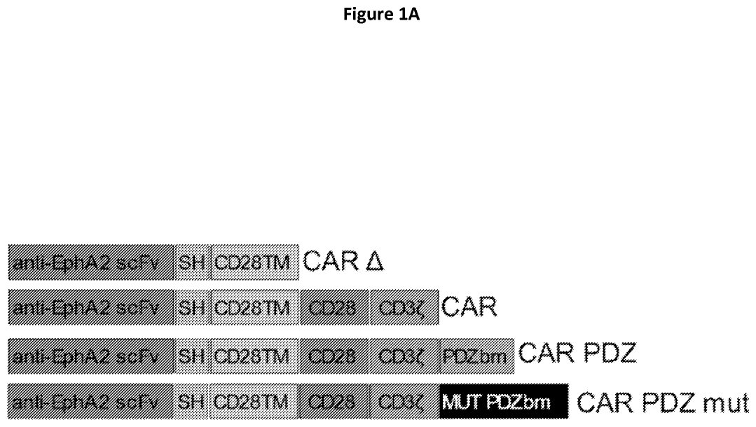

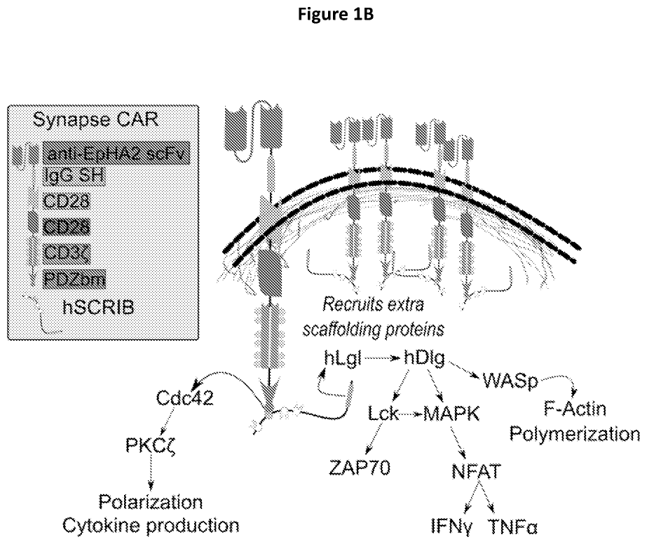

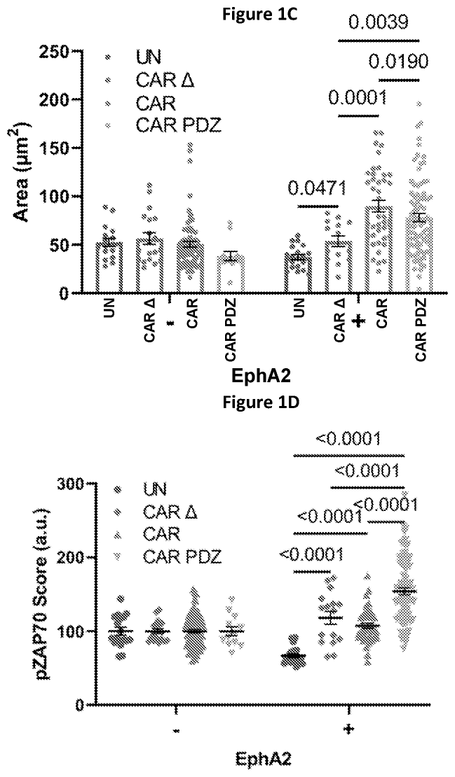

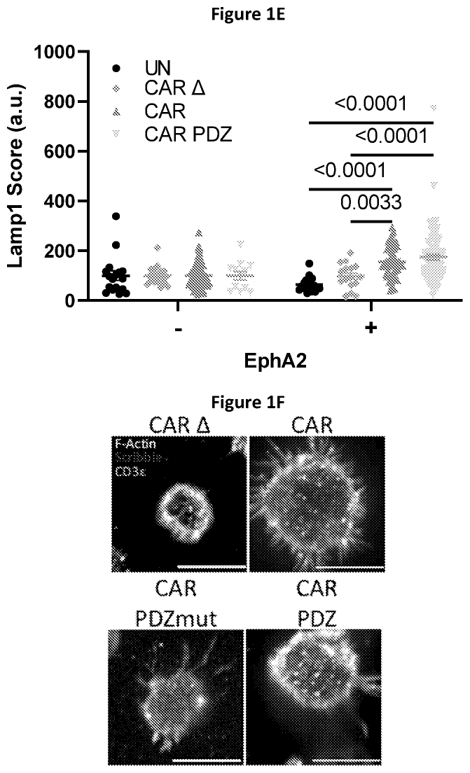

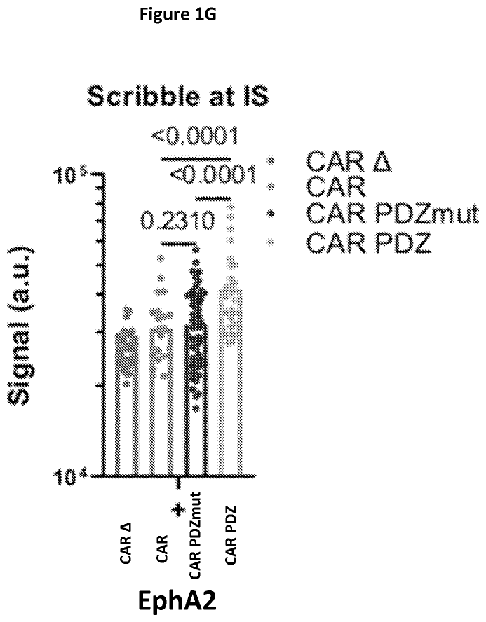

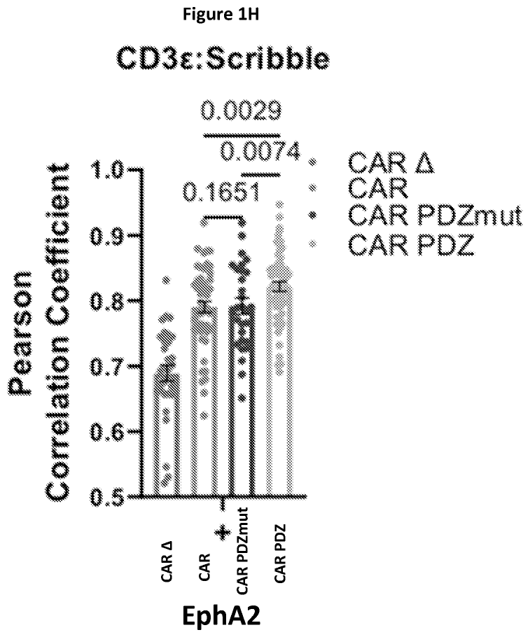

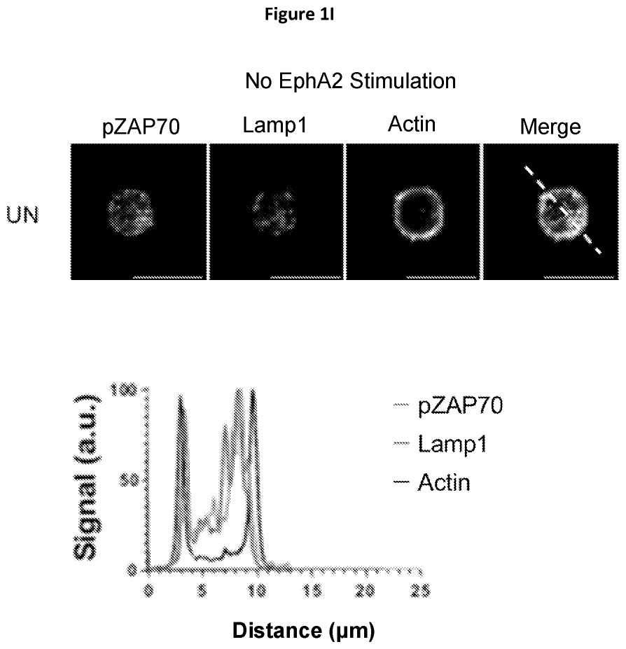

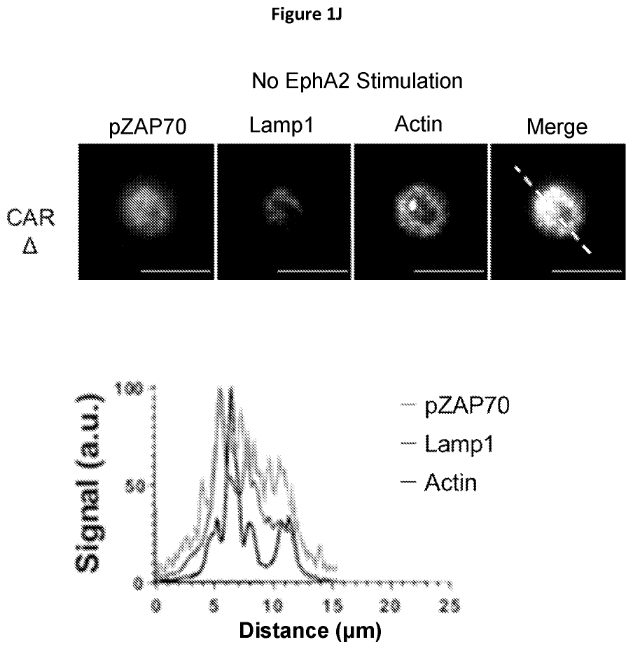

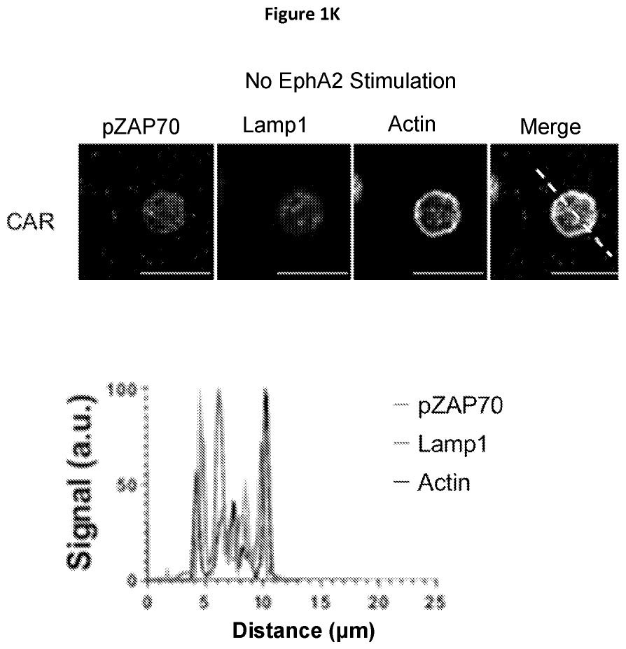

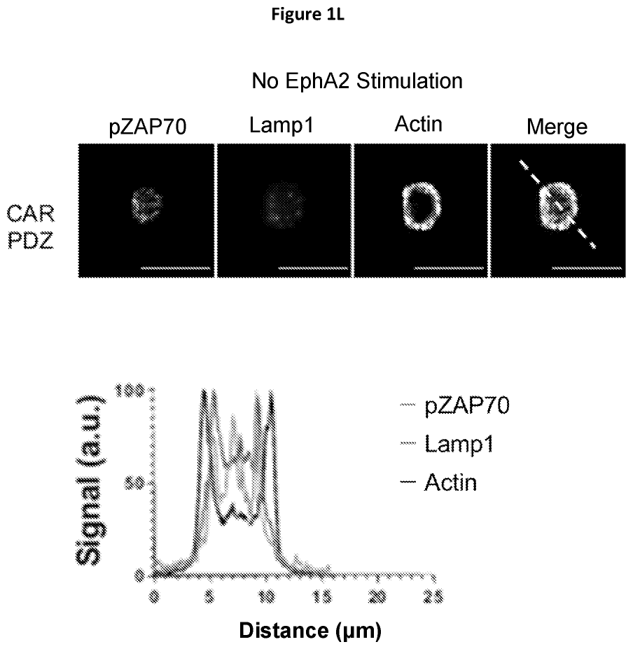

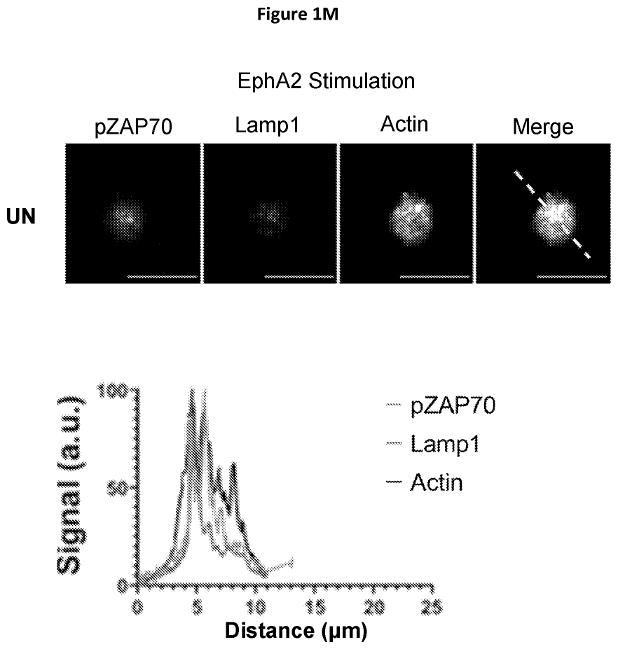

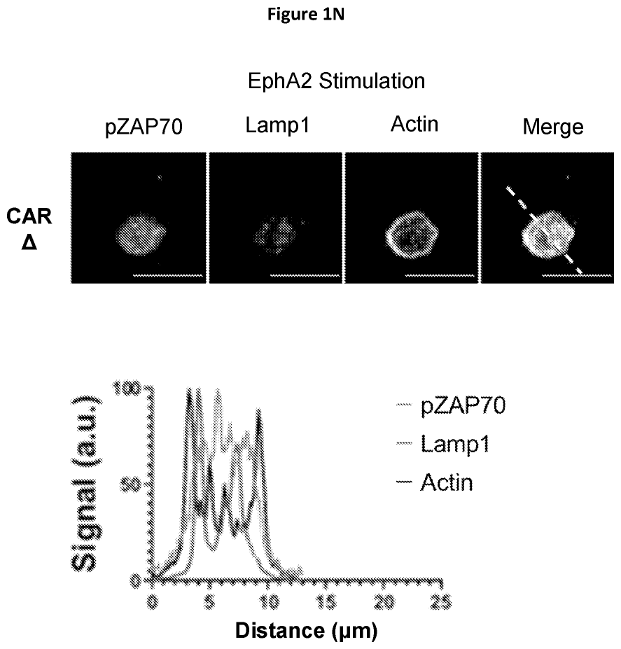

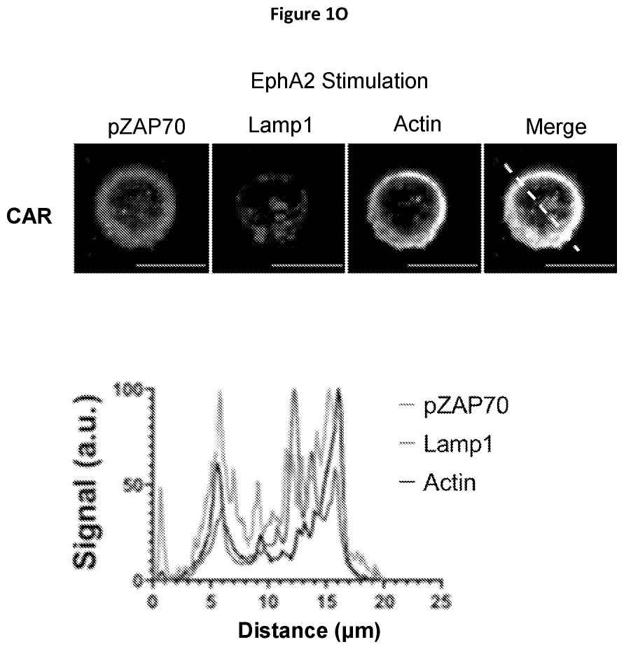

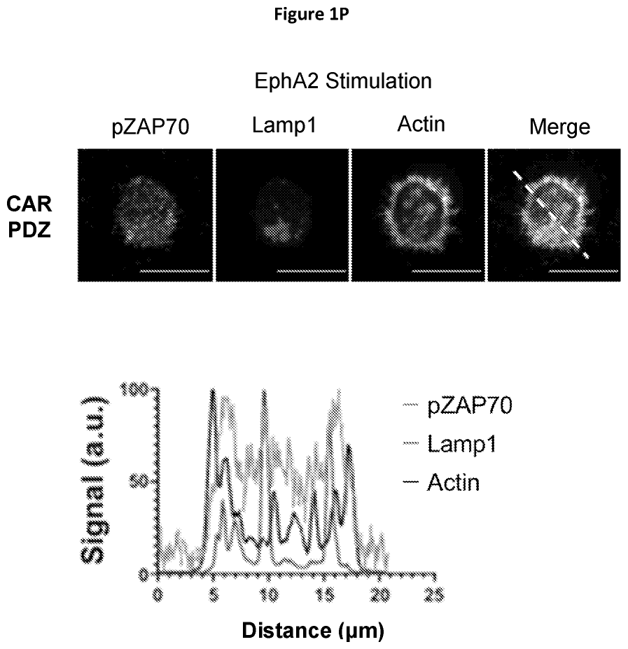

FIG. 1I shows LM7 Avidity assessment with EphA2 targeting CARs. Normalized fold change of CAR NK cell binding compared to untransduced NK cells. Arrowed lines indicate the point of statistical difference at CAR.PDZ vs. CAR Δ at 268pN, CAR.PDZ vs CAR at 343pN, CAR.PDZ vs CAR.PDZmut at 363pN which continued to 1000pN indicated by dashed arrow lines. The only exception to this significance was from 650 to 738pN for CAR.PDZ vs CAR.PDZmut. Two-Way ANOVA was used to determine statistical significance with Two-stage linear step-up procedure of Benjamini, Krieger and Yekutieli to correct for FDR; n=1-3 donors, mean±SEM shown

FIGS. 12A-12E depict B7-H3 CAR design with avidity, synapse, and calcium flux analyses. Chimeric antigen receptor design schemes showing antigen recognition domain (anti-B7-H3 scFv), hinge and transmembrane domains (CD8αH/TM), CD28 co-stimulatory domain, CD3ζ activation domain, and/or PDZbm scaffolding anchor domain (FIG. 12A). Example flow cytometry plot detailing B7-H3 CAR expression (FIG. 12B). Normalized fold change of CAR NK cell binding compared to untransduced NK cells. Bracketed line indicates the scale of statistical difference at CAR.PDZ vs. CAR Δ and CAR from 194 to 646pN for both comparisons except for 205-215pN. Two-Way ANOVA was used to determine statistical significance with Two-stage linear step-up procedure of Benjamini, Krieger and Yekutieli to correct for FDR; q<0.05 *, <0.001 ***, n=3 donors, mean±SEM shown (FIG. 12C). Immune synapse area quantification of B7-H3 CAR and CAR.PDZ NK cells (n=11 and 11 cells) Two-Way ANOVA was used to determine statistical significance with Uncorrected Fisher's LSD test p<0.05 * at minute 5 and 12 with area under the curve analysis (FIG. 12D). Calcium flux quantification of B7-H3 CAR and CAR.PDZ NK cells (n=15 and 14 cells) with Two-Way ANOVA was used to determine statistical significance with Uncorrected Fisher's LSD test p<0.05 * starting at minute 2; 1st peak AUC analysis with unpaired Student's t-Test, mean±SEM shown of one donor. (FIG. 12E)

FIGS. 13A-13C illustrate A549 and LM7 tumor rechallenge rejection. A549 tumor rechallenge timeline with identical initial cancer cell numbers. Indicated tumor volumes from palpable nodules overtime (FIG. 13A). Intravital imaging of LM7 rechallenge with identical initial cancer cell numbers in complete responder mice. Grayscale 1e6 to 1e7 of total photon flux (p/s) (FIG. 13B). Tumor flux values of weekly measurements (FIG. 13C).

FIGS. 14A-14F depict CAR T cell phenotyping and cytokine production. Chimeric antigen receptor design schemes showing antigen recognition domain (anti-B7-H3 scFv), hinge and transmembrane domains (CD8αH/TM), CD28 co-stimulatory domain, CD3ζ activation domain, and/or PDZbm scaffolding anchor domain (FIG. 14A). Transduction efficiencies of various CAR constructs in T cells CAR Δ; n=7, CAR; n=8, CAR.PDZ n=5 donors mean±SEM shown (FIG. 14B). CD4/8 T cell analysis post transduction at Day 5-6, n=4 donors mean±SEM shown (FIG. 14C). Immunophenotype of CD4 CAR T cells Day 5-6 and longitudinally Day 12-13, n=4 donors mean±SEM shown (FIG. 14D). Immunophenotype of CD8 CAR T cells Day 5-6 and longitudinally Day 12-13, n=4 donors mean±SEM shown (FIG. 14E). B7-H3 CAR T cells were co-cultured with A549, LM7, and 143b cancer cells lines at a 2:1 ratio for 24 hours for n=3 experiments. Supernatant was collected and multiplex cytokine assessment was performed. One-Way ANOVA was used to determine statistical significance with Two-stage linear step-up procedure of Benjamini, Krieger and Yekutieli to correct for FDR mean±SEM (FIG. 14F).

FIGS. 15A-15D show B7-H3 CAR T cell synapse and calcium flux analyses. Immune synapse area quantification of B7-H3 CAR and CAR.PDZ T cells (n=12 and 9 cells) co-cultured with LM7 cells with AUC analysis (FIG. 15A). Calcium flux quantification of B7-H3 CAR and CAR.PDZ T cell (n=16 and 22 cells) co-cultured with LM7 cells with Two-Way ANOVA was used to determine statistical significance with Uncorrected Fisher's LSD test p<0.0001 **** at minute 4; 1st peak AUC analysis. mean±SEM shown of one donor (FIG. 15B). Calcium flux quantification of B7-H3 CAR and CAR.PDZ T cells (n=42 and 15 cells) co-cultured with U87 cells. Two-Way ANOVA was used to determine statistical significance with Uncorrected Fisher's LSD test p<0.0001 *** starting at minute 1; 1st peak AUC analysis with unpaired Student's t-Test. mean±SEM shown of one donor (FIG. 15C). Calcium flux quantification of B7-H3 CAR and CAR.PDZ T cells (n=22 and 31 cells) co-cultured with DIPG007 cells. Two-Way ANOVA was used to determine statistical significance with Uncorrected Fisher's LSD test p<0.0001 **** starting at minute 1; 1st peak AUC analysis with unpaired Student's t-Test. mean±SEM shown of one donor (FIG. 15D).

FIG. 16 shows B7-H3 expression on tumor cells. 143B, U87, LM7, DIPG7c, and DIPG007 tumor cells were analyzed for B7-H3 expression. Histograms indicate isotype controls and immunolabeled cells. Each sample is 100% positive and the indicated gMFI values are delineated.

FIG. 17 shows pERK quantification of stimulated CAR NK cells. CAR-NK cells were stimulated with rhEphA2 for indicated time points. BD PhosFlow kit was performed to manufacturer's instruction. Percent pERK positive CAR-NK cells are indicated, n=1.

FIGS. 18A-18C show nucleotide sequences and amino acid sequences for exemplary CARs of the present disclosure. Nucleotide sequence and amino acid sequences for an exemplary non-signaling CAR, comprising EphA2 scFv, short hinge, and CD28 transmembrane domains (FIG. 18A). Nucleotide sequence and amino acid sequences for an exemplary standard CAR, comprising EphA2 scFv, short hinge, CD28 transmembrane, CD28 costimulatory, and CD3ζ (CD3 zeta) activation domains (FIG. 18B). Nucleotide sequence and amino acid sequences for an exemplary PDZ CAR, comprising EphA2 scFv, short hinge, CD28 transmembrane, CD28 costimulatory, CD3ζ (CD3 zeta) domains, and PDZ binding motif (PFZbm) (FIG. 18C).

FIGS. 19A-19B show amino acid sequences (FIG. 19A) and nucleotide sequences (FIG. 19B) for an exemplary B7-H3 CAR PDZ construct described herein.

FIGS. 20A-20B show amino acid sequences (FIG. 20A) and nucleotide sequences (FIG. 20B) for an exemplary B7-H3 CAR construct described herein.

FIGS. 21A-21B show amino acid sequences (FIG. 21A) and nucleotide sequences (FIG. 21B) for an exemplary non-signaling B7-H3 CAR construct described herein.

### DETAILED DESCRIPTION

In various aspects, the present disclosure relates to methods for tuning CAR synapses in immune cells, for example, by adding an intracellular scaffolding protein binding site to the CAR, and compositions comprising the synapse-tuned CAR-modified cells. A PDZ binding motif (PDZbm), in particular, was employed that specifically binds Scribble⁴ resulting in additional scaffolding crosslinkings that enhance synapse formation and NK CAR cell polarization^{5,6}. Combined effects of this CAR design resulted in increased effector cell functionality in vitro and in vivo. T cells were utilized and similar global enhancements of in effector function were observed. By way of a non-limiting example, synapse-tuned CAR-NK cells exhibited amplified synaptic strength, number and abundance of secreted cytokines, enhanced killing of tumor cells, and prolonged survival with tumor clearance in two solid tumor models. Thus, synapse tuning may improve the efficacy of CAR-based cell therapeutics.

### Definitions

The term “chimeric antigen receptor” or “CAR” as used herein is defined as a cell-surface receptor comprising an extracellular target-binding domain, a transmembrane domain, and a cytoplasmic domain comprising a lymphocyte activation domain and optionally at least one co-stimulatory signaling domain, all in a combination that is not naturally found together on a single protein. This particularly includes receptors wherein the extracellular domain and the cytoplasmic domain are not naturally found together on a single receptor protein. The chimeric antigen receptors of the present disclosure can be used with lymphocyte such as T-cells and natural killer (NK) cells.

The term “cell polarity protein” refers to any of various proteins capable of regulating or modifying spatial differences in shape, structure, and/or function within a cell, e.g., a eukaryotic cell, including an immune cell. By way of a non-limiting example, such proteins may participate in synapse formation, migration, organization, and/or replication.

The terms “T cell” and “T lymphocyte” are interchangeable and used synonymously herein. As used herein, T-cell includes thymocytes, naive T lymphocytes, immature T lymphocytes, mature T lymphocytes, resting T lymphocytes, or activated T lymphocytes. A T-cell can be a T helper (Th) cell, for example a T helper 1 (Th1) or a T helper 2 (Th2) cell. The T-cell can be a helper T-cell (HTL; CD4+ T-cell) CD4+ T-cell, a cytotoxic T-cell (CTL; CD8+ T-cell), a tumor infiltrating cytotoxic T-cell (TIL; CD8+ T-cell), CD4+CD8+ T-cell, or any other subset of T-cells. Other illustrative populations of T-cells suitable for use in particular embodiments include naive T-cells and memory T-cells. Also included are “NKT cells”, which refer to a specialized population of T-cells that express a semi-invariant αβ T-cell receptor, but also express a variety of molecular markers that are typically associated with NK cells, such as NK1.1. NKT cells include NK1.1+ and NK1.1−, as well as CD4+, CD4−, CD8+ and CD8− cells. The TCR on NKT cells is unique in that it recognizes glycolipid antigens presented by the MHC I-like molecule CD Id. NKT cells can have either protective or deleterious effects due to their abilities to produce cytokines that promote either inflammation or immune tolerance. Also included are “gamma-delta T-cells (γδ T-cells),” which refer to a specialized population that to a small subset of T-cells possessing a distinct TCR on their surface, and unlike the majority of T-cells in which the TCR is composed of two glycoprotein chains designated α- and β-TCR chains, the TCR in γδ T-cells is made up of a γ-chain and a δ-chain. γδ T-cells can play a role in immunosurveillance and immunoregulation, and were found to be an important source of IL-17 and to induce robust CD8+ cytotoxic T-cell response. Also included are “regulatory T-cells” or “Tregs” refers to T-cells that suppress an abnormal or excessive immune response and play a role in immune tolerance. Tregs cells are typically transcription factor Foxp3-positive CD4+ T cells and can also include transcription factor Foxp3-negative regulatory T-cells that are IL-10-producing CD4+ T cells.

The terms “natural killer cell” and “NK cell” are used interchangeable and used synonymously herein. As used herein, NK cell refers to a differentiated lymphocyte with a CD 16+CD56+ and/or CD57+ TCR− phenotype. NKs are characterized by their ability to bind to and kill cells that fail to express “self” MHC/HLA antigens by the activation of specific cytolytic enzymes, the ability to kill tumor cells or other diseased cells that express a ligand for NK activating receptors, and the ability to release protein molecules called cytokines that stimulate or inhibit the immune response.

As used herein, the term “antigen” refers to any agent (e.g., protein, peptide, polysaccharide, glycoprotein, glycolipid, nucleic acid, portions thereof, or combinations thereof) molecule capable of being bound by a T-cell receptor. An antigen is also able to provoke an immune response. An example of an immune response may involve, without limitation, antibody production, or the activation of specific immunologically competent cells, or both. A skilled artisan will understand that an antigen need not be encoded by a “gene” at all. It is readily apparent that an antigen can be generated synthesized or can be derived from a biological sample or might be macromolecule besides a polypeptide. Such a biological sample can include, but is not limited to a tissue sample, a tumor sample, a cell or a fluid with other biological components, organisms, subunits of proteins/antigens, killed or inactivated whole cells or lysates.

The term “antigen-binding moiety” refers to a target-specific binding element that may be any ligand that binds to the antigen of interest or a polypeptide or fragment thereof, wherein the ligand is either naturally derived or synthetic. Examples of antigen-binding moieties include, but are not limited to, antibodies; polypeptides derived from antibodies, such as, for example, single chain variable fragments (scFv), Fab, Fab′, F(ab′)2, and Fv fragments; polypeptides derived from T-cell receptors, such as, for example, TCR variable domains; secreted factors (e.g., cytokines, growth factors) that can be artificially fused to signaling domains (e.g., “zytokines”); and any ligand or receptor fragment (e.g., CD27, NKG2D) that binds to the antigen of interest. Combinatorial libraries could also be used to identify peptides binding with high affinity to the therapeutic target.

Terms “antibody” and “antibodies” refer to monoclonal antibodies, multispecific antibodies, human antibodies, humanized antibodies, chimeric antibodies, single-chain Fvs (scFv), single chain antibodies, Fab fragments, F(ab′) fragments, disulfide-linked Fvs (sdFv), intrabodies, minibodies, diabodies and anti-idiotypic (anti-Id) antibodies (including, e.g., anti-Id antibodies to antigen specific TCR), and epitope-binding fragments of any of the above. The terms “antibody” and “antibodies” also refer to covalent diabodies such as those disclosed in U.S. Pat. Appl. Pub. 2007/0004909 and Ig-DARTS such as those disclosed in U.S. Pat. Appl. Pub. 2009/0060910. Antibodies useful as a TCR-binding molecule include immunoglobulin molecules and immunologically active fragments of immunoglobulin molecules, i.e., molecules that contain an antigen-binding site. Immunoglobulin molecules can be of any type (e.g., IgG, IgE, IgM, IgD, IgA and IgY), class (e.g., IgG1, IgG2, IgG3, IgG4, IgM1, IgM2, IgA1 and IgA2) or subclass.

The term “host cell” means any cell that contains a heterologous nucleic acid. The heterologous nucleic acid can be a vector (e.g., an expression vector). For example, a host cell can be a cell from any organism that is selected, modified, transformed, grown, used or manipulated in any way, for the production of a substance by the cell, for example the expression by the cell of a gene, a DNA or RNA sequence, a protein or an enzyme. An appropriate host may be determined. For example, the host cell may be selected based on the vector backbone and the desired result. By way of example, a plasmid or cosmid can be introduced into a prokaryote host cell for replication of several types of vectors. Bacterial cells such as, but not limited to DH5α, JM109, and KCB, SURE® Competent Cells, and SOLOPACK Gold Cells, can be used as host cells for vector replication and/or expression. Additionally, bacterial cells such as E. coli LE392 could be used as host cells for phage viruses. Eukaryotic cells that can be used as host cells include, but are not limited to yeast (e.g., YPH499, YPH500 and YPH501), insects and mammals. Examples of mammalian eukaryotic host cells for replication and/or expression of a vector include, but are not limited to, HeLa, NIH3T3, Jurkat, 293, COS, CHO, Saos, and PC12. In certain embodiments, the host cell is autologous. In certain embodiments, the host cell is allogenic.

Host cells of the present disclosure include T-cells and natural killer cells that contain the DNA or RNA sequences encoding the CAR and express the CAR on the cell surface. Such host cells may be used for enhancing T-cell activity, natural killer cell activity, treatment of tumors, and treatment of autoimmune disease.

The terms “activation” or “stimulation” means to induce a change in their biologic state by which the cells (e.g., T-cells and NK cells) express activation markers, produce cytokines, proliferate and/or become cytotoxic to target cells. All these changes can be produced by primary stimulatory signals. Co-stimulatory signals can amplify the magnitude of the primary signals and suppress cell death following initial stimulation resulting in a more durable activation state and thus a higher cytotoxic capacity. A “co-stimulatory signal” refers to a signal, which in combination with a primary signal, such as TCR/CD3 ligation, leads to T-cell and/or NK cell proliferation and/or upregulation or downregulation of key molecules.

The term “proliferation” refers to an increase in cell division, either symmetric or asymmetric division of cells. The term “expansion” refers to the outcome of cell division and cell death.

The term “differentiation” refers to a method of decreasing the potency or proliferation of a cell or moving the cell to a more developmentally restricted state.

The terms “express” and “expression” mean allowing or causing the information in a gene or DNA sequence to become produced, for example producing a protein by activating the cellular functions involved in transcription and translation of a corresponding gene or DNA sequence. A DNA sequence is expressed in or by a cell to form an “expression product” such as a protein. The expression product itself, e.g., the resulting protein, may also be said to be “expressed” by the cell. An expression product can be characterized as intracellular, extracellular or transmembrane.

The term “transfection” means the introduction of a “foreign” (i.e., extrinsic or extracellular) nucleic acid into a cell using recombinant DNA technology. The term “genetic modification” means the introduction of a “foreign” (i.e., extrinsic or extracellular) gene, DNA or RNA sequence to a host cell, so that the host cell will express the introduced gene or sequence to produce a desired substance, typically a protein or enzyme coded by the introduced gene or sequence. The introduced gene or sequence may also be called a “cloned” or “foreign” gene or sequence, may include regulatory or control sequences operably linked to polynucleotide encoding the chimeric antigen receptor, such as start, stop, promoter, signal, secretion, or other sequences used by a cell's genetic machinery. The gene or sequence may include nonfunctional sequences or sequences with no known function. A host cell that receives and expresses introduced DNA or RNA has been “genetically engineered.” The DNA or RNA introduced to a host cell can come from any source, including cells of the same genus or species as the host cell, or from a different genus or species.

The term “transduction” means the introduction of a foreign nucleic acid into a cell using a viral vector.

The terms “genetically modified” or “genetically engineered” refers to the addition of extra genetic material in the form of DNA or RNA into a cell.

As used herein, the term “derivative” or “variant” in the context of proteins or polypeptides (e.g., CAR constructs or domains thereof) refer to: (a) a polypeptide that has at least 40%, 45%, 50%, 55%, 60%, 65%, 70%, 75%, 80%, 85%, 90%, 95%, 98% or 99% sequence identity to the polypeptide it is a derivative or variant of; (b) a polypeptide encoded by a nucleotide sequence that has at least 40%, 45%, 50%, 55%, 60%, 65%, 70%, 75%, 80%, 85%, 90%, 95%, 98% or 99% sequence identity to a nucleotide sequence encoding the polypeptide it is a derivative or variant of; (c) a polypeptide that contains 1, 2, 3, 4, 5, 6, 7, 8, 9, 10, 11, 12, 13, 14, 15, 16, 17, 18, 19, 20 or more amino acid mutations (i.e., additions, deletions and/or substitutions) relative to the polypeptide it is a derivative or variant of; (d) a polypeptide encoded by nucleic acids can hybridize under high, moderate or typical stringency hybridization conditions to nucleic acids encoding the polypeptide it is a derivative or variant of; (e) a polypeptide encoded by a nucleotide sequence that can hybridize under high, moderate or typical stringency hybridization conditions to a nucleotide sequence encoding a fragment of the polypeptide, it is a derivative or variant of, of at least 20 contiguous amino acids, at least 30 contiguous amino acids, at least 40 contiguous amino acids, at least 50 contiguous amino acids, at least 75 contiguous amino acids, at least 100 contiguous amino acids, at least 125 contiguous amino acids, or at least 150 contiguous amino acids; or (f) a fragment of the polypeptide it is a derivative or variant of.

Percent sequence identity can be determined using any method known to one of skill in the art. In a specific embodiment, the percent identity is determined using the “Best Fit” or “Gap” program of the Sequence Analysis Software Package (Version 10; Genetics Computer Group, Inc., University of Wisconsin Biotechnology Center, Madison, Wisconsin). Information regarding hybridization conditions (e.g., high, moderate, and typical stringency conditions) have been described, see, e.g., U.S. Patent Application Publication No. US 2005/0048549 (e.g., paragraphs 72-73).

The terms “vector”, “cloning vector” and “expression vector” mean the vehicle by which a DNA or RNA sequence (e.g., a foreign gene) can be introduced into a host cell, so as to genetically modify the host and promote expression (e.g., transcription and translation) of the introduced sequence. Vectors include plasmids, synthesized RNA and DNA molecules, phages, viruses, etc. In certain embodiments, the vector is a viral vector such as, but not limited to, viral vector is an adenoviral, adeno-associated, alphaviral, herpes, lentiviral, retroviral, or vaccinia vector.

The term “regulatory element” refers to any cis-acting genetic element that controls some aspect of the expression of nucleic acid sequences. In some embodiments, the term “promoter” comprises essentially the minimal sequences required to initiate transcription. In some embodiments, the term “promoter” includes the sequences to start transcription, and in addition, also include sequences that can upregulate or downregulate transcription, commonly termed “enhancer elements” and “repressor elements”, respectively.

As used herein, the term “operatively linked” and similar phrases, when used in reference to nucleic acids or amino acids, refer to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA). In some embodiments, operatively linked nucleic acid elements result in the transcription of an open reading frame and ultimately the production of a polypeptide (i.e., expression of the open reading frame). As another example, an operatively linked peptide is one in which the functional domains are placed with appropriate distance from each other to impart the intended function of each domain.

By “enhance” or “promote” or “increase” or “expand” or “improve” refers generally to the ability of a composition contemplated herein to produce, elicit, or cause a greater physiological response (i.e., downstream effects) compared to the response caused by either vehicle or a control molecule/composition. A measurable physiological response may include an increase in T-cell expansion, activation, effector function, persistence, and/or an increase in tumor cell death killing ability, among others apparent from the understanding in the art and the description herein. In certain embodiments, an “increased” or “enhanced” amount can be a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7, 1.8, etc.) the response produced by vehicle or a control composition.

By “decrease” or “lower” or “lessen” or “reduce” or “abate” refers generally to the ability of composition contemplated herein to produce, elicit, or cause a lesser physiological response (i.e., downstream effects) compared to the response caused by either vehicle or a control molecule/composition. In certain embodiments, a “decrease” or “reduced” amount can be a “statistically significant” amount, and may include a decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7, 1.8, etc.) the response (reference response) produced by vehicle, a control composition, or the response in a particular cell lineage.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition, but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

The term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The phrase “pharmaceutically acceptable”, as used in connection with compositions described herein, refers to molecular entities and other ingredients of such compositions that are physiologically tolerable and do not typically produce untoward reactions when administered to a mammal (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

The term “protein” is used herein encompasses all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation, pegylation, biotinylation, etc.).

The terms “nucleic acid”, “nucleotide”, and “polynucleotide” encompass both DNA and RNA unless specified otherwise. By a “nucleic acid sequence” or “nucleotide sequence” is meant the nucleic acid sequence encoding an amino acid, the term may also refer to the nucleic acid sequence including the portion coding for any amino acids added as an artifact of cloning, including any amino acids coded for by linkers.

The terms “patient”, “individual”, “subject”, and “animal” are used interchangeably herein and refer to mammals, including, without limitation, human and veterinary animals (e.g., cats, dogs, cows, horses, sheep, pigs, etc.) and experimental animal models. In a preferred embodiment, the subject is a human.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

Singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

If aspects of the disclosure are described as “comprising” a feature, or versions there of (e.g., comprise), embodiments also are contemplated “consisting of” or “consisting essentially of” the feature.

The practice of the present disclosure employs, unless otherwise indicated, conventional techniques of statistical analysis, molecular biology (including recombinant techniques), microbiology, cell biology, and biochemistry, which are within the skill of the art. Such tools and techniques are described in detail in e.g., Sambrook et al. (2001) Molecular Cloning: A Laboratory Manual. 3rd ed. Cold Spring Harbor Laboratory Press: Cold Spring Harbor, New York; Ausubel et al. eds. (2005) Current Protocols in Molecular Biology. John Wiley and Sons, Inc.: Hoboken, NJ; Bonifacino et al. eds. (2005) Current Protocols in Cell Biology. John Wiley and Sons, Inc.: Hoboken, NJ; Coligan et al. eds. (2005) Current Protocols in Immunology, John Wiley and Sons, Inc.: Hoboken, NJ; Coico et al. eds. (2005) Current Protocols in Microbiology, John Wiley and Sons, Inc.: Hoboken, NJ; Coligan et al. eds. (2005) Current Protocols in Protein Science, John Wiley and Sons, Inc.: Hoboken, NJ; and Enna et al. eds. (2005) Current Protocols in Pharmacology, John Wiley and Sons, Inc.: Hoboken, NJ. Additional techniques are explained, e.g., in U.S. Pat. No. 7,912,698 and U.S. Patent Appl. Pub. Nos. 2011/0202322 and 2011/0307437.

The technology illustratively described herein suitably may be practiced in the absence of any element(s) not specifically disclosed herein.

The terms and expressions which have been employed are used as terms of description and not of limitation, and use of such terms and expressions do not exclude any equivalents of the features shown and described or portions thereof, and various modifications are possible within the scope of the technology claimed.

### Chimeric Antigen Receptors

In certain aspects, the present disclosure provides a polynucleotide encoding a CAR comprising: (a) an extracellular domain, (b) a transmembrane domain, and (c) a cytoplasmic domain comprising a signaling domain and an anchoring domain which binds to a cell polarity protein.

In some embodiments, the extracellular domain comprises an antigen-binding moiety, wherein the antigen-binding moiety may comprise, for example, an antibody or an antibody fragment. In some embodiments, the antigen-binding moiety may comprise a single chain variable fragment (scFv) such as, but not limited to, an EphA2 scFv or a B7-H3 scFv (see, e.g., FIGS. 18A-18C or, e.g., FIGS. 19A-19B, FIGS. 20A-20B, and FIGS. 21A-21B). In some embodiments, the antigen-binding moiety may comprise a ligand or peptide sequence. In some embodiments, the antigen-binding moiety may comprise a heavy chain variable region (VH) sequence, a light chain variable region (VL) sequence, and/or CDRs disclosed herein. In some embodiments, the antigen-binding moiety may comprise an scFv derived from an antibody or antibody fragment that binds to an antigen target disclosed herein. In some embodiments, the antigen-binding moiety may comprise an antigen-binding moiety derived from a CAR that binds to an antigen target. In some embodiments, the antigen-binding moiety may bind to a tumor antigen, antigen of extracellular matrix, antigen present on cells within the tumor microenvironment, tissue-specific antigen, autoimmune antigen or infectious antigen disclosed herein.

In some embodiments, the cell polarity protein may comprise a Postsynaptic density-95, Discs large, and Zona occludens 1 (PDZ) domain.

In some embodiments, the anchoring domain of any of various CARs of the present disclosure may comprise a PDZ binding motif (PDZbm). In some embodiments, when the CAR of the disclosure comprises a PDZbm, the CAR comprising said PDZbm may be referred to herein as a “PDZ CAR” or “CAR PDZ” or “CAR.PDZ”.

### Extracellular Domain

In certain aspects, CARs of the present disclosure comprise an extracellular domain, wherein the extracellular domain comprises an antigen-binding moiety.

The choice of antigen-binding moiety depends upon the type and number of antigens that define the surface of a target cell. For example, the antigen-binding moiety may be chosen to recognize an antigen that acts as a cell surface marker on target cells associated with a particular disease state. In certain embodiments, the CARs of the present disclosure can be genetically modified to target a tumor antigen of interest by way of engineering a desired antigen-binding moiety that specifically binds to an antigen (e.g., on a tumor cell). Non-limiting examples of cell surface markers that may act as targets for the antigen-binding moiety in the CAR of the disclosure include those associated with tumor cells, extracellular matrix, extracellular matrix, cells within the tumor microenvironment, specific tissues, autoimmune disease, and/or infectious diseases.

In some embodiments, the antigen-binding moiety may bind to a tumor antigen. In some embodiments, the antigen-binding moiety may bind to an antigen of extracellular matrix. In some embodiments, the antigen-binding moiety may bind to an antigen present on cells within the tumor microenvironment. In some embodiments, the antigen-binding moiety may bind to an antigen that is tissue specific. In some embodiments, the antigen-binding moiety may bind to an antigen that is tissue non-specific. In some embodiments, the antigen-binding moiety may bind to infectious antigen.

In certain embodiments, the antigen-binding moiety can be monomeric or multimeric (e.g., homodimeric or heterodimeric), or associated with multiple proteins in a non-covalent complex. In some embodiments, the antigen-binding moiety comprises an antigen-binding peptide, polypeptide or functional variant thereof that binds to an antigen.

In some embodiments, the antigen-binding moiety is an antibody or an antibody fragment that binds to an antigen. Antigen-binding moieties may comprise antibodies and/or antibody fragments such as monoclonal antibodies, multispecific antibodies, chimeric antibodies, single-chain Fvs (scFv), single chain antibodies, Fab fragments, F(ab′) fragments, disulfide-linked Fvs (sdFv), intrabodies, minibodies, single domain antibody variable domains, nanobodies (VHHs), diabodies and anti-idiotypic (anti-Id) antibodies (including, e.g., anti-Id antibodies to antigen specific TCR), and epitope-binding fragments of any of the above. Antibodies and/or antibody fragments may be derived from murine antibodies, rabbit antibodies, human antibodies, fully humanized antibodies, camelid antibody variable domains and humanized versions, shark antibody variable domains and humanized versions, and camelized antibody variable domains.

In some embodiments, the antigen-binding moiety may be a ligand. Non-limiting examples of CARs comprising an antigen-binding moiety that is a ligand include IL-13 mutein-CARs or CD27-CARs. In some embodiments, the antigen-binding moiety may be a peptide sequence. Non-limiting examples of CARs comprising an antigen-binding moiety that is a peptide sequence include chlorotoxin and GRP78-CARs. See, for example, PCT Patent Application WO/2021/216994, which is herein incorporated by reference in its entirety.

In some embodiments, the antigen-binding moiety binds to at least one tumor antigen. In some embodiments, the antigen-binding moiety binds to two or more tumor antigens. In some embodiments, the two or more tumor antigens are associated with the same tumor. In some embodiments, the two or more tumor antigens are associated with different tumors.

In some embodiments, the antigen-binding moiety binds to at least one antigen of extracellular matrix. In some embodiments, the antigen-binding moiety binds to two or more antigens of the extracellular matrix. In some embodiments, the two or more tumor antigens are associated with the same extracellular matrix. In some embodiments, the two or more tumor antigens are associated with different extracellular matrix.

In some embodiments, the antigen-binding moiety binds to at least one antigen present on cells within the tumor microenvironment. In some embodiments, the antigen-binding moiety binds to two or more antigens present on cells within the tumor microenvironment. In some embodiments, the two or more antigens are associated with the same cell. In some embodiments, the two or more tumor antigens are associated with different cells.

In some embodiments, the antigen-binding moiety binds to at least one autoimmune antigen. In some embodiments, the antigen-binding moiety binds to two or more autoimmune antigens. In some embodiments, the two or more autoimmune antigens are associated with the same autoimmune disease. In some embodiments, the two or more autoimmune antigens are associated with different autoimmune diseases.

In some embodiments, the antigen-binding moiety binds to at least one infectious antigen. In some embodiments, the antigen-binding moiety binds to two or more infectious antigens. In some embodiments, the two or more infectious antigens are associated with the same infectious disease. In some embodiments, the two or more infectious antigens are associated with different infectious diseases.

In some embodiments, the tumor antigen is associated with glioblastoma, ovarian cancer, cervical cancer, head and neck cancer, liver cancer, prostate cancer, pancreatic cancer, renal cell carcinoma, bladder cancer, or hematologic malignancy. Non-limiting examples of the tumor antigens associated with cervical cancer or head and neck cancer include MUC1, Mesothelin, HER2, GD2, and EGFR. Non-limiting examples of tumor antigens associated with ovarian cancer include FOLR1, FSHR, MUC16, MUC1, Mesothelin, CA125, EpCAM, EGFR, PDGFRα, Nectin-4, B7-H3 and B7-H4. Non-limiting examples of tumor antigens associated with hematological malignancies include BCMA, GPRC5D, SLAM F7, CD33, CD19, CD22, CD79, CLL1, CD123, and CD70. Non-limiting examples of tumor antigens associated with bladder cancer include Nectin-4 and SLITRK6. Non-limiting examples of tumor antigens associated with renal cancer include CD70 and FOLR1. Non-limiting examples of tumor antigen associated with glioblastoma include FGFR1, FGFR3, MET, CD70, ROBO1, IL13Rα2, HER2, EGFRVIII, EGFR, CD133, and PDGFRA. Non-limiting examples of tumor antigen associated with liver cancer include, EpCAM, cMET, AFP, Claudin 18.2, and GPC-3.

Additional examples of antigens that may be targeted by the antigen-binding moiety include, but are not limited to, alpha-fetoprotein, A3, antigen specific for A33 antibody, Ba 733, BrE3-antigen, carbonic anhydrase Ep-CAM, EphA1, EphA2, B7-H3, EphA3, EphA4, EphA5, EphA6, EphA7, EphA8, EphA10, EphB1, EphB2, EphB3, EphB4, EphB6, FIt-I, Flt-3, folate receptor, HLA-DR, human chorionic gonadotropin (HCG) and its subunits, hypoxia inducible factor (HIF-I), Ia, IL-2, IL-6, IL-8, insulin growth factor-1 (IGF-I), KC4-antigen, KS-1-antigen, KS1-4, Le-Y, macrophage inhibition factor (MIF), MAGE, MUC2, MUC3, MUC4, NCA66, NCA95, NCA90, EX, EGFR, EGP-I, EGP-2, antigen specific for PAM-4 antibody, placental growth factor, p53, prostatic acid phosphatase, PSA, PSMA, RS5, S100, CD1, CD1a, CD3, CD5, CD15, CD16, CD19, CD20, CD21, CD22, CD23, CD25, CD30, CD33, CD38, CD45, CD74, CD79a, CD80, CD123, CD138, colon-specific antigen-p (CSAp), CEA (CEACAM5), CEACAM6, CSAp, TAC, TAG-72, tenascin, VEGF, ED-B fibronectin, COL11A1, 17-1A-antigen, TRAIL receptors, Tn antigen, Thomson-Friedenreich antigens, tumor necrosis antigens, an oncogene marker, an oncogene product, or an angiogenesis marker.

In some embodiments, the antigen is associated with an autoimmune disease or disorder. Such antigens may be derived from cell receptors and cells which produce “self”-directed antibodies. In some embodiments, the antigen is associated with an autoimmune disease or disorder such as, psoriasis, vasculitis, Wegener's granulomatosis, Hashimoto's thyroiditis, Graves' disease, chronic inflammatory demyelinating polyneuropathy, Guillain-Barre syndrome, Crohn's disease, ulcerative colitis, Rheumatoid arthritis (RA), multiple sclerosis (MS), Sjögren's syndrome, Systemic lupus erythematosus, sarcoidosis, Type 1 diabetes mellitus, insulin dependent diabetes mellitus (IDDM), autoimmune thyroiditis, reactive arthritis, ankylosing spondylitis, scleroderma, polymyositis, dermatomyositis, or Myasthenia gravis.

In some embodiments, autoimmune antigens that may be targeted by the CAR disclosed herein include but are not limited to islet cell antigen, platelet antigens, Sm antigens in snRNPs, myelin protein antigen, Rheumatoid factor, and anticitrullinated protein, glucose-6-phosphate isomerase, receptors such as lipocortin 1, neutrophil nuclear proteins such as lactoferrin and 25-35 kD nuclear protein, granular proteins such as bactericidal permeability increasing protein (BPI), elastase fibrinogen, fibrin, vimentin, filaggrin, collagen I and II peptides, alpha-enolase, citrullinated proteins and peptides such as CCP-1, CCP-2 (cyclical citrullinated peptides), translation initiation factor 4G1, perinuclear factor, keratin, Sa (cytoskeletal protein vimentin), circulating serum proteins such as RFs (IgG, IgM), fibrinogen, plasminogen, components of articular cartilage such as collagen II, IX, and XI, ferritin, nuclear components such as RA33/hnRNP A2, Sm, stress proteins such as HSP-65, -70, -90, BiP, inflammatory/immune factors such as B7-H1, IL-1 alpha, and IL-8, enzymes such as calpastatin, alpha-enolase, eukaryotic translation elongation factor 1 alpha 1aldolase-A, dipeptidyl peptidase, osteopontin, cathepsin G, myeloperoxidase, proteinase 3, antigen, islet cell antigen, rheumatoid factor, histones, ribosomal P proteins platelet antigens, myelin protein, cardiolipin, vimentin, nucleic acids such as, and RNA, ribonuclear particles and proteins such as Sm antigens (including but not limited to SmD's and SmB′/B), U1RNP, A2/B1 hnRNP, Ro (SSA), and La (SSB) antigens, dsDNA, and ssDNA.

In some embodiments, the antigen targeted by CARs of the present disclosure is an antigen expressed in the tumor stroma. Exemplary antigens expressed in the tumor stroma that may be targeted by CARs of the present disclosure include but are not limited to oncofetal splice variants of fibronectin and tenascin C, tumor-specific splice variants of collagen, and fibroblast activating protein (FAP).

In some embodiments, the antigen targeted by CARs of the present disclosure is an antigen expressed on endothelial cell. Exemplary antigens expressed on endothelial cells that may be targeted by CARs of the present disclosure include, but are not limited to, VEGF receptors, and tumor endothelial markers (TEMs).

Exemplary infectious associated antigens that may be targeted by the modified host cells of the present disclosure include those derived from Adenoviridae (most adenoviruses); Arena viridae (hemorrhagic fever viruses); Birnaviridae; Bungaviridae (e.g., Hantaan viruses, bunga viruses, phleboviruses and Nairo viruses); Calciviridae (e.g., strains that cause gastroenteritis); Coronoviridae (e.g., coronaviruses); Filoviridae (e.g., ebola viruses); Flaviridae (e.g., dengue viruses, encephalitis viruses, yellow fever viruses); Hepadnaviridae (Hepatitis B virus; HBsAg); Herpesviridae (herpes simplex virus (HSV) 1 and 2, varicella zoster virus, cytomegalovirus (CMV), herpes virus); Iridoviridae (e.g., African swine fever virus); Norwalk and related viruses, and astroviruses; Orthomyxoviridae (e.g., influenza viruses); Papovaviridae (papilloma viruses, polyoma viruses); Paramyxoviridae (e.g., parainfluenza viruses, mumps virus, measles virus, respiratory syncytial virus); Parvovirida (parvoviruses); Picornaviridae (e.g., polio viruses, hepatitis A virus; enteroviruses, human Coxsackie viruses, rhinoviruses, echoviruses); Poxviridae (variola viruses, vaccinia viruses, pox viruses); Reoviridae (e.g., reoviruses, orbiviurses and rotaviruses); Retroviridae (e.g., human immunodeficiency viruses, such as HIV-1 (also referred to as HTLV-III, LAV or HTLV-III/LAV, or HIV-III); and other isolates, such as HIV-LP); Rhabdoviradae (e.g., vesicular stomatitis viruses, rabies viruses); Togaviridae (e.g., equine encephalitis viruses, rubella viruses); and unclassified viruses (e.g., the etiological agents of Spongiform encephalopathies, the agent of delta hepatitis, the agents of non-A, non-B hepatitis (i.e. Hepatitis C)).

Additional infectious antigens that may be targeted by the modified host cells of the present disclosure include bacterial antigens, fungal antigens, parasite antigens, or prion antigens, or the like. Non-limiting examples of infectious bacteria include but are not limited to: Actinomyces israelli, Bacillus antracis, Bacteroides sp., Borelia burgdorferi, Chlamydia., Clostridium perfringers, Clostridium tetani, Corynebacterium diphtheriae, Corynebacterium sp., Enterobacter aerogenes, Enterococcus sp., Erysipelothrix rhusiopathiae, Fusobacterium nucleatum, Haemophilus influenzae, Helicobacter pyloris, Klebsiella pneumoniae, Legionella pneumophilia, Leptospira, Listeria monocytogenes, Mycobacteria sps. (e.g., M tuberculosis, M avium, M gordonae, M intracellulare, M kansaii), Neisseria gonorrhoeae, Neisseria meningitidis, Pasteurella multocida, pathogenic Campylobacter sp., Rickettsia, Staphylococcus aureus, Streptobacillus monihformis, Streptococcus (anaerobic sps.), Streptococcus (viridans group), Streptococcus agalactiae (Group B Streptococcus), Streptococcus bovis, Streptococcus faecalis, Streptococcus pneumoniae, Streptococcus pyogenes (Group A Streptococcus), Treponema pallidium, and Treponema pertenue. Non-limiting examples of infectious fungi include: Cryptococcus neoformans, Histoplasma capsulatuin, Coccidioides immitis, Blastomyces dernatitidis, Chlamydia trachomatis and Candida albicans. Other infectious organisms (i.e., protists) include: Plasmodium such as Plasmodium falciparum, Plasmodium malariae, Plasmodium ovale, Plasmodium vivax, Toxoplasma gondii and Shistosoma. Other medically relevant microorganisms have been descried extensively in the literature, e.g., see C. G. A. Thomas, “Medical Microbiology”, Bailliere Tindall, Great Britain 1983, which is hereby incorporated by reference in its entirety.

Other examples of antigens that may be targeted by the modified host cells of the present disclosure include antigens expressed on immune and/or stem cells to deplete these cells such as CD45RA and c-kit.

Various non-limiting exemplary antigen targets are also displayed in Tables 1-3.

In some embodiments, the antigen-binding moiety may comprise a VH sequence, a VL sequence, and/or CDRs thereof, such as those described in the cited publications, the contents of each publication are incorporated herein by reference in their entirety for all purposes (Table 1).

TABLE 1
Exemplary antigen-binding moities comprising a VH sequence,
a VL sequence, and/or CDRs thereof

| Antigen target | Type | Examples of Source | Type | Examples of Source |
|---|---|---|---|---|
| Oncofetal | — | — | VL | Identifier 1, 2, 7 in |
| fibronectin | | | | US20070202103A1 |
| VMS2 | VH | FIG. 1 in | — | — |
| | | WO2000058363 | | |
| GCC1 | VH | Identifier 1 in | VL | Identifier 4, 2, in |
| | | US20160030595A1 | | US20160030595A1 |
| MVR | VH | Identifier 1 in | VL | Identifier 5 in |
| | | US20160257762A1 | | US20160257762A1 |
| OlfmB | VH | Identifier 1 in | VL | Identifier 2 in |
| | | WO2015054441A1 | | WO2015054441A1 |
| TRBC1 | VH | Identifier 1 in | VL | Identifier 2 in |
| | | WO2015132598 | | WO2015132598 |
| Malignant | VH | Identifier 1 in | VL | Identifier 5 in |
| Variable | | WO2015133817A1 | | WO2015133817A1 |
| Receptor | | | | |
| CD40 | VH | Identifier 1 in | VL | Identifier 2 in |
| | | WO2016069919; | | WO2016069919; |
| | | Identifier 5, 7, 8 in | | Identifier 6 in |
| | | WO2015091655 | | WO2015091655 |
| CDIM | VH | Identifier 1, 10, 11, | VL | Identifier 28, 29, |
| | | 12, 13, 14, 15, 16, | | 30, 31, 32, 33, 34, |
| | | 17, 18, 19, 2, 20, | | 35, 36, 37, 38, 39, |
| | | 21, 22, 3, 4, 5, 6, 7, | | 40, 41, 42, 43, 44, |
| | | 8, 9 in | | 45, 46, 47, 48, 49, |
| | | WO2013120012 | | in WO2013120012 |
| ILIRAP | VH | Identifier 1, 10, 19, | VL | Identifier 14, 15, 17, |
| | | 8, 9 in | | 18, 2, 20 in |
| | | WO2016020502; | | WO2016020502; |
| | | Identifier 120, 122, | | Identifier 121, 123, |
| | | 124 in | | 125 in |
| | | WO2016179319A1 | | WO2016179319A1 |
| ALK | VH | Identifier 1, 11, 13, | VL | Identifier 10, 12, 14, |
| | | 15, 3, 5, 7, 9 in | | 16, 2, 4, 6, 8 in |
| | | US20160280798A1; | | US20160280798A1; |
| | | Identifier 9, 1, 3, | | Identifier 10, 12, 14, |
| | | 5, 11, 13, 15, 7, 9 in | | 16, 8, in |
| | | WO2015069922 | | WO2015069922 |
| LHR | VH | Identifier 1, 2, 3, 4, | — | — |
| | | 5, 6, 7, 8 in | | |
| | | WO2016160618A3 | | |
| MUC16 | VH | Identifier 1, 21, 41, | VL | Identifier 2, 22, 42, |
| | | 81, 61 in | | 62, 82 in |
| | | WO2016149368; | | WO2016149368 |
| | | Identifier 11, 4, 6, | | |
| | | 8 in | | |
| | | US20130171152 | | |
| PTK7 | VH | Identifier 1, 25, 49 | VL | Identifier 20, 22, 24, |
| | | in US20150315293; | | 26, 28, 30, 32, 34, |
| | | Identifier 21, 23, 25, | | 36, 38, 40, 42, 44, |
| | | 27, 29, 31, 33, 35, | | 46, 48, 50, 52, 54, |
| | | 37, 39, 41, 43, 45, | | 56, 58, 60, 62, 64, |
| | | 47, 49, 51, 53, 55, | | 66, 68 in |
| | | 57, 59, 61, 63, 65, | | WO2012112943A1 |
| | | 67, 69 in | | Identifier 15, 39, 63 |
| | | WO2012112943A1 | | in US20150315293 |
| ANG2 | VH | Identifier 1, 3 in | VL | Identifier 2, 4 in |
| | | WO2015091655 | | WO2015091655 |
| CD71 | VH | Identifier 1, 3, 325, | VL | Identifier 2, 327, |
| | | 4, 5, 699, in | | 329, 331, 333, 335, |
| | | US20160355599 | | 337, 6, 650, 652, |
| | | | | 654, 656, 658, 660, |
| | | | | 670, 671, 672, 673, |
| | | | | 7, 701, 702, 703, |
| | | | | 704, 705, 706, 707, |
| | | | | 708, 709, 710, 711, |
| | | | | 712, 721, 722, 723, |
| | | | | 724, 725, 726, 727, |
| | | | | 728, 729, 730, 731, |
| | | | | 732, 733, 734, 735, |
| | | | | 736, 737, 738, 739, |
| | | | | 740, 741, 742, 743, |
| | | | | 744, 745, 746, 747, |
| | | | | 748, 749, 750, 751, |
| | | | | 752, 753, 754, 755, |
| | | | | 756, 757, 758, 759, |
| | | | | 760, 761, 762, 763, |
| | | | | 764, 765, 766, 767, |
| | | | | 768, 769, 770, 771, |
| | | | | 772, 773, 774, 775, |
| | | | | 776, 777, 778, 779, |
| | | | | 780, 781, 782, 783, |
| | | | | 784 785, 786, 787, |
| | | | | 788, 8, 810, 811, |
| | | | | 812, 813, 814, 815, |
| | | | | 816, 817, 818, 819, |
| | | | | 820, 821, 822, 823, |
| | | | | 824, 825, 826, 827, |
| | | | | 828, 829, 830, 831, |
| | | | | 832, 833, 834, 835, |
| | | | | 836, 810, 842, 843, |
| | | | | 844, 845, 846, 847, |
| | | | | 848, 849, 850, 851, |
| | | | | 852, 853, 854, 855, |
| | | | | 856, 857, 858, 859, |
| | | | | 860, 861, 862, 863, |
| | | | | 864, 865, 866, 867, |
| | | | | 868, 869, 870, 871, |
| | | | | 872, 873, 874, 875, |
| | | | | 876, 877, 879, 879, |
| | | | | 880, 881, 882, 883, |
| | | | | 884, 885, 886, 887, |
| | | | | 888, 889, 890, 891, |
| | | | | 892, 893, 894, 895, |
| | | | | 896, 897, 898, 899, |
| | | | | 900, 901, 902, 903, |
| | | | | 905, 906, 907, 908, |
| | | | | in US20160355599 |
| TEM8 | VH | Identifier 1, 3, 5, 7 in | VL | Identifier 4, 6, 8 in |
| | | US20160264662A1 | | US20160264662A1 |
| IGFI | VH | Identifier 1, 3, 7 in | VL | Identifier 2, 4, 6, 8 in |
| | | WO2007118214; | | WO2007118214; |
| | | Identifier 7 in | | |
| | | WO2015073575 | | |
| FAP | VH | Identifier 1, 5 in | VL | Identifier 2, 6 in |
| | | WO2015118030; | | WO2015118030; |
| | | Identifier 170, 172 in | | Identifier 171, 173 in |
| | | WO2016120216; | | WO2016120216; |
| | | Identifier 8 in | | Identifier 9 in |
| | | US20160326265 A1 | | US20160326265 A1 |
| Mesothelin | VH | Identifier 1, 6 in | VL | Identifier 3, 5 in |
| | | WO2015188141; | | WO2015188141; |
| | | Identifier 119, 50 in | | Identifier 1, 2, 3 in |
| | | US20160333114A1; | | WO2013142034; |
| | | Identifier 5, 6 in | | Identifier 11, 15, 19, |
| | | WO2013142034; | | 23, 27 in |
| | | Identifier 15, 2 in | | US20160229919A1 |
| | | US9416190B2; | | US9416190B2; |
| | | Identifier 13, 17, 21, | | Identifier 120, 47, 49 |
| | | 25, 29, 9 in | | in |
| | | US20160229919A1 | | US20160333114A1 |
| Frizzled | VH | Identifier 10 in | VL | Identifier 12, 14 in |
| Receptor | | WO2010037041 | | WO2010037041 |
| APCDD1 | VH | Identifier 10, 102, | VL | Identifier 136, 100, |
| | | 106, 110, 114, 118, | | 104, 108, 112, 116, |
| | | 122, 126, 130, 134, | | 12, 120, 124, 128, |
| | | 14, 6, 98 in | | 132, 16, 8 in |
| | | WO2012019061 | | WO2012019061 |
| CSF | VH | Identifier 10, 102, 14, | VL | Identifier 12, 32, 44, |
| | | 18, 2, 22, 26, 30, 34, | | 48, 60 in |
| | | 38, 46, 50, 54, 58, 6, | | US20050059113A1 |
| | | 62, 66, 70, 74, 78, | | |
| | | 82, 86, 90, 94, 98 in | | |
| | | US20050059113A1 | | |
| TIGIT | VH | Identifier 10, 11, 12, | VL | Identifieer 130, 131, |
| | | 124, 125, 126, 127, | | 132, 133, 137, 139, |
| | | 128, 129, 13, 136, | | 145, 146, 151, 152, |
| | | 138, 14, 143, 144, | | 25, 26, 27, 28, 29, |
| | | 149, 15, 150, 16, 17, | | 30, 50, 51, 52, 64, |
| | | 18, 19, 20, 21, 22, 23, | | 95, 8 in |
| | | 24, 37, 38, 39, 40, | | US20160355589 |
| | | 41, 42, 43, 44, 45, | | |
| | | 46, 47, 63, 94, 7, 9 | | |
| | | in US20160355589 | | |
| B7H3 | VH | Identifier 10, 11, 12, | VL | Identifier 1, 2, 3, 4, |
| | | 13, 14, 15, 16, 9 in | | 5, 6, 7, 8 in |
| | | WO2016033225 | | WO2016033225 |
| IL4R | VH | Identifier 10, 11, 14, | VL | Identifier 13, 7, 8 in |
| | | 15, 9 in | | WO2009121847 |
| | | WO2009121847 | | |
| Lymphotoxin | VH | Identifier 10, 12, 14, | VL | Identifier 1, 15, 4, |
| beta receptor | | 16, 2 in | | 6, 8 in |
| | | WO2004002431 | | WO2004002431 |
| LGR5 | VH | Identifier 10, 12, 16, | VL | Identifier 15, 19, 21, |
| | | 18, 20, 22, 24, 26, 4 | | 23, 25, 3 in |
| | | in US20160102146 | | US20160102146 |
| CD148 | VH | Identifier 10, 14, 18, | VL | Identifier 12, 16, 20, |
| | | 2, 22, 26, 30, 6 in | | 24, 28, 32, 4, 8 in |
| | | WO2005118643 | | WO2005118643 |
| GPC3 | VH | Identifier 10, 14, 2, 3, | VL | Identifier 10, 14, 18, |
| | | 4, 5, 6, 7, 8, 9 in | | 22, 24, 26 in |
| | | US20160208015 A1; | | US9409994B2; |
| | | Identifier 22 in | | Identifier 16, 31 in |
| | | WO2016049459; | | US20160208015A1; |
| | | Identifier 12, 16, 20, | | Identifier 23 in |
| | | 37, 8 in | | WO2016049459 |
| | | US9409994B2 | | |
| CSPG4 | VH | Identifier 10, 16, 18, | VL | Identifier 7 in |
| | | 4, 6, 8 in | | WO2016164429; |
| | | WO2016077638; | | Identifier 12, 14 in |
| | | Identifier 8 in | | WO2016077638 |
| | | WO2016164429 | | |
| AGR2 | VH | Identifier 10, 18 in | VL | Identifier 11, 19 in |
| | | WO2016040321 | | WO2016040321 |
| Tissue factor | VH | Identifier 10, 19, 23, | VL | Identifier 25. 31 in |
| | | 27, 29, 6 in | | US20040229301A1; |
| | | WO2004094475; | | Identifier 12, 21, 25, |
| | | Identifier 38 in | | 31, 8 in |
| | | US20160333114A1 | | WO2004094475; |
| | | | | Identifier 35, 37 in |
| | | | | US20160333114A1 |
| SEMA- | VH | Identifier 10, 25, 9 in | VL | Identifier 17, 18, 29 |
| PHORIN 4D | | US20160115240A1 | | in US20160115240A1 |
| PDL1 | VH | Identifier 10, 32, 8 in | VL | Identifier 22, 26, 34, |
| | | US20160319022; | | 42, 58, 66, 74, 82, 86 |
| | | Identifier 18, 30, 38, | | in WO2016061142; |
| | | 46, 50, 54, 62, 70, | | Identifier 30, 8, 9 in |
| | | 78 in | | US20150190506; |
| | | WO2016061142; | | Identifier 7, 9 in |
| | | Identifier 29, 7 in | | US20160319022; |
| | | US20150190506; | | Identifier 17, 22, 24, |
| | | Identifier 16, 18, | | 249, 26, 28, 309, 311, |
| | | 197, 247, 248, 250, | | 313, 320, 325, 34, 340, |
| | | 251, 252, 253, 254, | | 357, 359, 36, 42, 44, |
| | | 255, 256, 257, 258, | | 58, 60, 66, 68, 74, 76, |
| | | 259, 260, 30, 308, | | 8, 82, 84, 86, 88 in |
| | | 310, 312, 319, 32, | | US20160108123 |
| | | 324, 339, 356, 38, | | |
| | | 40, 46, 48, 50, 52, | | |
| | | 54, 6, 62, 70, 72, 78, | | |
| | | 80, 91, 96 in | | |
| | | US20160108123; | | |
| | | Identifier 358, 56, | | |
| | | 64 in | | |
| | | US20160108123 | | |
| HLAG | VH | Identifier 10, 8 in | VL | Identifier 18, 20 in |
| | | WO2016160622A2 | | WO2016160622A2 |
| B7H4 | VH | Identifier 100, 101, | VL | Identifier 104, 11, 126, |
| | | 102, 103, 107, 108, | | 134, 138, 19, 27, 3, |
| | | 109, 110, 111, 112, | | 35, 55, 93, 95, 97, 98, |
| | | 113, 114, 12, 127, | | 145, 146, 147, 148 in |
| | | 130, 131, 132, 133, | | US20160159910; |
| | | 137, 2, 20, 28, 36, | | Identifier 29, 31, 33 in |
| | | 37, 38, 4, 56, 99, | | WO2016160620 |
| | | 144 in | | |
| | | US201606159910; | | |
| | | Identifier 13, 15, 17 | | |
| | | in WO2016160620 | | |
| VEGFR2 | VH | Identifier 100, 101, | VL | Identifier 107, 108, |
| | | 102, 103, 114, 115, | | 109, 110, 111, 112, |
| | | 116, 117, 118, 119, | | 113, 86, 87, 88, 89, |
| | | 120, 121, 122, 123, | | 90, 91, 92, 93, 94 in |
| | | 124, 95, 96, 97, 98, | | WO2017004254 |
| | | 99 in | | |
| | | WO2017004254 | | |
| CD73 | VH | Identifier 100, 103, | VL | Identifier 12. 20, 44, |
| | | 107, 109, 112, 114, | | 72, 76, 8, 84, 92 in |
| | | 116, 119, 121, 16, | | US20160145350; |
| | | 32, 4, 52, 60, 68, 80, | | Identifier 22, 29, 37, |
| | | 88 in | | 4 in |
| | | US20160145350; | | WO2016055609A1; |
| | | Identifier 135, 40, 21, | | Identifier 101, 102, |
| | | 3, 28, 36 in | | 104, 106, 110, 117, |
| | | WO2016055609A1 | | 118, 120, 122 in |
| | | | | US20160145350 |
| Claudin | VH | Identifier 101, 103, | VL | Identifier 114, 116, |
| | | 105, 107, 109, 111, | | 118, 120, 22, 25, 29, |
| | | 113, 115, 117, 119, | | 33, 37, 41, 45, 49, 53, |
| | | 121, 122, 123, 124, | | 57, 61, 65, 69, 73, 77 |
| | | 125, 126, 127, 128, | | in WO2016073649A1 |
| | | 129, 130, 131, 132, | | |
| | | 133, 134, 135, 136, | | |
| | | 137, 138, 139, 140, | | |
| | | 141, 142, 143, 144, | | |
| | | 145, 146, 147, 148, | | |
| | | 149, 150, 23, 27, 31, | | |
| | | 35, 39, 43, 47, 51, 55, | | |
| | | 59, 63, 67, 71, 75, 79, | | |
| | | 81, 83, 85, 87, 89, 91, | | |
| | | 93, 95, 97, 99 in | | |
| | | WO2016073649A1 | | |
| CLL3 | VH | Identifier 101, 103, | VL | Identifier 100, 102, |
| | | 105, 107, 109, 111, | | 104, 106, 108, 110, |
| | | 113, 115, 117, 119, | | 112, 114, 116, 118, |
| | | 121, 123, 125, 127, | | 120, 122, 124, 126, |
| | | 129, 131, 133, 135, | | 128, 130, 132, 134, |
| | | 137, 139, 141, 145, | | 136, 138, 140, 144, |
| | | 147, 149, 151, 153, | | 146, 148, 150, 152, |
| | | 155, 157, 159, 161, | | 154, 156, 158, 160, |
| | | 163, 165, 167, 169, | | 162, 164, 166, 170, |
| | | 171, 173, 175, 177, | | 172, 174, 176, 178, |
| | | 179, 181, 183, 185, | | 180, 182, 184, 186, |
| | | 187, 191, 193, 195, | | 190, 192, 194, 196, |
| | | 197, 199, 201, 203, | | 198, 20, 200, 202, |
| | | 205, 207, 209, 21, | | 204, 206, 208, 210, |
| | | 211, 213, 23, 25, 27, | | 212, 22, 24, 26, 28, |
| | | 29, 31, 33, 35, 37, 39, | | 30, 32, 34, 36, 38, 40, |
| | | 41, 43, 45, 47, 49, 51, | | 42, 44, 46, 48, 50, 54, |
| | | 53, 55, 57, 59, 61, 63, | | 56, 58, 60, 62, 64, 66, |
| | | 65, 67, 69, 71, 73, 75, | | 68, 70, 72, 74, 76, 78, |
| | | 77, 79, 81, 83, 85, 87, | | 80, 82, 84, 86, 88, 90, |
| | | 89, 91, 93, 95, 97, 99 | | 92, 94, 96, 98 in |
| | | in US20170000901 | | US20170000901 |
| OX40 | VH | Identifier 101, 103, | VL | Identifier 10, 45, 47, |
| | | 105, 107, 109, 111, | | 49, 8 in |
| | | 113, 115, 117, 119, | | US8283450; |
| | | 121, 123, 124, 125, | | Identifier 11, 7 in |
| | | 17, 28, 318, 37, 48, | | US9428570; |
| | | 50, 58, 66,, 74, 85, | | Identifier 116, 120, |
| | | 93, 95, 97, 99 in | | 122, 30, 38, 49, 57, |
| | | WO2016196228; | | 65, 73, 84, 86, 94, 98 |
| | | Identifier 31, 34, 36, | | in WO2016196228; |
| | | 38, 40, 42, 44, 46, | | Identifier 24, 26, 27, |
| | | 48, 50, 53, 54, 55, | | 28, 30, 60, 8, 81, 82, |
| | | 58, 59, 61 in | | 83, 84, 85, 86, 87, 88, |
| | | US20150190526; | | 89 in US8748585; |
| | | Identifier 33, 35, 37, | | Identifier 30, 32 in |
| | | 39, 41, 43, 45, 47, | | US20160137740; |
| | | 49, 51, 53, 55, 57, | | Identifier 32, 35, 39, |
| | | 59, 61, 63, 65, 67, | | 41, 43, 45, 47, 49, 51, |
| | | 71 in | | 52, 56, 57, 62 in |
| | | US20160137740; | | US20150190506; |
| | | Identifier 44, 46, 48, | | Identifier 29, 37 in |
| | | 7, 9 in | | US20160137740 |
| | | US8283450; | | |
| | | Identifier 9, 15 in | | |
| | | US9428570; | | |
| | | Identifier 19, 21, 22, | | |
| | | 23, 29, 58, 59, 7, 77, | | |
| | | 78, 79, 80 in | | |
| | | US8748585 | | |
| MUCIN1 | VH | Identifier 101, 106, | VL | Identifier 148, 158, |
| | | 109, 115, 119, 123, | | 162, 167, 170, 174, |
| | | 127, 141, 15, 23, 28, | | 184, 190, 193, 203, |
| | | 33, 39, 42, 47, 5, 57, | | 208, 211, 220, 225, |
| | | 66, 70, 75, 80, 83, | | 229, 234, 242, 246, |
| | | 87, 92 in | | 250, 255, 261, 270, |
| | | EP3049812A2 | | 275, 279, 283, 291, |
| | | | | 297, 303, 308, 315, |
| | | | | 319, 323, 333, 340 |
| | | | | in EP3049812A2 |
| MCSF | VH | Identifier 102, 10, 14, | VL | Identifier 8, 32, 52, |
| | | 18, 2, 22, 26, 30, 34, | | 60, 28, 36, 4, 44, 48, |
| | | 38, 46, 50, 54, 58, 6, | | 56, 62, 12, 16, 20, 24 |
| | | 66, 70, 74, 78, 82, | | in WO2005030124 |
| | | 86, 90, 94, 98 in | | |
| | | WO2005030124 | | |
| LAG3 | VH | Identifier 102, 106, | VL | Identifier 32, 36, 40, |
| | | 110, 113, 122, 18, 30, | | 44, 48, 52, 56, 60, 84, |
| | | 66, 70, 74, 78 in | | 88, 92, 96, 134, 34, |
| | | US20150259420; | | 38, 42, 46, 50, 54, 58, |
| | | Identifier 100, 104, | | 60, 86, 90, 94, 98 in |
| | | 108, 28, 64, 68, 72, | | US20150259420; |
| | | 76, 8, 80 in | | Identifier 2 in |
| | | US20150259420; | | WO2015042246 |
| | | Identifier 1 in | | |
| | | WO2015042246 | | |
| TIM3 | VH | Identifier 102, 112, | — | — |
| | | 12, 2, 22, 32, 42, 52, | | |
| | | 62, 72, 82, 91 in | | |
| | | US20150086574; | | |
| | | Identifier 82 in | | |
| | | WO2013006490; | | |
| | | Identifier 13, 21, 29, | | |
| | | 37, 45, 5, 53, 61, 69, | | |
| | | 77, 85, 93 in | | |
| | | WO2016179319A1; | | |
| | | Identifier 7 in | | |
| | | WO2013006490; | | |
| | | Identifier 107, 117, | | |
| | | 17, 27, 37, 47, 57, | | |
| | | 67, 7, 77, 87, 97 in | | |
| | | US20150086574; | | |
| | | Identifier 17, 25, 33, | | |
| | | 41, 49, 57, 65, 73, | | |
| | | 81, 89, 9, 97 in | | |
| | | WO2016179319A1 | | |
| CD2 | VH | Identifier 103, 117, | VL | Identifier 102, 116 in |
| | | 119 in | | WO2016122701 |
| | | WO2016122701 | | |
| CD52 | VH | Identifier 103, 136, | VL | Identifier 102, 138 |
| | | 137 in | | in WO2010132659 |
| | | WO2010132659 | | |
| WT1/HLA | VH | Identifier 104, 111, | VL | Identifier 106, 112, |
| Bispecific | | 128, 14, 32, 50, 68, | | 130, 34, 52, 70, 88 in |
| | | 86 in | | WO2015070061 |
| | | WO2015070061 | | |
| CD3 | VH | Identifier 108, 112, | VL | Identifier 104 in |
| | | 115 in | | WO2016122701; |
| | | WO2016122701; | | Identifier 13 in |
| | | Identifier 29 in | | WO2016126213A1 |
| | | WO2014144722 A2; | | |
| | | Identifier 12 in | | |
| | | WO2016126213A1 | | |
| PG9 | VH | Identifier 11 in | VL | Identifier 10 in |
| | | EP3074419A2 | | EP3074419A2 |
| V2 | VH | Identifier 11 in | — | — |
| | | US20160194375A1 | | |
| HSP | VH | Identifier 11, 12 in | VL | Identifier 16, 17 in |
| | | WO2016120217 | | WO2016120217 |
| CD37 | VH | Identifier 11, 12, 18 | VL | Identifier 14, 15 in |
| | | in US20170000900 | | US20170000900 |
| CD123 | VH | Identifier 11, 13, 14, | VL | Identifier 9, 11, 18, |
| | | 21 in | | 19, 20, 21, 22, 23 in |
| | | WO2015140268A1; | | WO2016120220; |
| | | Identifier 113, 115, | | Identifier 12, 16, 18, |
| | | 57, 59, 63 in | | 19, 22 in |
| | | WO2016120216; | | WO2015140268A1; |
| | | Identifier 12, 123, | | Identifier 275, 276, |
| | | 24, 25, 26, 27, 28, 29, | | 277, 278, 307, 308, |
| | | 30, 9 in | | 309, 310 in |
| | | WO20161620220; | | WO2016028896; |
| | | Identifier 216, 217, | | Identifier 5 in |
| | | 218, 219, 274 in | | US20160333108A1; |
| | | WO2016028896 | | Identifier 114, 116, |
| | | | | 58, 60, 64 in |
| | | | | WO2016120216 |
| GD3 | VH | Identifier 11, 13, 15, | VL | Identifier 12, 14, 16, |
| | | 17 in | | 18 in |
| | | WO2016185035A1 | | WO2016185035A1 |
| TAG72 | VH | Identifier 115 in | VL | Identifier 116 in |
| | | US20160333114A1 | | US20160333114A1 |
| MCAM | VH | Identifier 115, 116, | VL | Identifier 109, 110, |
| | | 117, 118, 119, 157, | | 111, 112, 121, 122, |
| | | 158, 159, 160, 161, | | 123 in |
| | | 178, 179 in | | US20150259419; |
| | | US20150259419; | | Identifier 30, 40, 50, |
| | | Identifier 35, 45, 55, | | 60, 70, 71, 72 in |
| | | 65, 77, 89 in | | US20150239980 |
| | | US20150239980; | | |
| | | Identifier 101, 102, | | |
| | | 103, 104, 105, 106, | | |
| | | 107 in | | |
| | | US20150259419 | | |
| CA19.9 | VH | Identifier 117 in | VL | Identifier 118 in |
| | | US20160333114A1 | | US20160333114A1 |
| BMPR1A | VH | Identifier 12 in | — | — |
| | | WO2011116212 | | |
| LGR4 | VH | Identifier 12, 13, 5, | VL | Identifier 10, 11, 6 in |
| | | 9 in | | US20160046723 |
| | | US20160046723 | | |
| APRIL | VH | Identifier 12, 14, 16. | VL | Identifier 20, 22, 24, |
| | | 18. 3. 32, 34, 36, 38, | | 26, 28, 30, 4, 50 in |
| | | 40, 42, 44, 46, 48, | | US2016026467 |
| | | 52 in | | |
| | | US20160264674 | | |
| FcRL5 | VH | Identifier 12, 16, 20, | VL | Identifier 11, 15, 19, |
| (FcReceptor- | | 24, 28, 32, 36, 4, 40, | | 23, 27, 3, 31, 35, 39, |
| Like5) | | 44, 48, 8, 915, 919 | | 43, 47, 7, 917, 921 |
| | | in WO2016090337 | | WO2016090337 |
| CLDN18.2 | VH | Identifier 12, 2 in | VL | Identifier 13, 3 in |
| | | US20160347915A1 | | US20160347815A1 |
| ROR1 | VH | Identifier 12, 20, 28, | VL | Identifier 16, 24, 32, |
| | | 36, 44, 60, 68 | | 40, 56, 64, 72, 36, 62, |
| | | WO2016016343A1; | | 23, 49, 58 |
| | | Identifier 57, 19, 31, | | WO2016016343A1; |
| | | 45, 53, 71 in | | Identifier 86, 88, 90 in |
| | | WO2016016344A1; | | WO2016120216; |
| | | Identifier 85, 87, 89 | | Identifier 126, 127, |
| | | in WO2016120216; | | 234, 235, 236, 237, |
| | | Identifier 122, 125, | | 238, 240, 241, 242, |
| | | 175, 176, 179, 180, | | 243, 244, 245, 246, |
| | | 181, 182, 183, 184, | | 247, 248 in |
| | | 185, 186, 187, 188, | | US20160208018A1; |
| | | 189, 190, 191, 192, | | Identifier 56 in |
| | | 193, 194, 195, 196, | | EP3083671A1; |
| | | 197, 197, 199, 200, | | Identifier 103, 111, |
| | | 201, 202, 203, 204, | | 127, 135, 143, 15, 151, |
| | | 205, 206, 207, 208, | | 159, 167, 175, 183, |
| | | 209 in | | 191, 199, 207, 215, |
| | | US20160208018A1; | | 223, 23, 231, 239, |
| | | Identifier 55 in | | 247, 255, 263, 271, |
| | | EP3083671A1; | | 279, 287, 295, 303, |
| | | Identifier 104, 112, | | 31, 311, 319, 327, 335, |
| | | 120, 128, 152, 16, | | 343, 351, 359, 39, 47, |
| | | 160, 168, 176, 184, | | 55, 53, 7, 71, 79, 87, |
| | | 192, 200, 208, 216, | | 95 in |
| | | 224, 232, 24, 240, | | WO2016187216A1 |
| | | 248, 256, 264, 272, | | |
| | | 280, 288, 296, 304, | | |
| | | 312, 32, 320, 336, | | |
| | | 344, 352, 360, 40, | | |
| | | 48, 56, 64, 72, 8, 80, | | |
| | | 88 in | | |
| | | WO2016187216A1 | | |
| B7H1 | VH | Identifier 12, 32, 42, | VL | Identifier 17, 37, 47, |
| | | 52, 72, 2, 62 in | | 57, 7, 77, 27, 67 in |
| | | US20130034559 | | US20130034559 |
| CD32B | VH | Identifier 127 in | VL | Identifier 126 in |
| | | WO2016122701 | | WO2016122701 |
| CD64 | VH | Identifier 129 in | VL | Identifier 128 in |
| | | WO2016122701 | | WO2016122701 |
| PG16 | VH | Identifier 13 in | VL | Identifier 12 in |
| | | EP3074419A2 | | EP3074419A2 |
| MPER | VH | Identifier 13 in | VL | Identifier 12 in |
| | | US20160194375A1 | | US20160194375A1 |
| CD105 | VH | Identifier 13, 14, 16 | VL | Identifier 1, 17, 20, |
| | | in WO2014039682 | | 22, 23 in |
| | | | | WO2014039682 |
| CLL1 | VH | Identifier 13, 14, 15, | VL | Identifier 16, 18, 20, |
| | | 17, 19, 21, 23, 25, | | 22, 24, 26, 28, 30, 32, |
| | | 27, 29, 31, 33, 35 in | | 24, 36 in |
| | | WO2016120219; | | WO2016120219; |
| | | Identifier 195, 65, | | Identifier 196, 78, 79, |
| | | 66, 67, 68, 69, 70, | | 80, 81, 82, 83, 84, 85, |
| | | 71, 72, 73, 74, 75, | | 86, 87, 88, 89, 90 in |
| | | 76, 77 in | | WO2016014535; |
| | | WO2016014535; | | Identifier 30, 32, 35, |
| | | Identifier 31, 33, 34, | | 37, 39, 41 in |
| | | 36, 38, 40, 42, 46 in | | US20160075787; |
| | | US20160075787; | | Identifier 152, 104, |
| | | Identifier 150, 103, | | 106, 108, 110, 112, |
| | | 105, 107, 109, 111, | | 114, 116, 118 in |
| | | 113, 115, 117 in | | WO2016179319A1 |
| | | WO2016179319A1 | | |
| GPRC5D | VH | Identifier 13, 17, 21, | VL | Identifier 10, 14, 18, 2, |
| | | 25, 29, 314, 326, 33, | | 22, 26, 30, 303, 315, |
| | | 338, 350, 362, 37, | | 327, 339, 34, 351, 363, |
| | | 374, 386, 41, 45, 49, | | 375, 38, 387, 42, 46, |
| | | 5, 53, 57, 61, 65, 69, | | 50, 54, 58, 6, 62, 66, |
| | | 73, 77, 81, 85, 89, 93, | | 70, 74, 78, 82, 86, 94 |
| | | 1, 9 in | | in WO2016090312 |
| | | WO2016090312 | | |
| EFNA4 | VH | Identifier 13, 39 in | — | — |
| | | US20150125472 | | |
| CD79 | VH | Identifier 131 in | VL | Identifiier 130 in |
| | | WO2016122701 | | WO2016122701 |
| FGFR3 | VH | Identifier 132, 134, | VL | Identifier 133, 135, |
| | | 136 in US9499623 | | 137 in US9499623 |
| TCR | VH | Identifier 133 in | VL | Identifier 132 in |
| | | WO2016122701 | | WO2016122701 |
| MN | VH | Identifier 133, 135, | VL | Identifier 134, 136, |
| | | 137, 139, 141, 143, | | 138, 140, 142, 144, |
| | | 145, 147, 149, 151 in | | 146, 148, 150, 152 in |
| | | WO2007070538 | | WO200707538 |
| IL33 | VH | Identifier 134, 136, | VL | Identifier 135, 137, |
| | | 138, 185, 187, 189, | | 139, 184, 188, 217, |
| | | 216, 218, 220, 221, | | 219, 237, 247, 283, |
| | | 236, 246, 282, 284, | | 285, 287, 37, 39, 41, |
| | | 286, 36, 38, 40, 84, | | 87 in |
| | | 86, 88 in | | US20160168242 |
| | | US20160168242 | | |
| NKG2D | VH | Identifier 135, 137 | VL | Identifier 134, 136 in |
| | | in WO2016122701 | | WO2016122701 |
| CD30 | VH | Identifier 14, 16 in | VL | Identifier 13, 15 in |
| | | WO2016134284 | | WO2016134284 |
| EGFR | VH | Identifier 14, 50, 9 | VL | Identifier 15 in |
| | | in WO2015143382; | | WO2015143382; |
| | | Identifier 12, 14, 15, | | Identifier 14 in |
| | | 21 in | | WO2014143765; |
| | | US20100008978A1; | | Identifier 4051, 4052, |
| | | Identifier 2123 in | | 4053, 4054, 4055, |
| | | WO2018231759 | | 4056, 4057, 4058, |
| | | | | 4059, 4060, 4061, |
| | | | | 4062, 4063, 4064, |
| | | | | 4065, 4066, 4067, |
| | | | | 4068, 4069, 4070, |
| | | | | 4071, 4072, 4073, |
| | | | | 4074, 4075, 4076, |
| | | | | 4077, 4078, 4079, |
| | | | | 4080, 4081, 4082, |
| | | | | 4083, 4084, 4085, |
| | | | | 4086, 4087, 4088, |
| | | | | 4089. 4090, 4091, |
| | | | | 4092, 4093, 4094, |
| | | | | 4095, 4096, 4097, |
| | | | | 4098, 4099, 4100, |
| | | | | 4101, 4102, 4103, |
| | | | | 4104, 4105, 4106, |
| | | | | 4107, 4108, 4109, |
| | | | | 4110, 4111, 4112, |
| | | | | 4113, 4114, 4115, |
| | | | | 4116, 4117, 4118, |
| | | | | 4119, 4120, 4121, |
| | | | | 4122, 4123, 4124, |
| | | | | 4125, 4126, 4127, |
| | | | | 4128, 4129, 4130, |
| | | | | 4131, 4132, 4133, |
| | | | | 4134, 4135, 4136, |
| | | | | 4137, 4138, 4139, |
| | | | | 4140, 4141, 4142, |
| | | | | 4143, 4144, 4145, |
| | | | | 4156, 4147, 4148, |
| | | | | 4149, 4150, 4151, |
| | | | | 4152, 4153, 4154, |
| | | | | 4155, 4156, 4157, |
| | | | | 4158, 4159, 4160, |
| | | | | 4161, 4162, 4163, |
| | | | | 4164, 4165, 4166, |
| | | | | 4167, 4168, 4169, |
| | | | | 4170, 4171, 4172, |
| | | | | 4173, 4174, 4175, |
| | | | | 4176, 4177, 4178, |
| | | | | 4179, 4180, 4181, |
| | | | | 4182, 4183, 4184, |
| | | | | 4185, 4186, 4187, |
| | | | | 4188, 4189, 4190, |
| | | | | 4191, 4192, 4193, |
| | | | | 4194, 4195, 4196, |
| | | | | 4197, 4198, 4199, |
| | | | | 4200, 4201, 4202, |
| | | | | 4203, 4204, 4205, |
| | | | | 4206, 4207, 4208, |
| | | | | 4209, 4210, 4211, |
| | | | | 4212, 4213, 4214, |
| | | | | 4215, 4216, 4217, |
| | | | | 4218, 4219, 4220, |
| | | | | 4221, 4222, 4223, |
| | | | | 4224, 4225, 4226, |
| | | | | 4227, 4228, 4229, |
| | | | | 4230, 4231, 4232, |
| | | | | 4233, 4234, 4235, |
| | | | | 4236, 4237, 4238, |
| | | | | 4239, 4240, 4241, |
| | | | | 4242, 4243, 4244, |
| | | | | 4245, 4246, 4247, |
| | | | | 4248, 4249, 4250, |
| | | | | 4251, 4252, 4253, |
| | | | | 4254, 4255, 4256, |
| | | | | 4257, 4258, 4259, |
| | | | | 4260, 4261, 4262, |
| | | | | 4263, 4264, 4265, |
| | | | | 4266, 4267, 4268, |
| | | | | 4269, 4270, 4271, |
| | | | | 4272, 4273, 4274, |
| | | | | 4275, 4276, 4277, |
| | | | | 4278, 4279, 42870, |
| | | | | 4281, 4282, 42834284, |
| | | | | 4285, 4286, 4287, |
| | | | | 4288, 4289, 4290, |
| | | | | 4291, 4292, 4293, |
| | | | | 4294, 4295, 4296, |
| | | | | 4297, 4298, 4299, |
| | | | | 4300, 4301, 4302, |
| | | | | 4303, 4304, 4305, |
| | | | | 4306, 4307, 4308, |
| | | | | 4309, 4310, 4311, |
| | | | | 4312, 4313, 4314, |
| | | | | 4315, 4316, 4317, |
| | | | | 4318, 4319, 4320, |
| | | | | 4321, 4322, 4324, |
| | | | | 4325, 4326, 4327, |
| | | | | 4328, 4329, 4330, |
| | | | | 4331, 4332, 4333, |
| | | | | 4334, 4335, 4336, |
| | | | | 4337, 4338, 4339, |
| | | | | 4340, 4341, 4342, |
| | | | | 4343, 4344, 4345, |
| | | | | 4346, 4347, 4348, |
| | | | | 4349, 4350, 4351, |
| | | | | 4352, 4353, 4354, |
| | | | | 4355, 4356, 4357, |
| | | | | 4358, 4359, 4360, |
| | | | | 4361, 4362, 4363, |
| | | | | 4364, 4365, 4366, |
| | | | | 4367, 4368, 4369, |
| | | | | 4370, 4371, 4372, |
| | | | | 4373, 4374, 4375, |
| | | | | 4376, 4377, 4378, |
| | | | | 4379, 4380, 4381, |
| | | | | 4382, 4383, 4384, |
| | | | | 4385, 4386, 4387, |
| | | | | 4388, 4389, 4390, |
| | | | | 4391, 4392, 4393, |
| | | | | 4394, 4395, 4396, |
| | | | | 4397, 4398, 4399, |
| | | | | 4400, 4401, 4402, |
| | | | | 4403, 4404, 4405, |
| | | | | 4406, 4407, 4408, |
| | | | | 4409, 4410, 4411, |
| | | | | 4412, 4413, 4414, |
| | | | | 4415, 4416, 4417, |
| | | | | 4418, 4419, 4420, |
| | | | | 4421, 4422, 4423, |
| | | | | 4424, 4425, 4426, |
| | | | | 4427, 4428, 4429, |
| | | | | 4430, 4431, 4432, |
| | | | | 4433, 4434, 4435, |
| | | | | 4436, 4437, 4438, |
| | | | | 4439, 4440, 4441, |
| | | | | 4442, 4443, 4444, |
| | | | | 4445, 4446, 4447, |
| | | | | 4448, 4449, 4450, |
| | | | | 4451, 4452, 4453, |
| | | | | 4454, 4455, 4456, |
| | | | | 4457, 4458, 4459, |
| | | | | 4460, 4461, 4462, |
| | | | | 4463, 4464, 4465, |
| | | | | 4466, 4467, 4468, |
| | | | | 4469, 4470, 4471, |
| | | | | 4472, 4473, 4474, |
| | | | | 4475, 4476, 4477, |
| | | | | 4478, 4479, 4480, |
| | | | | 4481, 4482, 4483, |
| | | | | 4484, 4485, 4486, |
| | | | | 4487, 4488, 4489, |
| | | | | 4490, 4491, 4492, |
| | | | | 4493, 4494, 4495, |
| | | | | 4496, 4497, 4498, |
| | | | | 4499, 4500, 4501, |
| | | | | 4502, 4503, 4504, |
| | | | | 4505, 4506, 4507, |
| | | | | 4508, 4509, 4510, |
| | | | | 4511, 4512, 4513, |
| | | | | 4514, 4515, 4516, |
| | | | | 4517, 4518, 4519, |
| | | | | 4520, 4521, 4522, |
| | | | | 4523, 4524, 4525, |
| | | | | 4526, 4527, 4528, |
| | | | | 4529, 4530, 4531, |
| | | | | 4532, 4533, 4534, |
| | | | | 4535, 4536, 4537, |
| | | | | 4538, 4539, 4540, |
| | | | | 4541, 4542, 4543, |
| | | | | 4544, 4545, 4546, |
| | | | | 4547, 4548, 4549, |
| | | | | 4550, 4551, 4552, |
| | | | | 4553, 4554, 4555, |
| | | | | 4556, 4557, 4558, |
| | | | | 4559, 4560, 4561, |
| | | | | 4562, 4563, 4564, |
| | | | | 4565, 4566, 4567, |
| | | | | 4568, 4569, 4570, |
| | | | | 4571, 4572, 4573, |
| | | | | 4574, 4575, 4576, |
| | | | | 4577, 4578, 4579, |
| | | | | 4580, 4581, 4582, |
| | | | | 4583, 4584, 4585, |
| | | | | 4586, 4587, 4588, |
| | | | | 4589, 4590, 4591, |
| | | | | 4592, 4593, 4594, |
| | | | | 4595, 4596, 4597, |
| | | | | 4598, 4599, 4600, |
| | | | | 4601, 4602, 4603, |
| | | | | 4604, 4605, 4606, |
| | | | | 4607, 4608, 4609, |
| | | | | 4610, 4611, 4612, |
| | | | | 4613, 4614, 4615, |
| | | | | 4616, 4617, 4618, |
| | | | | 4619, 4620, 4621, |
| | | | | 4622, 4623, 4624, |
| | | | | 4625, 4626, 4627, |
| | | | | 4628, 4629, 4630, |
| | | | | 4631, 4632, 4633, |
| | | | | 4634, 4635, 4636, |
| | | | | 4637, 4638, 4639 in |
| | | | | WO2018231759 |
| Her2 | VH | Identifier 141, 262, | VL | Identifier 140 in |
| | | 264, 266, 268, 270 | | WO2016054555A2; |
| | | in WO2016168773A3; | | Identifier 261, 263, |
| | | Identifier 11 in | | 265, 267, 269 in |
| | | US9518118; | | WO2016168773A3; |
| | | Identifier 62 in | | Identifier 10, 18, 23 in |
| | | US20160333114A1; | | US9518118; |
| | | Identifier 19, 24 in | | Identifier 3 in |
| | | US9518118 | | WO2016168769A1; |
| | | | | Identifier 59, 61 in |
| | | | | US20160333114A1 |
| EFNA | VH | Identifier 149, 153, | VL | Identifier 151, 155, |
| | | 157, 161 in | | 159, 163 in |
| | | US20150125472 | | WO2012118547; |
| | | | | Identifier 27, 53 in |
| | | | | US20150125472 |
| PGT1 | VH | Identifier 15 in | — | — |
| | | EP307441 | | |
| Factor XII | VH | Identifier 15 in | VL | Identifier 17 in |
| | | WO2014089493 | | WO2014089493 |
| E7MC | VH | Identifier 15, 16, 17, | VL | Identifier 238, 239, |
| | | 18, 19, 20, 21, 22, 23, | | 240, 241, 242, 243, |
| | | 233, 234, 235, 236, | | 36, 37, 38, 39, 41, 42, |
| | | 237, 24, 25, 26, 27, | | 43, 44, 45, 46, 47, 48, |
| | | 28, 29, 30, 31, 32, 33, | | 49, 50, 51, 52, 53, 54, |
| | | 34, 35 in | | 55, 56 in |
| | | WO2016182957A1 | | WO2016182957A1 |
| ICOS | VH | Identifier 15, 16, 19, | VL | Identifier 17, 18, 20, |
| | | 23, 7 in | | 24, 8 in |
| | | US20160215059 | | US20160215059 |
| CD76b | VH | Identifier 15, 17, 19, | VL | Identifier 16, 18, 22, |
| | | 23, 27, 29, 37, 57, 59, | | 38, 58, 60, 62 in |
| | | 61 in | | US20160159906 |
| | | US20160159906 | | |
| MUC1C ECD | VH | Identifier 15, 19, 23, | VL | Identifier 17, 21, 25, |
| | | 60, 64, 68, 72 in | | 62, 66, 70, 75 in |
| | | US20160340442A1 | | US20160340442A1 |
| CD4BS | VH | Identifier 15, 3 in | VL | Identifier 14, 2 in |
| | | US20160194375A1 | | US20160194375A1 |
| CD7 | VH | Identifier 16, 20 in | VL | Identifier 17, 21 in |
| | | WO2016126213A1 | | WO2016126213A1 |
| PGT2 | VH | Identifier 17 in | VL | Identifier 16 in |
| | | EP3047719A2 | | EP3074419A2 |
| OTK3 | VH | Identifier 17 in | VL | Identifier 18 in |
| | | WO2015158868 | | WO2015158868 |
| GD2 | VH | Identifier 17 in | VL | Identifier 10, 2, 5, 7, |
| | | WO2016134284; | | 9 in |
| | | Identifier 1 in | | US20130216528; |
| | | US20130216528; | | Identifier 11, 12 in |
| | | Identifier 10, 9 in | | WO2015132604; |
| | | WO2015132604; | | Identifier 18 in |
| | | Identifier 3, 4, 6, 8 in | | WO2016134284 |
| | | US20130216528 | | |
| Trophoblast | VH | Identifier 17, 13, 15, | VL | Identifier 18, 12, 14, |
| Glycoprotein | | 11 in | | 16 in |
| 5T4 | | WO2016034666A1 | | WO2016034666A1 |
| AMC | VH | Identifier 17, 18, 19, | VL | Identifier 27, 28, 29, |
| | | 20, 21, 22, 23, 24, 25, | | 31, 32, 33, 34, 35, 36 |
| | | 26 in | | in WO2016161390 |
| | | WO2016161390 | | |
| Factor D | VH | Identifier 17, 20, 27, | VL | Identifier 16, 18, 19, |
| | | 29, 30, 31, 32, 33, 4 | | 26, 3 in |
| | | in US20160017052 | | US20160017052 |
| CD276 | VH | Identifier 17, 26, 7 in | VL | Identifier 18, 27 in |
| | | US20160053017 | | US20160053017 |
| B7H3 | VH | Identifier 17, 26, 7 in | VL | Identifier 18, 27, 8 in |
| (CD276) | | WO2016044383 | | WO2016044383 |
| RAS | VH | Identifier 17, 47, 57, | VL | Identifier 19, 49, 59, |
| | | 67, 7, 77 in | | 69, 79, 9 in |
| | | WO2016154047 | | WO2016154047 |
| DR5 | VH | Identifier 18, 82, 90, | VL | Identifier 13, 23, 25, |
| | | 98, 8 in | | 27, 3, 78, 86, 94, 29 in |
| | | WO2016122701 | | WO2016122701; |
| | | | | Identifier 62 in |
| | | | | WO2016122701; |
| | | | | Identifier 54 in |
| | | | | WO2016122701; |
| | | | | Identifier 70 in |
| | | | | WO2016122701 |
| PGT3 | VH | Identifier 19 in | VL | Identifier 18 in |
| | | EP3074419A2 | | EP3074419A2 |
| PD1 | VH | Identifier 19 in | VL | Identifier 2, 39, 7, 8, |
| | | US20150290316; | | 9 in |
| | | Identifier 25, 26, 27, | | US 20160159905; |
| | | 28, 29 in | | Identifier 21 in |
| | | US20130291136; | | US20150290316; |
| | | Identifier 29, 3, 38 in | | Identifier 30, 31, 32, |
| | | US20160159905; | | 33 in |
| | | Identifier 38, 50 in | | US20130291136; |
| | | WO2015112900; | | Identifier 42, 46, 54 in |
| | | Identifier 4, 4, 6 in | | WO2015112900; |
| | | US20160159905; | | Identifier 58, 62, 66, |
| | | Identifier 82, 86 in | | 70, 74, 78 in |
| | | WO2015112900; | | WO2015112900; |
| | | Identifier 17 in | | Identifier 18 in |
| | | WO2014055648; | | WO2014055648; |
| | | Identifier 4 in | | Identifier 5 in |
| | | WO2016040892; | | WO2016040892; |
| | | Identifier 12 in | | Identifier 13 in |
| | | US20150190506 | | US20150190506 |
| NYBR1 | VH | Identifier 19 in | VL | Identifier 18 in |
| | | US20160333422A1 | | US20160333422A1 |
| CD28 | VH | Identifier 19 in | VL | Identifier 20 in |
| | | WO2015158868 | | WO2015158868 |
| Tn | VH | Identifier 19, 20 in | — | — |
| Glycopeptide | | WO2015120180 | | |
| human- | VH | Identifier 19, 29, 38, | | |
| ERBB3 | | 45, 55, 61, 9 in | VL | Identifier 10, 20, 30, |
| | | WO2013052745 | | 39, 46, 56, 62 in |
| | | | | WO2013052745 |
| Olfml3 | VH | Identifier 19, 3 in | VL | Identifier 20, 4 in |
| | | WO2015054441A1 | | WO2015054441A1 |
| RHAMM | VH | Identifier 2 in | VL | Identifier 4 in |
| antagonist | | WO2000029447 | | WO2000029447 |
| body | | | | |
| CD38 | VH | Identifier 2 in | VL | Identifier 1, 11 in |
| | | WO2009080830; | | WO2009080830 |
| | | Identifier 10 in | | |
| | | WO2015121454 | | |
| PDl | VH | Identifier 2 in | VL | Identifier 11 in |
| (Nivolumab) | | WO2016040892; | | US20150190506 |
| | | Identifier 10 in | | |
| | | US20150190506 | | |
| PDK1 | vH | Identifier 2 in | VL | Identifier 9 in |
| | | WO2016090365 | | WO2016090365 |
| IL21 | VH | Identifier 2, 3 in | — | — |
| | | US20160145332 | | |
| ERBB2 | VH | Identifier 2, 4 in | VL | Identifier 1 in |
| | | US20110129464; | | US20110129464; |
| | | Identifier 10, 2, 26, | | Identifier 12, 16, 20, |
| | | 30, 38, 4, 40, 42, 52, | | 24, 32, 36, 44, |
| | | 54, 56, 57, 58, 6 in | | 50, 51, 53, 8 in |
| | | US20130089544; | | US20130089544; |
| | | Identifier 8 in | | Identifier 7 in |
| | | US20130266564; | | US20130266564; |
| | | Identifier 1 in | | Identifier 3 in |
| | | US20150104443 | | US20150104443 |
| 5T4 | VH | Identifier 2, 4 in | VL | Identifier 1, 3 in |
| | | WO2016022939 | | WO2016022939 |
| GPDL1 | VH | Identifier 20 in | — | — |
| | | US20160108123 | | |
| GM2 | VH | Identifier 20, 22, 23, | VL | Identifier 21, 24, 25, |
| | | 26, 27, 28, 29, 30 in | | 31, 32, 33, 34, 35 in |
| | | US20090028877 | | US20090028877 |
| EphA2 | VH | Identifier 20, 22, 24, | VL | Identifier 26, 28, 30, |
| receptor | | 32, 34, 36, 37, 38, 40, | | 47, 48, 49, 50, 52, 78, |
| | | 42, 43, 45, 74, 76 in | | 80 in |
| | | US20150274824 | | US20150274824 |
| KDR | VH | Identifier 20, 24, 26, | VL | Identifier 22 in |
| | | 29, 31, 33 in | | WO2003075840 |
| | | WO2003075840 | | |
| PGT4 | VH | Identifier 21 in | VL | Identifier 20 in |
| | | EP3074419A2 | | EP3074419A2 |
| CD324 | VH | Identifier 21, 23, 25, | VL | Identifier 20, 22, 24 |
| | | 27, 29, 31, 33, 35, 37, | | 26, 28, 30, 32, 34, 36, |
| | | 39, 41, 43, 45, 47, 49, | | 38, 40, 42, 44, 46, 48, |
| | | 51, 53, 55, 57, 59, 61, | | 50, 52, 54, 56, 58, 60, |
| | | 63, 65, 67, 69, 71 in | | 62, 64, 66, 68, 70 in |
| | | US9534058 | | US9534058 |
| AXL | VH | Identifier 21, 3, 45 in | VL | Identifier 22, 4 in |
| | | WO2016097370 | | WO2016097370 |
| collagen | VH | Identifier 21, 4, 15, 17, | VL | Identifier 11, 12, 14, |
| | | 18, 19, 20, 5, 6, 7, 1, | | 23, 25, 26, 27, 8, 9 in |
| | | 2, 3 in | | WO2007024921 |
| | | WO2007024921 | | |
| EGFR | VH | Identifier 2124 in | VL | 4640, 4641, 4642, |
| (EGFRvIII) | | WO2018231759; | | 4643, 4644, 4645, |
| | | Identifier 13 in | | 4646, 4647, 4648, |
| | | WO2016016341; | | 4649, 4650, 4651, |
| | | Identifier 24 in | | 4652, 4653, 4654, |
| | | WO2016168773 A3; | | 4655, 4656, 4657, |
| | | Identifier 34 in | | 4658 in |
| | | US20160304615; | | WO2018231759; |
| | | Identifier 2 in | | Identifier 14 in |
| | | US20160200819A1; | | WO2016016341; |
| | | Identifier 91, 93 in | | Identifier 23 in |
| | | WO2016120216 | | WO2016168773 A3; |
| | | | | Identifier 42 in |
| | | | | US20160304615; |
| | | | | Identifier 1 in |
| | | | | US20160200819A1; |
| | | | | Identifier 92, 94 in |
| | | | | WO2016120216 |
| IL3alpha | VH | Identifier 22 in | VL | Identifier 27, 37 in |
| | | WO2008127735 | | WO2008127735 |
| CS1 | VH | Identifier 22 in | VL | Identifier 104, 106, |
| | | WO2016168773 A3; | | 108 in |
| | | 103, 105, 107, 109, | | WO2016120216; |
| | | in WO2016120216; | | Identifier 14, 16, 18, |
| | | Identifier 13, 15, 17, | | 20, 22 in |
| | | 19 in | | WO2015166056A1; |
| | | WO2015166056A1; | | Identifier 39, 41, 43, |
| | | Identifier 38, 40, 42, | | 45, 47 in |
| | | 44, 46 in | | WO2015121454; |
| | | WO2015121454; | | Identifier 110, 112 in |
| | | Identifier 26 in | | WO2016120216 |
| | | US20160075784A1 | | |
| KMA | VH | Identifier 22 in | VL | Identifier 2, 21 in |
| | | WO2016172703 A2 | | WO2016172703A2 |
| PGT5 | VH | Identifier 23 in | VL | Identifier 22 in |
| | | EP3074419A2 | | EP3074419A2 |
| CD45 | VH | Identifier 24 in | VL | Identifier 25 in |
| | | WO2016126213A1 | | WO2016126213A1 |
| leuko- | VH | Identifier 25 in | — | — |
| cytegenA2 | | WO2010065962 A2 | | |
| CD16 | VH | Identifier 25 in | VL | Identifier 26 in |
| | | WO2015158868 | | WO2015158868 |
| BCMA | VH | Identifier 26 in | VL | Identifier 25 in |
| | | WO2016168773 A3; | | WO2016168773 A3; |
| | | Identifier 142, 148, | | Identifier 42 in |
| | | 154, 160, 166, 172, | | WO2016097231; |
| | | 178, 184, 190, 196, | | Identifier 143, 149, |
| | | 202, 208, 214, 220, | | 155, 161, 167, 173, |
| | | 226, 232, 238, 244, | | 179, 185, 191, 197, |
| | | 250, 256, 262, 268, | | 203, 209, 215, 221, |
| | | 274, 280, 286, 292, | | 227, 233, 239, 245, |
| | | 298, 304, 310, 316, | | 251, 257, 263, 275, |
| | | 322, 328, 334, 340, | | 281, 287, 293, 299, |
| | | 346, 352 in | | 305, 311, 317, 323, |
| | | WO2016168595A1; | | 329, 335, 341, 347, |
| | | Identifier 8 in | | 353 in |
| | | WO2016094304A3; | | WO2016168595 Al; |
| | | Identifier 171, 172, | | Identifier 192, 193, |
| | | 173, 174, 175, 176, | | 194, 195, 196, 197, |
| | | 177, 178, 179, 180, | | 198, 199, 200, 201, |
| | | 181, 182, 183, 184, | | 204, 205, 207, 208, |
| | | 185, 186, 187, 190, | | 211, 259, 260, 84, 85, |
| | | 255, 257, 258, 69, 70, | | 86, 87, 88, 89, 90, 91, |
| | | 71, 72, 73, 74, 75, 76, | | 92, 93, 94, 95, 96, 97, |
| | | 77, 78, 79, 80, 81 | | 98 in |
| | | WO2016014565; | | WO2016014565; |
| | | Identifier 38 in | | Identifier 53 in |
| | | EP3057994A1; | | WO2016187349A1; |
| | | Identifier 55 in | | Identifier 7 in |
| | | WO2016187349A1; | | WO2016094304 A3; |
| | | Identifier 1, 13, 17, 21, | | Identifier 10, 14, 18, |
| | | 25, 29, 33, 37, 41, 45, | | 2, 22, 26, 30, 34, 38, |
| | | 49, 5, 53, 57, 61, 65, 9 | | 42, 46, 50, 54, 58, 6, |
| | | in WO2016090320; | | 62, 66 in |
| | | Identifier 101, 743, | | WO2016090320; |
| | | 174, 758, 95, 759, | | Identifier 100, 102, |
| | | 97, 760, 99 in | | 175, 96, 98 in |
| | | WO2016120216; | | WO2016120216; |
| | | Identifier 11, 741, | | Identifier 12, 14, 16, |
| | | 17 in | | 18 in |
| | | WO2015158671Al; | | WO201515867A1; |
| | | Identifier 10, 11, 12, | | Identifier 7, 8, 9 in |
| | | 13, 14 in | | WO2016014789; |
| | | WO2016014789; | | Identifier 14 in |
| | | Identifier 15 in | | WO2016168766A1 |
| | | WO2016168766A1 | | |
| humanCD79b | VH | Identifier 27 in | VL | Identifier 28, 30 in |
| | | WO2016112870 | | WO2016112870 |
| | | humanCD79b VH | | |
| | | 2304 Identifier 29 in | | |
| | | WO2016112870 | | |
| B2MG | VH | Identifier 28 in | VL | Identifier 29 in |
| | | WO2016126213A1 | | WO2016126213A1 |
| CD19H 803 | VH | Identifier 28, 29, 32, | — | — |
| | | 33, 34, 35 in | | |
| | | WO2016168773 A3; | | |
| | | Identifier 51 in | | |
| | | WO2016187349A1; | | |
| | | Identifier 20 in | | |
| | | US20160039942; | | |
| | | Identifier 1 in | | |
| | | WO2014184143; | | |
| | | Identifier 5 in | | |
| | | US20160145337Al; | | |
| | | Identifier 166, 167, | | |
| | | 168, 172, 176, 177, | | |
| | | 181, 183, 184, 62 in | | |
| | | US20160152723; | | |
| | | Identifier 15 | | |
| | | US20160319020; | | |
| | | Identifier 17, 33, 34, | | |
| | | 35 in EP3057994Al; | | |
| | | Identifier 62 in | | |
| | | WO2016134284; | | |
| | | Identifier 111, 113 in | | |
| | | US20160333114A1 | | |
| NOTCH 2/3 | VH | Identifier 29 in | VL | Identifier 31 in |
| | | WO2013074596 | | WO2013074596 |
| KIR | VH | Identifier 3 in | VL | Identifier 5 in |
| (Lirilumab) | | US20150290316; | | US20150290316; |
| | | 2371 Identifier 1 in | | Identifier 2 in |
| | | WO2014055648 | | WO2014055648 |
| CD22 | VH | Identifier 3 in | VL | Identifier 17, 8, 14, 15, |
| | | WO2013059593; | | in US20150239974; |
| | | Identifier 10, 11, 12, | | Identifier 7 in |
| | | 7, 9, 8 in | | US20150299317; |
| | | US20150299317; | | Identifier 681 in |
| | | Identifier 201 in | | WO2016164731; |
| | | WO2016164731; | | Identifier 682 in |
| | | CD22 VH 869 | | WO2016164731; |
| | | Identifier 671 in | | Identifier 682 in |
| | | WO2016164731; | | WO2016164731; |
| | | Identifier 672 in | | Identifier 683, 2020 in |
| | | WO2016164731; | | WO2016164731; |
| | | Identifier 673 in | | Identifier 684 in |
| | | WO2016164731; | | WO2016164731; |
| | | Identifier 676 in | | Identifier 685 in |
| | | WO2016164731; | | WO2016164731; |
| | | Identifier 678 in | | Identifier 686 in |
| | | WO2016164731; | | WO2016164731; |
| | | Identifier 679 in | | Identifier 687 in |
| | | WO2016164731; | | WO2016164731; |
| | | Identifier 680 in | | Identifier 688 in |
| | | WO2016164731; | | WO2016164731; |
| | | Identifier 700 in | | Identifier 690 in |
| | | WO2016164731; | | WO2016164731; |
| | | Identifier 701 in | | Identifier 740 in |
| | | WO2016164731; | | WO2016164731; |
| | | Identifier 702 in | | Identifier 741 in |
| | | WO2016164731; | | WO2016164731; |
| | | Identifier 703 in | | Identifier 742 in |
| | | WO2016164731; | | WO2016164731; |
| | | Identifier 704 in | | Identifier 743 in |
| | | WO2016164731; | | WO2016164731; |
| | | Identifier 705 in | | Identifier 744 in |
| | | WO2016164731; | | WO2016164731; |
| | | Identifier 706 in | | Identifier 745 in |
| | | WO2016164731; | | WO2016164731; |
| | | Identifier 707 in | | Identifier 746 in |
| | | WO2016164731; | | WO2016164731; |
| | | Identifier 708 in | | Identifier 747 in |
| | | WO2016164731; | | WO2016164731; |
| | | Identifier 709 in | | Identifier 748 in |
| | | WO2016164731; | | WO2016164731; |
| | | Identifier 711 in | | Identifier 749 in |
| | | WO2016164731; | | WO2016164731; |
| | | Identifier 712 in | | Identifier 750 in |
| | | WO2016164731; | | WO2016164731; |
| | | Identifier 713 in | | Identifier 752 in |
| | | WO2016164731; | | WO2016164731; |
| | | Identifier 714 in | | Identifier 753 in |
| | | WO2016164731; | | WO2016164731; |
| | | Identifier 715 in | | Identifier 754 in |
| | | WO2016164731; | | WO2016164731; |
| | | Identifier 716 in | | Identifier 755 in |
| | | WO2016164731; | | WO2016164731; |
| | | Identifier 717 in | | Identifier 756 in |
| | | WO2016164731; | | WO2016164731; |
| | | Identifier 718 in | | Identifier 757 in |
| | | WO2016164731; | | WO2016164731; |
| | | Identifier 719 in | | Identifier 758 in |
| | | WO2016164731; | | WO2016164731; |
| | | Identifier 720 in | | Identifier 759 in |
| | | WO2016164731; | | WO2016164731; |
| | | Identifier 721 in | | Identifier 760 in |
| | | WO2016164731; | | WO2016164731; |
| | | Identifier 722 in | | Identifier 761 in |
| | | WO2016164731; | | WO2016164731; |
| | | Identifier 723 in | | Identifier 762 in |
| | | WO2016164731; | | WO2016164731; |
| | | Identifier 724 in | | Identifier 763 in |
| | | WO2016164731; | | WO2016164731; |
| | | Identifier 725 in | | Identifier 764 in |
| | | WO2016164731; | | WO2016164731; |
| | | Identifier 726 in | | Identifier 765 in |
| | | WO2016164731; | | WO2016164731; |
| | | Identifier 727 in | | Identifier 766 in |
| | | WO2016164731; | | WO2016164731; |
| | | Identifier 728 in | | Identifier 767 in |
| | | WO2016164731; | | WO2016164731; |
| | | Identifier 729 in | | Identifier 768 in |
| | | WO2016164731; | | WO2016164731; |
| | | Identifier 730 in | | Identifier 769 in |
| | | WO2016164731; | | WO2016164731; |
| | | Identifier 731 in | | Identifier 770 in |
| | | WO2016164731; | | WO2016164731; |
| | | Identifier 732 in | | Identifier 771 in |
| | | WO2016164731; | | WO2016164731; |
| | | Identifier 733 in | | Identifier 772 in |
| | | WO2016164731; | | WO2016164731; |
| | | Identifier 734 in | | Identifier 773 in |
| | | WO2016164731; | | WO2016164731; |
| | | Identifier 735 in | | Identifier 774 in |
| | | WO2016164731; | | WO2016164731; |
| | | Identifier 736 in | | Identifier 775 in |
| | | WO2016164731; | | WO2016164731; |
| | | Identifier 737 in | | Identifier 776 in |
| | | WO2016164731; | | WO2016164731; |
| | | Identifier 738 in | | Identifier 777 in |
| | | WO2016164731 | | WO2016164731; |
| | | | | Identifier 124 in |
| | | | | WO2016122701 |
| CTLA4 | VH | Identifier 3, 31, 32, 33, | VL | Identifier 36, 37, 38, |
| | | 34, 35, 41, 42, 43, 44, | | 39, 40, 46, 47, 48, 49, |
| | | 47, 7 in | | 50, 8, 4 in |
| | | US20140105914; | | US20140105914; |
| | | Identifier 4 in | | Identifier 2 in |
| | | US8697845; | | US8697845; |
| | | Identifier 19 in | | Identifier 20 in |
| | | US20150283234; | | US20150283234; |
| | | Identifier 17 in | | Identifier 18 in |
| | | WO2014066532 | | WO2014066532 |
| Integrin | VH | Identifier 3, 4, 5 in | VL | Identifier 10, 11, 8, 9 |
| | | US20140161794 | | in US20140161794 |
| IL13 | VH | Identifier 302 in | VL | Identifier 303 in |
| | | US20160168242 | | US20160168242 |
| Human | VH | Identifier 31 in | VL | Identifier 32 in |
| collagen VII | | WO2016112870 | | WO2016112870 |
| NKG2A | VH | Identifier 32 in | VL | Identifier 33 in |
| | | WO2016126213A1; | | WO2016126213A1; |
| | | Identifier 2, 3, 4, 5, 6 | | Identifier 7 in |
| | | in WO2016041947 | | WO2016041947 |
| Campath 1 | VH | Identifier 34 in | VL | Identifier 31, 33 in |
| | | US20160333114Al | | US20160333114A1 |
| KIR2DL 1 | VH | Identifier 36 in | VL | Identifier 37 in |
| | | WO2016126213A1 | | WO2016126213A1 |
| KIR2DL 2/3 | VH | Identifier 36 in | VL | Identifier 37 in |
| | | WO2016126213A1 | | WO2016126213A1 |
| VISTA | VH | Identifier 37, 38, 39, | VL | Identifier 41, 42, 43, |
| | | 40 in | | 44, 45 in |
| | | WO2015097536 | | WO2015097536 |
| CD46 | VH | Identifier 39, 47, 59, | VL | Identifier 41, 61, 21, |
| | | 15, 19, 23, 27, 31, 35, | | 25, 29, 33, 37, 45, 49, |
| | | 43, 51, 55, 63, 67, 71, | | 53, 57, 65, 69, 73, 77, |
| | | 75, 79, 83, 69, 71, 83 | | 81, 85, 17, 73, 77 |
| | | in WO2012031273; | | in WO2012031273; |
| | | Identifier 1, 10, 11, | | Identifier 23, 24, 25, |
| | | 12, 13, 14, 15, 16, 17, | | 26, 27, 28, 29, 30, 31, |
| | | 3, 5, 6, 7, 9, 18, 19, | | 32, 33, 34, 35, 36, 37, |
| | | 20, 21 in | | 38, 39, 40, 41, 42 in |
| | | WO2016040683 | | WO2016040683 |
| RHAMM | VH | Identifier 4 in | — | — |
| | | US20020127227A1 | | |
| VEGF | VH | Identifier 4, 8, in | VL | Identifier 2, 6 in |
| | | WO2000034337; | | WO2000034337; |
| | | Identifier 12, 20, 4, | | Identifier 9 in |
| | | 44 in | | US20030175276A1; |
| | | WO2006012688A1; | | Identifier 11, 19, 27, |
| | | Identifier 7 in | | 28, 3, 43 in |
| | | US20030175276A1; | | WO2006012688A1; |
| | | Identifier 152, 153, | | Identifier 160, 161, |
| | | 154, 155, 156, 157, | | 162, 163, 164, 165, |
| | | 158, 159 in | | 166, 167 in |
| | | US20160090427 | | US20160090427 |
| Endoglin | VH | Identifier 41, 42, 43, | VL | Identifier 103, 88, 89, |
| | | 71, 73, 75, 88, 89, 90, | | 90, 91, 92, 93, 94, 95, |
| | | 91, 92 in | | 96, 97, 102, 100 in |
| | | US20160009811 | | WO2011041441; |
| | | | | Identifier 100, 102, |
| | | | | 103, 3, 4, 5, 70, 72, |
| | | | | 74, 93, 94, 95, 96, |
| | | | | 97 in |
| | | | | US20160009811 |
| PRP | VH | Identifier 42 in | VL | Identifier 39 ,41 in |
| | | US20160333114A1 | | US20160333114A1 |
| Lysyloxidase- | VH | Identifier 42, 44 in | VL | Identifier 43, 45 in |
| like 2 | | WO2011097513 | | WO2011097513 |
| PSMA | VH | Identifier 43 in | VL | Identifier 44 in |
| | | WO2016097231 | | WO2016097231; |
| | | | | Identifier 44 in |
| | | | | WO2016097231 |
| PDL2 | VH | Identifier 43, 44, 56, | VL | Identifier 47, 48, 49, |
| | | 46 in | | 50, 51 in |
| | | US20130291136 | | US20130291136 |
| Daclizumab | VH | Identifier 44, 46 in | VL | Identifier 43, 45 in |
| | | US20160333114A1 | | US20160333114A1 |
| CD20 | VH | Identifier 45 in | VL | Identifier 46 in |
| | | WO201697231; | | WO2016097231; |
| | | Identifier 11, 13, 14, | | Identifier 10, 12, 8 |
| | | 15, 16, 17, 18, 19, 20, | | in WO2017004091; |
| | | 21, 22, 23, 24, 25, 26, | | Identifier 51 in |
| | | 27, 28, 29, 30, 31, 32, | | US20160333114Al |
| | | 33, 7, 9 in | | |
| | | WO2017004091; | | |
| | | Identifier 26 in | | |
| | | US20170000900; | | |
| | | Identifier 54 in | | |
| | | US20160333114Al; | | |
| | | Identifier 25 in | | |
| | | US20170000900; | | |
| | | Identifier 24 in | | |
| | | US20170000900; | | |
| | | Identifier 23 in | | |
| | | US20170000900 | | |
| Klon43 | VH | Identifier 47 in | VL | Identifier 48 in |
| | | WO2016097231 | | WO2016097231 |
| MUC1 | VH | Identifier 5 in | VL | Identifier 7 in |
| | | US20160130357; | | US20160130357; |
| | | Identifier 2, 14 in | | Identifier 16, 7 in |
| | | WO2013023162; | | WO2013023162; |
| | | Identifier 15, 19, 23, | | Identifier 17, 21, 25, |
| | | 60, 64, 68 in | | 62, 66, 70 in |
| | | WO2015116753 | | WO2015116753 |
| CD4i | VH | Identifier 5 in | VL | Identifier 4 in |
| | | US20160194375Al | | US20160194375A1 |
| BAT1 | VH | Identifier 5, 6, 7, 8, 9 | VL | Identifier 1, 2, 3, 4 |
| | | in WO2013014668 | | in WO2013014668 |
| PRAME | VH | Identifier 50, 52, 54, | VL | Identifier 49, 51, 53, |
| | | 56, 58, 60, 62 in | | 55, 57, 59, 61 in |
| | | WO2016191246A2 | | WO2016191246A2 |
| CD19 | VH | Identifier 53, 55 in | VL | Identifier 27, 31 in |
| | | WO2016120216 | | WO2016168773 A3; |
| | | | | Identifier 49 in |
| | | | | WO2016187349A1; |
| | | | | Identifier 11 in |
| | | | | WO2016134284; |
| | | | | Identifier 194 in |
| | | | | US20140134142Al; |
| | | | | Identifier 54, 56 in |
| | | | | WO2016120216; |
| | | | | Identifier 13, 14, 15, |
| | | | | 16, 17, 186, 187, 188, |
| | | | | 189, 192, 196, 197, |
| | | | | 198, 199, 200, 201, |
| | | | | 202, 203, 204, 205, |
| | | | | 64, 66, 67, 68, 69, |
| | | | | 70, 71, 91 in |
| | | | | US20160152723; |
| | | | | Identifier 22 in |
| | | | | US20160039942; |
| | | | | 3361 Identifier 63 in |
| | | | | WO2016097231; |
| | | | | Identifier 3 in |
| | | | | US20160145337Al; |
| | | | | Identifier 112 in |
| | | | | US20160333114Al; |
| | | | | Identifier 114 in |
| | | | | US20160333114A1; |
| | | | | Identifier 13, 6 |
| | | | | US20160319020 |
| Notum | VH | Identifier 56, 331 in | VL | Identifier 332, 58 in |
| | | WO2012027723 | | WO2012027723 |
| NOTCH 1 | VH | Identifier 58 in | VL | Identifier 16, 20 in |
| | | US20160333114A1; | | WO2013074596; |
| | | Identifier 12 in | | Identifier 55, 57 in |
| | | WO2013074596 | | US20160333114Al |
| Osteonectin | VH | Identifier 58 in | VL | Identifier 59 in |
| | | WO2016112870 | | WO2016112870 |
| CD74 | VH | Identifier 6 in | VL | Identifier 25, 29, 31, |
| | | US20100284906Al; | | 35 in |
| | | Identifier 10, 11, 9 in | | US20130171064; |
| | | US20040115193Al; | | Identifier 12, 13, 14, |
| | | Identifier 23, 27, 30, | | 11, 4 in |
| | | 33 in | | US20040115193Al |
| | | US20130171064 | | |
| CD33 | vH | Identifier 65, 67, 69, | VL | Identifier 12, 14, 16, |
| | | 71, 77, 79, 81, 83, | | 18 in |
| | | 84 in | | WO2015150526A2; |
| | | WO2016120216; | | Identifier 66, 68, 70, |
| | | Identifier 11, 13, 15; | | 72, 78, 80, 82 in |
| | | 17 in | | WO2016120216; |
| | | WO2015150526A2; | | Identifier 66, 67, 68, |
| | | Identifier 57, 58, 59, | | 69, 70, 71, 72, 73, 74 |
| | | 60, 61, 62, 63, 64, 65 | | in WO2016014576 |
| | | in WO2016014576 | | |
| Rituximab | VH | Identifier 66 in | VL | Identifier 63, 65 in |
| | | US20160333114Al | | US20160333114Al |
| GAH | VH | Identifier 7 in | VL | Identifier 8 in |
| | | US20060057147A1 | | US20060057147A1 |
| Glyco epitope | VH | Identifier 7 in | VL | Identifier 10 in |
| and ErbBB I | | WO2012007167A1 | | WO2012007167A1 |
| Specific | | | | |
| CD3s | VH | Identifier 7 in | VL | Identifier 8 in |
| | | WO2014144722A2 | | WO2014144722A2 |
| IGFR1 | VH | Identifier 7 in | VL | Identifier 8 in |
| | | WO2015073575A2 | | WO2015073575A2 |
| IL13Ra2 | VH | Identifier 7, 8 in | — | — |
| | | WO2016123143 | | |
| N Glycan | VH | Identifier 7, 9 in | VL | Identifier 6, 8 in |
| | | US20160194375A1 | | US20160194375A1 N |
| Upar | VH | Identifier 72 in | VL | Identifier 71, 73 in |
| | | US20160333114Al | | US20160333114Al |
| CXCR4 | VH | Identifier 72, 73, 74, | VL | Identifier 76, 77, 78, |
| | | 75, 84 in | | 79, 80, 81, 82, 87, 88, |
| | | US20110020218 | | 90, 91, 92, 93 in |
| | | | | US20110020218 |
| Tie | VH | Identifier 723 in | VL | Identifier 724 in |
| | | US20060057138Al | | US20060057138Al |
| CEA | VH | Identifier 8 in | VL | Identifier 10, 38, 39, |
| | | US8287865 | | 7, 9 in US8287865 |
| Herl/her3 | VH | Identifier 8 of | VL | Identifier 4 of |
| | | WO2016073629 | | WO2016073629 |
| CD70 | VH | Identifier 81, 85, 89 in | VL | Identifier 83, 87, 91 |
| | | WO2015121454 | | in WO2015121454; |
| | | | | Identifier 83 in |
| | | | | WO2015121454; |
| | | | | Identifier 87 in |
| | | | | WO2015121454; |
| | | | | Identifier 91 in |
| | | | | WO2015121454 |
| leuko- | VH | Identifier 9 in | VL | Identifier 24 in |
| cytegenA | | WO2010065962 A2 | | WO2010065962 A2 |

In some embodiments, the antigen-binding moiety may comprise an scFv derived from an antibody or antibody fragment that binds to an antigen target such as those described in the cited publications, the contents of each publication are incorporated herein by reference in their entirety for all purposes (Table 2).

TABLE 2
Exemplary antigen-binding moieties comprising an scFv derived from
an antibody or antibody fragment that binds to an antigen target

| Antigen Target | Examples of Source |
|---|---|
| CEA | Identifier 1 in US20160303166A1; Identifier 22 in US20140242701A1 |
| GPC3 | Identifier 1 in WO2016049459; Identifier 12 in US20160208015A1 |
| CS1 | Identifier 1 of WO2016090369; Identifier 17 in WO2014179759A1 |
| VEGFR2 | Identifier 1, 2 in US20120213783 |
| TSLPR | Identifier 1, 2 in US20160311910A1; Identifier 1, 2 in WO2015084513 |
| B7H4 | Identifier 1, 2, 3, 4 in WO2013067492; Identifier 1 in U.S. Pat. No. 9,422,351B2 |
| CD276 | Identifier 10, 19, 28 in US20160053017 |
| GPRC5D | Identifier 100, 101, 102, 103, 104, 105, 106, 107, 108, 109, 110, 111, |
| | 112, 113, 114, 115, 116, 117, 118, 119, 120, 121, 122, 123, 301, 313, |
| | 325, 337, 349, 361, 373, 385 in WO2016090312 |
| WT1/HLA | Identifier 108, 113, 18, 36, 54, 72, 90 in WO2015070061 |
| bispecific | |
| CMet | Identifier 11, 12, 13, 14, 15, 16, 17, 18, 19, 2, 21, 22, 23, 25, 26, 27, 28, 3, |
| | 30, 31, 33, 34, 35, 36, 37, 38, 39, 4; 40, 41, 42, 43, 44, 48, 49, 5, 50, 51, 52, |
| | 53, 54, 55, 56, 57, 58, 6, 60, 7, 9, 29 in US20040166544; Identifier 26, 27, |
| | 28, 29, 30, 32 in US20150299326; Identifier 32 in US20130034559 |
| FcRL | Identifier 11, 15, 19, 23, 27, 31, 35, 39, 3, 43, 7, 594, 596, 598, 600, 602, |
| | 604, 606, 608, 610, 612, 614, 616, 618, 620, 622, 624, 626, 628, 630, 632, |
| | 634, 636, 638, 640, 642, 644, 646, 648, 652, 654, 656; 658, 660, 662, 664, |
| | 666, 668, 670, 672, 674, 676, 680, 682, 684, 686, 688, 690, 692, 694, 696, |
| | 700, 702, 704, 706, 708, 710, 712, 714, 716, 718, 720, 722, 724, 726, 728, |
| | 730, 732, 734, 736, 738, 740, 742, 744, 746, 748, 750, 752, 754, 756, 758, |
| | 760, 762, 764, 766, 768, 770, 772, 774, 776, 778, 780, 782, 784, 786, 788, |
| | 790, 792, 794, 796, 798, 800, 802, 804, 806, 808, 810, 812, 814, 816, 818, |
| | 820, 822, 824, 826, 828, 830, 832, 834, 836, 838, 840, 842, 844; 846, 848, |
| | 850, 852, 854, 856, 858, 860, 862, 864, 866, 868, 870, 874, 876, 878, 880, |
| | 882, 884, 886, 888, 890, 892, 894, 896, 650, 678 in WO2016090337 |
| EGFR | Identifier 11, 38, 41, 44, 47, 50, 53, 56, 59, 62, 65, 68, 71, 74, 77, 80, |
| | 83, 88, 91, 94 in WO2014130657 |
| Claudin | Identifier 11, 5, 7, 9 in WO2016073649A1; Identifier 17 in |
| | WO2014179759A1 |
| PSMA diabody | Identifier 12, 13, 14, 15 in WO2011069019 |
| TRBC1 | Identifier 13, 14, 15, 16, 17, 18, 19, 20, 21, 22, 3 in WO2015132598 |
| CD19/CD22 | Identifier 1303, 1307 in WO2016164731A2 |
| Bispecific | |
| CD46 | Identifier 1-42 in WO2016040683 |
| MUC1 | Identifier 15 in US20160130357 |
| MUC3 | Identifier 15 in US20160130357 |
| Folate receptor | Identifier 15 in US20170002072A1 |
| PDK1 | Identifier 15 in WO2016090365 |
| Folate receptor | Identifier 15, 23 in WO2012099973 |
| alpha | |
| BCMA | Identifier 152, 158, 176, 185, 188, 200, 212, 218, 224, 284, 290, 296, |
| | 302, 314, 326, 344, 129, 130, 131, 132, 133, 134, 135, 136, 138, 139, |
| | 140, 141, 142, 143, 144, 145, 146, 147, 148, 149, 263, 264, 265, 266, |
| | 271, 273, 39, 40, 41, 42, 43, 44, 45, 46, 47, 48, 49, 50, 51, 52, 53, 64, |
| | 129, 130, 131, 132, 133, 134, 135, 136, 137, 138, 139, 140, 141, 142, |
| | 143, 144, 145, 146, 147, 148, 149, 263, 264, 265, 266, 39, 40, 41, 42, |
| | 43, 44, 45, 46, 47, 48, 49, 50, 51, 52, 53 in WO2016014565; Identifier |
| | 214, 215, 216, 217, 218, 219, 220, 221, 222, 223, 224, 225, 226, 227, |
| | 228, 229, 230, 231, 232, 233, 234, 235, 236, 237, 238, 239, 240, 241, |
| | 242, 243, 244, 245, 246, 247, 248, 249, 251 in US20160311907A1 |
| CD123 | Identifier 157, 158, 159, 160, 184, 185, 186, 187, 188, 189, 190, 191, |
| | 192, 193, 194, 195, 196, 197, 198, 199, 200, 201, 202, 203, 204, 205, |
| | 206, 207, 208, 209, 210, 211, 212, 213, 214, 215, 478, 480, 483, 485 in |
| | WO2016028896; Identifier 36 in WO2015092024A2; Identifier 57 in |
| | WO2016115482A1; Identifier 36 in EP3083691A2; Identifier 157 in |
| | US20160311907A1 |
| CD124 | Identifier 158 in US20160311907A1 |
| CD125 | Identifier 159 in US20160311907A1 |
| CD5 | Identifier 16 in WO2016138491 |
| CD126 | Identifier 160 in US20160311907A1 |
| CD127 | Identifier 161 in US20160311907A1 |
| CD128 | Identifier 162 in US20160311907A1 |
| CD129 | Identifier 163 in US20160311907A1 |
| CD130 | Identifier 164 in US20160311907A1 |
| Claudin6 | Identifier 164 in WO2016115482A1 |
| CD131 | Identifier 165 in US20160311907A1 |
| Claudin7 | Identifier 165 in WO2016115482A1 |
| GCN4 | Identifier 165, 166, 167, 168, 169, 170 in WO2016168773 A3 |
| Claudin8 | Identifier 166 in WO2016115482A1 |
| VEGF | Identifier 168, 169, 170, 171, 172, 173, 174, 175 in US20160090427; |
| | Identifier 498, 500, 502, 504, 506, 508 in US20110177074A1 |
| MUC2 | Identifier 17 in US20160130357 |
| MUC4 | Identifier 17 in US20160130357 |
| CD44 | Identifier 17 in WO2016042461A1 |
| IL4 | Identifier 17, 16 in WO2009121847 |
| ALK | Identifier 17, 18, 19, 20, 21, 22, 23 in WO2015069922; Identifier 17, 18, 19, |
| | 20, 21, 22, 23, 24 in US20160280798A1; Identifier 24 in WO23015069922 |
| ESKAVT | Identifier 173 in WO2016115482A1 |
| GD2 | Identifier 19, 20, 21, in WO2016134284; Identifier 8 in WO2015132604 |
| PSMA | Identifier 19, 21, 30, 31, 34, 35 in WO2012145714 |
| TOSO | Identifier 2 in EP3098237A1 |
| CSPG4 | Identifier 2 in WO2015080981; Identifier 2 in EP3074025A1 |
| CLDN6 | Identifier 2 in WO2016150400 |
| Integrin bivalent | Identifier 2, 1 in WO2009070753 |
| CD30 | Identifier 20 in WO2016116035A1; Identifier 2 in US20160200824A1 |
| NYBR1 | Identifier 21 in US20160333422A1; Identifier 21, 18, 19 in WO2015112830 |
| CD37 | Identifier 21, 22 in US20170000900 |
| PDL1 nanobody | Identifier 22, 23, 24, 25, 26, 27 in US20110129458 |
| Radiation inducible | Identifier 22, 24 in WO2005042780A1 |
| neoantigen | |
| E7MC | Identifier 223, 224, 225, 226, 227, 228, 229, 230, 231, 232 in |
| | WO2016182957A1 |
| TRBC2 | Identifier 23, 24, 25, 26, 27, 28, 29, 30, 31, 32 in WO2015132598 |
| GPC4 | Identifier 24 in WO2016049459 |
| ERBB2 | Identifier 26, 27 in US20110059076A1; Identifier 1, 2 in U.S. Pat. No. 7,244,826 |
| CD33 | Identifier 262, 263, 264, 265, 266, 267, 268, 39, 40, 41, 42, 43, 44, 45, |
| | 46, 47 in WO2016014576; Identifier 37 in WO2015092024 A2; |
| | Identifier 37 in EP3083691A2; Identifier 153, 154, 155, 156, 157, 158, |
| | 159, 160, 161, 162, 163 in WO2016115482A1 |
| PDL2 nanobody | Identifier 28, 29, 30, 31, 32, 33 in US20110129458 |
| 6462 | |
| O-acetylated GD2 | Identifier 29, 31 in US20150140023 |
| ganglioside | |
| OX40 | Identifier 33 in US20150190506 |
| CD79b | Identifier 33 in US20160208021 |
| Human CD79b | Identifier 33 in WO2016112870 |
| CD33/CD3s | Identifier 33, 34, 84 in WO2014144722A2 |
| bispecifc | |
| RORI | Identifier 34 in EP3083691A2; Identifier 249, 250 251, 252, 253, 254, |
| | 255, 256, 257, 258, 259, 260, 261, 262, 263, 264, 265, 266, 267, 268 in |
| | US20160208018A1; Identifier 57 in EP3083671A1; Identifier 1, 2 in |
| | US20160304619A1; Identifier 34 in WO2015092024A2 |
| Human collagen | Identifier 34 in WO2016112870 |
| VII | |
| Trastuzumab | Identifier 35 in US20160208021; Identifier 35 in WO2016112870 |
| Rituximab | Identifier 36 in US20160208021; Identifier 36 in WO2016112870 |
| CD138 | Identifier 36 in WO2016130598A1 |
| Centuxiamb | Identifier 37 in WO2016112870; Identifier 37 in US20160208021 |
| Nivolumab | Identifier 38 in US20160208021; Identifier 38 in WO2016112870 |
| Ipilimumab | Identifier 39 in US20160208021; Identifier 39 in WO2016112870 |
| PD1 | Identifier 39, 40, 41, 42, 43, 44, 45, 46, 47, 48, 49, 50, 51, 52, 53, 54, |
| | 55, 56, 57, 58, 59, 60, 61 in US20160311917A1 |
| CLL1 | Identifier 39, 40, 41, 42, 43, 44, 45, 46, 48, 49, 50, 51 in WO2016014535; |
| | Identifier 200, 201, 202, 203, 204, 205, 206, 207, 208, 209, 210, 211, |
| | 212, 213 in US20160311907A1 |
| CLDN7 | Identifier 4 in WO2016150400 |
| Ranibizuman | Identifier 40 in US20160208021; Identifier 40 in WO2016112870 |
| Adalimunab | Identifier 41 in US20160208021; Identifier 41 in WO2016112870 |
| Teplizumab | Identifier 42 in US20160208021 |
| (mutated) | |
| Teplizumab | Identifier 42 in WO2016112870 |
| CD3 | Identifier 46, 47 in WO2015153912A1 |
| EGFR VIII | Identifier 5 in US20140037628; Identifier 174 in US20160311907A1; |
| | Identifier 38 in U.S. Pat. No. 9,394,368B2; Identifier 5 in US20160200819A1 |
| CD22 | Identifier 5, 6 in WO2013059593; Identifier 9 in US20150299317; |
| | Identifier 131, 132, 133, 134, 135, 136, 137, 138, 139, 140, 203, 209, |
| | 215, 221, 227, 232, 238, 244, 250, 256, 262, 268, 274, 280, 286, 292, |
| | 298, 304, 310, 316, 322, 328, 334, 340, 346, 353, 358, 364, 370, 376, |
| | 383, 388, 394, 400, 406, 412, 418, 423 in WO2016164731A2 |
| CD19 | Identifier 53, 54, 37 in EP3083671A1; Identifier 1, 10, 11, 12, 2 in |
| | WO2015157252; Identifier 10, 2, 206, 207, 208, 209, 210, 211, 213, 214, |
| | 215, 216, 217, 218, 219, 220, 221, 222, 223, 224, 225, 4, 45, 47, 49, 51, |
| | 53, 55, 57, 51, 53, 55, 57, 59, 6, 8, 87 in WO2016033570; Identifier 3, 4, 5, |
| | 59, 6, 7, 8, 9 in WO2015157252; 5754 Identifier 5 in WO2015155341A1; |
| | Identifier 7 in WO2014184143; Identifier 9 in WO2016139487; Identifier |
| | 10, 2, 206, 207, 208, 209, 210, 211, 212, 213, 214, 215, 216, 217, 218, 219, |
| | 220, 221, 222, 223, 224, 225, 4, 45, 47, 49, 51, 53, 55, 57, 59, 6, 8, 87, 89 in |
| | US20160152723; Identifier 32, 35, 38 in EP3083691A2; Identifier 174 in |
| | WO2016115482A1; Identifier 20 in WO2012079000; Identifier 32, 33, 35, |
| | 38 in WO2015092024A2; Identifier 39, 40, 41, 42, 43, 44, 45, 46, 47, 48, |
| | 49, 50, 51 in WO2016109410; Identifier 5, 6 in WO2015155341A1; |
| | Identifier 7, 9 in US20160145337A1; Identifier 20 in U.S. Pat. No. 9,499,629B2; |
| | Identifier 73 in WO2016164580; Identifier 10, 2, 206, 207, 209, 210, |
| | 216, 218, 219, 220, 221, 222, 223, 224, 225, 4, 45, 47, 49, 51, 53, 55, 57, |
| | 59, 6, 8, 87, 89 in US20160152723; Identifier 5 in WO2016055551 |
| END 0180 | Identifier 6 in WO2013098813 |
| CLDN8 | Identifier 6 in WO2016150400 |
| PRAME | Identifier 63, 64, 65, 66, 67, 68, 69 in WO2016191246A2 |
| CD20 | Identifier 691 in WO2016164731A100; Identifier 692 in |
| | WO2016164731A101; Identifier 693 in WO2016164731A102; Identifier |
| | 694 in WO2016164731A103; Identifier 695 in WO2016164731A104; |
| | Identifier 696 in WO2016164731A105; Identifier 175 in WO2016115482A1 |
| Mesothelin | Identifier 7 WO2015188141; Identifier NO 47, 46, 57, 48, 49, 50, 51, |
| | 53, 54, 55, 56, 58, 59, 62, 64, 65, 66, 67, 68, 69, 70, 52, 60, 61, 63 in |
| | WO2016090034; Identifier 10, 11, 12 in WO2013142034; Identifier 11 |
| | in WO2013063419 |
| CCR4 | Identifier 7, 9 in WO2015191997 |
| Mec/CD3s | Identifier 78 in WO2014144722A2 |
| bispecific | |
| Activated alpha | Identifier 8, 2, 4 in US20090117096A1 |
| v beta 3 | |
| RAS | Identifier 81 in WO2016154047 |
| CXCR4 | Identifier 83, 85, 86, 89 in US20110020218 |
| HER2/CD3 | Identifier 9 in WO2014144722 A2 |
| FOLR1/CD3s | Identifier 90 in WO2014144722A2 |
| bispecific | |
| B7H3 | Identifier 99, 100, 101, 102, 103, 104, 102, 17, 18, 19, 20, 21, 22, 23, 24, 25, |
| | 26, 27, 87, 88, 89, 90, 91, 92, 94, 95, 96, 97, 98 in WO2016033225 |

In some embodiments, the antigen-binding moiety may comprise an antigen-binding moiety derived from a CAR that binds to an antigen target, such as those described in the cited publications, the contents of each publication are incorporated herein by reference in their entirety for all purposes (Table 3).

TABLE 3
Exemplary antigen-binding moieties comprising an antigen-binding
moiety derived from a CAR that binds to an antigen target

| Antigen Target | Examples of Source |
|---|---|
| CAR or gate (DC19 | Identifier 1 in US20160296562 |
| or DC33) | |
| CD19 or CD33 | Identifier 1 in WO2015075468 which recognizes CD19 OR CD33 |
| CD19/IL13 bispecific | Identifier 10 in US20160340649A1 |
| CAT19, campana | Identifier 10 in WO2016139487 |
| architecture | |
| CD2 | Identifier 10, 11 in WO2016138491 |
| CD70 | Identifier 100, 93, 94, 96, 101, 95, 97, 98 in WO2015121454 |
| VNAR | Identifier 105, 106, 107, 108, 109, 110 in US20160333094A1 |
| FcRL5 | Identifier 11 in US20170008963A1 |
| CAT19 CAR with | Identifier 11 in WO2016139487 |
| OX40 zeta endodomain | |
| Folate receptor | Identifier 12 in US20170008963A1 |
| GD2 | Identifier 12 in U.S. Pat. No. 9,446,105B2; Identifier 273, 274 in |
| | WO2016168773A3; Identifier 26, 27, 28, 29, 30, 31, 32, 33, 34, |
| | 35, 36, 37 in WO2015132604; WO2016134284 (no Identifier) |
| CD19 | Identifier 12 in U.S. Pat. No. 9,499,629B2; Identifier 24 in US20160333108A1; |
| | Identifier 25, 29 in US20160333108A1; Identifier 27 in |
| | US20160333108A1; Identifier 1 in EP2997134A4; Identifier 19, 20 |
| | in EP3071687A1; Identifier 181 in WO2016168773A3; Identifier 2 |
| | in WO2015157399A9; Identifier 56, 62 in WO2016174409A1; |
| | Identifier 145, 293, 294, 295, 296, 297, 298 in WO2016179319A1; |
| | Identifier 73 in WO2013176915A1; Identifier 73 in |
| | WO2013176916A1; Identifier 73 in US20130315884A1; Identifier |
| | 73 in US20140134142A1; Identifier 73 in US20150017136A1; |
| | Identifier 73 in US20150203817A1; Identifier 73 in |
| | US20160120905A1; Identifier 73 in US20160120906A1; Identifier |
| | 8, 5 in WO2015124715; Identifier 73 in WO2014184744; Identifier |
| | 73 in WO2014184741; Identifier 14, 15 in US20160145337A1; |
| | Identifier 14, 15 in WO2014184143; Identifier 15, 16 in |
| | WO2015075175; Identifier 16 in US20160145337A1; Identifier 16 in |
| | WO2014184143; Identifier 12 in WO2012079000; Identifier 31, 32, 33, |
| | 34, 35, 36, 37, 38, 39, 40, 41, 42, 58 in WO2016164580; Identifier 14, |
| | 15 in US20160296563A1; Identifier 31, 32, 33, 34, 35, 36, 37, 38, 39, |
| | 40, 41, 42 in WO2015157252; Identifier 14, 15 in WO2016139487; |
| | Identifier 53, 54, 55, 56, 57, 58 in US20160250258A1; Identifier 1, 2, |
| | 3, 4, 5, 6, 7, 8, 9, 10, 11, 12, 13 in WO2015187528; Identifier 31, 32, |
| | 33, 34, 35, 36, 37, 38, 39, 40, 41, 42, 58 in WO2015157252; Identifier |
| | 31, 32, 33, 34, 35, 36, 37, 38, 39, 40, 41, 42 in WO2014153270; |
| | WO2016134284 (no Identifier); Identifier 13 in WO2016139487 |
| CD3 | Identifier 12 in WO2016138491 |
| CAT19 CAR with | Identifier 12 in WO2016139487 |
| CD28 zeta endodomain | |
| FRa | Identifier 13, 14 in US20120213783 |
| CD4 | Identifier 13, 14 in WO2016138491 |
| FR beta | Identifier 13, 22 in U.S. Pat. No. 9,402,865B2; Identifier 2, 4, 6 in U.S. Pat. No. 9,446,105B2 |
| CD19/CD20 | Identifier 1308 in WO2016164731A2; Identifier 2, 8, 11 in |
| bispecific | U.S. Pat. No. 9,447,194B2 |
| CLL1 | Identifier 148 in WO2016179319A1; Identifier 35, 36, 37, 38, 39, |
| | 40, 41, 42, 43, 44, 45, 46, 47, 48, 49, 50, 51, 52, 53, 54, 55, 56, 57, |
| | 58, 59, 60, 61, 62, 63, 64, 65, 66, 67, 68, 69, 70, 71, 72, 73, 74, 75, |
| | 76, 77, 78, 79, 80, 81, 82, 83, 84, 85, 86, 87, 88, 89, 90, 91, 92, 93, |
| | 94, 95, 96, 97, 98, 99, 100, 101, 102, 103, 104, 105, 106, 107, 108, |
| | 109, 110, 111, 112 in WO2016120218; Identifier 91, 92, 93, 94, |
| | 95, 96, 97, 98, 99, 100, 101, 102, 103, 197 in WO2016014535 |
| CD5 | Identifier 15, 13 in WO2016138491 |
| EGFR vIII | Identifier 15, 16, 17, 18, 24, 25, 26, 27 in WO2016016341; |
| | Identifier 5, 10, 12, 8, 31, 30, 3 in US20160311907A1; Identifier |
| | 10 in US20160200819A1; Identifier 43, 49, 55, 61, 67, 73, 79, 85, |
| | 90, 96 in U.S. Pat. No. 9,394,368B2; Identifier 49, 55, 61, 67, 73, 79, 85, 90, |
| | 2, 1 in US20170008963A1; Identifier 10, 11 in US20140037628 |
| CD7 | Identifier 17 in WO2016138491 |
| CD52 | Identifier 18 in WO2016138491 |
| BCMA | Identifier 180, 162, 168, 174, 144, 150, 186, 192, 198, 204, 210, 156, |
| | 216, 222, 228, 234, 240, 246, 252, 258, 264, 270, 276 330, 282, 300, |
| | 306, 336, 354, 288, 312, 294, 342, 324, 318, 348 in WO2016168595A1; |
| | Identifier 48, 49, 50, 51, 52, 53, 54, 55, 56, 57, 58, 59, 19, 20, 21, 22, |
| | 23, 24, 25, 26, 27, 28, 29, 30, 31, 32, 33, 34, 35, 36, 37, 38, 39, 40, 41, |
| | 42 in WO2015158671A1; Identifier 124, 114, 115, 116, 117, 118, 119, |
| | 120, 121, 122, 123, 125, 126, 127, 128, 234, 235, 236, 237, 238, 239, |
| | 240, 241, 242, 243, 244, 245, 246, 247, 248, 249, 250, 251, 252, 253, |
| | 254, 267, 268, 269, 270 in WO2016014565; Identifier 1, 2, 3, 4, 5, 20 |
| | in WO2015052538; Identifier 1, 2, 3, 4, 5, 6 in US20160237139A1; |
| | Identifier 9 in WO2016094304 A3; Identifier 4, 5, 6, 8, 9, 10, 11, 12 in |
| | WO2013154760; Identifier 15, 16, 17, 18, 19, 20, 21, 22, 23, 24, 25, |
| | 26, 27, 28, 29, 71, 73 in WO2016014789; Identifier 125, 126, 127, |
| | 128, 129, 130, 131, 132, 133, 134, 135, 136, 137, 138, 139, 140, 141, |
| | 145, 146, 147, 148, 149, 150 in WO2016120216; Identifier 102, 106, |
| | 107, 108, 109, 110, 111, 112, 129, 130, 131, 132, 133, 134, 135, 136, |
| | 113, 114, 115, 116, 117, 118, 101, 100, 137, 119, 120, 121, 122, 123, |
| | 124, 125, 126, 127, 128, 103, 104, 105, 213 in WO2016097231 |
| Tan | Identifier 19 in US20160311907A1 recognizes CD19 AND CD33 |
| | using a CD45 phosphatase; Identifier 5 in WO2016026742A1 |
| | recognizes CD19 AND CD33 using a CD148 phosphatase; Identifier 6 |
| | in WO2016026742A1 which recognizes CD19 AND NOT CD33 and is |
| | based on an ITIM containing endodomain from LAIR1; Identifier 3 in |
| | WO2015075468 which recognizes CD19 AND NOT CD33 and |
| | PTPN6 phosphatase; Identifier 2 in WO2015075468 which recognizes |
| | CD19 AND NOT CD33 and recruits a PTPN6/CD148 fusion protein |
| | to an ITIM containing endodomain |
| GD3 | Identifier 19 in WO2016185035A1; Identifier 20, 21, 22, 23, 24, |
| | 25, 26 in WO2016185035A1; WO2016134284 (no Identifier) |
| CAR and gate | Identifier 2 in US20160296562 |
| (CD19 and CD33) | |
| CD148 phosphatase | |
| CD30 | Identifier 20 in WO2016008973A1; Identifier 1 in WO2016116035A1; |
| | WO2016134284 (no Identifier); Identifier 2 in WO2016008973 |
| CD44 | Identifier 21, 22, 23, 24, 25, 26, 27, 28, 31, 32, 33, 34, 35 in |
| | WO2016042461A1 |
| ROR1 | Identifier 216, 217, 215 in WO2016097231; Identifier 79, 80, 81, 82, |
| | 83, 84, 85, 86, 87, 88, 89, 90, 91, 92, 93, 94, 95, 96, 103, 104, 105, 106, |
| | 107, 108, 109, 110, 111, 112, 113, 114, 115, 116, 117, 118, 119, 120, |
| | 127, 128, 129, 130, 131,132, 133, 134, 1335, 136, 137, 138, 97, 98, 99, |
| | 100, 101, 102, 121, 122, 123, 124, 125, 126 in WO2016016344A1; |
| | Identifier 386, 387, 388, 389, 390, 391, |
| FR | Identifier 22 in US20170002072A1 |
| CLDN6 | Identifier 22, 23, 24 in WO2016150400 |
| PD1 | Identifier 23 in WO2016014565; Identifier 26 in WO2015142675 |
| Her1/Her3 bispecific | Identifier 23, 24 in US20160215261A1 |
| HER2 | Identifier 25 in US20160215261A1; Identifier 9, 10 of |
| | WO2016073629; Identifier 17, 28, 98, 110 in US20160333114A1; |
| | Identifier 271, 272 in WO2016168773A3; Identifier 5 in |
| | WO2016168769A1 |
| CD20 | Identifier 25 in WO2015157399A9; Identifier 177, 181, 182, 183, |
| | 184, 185, 186, 187, 205, 206, 207, 208, 209, 210, 211, 188, 189, |
| | 190, 191, 192, 193, 176, 212, 194, 195, 196, 197, 198, 199, 200, |
| | 201, 202, 203, 178, 179, 180 in WO2016097231 |
| P5A | Identifier 26, 29, 60 in US20160333422A1 |
| GFR alpha | Identifier 27 in WO2016185035A1 |
| GPC3 | Identifier 28, 29 in WO2016185035A1; Identifier 3, 27, 10, 29, 14, 30, |
| | 31, 18, 33 in WO2016049459; Identifier 22 in US20160215261A1 |
| CD22/CD19 | Identifier 29, 30 in WO2016149578; Identifier 1304 in |
| bispecific | WO2016164731A2 |
| CAR and gate | Identifier 3 in US20160296562 |
| (CD19 or CD33) | |
| CD45 phosphatase | |
| EGFR | Identifier 3, 2in WO2014130657; Identifier 36, 37, 38, 39, 35 in |
| | US20140242701 A; Identifier 43, 96, 49, 55, 61, 67, 73, 79, 85, |
| | 90, 1 in WO2014130657 |
| IL13 | Identifier 30, 31, 32 in WO2016120217 |
| Acid/base leucine | Identifier 34, 35 in WO2016124930 |
| zipper | |
| P5AC1 | Identifier 343, 344, 345, 346 in US20160297884A1 |
| P5AC16 | Identifier 347, 396, 348 in US20160297884A1 |
| P6AP | Identifier 349, 350, 351 in US20160297884A1 |
| PC1C12 | Identifier 352, 353, 354 in US20160297884A1 |
| PD1 | Identifier 355, 356, 357 in US20160297884A1; Identifier 119 in |
| | WO2014153270; Identifier 121 in WO2014153270; Identifier 22, |
| | 24, 63, 64, 65, 66, 67, 68, 69, 70, 71, 72, 73, 74, 75, 76, 77, 78, 79, |
| | 80, 81, 82, 83, 84, 85, 86 in US20160311917A1; Identifier 26, 39 |
| | in WO2016172537A1; Identifier 40 in US20160311907A1; |
| | Identifier 121, 119 in WO2015157252; Identifier 24 in |
| | WO2016014565; Identifier 22 in WO2016014565 |
| COM22 | Identifier 358, 359, 360 in US20160297884A1 |
| DDD1/AD1-based | Identifier 36 in WO2016124930 |
| zip | |
| PCI | Identifier 361, 362, 363 in US20160297884A1 |
| P6DY | Identifier 364, 365, 366 in US20160297884A1 |
| DDD1/AD1 zip | Identifier 37 in WO2016124930 |
| CD22 | Identifier 380, 204, 260, 266, 272, 278, 284, 290, 296, 302, 308, |
| | 341, 213, 320, 326, 332, 338, 347, 350, 356, 362, 368, 374, 219, |
| | 386, 392, 398, 404, 410, 416, 421, 427, 225, 230, 1109, 236, 242, |
| | 248, 254 in WO2016164731A2; Identifier 15, 16, 17, 18, 19, 20, |
| | 32 in WO2013059593; Identifier 22, 23, 24 in US20150299317 |
| PSMA | Identifier 39 in WO2015142675; Identifier 28, 29 in |
| | US20160311907A1; Identifier 140, 144, 145, 146, 147, 148, 149, |
| | 150, 167, 168, 169, 170, 171, 172, 173, 174, 151, 152, 153, 154, |
| | 155, 156, 139, 138, 175, 157, 158, 159, 160, 161, 162, 163, 164, |
| | 165, 166, 141, 142, 143, 214 in WO2016097231 |
| CD276 | Identifier 39, 40, 41, 42, 43, 44, 45, 46, 47, 122, 123, 124, 125, |
| | 126, 127, 128, 129, 130 in US20160053017 |
| SNAP | Identifier 395 in WO2016187216A1 |
| SSEA4 | Identifier 396, 397 in WO2016187216A1 |
| CEA | Identifier 4 in WO2016008973A1; Identifier 29, 30 in |
| | US20140242701A |
| CAR and not gate | Identifier 4, 5 in US20160296562 |
| (CD19 and not CD33) | |
| IL13Ra2specific | Identifier 4, 5, 6 in WO2016089916A1; Identifier 47, 49 in |
| | WO2016123143; Identifier 51, 53, 55 in WO2016123143; |
| | Identifier 1, 31, 32, 33, 34, 35, 36, 37, 38, 39, 40, 41, 42, 43, 44, |
| | 45 in US20160340649A1 |
| TSLPR | Identifier 40, 41, 42 in WO2016034666A1; Identifier 39, 40, 41, |
| | 42, 43, 44, 45, 46 in WO2015084513; Identifier 39, 40, 41, 42, 43 |
| | in US20160311910A1 |
| CAR and gate | Identifier 41 in US20160296562 |
| (CD19 and GD2) | |
| CAR and gate | Identifier 43 in US20160296562 |
| (CD19 and CD5) | |
| ALK | Identifier 43, 44, 45, 46, 47, 48, 49, 50, 51, 52, 53, 54, 55, 56, 57, 58, |
| | 59, 60, 61, 62, 63, 64, 65, 66, 67, 68, 69, 70, 71, 72, 73, 74, 75, 76, 77, |
| | 78, 79, 80, 81, 82, 84, 85, 86, 87, 88, 89, 90 in WO2015069922 |
| VEGFR2 | Identifier 44, 45, 46 in US20160311910A1; Identifier 10, 11, 12 in |
| | US20120213783 |
| CAR and gate (CD19 | Identifier 45 in US20160296562 |
| and EGFR VIII) | |
| KMA | Identifier 46 in US20160340649A1 |
| Mesothelin | Identifier 47, 48 in US20160340649A1; Identifier 48 in |
| | US20160340649A1; Identifier 27 in WO2016172703A2; Identifier |
| | 18, 19, 20, 21, 22, 23 in WO2013142034; Identifier 3 in |
| | WO2013067492 |
| CAR and not gate 1 | Identifier 48 in US20160296562 |
| HIV Env | Identifier 48, 49 in WO2016168766A1; Identifier 4 in |
| | EP2997134A4; Identifier 7, 9, 47, 49 in WO2015077789 |
| CD33 | Identifier 48, 49, 50, 51, 52, 53, 54, 55, 83 in WO2016014576; |
| | Identifier 19, 20, 21, 22, 23, 24, 25, 26, 27, 28, 29, 30, 31, 32, 33, 34, |
| | 35, 36, 37, 38, 39, 40, 41, 42, 48, 49, 50, 51, 52, 53, 54, 55, 56, 57, 58, |
| | 59, 60, 61, 62, 63, 64, 65, 66, 67, 68, 69, 70, 71 in WO2015150526A2 |
| CAR and not gate 2 | Identifier 49 in US20160296562 |
| CD435 | Identifier 5 in EP3074419A2 |
| TOSO | Identifier 5, 4 in WO2015075468 |
| MUC1 | Identifier 5, 7 in WO2013063419; Identifier 51 in US20160340406A1; |
| | Identifier 30, 32, 34 in US20160130357; Identifier 295, 298, 301, 304, |
| | 307, 607, 609, 611, 613 in WO2016130726 |
| CAR and not gate 3 | Identifier 50 in US20160296562 |
| CD8 stalk APRIL | Identifier 51 in US20160296562A1 |
| HSP70 | Identifier 51, 53, 5 in WO2015077789; Identifier 21, 22, 23, 24, |
| | 25, 26, 27, 28, 29 in WO2016120217 |
| APRIL-based CAR | Identifier 53 in US20160296562A1; Identifier 52 in US20160296562A1 |
| CS1 | Identifier 55, 57, 60, 54, 56, 48, 49, 50, 51, 52, 53, 58, 59, 61, 62 |
| | in WO2015121454; Identifier 28 in WO2014179759A1 |
| CAR and not gate | Identifier 6 in US20160296562A1 |
| (CD19 and not CD33) | |
| Trophoblast | Identifier 6 in WO2015075468; Identifier 4 in |
| Glycoprotein 5T4 | US20160347854A1; Identifier 4 in EP3098237A1; Identifier 19, |
| | 20, 21, 22, 23, 24, 25, 26, 27, 28, 29, 30, 31, 32, 33, 34, 35, 36, 37, |
| | 38, 39 in WO2016034666A1 |
| HERVK | Identifier 6 in WO2016168769A1 |
| NCAR with RQR82 | Identifier 615 in WO2016130726 |
| ACD 19 | |
| NYBR1 | Identifier 617, 619 in WO2016130726; Identifier 218 in |
| | WO2016097231; Identifier 26, 29, 60 in WO2015112830; |
| | Identifier 1 in US20160333422A1 |
| CD123 | Identifier 69 in WO2016142532; Identifier 23, 24, 25, 26, 27, 28, 29, |
| | 30, 31, 32, 33, 34, 35, 36, 37, 38, 39, 40, 41, 42, 44, 45, 46, 47, 48 in |
| | WO2015140268A1; Identifier 9, 10, 11, 12 in US20140271582; |
| | Identifier 56, 57, 58, 59, 60, 61 in WO2016097231; Identifier 98, 99, |
| | 100, 101, 125, 126, 127, 128, 129, 130, 131, 132, 133, 134, 135, 136, |
| | 137, 138, 139, 140, 141, 142, 143, 144, 145, 146, 147, 148, 149, 150, |
| | 151, 152, 153, 154, 155, 156 in WO2016028896; Identifier 31, 32, 33, |
| | 34, 35, 36, 37, 38, 39, 40, 41, 42, 43, 44, 45, 46, 47, 48, 49, 50, 51, 52, |
| | 53, 54, 55, 56, 57, 58, 59, 60, 61, 62, 63, 64, 65, 66, 67, 68, 69, 70, 71, |
| | 72, 73, 74, 75, 76, 77, 78, 79, 80, 81, 82, 83, 84, 85, 86, 87, 88, 89, 90, |
| | 91, 92, 93, 94, 95, 96, 97, 98, 99, 100, 101, 102, 103, 104, 105, 106, |
| | 107, 108, 109, 110, 111, 112, 113, 114, 115, 116, 117, 118, 119, 120, |
| | 121, 122, 123, 124, 125, 126, 127, 128, 129, 130, 131, 132, 133, 134, |
| | 135, 136, 137, 138, 139, 140, 141, 142, 143, 144, 145, 146, 147, 148, |
| | 149, 150, 151, 152, 153, 154, 155, 156, 157, 158, 159, 160, 172, 173, |
| | 174, 175, 176, 177, 178, 179, 180, 181, 182, 183, 184, 185, 186, 187, |
| | 188, 189, 190, 191, 193, 194, 195, 196, 197 in WO2016120220; |
| | Identifier 1, 2, 3, 4, 5, 6, 7, 8, 9, 10, 142 in WO2016120216 |
| CD410 | Identifier 7 in EP3074419A2 |
| CD38 | Identifier 70, 71, 72, 64, 65, 66, 67, 68, 69 in WO2016097231; |
| | Identifier 35, 36, 37 in WO2015121454 |
| GCN4 | Identifier 8, 10 in U.S. Pat. No. 9,446,105B2 |
| CD4-DDY3 | Identifier 9 in EP3074419A2 |
| Fra | Identifier 959 in WO2016090337; Identifier 13 in US20170002072A1 |
| CD70 | Identifier 99 in WO2015121454 |

In some embodiments, the antigen-binding moiety is a single-chain Fv (scFv). In some embodiments, the scFv comprises a linker between the VH and VL. Non-limiting examples of the linker sequence that may be used in the scFvs described herein include, GGGGSGGGGSGGGGS ((G4S) 3; SEQ ID NO: 12), GGGGS (SEQ ID NO: 15), GGGGSGGGGS ((G4S) 2; SEQ ID NO: 17), GGGGSGGGGSGGGGSGGGGS ((G4S) 4; SEQ ID NO: 18), KESGSVSSEQLAQFRSLD (SEQ ID NO: 19), EGKSSGSGSESKST (SEQ ID NO: 20), EGKSSGSGSESKSTQ (SEQ ID NO: 21), GSTSGSGKSSEGKG (SEQ ID NO: 22), SSADDAKKDDAKKDDAKKDDAKKDG (SEQ ID NO: 23), EGKSSGSGSESKVD (SEQ ID NO: 24), or ESGSVSSEELAFRSLD (SEQ ID NO: 25), or SGGGGSGGGGSGGGGS ((SG4)3 linker; SEQ ID NO: 104)) or a functional variant thereof. Additional linkers include those described in, e.g., Whitlow and Filpula, Methods, Volume 2, Issue 2, April 1991, Pages 97-105, the content of which is incorporated herein by reference in its entirety.

In some embodiments, the linker sequence comprises the amino acid sequence GGGGSGGGGSGGGGS (SEQ ID NO: 12), or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 12. In certain embodiments, the nucleotide sequence that encodes the linker sequence comprises the nucleotide sequence that encodes the amino acid sequence of SEQ ID NO: 12, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 12. In certain embodiments, the nucleotide sequence that encodes the linker sequence comprises the nucleotide sequence set forth in SEQ ID NO: 13, 14 or 101, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 13, 14, or 101. In certain embodiments, the linker sequence comprises the amino acid sequence set forth in SEQ ID NO: 12. In certain embodiments, the nucleotide sequence that encodes the linker sequence comprises the nucleotide sequence set forth in SEQ ID NO: 13, 14, or 101.

In some embodiments, the linker sequence comprises the amino acid sequence SGGGGSGGGGSGGGGS (SEQ ID NO: 104), or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 104. In certain embodiments, the nucleotide sequence that encodes the linker sequence comprises the nucleotide sequence that encodes the amino acid sequence of SEQ ID NO: 104, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 104. In certain embodiments, the nucleotide sequence that encodes the linker sequence comprises the nucleotide sequence set forth in SEQ ID NO: 105, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 105. In certain embodiments, the linker sequence comprises the amino acid sequence set forth in SEQ ID NO: 104. In certain embodiments, the nucleotide sequence that encodes the linker sequence comprises the nucleotide sequence set forth in SEQ ID NO: 105.

In certain embodiments, the antigen-binding moiety comprises a polypeptide or functional variant thereof that binds to EphA2. In certain embodiments, the antigen-binding moiety is a single chain variable fragment (scFv) that binds to a EphA2. In some embodiments, the anti-EphA2 scFv is derived from an mAb specific for the EphA2, or a functional variant thereof.

In some embodiments, EphA2 scFv comprises a heavy chain variable domain (VH) comprising the amino acid sequence set forth in SEQ ID NO: 94, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 94. In certain embodiments, the nucleotide sequence that encodes the VH of EphA2 scFv comprises the nucleotide sequence that encodes the amino acid sequence of SEQ ID NO: 94, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 94. In certain embodiments, the nucleotide sequence that encodes the VH of EphA2 scFv comprises the nucleotide sequence set forth in SEQ ID NO: 95, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 95. In certain embodiments, the VH of EphA2 scFv comprises the amino acid sequence set forth in SEQ ID NO: 94. In certain embodiments, the nucleotide sequence that encodes the VH of EphA2 scFv comprises the nucleotide sequence set forth in SEQ ID NO: 95.

In some embodiments, EphA2 scFv comprises a light chain variable domain (VL) comprising the amino acid sequence set forth in SEQ ID NO: 96, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 96. In certain embodiments, the nucleotide sequence that encodes the VL of EphA2 scFv comprises the nucleotide sequence that encodes the amino acid sequence of SEQ ID NO: 96, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 96. In certain embodiments, the nucleotide sequence that encodes the VL of EphA2 scFv comprises the nucleotide sequence set forth in SEQ ID NO: 97, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 97. In certain embodiments, the VL of EphA2 scFv comprises the amino acid sequence set forth in SEQ ID NO: 96. In certain embodiments, the nucleotide sequence that encodes the VL of EphA2 scFv comprises the nucleotide sequence set forth in SEQ ID NO: 97.

In some embodiments, EphA2 scFv comprises the amino acid sequence set forth in SEQ ID NO: 10, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 10. In certain embodiments, the nucleotide sequence that encodes the EphA2 scFv comprises the nucleotide sequence that encodes the amino acid sequence of SEQ ID NO: 10, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 10. In certain embodiments, the nucleotide sequence that encodes the EphA2 scFv comprises the nucleotide sequence set forth in SEQ ID NO: 11, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 11. In certain embodiments, the EPHA2 scFv comprises the amino acid sequence set forth in SEQ ID NO: 10. In certain embodiments, the nucleotide sequence that encodes the EphA2 scFv comprises the nucleotide sequence set forth in SEQ ID NO: 11.

In certain embodiments, the antigen-binding moiety comprises a polypeptide or functional variant thereof that binds to B7-H3. In certain embodiments, the antigen-binding moiety is a single chain variable fragment (scFv) that binds to a B7-H3. In some embodiments, the anti-B7-H3 scFv is derived from an mAb specific for the B7-H3, or a functional variant thereof.

In some embodiments, B7-H3 scFv comprises a heavy chain variable domain (VH) comprising the amino acid sequence set forth in SEQ ID NO: 102, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 102. In certain embodiments, the nucleotide sequence that encodes the VH of B7-H3 scFv comprises the nucleotide sequence that encodes the amino acid sequence of SEQ ID NO: 94, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 102. In certain embodiments, the nucleotide sequence that encodes the VH of B7-H3 scFv comprises the nucleotide sequence set forth in SEQ ID NO: 103, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 103. In certain embodiments, the VH of B7-H3 scFv comprises the amino acid sequence set forth in SEQ ID NO: 102. In certain embodiments, the nucleotide sequence that encodes the VH of B7-H3 scFv comprises the nucleotide sequence set forth in SEQ ID NO: 103.

In some embodiments, B7-H3 scFv comprises a light chain variable domain (VL) comprising the amino acid sequence set forth in SEQ ID NO: 106, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 106. In certain embodiments, the nucleotide sequence that encodes the VL of B7-H3 scFv comprises the nucleotide sequence that encodes the amino acid sequence of SEQ ID NO: 106, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 106. In certain embodiments, the nucleotide sequence that encodes the VL of B7-H3 scFv comprises the nucleotide sequence set forth in SEQ ID NO: 107, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 107. In certain embodiments, the VL of B7-H3 scFv comprises the amino acid sequence set forth in SEQ ID NO: 106. In certain embodiments, the nucleotide sequence that encodes the VL of B7-H3 scFv comprises the nucleotide sequence set forth in SEQ ID NO: 107.

In some embodiments, B7-H3 scFv comprises the amino acid sequence set forth in SEQ ID NO: 121, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 121. In certain embodiments, the nucleotide sequence that encodes the B7-H3 scFv comprises the nucleotide sequence that encodes the amino acid sequence of SEQ ID NO: 121, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 121. In certain embodiments, the nucleotide sequence that encodes the B7-H3 scFv comprises the nucleotide sequence set forth in SEQ ID NO: 122, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 122. In certain embodiments, the B7-H3 scFv comprises the amino acid sequence set forth in SEQ ID NO: 121. In certain embodiments, the nucleotide sequence that encodes the B7-H3 scFv comprises the nucleotide sequence set forth in SEQ ID NO: 122.

Further, the extracellular domain of CAR may comprise a domain that can be paired with multiple, antigen recognition domains (e.g., avidin-CARs/biotin-labeled scFvs, CD16-CAR/MAbs, anti-FITC-CARs/FITC-labeled scFv, coiled-coil CARs (SUPRA CARs), anti-PNE-CARs/PNE-scFv, and NKG2D-CARs/ULBP2-MAb). These CARs are also known as “universal CARs”.

### Leader Sequence

In certain aspects, the CAR of the present disclosure comprises a leader sequence. The leader sequence may be positioned amino-terminal to the extracellular target-binding domain. The leader sequence may be optionally cleaved from the antigen-binding moiety during cellular processing and localization of the CAR to the cellular membrane.

In some embodiments, the leader sequence may be derived CD8α or human immunoglobulin heavy chain variable region.

In some embodiments, the leader sequence may be derived from human immunoglobulin heavy chain variable region. In some embodiments, the leader sequence comprises the amino acid sequence set forth in SEQ ID NO: 5 or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 5. In certain embodiments, the nucleotide sequence encoding the leader sequence comprises the nucleotide sequence that encodes the amino acid sequence of SEQ ID NO: 5, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 5. In certain embodiments, the nucleotide sequence encoding the leader sequence comprises the sequence set forth in SEQ ID NO: 6, 7, or 100, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 6, 7, or 100. In certain embodiments, the leader sequence comprises the amino acid sequence of SEQ ID NO: 5. In certain embodiments, the nucleotide sequence encoding the leader sequence comprises the nucleotide sequence set forth in SEQ ID NO: 6, 7, or 100.

In some embodiments, the leader sequence may be derived from CD8α. In some embodiments, the leader sequence comprises the amino acid sequence set forth in SEQ ID NO: 8 or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 8. In certain embodiments, the nucleotide sequence encoding the leader sequence comprises the nucleotide sequence that encodes the amino acid sequence of SEQ ID NO: 8, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 8. In certain embodiments, the nucleotide sequence encoding the leader sequence comprises the sequence set forth in SEQ ID NO: 9, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 9. In certain embodiments, the leader sequence comprises the amino acid sequence of SEQ ID NO: 8. In certain embodiments, the nucleotide sequence encoding the leader sequence comprises the nucleotide sequence set forth in SEQ ID NO: 9.

### Hinge Domain

In certain embodiments, the CAR further comprises a hinge domain between the extracellular antigen-binding moiety and the transmembrane domain, wherein the antigen-binding moiety, linker, and the transmembrane domain are in frame with each other.

A hinge domain can comprise any oligo- or polypeptide that functions to link the antigen-binding moiety to the transmembrane domain. A hinge domain can be used to provide more flexibility and accessibility for the antigen-binding moiety. A hinge domain may comprise up to 300 amino acids, preferably 10 to 100 amino acids and most preferably 25 to 50 amino acids. A hinge domain may be derived from all or part of naturally occurring molecules, such as from all or part of the extracellular region of CD8, CD4 or CD28, or from all or part of an antibody constant region. Alternatively, the hinge domain may be a synthetic sequence that corresponds to a naturally occurring linker region sequence, or may be an entirely synthetic linker region sequence. Non-limiting examples of hinge domains which may be used in accordance with the disclosure include a part of human CD8α chain, partial extracellular domain of CD28, FcyRIIIa receptor, IgG, IgM, IgA, IgD, IgE, an Ig hinge, or functional fragment thereof. In some embodiments, additional linking amino acids are added to the linker region to ensure that the antigen-binding moiety is an optimal distance from the transmembrane domain. In some embodiments, when the hinge domain is derived from an Ig, the linker may be mutated to prevent Fc receptor binding.

In some embodiments, the hinge domain may be derived from CD8α, CD28, or an immunoglobulin (IgG). For example, the IgG hinge may be from IgG1, IgG2, IgG3, IgG4, IgM1, IgM2, IgA1, IgA2, IgD, IgE, or a chimera thereof.

In certain embodiments, the linker domain comprises an immunoglobulin IgG hinge or functional fragment thereof. In certain embodiments, the IgG hinge is from IgG1, IgG2, IgG3, IgG4, IgM1, IgM2, IgA1, IgA2, IgD, IgE, or a chimera thereof. In certain embodiments, the linker domain comprises the CH1, CH2, CH3 and/or hinge region of the immunoglobulin. In certain embodiments, the linker domain comprises the core hinge region of the immunoglobulin. The term “core hinge” can be used interchangeably with the term “short hinge” (a.k.a “SH”). Non-limiting examples of suitable linker domains are the core immunoglobulin hinge regions listed in Table 4 (see also Wypych et al., JBC 2008 283 (23): 16194-16205, which is incorporated herein by reference in its entirety for all purposes). In certain embodiments, the linker domain is a fragment of the immunoglobulin hinge.

TABLE 4
Amino Acid Sequence of Short Hinge Regions of IgG Immunoglobulins

| IgG Subtype | Short Hinge Sequence | SEQ ID NO |
|---|---|---|
| IgG1 | DLEPKSCDKTHTCPPCPDPK | SEQ ID NO: 26 |
| IgG1 | EPKSCDKTHTCPPCP | SEQ ID NO: 34 |
| IgG2 | ERKCCVECPPCP | SEQ ID NO: 35 |
| IgG3 | ELKTPLGDTTHTCPRCP(EPKSCDTPPPCPRCP)₃ | SEQ ID NO: 36 |
| IgG4 | ESKYGPPCPSCP | SEQ ID NO: 37 |

In certain embodiments, the hinge domain comprises an IgG1 hinge, or a variant thereof. In certain embodiments, the hinge domain comprises the short hinge structure of IgG1, IgG2, IgG3, or IgG4 or a variant thereof. In certain embodiments, hinge domain comprises a short hinge region and comprises the amino acid sequence set forth in SEQ ID NO: 26, 34, 35, 36, or 37, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 26, 34, 35, 36, or 37. In certain embodiments, the nucleotide sequence encoding the hinge comprising the short hinge region comprises the nucleotide sequence that encodes the amino acid sequence of SEQ ID NO: 26, 34, 35, 36, or 37, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 26, 34, 35, 36, or 37. In certain embodiments, hinge domain comprises a short hinge region and comprises the amino acid sequence set forth in SEQ ID NO: 26, 34, 35, 36, or 37.

In certain embodiments, hinge domain comprises a short hinge region and comprises the amino acid sequence set forth in SEQ ID NO: 26, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 26. In certain embodiments, the nucleotide sequence encoding the hinge comprising the short hinge region comprises the nucleotide sequence that encodes the amino acid sequence of SEQ ID NO: 26, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 26. In certain embodiments, hinge domain comprises a short hinge region and comprises the amino acid sequence set forth in SEQ ID NO: 26. In certain embodiments, the nucleotide sequence encoding the hinge comprising the short hinge region comprises the nucleotide sequence of SEQ ID NO: 27, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 27. In certain embodiments, the short hinge region comprises the amino acid sequence set forth in SEQ ID NO: 26. In certain embodiments, the nucleotide sequence that encodes the short hinge region comprises the nucleotide sequence set forth in SEQ ID NO: 27.

In some embodiments, the hinge domain is derived from IgG4. In some embodiments, the hinge domain derived from IgG4 comprises the amino acid sequence set forth in SEQ ID NO: 28, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 28. In certain embodiments, the nucleotide sequence that encodes the IgG4 hinge domain comprises the nucleotide sequence that encodes the amino acid sequence of SEQ ID NO: 28, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 28. In certain embodiments, the nucleotide sequence that encodes the IgG4 hinge domain comprises the nucleotide sequence set forth in SEQ ID NO: 29, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 29. In certain embodiments, the IgG4 hinge domain comprises the amino acid sequence set forth in SEQ ID NO: 28. In certain embodiments, the nucleotide sequence that encodes the IgG4 hinge domain comprises the nucleotide sequence set forth in SEQ ID NO: 29.

In some embodiments, the hinge domain is derived from CD8α stalk or complete or partial sequences of the CD8α stalk, which are also called CD8α hinge. In some embodiments, the hinge domain derived from CD8α stalk comprises the amino acid sequence set forth in SEQ ID NO: 30, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 30. In certain embodiments, the nucleotide sequence that encodes the CD8α stalk hinge domain comprises the nucleotide sequence that encodes the amino acid sequence of SEQ ID NO: 30, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 30. In certain embodiments, the nucleotide sequence that encodes the CD8α stalk hinge domain comprises the nucleotide sequence set forth in SEQ ID NO: 31 or 108, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 31 or 108. In certain embodiments, the CD8α stalk hinge domain comprises the amino acid sequence set forth in SEQ ID NO: 30. In certain embodiments, the nucleotide sequence that encodes the CD8α stalk hinge domain comprises the nucleotide sequence set forth in SEQ ID NO: 31 or 108.

In some embodiments, the hinge domain is derived from CD28. In some embodiments, the hinge domain derived from CD28 hinge domain comprises the amino acid sequence set forth in SEQ ID NO: 32, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 32. In certain embodiments, the nucleotide sequence that encodes the CD28 hinge domain comprises the nucleotide sequence that encodes the amino acid sequence of SEQ ID NO: 32, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 32. In certain embodiments, the nucleotide sequence that encodes the CD28 hinge domain comprises the nucleotide sequence set forth in SEQ ID NO: 33, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 33. In certain embodiments, the CD28 hinge domain comprises the amino acid sequence set forth in SEQ ID NO: 32. In certain embodiments, the nucleotide sequence that encodes the CD28 hinge domain comprises the nucleotide sequence set forth in SEQ ID NO: 33.

In some embodiments, in addition to the sequences described above, the hinge domain can comprise additional linker amino acids to allow for extra flexibility and/or accessibility.

### Transmembrane Domain

In certain aspects, the CARs of the present disclosure comprise a transmembrane domain, fused in frame between the extracellular target-binding domain and the cytoplasmic domain.

The transmembrane domain may be derived from the protein contributing to the extracellular target-binding domain, the protein contributing the signaling or co-signaling domain, or by a totally different protein. In some instances, the transmembrane domain can be selected or modified by amino acid substitution, deletions, or insertions to minimize interactions with other members of the CAR complex. In some instances, the transmembrane domain can be selected or modified by amino acid substitution, deletions, or insertions to avoid-binding of proteins naturally associated with the transmembrane domain. In certain embodiments, the transmembrane domain includes additional amino acids to allow for flexibility and/or optimal distance between the domains connected to the transmembrane domain.

The transmembrane domain may be derived either from a natural or from a synthetic source. Where the source is natural, the domain may be derived from any membrane-bound or transmembrane protein. Non-limiting examples of transmembrane domains of particular use in this disclosure may be derived from (i.e. comprise at least the transmembrane region(s) of) the α, β or ζ chain of the T-cell receptor, CD28, CD3 epsilon, CD45, CD4, CD5, CD8, CD8α, CD9, CD16, CD22, CD33, CD37, CD40, CD64, CD80, CD86, CD134, CD137, CD154. Alternatively, the transmembrane domain may be synthetic, in which case it will comprise predominantly hydrophobic residues such as leucine and valine. For example, a triplet of phenylalanine, tryptophan and/or valine can be found at each end of a synthetic transmembrane domain.

In some embodiments, the transmembrane domain may be derived from CD8α, CD28, CD8, CD4, CD3ζ, CD40, CD134 (OX-40), NKG2A/C/D/E, or CD7. In some embodiments, the transmembrane domain may be derived from CD28.

In certain embodiments, it will be desirable to utilize the transmembrane domain of the ζ, η or FcεR1γ chains which contain a cysteine residue capable of disulfide bonding, so that the resulting chimeric protein will be able to form disulfide linked dimers with itself, or with unmodified versions of the ζ, η or FcεR1γ chains or related proteins. In some instances, the transmembrane domain will be selected or modified by amino acid substitution to avoid-binding of such domains to the transmembrane domains of the same or different surface membrane proteins to minimize interactions with other members of the receptor complex. In other cases, it will be desirable to employ the transmembrane domain of ζ, η or FcΣR1γ and -β, MB1 (Igα.), B29 or CD3− γ, δ, or η, in order to retain physical association with other members of the receptor complex.

In certain embodiments, the transmembrane domain in the CAR of the disclosure is derived from the CD28 transmembrane domain. In certain embodiments, the CD28 transmembrane domain comprises the amino acid sequence set forth in SEQ ID NO: 38, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 38. In certain embodiments, the nucleotide sequence that encodes the CD28 transmembrane domain comprises the nucleotide sequence that encodes the amino acid sequence of SEQ ID NO: 38, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 38. In certain embodiments, the nucleotide sequence that encodes the CD28 transmembrane domain comprises the nucleotide sequence set forth in SEQ ID NO: 39, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 39. In certain embodiments, the CD28 transmembrane domain comprises the amino acid sequence set forth in SEQ ID NO: 38. In certain embodiments, the nucleotide sequence that encodes the CD28 transmembrane domain comprises the nucleotide sequence set forth in SEQ ID NO: 39.

In certain embodiments, the transmembrane domain in the CAR of the disclosure is derived from the CD28 transmembrane domain. In certain embodiments, the CD28 transmembrane domain comprises the amino acid sequence set forth in SEQ ID NO: 40, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 40. In certain embodiments, the nucleotide sequence that encodes the CD28 transmembrane domain comprises the nucleotide sequence that encodes the amino acid sequence of SEQ ID NO: 40, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 40. In certain embodiments, the nucleotide sequence that encodes the CD28 transmembrane domain comprises the nucleotide sequence set forth in SEQ ID NO: 41, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 41. In certain embodiments, the CD28 transmembrane domain comprises the amino acid sequence set forth in SEQ ID NO: 40. In certain embodiments, the nucleotide sequence that encodes the CD28 transmembrane domain comprises the nucleotide sequence set forth in SEQ ID NO: 41.

In certain embodiments, the transmembrane domain in the CAR of the disclosure is derived from the CD8α transmembrane domain. In certain embodiments, the CD8α transmembrane domain comprises the amino acid sequence set forth in SEQ ID NO: 38, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 38. In certain embodiments, the nucleotide sequence that encodes the CD8α transmembrane domain comprises the nucleotide sequence that encodes the amino acid sequence of SEQ ID NO: 38, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 38. In certain embodiments, the nucleotide sequence that encodes the CD8α transmembrane domain comprises the nucleotide sequence set forth in SEQ ID NO: 39, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 39. In certain embodiments, the CD8α transmembrane domain comprises the amino acid sequence set forth in SEQ ID NO: 38. In certain embodiments, the nucleotide sequence that encodes the CD8α transmembrane domain comprises the nucleotide sequence set forth in SEQ ID NO: 39.

In certain embodiments, the CD8α transmembrane domain comprises the amino acid sequence set forth in SEQ ID NO: 111, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 111. In certain embodiments, the nucleotide sequence that encodes the CD8α transmembrane domain comprises the nucleotide sequence that encodes the amino acid sequence of SEQ ID NO: 111, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 111. In certain embodiments, the nucleotide sequence that encodes the CD8α transmembrane domain comprises the nucleotide sequence set forth in SEQ ID NO: 112, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 112. In certain embodiments, the CD8α transmembrane domain comprises the amino acid sequence set forth in SEQ ID NO: 111. In certain embodiments, the nucleotide sequence that encodes the CD8α transmembrane domain comprises the nucleotide sequence set forth in SEQ ID NO: 112.

In certain embodiments, the transmembrane domain in the CAR of the disclosure is derived from the CD3ζ transmembrane domain. In certain embodiments, the CD33 transmembrane domain comprises the amino acid sequence set forth in SEQ ID NO: 44, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 44. In certain embodiments, the nucleotide sequence that encodes the CD8α transmembrane domain comprises the nucleotide sequence that encodes the amino acid sequence of SEQ ID NO: 44, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 44. In certain embodiments, the nucleotide sequence that encodes the CD8α transmembrane domain comprises the nucleotide sequence set forth in SEQ ID NO: 45, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 45. In certain embodiments, the CD8α transmembrane domain comprises the amino acid sequence set forth in SEQ ID NO: 44. In certain embodiments, the nucleotide sequence that encodes the CD8α transmembrane domain comprises the nucleotide sequence set forth in SEQ ID NO: 45.

### Cytoplasmic Domain

### Costimulatory and Signaling Domain

In certain aspects, CARs of the present disclosure comprise a cytoplasmic domain, which comprises one or more costimulatory domains and one or more signaling domains. The cytoplasmic domain, which comprises one or more costimulatory domains and one or more signaling domains, is responsible for activation of at least one of the normal effector functions of the lymphocyte in which the CAR has been placed in. The term “effector function” refers to a specialized function of a cell. Effector function of a T-cell, for example, may be cytolytic activity or helper activity including the secretion of cytokines. Thus, the term “signaling domain” refers to the portion of a protein which transduces the effector function signal and directs the cell to perform a specialized function. While usually the entire signaling domain is present, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the signaling domain sufficient to transduce the effector function signal.

Non-limiting examples of signaling domains which can be used in the CARs of the present disclosure include, e.g., signaling domains derived from DAP10, DAP12, Fc epsilon receptor I γ chain (FCER1G), FcR β, CD3δ, CD3ε, CD3γ, CD3ζ, CD5, CD22, CD226, NKG2D, CD66d, CD79A, and CD79B. In some embodiments, the CAR of the present disclosure comprises a signaling domain derived from CD3ζ.

In certain embodiments, the lymphocyte activation domain in the CAR of the disclosure is designed to comprise the signaling domain of CD3ζ. In certain embodiments, the CD3ζ signaling domain comprises the amino acid sequence set forth in SEQ ID NO: 60 or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 60. In certain embodiments, the nucleotide sequence that encodes the CD33 signaling domain comprises the nucleotide sequence that encodes the amino acid sequence of SEQ ID NO: 60, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 60. In certain embodiments, the nucleotide sequence that encodes the CD33 signaling domain comprises the nucleotide sequence set forth in SEQ ID NO: 61 or 110, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 61 or 110. In certain embodiments, the CD3ζ signaling domain comprises the amino acid sequence set forth in SEQ ID NO: 60. In certain embodiments, the nucleotide sequence that encodes the CD3ζ signaling domain comprises the nucleotide sequence set forth in SEQ ID NO: 61 or 110.

In certain embodiments, the CD33 signaling domain comprises the amino acid sequence set forth in SEQ ID NO: 62 or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 62. In certain embodiments, the nucleotide sequence that encodes the CD33 signaling domain comprises the nucleotide sequence that encodes the amino acid sequence of SEQ ID NO: 62, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 62. In certain embodiments, the nucleotide sequence that encodes the CD3ζ signaling domain comprises the nucleotide sequence set forth in SEQ ID NO: 61, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 63. In certain embodiments, the CD3ζ signaling domain comprises the amino acid sequence set forth in SEQ ID NO: 63. In certain embodiments, the nucleotide sequence that encodes the CD3ζ signaling domain comprises the nucleotide sequence set forth in SEQ ID NO: 62.

Non-limiting examples of costimulatory domains which can be used in the CARs of the present disclosure include, those derived from 4-1BB (CD137), CD28, CD40, ICOS, CD134 (OX-40), BTLA, CD27, CD30, GITR, CD226, CD79A, HVEM, MyD88, IL-2Rβ, or the STAT3-binding YXXQ. In some embodiments, the CAR of the present disclosure comprises one costimulatory domain. In some embodiments, the CAR of the present disclosure comprises a costimulatory domain derived from CD28.

In some embodiments, the costimulatory domains which can be used in the CARs of the present disclosure may be derived from CD28, 4-1BB, CD27, CD40, CD134, CD226, CD79A, ICOS, or MyD88, or any combination thereof.

In some embodiments, the CAR of the present disclosure comprises two or more costimulatory domains. In certain embodiments, the CAR of the present disclosure comprises two, three, four, five, six or more costimulatory domains. For example, the CAR of the present disclosure may comprise a costimulatory domain derived from 4-1BB and a costimulatory domain derived from CD28.

In certain embodiments, the CARs of the present disclosure comprise a cytoplasmic domain, which comprises a signaling domain, a MyD88 polypeptide or functional fragment thereof, and a CD40 cytoplasmic polypeptide region or a functional fragment thereof. In certain embodiments, the CAR lacks the CD40 transmembrane and/or CD40 extracellular domains. In certain embodiments, the CAR includes the CD40 transmembrane domain. In certain embodiments, the CAR includes the CD40 transmembrane domain and a portion of the CD40 extracellular domain, wherein the CD40 extracellular domain does not interact with natural or synthetic ligands of CD40.

In certain embodiments, the signaling domain is separated from the MyD88 polypeptide or functional fragment thereof and/or the CD40 cytoplasmic polypeptide region or a functional fragment thereof. In certain embodiments, the lymphocyte activation domain is separated from the MyD88 polypeptide or functional fragment thereof and/or the CD40 cytoplasmic polypeptide region or a functional fragment thereof by at least 1, 2, 3, 4, 5, 6, 7, 8, 9, 10, 11, 12, 13, 14, or 15 amino acids.

In some embodiments, the signaling domain(s) and costimulatory domain(s) can be in any order. In some embodiments, the signaling domain is upstream of the costimulatory domains. In some embodiments, the signaling domain is downstream from the costimulatory domains. In the cases where two or more costimulatory domains are included, the order of the costimulatory domains could be switched.

In some embodiments, the costimulatory domain derived from CD28 comprises the amino acid sequence set forth in SEQ ID NO: 46, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 46. In certain embodiments, the nucleotide sequence that encodes the CD28 costimulatory domain comprises the nucleotide sequence that encodes the amino acid sequence of SEQ ID NO: 46, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 46. In certain embodiments, the nucleotide sequence that encodes the CD28 costimulatory domain comprises the nucleotide sequence set forth in SEQ ID NO: 47 or 109, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 47 or 109. In certain embodiments, the CD28 costimulatory domain comprises the amino acid sequence set forth in SEQ ID NO: 46. In certain embodiments, the nucleotide sequence that encodes the CD28 costimulatory domain comprises the nucleotide sequence set forth in SEQ ID NO: 47 or 109.

In some embodiments, the costimulatory domain derived from 4-1BB comprises the amino acid sequence set forth in SEQ ID NO: 48, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 48. In certain embodiments, the nucleotide sequence that encodes the 4-1BB costimulatory domain comprises the nucleotide sequence that encodes the amino acid sequence of SEQ ID NO: 48, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 48. In certain embodiments, the nucleotide sequence that encodes the 4-1BB costimulatory domain comprises the nucleotide sequence set forth in SEQ ID NO: 49, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 49. In certain embodiments, the 4-1BB costimulatory domain comprises the amino acid sequence set forth in SEQ ID NO: 48. In certain embodiments, the nucleotide sequence that encodes the 4-1BB costimulatory domain comprises the nucleotide sequence set forth in SEQ ID NO: 49.

In some embodiments, the costimulatory domain derived from OX40 comprises the amino acid sequence set forth in SEQ ID NO: 50, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 50. In certain embodiments, the nucleotide sequence that encodes the OX40 costimulatory domain comprises the nucleotide sequence that encodes the amino acid sequence of SEQ ID NO: 50, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 50. In certain embodiments, the nucleotide sequence that encodes the OX40 costimulatory domain comprises the nucleotide sequence set forth in SEQ ID NO: 51, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 51. In certain embodiments, the OX40 costimulatory domain comprises the amino acid sequence set forth in SEQ ID NO: 50. In certain embodiments, the nucleotide sequence that encodes the OX40 costimulatory domain comprises the nucleotide sequence set forth in SEQ ID NO: 51.

In certain embodiments, the MyD88 polypeptide or functional fragment thereof in the CAR of the disclosure is designed to comprise the co-stimulatory domain of MyD88, or variant thereof. In certain embodiments, the MyD88 functional fragment comprises the amino acid sequence set forth in SEQ ID NO: 52, 54, or 56, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 52, 54, or 56. In certain embodiments, the nucleotide sequence encoding the MyD88 functional fragment comprises the nucleotide sequence that encodes the amino acid sequence of SEQ ID NO: 52, 54, or 56, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 52, 54, or 56. In certain embodiments, the nucleotide sequence encoding the MyD88 functional fragment comprises the nucleotide sequence set forth in SEQ ID NO: 53, 55, or 57, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 53, 55, or 57. In certain embodiments, the MyD88 functional fragment comprises the amino acid sequence set forth in SEQ ID NO: 52, 54, or 56. In certain embodiments, the nucleotide sequence that encodes the MyD88 functional fragment comprises the nucleotide sequence set forth in SEQ ID NO: 53, 55, 57.

In certain embodiments, the CD40 polypeptide or functional fragment thereof in the CAR of the disclosure is designed to comprise the CD40 cytoplasmic polypeptide region. In certain embodiments, the CD40 cytoplasmic polypeptide region comprises the amino acid sequence set forth in SEQ ID NO: 58 or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 58. In certain embodiments, the nucleotide sequence encoding the CD40 cytoplasmic polypeptide region comprises the nucleotide sequence that encodes the amino acid sequence of SEQ ID NO: 58, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 58. In certain embodiments, the nucleotide sequence encoding the CD40 cytoplasmic polypeptide region comprises the nucleotide sequence set forth in SEQ ID NO: 59, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 59. In certain embodiments, the CD40 cytoplasmic polypeptide region comprises the amino acid sequence of SEQ ID NO: 58. In certain embodiments, the nucleotide sequence encoding the CD40 cytoplasmic polypeptide region comprises the nucleotide sequence set forth in SEQ ID NO: 59.

### Anchoring Domain

In certain aspects, CARs of the present disclosure comprise a cytoplasmic domain, which comprises one or more anchoring domains. In some embodiments, the anchoring domain may be located at the C-terminal position of the CAR. In some embodiments, the anchoring domain may bind to a cell polarity protein, which may be capable of regulating or modifying spatial differences in shape, structure, and/or function within a cell, e.g., a eukaryotic cell, including an immune cell. By way of a non-limiting example, the cell polarity protein comprising the anchoring domain of the CAR disclosed herein may participate in synapse formation, migration, organization, and replication. In some embodiments, the cell polarity protein may comprise a Postsynaptic density-95, Discs large, and Zona occludens 1 (PDZ) domain. In some embodiments the anchoring domain may comprise a PDZ binding motif (PDZbm), which may be capable of binding a cell polarity protein comprising a PDZ domain. In some embodiments, the PDZbm may bind to Scribble.

In some embodiments, the cell polarity protein may comprise a PDZ domain. The PDZ domain may be derived from any of various classes of PDZ domains. Without wishing to be bound by theory, the PDZ domain may be derived from: (1) class I, which may recognize the motif S/T-X-Φ; (2) class II domains, which may recognize the motif Φ-X-Φ; and (3) class III domains, which may recognize the motif D/E-X-Φ as their preferred C-terminal motif, where Φ represents a hydrophobic residue (see, Lee and Zheng, Cell Communication and Signaling volume 8, Article number: 8 (2010), which is incorporated hereby by reference in its entirety).

Non-limiting examples of human PDZ domain-containing proteins include AAG12, AHNAK, AHNAK2, AIP1, ALP, APBA1, APBA2, APBA3, ARHGAP21, ARHGAP23, ARHGEF11, ARHGEF12, CARD10, CARD11, CARD14, CASK, CLP-36, CNKSR2, CNKSR3, CRTAM, DFNB31, DLG1, DLG2, DLG3, DLG4, DLG5, DVL1, DVL1L1, DVL2, DVL3, ERBB2IP, FRMPD1, FRMPD2, FRMPD2L1, FRMPD3, FRMPD4, GIPC1, GIPC2, GIPC3, GOPC, GRASP, GRIP1, GRIP2, HTRA1, HTRA2, HTRA3, HTRA4, IL16, INADL, KIAA1849, LDB3, LIMK1, LIMK2, LIN7A, LIN7B, LIN7C, LMO7, LNX1, LNX2, LRRC7, MAGI1, MAGI2, MAGI3, MAGIX, MAST1, MAST2, MAST3, MAST4, MCSP, MLLT4, MPDZ, MPP1, MPP2, MPP3, MPP4, MPP5, MPP6, MPP7, MYO18A, NHERF1, NOS1, PARD3, PARD6A, PARD6B, PARD6G, PDLIM1, PDLIM2, PDLIM3, PDLIM4, PDLIM5, PDLIM7, PDZD11, PDZD2, PDZD3, PDZD4, PDZD5A, PDZD7, PDZD8, PDZK1, PDZRN3, PDZRN4, PICK1, PPP1R9A, PPP1R9B, PREX1, PRX, PSCDBP, PTPN13, PTPN3, PTPN4, RAPGEF2, RGS12, RGS3, RHPN1, RIL, RIMS1, RIMS2, SCN5A, SCRIB (Scribble), SDCBP, SDCBP2, SHANK1, SHANK2, SHANK3, SHROOM2, SHROOM3, SHROOM4, SIPA1, SIPA1L1, SIPA1L2, SIPA1L3, SLC9A3R1, SLC9A3R2, SNTA1, SNTB1, SNTB2, SNTG1, SNTG2, SNX27, SPAL2, STXBP4, SYNJ2BP, SYNPO2, SYNPO2L, TAX1BP3, TIAM1, TIAM2, TJP1, TJP2, TJP3, TRPC4, TRPC5, USH1C, and WHEN.

In some embodiments, the PDZbm may be derived from any of the 16 classes as defined by the following C-terminal motifs: 1a (φ[K/R]XSDV); 1b (Ω[R/K]ET [S/T/R/K]φ); 1c (φφETXL); 1d (ETXV); 1e (TWΨ); 1f (ΩΩTWΨ); 1g (φφφ[T/S][T/S]ΩΨ); 1h (φφ[D/E][T/S]WΨ); 2a (FDΩΩC); 2b (WXΩFDV); 2c (WΩφDΨ); 2d (φφX[E/D]φφφ); 2e (φφφφ); 2f ([D/E]φΩφ); 3a (WΩ[S/T]DWΨ); 4a (ΩφGWF); φ, hydrophobic (V, I, L, F, W, Y, M); Ω, aromatic (F, W, and Y); Ψ, aliphatic (V, I, L, and M); and X, nonspecific.

In some embodiments, the PDZbm may be derived from Regulatory T cell Associated Molecule (CRTAM). In some embodiments, the CRTAM PDZbm may bind to a Scribble PDZ domain such as, but not limited to, the third PDZ domain of Scribble. In some embodiments, the CRTAM PDZbm that may bind to the third PDZ domain of Scribble may comprise the sequence of ESIV (SEQ ID NO: 1).

Non-limiting examples of binding motif, e.g., PDZbm, sequences that may be used in the anchoring domain of any of the PDZ CAR disclosed herein may be derived from any of the PDZ binding proteins described in Table 5. Ensemble ID (ENSP00000 ######) and the C-terminal 5 amino-acid motif for each PDZ binding protein are provided. Depending on pattern of the amino acids at the first, second, third, fourth, and fifth positions in the C-terminal motif, the PDZ binding proteins can be classified into 16 classes: 1a (q [K/R]XSDV); 1b (Ω[R/K]ET [S/T/R/K]φ); 1c (φφETXL); 1d (ETXV); 1e (TWΨ); 1f (ΩΩTWΨ); 1g (φφφ[T/S][T/S]ΩΨ); 1h (φφ[D/E][T/S]WΨ); 2a (FDΩΩC); 2b (WXΩFDV); 2c (WΩφDΨ); 2d (φφX[E/D]φφφ); 2e (φφφφ); 2f ([D/E]φΩφ); 3a (WΩ[S/T]DWΨ); 4a (ΩφGWF); φ, hydrophobic (V, I, L, F, W, Y, M); 22, aromatic (F, W, and Y); Y, aliphatic (V, I, L, and M); and X, nonspecific (Tonikian, R. et al. PLoS Biol 6, e239-e239, which is incorporated herein by reference in its entirety).

TABLE 5
Exemplary PDZ binding proteins

| | Amino | | Amino | | Amino | | Amino |
|---|---|---|---|---|---|---|---|
| Ensemble | Acid | Ensemble | Acid | Ensemble | Acid | Ensemble | Acid |
| ID | Sequence | ID | Sequence | ID | Sequence | ID | Sequence |
| 000233 | ELSKR | 288745 | QDKQS | 332915 | QFFRL | 362174 | QHPSE |
| 000412 | HLLPM | 288757 | AIPAN | 332931 | HIKGF | 362181 | DEHTL |
| 000442 | EAMMD | 288774 | LRHYR | 332932 | YMKGG | 362185 | APVFP |
| 001008 | VETEA | 288828 | ILRTD | 332958 | QDQNN | 362200 | EVYLI |
| 001146 | LSATV | 288840 | LNNPR | 332962 | HVAML | 362202 | LFGLQ |
| 002125 | ELAWQ | 288861 | HFWGC | 332963 | LPFLS | 362205 | GLRDT |
| 002165 | LTNVI | 288873 | QRSWV | 332967 | SNSYW | 362206 | ETLGH |
| 002596 | TFDWH | 288893 | DLAYT | 332969 | LRPFF | 362207 | TFWVT |
| 002829 | HPPDT | 288911 | ECSKV | 332973 | CSSVS | 362208 | LKDSQ |
| 003084 | QDTRL | 288912 | GQDGQ | 332974 | KWTPS | 362212 | KASRT |
| 003100 | KRRSK | 288937 | QTPGI | 332975 | LILVM | 362217 | QLYSA |
| 003302 | TVTVK | 288943 | QVLCH | 332979 | RHRWK | 362219 | QTPSS |
| 003607 | SGLHW | 288955 | TVNSE | 332991 | LNFLL | 362222 | RYLRK |
| 004103 | EVSGI | 288976 | PKPGL | 332995 | PWLKH | 362227 | SDSQY |
| 004921 | LKLNA | 288985 | DILEV | 333001 | DRRRR | 362230 | RVLLR |
| 004980 | HRKLL | 288986 | VKHLS | 333003 | RAILY | 362232 | APDEL |
| 004982 | PAAAK | 289004 | VVPGM | 333011 | SISDG | 362233 | GDHPG |
| 005082 | ESIHT | 289013 | HPGWP | 333014 | CADSF | 362235 | SQSGP |
| 005178 | KEVAM | 289014 | RMFAV | 333018 | VAKNY | 362238 | SLGHF |
| 005180 | TPKQL | 289081 | LRTQV | 333019 | MIQLF | 362244 | VTEVL |
| 005198 | QEFWS | 289094 | QQYLA | 333024 | CKKSK | 362255 | TEKSD |
| 005226 | NQIHR | 289104 | QCVIL | 333037 | HQRCF | 362261 | PAGEE |
| 005257 | RCCIL | 289105 | AEDYA | 333041 | GLLRL | 362264 | YSTRS |
| 005260 | APIIR | 289153 | KDYRS | 333042 | SPCAC | 362265 | SPYEL |
| 005284 | RTTPV | 289166 | WLPCN | 333055 | CHKVE | 362266 | LKPKA |
| 005286 | IRGSS | 289226 | LGSAS | 333064 | QEKDE | 362273 | VFQKS |
| 005340 | FVDVM | 289228 | HRKTF | 333066 | EQPRL | 362285 | LVHLI |
| 005386 | KRYGG | 289248 | LSPAN | 333071 | SEALP | 362287 | TMYFA |
| 005558 | VGEFF | 289269 | DNSDQ | 333084 | PTRFI | 362294 | YLSSF |
| 005587 | EMYDI | 289290 | GMKRV | 333097 | SLPGV | 362296 | SCHDL |
| 005756 | LGLCD | 289292 | GPSNF | 333114 | DVDEE | 362299 | TSSMA |
| 005905 | FGRRK | 289316 | YTSSK | 333122 | DTLPF | 362300 | TMYFA |
| 005995 | LLGPV | 289352 | YGFGG | 333142 | LLLSP | 362304 | ASPVT |
| 006015 | ANPLL | 289359 | HTKNI | 333154 | SRVEA | 362306 | TMYFA |
| 006053 | VLVPV | 289361 | TICPA | 333157 | VLQQV | 362314 | FEMDI |
| 006101 | WSLLK | 289371 | VFEII | 333168 | VQAAQ | 362326 | KGLGS |
| 006275 | VIPKS | 289373 | CVQTS | 333170 | KKKRY | 362329 | HHQKN |
| 006526 | RFSSV | 289382 | TKMSK | 333181 | RVTSV | 362330 | NNDTN |
| 006658 | KLLHP | 289388 | RSGAA | 333183 | MLTCR | 362332 | AKHRK |
| 006724 | GMALI | 289407 | ADPWY | 333188 | SRLDL | 362335 | SQYID |
| 006777 | RVPMP | 289416 | EWKTI | 333190 | EMEVK | 362337 | RRIPQ |
| 006839 | KESLV | 289422 | DSAPL | 333193 | TSVWI | 362344 | PALSQ |
| 006951 | VWECR | 289429 | KRCFC | 333194 | SSSEA | 362352 | KLDAK |
| 007264 | LEPDS | 289431 | HLVYR | 333195 | PQRGI | 362353 | QASCS |
| 007390 | KRQRD | 289433 | WRWQS | 333196 | KKASK | 362354 | LDTLD |
| 007414 | GAVLW | 289448 | LTYRL | 333197 | DRCCC | 362362 | FERVF |
| 007510 | TRSYC | 289451 | FYPLS | 333199 | TDETL | 362363 | REEIL |
| 007516 | KDVYE | 289473 | LASAV | 333203 | KVNRP | 362366 | PTALL |
| 007633 | SCTLL | 289484 | ESPCT | 333208 | KFSAS | 362372 | MGWNY |
| 007660 | RPQMM | 289488 | KQNTL | 333210 | RRRLQ | 362381 | IAFFL |
| 007699 | TTILE | 289528 | NCSLC | 333212 | TISRR | 362382 | AFADP |
| 007708 | TYRVS | 289547 | NGRQF | 333221 | KAQGY | 362386 | YYEAV |
| 007722 | RDQYY | 289575 | EDSRV | 333223 | YHQVL | 362387 | LCYPN |
| 007735 | NTFGY | 289577 | AKKLV | 333234 | TPKKD | 362390 | INHYL |
| 007969 | EQSLI | 289619 | GEGQL | 333236 | GPVVK | 362392 | PLRPK |
| 008180 | PQSSK | 289630 | VSEFS | 333239 | GLGRR | 362395 | KICQE |
| 008311 | TRKQK | 289657 | DRSRS | 333251 | DLPHS | 362398 | LESTF |
| 008391 | TEKTD | 289672 | DIKDF | 333253 | EMIDS | 362399 | APTME |
| 008527 | RQSTN | 289707 | VQDLP | 333262 | GGLGP | 362401 | EICKV |
| 008938 | HYRSP | 289731 | LLSVT | 333266 | LHYEL | 362403 | THVEI |
| 009041 | PLLEL | 289746 | RGRTA | 333268 | DLQQV | 362408 | CVTLA |
| 009105 | VCLIM | 289749 | EGAED | 333275 | IGHSI | 362409 | VQTEF |
| 009120 | RQRRQ | 289753 | GIPTN | 333277 | RGERA | 362410 | PEFFL |
| 009180 | NREMV | 289779 | SSFLV | 333278 | SMLRK | 362413 | ALSNI |
| 009530 | GPAPL | 289788 | VAESS | 333283 | SLILL | 362424 | LTSRR |
| 009589 | TIADA | 289805 | PGARP | 333284 | EPTSF | 362432 | EWRNQ |
| 009606 | EALMV | 289816 | KPEPT | 333285 | RYRKH | 362446 | LHAGE |
| 010132 | LPPIV | 289820 | PGFKK | 333292 | ASVKV | 362447 | YPGDP |
| 010299 | AITSP | 289893 | PGPKR | 333293 | LYTKN | 362456 | GEKAM |
| 010404 | SKLYL | 289921 | PEGQV | 333298 | GYQTI | 362457 | FLDPT |
| 011292 | LNHPY | 289932 | WGYFA | 333300 | KIKMK | 362460 | NLTPI |
| 011473 | PPTGI | 289952 | QIQIG | 333310 | FRTEI | 362462 | GCDPT |
| 011619 | EDYLH | 289953 | MDPRR | 333311 | SVKRN | 362463 | MATLM |
| 011653 | TCSPI | 289957 | LHSYH | 333326 | EGEEF | 362465 | VETET |
| 011684 | SASEV | 289968 | ESTAL | 333329 | LLVCQ | 362469 | HFEGR |
| 011691 | SKAME | 289989 | NLSVG | 333337 | DARAP | 362472 | NGINF |
| 011700 | LELDS | 290015 | YTCKH | 333339 | STALK | 362479 | VCTAV |
| 011898 | KKYDA | 290075 | HRIFS | 333350 | TKSSQ | 362481 | YFGTD |
| 011989 | VEPLS | 290130 | ATCKS | 333354 | DRAAS | 362482 | NRQHQ |
| 012049 | EYLGL | 290144 | FVLYI | 333358 | LPTGQ | 362485 | IIDLK |
| 012134 | KSQLH | 290155 | KWRPR | 333363 | LCDIK | 362489 | GEGCS |
| 012443 | QLGMM | 290158 | LKNQA | 333367 | TDLKD | 362490 | NSRNP |
| 013070 | QYYCS | 290167 | LHTCR | 333374 | YSALR | 362493 | ETKSV |
| 013125 | HENSY | 290186 | NKAVR | 333376 | YERVL | 362499 | LALQR |
| 013222 | KKPGP | 290200 | QGPQS | 333394 | RNQAV | 362502 | IIGPA |
| 013807 | SGFRL | 290208 | TVPDS | 333395 | MSQRI | 362503 | PNENT |
| 013894 | CDTVD | 290216 | SQAYA | 333401 | VEFKR | 362504 | VPLPT |
| 014761 | KSSDS | 290231 | YQRHY | 333406 | GSQSG | 362524 | PNTFH |
| 014914 | KKEGS | 290246 | VYHYG | 333420 | INSNA | 362527 | RISLI |
| 014930 | WLLKT | 290271 | SHESA | 333424 | SMTAL | 362532 | LFSTD |
| 014935 | LCPAA | 290277 | ARLVP | 333433 | HMPDL | 362540 | PASIM |
| 015926 | PGETS | 290291 | KSGKP | 333434 | GIHTG | 362546 | LKELQ |
| 016171 | RRVPK | 290294 | GRKIP | 333438 | LPSAR | 362550 | QMHRA |
| 016913 | ANAPK | 290295 | NSATP | 333456 | FFPRK | 362555 | GEARV |
| 016946 | SRSSG | 290299 | MREIV | 333470 | SGECG | 362556 | TTEAN |
| 017003 | DGRLR | 290310 | FKMSP | 333478 | VCVRN | 362560 | EACSL |
| 019103 | RTSII | 290330 | REALP | 333482 | AFFTS | 362562 | EPLTL |
| 019317 | KETSI | 290341 | QARRK | 333484 | VAQMQ | 362566 | SASQF |
| 020673 | GRRKP | 290349 | RVEQW | 333490 | DLLRA | 362573 | SYFAS |
| 020926 | HQLHL | 290354 | VVQNW | 333491 | QTLGK | 362574 | DFSYS |
| 020945 | CCVAH | 290363 | HLLNL | 333496 | RVSAL | 362576 | GGNIS |
| 021763 | SETTV | 290374 | DSAYV | 333500 | QTLGG | 362578 | AKRLF |
| 021776 | LGMNN | 290377 | EEEVA | 333519 | MCGDH | 362586 | SDTSN |
| 023064 | EEDPE | 290378 | HRKCF | 333534 | CLRPR | 362587 | FPSKQ |
| 023165 | LDLRY | 290384 | EISST | 333535 | IWITD | 362589 | AMKAT |
| 023897 | PTPHQ | 290390 | APKPR | 333537 | DPTIP | 362592 | EGQGS |
| 023939 | TSYCF | 290399 | ITNGR | 333539 | MRTVI | 362594 | QKAKN |
| 024061 | LGRRQ | 290401 | QRNTN | 333547 | CCQNL | 362601 | IFRPD |
| 025008 | WNKKV | 290422 | PRCAR | 333551 | VAQEH | 362605 | VIIDI |
| 025301 | LLENA | 290426 | GIMRC | 333552 | EWHYL | 362606 | PGPSK |
| 025399 | PDVKA | 290429 | QEPPR | 333560 | AEKSA | 362607 | LMFAE |
| 025429 | DLPMS | 290438 | VMQDT | 333568 | RVSIL | 362613 | VIVSE |
| 025469 | RKSLM | 290470 | SEDFG | 333591 | ELPSP | 362616 | EDIDR |
| 026218 | GEVAL | 290472 | PRAQT | 333592 | LDDWK | 362617 | EECER |
| 026464 | NTSAS | 290510 | VREEL | 333593 | HPYFG | 362618 | EATLR |
| 027335 | KPLRS | 290524 | KATPP | 333595 | QRETL | 362620 | CSCLR |
| 028008 | KKTKH | 290536 | DPSRP | 333602 | KPPCQ | 362621 | EATLR |
| 029410 | WCTFS | 290541 | ISGFE | 333603 | ANSKA | 362624 | RALGI |
| 031135 | KEEKE | 290551 | MAVSS | 333627 | LRIVL | 362626 | GYEDE |
| 031146 | VGHQH | 290552 | GAEAG | 333633 | IVIED | 362629 | ISLLS |
| 033079 | SSKSI | 290559 | KSKYF | 333634 | SGPWP | 362634 | WLAST |
| 035307 | QANST | 290567 | FFCAP | 333638 | DPCDS | 362635 | GDLSD |
| 035383 | RELTV | 290573 | EAGQR | 333639 | GDLCV | 362637 | GVVGT |
| 037243 | NTFGF | 290575 | MEEED | 333657 | SKEIH | 362638 | AEQNE |
| 037502 | KLSKM | 290583 | ANRPY | 333660 | TRLHN | 362639 | VTVVD |
| 037869 | LSIGK | 290597 | YAYMQ | 333664 | EYPGN | 362640 | ICAMQ |
| 038176 | WKLQY | 290607 | IPLPD | 333666 | LAQTA | 362648 | TPYFL |
| 039007 | QKPKF | 290649 | KQQTS | 333667 | GFDEH | 362649 | EVKLA |
| 039989 | GRRSP | 290650 | NWQLL | 333680 | VYENL | 362650 | TIDDY |
| 040584 | EENKD | 290663 | HLQLW | 333684 | KMFCS | 362656 | RIPWS |
| 040663 | DGPQM | 290687 | VAGAP | 333685 | LEIEQ | 362663 | YQPQP |
| 040738 | KKAKR | 290702 | PPKSK | 333697 | RLSHS | 362667 | TLDDY |
| 040877 | GDTKP | 290705 | CSCCA | 333701 | TGFWN | 362674 | LKHVV |
| 042931 | EENLA | 290722 | PHFKH | 333711 | DVEPN | 362677 | RDPQT |
| 043402 | VLGPC | 290759 | SPVET | 333725 | RKTHK | 362678 | GWHHH |
| 044462 | KEKDK | 290765 | IARIP | 333727 | RLLRN | 362682 | LHFVV |
| 045065 | LEVHL | 290776 | NSEPA | 333735 | CFQLP | 362684 | RFTLC |
| 045083 | EITIF | 290780 | LKKSR | 333737 | LFSSP | 362687 | KRSFD |
| 046087 | AKTCL | 290786 | ISHIS | 333738 | PCKKR | 362688 | FEFEA |
| 046794 | AAGYP | 290795 | DDAWK | 333744 | PNTFV | 362690 | QAKQT |
| 052754 | LGNYK | 290818 | KSASS | 333746 | KKRKA | 362694 | LNWLL |
| 053243 | SISAR | 290823 | QQEQV | 333751 | GEANT | 362695 | AGVWD |
| 053468 | EESKS | 290846 | LSPLL | 333752 | RMKEI | 362696 | PELPS |
| 053867 | LRQLL | 290858 | KTTRP | 333769 | QRNSS | 362702 | WMSST |
| 054650 | PEQQS | 290866 | ELRHS | 333771 | SRTQT | 362706 | EFPQN |
| 054666 | WVVSS | 290871 | LTPWH | 333775 | GKSQV | 362707 | TKKKR |
| 054668 | WKYCV | 290881 | RLLPD | 333776 | PYKCE | 362710 | RRCPK |
| 054950 | NHDEL | 290894 | AIRTL | 333779 | PAELL | 362711 | TSARA |
| 055077 | APVAS | 290902 | PDNCV | 333796 | CMFFS | 362714 | VSSTR |
| 055335 | LAWFS | 290913 | AAHKG | 333799 | AKSFI | 362717 | LTNLF |
| 055682 | FQKDI | 290921 | ASDQL | 333800 | RSSLV | 362727 | WETFE |
| 056217 | VEQQA | 290942 | AKVKK | 333802 | IHTRV | 362728 | EGFQL |
| 056233 | KGKRK | 290949 | YIPIF | 333811 | HDILT | 362730 | CFALE |
| 057513 | KKVHP | 290953 | PCSRT | 333813 | LPRPS | 362734 | EERLI |
| 058691 | TKENE | 290968 | IAANS | 333821 | CSRYW | 362743 | TGDSR |
| 061240 | THTKK | 291009 | ILKVE | 333823 | GLEEE | 362744 | KQSSG |
| 062104 | QRAPN | 291011 | HNLAL | 333833 | HWQLA | 362746 | NGCHI |
| 064571 | LVFPL | 291041 | QQYNG | 333837 | TVVLE | 362748 | YNHWV |
| 064724 | KSAHV | 291050 | AEPLI | 333845 | VKTLD | 362751 | GKETC |
| 064778 | VPPHW | 291129 | LRARK | 333871 | NLEVR | 362758 | STCVN |
| 064780 | SNLVI | 291182 | TGGNL | 333873 | FKIVL | 362762 | FQTEV |
| 066014 | FPWNP | 291187 | ENPYK | 333887 | FSGCF | 362763 | RQSLL |
| 066544 | ESDEF | 291231 | KRSLT | 333896 | PLPPS | 362768 | ERANH |
| 070846 | HSLKD | 291232 | GPEQQ | 333900 | IDWED | 362777 | FQMAT |
| 071246 | EPMDP | 291281 | RISVL | 333902 | LVLSA | 362778 | LAGIF |
| 071281 | AKNAP | 291294 | ACSLC | 333915 | KTPQL | 362781 | ASPLG |
| 072516 | SLKNV | 291295 | MMTAK | 333917 | LMIPL | 362784 | GQGSQ |
| 072644 | AAKSS | 291358 | TEPPG | 333919 | ICVRS | 362787 | HVPDL |
| 072869 | PVCPP | 291374 | RSTVA | 333920 | GRWII | 362788 | DRIYR |
| 075120 | TTTNV | 291386 | TVCFY | 333926 | VAVKG | 362790 | RMKCQ |
| 075503 | NYSML | 291416 | NTVRI | 333932 | RMSCS | 362792 | KTFFL |
| 078429 | EYNLV | 291421 | LRSKI | 333934 | KSTQE | 362795 | SYSGL |
| 078527 | FLPWT | 291439 | QLRTQ | 333938 | IESKN | 362799 | IVPHV |
| 081029 | LLNVT | 291440 | TCTLR | 333940 | EEEEG | 362803 | AYPSG |
| 083182 | EGPSC | 291442 | YGSGQ | 333946 | AFASK | 362807 | ASRML |
| 084795 | RGYKN | 291458 | PPKNE | 333947 | AWKTT | 362810 | TTVLP |
| 084798 | VPHGR | 291481 | TPLHV | 333948 | EYFGS | 362814 | NRDFS |
| 085068 | NSLLH | 291495 | GRVRQ | 333950 | SVTTN | 362817 | YEKEA |
| 085219 | VILKH | 291525 | AECTF | 333952 | SGGCC | 362820 | SNERK |
| 086933 | PKGSC | 291526 | EDCHY | 333956 | EIKKR | 362824 | VTESA |
| 095634 | PTHHE | 291527 | EECEF | 333959 | PPPGR | 362841 | LYTSG |
| 155093 | EVGLP | 291536 | SDLQD | 333960 | KSDQP | 362846 | PNGII |
| 155840 | PDEGS | 291539 | EGDCA | 333964 | GEKQV | 362850 | SGSCQ |
| 155858 | PPSDT | 291547 | SDSLQ | 333980 | TGKRP | 362853 | WKSRR |
| 155926 | DPFFN | 291552 | RSGRF | 333993 | ICDTK | 362861 | ILFQY |
| 156084 | PPDKS | 291554 | SAPSS | 334003 | FGLTL | 362863 | PQVYF |
| 156109 | DTDDD | 291560 | LEHNV | 334008 | YRNVE | 362869 | TCRKG |
| 156471 | PEETK | 291565 | QATVL | 334021 | KEKKI | 362870 | ENTVV |
| 156476 | LKGPQ | 291567 | NPHKL | 334040 | DDILC | 362873 | YFWTK |
| 156499 | TMRDK | 291568 | ELTYF | 334042 | AQGSE | 362880 | RRGLQ |
| 156626 | AKAKN | 291569 | KKRRE | 334044 | AAADQ | 362881 | WLRVG |
| 156825 | EMEHV | 291572 | FKKKE | 334050 | NALPL | 362887 | KESMH |
| 157812 | HEFYK | 291574 | EVSVT | 334051 | QYELL | 362888 | PKVFP |
| 158009 | YLFPC | 291576 | EEMLA | 334052 | CLGHY | 362894 | LQAQV |
| 158762 | DLHTL | 291577 | ELTGK | 334053 | DHCGG | 362898 | GLIQT |
| 158771 | QRLGG | 291582 | APFPS | 334061 | MPHPH | 362900 | SHSRS |
| 159060 | NKESF | 291592 | IGPGP | 334068 | FSDGK | 362909 | MEVSC |
| 159087 | PSGWH | 291598 | VGFIS | 334072 | NPFSP | 362910 | IALPT |
| 159111 | PGAPF | 291634 | DGGQL | 334077 | GGVSL | 362918 | QGRGK |
| 160262 | PSRAE | 291654 | SRTTL | 334085 | AILTT | 362924 | AELAA |
| 160298 | GGTPK | 291672 | PLFAW | 334090 | PCRRT | 362934 | PGHFR |
| 160373 | EKPNK | 291688 | DMVDI | 334100 | FDTSA | 362935 | EAMGE |
| 160382 | ERKCP | 291691 | TPGPP | 334101 | PAAAS | 362936 | IPAMM |
| 160740 | AATGR | 291707 | INIPK | 334105 | LQNAN | 362937 | DKEQS |
| 160827 | RCGAS | 291715 | FPVCL | 334113 | LDVTL | 362942 | RYHRP |
| 160874 | PLLKF | 291759 | PWEQI | 334115 | ATKMS | 362946 | EGCGC |
| 161006 | AAARS | 291770 | GGRRL | 334122 | EASAH | 362948 | PKRLM |
| 161863 | KNTTD | 291823 | VTGHH | 334128 | FVNRK | 362952 | VRGMA |
| 162023 | EKTQQ | 291842 | NETSF | 334134 | VAKLS | 362959 | PCLAW |
| 162044 | HLAGS | 291860 | TDTSV | 334135 | SSISS | 362968 | KKMFF |
| 162391 | WDLIT | 291890 | NTQTL | 334140 | IERLK | 362978 | ELDFL |
| 162749 | PSLLR | 291892 | QPLPR | 334141 | LRNES | 362979 | SENPQ |
| 162863 | GPQSA | 291900 | MDTSR | 334143 | GCLCK | 362983 | LVEGN |
| 163059 | HQALD | 291901 | GGRWK | 334145 | PLNVS | 362987 | ISVGR |
| 163416 | QPYGK | 291906 | RFLEP | 334146 | PSQNS | 362991 | SPVKG |
| 164133 | SGGQS | 291934 | EEEED | 334148 | MKIWH | 362993 | VSGRW |
| 164139 | PDEAI | 291971 | SQINP | 334153 | TVEEN | 362994 | VETST |
| 164227 | APGGS | 292035 | DTCRQ | 334163 | LVKTP | 363001 | CTLVL |
| 164305 | MKMKF | 292077 | APYQP | 334170 | TERRT | 363003 | GTTPY |
| 165086 | IISRN | 292090 | KFLTE | 334176 | THYQK | 363006 | GAEEC |
| 165524 | SSQDG | 292114 | ELGEL | 334181 | WAPVP | 363008 | AWARK |
| 165698 | SSGTA | 292123 | FTRRY | 334183 | VTTES | 363015 | STDAE |
| 166139 | EENFV | 292125 | EILLL | 334186 | EESTM | 363018 | PQRLY |
| 166244 | PRRHL | 292140 | ENHAP | 334187 | SGGCC | 363027 | NQIIL |
| 166345 | LAAYI | 292144 | QLRRN | 334188 | ATAKA | 363031 | FQKTI |
| 166534 | STEVD | 292147 | QTPTA | 334197 | CVSLL | 363036 | WKEHG |
| 167106 | YQIRV | 292174 | SLTTF | 334201 | RLRGS | 363040 | HNYKA |
| 167218 | VTDTP | 292176 | AMESS | 334216 | LKIEK | 363041 | KKKET |
| 167586 | LRTKN | 292180 | RNSRT | 334219 | RRRGG | 363045 | SKKKK |
| 167588 | VEENI | 292199 | GSSGS | 334223 | TTFVR | 363046 | SILDN |
| 168148 | NTDFE | 292205 | YLCPV | 334225 | ITFFT | 363050 | VYKTK |
| 168216 | IRMQP | 292211 | PKEDG | 334228 | CTVLF | 363055 | DVNLP |
| 168712 | FLPRL | 292301 | DKEGA | 334238 | QASES | 363064 | SESGC |
| 168977 | QQDSM | 292303 | ISVGL | 334246 | AKKVC | 363071 | QHEVL |
| 169298 | RTIHC | 292309 | PTEAR | 334260 | HLCFL | 363079 | CEFPI |
| 169551 | RSQDD | 292314 | TCDSD | 334261 | NAKDS | 363081 | TCQRH |
| 169565 | AVFSL | 292327 | HIMSS | 334271 | SVILK | 363084 | RELLA |
| 170150 | LFYQS | 292330 | RRWEA | 334276 | KGKRD | 363089 | EATSI |
| 170168 | AKTKR | 292337 | GGNPG | 334277 | PSLGP | 363092 | WDIDF |
| 170447 | ESSEP | 292357 | RRQDR | 334280 | KMGKS | 363100 | EEDLD |
| 170564 | PLRRQ | 292369 | SVARL | 334285 | PKDRN | 363108 | KVSPF |
| 170630 | YMRVS | 292377 | LLGPR | 334287 | YSSCY | 363115 | PGDQT |
| 171111 | QNCTC | 292389 | SVKKS | 334294 | SMEIK | 363118 | EVVTI |
| 171214 | RVRPR | 292401 | PWEAS | 334300 | YRRCF | 363120 | PLYVF |
| 171757 | SSMIG | 292414 | TIRSR | 334304 | GKAKK | 363121 | FFFFF |
| 171887 | AGQKR | 292431 | AEALQ | 334305 | ELSAG | 363122 | LKQLF |
| 172196 | PGAVY | 292432 | QLTRV | 334308 | GPPPT | 363124 | LCCST |
| 172229 | ATSPV | 292433 | AVVAF | 334314 | QWRVI | 363126 | HHLLQ |
| 172853 | YVEAI | 292450 | EGEAP | 334319 | TEGYT | 363128 | EGIAD |
| 173229 | KCKKA | 292475 | LRKYH | 334329 | TDGDC | 363129 | TRPDR |
| 173527 | LLSKV | 292476 | FLSGQ | 334330 | CRQRI | 363131 | DDWSD |
| 173569 | ENDFS | 292478 | ALGGR | 334364 | GSIID | 363135 | PFGQS |
| 173785 | MKRHL | 292494 | LRFGP | 334373 | EGFQF | 363136 | SPSDV |
| 173898 | GFSYG | 292513 | GLSHF | 334375 | PAVVS | 363140 | EVVTI |
| 174618 | TLKLA | 292520 | FFRRL | 334379 | YSQKD | 363142 | YQTLI |
| 174653 | FQVRT | 292524 | DYFSL | 334382 | LLECS | 363149 | FVHPI |
| 175091 | PYLPA | 292530 | GPLAN | 334386 | ATEGN | 363157 | GAFLL |
| 175506 | SAVKA | 292539 | CCLLM | 334393 | SSVKF | 363161 | PFLAR |
| 175756 | EWTRV | 292543 | FQIQK | 334394 | YSSCT | 363162 | YRING |
| 176183 | LRACC | 292562 | QERHF | 334397 | HDGKH | 363165 | RKHVQ |
| 176195 | TLRPR | 292566 | QGRPV | 334405 | PGSHP | 363168 | PDECV |
| 176763 | SADAS | 292574 | SSADP | 334409 | FSEAC | 363175 | LAPRT |
| 177648 | EKPCT | 292579 | VHRKL | 334410 | LHLCC | 363189 | VFNLR |
| 177694 | NYFPN | 292586 | FDDDV | 334412 | KYACK | 363192 | VGWSC |
| 177742 | KGTPV | 292596 | LLPWA | 334415 | DDHHK | 363193 | IFGRY |
| 178638 | TEAHA | 292599 | LLGSQ | 334428 | MLQSI | 363205 | KWTQH |
| 178640 | QQGPP | 292609 | PFPSS | 334430 | GTERD | 363208 | PDECV |
| 179259 | LTETR | 292616 | IWGRM | 334437 | QGSGK | 363209 | CDKFY |
| 180166 | LLMYT | 292633 | NLYEC | 334441 | GRNIS | 363211 | LYKCT |
| 180173 | VFLTA | 292641 | LTVFG | 334448 | FCAIL | 363213 | EEGES |
| 181092 | QQMAP | 292644 | YMTYN | 334452 | ISILK | 363214 | WCLWG |
| 181383 | VIRNV | 292672 | PGHPY | 334456 | QVPFH | 363216 | EGKTF |
| 181796 | QAQES | 292728 | FALRR | 334458 | DIITA | 363221 | ASPQR |
| 181839 | RGLPY | 292729 | PNYHW | 334461 | LCCAS | 363226 | GTQPY |
| 182096 | DIEIL | 292748 | ITASL | 334463 | LGLML | 363229 | YYTRS |
| 182290 | RGLSD | 292778 | EPSLL | 334472 | LERYL | 363231 | PPGQG |
| 182377 | STLKV | 292779 | DEDGV | 334474 | LFENN | 363238 | WGQGS |
| 182527 | KLKSP | 292782 | KSTTV | 334495 | LQGRI | 363239 | SQASL |
| 183605 | KHDYV | 292807 | YETRC | 334499 | FWTKN | 363241 | KSNML |
| 184183 | RVQLE | 292808 | REGFL | 334501 | ISLLS | 363250 | EAMQL |
| 184266 | FHLSY | 292823 | EDEED | 334506 | FPSEL | 363254 | AEKSV |
| 184956 | GLTNQ | 292853 | RVRLS | 334514 | TVCFY | 363257 | VGGHP |
| 185150 | LSLPN | 292879 | PGSIM | 334523 | GRRHR | 363259 | TEGIS |
| 185206 | RLSRS | 292894 | RIPSA | 334538 | KTEAV | 363263 | SDHLS |
| 185885 | QKYAM | 292896 | AHKYH | 334540 | CVLCR | 363277 | PSTTL |
| 185907 | EVVTI | 292902 | GGWCG | 334547 | SHHIP | 363284 | EEVDN |
| 186436 | FPHEN | 292907 | SFPRN | 334553 | DFSSW | 363288 | WLKEE |
| 187397 | WQVKF | 292928 | TTGIQ | 334560 | SGYYY | 363296 | TNHAI |
| 187608 | DSSIS | 292964 | PYWGI | 334563 | KYLQK | 363298 | ISIVR |
| 187762 | AKKAD | 293062 | SKLDS | 334564 | TMKRQ | 363308 | EVAAF |
| 187830 | TEGTR | 293068 | RALLK | 334565 | LLSLS | 363313 | LWMAE |
| 187879 | FLNDI | 293190 | LESEV | 334575 | RAPGG | 363317 | STDIF |
| 188312 | EKFDI | 293195 | RLLGH | 334590 | KMTQH | 363322 | GSLEF |
| 188403 | IWDVQ | 293201 | EARPA | 334592 | CAERR | 363323 | EGKTC |
| 188790 | FSLSD | 293215 | QRFRH | 334611 | SKTGI | 363329 | TEHCH |
| 189444 | QPQVH | 293217 | LQSKL | 334612 | KARPE | 363330 | LFCFS |
| 190165 | LNTSS | 293218 | HTLQS | 334625 | DKQMS | 363338 | SPSPQ |
| 190839 | HQGPL | 293230 | PSSGP | 334632 | LSIKM | 363341 | LERAL |
| 190983 | QNSAF | 293233 | GAVSS | 334640 | LPSGC | 363344 | DKSHE |
| 191922 | IFSNF | 293255 | KMMSR | 334642 | PSESV | 363345 | DIIEK |
| 192314 | PFLGA | 293261 | SSNIS | 334644 | SGGCC | 363348 | EDHKA |
| 192788 | PFFEL | 293272 | SLEMS | 334650 | HLVKK | 363350 | LLTLD |
| 193322 | QENSN | 293273 | LPELD | 334657 | FAKLD | 363361 | FLKLA |
| 193391 | QVEEV | 293274 | GCRLR | 334660 | PTQKQ | 363362 | SSDTA |
| 193422 | PIQLS | 293275 | LLNSQ | 334662 | NLCPH | 363369 | TKQKK |
| 194097 | PIIQK | 293276 | DMKEN | 334665 | ALWEY | 363371 | RPPDL |
| 194118 | RLGFG | 293277 | KPYSI | 334668 | GSMRN | 363374 | FEPAP |
| 194130 | NETMP | 293280 | KTRKN | 334675 | QLNNN | 363376 | PCRRT |
| 194152 | SLVFS | 293288 | FSLRV | 334681 | NRKKH | 363381 | VPWLL |
| 194155 | SFEFT | 293303 | STLPV | 334685 | RHQVY | 363384 | MDTST |
| 194214 | SNLSS | 293308 | DVLPK | 334695 | EKSFD | 363386 | RESEV |
| 194530 | SYWEF | 293328 | PHAQS | 334696 | SSGFY | 363387 | SSWET |
| 194900 | SPEKL | 293330 | GQSGI | 334708 | SIAMI | 363390 | EEGHQ |
| 195419 | EEMCD | 293331 | QPPPV | 334711 | SSNII | 363391 | AQGPH |
| 195455 | TAVHQ | 293349 | GQLQD | 334714 | IEPST | 363394 | QGPSD |
| 195649 | IKDFL | 293350 | LPMGD | 334720 | NIEMQ | 363395 | VGSDP |
| 195654 | GMIKT | 293362 | AETLY | 334721 | RDVQD | 363397 | EILDV |
| 195656 | ALAKS | 293371 | LDSVL | 334726 | FHRPA | 363398 | SLEKP |
| 196061 | SFVDP | 293373 | AFHLN | 334741 | WETMT | 363399 | WSAVA |
| 196169 | PRARN | 293379 | ATSDA | 334743 | TVCFY | 363400 | HLSSL |
| 196371 | QQIAN | 293404 | SDPGS | 334754 | RLVKD | 363405 | RLRFF |
| 196482 | QPAEV | 293406 | AALSS | 334762 | SLITT | 363406 | STQPL |
| 196489 | RSNIV | 293414 | EGCIQ | 334767 | HTEKS | 363407 | PECQI |
| 196548 | IREST | 293422 | MVLNG | 334769 | QPHLL | 363414 | DQKTK |
| 196551 | AKSNR | 293431 | WLRTD | 334774 | MLILN | 363416 | IRHQF |
| 196968 | GNGKQ | 293471 | RICTE | 334781 | LCHKM | 363423 | TTDVS |
| 197268 | APYEI | 293493 | QSTAV | 334798 | PSKAP | 363431 | RSNPI |
| 198536 | SVLKA | 293502 | PADSV | 334800 | EPLVQ | 363433 | GAAPE |
| 198587 | QATLL | 293525 | SCKRC | 334801 | KKRKA | 363435 | NCISR |
| 198765 | KQQKQ | 293549 | LHECL | 334805 | QRTIQ | 363438 | KTECL |
| 198767 | QPSPL | 293599 | ELTTR | 334808 | YERIL | 363443 | RTTEP |
| 198801 | HLEFC | 293614 | PRPPE | 334813 | PWQAL | 363452 | AWGRH |
| 198939 | RDECK | 293618 | PRSPK | 334817 | NLPDI | 363456 | LERAL |
| 199280 | RGTKA | 293636 | VIASN | 334828 | VCTVV | 363457 | STDIF |
| 199320 | GIHFS | 293662 | EESQL | 334834 | YQFTC | 363458 | DLFSF |
| 199389 | DGGVG | 293664 | MPWHL | 334836 | FPSKQ | 363459 | LLTLD |
| 199447 | DPEEN | 293670 | GQGRH | 334840 | LKVQV | 363463 | QPLKK |
| 199448 | EDCSW | 293677 | RKRIF | 334844 | REDTL | 363465 | SSLGN |
| 199706 | RASGQ | 293686 | NVLIR | 334848 | EKSCD | 363478 | EDLWQ |
| 199708 | VSEYR | 293695 | RDGEC | 334849 | SETKL | 363489 | SCGCR |
| 199764 | RVALI | 293709 | YLNHP | 334851 | CLISS | 363495 | TSAPF |
| 199814 | KHSSP | 293722 | LPQSC | 334853 | MVLPL | 363500 | KRLTK |
| 199936 | KKKAT | 293725 | WRETI | 334854 | RRPRL | 363503 | NFMME |
| 200135 | LAKIK | 293739 | HKPNF | 334866 | QATCY | 363506 | NDEGL |
| 200181 | QFFQT | 293743 | VDTEW | 334868 | GYQLV | 363510 | VSPQS |
| 200453 | SGRRG | 293745 | KKASR | 334869 | SGGCC | 363511 | EIIHV |
| 200457 | VTTDC | 293756 | IQEGE | 334874 | WMGGQ | 363512 | NSVAI |
| 200557 | RSGGA | 293760 | SDSER | 334876 | LDNDG | 363516 | FDCAL |
| 200652 | LITAF | 293761 | NSVAI | 334879 | EEYSD | 363520 | HTKKE |
| 200676 | LQSLS | 293771 | DHKPT | 334883 | FQACL | 363524 | EEEAH |
| 200691 | CSCCQ | 293774 | LNKRR | 334890 | IMLIS | 363529 | SRFES |
| 201023 | ENNTH | 293777 | QMLEN | 334892 | RGRPA | 363532 | LVEAK |
| 201031 | EKHRK | 293778 | PDSNT | 334902 | LGLGS | 363533 | KTVES |
| 201586 | HPRPS | 293780 | PCIQP | 334910 | SLDAE | 363545 | LQEAV |
| 201647 | EMEVI | 293800 | THIET | 334934 | RAKLS | 363550 | THEHL |
| 201943 | GVYIR | 293804 | NFDQA | 334940 | ATHSN | 363554 | PTEGI |
| 201979 | NLAVL | 293805 | ILGGP | 334941 | GLESR | 363556 | QRLTL |
| 202017 | VILKG | 293813 | ARERL | 334952 | HETTV | 363558 | AMKCS |
| 202556 | QRTLA | 293826 | GFVKL | 334958 | GGPNP | 363559 | IDSLT |
| 202625 | VATAK | 293829 | SPPAP | 334960 | LPTSM | 363571 | GTVSV |
| 202677 | LSGTD | 293831 | VADLI | 334974 | IETTV | 363573 | KSSGH |
| 202773 | HKLVF | 293845 | QRVRI | 334985 | FSGFY | 363582 | NWPPE |
| 202788 | SHESQ | 293860 | HRWRD | 334991 | VKAYK | 363583 | LLNRA |
| 202816 | KQKVK | 293872 | EAGEI | 334992 | PLIYS | 363587 | RRTGF |
| 202831 | HLKSM | 293874 | LKELA | 334993 | PSLEA | 363590 | SEAVG |
| 202967 | LIDPC | 293879 | EVPGL | 334996 | ILQRI | 363591 | RFFKS |
| 203001 | PESDS | 293882 | STFLG | 334998 | CRCCA | 363596 | DLSDC |
| 203166 | QKLCS | 293886 | GGLTR | 335001 | FSGFY | 363597 | NRSYC |
| 203407 | FWLRF | 293889 | TSTKS | 335004 | ETPLL | 363603 | LTGVK |
| 203556 | AEDHL | 293892 | LTGQL | 335006 | SAGCC | 363614 | ATCKL |
| 203629 | EPEQL | 293894 | TLTRP | 335010 | RKLLP | 363616 | FLDSS |
| 203630 | RRYDW | 293897 | QTSKL | 335017 | QKSSV | 363617 | LSRDW |
| 203664 | AADKH | 293900 | GAPAR | 335024 | NPVAG | 363624 | ITVLP |
| 204005 | ARPRA | 293922 | LERCP | 335038 | LLLGH | 363635 | TQELM |
| 204279 | DNGWD | 293924 | EGEAL | 335040 | PTPRQ | 363638 | LLPPV |
| 204517 | DGELP | 293937 | DFGWG | 335041 | PISSW | 363640 | GGGIF |
| 204549 | AVKLH | 293968 | GLVRL | 335042 | PYRCE | 363641 | INELV |
| 204566 | SQEEQ | 293970 | DPDTQ | 335044 | SEMLQ | 363642 | EGQLQ |
| 204604 | EAEGS | 293973 | RCSAA | 335055 | NDWSD | 363643 | RADRL |
| 204615 | LSQEG | 293981 | PGKSL | 335060 | RSQEA | 363645 | KLFFL |
| 204637 | LLVEH | 293995 | QKLHL | 335062 | CSWPL | 363646 | LYGSA |
| 204679 | LRGSL | 294008 | KVERN | 335064 | PSFFI | 363647 | QANRK |
| 204726 | EGPGE | 294016 | VSKSV | 335074 | APADK | 363654 | EEDNE |
| 204961 | IYYKV | 294053 | VCNTI | 335076 | PSRHF | 363662 | RFSRR |
| 205061 | TGSPA | 294064 | QFKSN | 335082 | NLHLQ | 363663 | EKPCE |
| 205064 | LESTF | 294066 | HQSTY | 335083 | NSDMH | 363666 | GMECN |
| 205135 | APFHM | 294072 | LHDGE | 335085 | SKGKH | 363669 | GIIIK |
| 205143 | ILSVK | 294117 | FCALL | 335088 | ETTSN | 363670 | GMERN |
| 205194 | FSKDL | 294119 | REGAL | 335089 | GLRHL | 363671 | GMEMN |
| 205214 | GGNQK | 294129 | GEAPS | 335094 | CIEEI | 363672 | GMELK |
| 205402 | KSINF | 294161 | LAVVK | 335106 | EFGSC | 363673 | GNGLS |
| 205557 | ESGLV | 294168 | LYLPL | 335144 | NPTRK | 363676 | ILPGK |
| 205636 | EPLNA | 294172 | VAFMK | 335147 | GKRKC | 363678 | FRPRT |
| 205890 | EITLL | 294173 | YPFQL | 335153 | MEEVD | 363680 | EAPAS |
| 205948 | DVKPC | 294179 | VVFLA | 335156 | LKAPA | 363682 | GGGAN |
| 206020 | LPPTS | 294189 | TKASE | 335173 | SSVSE | 363683 | HNCEE |
| 206249 | FPATV | 294241 | DNHNL | 335181 | KSKTQ | 363689 | RSFCH |
| 206262 | SSSES | 294244 | KGNGV | 335185 | HCTMC | 363708 | GMNCL |
| 206380 | LATDG | 294247 | NELKL | 335193 | NPADT | 363709 | LMGTP |
| 206423 | HGYPY | 294258 | RVGPS | 335196 | TTLNQ | 363724 | ARALF |
| 206466 | RSGTR | 294288 | RMMSR | 335197 | QVKKK | 363727 | PETKL |
| 206474 | SKVYR | 294304 | CTDSS | 335201 | SFQKQ | 363729 | RWKFC |
| 206513 | VGGCS | 294309 | LWLCR | 335203 | LKHDD | 363734 | APGKA |
| 206542 | VTWRD | 294312 | PSFEK | 335216 | PAVHL | 363739 | LDIKW |
| 206544 | PNPAL | 294338 | RSTPM | 335223 | GGGCC | 363743 | GQQWK |
| 206595 | HYIGH | 294342 | HQQRC | 335229 | GLRTH | 363746 | DRFVR |
| 206660 | EDLFS | 294353 | QARLN | 335241 | PGDWH | 363747 | ILNVQ |
| 206765 | SRGGA | 294360 | QFVKC | 335243 | AGNIS | 363752 | CAEEN |
| 207437 | HILSV | 294383 | DDVDP | 335246 | LGISH | 363753 | TTATS |
| 207457 | QLELA | 294390 | HHNMM | 335247 | DTSLT | 363756 | AWLQS |
| 207549 | LRPAP | 294409 | VVLED | 335255 | NPYFK | 363761 | CSCGG |
| 207636 | SEAGP | 294413 | LAVGF | 335256 | VMRLG | 363762 | VSKKK |
| 207726 | PATRP | 294419 | SKEAY | 335261 | LGLQA | 363765 | VDFLF |
| 207771 | SGKFQ | 294428 | KKRVY | 335272 | LDELP | 363767 | HTYGL |
| 207870 | RGPPE | 294445 | TRLQK | 335273 | KKQQI | 363768 | LLFPD |
| 209540 | NRDME | 294454 | QHKGT | 335276 | RGGGT | 363772 | RNAEK |
| 209668 | TILMF | 294485 | SFINV | 335281 | CRQRI | 363774 | TFTPQ |
| 209718 | IQKHA | 294489 | GRYSP | 335285 | IIYPD | 363779 | LSLLD |
| 209728 | ATGLP | 294507 | PYSEV | 335292 | PETES | 363780 | CGEAK |
| 209873 | PHSHL | 294543 | STGLD | 335304 | LLLDL | 363787 | TKDPK |
| 209875 | ETAKS | 294600 | KGTSK | 335306 | SLQWE | 363792 | ETENS |
| 209884 | ESHIW | 294610 | KELLM | 335307 | GSIID | 363794 | GGLFM |
| 209929 | PCNSY | 294635 | QPPIE | 335309 | PSCVT | 363799 | IYSKC |
| 210060 | EKNED | 294638 | PISHT | 335320 | ANLPF | 363807 | IAHLE |
| 210187 | SCCRP | 294661 | AKKKK | 335321 | LDYIL | 363817 | PHQLA |
| 210313 | VEGAE | 294664 | YMEVM | 335325 | NNRLK | 363822 | YFHTQ |
| 210444 | KKIKS | 294665 | VLFIK | 335332 | LLATT | 363824 | TKKNE |
| 210633 | DESSV | 294671 | KKIIS | 335333 | GESDT | 363826 | PLSQV |
| 211076 | KVNGT | 294702 | QHGLK | 335339 | NSKGA | 363827 | RPCPS |
| 211122 | KIFRF | 294715 | SSTIK | 335347 | NELKL | 363832 | WNVPI |
| 211238 | ENKIY | 294725 | EETQL | 335357 | RSVSR | 363836 | PAIIS |
| 211287 | SGMKL | 294728 | QKSKV | 335358 | SGGCC | 363842 | NVVTQ |
| 211314 | VLLLL | 294736 | VLECK | 335369 | TGKGS | 363852 | GASSA |
| 211372 | VSKKK | 294737 | NGNQT | 335371 | SIAAA | 363868 | KESYV |
| 211373 | GLWSG | 294740 | SQSYR | 335376 | YLTGY | 363869 | GKSVC |
| 211377 | MNLEF | 294774 | ERVTS | 335382 | TIFTV | 363871 | PATVC |
| 211402 | FQKML | 294785 | GAVSY | 335384 | QQYED | 363872 | TVSLF |
| 211475 | AKALK | 294794 | VIFAY | 335385 | WIQHR | 363873 | TRCGG |
| 211936 | LHPGI | 294811 | PAVAL | 335392 | IHTHL | 363879 | RGRLR |
| 211998 | TPWYQ | 294816 | SYFTS | 335397 | VPHHL | 363880 | YSYVA |
| 212015 | PSNKS | 294818 | FPQLI | 335400 | HDVIS | 363888 | DKPPA |
| 212355 | SSSTA | 294829 | TFEAH | 335409 | QSRWR | 363891 | PPEES |
| 212369 | LELLN | 294848 | KRMEA | 335416 | LERFH | 363894 | FSPLK |
| 214869 | RPVPT | 294889 | VSFVR | 335424 | IYKMN | 363902 | PPRAS |
| 215057 | VHSAE | 294904 | FQPNK | 335434 | GGTVV | 363903 | EQVRS |
| 215061 | GSVYF | 294923 | RHTKL | 335437 | HTINR | 363906 | LSHSG |
| 215071 | LEMIV | 294947 | PVSIM | 335444 | PEDVD | 363907 | VLWEE |
| 215095 | GTLGL | 294952 | KNKSR | 335447 | KGAHL | 363911 | KNFNK |
| 215202 | VSANT | 294954 | RYTEC | 335463 | CHFYH | 363913 | KKPIH |
| 215368 | TLLGS | 294964 | VKASG | 335481 | RDERG | 363918 | LLLKF |
| 215375 | VKALE | 294973 | KKPLG | 335484 | GQRCS | 363921 | RPFYS |
| 215530 | PVLVS | 294993 | WLGST | 335486 | PSHGT | 363930 | QRGSA |
| 215531 | FQGQG | 294997 | CTTGF | 335493 | SGCHF | 363934 | IPMMQ |
| 215539 | HFAPC | 295006 | CFSVL | 335498 | RNLTA | 363939 | LVLIV |
| 215555 | EETPV | 295024 | SSMDS | 335500 | PAELK | 363942 | RQFNY |
| 215565 | PKVAL | 295025 | EFFQV | 335503 | SYGFY | 363944 | GLQLF |
| 215567 | IPFLL | 295030 | EKQDL | 335511 | CLWNT | 363952 | RIYIG |
| 215570 | ERANM | 295031 | TVLLC | 335525 | SSISS | 363965 | LSVLF |
| 215574 | GTEES | 295049 | QVTCM | 335528 | ARETS | 363970 | ETFGF |
| 215582 | SEEDD | 295050 | SEESL | 335529 | SKIHS | 363972 | RKQDE |
| 215587 | YRLVQ | 295052 | VYAGF | 335535 | IGILA | 363976 | PCSGD |
| 215591 | TYFFS | 295055 | LVMYN | 335536 | TVCFY | 363979 | QNPTE |
| 215631 | SLQER | 295057 | RECEV | 335538 | GQKKW | 363980 | HPSLL |
| 215659 | KETPL | 295062 | RHRMS | 335540 | RDVAP | 363983 | GWHIS |
| 215727 | NPSRS | 295066 | FEDRN | 335544 | TLGPG | 363985 | YSKHQ |
| 215730 | KVRQL | 295075 | FEGPN | 335549 | FLLNK | 363988 | ACYTY |
| 215739 | LGADI | 295079 | SKLLE | 335557 | YERGH | 363993 | KFYDE |
| 215742 | SSSMA | 295082 | LQSCK | 335565 | SRLAC | 363999 | ITWSY |
| 215743 | ANTFL | 295083 | AIKAE | 335566 | CYSSC | 364000 | PVCFV |
| 215754 | NSTFA | 295087 | RLKIR | 335567 | NLTKY | 364005 | MTEVT |
| 215770 | FIYFI | 295095 | GSEED | 335568 | YGSPW | 364013 | SSRMV |
| 215773 | VMTFL | 295101 | SDRFT | 335575 | TFFSW | 364016 | REANQ |
| 215780 | IARIP | 295108 | AIFHD | 335578 | DLGFL | 364023 | SSFLV |
| 215781 | GQLPR | 295110 | EHVFW | 335579 | HFLYW | 364028 | KCEVW |
| 215790 | EDTYL | 295112 | ILNFD | 335588 | ALEHF | 364032 | KLAQL |
| 215793 | GGRKK | 295113 | RQARN | 335592 | AASPK | 364033 | FLYFS |
| 215794 | MKMEC | 295119 | YMFGW | 335595 | ILVRS | 364035 | CPGKF |
| 215812 | ELTPV | 295124 | SSASP | 335596 | KNVMM | 364037 | LATVY |
| 215829 | FQKRR | 295131 | EGVQL | 335598 | LNLSI | 364041 | LVKKS |
| 215832 | PGYRS | 295133 | VEGGC | 335605 | TDAAV | 364042 | GNSET |
| 215838 | RLVSW | 295136 | STQTM | 335610 | LDSGH | 364048 | LSLTR |
| 215855 | RFPSS | 295137 | HRKCF | 335612 | PARTH | 364052 | LGSTC |
| 215861 | FHPSN | 295148 | VDGMA | 335614 | RTYSC | 364053 | LPQSC |
| 215872 | RAWNC | 295150 | ILVPE | 335615 | KKSAL | 364054 | LVKTS |
| 215882 | VWKTD | 295156 | CDIQK | 335618 | RPKSE | 364057 | PPPEK |
| 215885 | KSWSQ | 295171 | HRSVL | 335620 | KIFRF | 364070 | VITLL |
| 215886 | FKLKE | 295181 | MNRIQ | 335623 | AATQV | 364076 | HQGLL |
| 215906 | FAPGR | 295190 | DGAHV | 335628 | TGSEE | 364083 | ILEPA |
| 215909 | CVAFD | 295201 | LAGYQ | 335629 | HPMQS | 364084 | GRGGD |
| 215912 | GTPGA | 295206 | KDESE | 335632 | IRFLH | 364089 | GQPSV |
| 215917 | PSATD | 295208 | GKKTL | 335636 | PEDIE | 364092 | LSLNC |
| 215935 | EEDDE | 295210 | KQGID | 335642 | LEKWT | 364094 | FIRVP |
| 215938 | EPAGY | 295213 | GLPRF | 335644 | QEPTP | 364098 | ILMLV |
| 215939 | TEPPK | 295220 | YLILE | 335650 | SLSVS | 364107 | GDAQP |
| 215941 | SMEKR | 295225 | FENVI | 335651 | HVTFY | 364114 | TGLVS |
| 215956 | ERLLV | 295226 | RNFKK | 335655 | APGPH | 364115 | HVDPA |
| 215957 | PRDKS | 295228 | ECGCA | 335657 | YEYPS | 364118 | LKFNN |
| 215960 | LIITD | 295237 | QAEFS | 335660 | FTGFY | 364119 | RAKAN |
| 215980 | SLEDL | 295240 | WEVMS | 335670 | TIHMG | 364122 | LYHSP |
| 216014 | LPMPI | 295246 | VYFSF | 335675 | APLAE | 364125 | ENQGS |
| 216019 | PPSRK | 295251 | LNQWD | 335677 | KGVQN | 364126 | WSRPS |
| 216024 | GDAKE | 295256 | PQTKL | 335808 | FLEAP | 364127 | RRGPL |
| 216027 | KKIPL | 295266 | FKSVS | 336073 | ILDAL | 364130 | LTHQP |
| 216029 | SRKRK | 295268 | AADIM | 336127 | IPRVL | 364132 | SLSGW |
| 216034 | LPGKI | 295269 | LLQKK | 336417 | YTSSK | 364133 | EGIKM |
| 216036 | IEFKP | 295297 | EDDEL | 336522 | CLSDA | 364135 | PQPPK |
| 216038 | AVIKG | 295304 | NLNCI | 336524 | FTALK | 364138 | CQGCL |
| 216039 | WRTDV | 295314 | QQKKR | 336528 | GITGS | 364140 | TDARK |
| 216044 | PASGC | 295317 | GGPIS | 336547 | QILLM | 364141 | FFKGP |
| 216061 | LNGPV | 295321 | NKMPL | 336552 | AKSES | 364144 | IQSSD |
| 216064 | GEPAH | 295324 | MDKNK | 336565 | RPEVP | 364145 | VPAHY |
| 216068 | VWKCS | 295349 | LVTSL | 336571 | LTGYV | 364150 | KERML |
| 216071 | LKSKK | 295367 | KTKQK | 336587 | THSSK | 364152 | RRNIF |
| 216075 | GILSW | 295373 | LVTTQ | 336606 | QATGT | 364153 | SSRKL |
| 216080 | TRRKK | 295379 | ECGCR | 336607 | SELDQ | 364154 | SRRMF |
| 216085 | GPGPP | 295381 | PKTEA | 336616 | VWKPD | 364156 | LEEAD |
| 216099 | REILQ | 295382 | GEGTP | 336627 | ALNKI | 364158 | NSPCA |
| 216101 | RCSLM | 295408 | SEVLM | 336655 | DDWEF | 364163 | GEGKK |
| 216106 | IMPGL | 295440 | SGLGC | 336661 | MAFFP | 364166 | VTWAL |
| 216115 | HLLLK | 295448 | MKDGN | 336666 | EMGLA | 364171 | LQGLG |
| 216117 | GLYAM | 295450 | TATAP | 336673 | LQPQQ | 364177 | PHPYF |
| 216121 | ISPLQ | 295452 | GYLYL | 336687 | EDEAQ | 364178 | HIRSI |
| 216122 | LYRLK | 295453 | TATAP | 336693 | SKQWS | 364180 | PTHTC |
| 216127 | KCTIQ | 295454 | LYYVH | 336701 | PEEEL | 364188 | KEKSD |
| 216129 | VTCLV | 295461 | SRTDD | 336702 | KAMAK | 364190 | SDTSN |
| 216133 | RSGKF | 295463 | ASAAP | 336712 | AFYGV | 364192 | LELPT |
| 216139 | LTSTS | 295470 | NYQPY | 336719 | KKSIK | 364204 | WRAAL |
| 216144 | RSGMK | 295488 | STDKA | 336724 | NHYKK | 364211 | STGGP |
| 216160 | VVTAP | 295491 | IRYRE | 336725 | DWWGN | 364212 | LQKTQ |
| 216171 | QALLF | 295500 | HSPKT | 336727 | SNAVQ | 364219 | APPGV |
| 216177 | PTHQA | 295509 | VQIKT | 336729 | LLPHT | 364220 | NPNYV |
| 216178 | ADQEL | 295522 | GKDYV | 336733 | SWRTE | 364222 | PVSAA |
| 216180 | LEKSL | 295542 | TAYAG | 336740 | PEVPD | 364235 | WLLSI |
| 216181 | AKPAE | 295549 | RPSLC | 336741 | EYSQY | 364236 | HLKIH |
| 216185 | KKLIG | 295550 | SVMGT | 336747 | VTEKE | 364240 | GEGWP |
| 216187 | NKEEL | 295559 | SCFLT | 336750 | RMRKI | 364243 | PPAPP |
| 216190 | EEEET | 295568 | LQKVL | 336752 | ATPAE | 364246 | QTPGC |
| 216194 | AELCL | 295569 | TKVTR | 336753 | AENIV | 364249 | NILCS |
| 216198 | PGLGL | 295572 | PIELN | 336762 | IFRRP | 364252 | STPRC |
| 216200 | LVAES | 295588 | LKTEL | 336764 | VPRPA | 364254 | MHLLL |
| 216211 | SKLQD | 295589 | SVPPQ | 336775 | DGHWF | 364257 | PTPPC |
| 216214 | DAGGS | 295598 | KETYY | 336786 | VCVRN | 364260 | VSLGS |
| 216218 | FGGQA | 295600 | TEHTC | 336790 | KKRVP | 364261 | AALNI |
| 216223 | PTHLV | 295605 | KKTKL | 336799 | EGEEY | 364263 | ISSIA |
| 216225 | QKYGH | 295619 | CLAQK | 336800 | KHLPA | 364265 | FVSAM |
| 216237 | QETDD | 295622 | MLCQV | 336812 | LQLHF | 364270 | TYEKM |
| 216241 | EKGRL | 295628 | KVLLG | 336817 | EEKIL | 364271 | HKRMK |
| 216252 | GFKKR | 295633 | STKEI | 336829 | PEDDL | 364277 | TRDKT |
| 216259 | TAHEA | 295640 | APKGS | 336831 | LNRIK | 364281 | EMLAF |
| 216264 | PDSHS | 295641 | LALDR | 336832 | PFHGT | 364282 | PAPSL |
| 216268 | IYFQY | 295645 | SINSF | 336842 | AAFVW | 364287 | RKQQC |
| 216271 | PHLVA | 295658 | ETSTV | 336854 | KEIVA | 364289 | CALQA |
| 216274 | NHSGK | 295666 | EGAEL | 336856 | NRLDR | 364291 | DRALG |
| 216277 | LRLNR | 295682 | SKKRN | 336861 | SKPKK | 364293 | VRRKK |
| 216286 | PTGRK | 295683 | VSSNL | 336865 | CIRHR | 364295 | PLLPS |
| 216294 | KRRKH | 295685 | TFSSR | 336866 | LKVPA | 364298 | RKMFG |
| 216297 | KKKRK | 295694 | PRPWG | 336868 | EEGLG | 364300 | PPGLK |
| 216327 | CDSVD | 295704 | SKDGS | 336871 | DLSPH | 364304 | PGQSQ |
| 216330 | LVDID | 295709 | PAHSM | 336888 | KAAES | 364310 | ASQEY |
| 216336 | METPL | 295713 | KSCTV | 336894 | PEDSR | 364320 | SEDDD |
| 216338 | TMKRL | 295718 | KALPQ | 336914 | GAAAP | 364321 | QGCQV |
| 216361 | LESQQ | 295727 | GGHYH | 336923 | YVKLL | 364325 | CLPVN |
| 216366 | TTPIY | 295728 | RRVQH | 336931 | LKVQT | 364327 | RGERA |
| 216367 | KLQGF | 295731 | SGAGS | 336936 | RADIL | 364334 | KVLCL |
| 216373 | AETPQ | 295736 | AETSL | 336972 | PTEAS | 364346 | IQASE |
| 216392 | KVNGN | 295738 | FRSQK | 336984 | IPNRK | 364348 | QGLYV |
| 216407 | DYKPV | 295743 | FYTTP | 336987 | ICKIV | 364349 | YLNHP |
| 216410 | RRFLK | 295746 | VNLSI | 337008 | AEAAS | 364354 | NRAWR |
| 216416 | VLVSS | 295748 | CLYKN | 337014 | WLCIQ | 364356 | TTEWS |
| 216420 | KPKTL | 295749 | PAKQK | 337019 | FVINK | 364358 | ASQMS |
| 216442 | DLLFE | 295755 | CCHLT | 337022 | SPTSQ | 364359 | ALPCL |
| 216445 | FFDQE | 295756 | REPIN | 337023 | SHLFL | 364361 | GWRNF |
| 216446 | IKKLT | 295757 | PPAVP | 337034 | LLLYT | 364363 | NKTRK |
| 216452 | TSTSP | 295759 | ELSGH | 337040 | YSGTP | 364370 | PRPQL |
| 216453 | DLLSL | 295761 | TTFAL | 337044 | KPPNE | 364371 | SPCQA |
| 216455 | DDDNM | 295766 | LPTRW | 337053 | QQPPQ | 364376 | NSPRC |
| 216456 | FFRSH | 295767 | EEGSS | 337056 | MVAPL | 364380 | PPGFK |
| 216463 | LGQPR | 295770 | SKKTV | 337065 | RVYEE | 364382 | SVIHV |
| 216465 | TELRA | 295771 | MTVEK | 337088 | QRKGL | 364387 | ERRAG |
| 216468 | IYYTS | 295774 | DKTRQ | 337103 | QGHQP | 364389 | KTVLR |
| 216471 | PCTEP | 295777 | DFEEL | 337113 | RFRKS | 364391 | CKCKM |
| 216479 | GARLF | 295802 | QKKKN | 337122 | IEEDP | 364396 | PAGGG |
| 216484 | EETED | 295806 | HPAVP | 337127 | MACKK | 364397 | MNSPF |
| 216487 | EPNFL | 295809 | PDFFF | 337128 | QEGAP | 364398 | FPALS |
| 216489 | INPDS | 295822 | IKPCK | 337133 | NPTKS | 364403 | LNSVP |
| 216492 | ALRRG | 295824 | CKTLM | 337144 | GPHSS | 364404 | INRSC |
| 216513 | DMQDL | 295833 | FHKKQ | 337146 | GLKLT | 364405 | APLRQ |
| 216520 | QQAGK | 295834 | ISKRI | 337159 | IEHLF | 364407 | QVKMH |
| 216538 | VDSGN | 295839 | KTEEQ | 337181 | LQRTS | 364412 | NTKVR |
| 216540 | SPCTA | 295844 | DLLPP | 337196 | FIYYE | 364417 | NICCK |
| 216554 | DIDAI | 295851 | MHYSE | 337209 | IRLRK | 364418 | QQLFD |
| 216602 | ESRDQ | 295859 | NDESF | 337212 | KVSCP | 364429 | GEDGS |
| 216605 | VNGLF | 295862 | PMEQD | 337222 | VFVKV | 364430 | DARDL |
| 216612 | LLPDD | 295872 | STHVS | 337224 | LWMAG | 364431 | RQRLA |
| 216629 | LFWRN | 295874 | MESLV | 337226 | ILVET | 364438 | IKEDN |
| 216639 | KRVQK | 295878 | SLVNE | 337239 | KMPAK | 364444 | QGCQV |
| 216714 | LYLAL | 295884 | TDLSI | 337240 | DYRSI | 364448 | REEAE |
| 216727 | WYSPY | 295886 | PESSS | 337255 | TKPSP | 364449 | EQAGV |
| 216733 | TSLAP | 295887 | PTLKV | 337262 | QKYAM | 364464 | AEMLF |
| 216756 | GHRAL | 295888 | GPRNC | 337265 | GAAKH | 364467 | DCEVS |
| 216774 | GFNNM | 295890 | FISRK | 337266 | YEDVT | 364473 | KSRIW |
| 216775 | PSPSP | 295894 | FTNQI | 337281 | DGEDG | 364475 | KHSAQ |
| 216780 | RVHKM | 295896 | GPGDS | 337289 | KLDAS | 364476 | NTSSR |
| 216797 | QRLTL | 295898 | PLPSK | 337313 | KKASE | 364482 | RLPET |
| 216799 | GNGNA | 295899 | TDPKP | 337320 | KISVI | 364486 | NQAGS |
| 216807 | SIKHS | 295901 | RVINM | 337331 | GVGKK | 364493 | QCTEL |
| 216822 | AEWVS | 295902 | NCIIS | 337332 | KAVPS | 364494 | SAVAA |
| 216832 | KDKRR | 295903 | EVRVL | 337340 | PGKPS | 364501 | PQRRS |
| 216840 | SSVLT | 295907 | AQEPC | 337353 | MWVPS | 364502 | PPYAP |
| 216862 | AFCQR | 295908 | SGRWA | 337358 | GWALE | 364506 | PRPPP |
| 216911 | ASKQS | 295910 | INEEI | 337363 | GGSRL | 364512 | GKKSQ |
| 216923 | DSIPD | 295920 | TTEWE | 337383 | FEPSW | 364515 | IRTPK |
| 216951 | NPYPV | 295924 | NTVSI | 337386 | SISDD | 364519 | NDEDD |
| 216962 | NIPRD | 295926 | GRHRR | 337393 | EKPHH | 364520 | TMRRD |
| 216968 | GGRRC | 295927 | AQYVS | 337396 | KVKRI | 364522 | NSSWV |
| 217026 | TLILS | 295930 | GSPVA | 337432 | IHHVQ | 364524 | MEQFT |
| 217043 | KFTWF | 295934 | TNLLE | 337439 | LMTTV | 364543 | SLYTQ |
| 217073 | KAYMH | 295937 | CQLDD | 337443 | KPEPI | 364550 | PQQQL |
| 217086 | KIAVS | 295941 | FNRKS | 337445 | KVRAQ | 364552 | KRSLF |
| 217109 | RSTTD | 295943 | VEQVS | 337446 | LGARA | 364561 | FLSPI |
| 217121 | DPAPF | 295951 | ARASP | 337448 | PVTSG | 364566 | LSAPG |
| 217130 | KAPEK | 295956 | HVTVP | 337451 | GPVPV | 364567 | STEEK |
| 217131 | GDPIV | 295958 | APPVD | 337452 | NMKTD | 364578 | LVDGF |
| 217133 | EDKGH | 295959 | ELVLD | 337455 | TGPEG | 364580 | LLAQG |
| 217159 | LQSSV | 295962 | NKKLD | 337459 | EVFSS | 364582 | NPRTL |
| 217169 | RTFLS | 295966 | YYELV | 337461 | WIPVL | 364584 | YHYVA |
| 217173 | LRVHF | 295980 | LADLG | 337463 | PGGGG | 364591 | RGKTG |
| 217182 | QKAGK | 295981 | AGDGT | 337466 | CMSIL | 364592 | DLGFL |
| 217185 | YENPT | 295982 | ASSQR | 337475 | RKKQE | 364595 | MAPQQ |
| 217188 | HRCHP | 295984 | DTIEL | 337477 | STQEW | 364597 | WNMVP |
| 217233 | VVLYG | 295987 | SLFSD | 337478 | SAVVK | 364603 | GESPS |
| 217246 | RNGTK | 295989 | SMELS | 337494 | EIRHS | 364609 | WNMVP |
| 217254 | LHCPA | 295992 | KNKQC | 337500 | RIDFG | 364613 | DLGGL |
| 217256 | GQPPQ | 296003 | SSNAL | 337501 | QEPGQ | 364614 | LLWQT |
| 217260 | PGLQP | 296015 | NKSKN | 337510 | GRYVS | 364620 | WNMVP |
| 217270 | CIRLK | 296026 | KGSTN | 337512 | LQLQN | 364621 | AYALG |
| 217289 | TGGQD | 296028 | DESAD | 337513 | FQQAS | 364623 | HHLKT |
| 217299 | YETTV | 296029 | KLLES | 337518 | KHKRK | 364627 | LNYIC |
| 217305 | ELFDA | 296031 | SDKNN | 337541 | AFGGQ | 364629 | CGMGG |
| 217315 | AVKID | 296043 | LTSPL | 337542 | PTRRN | 364635 | WHMVP |
| 217320 | AWLLS | 296046 | LKHTS | 337548 | PYFED | 364638 | WLEEN |
| 217381 | LGLLA | 296051 | KKPLT | 337555 | EKVYH | 364639 | RSSRH |
| 217386 | TFSQR | 296059 | QPGII | 337560 | ARRRL | 364643 | LTVAS |
| 217398 | SRPSN | 296084 | LGAYV | 337561 | PSAIA | 364645 | QLSSI |
| 217402 | WAGSM | 296088 | CCHVI | 337572 | PQSEV | 364649 | KKASV |
| 217407 | QYMRV | 296091 | LHSGD | 337605 | AALPY | 364650 | GCEVS |
| 217420 | TNAED | 296096 | ALGDK | 337618 | NLTAF | 364660 | NFLPL |
| 217423 | RCQEA | 296097 | SEDDF | 337629 | NWELN | 364661 | ENDQS |
| 217426 | DHYRY | 296098 | RPDGP | 337632 | RFGIA | 364664 | SSSPD |
| 217428 | FCKFT | 296099 | DSVGK | 337641 | GIIPT | 364668 | YPELL |
| 217429 | YPSYQ | 296102 | KIRSL | 337649 | ISTPV | 364675 | NVLIR |
| 217446 | MLVLK | 296105 | PEATR | 337653 | HFAML | 364677 | NVLIR |
| 217455 | SESKL | 296111 | NHIRD | 337679 | PWLNS | 364683 | KLEFV |
| 217456 | SLQAV | 296121 | EDENE | 337688 | SHEKS | 364685 | SCGSC |
| 217515 | KGESH | 296125 | VLITK | 337701 | GALGL | 364691 | GPPEK |
| 217537 | LRKGT | 296126 | ESEEE | 337722 | QTVKT | 364694 | GNFGM |
| 217652 | KDKDD | 296127 | YQYVV | 337724 | SEKTS | 364699 | MLWKL |
| 217740 | HSNTT | 296129 | MEPAE | 337731 | PEWDL | 364700 | YENQE |
| 217800 | HIGHK | 296130 | QFGIV | 337732 | GLTPS | 364702 | QQNKY |
| 217893 | DYDNL | 296135 | YEPED | 337733 | IQANS | 364708 | GGTVA |
| 217901 | RAVEA | 296137 | GSDFL | 337736 | YYTSL | 364709 | SSPLK |
| 217939 | YIHVF | 296140 | LSAGF | 337757 | IANFF | 364713 | EQPQV |
| 217957 | GVVKA | 296142 | VKTAI | 337759 | TPSPK | 364721 | EQEEY |
| 217958 | RMVEG | 296143 | WRIQS | 337761 | GACGC | 364722 | EQPQV |
| 217961 | KRLSR | 296144 | FSLQL | 337797 | EPAQA | 364731 | RAPFP |
| 217964 | LDLRK | 296145 | IQSYY | 337804 | KVSPH | 364732 | KKRKK |
| 217971 | ARKND | 296161 | AKGIE | 337816 | GVASR | 364737 | AEENN |
| 217999 | YIVVD | 296181 | NGTVD | 337829 | SKRKR | 364740 | FKQRR |
| 218004 | DWSSS | 296202 | EGKTS | 337831 | SDGQC | 364742 | VRAKR |
| 218006 | VRNKP | 296204 | PREFA | 337838 | KRKAE | 364745 | EQPQV |
| 218008 | LNIET | 296214 | PRADY | 337839 | RKKKS | 364749 | ESAPE |
| 218054 | EKGHC | 296215 | EVSDS | 337843 | SRKGN | 364755 | GGTEA |
| 218056 | RREQK | 296218 | IAPKK | 337850 | ENAAS | 364756 | ASVQC |
| 218068 | GDGQP | 296220 | TQPAE | 337852 | ARGEL | 364758 | PRAHP |
| 218075 | DVHKK | 296223 | KKLAF | 337854 | GRPAL | 364762 | EQPQV |
| 218089 | GISTF | 296233 | VRSVN | 337862 | GSPHL | 364765 | EIYGY |
| 218099 | KTKLT | 296238 | PPSGV | 337889 | RPALS | 364770 | PALGK |
| 218104 | QGAST | 296252 | PELQL | 337907 | SWLHS | 364771 | YFGGT |
| 218147 | QSCNS | 296254 | SRKDT | 337915 | TVSGA | 364776 | CSVFL |
| 218172 | LRAAR | 296255 | ILDAL | 337926 | KRANK | 364781 | VGMLV |
| 218176 | KISSL | 296257 | HQQLL | 337946 | SCLLN | 364782 | VYGTL |
| 218224 | TKQQD | 296266 | DDPGP | 337949 | VMIHD | 364788 | YGCHA |
| 218230 | RLLPP | 296270 | TFHLN | 337969 | LTNLF | 364795 | CSVFL |
| 218249 | SCKIS | 296271 | QVAPA | 337972 | ERSGR | 364801 | IEEVD |
| 218316 | DEMAV | 296273 | LSQNC | 337991 | KLKRR | 364802 | IEEVD |
| 218340 | IQMGI | 296277 | TKLGL | 338004 | RRLPE | 364805 | IEEVD |
| 218343 | SSMQR | 296280 | PQVER | 338010 | GPQAS | 364811 | TTADF |
| 218348 | FMDVN | 296288 | KAKRQ | 338013 | KKKKK | 364813 | LQQKQ |
| 218364 | DDDDI | 296289 | LITKA | 338019 | RCLIL | 364814 | EVFYN |
| 218388 | RSQIA | 296292 | TDKMT | 338030 | RIEYA | 364815 | FQKML |
| 218432 | VEGRK | 296302 | NLENV | 338034 | VPEGL | 364822 | SLKLP |
| 218436 | LSYVL | 296318 | AVAPL | 338050 | GITDD | 364831 | PPAPK |
| 218439 | GFSYK | 296325 | RHNEL | 338062 | ALGNP | 364836 | FISLS |
| 218507 | CNNQY | 296326 | TPGAW | 338067 | GTTSS | 364840 | GLASW |
| 218516 | LKDLL | 296327 | LNLKA | 338072 | KDTSS | 364847 | FFWVE |
| 218548 | KNMYY | 296328 | VQERN | 338087 | KIVSR | 364858 | THKLS |
| 218652 | YFFLL | 296333 | GHFSL | 338089 | EFIDV | 364860 | VQLEA |
| 218721 | VKTMG | 296343 | VLEAT | 338093 | AEEAF | 364863 | WGRRG |
| 218758 | RRARP | 296350 | LPPAL | 338104 | IWAFE | 364864 | GLVPK |
| 218867 | NHICL | 296358 | VYCKI | 338127 | PGARS | 364869 | EETED |
| 219022 | LQKPQ | 296370 | KAGLK | 338141 | FKTVA | 364878 | LFPGN |
| 219054 | KARAQ | 296380 | VKKAK | 338146 | TSCPI | 364883 | RYKGE |
| 219066 | AAQGL | 296387 | GPLGV | 338157 | YLCYV | 364885 | PPPAE |
| 219069 | RTHTG | 296394 | NILFV | 338160 | PEGHV | 364893 | ISHQN |
| 219070 | DWLGC | 296411 | FMSQF | 338161 | VTVHK | 364894 | ATSIL |
| 219084 | SSNED | 296412 | TVVKI | 338164 | TIDVC | 364897 | LGPGQ |
| 219091 | PPAPT | 296414 | SFIFK | 338165 | DHRFS | 364898 | YDVVN |
| 219097 | KATAE | 296417 | QQKTV | 338185 | FDTPL | 364902 | LRDEF |
| 219150 | TVQAK | 296420 | GKTKN | 338186 | WDRLR | 364905 | RKISR |
| 219162 | CSCCP | 296422 | TLEHH | 338191 | MGLLV | 364909 | QFCVR |
| 219168 | HDEVS | 296424 | GGWSL | 338207 | ESQVQ | 364912 | VIASV |
| 219169 | LHNFG | 296425 | HNKQD | 338218 | NREMR | 364915 | IVFIN |
| 219172 | VFPAQ | 296435 | PRTES | 338225 | LFAPY | 364919 | KCVIM |
| 219197 | LVFPL | 296437 | TRSPS | 338228 | VERQT | 364921 | AAGCC |
| 219204 | NNLRH | 296438 | YSLNT | 338235 | ARRET | 364922 | HPLSG |
| 219207 | AGGYA | 296440 | KVTDL | 338258 | DEIRV | 364923 | FFKVQ |
| 219235 | NKLSQ | 296444 | PKAAL | 338260 | SLKAG | 364929 | RAYNR |
| 219240 | ADHRR | 296449 | DDDGF | 338266 | REEKT | 364934 | AKALK |
| 219244 | SLERS | 296452 | FSSFW | 338272 | EIGAF | 364938 | SSAPS |
| 219252 | VLTIN | 296456 | THLGS | 338276 | DTEIK | 364943 | TRPHS |
| 219255 | SGFSL | 296464 | EMEVD | 338288 | CGSKL | 364946 | LVQQF |
| 219271 | LQEWV | 296468 | GRIQE | 338290 | LSNME | 364956 | RDPWC |
| 219281 | SMPLK | 296472 | REEAS | 338293 | MGIRC | 364957 | YCSVL |
| 219299 | YLAGK | 296473 | TPLTY | 338297 | ASNRK | 364964 | YAVVV |
| 219301 | FCSSI | 296474 | PPRPT | 338323 | AFNKI | 364973 | CMRRT |
| 219302 | HWLYE | 296477 | EVTGK | 338340 | KRYAT | 364975 | FLNSV |
| 219313 | KKEKK | 296479 | TTSSL | 338343 | TSVCL | 364976 | KDQQK |
| 219315 | LELTS | 296483 | TLEST | 338345 | YEPEA | 364978 | LWLLH |
| 219320 | VDLLA | 296484 | QRATP | 338348 | KSLGL | 364981 | FTVLC |
| 219334 | GEEEA | 296486 | KRLKT | 338349 | REYYV | 364982 | PPLAS |
| 219343 | ERKTD | 296487 | ESSDH | 338352 | PSSVR | 364986 | KVTTV |
| 219345 | VLLGP | 296490 | PTIDD | 338360 | SAPSP | 364988 | WNLKA |
| 219368 | HLKTQ | 296496 | VYFLP | 338387 | LPKRR | 364994 | GLWSG |
| 219400 | EESEK | 296498 | SVTKL | 338389 | TNLKV | 364995 | VERKQ |
| 219406 | SKEEL | 296499 | LQKVR | 338397 | CSIEA | 365002 | MPWHL |
| 219431 | QDTQA | 296503 | DEDEE | 338403 | SCWQV | 365003 | LLANL |
| 219439 | LSCTA | 296504 | DSGVH | 338405 | LYKGG | 365005 | LERSH |
| 219454 | QKHFQ | 296506 | PCNNQ | 338408 | QGDQT | 365007 | WPELD |
| 219473 | RVDLL | 296508 | VKFSL | 338413 | RYTIR | 365008 | VTKDK |
| 219476 | FTEFV | 296509 | IPVND | 338418 | FEQSW | 365010 | FSLDN |
| 219478 | PGAKA | 296511 | CGEDD | 338437 | ILGKT | 365014 | QNFSV |
| 219481 | FSAKL | 296513 | EQFNM | 338461 | RCFVF | 365016 | TIVKE |
| 219535 | ISQAS | 296518 | ASGID | 338474 | TPWGI | 365019 | LVKTS |
| 219542 | EVALH | 296519 | PNHYL | 338477 | MGGYD | 365021 | GLYTP |
| 219548 | WVEDY | 296522 | QAKTQ | 338481 | EEGED | 365041 | SAAGG |
| 219551 | IPPPP | 296525 | FLQYR | 338487 | LKKAD | 365045 | HRKCF |
| 219593 | TVSNH | 296529 | AFSKI | 338496 | IHTKH | 365046 | VKTIR |
| 219596 | GQGPD | 296543 | LANEI | 338510 | PENKQ | 365048 | ALKLL |
| 219599 | WSSGK | 296545 | FINTS | 338523 | YFLMK | 365051 | GPNQG |
| 219611 | GPRPL | 296557 | WLEEQ | 338524 | STARA | 365057 | MNLEF |
| 219627 | VPEKD | 296564 | SQSSL | 338529 | AYLHQ | 365058 | SYPTV |
| 219638 | KWQYF | 296575 | TSYIV | 338532 | QIEKN | 365063 | NVTII |
| 219660 | ILKAR | 296577 | FFLDD | 338533 | FIKAR | 365074 | VIANN |
| 219689 | SQKPQ | 296581 | QKRRM | 338548 | PVSSD | 365076 | PGKGL |
| 219700 | AWYYM | 296582 | KSVDS | 338556 | KKKKK | 365080 | STNEE |
| 219746 | QVSIF | 296585 | TELSS | 338562 | SVGLN | 365081 | ELSNN |
| 219758 | FWRIM | 296589 | VRYVD | 338572 | PNGLL | 365090 | VIANN |
| 219782 | PQPTC | 296591 | CTEEE | 338573 | DGVGD | 365103 | STKAE |
| 219789 | RAKKK | 296595 | YLTVA | 338579 | YFVIK | 365105 | KKASS |
| 219794 | ESFRI | 296596 | KNKDN | 338606 | PLLLK | 365107 | SGFSL |
| 219797 | KLSKK | 296597 | KSHNQ | 338607 | VTKDQ | 365108 | ETDQR |
| 219833 | CQISC | 296600 | GALCL | 338611 | HLLEA | 365116 | EKMQL |
| 219837 | VKLED | 296603 | IFNDV | 338613 | LTANL | 365118 | VFSSG |
| 219865 | TPLYI | 296604 | SVACS | 338624 | ESPVN | 365130 | TQQES |
| 219891 | LTFYS | 296619 | ADTHL | 338627 | CNVTS | 365131 | FADDP |
| 219919 | LSVIM | 296626 | LQNNI | 338628 | KRKMQ | 365141 | VFSSG |
| 220003 | HELHL | 296632 | LRKAL | 338629 | GDKQL | 365145 | MFFFE |
| 220058 | QQCIE | 296636 | VEDHA | 338641 | TNEAH | 365147 | QSTFK |
| 220062 | GFKIF | 296641 | AYLTK | 338642 | LKILQ | 365151 | LTNGA |
| 220166 | PIPLV | 296642 | VHIST | 338648 | PLNEV | 365152 | MALGV |
| 220188 | AGKHG | 296657 | WKKRT | 338670 | YMVGN | 365154 | EQAPD |
| 220244 | KKKKL | 296658 | LNKYM | 338671 | SPAVA | 365155 | PRHKR |
| 220325 | LPKAD | 296662 | QQSTG | 338673 | KRCKP | 365159 | ESFNI |
| 220420 | VDFAL | 296674 | ERPRS | 338690 | LDCAC | 365167 | SSIAH |
| 220478 | IYSSL | 296677 | VKTSY | 338692 | AQGVV | 365169 | PKINK |
| 220496 | EGPPT | 296679 | LYLAV | 338696 | IQIHS | 365172 | TKDEL |
| 220507 | FFCFV | 296682 | CREDE | 338703 | RILDH | 365173 | KTKGQ |
| 220509 | QLSWL | 296684 | RVSTK | 338706 | GNWGH | 365175 | LPPPR |
| 220514 | SKPEN | 296694 | LSHLV | 338707 | FERTR | 365193 | SRSQL |
| 220531 | PAKRM | 296695 | KSGPC | 338711 | CPPSS | 365198 | EVTNV |
| 220562 | CFKFI | 296702 | RRSTK | 338723 | PQQQG | 365200 | DKKCH |
| 220584 | QTGEH | 296721 | EMKKT | 338727 | LLAQQ | 365220 | DYCVF |
| 220592 | TMYFA | 296734 | KKEDL | 338728 | EQQSS | 365221 | ERETP |
| 220597 | DITRL | 296736 | FLQTK | 338742 | FRSEL | 365227 | ISELP |
| 220616 | KTYSK | 296741 | LFLLA | 338769 | QLLQD | 365243 | KQWKK |
| 220659 | VPNPP | 296754 | ADATG | 338770 | SQVYM | 365247 | GADMW |
| 220669 | ESYLE | 296755 | CKIEL | 338774 | DIKTE | 365252 | TLPFL |
| 220676 | EQEDL | 296775 | WSAVT | 338777 | LSQFV | 365255 | KNYMI |
| 220751 | QNKSM | 296776 | YSLPR | 338783 | WASPD | 365266 | FHLCS |
| 220764 | KTKGS | 296777 | LLKCL | 338785 | PETKI | 365270 | LWSSQ |
| 220772 | QSVFK | 296783 | AVLKR | 338788 | LDLEE | 365272 | SKEKL |
| 220809 | DNMRP | 296785 | QNIKE | 338796 | RENSW | 365280 | RILCR |
| 220812 | KIEKL | 296786 | KKQGQ | 338797 | HSGSL | 365285 | AVMVG |
| 220822 | RPNYF | 296792 | KKIES | 338799 | GYMPQ | 365289 | FHLCS |
| 220849 | DSGFY | 296794 | DCCAS | 338802 | AAGAV | 365290 | GIIAL |
| 220853 | QITQS | 296795 | KNSVH | 338807 | NELDQ | 365294 | RGRPA |
| 220876 | VELSG | 296799 | NIVYL | 338812 | KAEDD | 365295 | GAHRD |
| 220913 | DEADS | 296802 | AVLLV | 338814 | DEWEY | 365297 | VLEHE |
| 220940 | SNILP | 296803 | TSIPL | 338823 | CRRGS | 365298 | HSQDP |
| 220959 | NFGFV | 296805 | RSGLT | 338838 | FYDLL | 365301 | KSKTT |
| 221086 | MQESP | 296824 | AILEK | 338860 | PWPLG | 365302 | LHLCC |
| 221114 | TPVKN | 296839 | ETLLA | 338862 | SGKTK | 365303 | GAFAL |
| 221130 | LVTLR | 296847 | SNEAF | 338885 | PSPQL | 365318 | AEALK |
| 221132 | AVSLE | 296849 | HFHPS | 338886 | LPMNE | 365320 | GQSQP |
| 221138 | PDYFL | 296850 | QRTVL | 338887 | FANGI | 365322 | DQYCC |
| 221166 | VTQSD | 296859 | QVSAV | 338922 | KDIGQ | 365338 | DQCCC |
| 221167 | DLDFE | 296861 | LPDLL | 338927 | LFSPY | 365339 | KQSQC |
| 221169 | AKKKD | 296862 | HEDVL | 338934 | EFEAL | 365342 | KQSQC |
| 221200 | SQSVR | 296869 | YSISM | 338964 | GATIL | 365343 | WGRPA |
| 221204 | IPNVI | 296870 | SLAIF | 338967 | PETLV | 365344 | IEQTR |
| 221232 | DRDLQ | 296871 | EPVQE | 338984 | GGGVL | 365349 | DQYCC |
| 221233 | RYSKS | 296875 | KCTCR | 338988 | ESAVS | 365351 | GSEYP |
| 221249 | SATDA | 296882 | QVLSV | 338990 | DTLAL | 365352 | GAIPG |
| 221265 | DSDSD | 296888 | KSRSR | 339001 | AEEEA | 365353 | DQCCC |
| 221270 | CKAKV | 296921 | VTSLV | 339004 | NPLIH | 365358 | WGRPA |
| 221282 | KPFEV | 296930 | WRKSL | 339007 | VNRNV | 365362 | LGGGA |
| 221283 | DIALP | 296946 | SPPSM | 339013 | KIGII | 365369 | PTPGP |
| 221315 | NGNKP | 296951 | ANFSK | 339016 | HVCLD | 365370 | GQDDS |
| 221327 | LYECN | 296953 | PTSKV | 339027 | FGLFD | 365378 | WRKAI |
| 221347 | SPCYG | 296977 | QHPLT | 339030 | DSAGP | 365382 | KSHLL |
| 221355 | KFILG | 296978 | SETRF | 339046 | HTSRG | 365388 | LTRDK |
| 221363 | KEVDG | 296980 | CVEIP | 339047 | SSFVF | 365390 | EVPYP |
| 221399 | LSSFN | 296989 | RAKGR | 339048 | SKKAE | 365392 | STEGT |
| 221403 | PQDKR | 297001 | ETIDY | 339051 | VKKTF | 365395 | YIKGI |
| 221409 | GITGL | 297012 | KAQSK | 339052 | HHGMP | 365397 | FKGRT |
| 221413 | TMDTS | 297020 | ARKKV | 339057 | VGQQS | 365398 | RCCLL |
| 221418 | TFSKL | 297027 | RRKNR | 339064 | SEENE | 365400 | CEQAV |
| 221419 | AQHAS | 297029 | DSSKW | 339067 | NFWNG | 365401 | RVREA |
| 221421 | GLLFL | 297056 | SVDVA | 339068 | IVSLI | 365402 | VSLTA |
| 221431 | QPAVS | 297071 | SPRRY | 339071 | GPGGS | 365403 | GAEVL |
| 221444 | LVTEV | 297078 | DDRHK | 339075 | EHSAL | 365404 | QRVHT |
| 221448 | EAAPE | 297107 | KFNRN | 339087 | RINPK | 365407 | ITEEP |
| 221452 | GPEAT | 297109 | GKQLE | 339095 | PEFQL | 365411 | RGNVS |
| 221459 | LESRG | 297130 | ESEEL | 339109 | QPMVM | 365413 | ISNTF |
| 221461 | PPPSD | 297135 | MSALQ | 339115 | FNFGE | 365414 | QVSSL |
| 221462 | GQETL | 297142 | AVFHN | 339122 | IRWKN | 365415 | GARTW |
| 221466 | IPATA | 297145 | ILLLS | 339128 | SHSLG | 365416 | SSSTL |
| 221476 | IPAQK | 297146 | PYCVI | 339131 | LNRCV | 365418 | SWFSD |
| 221480 | EATTP | 297150 | QQKSL | 339135 | NRVSI | 365421 | FDQVT |
| 221482 | GVKPR | 297151 | SFLGQ | 339136 | GCSVA | 365423 | PPKPM |
| 221485 | PVRDY | 297156 | GSEVP | 339145 | AMDFF | 365431 | AGIQT |
| 221486 | SATSL | 297157 | GSDSE | 339147 | SHRLG | 365435 | GMKPS |
| 221494 | PPPTN | 297158 | VMPLW | 339151 | LEQAS | 365439 | DVEGF |
| 221496 | ECGCR | 297161 | LCPQR | 339153 | RSKLQ | 365441 | CVHAL |
| 221498 | PSRQL | 297163 | EHAQT | 339157 | HIFFK | 365443 | EATSQ |
| 221515 | CRVQP | 297164 | GAGSM | 339161 | TLHKE | 365448 | EAHRF |
| 221538 | EETDD | 297183 | GRDTR | 339168 | DKENM | 365451 | VICRE |
| 221543 | EGADS | 297185 | KEEKQ | 339174 | CCACV | 365460 | VKVLK |
| 221554 | DSESE | 297186 | KKKKK | 339175 | NLDLQ | 365463 | LVELE |
| 221561 | GEKSD | 297195 | ILAGL | 339179 | LQITT | 365465 | LLNSP |
| 221566 | DDQQE | 297203 | APSRF | 339182 | NEEQL | 365466 | LSSLI |
| 221567 | SYMPS | 297205 | ICSQL | 339184 | APSQD | 365469 | QSLGR |
| 221573 | ATPAR | 297235 | KSRGM | 339186 | APMTR | 365473 | RNSGP |
| 221576 | SKTPS | 297239 | TLVLH | 339191 | LQKEI | 365476 | IPSPR |
| 221671 | VNKEL | 297258 | YEKVE | 339196 | RRRLS | 365477 | KAKAS |
| 221730 | QDLLD | 297261 | AVKSS | 339207 | DLSPK | 365486 | RWWPF |
| 221735 | TGEMQ | 297265 | AAWAT | 339208 | ALKVI | 365488 | QHIKN |
| 221740 | RLYLQ | 297267 | IPGKW | 339216 | GDKNL | 365491 | STSAV |
| 221742 | NESAM | 297273 | DKSKH | 339238 | PLKCY | 365492 | NITNR |
| 221770 | GTIDL | 297283 | QGKAK | 339245 | GRYRR | 365494 | NCIVA |
| 221784 | EDDDY | 297289 | REEKY | 339250 | KPLQP | 365498 | LVHTF |
| 221797 | VCVCN | 297290 | GATFA | 339251 | TPDAQ | 365503 | NEVTV |
| 221801 | PKVKN | 297293 | DGEKD | 339255 | YEIHC | 365505 | ADGPV |
| 221804 | SYLKR | 297307 | GRVEE | 339256 | TTRFT | 365514 | NEWYV |
| 221818 | FQGRE | 297313 | KSIEA | 339260 | LESHC | 365522 | RCIYY |
| 221847 | MSLGK | 297316 | NYPDV | 339276 | QFYPL | 365528 | HKLEV |
| 221852 | VTQTP | 297323 | AGKEA | 339280 | QKSEL | 365529 | DSFKV |
| 221855 | GLDEI | 297324 | VNDNV | 339292 | LNDYV | 365530 | PNQKS |
| 221856 | NTSLT | 297325 | PGKHI | 339299 | LLPRV | 365534 | QNQFI |
| 221859 | HRWTE | 297338 | RFHII | 339307 | GLMKN | 365543 | DDDDF |
| 221891 | LEERP | 297345 | EDPCD | 339314 | SGEKL | 365550 | SDQCR |
| 221899 | DRMFF | 297347 | LAMRN | 339324 | RLKKD | 365554 | MEHLK |
| 221922 | PFESV | 297350 | KISCL | 339328 | TLLWT | 365562 | QPSWG |
| 221930 | SCKCS | 297354 | SFIFI | 339340 | LIRYR | 365567 | VVKVD |
| 221943 | SPVPA | 297375 | KSDSE | 339344 | RFTCG | 365569 | PLRTA |
| 221954 | ADVVS | 297404 | DDFWF | 339353 | VTAHF | 365570 | QEAGS |
| 221957 | PEEKK | 297405 | VCTMV | 339355 | LTPFF | 365576 | GSQPS |
| 221972 | QLEKP | 297416 | IRTLP | 339356 | AKGTQ | 365577 | GGCFY |
| 221973 | LSTPR | 297423 | ASAEH | 339366 | LQCIP | 365581 | AQKDL |
| 221975 | ANKKH | 297431 | LYDFL | 339377 | HCLHM | 365582 | TGAGK |
| 221978 | GYETL | 297435 | CTRVD | 339381 | HATFV | 365583 | AQKDL |
| 221980 | QLTSA | 297436 | RFCCL | 339382 | LAQQR | 365589 | MYGNE |
| 221992 | GVALI | 297438 | GDGIA | 339384 | NYLKG | 365596 | SSNGK |
| 221996 | KFQIL | 297439 | AKCCK | 339388 | NTTII | 365601 | TLKMK |
| 222002 | LFPWE | 297440 | VPATQ | 339389 | LPRLL | 365604 | FHPCL |
| 222005 | KDVSV | 297445 | GEPVK | 339398 | HQGPL | 365625 | REELG |
| 222008 | QMEPV | 297450 | RPLDF | 339399 | MILLL | 365630 | NCCNG |
| 222032 | LKIRE | 297459 | LAVQS | 339404 | LPGKS | 365637 | AKETS |
| 222033 | EAPGQ | 297468 | QQVLA | 339423 | KRKTR | 365641 | CGTPL |
| 222107 | IKTSK | 297477 | QWSPG | 339428 | YKFQV | 365642 | DITCP |
| 222115 | YRVQV | 297494 | DTNSP | 339432 | RWKFC | 365644 | AKGCL |
| 222120 | SSCSC | 297504 | HQHKS | 339435 | QSVSV | 365656 | LCSKP |
| 222122 | QHGAL | 297508 | SSPSQ | 339450 | PWLNS | 365662 | FITRA |
| 222124 | EIRSL | 297512 | LGAPP | 339458 | ANIMP | 365678 | QNACS |
| 222139 | YVACS | 297518 | DFCPP | 339461 | GKYVD | 365679 | DLRIR |
| 222145 | VATSP | 297524 | DVSEE | 339467 | SCILL | 365683 | SLSVK |
| 222157 | PSYAS | 297532 | GPEGG | 339469 | ELKCR | 365684 | AETVD |
| 222190 | MTPPW | 297533 | AMYRP | 339479 | EEEEE | 365686 | SQKQM |
| 222212 | STSPC | 297534 | WMLHS | 339484 | KLPKI | 365687 | FEFVS |
| 222214 | FTASK | 297537 | QGARP | 339493 | HLASE | 365692 | WSLLP |
| 222219 | RAYKI | 297540 | DLDIF | 339495 | APAPL | 365694 | GPPLP |
| 222224 | PHLTH | 297542 | GIKFQ | 339498 | WECHH | 365710 | YEVIY |
| 222246 | PNKTC | 297562 | ILVPL | 339499 | GSAQS | 365711 | LYRFI |
| 222247 | PNTFF | 297564 | FQSVK | 339503 | EVDTV | 365714 | RRRPH |
| 222248 | QETNL | 297565 | PRQDF | 339511 | SRKND | 365718 | CQYKI |
| 222249 | PSDCG | 297574 | KNCVN | 339522 | PKSSS | 365725 | WSELV |
| 222250 | RCMTC | 297578 | REKRK | 339527 | SWVPG | 365729 | SRPRE |
| 222254 | PPAAR | 297579 | VAVVK | 339529 | IAIEV | 365734 | KLGYF |
| 222256 | QDCAC | 297581 | SADKS | 339532 | WQCKE | 365744 | NGPPL |
| 222266 | PLGTP | 297591 | RSFTR | 339557 | KSASS | 365745 | VPECP |
| 222270 | RRFLN | 297592 | EYNSL | 339561 | QTCCF | 365757 | GHILT |
| 222271 | QLRQA | 297596 | DLSVL | 339564 | KEVLD | 365760 | QEWVS |
| 222284 | VNVHS | 297613 | RDTRS | 339565 | CMEDK | 365762 | ETRWS |
| 222286 | FSRDK | 297623 | PISEP | 339566 | APKKK | 365764 | PQLGH |
| 222301 | FFLFF | 297625 | FTWAS | 339577 | CRIQQ | 365766 | YQQYH |
| 222304 | MCCKT | 297632 | LETKS | 339578 | LLARK | 365767 | ETEAP |
| 222305 | EGTRQ | 297661 | RAGAS | 339580 | WASNS | 365769 | AKGLA |
| 222307 | EMTGE | 297668 | KKEEC | 339585 | FRGLI | 365771 | ESLVY |
| 222308 | IAARN | 297679 | TGSAQ | 339587 | GREHS | 365772 | DLGRS |
| 222329 | EHRDS | 297689 | ASDSG | 339605 | INLLG | 365773 | EMHQL |
| 222330 | LTNSS | 297737 | NLKNK | 339613 | WKITY | 365776 | TKPAL |
| 222339 | VQISG | 297761 | NPCCP | 339621 | GFLSQ | 365778 | AGNRT |
| 222345 | EKKEL | 297767 | TAKDK | 339624 | FSRHG | 365788 | QPLNN |
| 222374 | EEFFI | 297770 | LKKCP | 339626 | PSKGL | 365793 | QCLPL |
| 222379 | SRLLA | 297777 | VYSGL | 339627 | LGSYY | 365794 | CPLSD |
| 222381 | LYCEL | 297784 | AAGRQ | 339637 | EADCY | 365799 | PSILP |
| 222382 | ITSGP | 297785 | SQKNS | 339642 | LKIHT | 365800 | VSPGA |
| 222388 | SLPRP | 297788 | AETSS | 339659 | YRYLQ | 365802 | PVLVA |
| 222390 | KVPQS | 297801 | DGSRL | 339672 | RTHTV | 365805 | EEGCR |
| 222396 | PGSSR | 297814 | RDLKT | 339680 | KTSHP | 365806 | GEDEC |
| 222402 | QKYAM | 297819 | KFSKF | 339681 | VIPPK | 365807 | EFNLV |
| 222462 | VHTCK | 297820 | YNMKC | 339704 | EDILY | 365812 | ASKRQ |
| 222481 | RDHPY | 297830 | FMLTH | 339712 | LLKDS | 365815 | ESHSL |
| 222482 | RDNLY | 297848 | WGPGV | 339720 | ERPDF | 365827 | GSSNQ |
| 222511 | KMDII | 297857 | SKSDD | 339722 | LASAV | 365830 | SNVFG |
| 222543 | RKKQF | 297866 | IVTYI | 339723 | VTQRE | 365834 | PSPSL |
| 222553 | EAAHH | 297873 | PRLRK | 339726 | YFLQK | 365836 | GSSNQ |
| 222567 | KSNFL | 297875 | GKCRL | 339729 | SLWEY | 365837 | DGDTD |
| 222572 | LYCEL | 297894 | LWGVG | 339730 | DRFDN | 365838 | QSRER |
| 222573 | FRCNF | 297904 | SRKNP | 339731 | RPAVR | 365839 | PHAAS |
| 222574 | GSQQH | 297905 | LASAV | 339732 | LGPLR | 365840 | KSPAS |
| 222584 | NMEEF | 297908 | KELLG | 339737 | AISGC | 365841 | PTDWE |
| 222597 | RPYYQ | 297913 | TFFFR | 339740 | LWQNI | 365851 | ATSSG |
| 222598 | SGTLY | 297926 | RKDYV | 339743 | GGTVA | 365856 | RSLWY |
| 222607 | SFLQL | 297954 | DPVRS | 339750 | PREDL | 365857 | AHKGS |
| 222644 | ISWVY | 297977 | VSESL | 339754 | RSCEV | 365858 | APLSS |
| 222673 | FKNFS | 297988 | ALEHF | 339764 | MTSHN | 365861 | PQTAC |
| 222674 | LKQEK | 297990 | ERWTV | 339767 | CIDTP | 365864 | SPHHL |
| 222693 | QLSQD | 297991 | HKEQI | 339769 | PRLGS | 365865 | KGKWN |
| 222718 | QHLNY | 297994 | RNSVE | 339775 | KKKKM | 365866 | TCVVQ |
| 222725 | RTAIF | 298047 | HQTQV | 339787 | MYGGK | 365869 | SSEMT |
| 222726 | GAFRP | 298048 | SSCKV | 339793 | ILGPL | 365877 | RKYLF |
| 222728 | AKAGE | 298050 | QNRHI | 339800 | ANARL | 365883 | LNKGL |
| 222747 | EMEEL | 298068 | GFGLE | 339801 | QLLMP | 365884 | LTLKG |
| 222753 | LKTTS | 298085 | YVHSV | 339802 | QLVNF | 365889 | ATPAW |
| 222761 | VPLTR | 298097 | FSEDI | 339804 | LLKQQ | 365891 | DEWDD |
| 222792 | EDVLF | 298105 | NRRSV | 339813 | HLGSS | 365892 | FSLSS |
| 222800 | RGFLV | 298110 | SATFP | 339820 | FRGIQ | 365895 | QVVRW |
| 222803 | KHDEL | 298119 | RLELI | 339822 | RAGMA | 365898 | VLVHS |
| 222812 | GGIFA | 298124 | RLTSS | 339823 | VLCIM | 365899 | KVGAT |
| 222823 | RIICF | 298125 | TPVVS | 339826 | PPRGR | 365903 | LQRGK |
| 222902 | NQTTC | 298129 | MTSHS | 339830 | CKTVI | 365906 | RRIHR |
| 222956 | SGRRF | 298130 | FEIVQ | 339834 | RKQKI | 365910 | LKEKP |
| 222968 | LSLNK | 298139 | GGLFS | 339835 | RGERA | 365912 | AHNSI |
| 222969 | RGCSG | 298159 | EGKPL | 339836 | WKAEL | 365914 | PPQKL |
| 222982 | TLSGE | 298165 | KLHST | 339838 | LAQKN | 365918 | EVPSS |
| 222990 | GLCPH | 298171 | MQTVL | 339845 | EDIKK | 365920 | ETLKH |
| 223023 | DEWED | 298173 | GDAEW | 339850 | ITSLS | 365929 | EHPLL |
| 223026 | RSIQL | 298181 | EEDDE | 339851 | FPPGF | 365931 | VTKNL |
| 223029 | LKLLK | 298189 | QHCSQ | 339861 | QHASL | 365934 | YCSGN |
| 223051 | IDNNF | 298198 | IWRSV | 339867 | DELIL | 365935 | SKGAW |
| 223054 | VFLRT | 298215 | VASNV | 339869 | KQCIS | 365944 | VVQIF |
| 223061 | AASQD | 298223 | LWLLG | 339881 | ETVQP | 365945 | YSYYQ |
| 223073 | KWFDS | 298229 | LQLSK | 339889 | LENLE | 365946 | DNYDN |
| 223076 | AEQQA | 298231 | KTNLF | 339911 | SQLLS | 365959 | PSAEP |
| 223084 | EGPEV | 298248 | QVQPQ | 339912 | DHSHS | 365962 | ATAAS |
| 223095 | QVMEP | 298251 | VEISA | 339915 | VCFMG | 365963 | GPMQH |
| 223114 | CLTFI | 298281 | TVESV | 339916 | TPQKK | 365967 | LIKST |
| 223122 | KLGNP | 298282 | SDSLE | 339917 | DNDIM | 365970 | PSTSD |
| 223127 | SFVDP | 298283 | RVLHS | 339918 | QNGGW | 365988 | TRRFL |
| 223129 | IVQHD | 298288 | SDMLK | 339928 | RAERE | 365991 | KFHFN |
| 223136 | SKSKS | 298292 | LYDLD | 339952 | KSADL | 365993 | LLWMT |
| 223140 | SWGTR | 298295 | VIHEL | 339958 | VELTG | 365996 | TREPL |
| 223145 | NYVII | 298296 | NFCPE | 339959 | GKEHS | 366001 | TACKV |
| 223167 | GEEKD | 298298 | PCSVI | 339960 | ARGSV | 366006 | SLPKI |
| 223208 | GKPWK | 298299 | VAGLR | 339971 | GRSSA | 366007 | LQVTA |
| 223210 | APGMS | 298307 | NTSGS | 339973 | GVATS | 366008 | HLPCH |
| 223214 | NPKRV | 298310 | NVKRK | 339992 | RTLVM | 366015 | EDFKP |
| 223215 | FINSF | 298315 | VHTPH | 340009 | QGKPF | 366022 | NNANV |
| 223271 | ALPRS | 298316 | SNYKS | 340010 | DYLFS | 366024 | KKSSD |
| 223273 | HLKQL | 298317 | CLGAQ | 340017 | ALSSA | 366030 | VRKPQ |
| 223274 | LKTEL | 298350 | IHMGD | 340019 | GGGMF | 366031 | VLRCH |
| 223293 | KTPRL | 298351 | QNQYH | 340059 | VESPD | 366032 | RATQL |
| 223321 | LAAIA | 298352 | GWDGE | 340083 | SILGF | 366035 | NQENE |
| 223324 | WFTQN | 298375 | FHIEY | 340086 | STYGL | 366036 | KLSNL |
| 223336 | EVKKE | 298386 | MKPVS | 340087 | ILYAS | 366038 | ALDWW |
| 223357 | NFGDF | 298396 | EEDDE | 340088 | PRPGL | 366042 | CCCCF |
| 223361 | LGLGP | 298406 | KLWLR | 340089 | VATIS | 366043 | NFLYF |
| 223364 | DEKEE | 298440 | YSNNP | 340093 | DDENF | 366050 | VDMES |
| 223366 | CMLGQ | 298466 | AGPPA | 340095 | TLKSK | 366054 | RGLGL |
| 223368 | TASES | 298468 | DQNKG | 340100 | TEEEG | 366058 | KNNHL |
| 223369 | CCAIM | 298472 | ESESD | 340125 | DKIAI | 366061 | AQIQW |
| 223398 | QEDKH | 298492 | QTSQI | 340132 | IHTNK | 366062 | SEALP |
| 223428 | EGNPY | 298510 | QKVNQ | 340148 | KKKRK | 366073 | RMSGH |
| 223500 | QIPAS | 298527 | VDQLP | 340162 | FVISP | 366080 | ILKHK |
| 223528 | VIQLY | 298532 | RRRLV | 340166 | KKVEK | 366081 | EEDDE |
| 223608 | PSSCA | 298542 | NYFLA | 340176 | RELPC | 366083 | EEDDE |
| 223641 | KYTRS | 298545 | PKKIP | 340177 | GRGQA | 366084 | FETAL |
| 223642 | FLNGC | 298552 | HHEHS | 340191 | ELQAM | 366089 | PPLCL |
| 223795 | YSNSD | 298556 | AKYKA | 340200 | NCVPL | 366098 | FHLVR |
| 223836 | LDALK | 298557 | VHTAP | 340206 | NFKNS | 366107 | EEDDE |
| 223862 | LAKYC | 298569 | TPTGF | 340210 | WSLHP | 366112 | FQNQN |
| 223864 | FFIDK | 298585 | LEPER | 340211 | ENGDA | 366113 | ILFSP |
| 224024 | DRYSG | 298596 | PVINV | 340212 | SGLWW | 366116 | SLKFS |
| 224073 | GPRAK | 298622 | ELVEK | 340220 | FRPIR | 366118 | EEDDE |
| 224112 | FALAE | 298630 | SAGSG | 340228 | CLHVF | 366119 | VPPSL |
| 224140 | KRKLL | 298635 | RVERV | 340236 | CGKAF | 366121 | IFPPH |
| 224167 | CMTEQ | 298642 | KAMPS | 340237 | DDFVI | 366124 | TCQDA |
| 224181 | DEVRR | 298681 | IALWA | 340247 | AYRVA | 366134 | GKVET |
| 224237 | HDDLE | 298687 | MEVSC | 340261 | LALRR | 366135 | NWPQR |
| 224337 | AVKVS | 298690 | LDRVL | 340271 | KHRAH | 366140 | EDKQQ |
| 224356 | FHGEI | 298694 | PTTPL | 340274 | TCVVQ | 366144 | YAAAL |
| 224600 | LQDHL | 298699 | RNGSK | 340278 | LVLKD | 366146 | GLTQS |
| 224605 | TCACR | 298700 | LYFSQ | 340279 | KDDLP | 366155 | KIQRI |
| 224652 | LLFRN | 298705 | TSCPK | 340281 | EEEAD | 366156 | LNDVS |
| 224721 | EITEL | 298708 | GVSPC | 340292 | GDEEI | 366157 | SRPHF |
| 224756 | YRGPQ | 298717 | KPKNL | 340296 | LGQRL | 366169 | PAHPQ |
| 224764 | QDVKI | 298728 | AADPL | 340298 | ASPFC | 366170 | RDKGK |
| 224784 | HRKCF | 298738 | SKLWF | 340299 | RPPFS | 366171 | SEGFP |
| 224807 | YNKGL | 298743 | LGPLF | 340306 | GSTPN | 366173 | TAAAR |
| 224950 | YYTAF | 298746 | VIKTC | 340310 | YRKAR | 366174 | PKASF |
| 225171 | RNYEI | 298771 | SLYPC | 340312 | NSLAE | 366175 | PKASF |
| 225174 | CGQLS | 298777 | DFLNY | 340314 | DHSSL | 366178 | LEGFH |
| 225235 | PALKS | 298786 | ANARL | 340328 | GLQMD | 366179 | KQAKS |
| 225245 | LELSA | 298818 | FSIKL | 340329 | TQQVN | 366184 | GPQKP |
| 225276 | QLYQR | 298820 | TCQWN | 340330 | KRGKW | 366186 | SGGHS |
| 225296 | RTARN | 298832 | AHTKI | 340361 | KESSI | 366190 | EDYDS |
| 225298 | HYSDF | 298834 | EPRNR | 340363 | EWLLK | 366191 | LLVVD |
| 225371 | AWRGT | 298838 | ECVGA | 340369 | GTSDN | 366197 | HLLYK |
| 225387 | RRIQQ | 298841 | DPTKP | 340372 | PSLRH | 366201 | KEKGK |
| 225388 | DSPDQ | 298852 | LQYYA | 340381 | RVSPS | 366205 | AKYVL |
| 225396 | YITKG | 298854 | KPGFV | 340392 | QPSPG | 366207 | ERPSR |
| 225402 | EGHGD | 298875 | QYAIV | 340396 | PCVLL | 366208 | SCSSD |
| 225410 | QNEES | 298876 | ALLPS | 340401 | KRTQA | 366209 | RSWEH |
| 225426 | KARMD | 298877 | CREDD | 340402 | WETAL | 366221 | AASSP |
| 225428 | NFMKR | 298894 | EGNFT | 340409 | RPLFC | 366223 | SLCNF |
| 225430 | EETKK | 298901 | SPPSS | 340427 | DLFVF | 366225 | DGDLK |
| 225441 | SSEPN | 298902 | IARFY | 340429 | DTHCP | 366227 | YTEEA |
| 225474 | HLAQP | 298910 | RTSVE | 340434 | RARNL | 366228 | KLRLP |
| 225504 | TAIKT | 298912 | DVSRL | 340436 | TAAAA | 366230 | ELLTS |
| 225512 | VHTCK | 298919 | SVTTV | 340445 | AVKAF | 366231 | QPEDF |
| 225519 | NQKES | 298923 | LGTQC | 340450 | TTTNV | 366234 | QQATT |
| 225525 | QSMLS | 298925 | CLQNN | 340453 | SLKQM | 366237 | RQDPT |
| 225538 | NMRTS | 298942 | WLTFK | 340454 | LTVES | 366240 | HKEGK |
| 225550 | ASMGR | 298943 | IIYAD | 340463 | PVRGH | 366243 | ASKAH |
| 225567 | SAGSS | 298953 | LGSPG | 340465 | ILPYV | 366246 | QRLCR |
| 225573 | ERLAP | 298966 | PTITE | 340466 | SIHLR | 366249 | YCIGG |
| 225576 | ASGMH | 298974 | EQEGV | 340467 | EPGTD | 366258 | AQEKS |
| 225577 | LRMNL | 298992 | ITSRV | 340474 | EGPRS | 366264 | EATED |
| 225583 | VGTAP | 299001 | HLFYL | 340476 | FHLLG | 366267 | LPGIW |
| 225587 | TGKKP | 299022 | KRKIR | 340477 | LLDIC | 366271 | LQKLS |
| 225603 | KDDKN | 299045 | SPPTN | 340488 | INIII | 366273 | KKASH |
| 225609 | SLSFL | 299084 | HKAAG | 340494 | QRFLD | 366274 | GSCML |
| 225648 | EKQAE | 299088 | VEKDE | 340505 | TLAKK | 366275 | HENAI |
| 225655 | RRSQY | 299092 | PKVDS | 340507 | RQLLK | 366278 | PETPL |
| 225665 | LFLSG | 299106 | SSFVI | 340510 | VSGQK | 366280 | RKRFY |
| 225688 | RCVIS | 299125 | KKMRA | 340514 | MGEKV | 366283 | TFSQF |
| 225696 | NHVNF | 299134 | SGSQE | 340524 | KTIQV | 366284 | SFSCQ |
| 225698 | FVKSQ | 299136 | DKPTL | 340526 | WKYCV | 366288 | HSLSD |
| 225719 | YSSKH | 299138 | EGLIL | 340546 | ASSCC | 366291 | LGLQA |
| 225724 | LYAFH | 299140 | SSSDC | 340560 | TQLIF | 366293 | PRVLQ |
| 225726 | KVKAM | 299155 | AEAEA | 340563 | FSAHC | 366299 | RLLGG |
| 225728 | NTSGK | 299157 | IGGDF | 340568 | QFRRN | 366300 | TDEDD |
| 225729 | QIVKK | 299162 | PIALL | 340572 | MVSIS | 366306 | FEKPT |
| 225737 | KSTKL | 299163 | KGRYN | 340578 | TETTV | 366307 | TPQRL |
| 225760 | QAFRP | 299164 | VLRPC | 340584 | LKNKK | 366317 | FDRRD |
| 225768 | TPDAL | 299166 | VHYEI | 340587 | LKQSP | 366322 | KMTFL |
| 225777 | PPPVY | 299174 | SRPQK | 340590 | NTAHQ | 366325 | GFFVG |
| 225792 | TGYSQ | 299178 | IPVST | 340594 | LNMSF | 366328 | FLPCS |
| 225805 | LDQSF | 299191 | PSMAL | 340595 | PYVWD | 366330 | LANAL |
| 225823 | EEIAC | 299192 | KRLTS | 340596 | GFELS | 366331 | VDVET |
| 225831 | QTPKT | 299194 | ISSVA | 340608 | DILDC | 366336 | SWRAA |
| 225842 | PSKRK | 299198 | MPAQK | 340609 | DGREL | 366339 | HHLNQ |
| 225844 | HTLKT | 299201 | LWPPG | 340625 | CKLDF | 366342 | PFSLL |
| 225873 | YSPEN | 299206 | AERDW | 340627 | DLNEM | 366343 | VPMEI |
| 225893 | PLQAW | 299213 | SRACV | 340636 | GLKRR | 366345 | VIRRL |
| 225899 | PQGRY | 299218 | SMDTS | 340637 | LVTIH | 366346 | FPPPP |
| 225916 | GLIDK | 299229 | RGRLY | 340644 | KLKRK | 366347 | QQWTW |
| 225927 | VAGSW | 299233 | NGTRL | 340652 | GSALF | 366352 | TETLD |
| 225928 | FRLLH | 299234 | PLHAA | 340657 | KKHLG | 366355 | SKLTY |
| 225929 | PAAPQ | 299237 | AACLP | 340658 | MSLDS | 366363 | QVNTW |
| 225941 | VEPSC | 299238 | PPRDL | 340660 | TWVYC | 366365 | AMFKL |
| 225964 | PVCFL | 299259 | VSKLA | 340661 | EECIR | 366369 | LNPSH |
| 225972 | TDSQQ | 299272 | NHHDC | 340662 | SGWVM | 366372 | WEWGK |
| 225973 | SCNLM | 299290 | PRAES | 340667 | SDSAL | 366373 | MEVDV |
| 225992 | SPLDL | 299293 | TDLPM | 340668 | ALAEA | 366374 | LMGWE |
| 225995 | VSWVY | 299295 | SGMEV | 340671 | NLHQA | 366375 | FISMK |
| 226004 | GKLKP | 299297 | SPKPH | 340672 | PKSQE | 366378 | FFCKK |
| 226021 | AEPEH | 299299 | AVSMT | 340677 | PGRKP | 366380 | RNCKS |
| 226067 | RHGPL | 299300 | DHHPC | 340683 | WKDTL | 366381 | MEVDV |
| 226091 | IYYKV | 299308 | LQDQM | 340684 | LLPRS | 366383 | PNVLV |
| 226094 | ANLTS | 299314 | NRIRV | 340685 | DKLGQ | 366384 | ILTSA |
| 226102 | FSLFV | 299320 | PLEQL | 340688 | LVTSL | 366385 | CKEQV |
| 226105 | IFGPQ | 299322 | AFQTV | 340691 | FEMDI | 366387 | NEIEY |
| 226193 | KMKNA | 299326 | FVPVA | 340692 | YGYTI | 366390 | ARPGH |
| 226207 | IISEE | 299328 | LQPGR | 340693 | DALES | 366391 | FLAQK |
| 226209 | HESEE | 299333 | VPVEE | 340698 | KVGRY | 366392 | IEFVN |
| 226218 | APGHL | 299335 | VPGYN | 340703 | PPKSK | 366395 | SPVAH |
| 226225 | STHRD | 299338 | EEEEY | 340736 | DTYLQ | 366396 | RYNWN |
| 226230 | KRKKK | 299339 | KNAYV | 340737 | KKLKK | 366402 | PIVPI |
| 226247 | PVALA | 299340 | GNARK | 340742 | RKMDL | 366404 | METSP |
| 226253 | ANHAY | 299345 | SDKET | 340748 | MLKRT | 366405 | VTLSQ |
| 226258 | WPSIN | 299350 | YRRCA | 340752 | LNLSI | 366410 | AEAKT |
| 226272 | SKTGI | 299351 | FIYMA | 340770 | TRRIY | 366412 | SGVQT |
| 226279 | CTSEI | 299353 | NHLLK | 340776 | PTDLP | 366413 | FSATY |
| 226284 | YYHHQ | 299367 | NFLPL | 340784 | KTASA | 366415 | ILIYY |
| 226299 | SQDNA | 299370 | DLGFL | 340787 | ILGPL | 366416 | KYSSS |
| 226317 | GNKKN | 299373 | SAFHS | 340795 | TFGIP | 366418 | EKTKK |
| 226319 | SILAS | 299377 | RSLFA | 340796 | QPEEN | 366419 | RLVRD |
| 226355 | PKIGN | 299381 | TPSSG | 340797 | YRYLQ | 366425 | RRRKF |
| 226359 | AALGV | 299413 | EEEDT | 340799 | VAPSN | 366438 | QEWLV |
| 226382 | KSSMF | 299415 | KSQAD | 340807 | KRKKK | 366445 | HQPPK |
| 226413 | GYFSL | 299421 | KMQDK | 340811 | KKTIL | 366447 | THYPP |
| 226432 | PKYFL | 299424 | PHYFT | 340820 | AKQGL | 366453 | RDTEL |
| 226440 | YQSRE | 299427 | TLLNP | 340823 | EEKSD | 366460 | VSEQC |
| 226444 | FRTEI | 299432 | VDGGI | 340826 | KKKGK | 366461 | TLSGM |
| 226460 | PTPAP | 299440 | DSMEF | 340836 | DEDSD | 366463 | VSCLL |
| 226522 | VVEVS | 299441 | TELHI | 340838 | PPLQP | 366467 | QSMNT |
| 226524 | EHLES | 299443 | TKTNI | 340841 | SSHSS | 366475 | DSAQD |
| 226574 | LEGKI | 299459 | TRNFP | 340842 | AAQGS | 366476 | KRTGW |
| 226578 | LTDIY | 299466 | EIGIN | 340858 | WDRDM | 366477 | PFQVW |
| 226725 | IDKPL | 299481 | WWWCF | 340871 | LKKEK | 366479 | NWDMS |
| 226730 | ISTLT | 299492 | VGQIS | 340874 | EGWAT | 366480 | HQENS |
| 226760 | FLSAA | 299498 | MIFTY | 340879 | PESES | 366482 | SGKDA |
| 226798 | DRYCK | 299502 | RFSSP | 340883 | TAPAW | 366485 | QPMVM |
| 226840 | IKESL | 299505 | YLPQY | 340887 | SNYTR | 366487 | RIGVR |
| 226951 | HLLPL | 299512 | VRIYS | 340896 | PPWEP | 366488 | EFSEF |
| 227135 | KEENK | 299518 | VKDLD | 340913 | EETNR | 366490 | IKQIG |
| 227155 | KVPKY | 299529 | IYVRE | 340914 | KENSE | 366491 | VCVCI |
| 227157 | KELQF | 299540 | AGTTG | 340925 | EEEEG | 366492 | ELSSK |
| 227251 | AAPKK | 299543 | LNDLM | 340935 | CQDGY | 366499 | HCAKQ |
| 227256 | EITVS | 299550 | SISQV | 340937 | IAVKP | 366506 | TCQEL |
| 227266 | PIPKL | 299563 | MAGAK | 340943 | QTAAV | 366509 | KTLSQ |
| 227322 | LAPQR | 299565 | GNANK | 340964 | TKSDG | 366511 | PHHGV |
| 227348 | PESIV | 299575 | TVSNF | 340967 | ESDEL | 366513 | STLSY |
| 227349 | VLHIV | 299578 | THRIL | 340969 | EKGKE | 366515 | LQYFV |
| 227378 | IEEVD | 299596 | KQKFE | 340972 | YYSHA | 366519 | KCKST |
| 227451 | AQEKD | 299598 | ALPKD | 340975 | SFQDR | 366520 | TLIRH |
| 227471 | GPEEQ | 299601 | EEDDD | 340982 | EDRGW | 366522 | MEGVL |
| 227474 | IKSII | 299604 | LLQEE | 340983 | VIPGH | 366527 | ANKLY |
| 227507 | RDVDI | 299608 | EKKKD | 340989 | QEPQS | 366528 | RGQEC |
| 227520 | PAAKI | 299612 | DLQDP | 340995 | WSKSD | 366534 | SWCSL |
| 227524 | KFYSL | 299613 | SLTIY | 340998 | KRECK | 366537 | PNIKK |
| 227525 | SVEEE | 299626 | KTKKQ | 341006 | HKSSH | 366539 | PRLQE |
| 227618 | DQWMI | 299633 | TSEGT | 341013 | ANGIQ | 366544 | DESES |
| 227638 | LDSSC | 299641 | HSSLG | 341021 | RRVKR | 366549 | IDREF |
| 227665 | HLGDP | 299642 | FTIPQ | 341030 | NPISV | 366550 | KPRLY |
| 227666 | APLES | 299663 | KGKSL | 341031 | GSIGF | 366551 | LEGLL |
| 227752 | LQSSE | 299665 | GTTLN | 341032 | VFTCK | 366553 | RRPPH |
| 227756 | RSLAS | 299667 | IREST | 341043 | PSPPA | 366565 | HVGVR |
| 227758 | RTFLS | 299671 | ATQKS | 341044 | SPAST | 366578 | TGNAL |
| 227868 | PIRLA | 299687 | ELQEA | 341045 | KKKRD | 366579 | LDCFV |
| 227880 | SRDRG | 299694 | PERIV | 341051 | WGPGS | 366581 | YGFGG |
| 227918 | LCDLF | 299697 | RKHCP | 341058 | KELSL | 366582 | CFWWI |
| 228027 | VLEVN | 299705 | RAVHS | 341061 | GGTVA | 366586 | QNIKI |
| 228136 | KSSGS | 299709 | VWEMF | 341071 | GESNV | 366592 | FLVVL |
| 228140 | SALVA | 299714 | LKRES | 341082 | VVQYH | 366595 | QEHMQ |
| 228217 | SVEGF | 299721 | KCSVM | 341083 | PPGLL | 366603 | LDQKS |
| 228226 | TIAEN | 299727 | NCTHV | 341085 | DDWSN | 366604 | FRAHW |
| 228245 | KLACE | 299734 | LLLET | 341094 | KAKGK | 366608 | LPKCL |
| 228251 | QSSAE | 299736 | NMSKE | 341108 | NKLDY | 366618 | YTSSK |
| 228264 | KSASS | 299752 | LKATV | 341112 | NTKVF | 366620 | DAFLG |
| 228280 | EFQEV | 299759 | DLSVL | 341117 | HLGQV | 366623 | RATPT |
| 228284 | LFLRK | 299761 | LSARS | 341134 | PPFPF | 366624 | PQPSK |
| 228289 | ECGKA | 299764 | YPYTS | 341139 | KYQIE | 366628 | ELPST |
| 228318 | LGLTQ | 299766 | LSSRY | 341141 | APPLG | 366629 | VRLFT |
| 228327 | LESTL | 299767 | EKDEL | 341145 | TRFHC | 366630 | PRRRC |
| 228347 | SKYNE | 299798 | RGSRL | 341151 | RNIHT | 366632 | KLESK |
| 228434 | NKPYK | 299824 | CCRIS | 341159 | EMIPK | 366634 | FSWLL |
| 228437 | LSAHK | 299828 | DEFNR | 341165 | TGERP | 366637 | PRRRC |
| 228438 | RKRIH | 299847 | SKDFA | 341171 | APRIL | 366638 | KLESK |
| 228463 | HSFLP | 299853 | GTVQS | 341178 | SEPSF | 366640 | LESKP |
| 228468 | EDFTC | 299855 | SWLNC | 341179 | MCLHR | 366641 | VTSEQ |
| 228476 | PPSHL | 299866 | EIHQH | 341187 | AASED | 366642 | SHGSP |
| 228495 | AHLHQ | 299871 | IGEKP | 341199 | LMQSS | 366645 | DWAKF |
| 228506 | CLCRL | 299882 | QDGVL | 341206 | PEQIH | 366653 | QGGPC |
| 228510 | ALDGL | 299886 | SSMTK | 341208 | RGSLS | 366654 | GSLEA |
| 228515 | LPLAV | 299906 | VDVLM | 341213 | ARVVT | 366655 | FMFTH |
| 228534 | ATLSP | 299927 | LSPQV | 341214 | ATSKK | 366656 | KIMTC |
| 228567 | PPSTP | 299933 | PPTGN | 341217 | SNASR | 366667 | TRKKP |
| 228606 | QFLDR | 299952 | WSGTL | 341218 | SLSVR | 366671 | HAYKK |
| 228641 | EVVEK | 299957 | FNDVL | 341220 | CHLLH | 366672 | SEREP |
| 228644 | IYHVL | 299964 | LSRPL | 341223 | DSDSD | 366673 | TLSGL |
| 228682 | LNSSA | 299974 | LHVSP | 341227 | DFGNY | 366676 | IKGEV |
| 228705 | GNKLS | 299977 | LKASV | 341236 | IHTGE | 366677 | DQVCT |
| 228740 | DLKVD | 299989 | NLEDK | 341237 | IDTGE | 366679 | HHKAK |
| 228741 | NSQKS | 299992 | LGHLL | 341243 | LFIQT | 366682 | ITNLS |
| 228799 | LQDPT | 299997 | CHVDF | 341247 | VPEKD | 366688 | QLRVS |
| 228811 | EQPLW | 300006 | QCNIM | 341253 | PRPHF | 366689 | VDILV |
| 228820 | LIDFH | 300013 | LKVQT | 341256 | KTYTV | 366690 | PMFIW |
| 228825 | SGPGQ | 300022 | DNGWD | 341261 | GAPQP | 366693 | PRRPC |
| 228827 | ECQDE | 300026 | AIAKE | 341266 | PFPRS | 366696 | GDTIC |
| 228837 | FLPRI | 300030 | VLEPD | 341267 | DNEGE | 366697 | SRKTI |
| 228841 | GEEKD | 300035 | NDEKE | 341268 | NGGLA | 366699 | LLLNV |
| 228843 | NATST | 300051 | PGKVL | 341271 | GGGWL | 366702 | YVVSP |
| 228850 | LMVNL | 300055 | LRKKS | 341274 | SGKKP | 366704 | LFSEK |
| 228858 | LLTDV | 300056 | KLKTR | 341278 | QKLFP | 366715 | EMSPF |
| 228862 | IIEVS | 300057 | PEEPK | 341280 | ASSSS | 366720 | THSLS |
| 228865 | NSNSW | 300060 | TENSK | 341282 | IKELF | 366727 | KKEEC |
| 228887 | DAGGV | 300061 | MLDEL | 341285 | SGRRF | 366729 | GGCEL |
| 228918 | FITHD | 300069 | YRQFC | 341289 | EEEVA | 366731 | LWEGI |
| 228922 | VCALT | 300079 | NLVPF | 341291 | QSHKD | 366734 | LFFFG |
| 228928 | VKAAS | 300086 | EFRKK | 341295 | TRRRR | 366736 | KKEEC |
| 228929 | FQKVE | 300087 | PLTQV | 341302 | TWDGG | 366739 | PMAIT |
| 228936 | SKSRV | 300093 | RLKAS | 341308 | PGVGS | 366740 | PERSD |
| 228938 | RRGTK | 300098 | PLTPS | 341309 | VREEG | 366743 | GWRHM |
| 228945 | KEWTE | 300101 | KNPDS | 341314 | GEFLY | 366745 | VVGPL |
| 228955 | LKVSA | 300105 | RTTPV | 341324 | ITFDD | 366746 | QKALL |
| 228958 | IKLYL | 300107 | DAANS | 341327 | PEQQC | 366747 | HPGNP |
| 229002 | RRKSV | 300113 | IRILK | 341342 | GAVSV | 366751 | SSISS |
| 229003 | IVSST | 300119 | EVTVQ | 341346 | PDAVC | 366752 | HPGNP |
| 229022 | GNEIS | 300127 | SRKWG | 341351 | KKLKT | 366754 | LFSEK |
| 229030 | SPTCV | 300128 | NNFLT | 341360 | AQDGH | 366755 | VEDSC |
| 229043 | IVAEA | 300131 | EASRQ | 341361 | IANTV | 366758 | QEHMQ |
| 229088 | GTKKR | 300134 | ANPSW | 341363 | CQTLI | 366760 | DFLYP |
| 229134 | LESSQ | 300145 | YYSNI | 341364 | PDFDD | 366765 | LFSEK |
| 229135 | RRASQ | 300146 | VQGIR | 341368 | RKATS | 366766 | EKEYE |
| 229179 | YEIQL | 300147 | AQQKL | 341380 | SNLTL | 366770 | LHPVI |
| 229195 | AQQAF | 300151 | MPKRV | 341382 | SKGLC | 366772 | PMAIT |
| 229201 | DDEDD | 300161 | GEGEN | 341409 | RTCRK | 366773 | PMAIT |
| 229214 | KKKKK | 300167 | RAKQK | 341412 | LSTLK | 366776 | PHLGF |
| 229238 | HGKFR | 300175 | DKDPE | 341416 | FPLCP | 366783 | SEQPF |
| 229239 | MASKE | 300176 | TNPFL | 341422 | ARVEL | 366785 | RMGIS |
| 229243 | LGQFG | 300177 | SIDLD | 341427 | KWNPL | 366786 | DPLSG |
| 229251 | WSKSN | 300179 | WDTAI | 341442 | VKIMK | 366787 | KKEEC |
| 229264 | LKIWN | 300181 | NGPSV | 341443 | HTILP | 366788 | KGQGT |
| 229265 | PLVES | 300182 | WKQTV | 341451 | TASLY | 366789 | IQVDD |
| 229266 | QNNMD | 300184 | SRSWI | 341455 | SDTSN | 366794 | AQTSC |
| 229268 | QRVAS | 300187 | NPLTG | 341466 | RSPPW | 366797 | KSTQF |
| 229270 | INAKQ | 300190 | CEQCC | 341467 | GEDDS | 366800 | IDTDG |
| 229274 | YLLYQ | 300192 | NIGIN | 341473 | KELEK | 366802 | PILSS |
| 229277 | NPSVL | 300209 | EPRPA | 341479 | LTLPP | 366811 | SCPWL |
| 229281 | SVAAS | 300213 | SELEL | 341481 | DKLGQ | 366813 | ALSAA |
| 229304 | SVAWR | 300215 | PELSA | 341483 | TTGST | 366814 | PRRRC |
| 229307 | QPEDV | 300226 | IQANK | 341489 | INVCL | 366821 | NWQSN |
| 229314 | SNNMV | 300233 | EKGKS | 341490 | EAKEY | 366822 | PAWAT |
| 229319 | DLKDL | 300245 | KTVAT | 341497 | QKLHE | 366826 | SRKKI |
| 229328 | LYKPI | 300249 | QQEEY | 341502 | PQMKG | 366827 | KCEVA |
| 229329 | NSCQK | 300255 | MGQFY | 341504 | LLPYA | 366828 | EWLEF |
| 229330 | KAPLN | 300258 | RRGGV | 341522 | KKEKN | 366829 | FNEAQ |
| 229332 | MKIHL | 300283 | IHTKH | 341528 | EDFSM | 366833 | FMFTH |
| 229335 | RTLGL | 300289 | AQEDL | 341539 | GEAIK | 366836 | SSISS |
| 229340 | KKRCC | 300291 | NFIYN | 341543 | AKDSL | 366838 | VIETV |
| 229379 | GYEDE | 300302 | PAIAN | 341549 | TKPAG | 366839 | PERSD |
| 229384 | PFPHS | 300303 | LEADI | 341550 | RGCGG | 366840 | PMAIT |
| 229390 | PFRPY | 300398 | SHIRN | 341551 | PQPLD | 366843 | HQQQL |
| 229395 | SLRKS | 300399 | GKSAP | 341552 | GDESY | 366844 | GFGKS |
| 229402 | VYPDS | 300404 | LQCAA | 341562 | CMTSQ | 366849 | LWEGI |
| 229416 | TDSSN | 300406 | GCLIS | 341564 | FCSKK | 366850 | GEFLY |
| 229447 | GPPRT | 300408 | LQLPQ | 341565 | EPMEC | 366853 | GFGKS |
| 229554 | EDDYD | 300413 | GGPRR | 341572 | EKYIL | 366856 | GKILK |
| 229563 | FNRPH | 300415 | EDNYL | 341581 | AKGSK | 366863 | IGNKP |
| 229570 | TSKWD | 300417 | IYHSS | 341584 | RFSSP | 366867 | GEFLY |
| 229595 | HMDCM | 300433 | DNPAF | 341597 | PLLEH | 366870 | LWEGI |
| 229606 | WSVFS | 300434 | VIESH | 341610 | QRVTK | 366875 | LWEGI |
| 229633 | EKLRT | 300441 | RHLNL | 341625 | YMAED | 366876 | KLFLD |
| 229702 | LWLLH | 300452 | VQGLA | 341633 | AQTRL | 366880 | LGMAA |
| 229707 | TRPHS | 300456 | GEEKL | 341634 | ASREP | 366883 | RDTQM |
| 229708 | ILAGR | 300458 | AHLTD | 341636 | MDTGR | 366886 | PDELH |
| 229715 | LQQKQ | 300504 | KDFFL | 341637 | LLPLQ | 366888 | KLYKV |
| 229725 | VYGTL | 300515 | NITII | 341640 | GSRSS | 366898 | SAQAD |
| 229758 | NLRRL | 300525 | TWIYP | 341641 | VRDTM | 366901 | HLMLT |
| 229769 | KHLGP | 300527 | IRWIC | 341652 | KQAAG | 366903 | FRAIN |
| 229771 | KLACE | 300557 | GRSSK | 341657 | EKEPS | 366908 | GGGWT |
| 229794 | EEMES | 300571 | GRHLW | 341662 | SHPWK | 366915 | KRHQN |
| 229812 | MKAAK | 300574 | DEDFS | 341674 | VGSDP | 366918 | RVVLE |
| 229824 | QQLAQ | 300575 | FQKGA | 341677 | KRAWP | 366921 | WEVQL |
| 229829 | SSVPR | 300576 | VMQDT | 341681 | PEFSV | 366922 | YVACT |
| 229854 | IIFLI | 300584 | KSGWS | 341682 | RLSRT | 366923 | PDDRM |
| 229903 | PLAGR | 300589 | TRLLL | 341684 | QKEQK | 366924 | LKAFH |
| 229922 | AEIMA | 300605 | LQHLL | 341692 | HCAYL | 366926 | RSDLI |
| 229955 | HRTVV | 300618 | HQTYF | 341700 | GQNFG | 366927 | PQKNS |
| 229971 | KSFTQ | 300619 | KPLLY | 341715 | QVVWR | 366934 | CRKMK |
| 230012 | DRDSL | 300647 | RGVPY | 341719 | SPSLT | 366936 | AVRTL |
| 230036 | SLGSD | 300648 | DTILT | 341723 | SQSVR | 366939 | PEYSE |
| 230048 | KHLGN | 300650 | CPTLK | 341734 | AESKL | 366942 | TGVHA |
| 230050 | FKCKK | 300651 | ALIGN | 341737 | NWVQF | 366948 | QPSCC |
| 230053 | VKIEV | 300658 | KFKLD | 341742 | FPPGF | 366950 | QPSCC |
| 230085 | KIRHA | 300659 | RSDGL | 341745 | APCSV | 366951 | HPAPM |
| 230099 | PPPGD | 300671 | TMNPG | 341748 | SPGAP | 366953 | YQPKF |
| 230122 | GQEQS | 300681 | TELLL | 341756 | GPAED | 366955 | SGSCC |
| 230124 | RNRYL | 300682 | YSAPA | 341762 | WKYCE | 366962 | YTSSK |
| 230215 | FFCKK | 300688 | IGYDV | 341765 | TVQQR | 366970 | NGPEA |
| 230221 | AMFKL | 300692 | WARNK | 341779 | TVLYE | 366974 | YGFGG |
| 230223 | SKLTY | 300714 | AFLNA | 341782 | KKKYI | 366975 | YLVPL |
| 230236 | GGLFM | 300737 | KPLKK | 341785 | DDTLL | 366976 | CQPSC |
| 230256 | IQTKM | 300738 | LMCGS | 341787 | PVSWG | 366978 | CEPTC |
| 230260 | GPMQH | 300747 | LDGED | 341797 | ESDDG | 366979 | AARAP |
| 230301 | RGNRR | 300762 | SSDIP | 341803 | GLYCE | 366984 | ENCCL |
| 230321 | LCFSS | 300773 | HRIGT | 341815 | ACISG | 366988 | LFKNN |
| 230340 | PITVE | 300778 | NMHSR | 341819 | ECRQM | 366995 | ASTAL |
| 230354 | FRKTT | 300784 | RRLLK | 341821 | ELQAM | 366997 | MKLGK |
| 230361 | KSAMF | 300793 | SRRGT | 341823 | LSFSA | 366999 | RGERA |
| 230381 | APEAG | 300809 | PEPAQ | 341826 | SGRRF | 367004 | KLDSI |
| 230402 | SQEAL | 300835 | QPHWT | 341828 | CCNWA | 367005 | LEDGF |
| 230419 | VDSKP | 300849 | EEGPA | 341831 | SFLRQ | 367013 | LQRGP |
| 230431 | PDPTT | 300850 | LSFSL | 341838 | KKFES | 367019 | IREEG |
| 230449 | TMMKT | 300870 | TREKL | 341841 | PRRRR | 367021 | DILYK |
| 230495 | RGERA | 300873 | FCVLL | 341843 | PEISP | 367023 | RRKIT |
| 230510 | KRGKK | 300900 | AGFLR | 341848 | FTGHL | 367024 | GGCCH |
| 230538 | SCPAA | 300901 | GVLEL | 341867 | FPTKP | 367028 | AEQTV |
| 230565 | PLLQA | 300917 | KAVTQ | 341874 | LLYDE | 367034 | YGFGG |
| 230568 | TIMCS | 300935 | RCVLL | 341882 | IQPLI | 367037 | QPPPP |
| 230588 | QRPRK | 300952 | EFVVA | 341885 | AVATT | 367046 | QCSSP |
| 230640 | ASLYL | 300954 | LPAGT | 341890 | LGLVA | 367049 | ENMTS |
| 230643 | KVSAL | 300961 | GKGRD | 341905 | LDQTT | 367050 | KKKAE |
| 230658 | SPIEA | 300965 | VPGGT | 341909 | QAQRS | 367055 | KLTWS |
| 230671 | EESMM | 300976 | AGTRR | 341912 | RPMKH | 367059 | DIAKY |
| 230732 | YSAVH | 300983 | PGKPK | 341914 | EFTSS | 367064 | LTWDS |
| 230771 | RLSES | 300992 | RQTTR | 341924 | LQLKT | 367065 | LWRPW |
| 230792 | INPNL | 300996 | HKKIH | 341926 | QAKQQ | 367066 | APHTG |
| 230859 | VSLSR | 301007 | RGFLP | 341934 | RSEMK | 367070 | EKGHS |
| 230882 | NKIMP | 301011 | IPGKA | 341938 | EAKYY | 367075 | VIKQP |
| 230902 | FGKKY | 301012 | PKPAA | 341942 | FWARL | 367081 | TLTRL |
| 230914 | QLLEN | 301015 | TREKE | 341944 | SDILK | 367086 | AEPQP |
| 230990 | MTNSH | 301019 | AEEGL | 341947 | CAMNC | 367096 | KEHQL |
| 230993 | NISVN | 301021 | MIQVC | 341952 | DCVLG | 367098 | EGVLY |
| 231004 | TISPY | 301031 | SHLKE | 341961 | PRRPC | 367102 | EKNGL |
| 231009 | PPHTN | 301037 | MGERG | 341963 | IVVPT | 367104 | GEKSS |
| 231021 | DSDRD | 301038 | QNLVY | 341966 | HRKAL | 367111 | EDVKR |
| 231061 | KDLVI | 301039 | NPNLS | 341974 | LYEVM | 367124 | FPYAA |
| 231121 | LELNQ | 301042 | GTSVF | 341985 | ISFQS | 367127 | FSFFP |
| 231130 | SFEFS | 301043 | PTCCP | 341987 | HHIKI | 367130 | GDDLN |
| 231134 | SFGLN | 301046 | LCEKL | 341988 | FPPTK | 367137 | TFTHL |
| 231136 | SFPFS | 301050 | PKDSY | 341992 | PSMST | 367139 | HFGKC |
| 231137 | NNLGF | 301057 | GESSE | 341998 | RFSSS | 367142 | YVNPC |
| 231173 | SEFLE | 301061 | VNVCK | 342002 | TGEKP | 367145 | AAKNR |
| 231189 | AESGL | 301067 | RKWMN | 342007 | RFSIN | 367151 | VAVRK |
| 231198 | LDEDS | 301068 | RCHLM | 342008 | KVTTF | 367157 | APVLP |
| 231228 | SVPCS | 301071 | EGEEY | 342011 | LFDEI | 367158 | TDIAY |
| 231238 | PNNNR | 301072 | EGEEY | 342015 | SEAKK | 367159 | TDIAY |
| 231283 | QKIFL | 301073 | KGEPA | 342019 | TAWMQ | 367162 | TDIAY |
| 231357 | KPFCA | 301093 | TGKKS | 342021 | EKPCE | 367163 | TDIAY |
| 231420 | ERRAK | 301096 | SNTCN | 342023 | EDDYI | 367164 | TDIAY |
| 231449 | SKCSS | 301141 | SFLPR | 342026 | YTPQP | 367166 | HGLLV |
| 231454 | WIIES | 301146 | SFLPR | 342032 | KQAKK | 367172 | DTVEN |
| 231461 | KCVKQ | 301149 | HPEHM | 342033 | EEDVE | 367173 | REHPT |
| 231482 | QQTTL | 301159 | MYERQ | 342050 | QQGLP | 367177 | ANENV |
| 231484 | SSRCL | 301171 | LIRMQ | 342053 | DGSGD | 367185 | VGLSA |
| 231487 | WCEEK | 301173 | AITST | 342056 | DSKSG | 367187 | DCGQL |
| 231498 | LERLH | 301178 | QEDGA | 342059 | LENRP | 367189 | GGRSP |
| 231504 | PDYFL | 301180 | VAYNM | 342070 | YWEKI | 367193 | AELER |
| 231509 | LFHQK | 301190 | RMAQK | 342071 | IKGIM | 367196 | LPPKK |
| 231512 | NDYIF | 301200 | DVIGL | 342082 | SPVKA | 367197 | LKINP |
| 231524 | VLDVA | 301202 | GFDAP | 342083 | DSDLE | 367198 | ILGMV |
| 231526 | IKVES | 301204 | PLRDL | 342087 | RVYFQ | 367201 | KLNKI |
| 231572 | PVQRM | 301215 | DTAFV | 342097 | LLPPT | 367202 | LWTYN |
| 231589 | ISSVS | 301218 | SNFHV | 342098 | TTRPL | 367203 | PSASS |
| 231656 | EEFLP | 301219 | AAAGR | 342100 | ARHFS | 367207 | RNSCA |
| 231668 | LFPFL | 301226 | LEAQA | 342102 | HISIA | 367208 | EWKKY |
| 231706 | DYSPE | 301233 | KIHTH | 342104 | VATGI | 367212 | ASCTD |
| 231721 | EVEAT | 301242 | RTAHP | 342105 | PKLVS | 367214 | QICNQ |
| 231749 | GDRAK | 301244 | NTYVL | 342107 | HLASE | 367216 | VPSAG |
| 231751 | EFLRK | 301246 | KEAPH | 342109 | NPIGK | 367219 | SRASR |
| 231790 | VFERC | 301249 | SKTGV | 342114 | AIHGG | 367220 | GVTVR |
| 231887 | PSSKL | 301258 | GPGQL | 342118 | GRSLP | 367223 | FRTVL |
| 231908 | PMAGG | 301260 | LHRSS | 342121 | IANNG | 367224 | PQQHF |
| 231948 | VILCL | 301263 | LAPSL | 342137 | VQLRE | 367226 | LELEA |
| 232003 | HTFPK | 301264 | ECGLR | 342143 | EDTEM | 367228 | VENKA |
| 232014 | LPKAC | 301272 | KVGEE | 342148 | VLFIK | 367229 | PCEGV |
| 232125 | KAKTE | 301280 | PLGAS | 342154 | RRRLS | 367244 | LTNRN |
| 232165 | GFGML | 301281 | IEKLL | 342156 | PVPGG | 367246 | ATGVS |
| 232217 | VFKKK | 301284 | LDEES | 342162 | PGDPF | 367247 | WREAE |
| 232219 | FKKVQ | 301286 | PAGGQ | 342169 | EWGNS | 367248 | ATGIF |
| 232375 | VPAHQ | 301293 | APPVP | 342179 | QCREM | 367253 | SQSRR |
| 232424 | RPWRN | 301295 | TRVEI | 342181 | GKGGW | 367255 | ATSDL |
| 232461 | DCGLF | 301305 | DDITF | 342188 | VKGKP | 367258 | GKIFK |
| 232496 | ILYEV | 301318 | SLPSP | 342190 | EQPQV | 367263 | STKKE |
| 232501 | IICWK | 301323 | LQVAR | 342197 | PRGEM | 367265 | IHEKV |
| 232508 | KRIQP | 301327 | PSTFL | 342205 | SLLPF | 367276 | EADTT |
| 232519 | NEAQR | 301332 | CWQRR | 342207 | PIRQY | 367282 | DEAHS |
| 232564 | LRIWN | 301336 | AAPEA | 342216 | LQEEP | 367284 | LGLAS |
| 232603 | KTSEN | 301364 | QGGME | 342222 | LLQSN | 367288 | SLYYS |
| 232607 | SRLGV | 301391 | CSFPL | 342228 | ASPRS | 367291 | GEAQI |
| 232744 | IGLDC | 301396 | TEPDS | 342235 | RNTVV | 367298 | AKPCI |
| 232766 | PLLTI | 301399 | DIVSE | 342240 | GKPQL | 367299 | FMTQS |
| 232854 | RRSGP | 301406 | RLPGP | 342254 | EGAMY | 367300 | KPSSL |
| 232863 | VRNIK | 301408 | PILPQ | 342259 | AAIKL | 367301 | EECNS |
| 232888 | PAAGS | 301411 | RTGRA | 342267 | QLEAY | 367304 | EVKKQ |
| 232892 | LKENL | 301419 | ELLLV | 342268 | TRRLD | 367308 | GDVGD |
| 232905 | KVHGF | 301420 | IAENS | 342276 | PPFPT | 367309 | LLVRV |
| 232907 | LPSDS | 301439 | PEGPS | 342278 | SKRNF | 367310 | FLSFN |
| 232974 | MNANN | 301446 | SEVSE | 342291 | REDNV | 367314 | KFTNK |
| 232975 | MKGVE | 301452 | DDKDC | 342295 | TSDEN | 367315 | EEEDL |
| 232978 | SSRYS | 301455 | AEAAS | 342300 | FIMVN | 367316 | KTPEA |
| 233025 | HAKNN | 301457 | DQPYL | 342307 | IPELQ | 367318 | STITI |
| 233027 | ENMNF | 301458 | KTSLP | 342313 | SQPLC | 367319 | TPLST |
| 233047 | GLYQE | 301459 | PAPSL | 342322 | TPLLL | 367323 | KDYRS |
| 233055 | GFSPH | 301463 | HPLSG | 342333 | RGKTH | 367330 | QEELK |
| 233057 | ERHTC | 301464 | TGSSG | 342335 | PETSV | 367343 | NLSPE |
| 233072 | AGKAA | 301466 | WSCHT | 342336 | PGVPP | 367345 | EKNNS |
| 233078 | HPYRR | 301475 | HNRWI | 342343 | PTEHA | 367346 | GKKSQ |
| 233084 | LFRTF | 301480 | HLKTH | 342356 | SNSRG | 367348 | KHAGE |
| 233092 | QHDEL | 301488 | RNART | 342374 | LIAGK | 367349 | NEQLF |
| 233099 | KTSFF | 301522 | FSKHN | 342375 | VIFRS | 367352 | RTETQ |
| 233114 | FLSSA | 301529 | SFKSM | 342380 | NSPIS | 367355 | LLSVL |
| 233121 | DQDEY | 301532 | KVDSS | 342381 | ALFFL | 367356 | AFNIK |
| 233139 | IPFIF | 301547 | HKKTH | 342385 | AARHL | 367359 | PYMPE |
| 233143 | RSEIS | 301585 | RVDMD | 342391 | LKNPQ | 367360 | GRWRL |
| 233146 | IKVTT | 301587 | PIENL | 342392 | LGIFY | 367361 | KEEKL |
| 233154 | VRALQ | 301599 | GKVWV | 342394 | LNLSS | 367362 | FFLIK |
| 233156 | FVKNM | 301607 | PRSLR | 342409 | VHEED | 367363 | YLWKL |
| 233161 | YKGPM | 301624 | SGESL | 342411 | LRPLA | 367365 | NVSHK |
| 233190 | EPSIC | 301633 | LAAMD | 342413 | CPITL | 367366 | LKIMS |
| 233202 | GETSG | 301634 | CSPAN | 342422 | MTALL | 367369 | PLEIH |
| 233242 | LTIIL | 301645 | KFKHL | 342423 | AESGT | 367370 | QSCDP |
| 233270 | GLLLN | 301647 | HFSGR | 342426 | DQGVH | 367372 | SYLDS |
| 233336 | AFIKL | 301653 | QGQSS | 342433 | CHSIA | 367373 | FEFWL |
| 233379 | INKVV | 301656 | QRVSS | 342434 | RRQKK | 367378 | SSKST |
| 233468 | TDPPK | 301659 | LTKAS | 342445 | ASNYV | 367382 | PLRPA |
| 233535 | EPPQA | 301665 | TFTST | 342447 | LMVNT | 367385 | GLVLQ |
| 233545 | KAHRL | 301671 | ERVVS | 342448 | NKILF | 367391 | LAPVA |
| 233552 | KSSDQ | 301677 | QSLMD | 342466 | KKNNF | 367394 | SVFIP |
| 233557 | VTVSS | 301678 | YQSEA | 342470 | WDVHE | 367398 | KVIYQ |
| 233573 | KSNDD | 301686 | IFRKN | 342480 | ADVEP | 367406 | PSLSD |
| 233575 | GDEDL | 301691 | LENAY | 342481 | SKLTS | 367414 | ASWLL |
| 233604 | ITDRK | 301694 | LGIRW | 342490 | SKLGT | 367415 | VNARL |
| 233607 | ATLLE | 301695 | LITAC | 342499 | GPVLM | 367417 | PAACR |
| 233609 | FIPLK | 301698 | SHRSV | 342501 | SYSLA | 367422 | DSIFY |
| 233612 | GTMAI | 301701 | GFLLQ | 342502 | GGKKD | 367428 | AESTP |
| 233616 | MAEDY | 301711 | QPRKR | 342509 | EEPSS | 367429 | FEMMI |
| 233623 | LSQAR | 301714 | RPAEA | 342510 | VGVTV | 367432 | FEISC |
| 233638 | LASVV | 301717 | RCRRT | 342512 | FRRKG | 367439 | KATPK |
| 233668 | SEGST | 301719 | RNMCN | 342513 | EEDEE | 367440 | LDIIL |
| 233685 | LSQND | 301724 | LHLRS | 342518 | SIPLA | 367446 | DIERL |
| 233699 | EGTLD | 301727 | QTVIV | 342520 | LLLPS | 367449 | SYFLV |
| 233710 | IVFDK | 301729 | KEEKG | 342525 | SPSIS | 367454 | VRKRQ |
| 233712 | LNRYY | 301730 | NSTYA | 342528 | LSMVL | 367459 | CSKDR |
| 233714 | PAFEL | 301738 | RGSRM | 342529 | NTGSR | 367462 | SISNF |
| 233735 | CKFKN | 301740 | SSRSP | 342538 | EEASR | 367471 | LIFYA |
| 233741 | SGRKH | 301744 | IKNTT | 342546 | FAVRN | 367474 | TTTTM |
| 233809 | TQRMQ | 301749 | CTVEM | 342554 | LSVGK | 367476 | TYAKR |
| 233813 | SSNVE | 301761 | FEKPR | 342557 | TRTSH | 367482 | GFQVS |
| 233826 | TGLTE | 301764 | LTRIH | 342563 | LPSSS | 367486 | KIYAY |
| 233838 | VHSEF | 301765 | AWPGY | 342564 | PTMGR | 367491 | PLLNP |
| 233840 | EYLPQ | 301776 | ITDLP | 342570 | KGLML | 367495 | SMDRR |
| 233893 | GKYVD | 301781 | TCSSS | 342576 | ATKPS | 367497 | RILEI |
| 233944 | DALGA | 301785 | YQGYR | 342589 | RQSIF | 367498 | VTVVR |
| 233946 | HVPLG | 301788 | LGLVS | 342595 | EKLYK | 367499 | RSVMD |
| 233948 | LSLCL | 301790 | KPITA | 342602 | HLQNY | 367510 | KEYYV |
| 233954 | AAQKQ | 301807 | KQRKY | 342604 | RYFTF | 367527 | SLLDT |
| 233957 | VLSES | 301819 | RSLRP | 342606 | FNEVG | 367528 | AANPE |
| 233969 | GSEKP | 301825 | AVDSD | 342609 | SVKIT | 367530 | IMQNF |
| 233997 | GPGPA | 301831 | QAVGH | 342610 | AQAGS | 367543 | GKCIA |
| 234038 | TFVRF | 301838 | TSEAS | 342623 | ALGSF | 367545 | EAEIH |
| 234071 | KSWAP | 301873 | PQRRR | 342626 | ELEYL | 367550 | GPIKP |
| 234091 | KALCG | 301885 | PGSPA | 342628 | RAHLS | 367555 | RAAGE |
| 234111 | ASINV | 301886 | SQLPP | 342667 | IQTEI | 367563 | KKEIS |
| 234142 | TGRHI | 301887 | AQVHF | 342680 | TPPEP | 367564 | QDCSH |
| 234160 | ASESP | 301891 | ESTSL | 342681 | GAQRL | 367570 | SSLSY |
| 234170 | KKQRK | 301896 | ASLGF | 342692 | KITVI | 367577 | FTGLG |
| 234179 | MEEDP | 301904 | SQSFY | 342697 | AHSTL | 367585 | VLLPA |
| 234195 | TKEEL | 301905 | LETDV | 342699 | EVINK | 367590 | GGDSA |
| 234198 | AGTIF | 301908 | QNGNV | 342704 | ARAAS | 367591 | SVTGL |
| 234256 | KESVL | 301917 | ASASW | 342709 | PMRLY | 367593 | LELKA |
| 234296 | EEEEA | 301919 | VQTHF | 342710 | RRILE | 367595 | GKIGF |
| 234301 | QPKNK | 301920 | KLSGN | 342711 | LWAGP | 367597 | LELSA |
| 234313 | SRTGK | 301921 | SYSVS | 342719 | LQHSK | 367598 | PLFVE |
| 234347 | AKGRP | 301924 | EELDG | 342748 | VMSTI | 367601 | RDKAR |
| 234371 | DNAPL | 301939 | LALAL | 342755 | GVEEI | 367605 | LELSA |
| 234388 | VPSEP | 301945 | PRPSV | 342779 | KVALR | 367608 | AETGA |
| 234389 | PGSQE | 301956 | ETGQM | 342780 | RPQAP | 367609 | WFSPY |
| 234392 | KKANT | 301962 | YGQGD | 342787 | PTKSS | 367615 | LLQYE |
| 234396 | PDTAL | 301972 | SAVMY | 342788 | LKFTS | 367618 | LRGRK |
| 234420 | LIKEL | 301974 | NSLKN | 342790 | DKLGQ | 367619 | MLWAL |
| 234453 | PSFSS | 301995 | VRITG | 342793 | MEVWT | 367623 | IEIAS |
| 234454 | DFYDK | 302005 | NSGCC | 342797 | KRLQI | 367625 | IPMLN |
| 234590 | NPLAK | 302021 | DMVEQ | 342804 | AKYTT | 367629 | VETDV |
| 234626 | KDMSL | 302028 | QTGLS | 342805 | PEIAS | 367631 | AALKT |
| 234701 | QLSIA | 302037 | LWFRR | 342810 | LSPAG | 367633 | SVKPS |
| 234739 | GNMMF | 302046 | LDSIL | 342812 | QMKDQ | 367635 | EAGQS |
| 234798 | FPAPD | 302051 | KCSVW | 342817 | PDECV | 367637 | ILTNR |
| 234816 | IKPID | 302057 | RKTME | 342818 | GVWIA | 367649 | FVIEQ |
| 234923 | LDMDL | 302077 | CRRLT | 342828 | GIRAW | 367650 | SKIHS |
| 234961 | GGAAA | 302079 | NSTTV | 342830 | EFEAM | 367652 | RGSVP |
| 235090 | ASVTE | 302100 | SYVQI | 342831 | RFAPY | 367655 | TLRSY |
| 235180 | AQTRL | 302105 | VTDLY | 342832 | DNGWE | 367658 | QRRCF |
| 235290 | EKKMT | 302107 | MDTGR | 342836 | LFSMK | 367660 | GVGCP |
| 235307 | EEDIT | 302108 | MTDDK | 342840 | TQAGS | 367662 | QSLFS |
| 235329 | LQPSR | 302111 | PAGRN | 342847 | GRGQY | 367664 | VSVLM |
| 235332 | PRQKP | 302114 | QQQFV | 342848 | QGDQP | 367668 | RCRVW |
| 235345 | GKGAV | 302120 | HYVTS | 342850 | VMNPV | 367672 | LMESV |
| 235347 | FHLCS | 302140 | TEGSD | 342858 | TSGWV | 367673 | ESGRS |
| 235349 | DQYCC | 302144 | SDSES | 342859 | SWGDG | 367676 | QKLTQ |
| 235372 | PFFKE | 302160 | PLRVG | 342868 | TAIRS | 367678 | LCCLP |
| 235382 | EPHAT | 302166 | RRKSV | 342875 | LGVNY | 367680 | SGKKA |
| 235439 | WKIKS | 302177 | SSLKQ | 342876 | EHTFH | 367681 | RPASV |
| 235521 | LVGFL | 302189 | HVLDV | 342886 | YTSSK | 367685 | ITDQA |
| 235547 | KSKTQ | 302194 | YRISA | 342889 | VFDAR | 367689 | LMVLK |
| 235628 | APSAQ | 302199 | ALRKF | 342904 | PLVGT | 367697 | KIRMD |
| 235657 | FSNVV | 302202 | MTDVE | 342905 | SSQQA | 367700 | ISSVK |
| 235790 | APSRK | 302204 | ELQLL | 342906 | CFAME | 367701 | TTDDK |
| 235799 | EHMRM | 302216 | SDEAS | 342907 | QKKDY | 367702 | PKTEL |
| 235835 | PNYFR | 302227 | WKVEL | 342924 | YLLSQ | 367704 | MEGDD |
| 235932 | SWSCC | 302232 | RGAPW | 342931 | HLECR | 367705 | SDNYW |
| 235933 | ALQAL | 302234 | PTPKP | 342935 | SIPDA | 367707 | QISKT |
| 236040 | LGSSS | 302239 | TDVAT | 342950 | HKELH | 367712 | YLFRG |
| 236051 | KNRTH | 302251 | EQARP | 342952 | SNVAS | 367714 | LWRPW |
| 236067 | VKMGD | 302260 | AFEAD | 342962 | NRDFD | 367715 | YAAVA |
| 236130 | YCNLQ | 302269 | YSEYC | 342970 | RREGS | 367721 | YRRTL |
| 236137 | QVTTS | 302274 | ASELL | 342972 | LEKQN | 367727 | EVPAE |
| 236147 | MNDPY | 302276 | EEPGT | 342974 | RMHNR | 367730 | STLGG |
| 236166 | TLTFY | 302289 | ESLGQ | 342979 | EQRAE | 367737 | LECSQ |
| 236192 | MKYRT | 302297 | VDLSA | 342993 | SLFSD | 367740 | NRFHA |
| 236273 | RGTAV | 302301 | EEDDK | 343000 | VMFSA | 367745 | EPTFP |
| 236291 | ETEAD | 302318 | LFSEK | 343001 | QNSDH | 367746 | LRVCW |
| 236495 | WGYFA | 302319 | VHEED | 343002 | FREEL | 367747 | VLLRL |
| 236671 | EAARL | 302321 | FMFLC | 343009 | GPGTA | 367748 | AQEKM |
| 236709 | GPGNK | 302324 | LDTTI | 343021 | LLTKK | 367751 | NLPVP |
| 236826 | NCRYG | 302327 | VGSDA | 343022 | GYSLT | 367752 | PLLAT |
| 236850 | KLNTQ | 302337 | DWWER | 343023 | IIMFI | 367755 | LTKHG |
| 236877 | HIRGF | 302355 | IQRRD | 343027 | DCGLF | 367757 | TEEGS |
| 236886 | KMGLH | 302383 | AQGTL | 343033 | YEKND | 367766 | YLELK |
| 236900 | AGEDA | 302386 | PTQEH | 343037 | EATAV | 367770 | DSKKN |
| 236925 | LPTKA | 302393 | NKLKL | 343040 | DDDDE | 367775 | VLSHC |
| 236938 | KATAE | 302397 | KETYY | 343052 | EDLEQ | 367778 | SGLYP |
| 236957 | AFNKI | 302413 | TEQVE | 343054 | FTEME | 367787 | LVAPC |
| 236959 | RLFHH | 302422 | QRKNM | 343062 | VIRKG | 367792 | PPAPR |
| 236976 | QGVLH | 302437 | TPSEV | 343064 | PRRRR | 367794 | QTCNT |
| 236979 | YRSHL | 302441 | VSMST | 343074 | SPLTK | 367795 | HPRKD |
| 236980 | VQSTQ | 302446 | TFLIP | 343081 | MKHDV | 367797 | AELEP |
| 236996 | CHKSD | 302452 | KGAKL | 343084 | IPSTL | 367798 | GYRSE |
| 237014 | TNPKE | 302455 | KVHTR | 343087 | QERPM | 367799 | RHPPH |
| 237172 | GGGKG | 302456 | PASSP | 343089 | PGPRA | 367802 | PRMGF |
| 237177 | VTTQF | 302468 | MAHIR | 343103 | ADTPD | 367806 | TGPCW |
| 237186 | AFLKM | 302476 | AIVEP | 343109 | LFGEK | 367807 | PGSQG |
| 237194 | EPKPS | 302485 | QVVGS | 343115 | HPGYR | 367815 | APKAS |
| 237201 | SEWNE | 302486 | HAAGV | 343116 | KRIHT | 367817 | EDTHL |
| 237247 | YLADN | 302490 | WLKKL | 343118 | EPIDV | 367825 | QVLLV |
| 237253 | APGYT | 302501 | MEDGM | 343127 | STVTL | 367828 | TLICS |
| 237261 | QNEVT | 302517 | TGFLS | 343129 | SNEAP | 367830 | TEESV |
| 237264 | RKEIL | 302521 | WWYHS | 343140 | QPAAQ | 367832 | QTPKL |
| 237275 | ASRAQ | 302530 | KRSRK | 343144 | PAVTL | 367835 | EYNAL |
| 237281 | LKPVL | 302537 | FWGKT | 343155 | QLISV | 367844 | AITPG |
| 237283 | ECQKS | 302543 | FRWFQ | 343158 | EDGED | 367845 | KLVHS |
| 237289 | KQMYG | 302548 | DLTTL | 343163 | EWHYL | 367847 | GFDAQ |
| 237316 | GTTAS | 302552 | NYISS | 343169 | TVRGG | 367848 | AAYAM |
| 237353 | GSSYC | 302562 | QGRSL | 343171 | KHKVT | 367851 | NKENF |
| 237380 | PLKPT | 302564 | LFSRK | 343172 | EQANQ | 367859 | VLTGP |
| 237455 | NDVPS | 302569 | KKFSM | 343190 | GFQQS | 367872 | LKIWN |
| 237500 | KDKDD | 302578 | EAVAH | 343199 | KLNPS | 367878 | DESET |
| 237512 | PIPSF | 302579 | AKWIP | 343204 | EALLK | 367879 | DLCSA |
| 237527 | RNSQG | 302586 | ALPPV | 343212 | RQGSS | 367882 | FSVFV |
| 237530 | KRTAH | 302599 | KWFWN | 343221 | GIAQV | 367891 | SVRIF |
| 237536 | LPPSE | 302603 | SCTSK | 343223 | PERKD | 367893 | PGATS |
| 237538 | RTKSN | 302606 | VSLSM | 343228 | SLRVA | 367894 | VTTIK |
| 237596 | SNVHV | 302620 | PKKKL | 343234 | DFLRW | 367900 | FSLKF |
| 237612 | LKKYS | 302621 | VAKSQ | 343242 | HRSLL | 367913 | ASEED |
| 237642 | WWGIH | 302625 | VERID | 343244 | MMISM | 367915 | KEYFF |
| 237654 | PVSVM | 302629 | NRTLS | 343246 | LTTSH | 367916 | RSMEY |
| 237694 | LSPAG | 302630 | TCTKA | 343248 | IEICV | 367922 | LVKNQ |
| 237696 | ELSNF | 302639 | GKKSQ | 343249 | VETLQ | 367923 | FQLPI |
| 237724 | SLPSL | 302640 | DSDSN | 343256 | IISRN | 367926 | ALTFK |
| 237763 | VLQMV | 302647 | SANKS | 343258 | GQRNH | 367928 | ERTNL |
| 237837 | FAKFI | 302648 | ECACV | 343271 | SSWSL | 367930 | GSSQG |
| 237841 | CYFSE | 302649 | KGFQQ | 343273 | ETLET | 367934 | YENDK |
| 237853 | AESWS | 302655 | GWSGS | 343274 | AKLYF | 367940 | AKAES |
| 237858 | IGALQ | 302656 | GGKFS | 343279 | DRKDP | 367950 | PLLLT |
| 237889 | DKKHH | 302657 | RSYTR | 343282 | YGFGG | 367952 | NRHHT |
| 238018 | PPPFP | 302665 | GKKGK | 343290 | NYTHP | 367957 | IGEKP |
| 238044 | NYDDY | 302667 | LFVTV | 343301 | MPVLC | 367959 | KVSCV |
| 238081 | EGAEN | 302676 | QVVTD | 343312 | PVGGL | 367963 | EEEEK |
| 238112 | LTPVH | 302684 | DPALA | 343313 | PQPTD | 367965 | FPPTT |
| 238138 | LGEET | 302690 | CCNTS | 343314 | STRRR | 367967 | LLRIF |
| 238146 | LEDDC | 302707 | ELQAK | 343318 | DCPPV | 367972 | SILCR |
| 238156 | TDRTV | 302716 | MTTSF | 343325 | VAGGC | 367976 | HFLGA |
| 238256 | IDWLG | 302719 | PHSKK | 343328 | IVHTL | 367983 | MSGST |
| 238341 | NASAR | 302724 | LSALC | 343331 | TPKNT | 367985 | RQLMT |
| 238349 | RYSTP | 302728 | NSLFT | 343339 | GLAQL | 367992 | KYLNA |
| 238379 | STPGL | 302741 | KQLYK | 343343 | MATEE | 367993 | REGFL |
| 238477 | TKLLV | 302745 | IQRRD | 343347 | LYEVM | 367995 | PYVSA |
| 238497 | FGQEG | 302756 | GGRGL | 343348 | FGTTV | 367996 | WERLE |
| 238508 | RLCSP | 302768 | RKRKQ | 343362 | LGGVS | 368005 | VSPAN |
| 238522 | DQEVN | 302770 | AAGEA | 343364 | TDSEH | 368007 | CLFFL |
| 238558 | LDSDS | 302777 | AQGPK | 343366 | AALKV | 368014 | LYGLE |
| 238576 | AFDIR | 302783 | SARAG | 343375 | KTFWS | 368017 | ESKNH |
| 238607 | AVPRR | 302790 | GTVKL | 343376 | TRKPP | 368025 | EEEQA |
| 238609 | EKHEE | 302801 | KPQLN | 343377 | QVYNV | 368026 | VPDPF |
| 238616 | CRPSL | 302805 | GRKEH | 343392 | GTQSH | 368030 | EPSPS |
| 238618 | FQIVK | 302811 | KVDSV | 343395 | DPIAV | 368035 | LLEEL |
| 238628 | QARHT | 302812 | NLLEI | 343398 | LQRSP | 368036 | LLEEL |
| 238633 | IVSHL | 302814 | SMSFT | 343412 | ERTAQ | 368037 | LLEEL |
| 238638 | SFRPM | 302830 | KVYIM | 343418 | DADGD | 368039 | DEAKL |
| 238647 | KERDP | 302833 | EVGPD | 343428 | NSEML | 368041 | LYELL |
| 238651 | IPSKV | 302836 | DKEQS | 343430 | IQAGL | 368045 | FLDIP |
| 238667 | SEDHL | 302843 | LRRGG | 343435 | FHLVR | 368046 | SRMEK |
| 238682 | SCKCS | 302846 | SEKCI | 343442 | KTADD | 368047 | AQMQL |
| 238686 | KSNWR | 302851 | VCTKV | 343443 | EYGKT | 368050 | PWGQR |
| 238688 | EKKDF | 302855 | SKDSS | 343445 | RFSSP | 368051 | LGFKI |
| 238699 | TGRLA | 302863 | KICGP | 343456 | ESGVQ | 368055 | GQVSV |
| 238714 | LTLNR | 302867 | IPSFH | 343458 | DKKEE | 368060 | SANIY |
| 238721 | LELPQ | 302870 | DVTFL | 343459 | IPKKK | 368065 | RSPLA |
| 238738 | CCLIT | 302874 | EGDAI | 343463 | FVHYH | 368066 | AVSKI |
| 238747 | PVPSV | 302886 | NEAGD | 343464 | HPPVL | 368070 | IESWP |
| 238788 | LISQQ | 302892 | NSVKI | 343469 | TWSCV | 368074 | CVYYW |
| 238789 | FETFL | 302895 | EQILK | 343477 | VWRPY | 368076 | LKKKT |
| 238831 | LYTGV | 302896 | DEEED | 343479 | CSIIV | 368077 | PVSSL |
| 238855 | EKADG | 302898 | AQMAS | 343488 | KVVSS | 368079 | NADER |
| 238875 | ITKLG | 302913 | FLQIS | 343490 | LRRQS | 368082 | LHKFY |
| 238892 | KQTSV | 302916 | GRAHP | 343493 | KAAAM | 368084 | ILPIG |
| 238918 | FQTEV | 302917 | PGYPV | 343496 | EQGRR | 368086 | FKEEL |
| 238936 | DVKMV | 302918 | LKSLP | 343497 | PVMQA | 368089 | CPVIT |
| 238971 | LSKKH | 302924 | APAGP | 343498 | VVSCP | 368098 | LCAWV |
| 238983 | SEDKK | 302935 | AAGDS | 343507 | PPVRV | 368102 | PYLRG |
| 238994 | LASYR | 302936 | QNPCE | 343519 | FPPGF | 368104 | GCGCR |
| 239032 | VSVVI | 302938 | IMPAN | 343521 | TVLSQ | 368105 | DQGVH |
| 239125 | GRARA | 302943 | TEDLS | 343526 | FYKKL | 368107 | DQGVH |
| 239138 | QLMLQ | 302961 | EMDID | 343545 | VVLGT | 368109 | DAMRE |
| 239144 | KGDKK | 302965 | ETSHM | 343550 | CHPDT | 368110 | DQGVH |
| 239151 | SAFQP | 302967 | SDVEI | 343552 | VKVDL | 368111 | DQGVH |
| 239165 | EEEEE | 302968 | QRWCP | 343557 | KKHKE | 368112 | DQGVH |
| 239223 | TSPSC | 302978 | QEKSV | 343560 | HFAFL | 368114 | GKAWG |
| 239231 | LPNFS | 302994 | HTSPS | 343563 | SRHRL | 368119 | TATIA |
| 239243 | MYHLS | 303015 | SPKSK | 343570 | HLWKI | 368124 | HSGSK |
| 239316 | VKLCT | 303019 | VIGRL | 343577 | VPKKP | 368125 | SSKLN |
| 239347 | LRRKD | 303028 | RSQIV | 343581 | IHNVT | 368126 | KPDSQ |
| 239367 | NGMYL | 303034 | VRDTM | 343617 | EEKLN | 368127 | EHHLS |
| 239440 | SQPLT | 303042 | KKGAS | 343619 | RAKDE | 368131 | TFIIY |
| 239444 | SLQLK | 303057 | EIADW | 343625 | VAIVA | 368132 | KTSKC |
| 239446 | GFNIQ | 303058 | SNASN | 343633 | KQRKK | 368135 | LQTVS |
| 239449 | GLNIQ | 303061 | RQPQV | 343635 | VPDDE | 368136 | LYKYV |
| 239450 | NNLGF | 303070 | EKCDR | 343636 | IPGEL | 368138 | DQDED |
| 239451 | QLEAY | 303071 | SHEQH | 343645 | FRSEL | 368140 | QVIKD |
| 239468 | PQFIS | 303076 | RQKFL | 343656 | IEPRV | 368144 | CASLS |
| 239587 | NKQHS | 303077 | IGIKL | 343657 | GIFRM | 368150 | PRLCH |
| 239597 | QKCIH | 303088 | DHKHR | 343665 | PYVKT | 368154 | GWIIQ |
| 239666 | ERTVH | 303096 | KKVFS | 343674 | GMSED | 368155 | DSPSQ |
| 239690 | VNTEN | 303108 | MNGDF | 343675 | TKLPG | 368164 | QLQTM |
| 239730 | CVEFH | 303111 | WKHIL | 343676 | KKQYV | 368165 | RPDGR |
| 239761 | EHSVI | 303129 | MPFIQ | 343677 | RRDLR | 368166 | FVDIM |
| 239830 | ELRLC | 303136 | PERSD | 343686 | LVSIT | 368173 | EIQLS |
| 239849 | VRDID | 303145 | KKLIE | 343690 | ITSLS | 368180 | DLNEM |
| 239854 | HYMSC | 303147 | KKLKY | 343695 | FSLSS | 368184 | IVFTI |
| 239882 | EPNSF | 303148 | THPWE | 343701 | EGFQL | 368186 | EKDEL |
| 239891 | TRKMN | 303149 | KGIGL | 343706 | GYSAP | 368189 | SSILP |
| 239893 | SMKPK | 303151 | VKVDP | 343709 | APQES | 368190 | RGHLV |
| 239906 | LIEEN | 303153 | NVKGP | 343716 | AEEAA | 368192 | CCPFP |
| 239926 | ESEEL | 303158 | QEDQL | 343718 | PDRMQ | 368199 | ACHNI |
| 239938 | TIEIC | 303163 | ECYGF | 343720 | GRDAL | 368207 | RVKET |
| 239940 | RDSGF | 303167 | MIYKN | 343739 | MDTGR | 368208 | RSCIF |
| 239944 | IRMGM | 303170 | VEESH | 343741 | WTPYM | 368218 | CNFLT |
| 240050 | LNVEE | 303178 | QRWCP | 343742 | GCYIS | 368219 | PFLKF |
| 240055 | QIQFS | 303182 | NIQLF | 343744 | PGSRE | 368231 | ISGIP |
| 240079 | SSFSD | 303191 | KRKRF | 343745 | QVPNS | 368237 | RLKTD |
| 240093 | DGTSA | 303192 | SKITL | 343746 | GSEQL | 368238 | VSNRL |
| 240100 | TSPSC | 303195 | ITSSS | 343747 | LVQHQ | 368241 | KKPKK |
| 240123 | YVAPV | 303204 | QARGV | 343748 | VQESV | 368244 | FSQRG |
| 240132 | LAGMI | 303206 | CKFKN | 343749 | VIFPQ | 368245 | SNVNP |
| 240185 | SGWGM | 303208 | SQELQ | 343757 | LWTLS | 368251 | APLWR |
| 240189 | LHLCC | 303212 | HTLDS | 343765 | SDSER | 368252 | EAKSD |
| 240285 | AKNGI | 303222 | LKMLI | 343770 | VILQF | 368253 | LCTKI |
| 240304 | DTQSN | 303231 | YLNAS | 343782 | SWGVS | 368264 | SSHPM |
| 240306 | SPQMM | 303234 | YSEYV | 343785 | QGKPS | 368266 | SSQPM |
| 240316 | STEPA | 303239 | SSPQT | 343799 | VRKQA | 368267 | HAPSA |
| 240327 | RLKQS | 303242 | KFAES | 343802 | KPLQP | 368270 | LLKIQ |
| 240328 | RESPK | 303246 | FVSTF | 343807 | KQEVI | 368273 | EKAGV |
| 240333 | KKTSH | 303248 | LNSYS | 343811 | GSKKD | 368280 | ALACP |
| 240335 | NWTDG | 303249 | EIDMA | 343818 | SRPGS | 368281 | LSLHH |
| 240343 | LFKSG | 303252 | LQPLF | 343819 | KFQVL | 368282 | ALACL |
| 240349 | MELTM | 303257 | KRGCD | 343820 | IRAER | 368283 | LSLHH |
| 240361 | SDLSD | 303276 | LDRII | 343841 | VKPNV | 368284 | ALACP |
| 240364 | SSLMV | 303279 | VVSGK | 343847 | QPAEP | 368286 | LSLHH |
| 240423 | VRQGD | 303282 | AWISP | 343864 | PSPSS | 368287 | ALACP |
| 240488 | FDYID | 303300 | VHEED | 343871 | QKKAV | 368288 | LSLHH |
| 240499 | KKIHT | 303315 | KGHAF | 343877 | HKKET | 368289 | HCSGH |
| 240615 | IACMI | 303316 | PITAG | 343880 | QDSDS | 368290 | ALACP |
| 240617 | KLDIK | 303325 | VDEYS | 343888 | LPETT | 368291 | LSLHH |
| 240618 | MQRTV | 303349 | SSPQT | 343890 | LLDLP | 368292 | KEMGN |
| 240619 | NLRVT | 303356 | GFWAC | 343891 | LQKLG | 368293 | ALACP |
| 240651 | EDYFD | 303366 | NKRFY | 343894 | VALIQ | 368294 | ALACP |
| 240652 | NYLPL | 303373 | KAKGK | 343899 | PEFVV | 368295 | LSLHH |
| 240662 | NTSES | 303394 | PVGTK | 343901 | QNDGD | 368296 | SASLS |
| 240687 | QHKQI | 303398 | KTSKK | 343924 | QSVVI | 368297 | ALACP |
| 240691 | KPFVP | 303408 | YTSSK | 343925 | NPQSQ | 368298 | LGLPS |
| 240700 | MWQES | 303423 | TNVQQ | 343930 | SSTSL | 368300 | HRRGR |
| 240727 | PEAQR | 303424 | MESSL | 343939 | LSFKQ | 368304 | SSQLQ |
| 240851 | GPGYR | 303434 | GRLVF | 343943 | TEPNS | 368305 | NVENA |
| 240922 | QKTDN | 303437 | TSVRK | 343948 | EGDVL | 368312 | GAETQ |
| 240986 | DDLTV | 303452 | EPLEE | 343966 | KRRRY | 368315 | FSQPY |
| 241001 | WPRLQ | 303458 | QGCLF | 343989 | YCVLQ | 368317 | MNSFV |
| 241014 | DIYLE | 303468 | QYLGS | 343990 | IPRLP | 368318 | FGSHG |
| 241041 | LPGHP | 303469 | FKKVT | 343995 | LVGFC | 368319 | GALFF |
| 241051 | EHFGD | 303476 | REDEG | 343996 | DADDH | 368320 | LRLRA |
| 241052 | EKANL | 303482 | SDVDS | 344014 | KGHPL | 368330 | STLGG |
| 241071 | PAPET | 303486 | RRQRP | 344023 | SLFDL | 368332 | GKEVC |
| 241124 | TGFPS | 303488 | ASLLK | 344026 | SSEGK | 368340 | PGGRR |
| 241125 | EDLAI | 303490 | SNSKK | 344029 | GLGAS | 368341 | TDRMQ |
| 241256 | SSINT | 303500 | LQKIK | 344030 | SNSAV | 368342 | AISAP |
| 241274 | TTYRF | 303507 | FSTEV | 344034 | YLADC | 368343 | IKVDL |
| 241279 | PKSFP | 303508 | MLGCS | 344040 | KGFTQ | 368347 | NLTNA |
| 241305 | RRQRG | 303511 | KFAAC | 344041 | MMPDN | 368351 | PMGPT |
| 241393 | SFHSS | 303515 | GSALA | 344042 | ETVPV | 368353 | RNFLI |
| 241416 | KESSL | 303518 | SSPQS | 344058 | FYLTP | 368357 | EAFCS |
| 241436 | DIFFK | 303522 | SNVLS | 344071 | NTRWK | 368358 | CAVKS |
| 241463 | PSALG | 303525 | LQLIL | 344077 | PQRLY | 368361 | TTEDE |
| 241502 | FVTVG | 303532 | CCKKP | 344083 | VPKKP | 368362 | IYEKV |
| 241527 | DDEEE | 303540 | KAKGK | 344087 | DIEAR | 368363 | EGNNK |
| 241600 | MSHSL | 303549 | MQSYG | 344101 | SLAHL | 368364 | WVKFW |
| 241651 | ETMPN | 303550 | PDRQR | 344114 | VYPIV | 368378 | MSLDD |
| 241802 | HQGLL | 303552 | AYNGM | 344115 | EELLY | 368381 | YFAGA |
| 241808 | TCRRH | 303554 | AVESQ | 344125 | LVCNT | 368386 | TMKQN |
| 242044 | SLQKR | 303570 | QVSEE | 344129 | SSSDE | 368388 | FYTSA |
| 242057 | SSGFL | 303575 | DCHAL | 344133 | GSIPT | 368389 | FLYRI |
| 242059 | IKFFK | 303578 | ALALY | 344140 | CVAHS | 368391 | TQESL |
| 242066 | EGYVY | 303584 | KFTKS | 344144 | ELPSV | 368396 | RQIWI |
| 242067 | QGVSE | 303585 | DDETF | 344149 | KSSGH | 368397 | MLTGS |
| 242104 | EMVHS | 303591 | RPPRP | 344151 | HGGNH | 368402 | LHFIK |
| 242108 | IKHER | 303599 | HPNYN | 344162 | MWESL | 368405 | SQEEQ |
| 242109 | TFNIC | 303616 | VGPLC | 344166 | QCYSN | 368412 | TIFTY |
| 242110 | TITKY | 303623 | KESVM | 344173 | SNHSI | 368433 | ATSYI |
| 242140 | QASAR | 303624 | QDAYF | 344174 | FSPLK | 368438 | DEEGS |
| 242152 | DPAMW | 303627 | SSEYV | 344182 | RIKQS | 368439 | DEDEK |
| 242158 | TLSTD | 303634 | DDGLP | 344192 | VHHVA | 368443 | NRIQL |
| 242159 | EDEEE | 303659 | LNSTF | 344193 | VEDST | 368449 | FLTTD |
| 242208 | ECGCS | 303666 | TRPPD | 344194 | SSVNK | 368450 | KTELV |
| 242209 | KHDEL | 303686 | PETRT | 344203 | YVSHL | 368456 | PASAS |
| 242210 | LQKIL | 303706 | RLKKL | 344210 | EASPA | 368459 | KSKDV |
| 242249 | TDTLL | 303709 | KRYAT | 344215 | RQYNY | 368460 | YKAKQ |
| 242257 | GTVKQ | 303712 | DSSRY | 344217 | TAVVI | 368461 | TCQTQ |
| 242261 | MSASH | 303713 | LRTSV | 344218 | IKVTN | 368462 | WVGDA |
| 242275 | LLKVI | 303727 | SKDFA | 344220 | DAAVQ | 368465 | VCSLI |
| 242285 | APLVH | 303736 | VHEED | 344223 | KTPSV | 368466 | GPSAP |
| 242310 | DGHGK | 303740 | KHLRC | 344226 | QPQAL | 368468 | YRLWI |
| 242315 | LKPPG | 303742 | HFIDR | 344235 | LGPEA | 368472 | GTVPS |
| 242317 | VKIKT | 303754 | AKMFA | 344240 | CWRYR | 368474 | PKTGN |
| 242338 | SSLLI | 303766 | PEVVM | 344241 | RVQWG | 368483 | FRMGT |
| 242351 | ACVIS | 303775 | CIRLK | 344242 | ITIVG | 368485 | SADHD |
| 242365 | QNFHS | 303777 | QLGGL | 344244 | PVADL | 368488 | KPAKV |
| 242375 | FHDEY | 303779 | LDLGS | 344254 | RNRCR | 368490 | SLKTC |
| 242462 | FSDFL | 303788 | PQELL | 344259 | KRPVD | 368496 | REGIP |
| 242465 | EDFML | 303800 | APEQQ | 344260 | AVDER | 368502 | IEAAF |
| 242475 | LCIVQ | 303802 | TGSLD | 344270 | RLWQQ | 368503 | LKPDD |
| 242480 | RTRTP | 303812 | EAKSD | 344276 | AELKN | 368508 | YFLME |
| 242505 | TRQGP | 303817 | EVQPH | 344277 | KTPSQ | 368511 | LHSIF |
| 242576 | DWKEL | 303818 | DSPIK | 344285 | GPHVL | 368514 | PPASC |
| 242577 | LFKSG | 303820 | KCHNT | 344298 | SLKFS | 368515 | QQADE |
| 242591 | DRLIL | 303822 | FRFLE | 344307 | RAYGW | 368516 | PRVKD |
| 242592 | RSYRS | 303830 | RSNPS | 344314 | LDDSW | 368517 | SHEPV |
| 242607 | LGEVN | 303834 | FMTLG | 344322 | SQATG | 368523 | TTTKH |
| 242630 | DWVYE | 303839 | GSAEI | 344327 | CRRIS | 368524 | PSACL |
| 242719 | SYETN | 303848 | PGRGS | 344331 | QEAEK | 368528 | SPGTK |
| 242728 | GKEAP | 303850 | LRRTR | 344336 | QAQRS | 368529 | YFFIK |
| 242729 | PQQKI | 303862 | RNCIP | 344343 | SPASD | 368530 | IGTER |
| 242753 | WILNN | 303864 | RKLFP | 344348 | FAAAL | 368532 | PNLVR |
| 242770 | LLFSL | 303887 | RRHRD | 344352 | GTLQS | 368533 | SGAFV |
| 242776 | IEQSR | 303889 | MSVIP | 344353 | NESAA | 368535 | RAEPL |
| 242784 | RPLVK | 303890 | VPETL | 344354 | SGAVL | 368538 | TLAKI |
| 242786 | SESGI | 303899 | RDTWE | 344361 | TCSLL | 368544 | SQPRN |
| 242796 | EDGAG | 303909 | FSTDV | 344364 | LTLTL | 368547 | GLLPT |
| 242804 | WRDTL | 303915 | MSVTI | 344374 | RARLS | 368552 | NETVS |
| 242810 | KCFKL | 303918 | LKHSS | 344387 | LRRLC | 368558 | IIFLL |
| 242819 | SRGRA | 303920 | HWCFC | 344392 | SDNDF | 368565 | SRAKK |
| 242827 | VKKFQ | 303928 | SRTHL | 344393 | GAPRF | 368568 | RRRGP |
| 242839 | EEQYI | 303939 | FIPIN | 344396 | SQNVQ | 368577 | TGISA |
| 242848 | ELCVS | 303942 | TEALD | 344398 | NNKNT | 368580 | IWCET |
| 242872 | EAFHQ | 303960 | VRADK | 344402 | PSLTH | 368589 | MSLYC |
| 242994 | TVFTE | 303963 | ILAAN | 344403 | HQDEH | 368591 | ENARP |
| 243045 | GINKY | 303968 | LELQT | 344411 | QGKHE | 368605 | YLVEP |
| 243052 | NGNLD | 303977 | IDIPE | 344419 | WREAS | 368607 | EDQAA |
| 243056 | HLHST | 303983 | FRSSR | 344424 | LLKEA | 368612 | MLSSV |
| 243077 | GDPLA | 303987 | AYRSL | 344426 | YSGPP | 368614 | ASTAF |
| 243082 | GNPLL | 303989 | EGKEL | 344430 | PLTDY | 368621 | SAGLW |
| 243103 | ALSFF | 303992 | AKKLE | 344431 | QALLT | 368629 | DRASS |
| 243108 | EKQKE | 303997 | ANMGF | 344446 | EGTIL | 368632 | VTAMG |
| 243152 | YQSHL | 303999 | MQVYE | 344453 | KRTFL | 368640 | CWRYR |
| 243167 | EKQSS | 304004 | LLNAS | 344456 | FDTDL | 368644 | RISNV |
| 243189 | LWIPI | 304006 | QTTIA | 344458 | KQIMN | 368646 | FDKLN |
| 243213 | FFCDT | 304017 | KLLKE | 344468 | EEFYA | 368648 | GQPRP |
| 243222 | LVAPM | 304032 | KQFHE | 344479 | DTLPF | 368660 | CWRYR |
| 243253 | GALLF | 304033 | IRRMN | 344488 | VHFLT | 368661 | ALKYL |
| 243286 | DIPYL | 304046 | FCEKM | 344489 | VHPSP | 368664 | TLASM |
| 243297 | WQQRK | 304051 | NDDTD | 344503 | YQSLR | 368665 | RAHLS |
| 243298 | GSSCC | 304060 | SFKTM | 344504 | AGKKK | 368666 | QITTV |
| 243300 | SNYDV | 304065 | WAICP | 344519 | KLYVD | 368667 | FYFLS |
| 243314 | EQEGV | 304071 | RTDSS | 344524 | GAGEQ | 368675 | VIIDI |
| 243325 | SSSCC | 304072 | MRKRM | 344536 | LEENS | 368677 | GVCPQ |
| 243326 | HENSI | 304077 | FLETK | 344537 | PQPAP | 368678 | PCPTP |
| 243344 | ASLRP | 304078 | IHKNK | 344545 | SQDGA | 368679 | YLCES |
| 243346 | AYFEE | 304096 | TSANL | 344549 | EQSPR | 368682 | SCRLW |
| 243347 | RFSHL | 304102 | RGLFF | 344551 | LKLHM | 368683 | NRAHW |
| 243349 | EDCKA | 304108 | LIPIN | 344552 | TPLQM | 368684 | TPATI |
| 243389 | TCAFI | 304111 | VKSER | 344562 | VDGTD | 368686 | PVLNL |
| 243440 | GCLPR | 304127 | TEFLN | 344563 | TEDLD | 368691 | ALTVH |
| 243451 | KLESE | 304133 | AMENM | 344568 | RIHTG | 368696 | EEKVE |
| 243457 | RESEI | 304139 | GRSRY | 344572 | RLYHD | 368699 | PGGRS |
| 243501 | EKTDL | 304146 | TGSAF | 344577 | PCHTH | 368716 | LANEI |
| 243563 | SFAKK | 304147 | CIDVR | 344579 | RPPEL | 368719 | LVQWI |
| 243578 | YGVEC | 304151 | VRLFT | 344582 | LSEES | 368723 | VKNIR |
| 243611 | KAKLL | 304169 | VDRPV | 344591 | HILSD | 368727 | WSVRV |
| 243639 | SGEKP | 304185 | NGNAQ | 344609 | MLAPH | 368730 | ENRLS |
| 243643 | PYKRN | 304188 | IINKN | 344616 | FKYVM | 368732 | ADRHL |
| 243644 | VTENP | 304192 | LGAEV | 344624 | QLQPP | 368736 | LISIR |
| 243662 | GCAKS | 304198 | RPEKR | 344626 | GQRQL | 368737 | ILATL |
| 243673 | IVTMS | 304223 | FNNPT | 344627 | YNDKP | 368741 | HLASE |
| 243706 | VSLED | 304226 | QYPYM | 344635 | PEMVP | 368743 | LYEVM |
| 243776 | QGNST | 304229 | HWPPG | 344637 | LQYFK | 368746 | LYEVM |
| 243786 | HCACI | 304236 | ARGFA | 344648 | PFKEQ | 368747 | ICSAR |
| 243806 | PSGFK | 304250 | MNLDR | 344655 | GSSGE | 368748 | FLIVG |
| 243810 | VESEK | 304257 | SILFN | 344658 | QSGGD | 368749 | RRRWT |
| 243878 | VKMDD | 304283 | KCTVF | 344659 | NQYIK | 368753 | NRNKQ |
| 243896 | AQGQQ | 304286 | EGTEV | 344666 | FFEEL | 368754 | KQANL |
| 243903 | AGEQA | 304290 | PDPFP | 344668 | TERNS | 368756 | SPPST |
| 243914 | NTMDK | 304292 | SASRS | 344674 | PGPGA | 368758 | DHALA |
| 243918 | PKSNV | 304308 | NDKKT | 344683 | MGTSL | 368759 | EMATF |
| 243919 | DDDDE | 304311 | CKFTD | 344689 | RFTPY | 368760 | SPPGT |
| 243924 | CFVPQ | 304321 | EEVKS | 344699 | SEEKA | 368761 | NSGQW |
| 243938 | EIPVP | 304327 | DDAED | 344729 | RNHTL | 368766 | RETDI |
| 243964 | ITIYS | 304331 | LTVTP | 344732 | PGHVD | 368767 | SLPAI |
| 243967 | MHADR | 304336 | LQRAA | 344737 | YCVLQ | 368769 | KIHPV |
| 243997 | KVKKE | 304350 | EDLYA | 344742 | ITDLR | 368770 | EITAH |
| 244007 | GDNRL | 304353 | RVTPK | 344758 | PQNIK | 368775 | VPAIS |
| 244020 | SSSRD | 304355 | SGSIR | 344759 | DLNEM | 368777 | LLPAA |
| 244040 | KRSCC | 304360 | VGPAL | 344777 | QKSYR | 368779 | DLEVN |
| 244043 | YRIRP | 304364 | GSDND | 344779 | RFDSQ | 368785 | DWSIS |
| 244047 | RAVSG | 304370 | IKLWD | 344781 | PSSKP | 368790 | LKSAK |
| 244050 | SGCPR | 304376 | SCCHH | 344782 | QHNLK | 368792 | SMETT |
| 244051 | TFPQY | 304401 | TCCVL | 344789 | DSPST | 368794 | LRLCT |
| 244061 | SIIDQ | 304408 | PVCFL | 344791 | VGKLD | 368797 | QQGSL |
| 244096 | FSYPE | 304410 | RQSQH | 344794 | DFHRA | 368798 | MVTAR |
| 244137 | FSGPK | 304414 | SMFQL | 344798 | DTFDS | 368799 | RVAPQ |
| 244174 | RSWTF | 304419 | QRKFP | 344801 | CPVSI | 368801 | RGDDQ |
| 244204 | SYTFS | 304422 | LISAL | 344811 | QHCSQ | 368805 | NNEDF |
| 244217 | ELEQA | 304429 | SEDKP | 344819 | QGDQQ | 368808 | PKAEQ |
| 244221 | PGEKY | 304440 | AFAEV | 344820 | QAPAC | 368809 | SVLAL |
| 244227 | LDFIA | 304447 | QAGAA | 344822 | KIRKK | 368810 | GRRIT |
| 244230 | HKLKL | 304467 | EPQVC | 344823 | TKSLE | 368813 | DLVLG |
| 244241 | LPRSV | 304495 | RRRWT | 344828 | KCGSL | 368814 | PQRPA |
| 244249 | VHLSY | 304500 | CKLAS | 344829 | EYLHL | 368818 | ALFAL |
| 244289 | CGGRH | 304501 | LGVGL | 344831 | RRQQY | 368824 | IETHV |
| 244314 | DSEEK | 304502 | VDLGS | 344832 | TEIAG | 368828 | WCICL |
| 244316 | GSSTR | 304507 | KGKRD | 344844 | LYATD | 368829 | ICEYH |
| 244321 | CTHSP | 304519 | LAWNC | 344847 | KSKEL | 368831 | AKAIA |
| 244333 | AGVLL | 304544 | RRLQD | 344851 | LSHCV | 368839 | GTTSS |
| 244336 | RVALI | 304553 | GRYFC | 344855 | GSSRR | 368856 | FDVQD |
| 244360 | VPAES | 304565 | SRRCC | 344860 | HVLYP | 368858 | LMTSD |
| 244426 | SERPL | 304583 | RLLNK | 344866 | CRSGE | 368859 | VDENC |
| 244458 | YVEAI | 304586 | KWRET | 344903 | FSAAS | 368868 | VTCEK |
| 244496 | PLSKE | 304590 | RTRVP | 344909 | HCCVQ | 368870 | SSVGL |
| 244513 | SQGAP | 304592 | SVREG | 344914 | KTEYT | 368872 | EESFV |
| 244519 | TEALY | 304593 | LKWTV | 344923 | SKDTL | 368876 | PFQLD |
| 244527 | QHTRL | 304595 | RPWLR | 344925 | GSLKP | 368880 | SWVSG |
| 244534 | APKKK | 304604 | AEKRQ | 344929 | DLKLG | 368881 | ISKLA |
| 244537 | QGRTL | 304642 | LLGGH | 344936 | CDIDI | 368884 | TSTAL |
| 244558 | TVYTL | 304643 | FKLIQ | 344937 | LSRNK | 368887 | IKTMQ |
| 244563 | ILTSA | 304656 | PGSSR | 344942 | YNGGQ | 368889 | NAQME |
| 244565 | LDEKL | 304668 | PLCIC | 344945 | NVLLK | 368891 | PSACL |
| 244571 | ALSQL | 304670 | HLQIY | 344961 | ESSEI | 368893 | IGTER |
| 244573 | APKKK | 304689 | KKHGM | 344967 | PDKPF | 368908 | KHYPY |
| 244576 | LHTKE | 304697 | LRGGC | 344976 | TSDIW | 368910 | IPAVP |
| 244601 | YTSAK | 304701 | QYSFY | 344984 | VKSRV | 368914 | YLEKL |
| 244620 | FSTYF | 304704 | FMDLK | 344989 | PLSHI | 368920 | PSFGS |
| 244623 | FLIKK | 304707 | RSYQY | 344996 | EEEPE | 368927 | DIDDI |
| 244625 | NIQWD | 304710 | RDMRN | 344998 | CPLGA | 368931 | IQRRV |
| 244636 | CGQLE | 304713 | EGHLQ | 345001 | RMDRL | 368933 | EKHRK |
| 244661 | RGERA | 304736 | TTKAE | 345014 | FPPGF | 368940 | EWHYL |
| 244669 | ADILK | 304743 | VSGQS | 345016 | IISRN | 368946 | KVLGL |
| 244709 | RSFVP | 304748 | KESKK | 345023 | DFGLC | 368948 | VGGGL |
| 244710 | RNCKS | 304750 | PEEIN | 345029 | ASEED | 368949 | SARSL |
| 244711 | SPAWK | 304762 | PKIHP | 345030 | GLAYS | 368951 | NETSL |
| 244728 | KGPNY | 304767 | GDTSL | 345031 | LCLKK | 368952 | LPNKA |
| 244741 | SKRKP | 304769 | PPLFF | 345040 | VAKDV | 368959 | EKKST |
| 244745 | LVFTY | 304782 | AQEDL | 345044 | EEAGQ | 368961 | SGRVE |
| 244751 | LHTHI | 304791 | MKKHR | 345055 | HVKLK | 368962 | RVGSC |
| 244759 | GRRPR | 304796 | LFSEK | 345064 | NCIIS | 368964 | LNTFC |
| 244763 | VGSDE | 304802 | GELWK | 345065 | KCVVS | 368965 | IFWKI |
| 244766 | TWLSF | 304803 | MPQAN | 345079 | LVHSE | 368966 | MLRCE |
| 244769 | SNVGK | 304807 | GAFSS | 345090 | LRPFF | 368976 | RPLPV |
| 244795 | HVTTV | 304811 | KGKND | 345096 | EKRLY | 368981 | LAQTV |
| 244799 | HEEWE | 304822 | TPPTA | 345102 | ALLWD | 368983 | FRLKK |
| 244820 | IRGMI | 304825 | SVTQA | 345107 | ESEGP | 368984 | KRAAN |
| 244869 | ELPQV | 304826 | RLQWE | 345125 | NEWVS | 368986 | SLNKQ |
| 244906 | GREGK | 304835 | RRRWT | 345133 | LESSP | 368987 | FSLNK |
| 244926 | KKCSV | 304843 | CCCKK | 345140 | DGGDM | 368989 | FYANQ |
| 244930 | NMKSN | 304846 | CDRSI | 345144 | GAFKN | 368990 | FFTST |
| 245046 | QTSIF | 304854 | SNLEK | 345147 | YGRTW | 368991 | SKHSF |
| 245074 | PQGRE | 304858 | GINKY | 345151 | SSEGK | 368996 | GQRQL |
| 245105 | PSHPR | 304875 | RTHNP | 345152 | KTKQP | 368999 | SKTLN |
| 245121 | EFESV | 304884 | GFSVS | 345153 | EMLLP | 369003 | VTTRL |
| 245157 | GTASS | 304891 | RKFFI | 345156 | SGKKA | 369014 | TRKFC |
| 245185 | CSCCA | 304895 | PEDRQ | 345161 | HSPDL | 369018 | SSSRI |
| 245206 | HQVTK | 304899 | KMWTS | 345162 | YLEHC | 369021 | RDHPG |
| 245222 | PGREP | 304903 | FDLYT | 345168 | KTVEE | 369034 | TCLTY |
| 245255 | RLYYL | 304908 | IAELK | 345179 | CPKLS | 369038 | YTNSH |
| 245304 | ACNIQ | 304915 | EGQFN | 345184 | TLAIH | 369042 | APGMN |
| 245312 | QPDEK | 304923 | FDLLS | 345192 | TEDDT | 369046 | KDGKC |
| 245323 | IYYKV | 304926 | LSGRS | 345193 | QLLDR | 369050 | MQLRS |
| 245407 | KSTAF | 304941 | CKAYS | 345195 | GSQPS | 369055 | IGTPS |
| 245414 | IPCAP | 304945 | PVDYS | 345200 | GRGQA | 369059 | CQLLN |
| 245441 | PPLRS | 304956 | VAALT | 345206 | ATVVS | 369061 | LGNKS |
| 245451 | GCGCR | 304965 | RCWQV | 345216 | QHSQW | 369062 | RFLGL |
| 245457 | KQADL | 304985 | EVGLP | 345217 | PRRRR | 369071 | EGRSQ |
| 245479 | QLTRP | 304997 | PENSS | 345223 | EFYVD | 369075 | LGHQS |
| 245503 | VISEE | 305005 | LAIGI | 345228 | QVSPH | 369076 | KLWKP |
| 245539 | HYRWW | 305011 | RVTFQ | 345229 | KTKDL | 369080 | ERKQR |
| 245541 | QWGNL | 305027 | QYNSL | 345230 | ELNCI | 369081 | AKDEL |
| 245543 | APRQR | 305039 | HEGDD | 345243 | EVKFQ | 369090 | AVHHE |
| 245544 | SLEGS | 305056 | CLLAG | 345259 | EIADE | 369091 | TEVTV |
| 245552 | AAQRE | 305059 | MKLSV | 345270 | DEAEV | 369095 | SRFLL |
| 245564 | DSLVD | 305069 | QPSRT | 345279 | GEARR | 369099 | RAFHC |
| 245615 | KLREE | 305071 | PADPE | 345280 | SCQLL | 369108 | KFSPY |
| 245649 | KLRAG | 305077 | WRKTL | 345281 | FLRRL | 369112 | SSDSD |
| 245663 | FAWLS | 305089 | YEERT | 345282 | LEKYA | 369119 | LSPAE |
| 245680 | EDGAS | 305096 | RKGPD | 345294 | CLACV | 369121 | GPVCC |
| 245796 | NRNQL | 305097 | ITMNP | 345317 | FALPL | 369123 | DPALF |
| 245810 | CGCGG | 305107 | IGILQ | 345328 | YVEKN | 369126 | NVKDN |
| 245811 | FSLKE | 305119 | CLVVR | 345333 | YGPKP | 369127 | QCQTS |
| 245812 | PPPAC | 305133 | PVKAK | 345340 | TPKKD | 369129 | SSIGH |
| 245816 | VPAST | 305135 | SLQAY | 345341 | ASPAH | 369130 | RQRSK |
| 245817 | SPRSE | 305151 | AGSRH | 345342 | VCMPV | 369131 | RPASP |
| 245903 | QWVRP | 305161 | RSPDS | 345344 | SYPTV | 369135 | EETDL |
| 245907 | FGCPN | 305182 | SQILS | 345359 | EDTHL | 369136 | YTAPC |
| 245908 | LEPDR | 305193 | APSLF | 345364 | FNIVS | 369139 | CSCCA |
| 245912 | GAFMV | 305198 | GLPEC | 345368 | KCHVM | 369144 | CSCCA |
| 245919 | SLLAL | 305200 | SSVIW | 345370 | LPESL | 369146 | CSCCA |
| 245923 | KAKAE | 305203 | DEKPF | 345374 | YPQQV | 369147 | KHWTQ |
| 245925 | QWRVV | 305204 | QTPES | 345393 | PASCS | 369150 | ARGKN |
| 245932 | KRGSP | 305207 | KRISS | 345395 | QPIWQ | 369154 | ISSVS |
| 245934 | AKGNS | 305221 | KGKRD | 345405 | EEAAR | 369157 | GSQKL |
| 245983 | SSNKV | 305223 | PSVSN | 345412 | RDRHH | 369159 | RNCIP |
| 246000 | EHDGT | 305230 | TMETE | 345420 | EEGGS | 369161 | DLKKQ |
| 246006 | PGTDC | 305244 | ALPSA | 345431 | PAMRF | 369164 | PVWPA |
| 246012 | KCEDA | 305255 | VWPTN | 345433 | PGLSY | 369165 | SLLLY |
| 246020 | GRNLR | 305259 | LKINK | 345436 | DAMLE | 369176 | PDQHH |
| 246024 | ADKGL | 305260 | LKIWN | 345445 | GIRFL | 369179 | LGQDL |
| 246027 | DDFYD | 305263 | QRVPF | 345449 | PGVLT | 369193 | THLKS |
| 246032 | VTCAA | 305270 | SQLLS | 345456 | VPDTW | 369198 | FIEQM |
| 246039 | PKRDS | 305286 | RARLS | 345464 | KVDWG | 369199 | LTWWL |
| 246041 | AAWPR | 305288 | STTRV | 345466 | IMQKN | 369210 | QFWAY |
| 246043 | EGTSV | 305297 | KGQPH | 345468 | HETIV | 369213 | AEMSK |
| 246062 | IEDKN | 305302 | GDLKY | 345469 | TLLTR | 369217 | EYIED |
| 246069 | EGCPV | 305343 | HATAM | 345470 | EYFFT | 369218 | LAEQV |
| 246070 | YKHMG | 305347 | PLDPP | 345471 | FSSAK | 369219 | GKWYP |
| 246071 | TYAKK | 305355 | PEVKS | 345477 | VYRGN | 369229 | IHACS |
| 246077 | LQLFP | 305372 | NDSLL | 345478 | EGNEH | 369238 | QSSSN |
| 246080 | RGPRR | 305373 | GHKHT | 345479 | RRKAL | 369239 | TQPWS |
| 246081 | DFFCE | 305403 | KLVYK | 345487 | RFGRR | 369241 | SLWGF |
| 246090 | CACFL | 305416 | VNSSS | 345491 | GFNIQ | 369250 | RNDLD |
| 246100 | PAAEG | 305422 | SSGHC | 345492 | QYTPL | 369251 | SLEKL |
| 246104 | AGPAS | 305424 | VVFSV | 345494 | KSGVW | 369252 | PEYYC |
| 246105 | EAEEQ | 305426 | KLACE | 345511 | WKSLQ | 369256 | SQYNV |
| 246108 | QAESF | 305430 | MRRLT | 345528 | INDAS | 369257 | DDLEA |
| 246115 | SVRSI | 305441 | GHGKF | 345530 | FDGVD | 369263 | LGKTD |
| 246117 | KAKTQ | 305442 | RSVNY | 345532 | ESTAC | 369268 | PPGPE |
| 246139 | FPSSC | 305449 | RNPGV | 345555 | GASSG | 369274 | GCQMQ |
| 246149 | QSPSM | 305459 | PSVGP | 345563 | AASCL | 369277 | EENQY |
| 246151 | THFIS | 305464 | GPSLD | 345571 | TPLDF | 369279 | FKTDH |
| 246166 | EPATD | 305465 | VKEEL | 345575 | ASLHG | 369280 | AVSLF |
| 246174 | LILKL | 305469 | KISSL | 345599 | TAIRS | 369291 | SSKGA |
| 246186 | VQEWV | 305480 | FKRGK | 345621 | WMGGQ | 369292 | DIEIV |
| 246190 | IMNNN | 305483 | LLPQQ | 345629 | TKDKA | 369293 | SRRTI |
| 246194 | DGALQ | 305494 | NVEGI | 345633 | QGMSQ | 369297 | CTAAG |
| 246222 | IGGKG | 305502 | GMIPS | 345637 | SGLTK | 369299 | CPPSS |
| 246229 | HQAFQ | 305503 | RITFD | 345652 | LGARV | 369312 | CSHHL |
| 246337 | LLRQN | 305520 | SWGSS | 345656 | GKFIL | 369313 | AVCHR |
| 246421 | MLPAS | 305524 | WASLQ | 345659 | ASFRA | 369316 | FVALG |
| 246505 | LSTVC | 305529 | WKQKT | 345667 | RSVVI | 369317 | KSYKH |
| 246515 | CNSEL | 305530 | RFHRP | 345681 | VTAMG | 369318 | RCLVM |
| 246529 | EPVGP | 305533 | GYPFK | 345689 | QCCSN | 369320 | VSKCA |
| 246532 | GVCWA | 305538 | YSSLT | 345691 | RAHLS | 369321 | CLLPH |
| 246533 | LTMYS | 305556 | GMGCS | 345702 | IIRVS | 369322 | AVWAP |
| 246535 | ELLFK | 305590 | SPYPY | 345703 | CDVSS | 369323 | CSEER |
| 246538 | ACAEN | 305595 | AHLKC | 345708 | PDSLS | 369325 | ALTGK |
| 246548 | VIALD | 305596 | ETKSI | 345716 | EKDDK | 369335 | LFLVF |
| 246549 | DFTTE | 305603 | AAWFT | 345719 | YYYDD | 369336 | IAKLS |
| 246551 | MPGRG | 305613 | GRVHF | 345721 | TTNED | 369339 | VLMTF |
| 246553 | GKSQK | 305627 | MRHPK | 345729 | LFFFG | 369341 | IKSSS |
| 246635 | DVRRP | 305631 | AGGLF | 345731 | AKEKL | 369344 | CESAF |
| 246646 | TGSRF | 305632 | LGTDN | 345752 | VQTVV | 369345 | RQAPN |
| 246657 | TTFSP | 305638 | RVLHA | 345765 | HGARR | 369346 | NPRAV |
| 246662 | KSSHS | 305640 | KWKGK | 345767 | VQKEN | 369349 | SCRKC |
| 246672 | RVDAQ | 305647 | SGPGN | 345771 | GEAAV | 369350 | PSTVR |
| 246747 | IFTAD | 305648 | SCKIS | 345774 | SSKQR | 369358 | QHPSL |
| 246785 | NLPLD | 305651 | SKRSP | 345783 | AWATW | 369360 | VVFHL |
| 246789 | DYRMR | 305653 | AEVKS | 345785 | NPTHL | 369371 | ESVTR |
| 246792 | PCVLL | 305654 | DDQTS | 345789 | NRRRR | 369372 | TDPST |
| 246794 | LRRPH | 305661 | RLEDE | 345796 | HSRLG | 369374 | PPLLP |
| 246798 | HSRCG | 305675 | VHQTS | 345797 | CKKER | 369380 | KCFEN |
| 246801 | SAPEQ | 305682 | GTVSE | 345806 | SLPSS | 369386 | LKAGS |
| 246802 | REIQL | 305687 | GMLPP | 345808 | SIFGR | 369390 | SNSKH |
| 246841 | DYSYT | 305692 | LVSWC | 345809 | EIQKI | 369395 | LCEEN |
| 246868 | DEKFE | 305696 | GASRE | 345834 | ELNTK | 369399 | AEHRP |
| 246891 | VMLQW | 305699 | PATAR | 345841 | PLPWD | 369400 | SQSST |
| 246895 | QQYTF | 305702 | YLQED | 345848 | EGEDD | 369402 | STSGN |
| 246896 | YLYDN | 305711 | LLIDT | 345849 | RIGFF | 369405 | VLFIC |
| 246911 | TSESG | 305714 | LHSRK | 345853 | ELAPT | 369409 | LITCV |
| 246912 | KNQLY | 305721 | NKHGC | 345856 | FSLKF | 369411 | EGMMF |
| 246914 | DVGRM | 305725 | YLKLE | 345868 | AGGYP | 369418 | TLAQS |
| 246949 | EVMLK | 305731 | RCQES | 345869 | CGPVP | 369419 | RNSKH |
| 246957 | ALERH | 305764 | KESHS | 345873 | VETKF | 369421 | LVFKD |
| 247001 | RRPYY | 305766 | SRLEQ | 345874 | LVSLV | 369423 | ILNLL |
| 247003 | SQGLV | 305769 | ISSVS | 345886 | LSDID | 369424 | EGQGS |
| 247005 | ECGCR | 305777 | AQRHK | 345895 | EKKDA | 369431 | IKKPA |
| 247020 | HHAEL | 305785 | TRVSQ | 345900 | HSTGR | 369432 | ISSCC |
| 247026 | EKEDD | 305790 | TRYAF | 345917 | VKLVK | 369434 | HSAMT |
| 247029 | NVDHS | 305810 | DWYDS | 345920 | KLWFL | 369437 | MFSLH |
| 247087 | TVTSL | 305824 | NCTAK | 345924 | GKKLR | 369441 | FRLRK |
| 247138 | KVKGS | 305826 | SIAQE | 345964 | TYVNV | 369442 | RRSYK |
| 247153 | EEEEL | 305839 | GPTAE | 345972 | QSWSP | 369445 | QKSSC |
| 247170 | TKLNF | 305847 | PKIHP | 345980 | LLGSL | 369449 | SLTSP |
| 247178 | YTGHR | 305852 | GGGPV | 345984 | QVVPL | 369456 | GSLAA |
| 247182 | VDLGS | 305859 | TGCFC | 345985 | MESTV | 369458 | YSLGV |
| 247194 | GFLLK | 305873 | SDRLQ | 345988 | FLLRL | 369459 | RLRTR |
| 247207 | IEEVD | 305875 | VMKDL | 346001 | KEEGA | 369461 | RKSKL |
| 247219 | GFKKA | 305877 | ETDPS | 346012 | QVIQF | 369465 | IESVD |
| 247225 | FIGIS | 305892 | FTSCG | 346015 | CVLVA | 369473 | CHHGY |
| 247226 | LNSVG | 305896 | KLYKL | 346017 | RDLQA | 369477 | SPAGR |
| 247270 | NKQIG | 305899 | LRLNA | 346022 | QQADE | 369488 | KWRLQ |
| 247271 | VMLAV | 305906 | RDPCQ | 346027 | YEFMA | 369490 | RGPGK |
| 247306 | SAPHI | 305913 | LLCPT | 346032 | CGGDD | 369491 | GSGPG |
| 247461 | KPRRE | 305918 | SSDSE | 346037 | FGLFD | 369492 | SLGLG |
| 247470 | DLERS | 305919 | PETIN | 346039 | AGSIA | 369494 | NSVSI |
| 247523 | SNNSF | 305924 | RKSNP | 346045 | PRGPV | 369497 | TKKYI |
| 247550 | LKSHL | 305954 | AFKGM | 346046 | PRGPV | 369500 | LQEEN |
| 247584 | VPPNA | 305969 | MKNNL | 346048 | RRRLS | 369504 | GSGPG |
| 247618 | KKRSN | 305970 | HRPLH | 346050 | VQESV | 369508 | PASPA |
| 247655 | QLLKT | 305973 | DITRL | 346051 | AEHVK | 369512 | HCVRF |
| 247706 | PEDKK | 305974 | LKTKL | 346063 | IRQLN | 369513 | SKIYV |
| 247712 | DTRAD | 305975 | SEPTC | 346065 | EVMEI | 369514 | IPAAL |
| 247815 | DNQET | 305976 | YVLVN | 346067 | TTDWS | 369516 | DCGPS |
| 247829 | QIGNK | 305988 | HKTEA | 346080 | SESED | 369518 | KAKTS |
| 247843 | QAKDI | 305995 | ALSNM | 346085 | TPPCT | 369519 | LLPRH |
| 247866 | PDDED | 305999 | RRRWT | 346088 | EEDED | 369529 | MTMSV |
| 247879 | GPIFS | 306003 | DSILK | 346090 | SLYSL | 369530 | NSVSI |
| 247881 | LCFKK | 306010 | ELSKR | 346103 | LPHYF | 369531 | DLLSA |
| 247883 | RCWGP | 306015 | WRRQT | 346112 | ITIYS | 369538 | IYGED |
| 247930 | THTGE | 306070 | MKGID | 346114 | QQPPR | 369542 | SKRRK |
| 247933 | SPGNP | 306080 | DRSYH | 346120 | KAFGQ | 369544 | PTQTH |
| 247956 | WKRLQ | 306088 | GRVQD | 346123 | PLSLP | 369545 | LKIQN |
| 247970 | ILRTE | 306095 | YSSGT | 346127 | SPCWF | 369547 | KKISQ |
| 247977 | MDWWM | 306099 | FFPQQ | 346128 | QSEST | 369553 | LKSKE |
| 247986 | GSEPL | 306100 | APSSS | 346130 | RWTVS | 369554 | LRSKE |
| 247992 | HKPWC | 306105 | HAPTC | 346142 | LFYDV | 369556 | LRRKE |
| 248041 | GANSQ | 306106 | EKKHK | 346144 | LPPAP | 369558 | LRRKE |
| 248058 | FRGKQ | 306117 | EKIAN | 346146 | LGCCL | 369559 | HKSQA |
| 248069 | NRYDV | 306123 | GVSET | 346147 | RSCLL | 369560 | LRRKE |
| 248071 | MKRHM | 306124 | EDLMP | 346148 | KILAQ | 369562 | LRRKD |
| 248073 | TAGLS | 306129 | PNGFH | 346151 | SAASR | 369564 | LRRKD |
| 248076 | SSLLQ | 306138 | SSSSL | 346152 | SILIH | 369566 | LRRKD |
| 248089 | DIMEL | 306157 | FYQNQ | 346155 | VAYTG | 369568 | PHSRA |
| 248098 | SISFY | 306160 | LNLTL | 346160 | QENSS | 369571 | LRRKD |
| 248114 | DGSCD | 306163 | PVPAQ | 346167 | FWDPE | 369573 | GQSRA |
| 248121 | QVPAY | 306178 | ISIFQ | 346173 | EIGRI | 369574 | LRRKE |
| 248125 | ARIIT | 306185 | KELTA | 346191 | GQVGG | 369578 | DLGSS |
| 248139 | NCKIS | 306187 | CGKAF | 346193 | HVLQK | 369579 | PQPPE |
| 248142 | LCEYS | 306190 | TLPES | 346196 | ESPRH | 369580 | PEASV |
| 248150 | KCTIL | 306220 | ESLRR | 346203 | GISPS | 369581 | GYLRN |
| 248151 | EGGGV | 306223 | TPRGG | 346204 | KKKPP | 369589 | YRAQS |
| 248211 | RENAY | 306241 | RLSHF | 346206 | ADAPE | 369592 | FKGPL |
| 248228 | CFRDE | 306245 | TLLAL | 346228 | SCCLA | 369593 | RTHIP |
| 248244 | TQEAE | 306275 | RRSSV | 346229 | SAESE | 369597 | QSFQG |
| 248248 | LFTGL | 306276 | APNEC | 346236 | RYKVL | 369606 | QSFQG |
| 248272 | RVHSP | 306296 | KNPKK | 346240 | VWVEP | 369607 | SGTSL |
| 248306 | LKKQQ | 306299 | EEAEG | 346250 | DDWES | 369611 | ASSLR |
| 248342 | MASSQ | 306306 | ERMKH | 346251 | FVIKV | 369614 | GGVWR |
| 248378 | MVHVY | 306320 | PKTPS | 346253 | LFHYR | 369620 | HPRLF |
| 248384 | NPFLL | 306328 | EDRTQ | 346255 | AEYTD | 369622 | RWYIH |
| 248419 | LSRKD | 306330 | GEGNN | 346256 | GHRVS | 369625 | KYTIR |
| 248437 | DEGEE | 306335 | SLQGI | 346265 | LLPAP | 369626 | GPTQI |
| 248444 | EKGLF | 306340 | MEHQH | 346269 | RKATR | 369634 | LDDHQ |
| 248450 | QRPDR | 306344 | PTEFS | 346272 | KASSQ | 369636 | TDIWD |
| 248550 | WKIKS | 306356 | KHLPS | 346277 | DRVSF | 369638 | AKAIS |
| 248553 | ETAAK | 306361 | PLVTQ | 346283 | PRHAR | 369639 | CPGCS |
| 248564 | SCVIS | 306382 | EVAVP | 346291 | TETNA | 369643 | HERYV |
| 248566 | KMETS | 306386 | MPEGG | 346294 | QPRKK | 369645 | FKKAY |
| 248572 | GCVIS | 306390 | LGGQK | 346298 | ADPVA | 369648 | TSPVV |
| 248594 | PSEWT | 306396 | SMLLQ | 346300 | PDENE | 369649 | MEEVD |
| 248598 | KHFKP | 306397 | MVIVG | 346305 | DSVLF | 369658 | FPEKF |
| 248633 | KVTLA | 306407 | RPQED | 346306 | NVQDT | 369673 | GEKAQ |
| 248643 | LSRFA | 306410 | KARYT | 346310 | RDDQL | 369677 | ELEHA |
| 248668 | LESTV | 306425 | GNMPY | 346313 | SPPLS | 369681 | GSDKL |
| 248673 | ISVSE | 306446 | IQIHH | 346316 | YGFGG | 369682 | QRRDS |
| 248701 | RNGPC | 306449 | VGTHC | 346317 | VVSPR | 369687 | LVQAP |
| 248706 | TVTSV | 306459 | PIEDY | 346324 | TDEDD | 369689 | MLAQE |
| 248846 | YYQDA | 306461 | KTKQK | 346325 | ATPEA | 369690 | LSAAW |
| 248879 | SPVPP | 306469 | HRKCF | 346335 | LLLED | 369695 | TSGTS |
| 248901 | IASKQ | 306472 | ADQST | 346340 | AFLEN | 369696 | WRLQA |
| 248903 | LGVPI | 306473 | GTFLF | 346342 | PGEAK | 369699 | RLTPL |
| 248923 | EPAGY | 306482 | KGVDV | 346345 | HRGGH | 369703 | GEDEA |
| 248924 | HGALP | 306490 | PSRQC | 346348 | NTHLI | 369714 | CLYLH |
| 248933 | YEVSI | 306496 | MTCWC | 346363 | WFGMQ | 369716 | ICIDD |
| 248935 | LAMIR | 306497 | RESAI | 346371 | CPTPH | 369719 | RKAKT |
| 248958 | GHDEL | 306523 | LRPGC | 346378 | LLHMP | 369724 | HGTGK |
| 248975 | AGEGN | 306524 | KMEEE | 346388 | NLLTL | 369727 | KDQTH |
| 248983 | PGEAK | 306535 | LHIAT | 346401 | KRRPD | 369728 | VFTKK |
| 248984 | IFSES | 306548 | EDYRL | 346402 | LRVIS | 369729 | CQWKI |
| 248992 | SSCPK | 306549 | HPNYN | 346403 | SIGRD | 369730 | FSFFN |
| 248996 | YIGLC | 306561 | SLLAL | 346408 | FENVI | 369731 | CQRKI |
| 249007 | PGEAK | 306565 | LAVSD | 346412 | DDLDI | 369733 | LYVHI |
| 249014 | DEVKV | 306574 | FDSQP | 346415 | DHKDH | 369736 | VKLEA |
| 249016 | ESKGT | 306596 | DLLSS | 346417 | ARAQT | 369738 | RGRWD |
| 249041 | ARGPE | 306598 | ELVPQ | 346437 | DDETI | 369739 | EDFLQ |
| 249042 | KSEKA | 306606 | TVLTF | 346442 | GPPPV | 369742 | CGNIP |
| 249044 | GRHRQ | 306607 | MSKRS | 346450 | APGED | 369744 | LLLTK |
| 249062 | EPAGY | 306614 | QCGEM | 346453 | VLGLS | 369747 | CGKVF |
| 249064 | EEMEL | 306625 | PASKR | 346466 | LAHKA | 369750 | QSFIL |
| 249066 | PGQDQ | 306627 | KVRKE | 346467 | DSDLE | 369751 | HCLKL |
| 249071 | ACSLL | 306637 | SRSSG | 346483 | AQAPA | 369755 | ALLLL |
| 249075 | LAQAF | 306651 | SENKG | 346490 | DEAYD | 369757 | ESSQK |
| 249079 | GLATT | 306654 | LKLAL | 346500 | HQKAL | 369762 | RDLKT |
| 249116 | QNQGN | 306657 | IGFIF | 346508 | RLKPT | 369770 | RAHLS |
| 249122 | AHFNH | 306662 | APGQF | 346509 | APGED | 369774 | WRKTL |
| 249269 | CWLRD | 306670 | LPASS | 346510 | NSALT | 369775 | TEAVG |
| 249284 | LKGKC | 306678 | AEMEI | 346511 | PVCFL | 369777 | AAGRR |
| 249289 | AEDLR | 306682 | TVCVC | 346517 | AGQEM | 369778 | WKETL |
| 249299 | NSVAH | 306688 | KRKLI | 346530 | GEAQV | 369781 | TEAVG |
| 249330 | LLRRP | 306697 | SQLQS | 346534 | RESKC | 369782 | RAHLS |
| 249344 | LLQNH | 306705 | FSFFF | 346536 | LLGNP | 369785 | SKIGN |
| 249356 | DCSGQ | 306707 | YLFPL | 346537 | PRKQG | 369790 | RAHLS |
| 249363 | QETQI | 306726 | ISIDF | 346542 | STDYV | 369796 | SNLGS |
| 249364 | RHDEF | 306734 | TRYMT | 346543 | QADHW | 369798 | IGRKK |
| 249373 | ADSDF | 306752 | RERQK | 346550 | CGGED | 369802 | GAPLI |
| 249389 | QVGPN | 306754 | AFFHN | 346560 | LLLPH | 369810 | QAQKK |
| 249399 | PRLLP | 306760 | LSPPK | 346564 | KGWCV | 369816 | TDASH |
| 249440 | KLTHL | 306761 | EIQRV | 346566 | KSSRK | 369820 | ISETR |
| 249442 | KGRLS | 306772 | PQASQ | 346567 | AIRSY | 369823 | SVSPS |
| 249480 | RAKAN | 306776 | TLKHH | 346572 | RRRLS | 369833 | KLAGK |
| 249499 | CPKGD | 306780 | HLAQN | 346575 | VKVSY | 369834 | QVFYE |
| 249501 | NLTFS | 306788 | EKKNE | 346577 | IDSIF | 369835 | SRFWR |
| 249504 | GNPLF | 306791 | LPNDS | 346586 | RGHCG | 369837 | FLLYQ |
| 249598 | ECGCR | 306807 | SVAVH | 346587 | GKIRD | 369840 | NKKKK |
| 249601 | ARAPK | 306816 | DVSCV | 346598 | TTDWS | 369841 | ATCES |
| 249636 | IISLD | 306817 | PLTLF | 346599 | TAQNG | 369843 | VVSKM |
| 249647 | KLIDS | 306822 | DSMIS | 346600 | KASQD | 369849 | PNQFI |
| 249651 | RSKME | 306844 | EESSV | 346601 | AGPRK | 369853 | VEPCT |
| 249700 | EADRR | 306852 | PPALR | 346603 | AHPAP | 369855 | FLLHL |
| 249749 | IATEV | 306864 | AKPYI | 346608 | FKRCS | 369857 | RSPTF |
| 249750 | PQKNS | 306866 | SVYGL | 346610 | SEWEL | 369858 | LLLIP |
| 249760 | NADFH | 306881 | VSSAA | 346611 | RRNIF | 369860 | YLLYS |
| 249776 | QLLEM | 306884 | ERKNF | 346625 | CAVPR | 369862 | KPILF |
| 249798 | ALALV | 306887 | GKEIH | 346627 | SKNSE | 369863 | CQAQD |
| 249806 | VSSRH | 306888 | HVGRN | 346634 | QPTTE | 369864 | WSPQL |
| 249822 | SKLAG | 306894 | EGFAY | 346643 | AQKEN | 369866 | ATTGI |
| 249842 | FLTSF | 306900 | WLTVK | 346647 | AIHQR | 369870 | AQGLE |
| 249861 | KEETC | 306906 | GAMYT | 346652 | KILYE | 369871 | SDLTR |
| 249883 | VEILI | 306915 | AESKL | 346654 | KQKGL | 369873 | LPSES |
| 249887 | STFSI | 306919 | VASTV | 346659 | VVGWN | 369879 | FSEEY |
| 249910 | KLSLS | 306920 | WLDHV | 346666 | ASQSA | 369887 | PPRRF |
| 249923 | KKTSI | 306932 | AETVA | 346667 | PPGPK | 369893 | TSTYS |
| 250003 | IYQVL | 306939 | QEKHY | 346675 | HFVMC | 369895 | IPLKF |
| 250024 | TEDVH | 306940 | QSGDL | 346691 | FPPGF | 369897 | NGSFQ |
| 250056 | VYASP | 306965 | EANSP | 346693 | NKKAQ | 369899 | SLLMR |
| 250066 | YSMLQ | 306968 | FEMQA | 346695 | ASRFW | 369907 | LEVLA |
| 250076 | KEPSH | 306983 | LNEDQ | 346697 | GAGPR | 369915 | INPKV |
| 250092 | AYQAL | 306991 | MVLDV | 346716 | ALQVF | 369927 | YSDEY |
| 250101 | LFSED | 306997 | ESEDL | 346720 | RGSQC | 369932 | TCGKA |
| 250111 | RINKT | 306999 | YNFNS | 346733 | HLEES | 369943 | SSDEF |
| 250113 | PHRPN | 307004 | FMKRK | 346762 | FGYSR | 369962 | NTTVV |
| 250156 | RQNEG | 307006 | VQPYL | 346774 | VGPLI | 369964 | AAGGK |
| 250160 | SEIAN | 307014 | KKTMR | 346785 | KKLGK | 369965 | KNEAN |
| 250173 | VPPLI | 307023 | MPASA | 346789 | KVFMY | 369967 | TACVC |
| 250237 | GITLG | 307031 | CSPSS | 346791 | SCSSR | 369968 | EDDRD |
| 250244 | QLRTS | 307040 | ISSLC | 346793 | ELKDQ | 369970 | KFDLS |
| 250263 | PHFRK | 307041 | PSHLQ | 346796 | MSNFS | 369972 | SPPKV |
| 250340 | CEFPF | 307046 | KEFYA | 346797 | QPGAS | 369975 | AASPW |
| 250351 | SCQPF | 307052 | FLVKS | 346800 | SRLLV | 369976 | LSVDD |
| 250360 | IKIHR | 307060 | RPSVG | 346801 | THSHT | 369979 | RGQKD |
| 250366 | EIMEN | 307065 | SLSLM | 346802 | FGFNF | 369981 | VALPH |
| 250373 | EEPPA | 307071 | EAKKT | 346805 | RRIQQ | 369986 | VPLAL |
| 250377 | LHQKT | 307077 | FKFPG | 346808 | DRPDF | 369989 | EFGSA |
| 250378 | ILQAN | 307078 | GGKQV | 346809 | LCNFM | 369996 | IKGEK |
| 250405 | FFASK | 307080 | PPTPT | 346816 | ELPSQ | 370000 | PWKCV |
| 250416 | FLQLW | 307087 | YETDL | 346821 | TLEYS | 370007 | QAGES |
| 250448 | VLNTS | 307093 | IESSP | 346823 | SKFGS | 370013 | SLLAE |
| 250454 | LASHS | 307096 | VHFDG | 346825 | FLRPR | 370014 | QFSND |
| 250479 | GTETV | 307109 | KRYAT | 346827 | VESAF | 370021 | MKDIR |
| 250495 | GGLRQ | 307125 | GLGPA | 346829 | AASGI | 370023 | KKFYQ |
| 250498 | MNFVG | 307126 | AKDGL | 346837 | FTMGR | 370034 | FKETV |
| 250515 | KRRQC | 307129 | AKARK | 346839 | EDSRE | 370047 | FTSVD |
| 250524 | KHCSH | 307130 | CPLSS | 346843 | PDPLT | 370049 | DGTEV |
| 250535 | SLENN | 307132 | KCRKV | 346849 | RLYHL | 370055 | CCVIL |
| 250559 | SCQLL | 307134 | PDSWV | 346850 | HVFKS | 370056 | WRPKI |
| 250615 | RNSGC | 307142 | MPFYQ | 346860 | QTTRS | 370058 | AACVD |
| 250617 | TSILP | 307143 | YNWRS | 346874 | STMRY | 370063 | AGDAK |
| 250693 | GPGLL | 307155 | DSSRY | 346879 | YGRTW | 370070 | AGRPV |
| 250699 | LVQAL | 307156 | YNTCQ | 346886 | TEKDN | 370074 | RFSSP |
| 250776 | GVSRM | 307159 | RKIFS | 346890 | HKIVK | 370075 | GVDLL |
| 250784 | KQSSG | 307164 | CNEDL | 346892 | FCCWI | 370077 | TQIGL |
| 250805 | GVSRM | 307181 | PYGYY | 346901 | FLIFL | 370081 | YKSWS |
| 250823 | TPLSP | 307183 | DRRSR | 346902 | KNASC | 370083 | SHSLN |
| 250825 | TPLSP | 307184 | PGRGS | 346913 | KPARR | 370085 | CQGPL |
| 250831 | DSSRY | 307197 | TKRNC | 346918 | FIGVG | 370087 | TARAR |
| 250838 | KIDEF | 307199 | PPPSS | 346921 | LVMFI | 370088 | REEVD |
| 250863 | MLKSV | 307202 | LSSFR | 346927 | MEKDI | 370096 | FMTNQ |
| 250894 | SYTPE | 307206 | VIQNN | 346931 | LEILF | 370097 | PCLCL |
| 250896 | VGDHA | 307208 | QSTAS | 346936 | DLDLN | 370098 | VLAAL |
| 250916 | APAGL | 307214 | LSAGQ | 346937 | KEAPH | 370103 | LGLLA |
| 250930 | LYAVT | 307218 | RFFTI | 346942 | PNTQD | 370107 | AGRPV |
| 250937 | RGAPS | 307234 | SDDYM | 346955 | GRKSP | 370109 | STLDN |
| 250974 | PSQRA | 307235 | PLPSN | 346956 | SLFVD | 370113 | QDKVN |
| 251020 | EIVTS | 307241 | KTLNI | 346958 | EKIHF | 370115 | RFSSP |
| 251038 | PQTSE | 307252 | FQQGT | 346959 | RTVSK | 370119 | SHSLN |
| 251047 | AKKFF | 307258 | RVKSC | 346962 | LLSTS | 370125 | SFYIL |
| 251071 | LLYQD | 307259 | EETPM | 346964 | TCGAK | 370126 | FCCWI |
| 251074 | WVTEV | 307260 | GALKK | 346970 | LMGWE | 370128 | LDQCK |
| 251076 | ILDIL | 307264 | ADSEM | 346981 | IVKTT | 370129 | FSDEY |
| 251089 | EVTAP | 307265 | KERDP | 346984 | NSRGL | 370135 | AGRPV |
| 251102 | EEKAE | 307272 | EKRVR | 346986 | NSFMV | 370136 | PGAVS |
| 251108 | RLGIH | 307275 | AFRAP | 346987 | PPIGN | 370137 | NPLLE |
| 251119 | PGSRK | 307280 | HIMSI | 346990 | TCNRH | 370142 | RAHLS |
| 251127 | DLLDI | 307288 | RITFV | 346992 | TEKYR | 370150 | KQRRC |
| 251143 | LQTRT | 307292 | MESVA | 346997 | RGYLN | 370151 | ESDGT |
| 251152 | RRSAA | 307297 | EACQQ | 347003 | LFLIY | 370152 | FHLLG |
| 251166 | EDEWD | 307298 | FVGFH | 347005 | EAGEI | 370155 | YLRAS |
| 251170 | SLVSR | 307304 | NENDE | 347008 | ISYRQ | 370161 | QQMIA |
| 251181 | ERFLI | 307305 | LIEEC | 347009 | RQQNF | 370166 | NQSAK |
| 251203 | SSTSN | 307321 | NFNFT | 347011 | PGQSQ | 370191 | PGTFN |
| 251241 | RKETG | 307340 | KTKQK | 347017 | AIHSY | 370192 | SDRAC |
| 251250 | GKLTH | 307342 | FASNL | 347032 | TNIFA | 370194 | TNPNA |
| 251268 | TSMSL | 307356 | RTRSC | 347038 | PQRSR | 370195 | TFMYS |
| 251269 | GRKAI | 307369 | HSKLH | 347040 | TGTQR | 370198 | FLKFF |
| 251272 | FLNDM | 307397 | FRGKQ | 347041 | SLIEI | 370201 | KDHNS |
| 251275 | LLNPT | 307423 | YRRLQ | 347043 | HILHS | 370210 | QVYAG |
| 251287 | LSSNL | 307432 | SVAKK | 347045 | FSNIK | 370213 | SIMFN |
| 251289 | TRPAK | 307443 | RSSVL | 347046 | QAKRN | 370215 | GFPSQ |
| 251296 | ILELT | 307445 | LNELN | 347047 | TKGEV | 370216 | VLCPP |
| 251303 | DLLET | 307447 | AHSTL | 347049 | LMARE | 370222 | KLGLL |
| 251312 | TEQVT | 307449 | TVGRD | 347050 | KSKTK | 370223 | QTKGS |
| 251334 | SVSGI | 307461 | CTMKS | 347051 | SEITE | 370224 | LYDDS |
| 251337 | DCGLF | 307477 | ALSVP | 347053 | QVPRK | 370227 | VHAIA |
| 251343 | LSLNF | 307481 | DAARQ | 347055 | HIMSG | 370228 | TDSIE |
| 251363 | HTTAT | 307491 | RQKST | 347056 | LASVG | 370231 | KPSSD |
| 251372 | SQRSL | 307508 | SEPPM | 347057 | CGVGS | 370238 | SSRLQ |
| 251377 | DAAGR | 307513 | NEQQE | 347060 | LANSS | 370242 | PADDV |
| 251390 | SKAGE | 307515 | IQPQT | 347064 | AAKKD | 370243 | WGPGV |
| 251412 | GTQEQ | 307519 | SKTGI | 347066 | CKKAI | 370251 | LQGRL |
| 251413 | GTQEQ | 307525 | ASMLT | 347068 | AGGCI | 370253 | SPAFW |
| 251472 | APPGP | 307540 | RCQEA | 347081 | LNSTF | 370254 | FSDEY |
| 251473 | PAVAT | 307549 | CRQIN | 347087 | DESWD | 370255 | YFWPS |
| 251496 | NEDLS | 307552 | SRNPL | 347088 | AENNS | 370256 | DCSKF |
| 251507 | KFVYL | 307567 | DPGKV | 347089 | LPRGC | 370258 | SQTAY |
| 251527 | PQAMT | 307598 | KNIIL | 347095 | AENEF | 370259 | NKEAV |
| 251535 | NSVTI | 307599 | KVHCD | 347096 | VPSDL | 370263 | CSRKL |
| 251566 | IEKRE | 307604 | PSTLL | 347102 | YEVIY | 370264 | WWQGL |
| 251582 | MLGKF | 307607 | QKKKL | 347106 | DHYAT | 370265 | QKTLL |
| 251595 | TSKYR | 307617 | TAGAN | 347117 | GTEVA | 370266 | QHKDE |
| 251607 | YIKKT | 307634 | VPTII | 347119 | PVVCQ | 370267 | APAVI |
| 251630 | TVEFS | 307640 | PRDSR | 347121 | NHESI | 370270 | LWLTM |
| 251636 | KTENN | 307650 | AGAGQ | 347127 | HLGRH | 370271 | CVPFK |
| 251642 | DLSLD | 307666 | DRPKR | 347134 | KETLV | 370273 | RVKWC |
| 251643 | IEELM | 307674 | GIEFI | 347140 | TGEVA | 370274 | HRHTH |
| 251648 | KGCCN | 307684 | KLLDG | 347150 | IKIIN | 370277 | LTRPL |
| 251654 | ANIPL | 307694 | HPQMY | 347152 | QGTCV | 370281 | RTRLT |
| 251675 | MSLDS | 307697 | WRSKL | 347155 | SEKLC | 370284 | HSGER |
| 251691 | YDIIV | 307701 | VRASW | 347156 | CASWQ | 370288 | VTUSS |
| 251722 | VGSFR | 307705 | AAKKK | 347161 | CYRDF | 370290 | IRERK |
| 251749 | HGKFL | 307713 | AGSRQ | 347164 | CGGDD | 370293 | FIPAL |
| 251757 | VLKET | 307720 | GGAWL | 347168 | YGFGG | 370294 | HKKAL |
| 251775 | CFSKM | 307722 | KTQGH | 347169 | FEIEL | 370297 | PGVSA |
| 251776 | RVWLE | 307726 | GREVG | 347170 | GTSAS | 370299 | PERGK |
| 251808 | TLMEI | 307734 | FWKGQ | 347178 | SNLTR | 370301 | PMKRK |
| 251809 | RQYEL | 307741 | SRTIQ | 347184 | KVTTC | 370303 | FANHV |
| 251810 | LDADF | 307746 | GQRRE | 347188 | RKTDD | 370310 | GGSCL |
| 251822 | SSAVV | 307751 | RLNEK | 347190 | AMDSI | 370313 | TVSNC |
| 251847 | HLTRL | 307759 | ELAHS | 347193 | GLTAL | 370314 | TTQTT |
| 251849 | RLPVF | 307766 | CREHT | 347197 | KTQAV | 370316 | THGDL |
| 251864 | GGKPL | 307774 | KKDPR | 347198 | KSCTM | 370318 | SDLIS |
| 251871 | SPCLL | 307786 | KATNE | 347205 | TDTLC | 370331 | RSRWL |
| 251900 | EGKYS | 307798 | DSDLE | 347206 | VLFWK | 370334 | HWLGE |
| 251921 | LNYIC | 307801 | NPVFD | 347211 | DQCCC | 370336 | FHHVG |
| 251959 | EGDES | 307803 | YSRWI | 347213 | LPSTI | 370337 | FVNLD |
| 251968 | LSDLY | 307805 | GCECC | 347216 | LLSQT | 370343 | SSIQE |
| 251973 | SSSEA | 307821 | ELVGL | 347222 | LIISY | 370345 | ANGQL |
| 252011 | KVQDP | 307822 | NSSEI | 347232 | SYAFS | 370356 | HFGAL |
| 252015 | DFTEE | 307831 | GTARS | 347234 | RIHTG | 370357 | RRNFN |
| 252032 | FELKP | 307833 | LGSSK | 347243 | DISNI | 370368 | ADAAR |
| 252037 | TAGES | 307843 | CKKDS | 347247 | VHSGE | 370372 | GLHAP |
| 252071 | SPPFH | 307852 | VPVNR | 347250 | IAKTQ | 370373 | LLNLE |
| 252087 | KKEKK | 307853 | YGPLT | 347254 | AARPV | 370374 | QVEDS |
| 252102 | LSGKA | 307854 | YYREK | 347269 | LQELE | 370375 | KYVLS |
| 252108 | KEQLE | 307858 | KGDVG | 347276 | SSAEE | 370376 | STGKN |
| 252115 | FKIKL | 307859 | KDKVR | 347277 | PLPSQ | 370377 | SWIIQ |
| 252134 | SLNWS | 307860 | QLSLT | 347280 | SPRKP | 370378 | HFSGS |
| 252136 | TFPSS | 307863 | RRDFW | 347287 | LSASS | 370388 | ETHLW |
| 252137 | ASDFF | 307870 | PPAHL | 347294 | ELEYL | 370391 | SELDN |
| 252172 | QKIEE | 307875 | PSVEI | 347298 | WAICP | 370394 | STSPS |
| 252173 | KISAE | 307879 | SGGPN | 347299 | LHIRS | 370395 | AEEHS |
| 252206 | ENLLM | 307885 | QPTEQ | 347301 | GTLLN | 370398 | SSTCL |
| 252207 | VAELV | 307889 | VEKKK | 347308 | NLLLL | 370400 | LPLLF |
| 252211 | NILSQ | 307900 | FQYKN | 347310 | RCQAA | 370408 | PTVTQ |
| 252229 | STEGT | 307908 | IIHTR | 347313 | WKTVW | 370414 | KVKKE |
| 252242 | KSFKS | 307911 | RKPRL | 347314 | KKKDK | 370415 | LRVLG |
| 252244 | SGVTR | 307928 | KIINI | 347318 | SLHGH | 370418 | QETFT |
| 252245 | KSYTH | 307933 | NVELT | 347321 | SAGSS | 370419 | VHLMQ |
| 252250 | KSYKH | 307940 | FLDKL | 347322 | AFSEL | 370420 | ELPSV |
| 252252 | KSYKH | 307951 | MKSPP | 347324 | DKAEL | 370421 | PQEPR |
| 252268 | NQNSS | 307954 | NHPRR | 347329 | NKLGV | 370424 | NSSVY |
| 252288 | LVTKG | 307958 | FSIKE | 347339 | LGHEV | 370430 | ENWFE |
| 252308 | NSKEG | 307971 | APTSR | 347345 | WWTSN | 370439 | CCWVG |
| 252318 | QTNSQ | 307973 | RRELK | 347358 | EGEGV | 370443 | TCQGH |
| 252321 | RETDL | 308012 | ARRRC | 347371 | ALDDD | 370445 | YLYDN |
| 252322 | SEGPV | 308021 | NFPIY | 347372 | TGTQR | 370446 | GQEGP |
| 252325 | RRKQH | 308022 | EKMLR | 347379 | DRQKT | 370453 | SNYSS |
| 252329 | LLTSS | 308024 | LNEEN | 347389 | YVQFT | 370460 | KKERR |
| 252338 | LTREE | 308057 | YEPEA | 347402 | PSRGA | 370461 | REGRN |
| 252444 | EDDVA | 308080 | LHIAT | 347408 | FQVRT | 370467 | LPSYE |
| 252445 | EAANQ | 308082 | QRPSP | 347409 | RCQLW | 370468 | PGLDP |
| 252453 | AALPA | 308083 | FKTMH | 347416 | TLAKC | 370469 | KIPQK |
| 252455 | DHDEL | 308107 | VAACI | 347418 | VKRNV | 370473 | SMQSK |
| 252456 | YYNSA | 308117 | CRTSV | 347427 | CIMNW | 370479 | ELPSV |
| 252463 | RETLY | 308137 | LWLLS | 347428 | FSVKM | 370480 | QISRD |
| 252482 | PRAQR | 308149 | DGEES | 347431 | SLSKC | 370483 | STTRV |
| 252483 | RAMYV | 308159 | NKVLS | 347433 | WNIRR | 370486 | KGKRD |
| 252486 | PSDNH | 308161 | EKKIQ | 347438 | APKTN | 370488 | EKPKK |
| 252487 | GLTIG | 308164 | PECSP | 347440 | LVQLC | 370493 | RPRAL |
| 252489 | DKDQG | 308165 | SKTIK | 347441 | YFHRK | 370495 | AICVK |
| 252490 | LKGEE | 308176 | MDEES | 347443 | QGKPG | 370496 | LLATV |
| 252491 | LKIDS | 308179 | ILTLT | 347444 | LSKVN | 370499 | PLPAL |
| 252505 | FKANP | 308190 | PSWTT | 347451 | NMKMI | 370502 | GSEPT |
| 252506 | ITLPE | 308193 | QVPED | 347454 | HGGVL | 370503 | DQDSA |
| 252510 | DEEED | 308196 | RKRKQ | 347457 | SSLLD | 370508 | NVLHQ |
| 252512 | NDMMS | 308208 | LLDKV | 347458 | ACFMG | 370514 | RRIKE |
| 252519 | VQTSF | 308219 | LIVFL | 347463 | VVPPM | 370517 | QPWSV |
| 252520 | FASLF | 308220 | LGTQS | 347464 | KENNN | 370521 | HSLQH |
| 252530 | KKKKK | 308226 | VMRLG | 347472 | FSQDP | 370522 | LPMAP |
| 252542 | FTRRY | 308236 | ISQPN | 347474 | KILHC | 370526 | EDDPQ |
| 252543 | AAKKD | 308252 | KLTHL | 347481 | SMHRR | 370531 | KHSKI |
| 252575 | EGNFC | 308258 | APTPN | 347486 | TEEGD | 370532 | VLTLG |
| 252576 | FMWYT | 308268 | LSHFK | 347495 | SFDFQ | 370537 | VYFSL |
| 252590 | APSSG | 308270 | VGWSV | 347498 | AGQEH | 370541 | GGQIT |
| 252593 | SALLQ | 308275 | TYGIY | 347501 | VFSIL | 370545 | QPLPS |
| 252595 | GAFAL | 308279 | TKSSQ | 347504 | TSEKP | 370546 | GDGTP |
| 252597 | GDTFL | 308280 | YREGF | 347507 | GLNEE | 370547 | TACHH |
| 252599 | ARDEL | 308292 | QQEKQ | 347516 | PGSKS | 370557 | RLKIS |
| 252602 | KSLSH | 308312 | EEDLA | 347520 | HVHRF | 370564 | VPNNV |
| 252603 | KHSTL | 308315 | EEETR | 347533 | KKKKK | 370565 | QDDAA |
| 252622 | QQQDA | 308318 | LGPYY | 347538 | SLEVT | 370568 | QSSSA |
| 252631 | RFPVM | 308330 | IILTC | 347549 | LNHPY | 370571 | LSAIG |
| 252655 | EEELS | 308332 | NAEGE | 347555 | MHYTD | 370575 | TLELS |
| 252660 | VETVV | 308339 | PTQPC | 347577 | NLKTP | 370588 | TLLEK |
| 252669 | DHMYH | 308344 | EILRV | 347581 | TSPTQ | 370589 | ASQED |
| 252674 | EAVAT | 308351 | STFSK | 347582 | PILPQ | 370593 | DHYAT |
| 252675 | AAWFT | 308352 | SGIVQ | 347586 | SFLAT | 370606 | WAVIL |
| 252677 | SCTCR | 308354 | RLCCL | 347591 | LTETS | 370607 | IPFIL |
| 252699 | GESDL | 308361 | STDTL | 347592 | KKAKQ | 370612 | AHLAM |
| 252713 | TKENS | 308368 | RQVLQ | 347596 | GPFSF | 370613 | GRWRL |
| 252723 | RTGDR | 308369 | SQGSS | 347601 | FVAQH | 370614 | HIFNM |
| 252725 | DLKIK | 308405 | QQKTA | 347602 | AVECR | 370615 | NSDCR |
| 252729 | GHRAT | 308412 | VAFLA | 347609 | LLLSL | 370616 | DSSFL |
| 252744 | RERFG | 308413 | GRPGR | 347611 | TGEKP | 370624 | ALHSE |
| 252773 | AAPAP | 308430 | ESKSS | 347620 | TSTIF | 370629 | KFDLS |
| 252785 | RSVLS | 308450 | HQGIR | 347626 | GAVQF | 370639 | ILARK |
| 252797 | FQECG | 308461 | SCSIM | 347646 | LVFTY | 370640 | NSNYY |
| 252799 | PGLLG | 308466 | GTLTA | 347648 | KRGRK | 370658 | PVDIE |
| 252804 | AEEKP | 308479 | CENNA | 347649 | EPELA | 370659 | CKEMV |
| 252809 | DCHCI | 308496 | LELLS | 347650 | FSRLT | 370668 | AGGSK |
| 252816 | QAGKQ | 308533 | ADEPS | 347675 | LGMAS | 370671 | NKAET |
| 252818 | QVPAY | 308534 | HQNRY | 347687 | LPVDH | 370673 | MKENS |
| 252825 | ETPEP | 308535 | TSSMV | 347689 | DNQRT | 370678 | NRQEG |
| 252835 | SSNII | 308540 | VEVKF | 347695 | DEDQL | 370679 | KEILL |
| 252840 | HNRQM | 308541 | DQFGE | 347696 | NLVSL | 370697 | QESVF |
| 252857 | NDVDE | 308546 | RSPSH | 347698 | FIKQK | 370702 | ACLIC |
| 252889 | PTMPS | 308548 | EELPS | 347703 | KSWAS | 370703 | LDTNI |
| 252891 | FEIEL | 308549 | ERPDD | 347710 | PGDES | 370710 | CQKRQ |
| 252898 | FESES | 308575 | SNGMS | 347712 | ALLLL | 370713 | FLLTL |
| 252900 | QVRWT | 308576 | FCVVT | 347714 | PSVCK | 370718 | TKVAP |
| 252909 | CLVAR | 308578 | MWESL | 347716 | YITLT | 370719 | LFDEP |
| 252934 | DTPKP | 308591 | GPQGP | 347717 | LPGIF | 370727 | SFPFL |
| 252936 | AVTAQ | 308595 | IRKLY | 347721 | STEEP | 370729 | GGVAS |
| 252939 | PPPAQ | 308606 | IQSEL | 347722 | STCAH | 370735 | QSVSN |
| 252945 | VIPRS | 308610 | SCGGL | 347730 | DLCGK | 370736 | PDSGF |
| 252951 | TEKYR | 308620 | RRLFD | 347737 | FCDNN | 370740 | KRASS |
| 252971 | QPAPQ | 308621 | GLLTP | 347741 | LFPGN | 370744 | HMPPH |
| 252979 | GEASS | 308622 | SFWRS | 347744 | RAMLQ | 370748 | KIYRM |
| 252985 | PGLSS | 308683 | SLKFS | 347745 | LDSIF | 370750 | GLPID |
| 252992 | NCVTQ | 308685 | GPPLS | 347748 | KAINA | 370752 | ASMGR |
| 252996 | KAFLK | 308689 | QPQSV | 347754 | IWQYA | 370755 | ADGGR |
| 252997 | ALALA | 308695 | EVVKE | 347755 | LTEHQ | 370761 | TNKTN |
| 252998 | PWEET | 308699 | GPRFH | 347767 | VLILM | 370763 | GGAHL |
| 252999 | GCPAA | 308708 | KVESV | 347774 | PRQHP | 370765 | LAWLF |
| 253001 | GTGRV | 308714 | PARRP | 347778 | SNEDV | 370766 | GRPGK |
| 253003 | DMSLD | 308716 | ACGCS | 347780 | KLTQN | 370770 | GHGVS |
| 253004 | KVTAK | 308718 | FPPGF | 347792 | PLAKT | 370775 | FSCFS |
| 253008 | PAMVL | 308720 | IAANS | 347794 | MWVKG | 370777 | SILVY |
| 253023 | ERCLK | 308727 | EMEKV | 347799 | EWKHQ | 370779 | IKVSP |
| 253024 | PGDGP | 308733 | GEGVK | 347802 | SMEEE | 370781 | VWNCY |
| 253031 | SLAPS | 308741 | KVQPQ | 347810 | NRKKH | 370783 | QRSQN |
| 253039 | TVDDD | 308750 | NLRHY | 347817 | VLGKL | 370793 | VGSLS |
| 253047 | RPPPE | 308753 | EGDHQ | 347819 | VVSCP | 370794 | GEALP |
| 253048 | SPFCQ | 308759 | EQTHN | 347820 | EPMVS | 370800 | MLTGG |
| 253050 | PRKGD | 308761 | IYRKV | 347823 | RPAKV | 370801 | MLTCR |
| 253054 | LSFFR | 308764 | SNLGS | 347834 | MMPTW | 370803 | NVATI |
| 253055 | AHGSH | 308770 | FRTEI | 347836 | QDMEL | 370805 | YDEGP |
| 253063 | TRYMT | 308772 | VDTSL | 347838 | EGFAE | 370806 | YDEGP |
| 253079 | TFVSY | 308774 | EKDKH | 347845 | YSQEK | 370808 | LKKVI |
| 253083 | QLVNY | 308782 | SITLI | 347847 | WDSCL | 370812 | WLNPR |
| 253099 | TPYHC | 308820 | PQGLE | 347858 | KIFMS | 370822 | GNPRA |
| 253108 | KPSTN | 308821 | RITAG | 347864 | TFTFK | 370826 | LQEAK |
| 253109 | RPLKL | 308828 | HHANS | 347868 | LISIR | 370839 | CGNTH |
| 253110 | GGPRE | 308833 | PGSSR | 347872 | PEKEN | 370842 | KLSET |
| 253115 | HEQIH | 308845 | HVQKK | 347877 | FDNVI | 370843 | IFNFL |
| 253144 | RIHNS | 308857 | KDPSS | 347883 | LGSVC | 370844 | SPSDV |
| 253159 | LTHVF | 308870 | RNAYV | 347884 | CLLHH | 370849 | LEGEP |
| 253188 | PSKPQ | 308875 | SFPSF | 347890 | PSLLD | 370854 | GMELN |
| 253193 | ALLVC | 308887 | QYICK | 347896 | RKLAP | 370855 | GTHCR |
| 253233 | SKKVH | 308893 | TLEKS | 347900 | ILEIE | 370856 | GTHYR |
| 253237 | RTISV | 308895 | QNVNQ | 347906 | FPIWR | 370857 | KFDLN |
| 253247 | VLELF | 308897 | EAAKK | 347910 | EKPYE | 370859 | GMEWN |
| 253255 | VYLVV | 308901 | KPQLA | 347915 | IGWNK | 370861 | GMEWN |
| 253262 | RHCSQ | 308903 | YVTMN | 347919 | AGAHH | 370864 | LTPPA |
| 253270 | LDLKS | 308908 | IFTIS | 347924 | YHRGV | 370867 | IDVAE |
| 253303 | YSYAG | 308916 | TEYCR | 347929 | SGKKE | 370873 | QPSAS |
| 253320 | PLGID | 308921 | FSEPL | 347930 | DDDEE | 370875 | NLSPL |
| 253323 | TFTPL | 308925 | RTDCL | 347931 | LEHQY | 370880 | PPYSP |
| 253325 | TFTPL | 308926 | PGSYS | 347933 | VLFEK | 370881 | TLNFI |
| 253329 | KSKYR | 308927 | AGQAN | 347944 | PGSKD | 370883 | TCIHY |
| 253332 | ELTES | 308928 | MSLYK | 347948 | RSPNH | 370884 | LSGGL |
| 253335 | ILPGI | 308937 | LRKRE | 347959 | SDKHR | 370889 | KCFEG |
| 253339 | DLVYV | 308938 | VMRNN | 347965 | VVARC | 370890 | ATCLS |
| 253354 | SPVSQ | 308957 | KNNLG | 347977 | GEKIA | 370898 | DDQLC |
| 253362 | LQTLI | 308970 | SGNRN | 347978 | RKRKT | 370903 | QSFNI |
| 253363 | VPSRR | 308976 | TLHIY | 347984 | GQYDG | 370904 | GVNDP |
| 253375 | PGKRN | 308978 | EITLV | 347985 | LRLYT | 370920 | SESSL |
| 253381 | VHHSS | 309003 | PEPAG | 347997 | KLRQS | 370923 | PPHGV |
| 253382 | CLTIQ | 309007 | QDYIS | 347999 | FKDLN | 370936 | LEVSN |
| 253392 | EVPSP | 309012 | KKLLP | 348010 | PPPPT | 370938 | QTHRY |
| 253401 | TPFKK | 309020 | TLYDL | 348019 | HGHRH | 370943 | DCSVS |
| 253407 | IIYSD | 309031 | PMDHS | 348020 | PCEPP | 370949 | THDVN |
| 253408 | HKDVM | 309036 | TFKNW | 348024 | SFVDF | 370950 | VPGEN |
| 253410 | DAGEK | 309048 | VQGLL | 348027 | FRISP | 370952 | SAEEQ |
| 253413 | RKFLD | 309052 | EDFRN | 348031 | ESEQK | 370962 | PFQGF |
| 253435 | FIHFS | 309087 | KDTLV | 348033 | RKFLY | 370964 | KLHYK |
| 253452 | NEWKK | 309089 | KLRLG | 348043 | RAPLP | 370966 | QVVQF |
| 253457 | VLHLC | 309092 | PRIIM | 348046 | EEGWR | 370968 | SSSKK |
| 253458 | KGYPR | 309095 | MKFFI | 348062 | FKSVS | 370979 | WKLWR |
| 253461 | CPSAS | 309096 | SPRRC | 348064 | LFVAL | 370990 | EALGL |
| 253462 | QSQDF | 309103 | SAPSQ | 348067 | KTEKE | 370997 | QMQPI |
| 253470 | TTSRG | 309110 | DPASE | 348068 | NPTQK | 370999 | LYQIS |
| 253475 | RPAWP | 309116 | EEPSP | 348069 | TVTSS | 371024 | GKDEL |
| 253481 | AKQQA | 309117 | RFAPY | 348074 | LRSSV | 371029 | GDVSK |
| 253490 | IFNIF | 309124 | NLRAQ | 348079 | QGFRL | 371031 | TAPLF |
| 253496 | EHTVS | 309126 | TPPCT | 348081 | IQIFV | 371035 | RVSCK |
| 253513 | LLKEG | 309132 | HEEVD | 348082 | PRSAT | 371037 | GRSDH |
| 253571 | RESVV | 309141 | KETVV | 348086 | DKFRC | 371040 | LARFC |
| 253577 | GNCSC | 309142 | DLKWK | 348089 | KPEYC | 371051 | WNVEC |
| 253669 | GRDLS | 309148 | TIQAK | 348094 | SHPRV | 371054 | VTKIY |
| 253673 | VKRKY | 309161 | TGEEP | 348097 | GSPGS | 371058 | FVEGF |
| 253680 | APGYC | 309163 | APLCS | 348099 | AFSHV | 371061 | HHRCN |
| 253686 | ADAQD | 309166 | RYSAF | 348104 | RNKQT | 371067 | DNMAG |
| 253692 | SPLDI | 309175 | EEEAA | 348107 | GKSKS | 371068 | VQRLT |
| 253693 | ITQLQ | 309181 | PPGKH | 348108 | PYGRY | 371069 | WMRTN |
| 253697 | QASKK | 309186 | FVESF | 348109 | LVLQV | 371073 | KLLWC |
| 253699 | KGGTD | 309189 | KPSRV | 348110 | ATKKN | 371082 | DNLAT |
| 253704 | TPDCR | 309198 | GFELS | 348113 | KKTAP | 371084 | RPDEW |
| 253719 | AQFPG | 309230 | ILQHR | 348115 | HWAWG | 371085 | APLIR |
| 253720 | PIFPL | 309233 | TSASL | 348128 | AKGLF | 371090 | WNKSD |
| 253727 | WDVHE | 309249 | LTSTS | 348129 | GSKDD | 371092 | AKANF |
| 253754 | KVELV | 309252 | GKLHK | 348132 | ANFDC | 371096 | EINTS |
| 253778 | SVTVE | 309259 | VSAQA | 348135 | VICVS | 371099 | MGQRP |
| 253788 | QKLRF | 309262 | GLLGH | 348138 | AFGGQ | 371101 | GRSFH |
| 253789 | TLPDP | 309270 | FRPFS | 348145 | EDAMG | 371102 | LGHLL |
| 253792 | EHMSM | 309276 | PFSRE | 348150 | AEAAP | 371103 | ASSSS |
| 253794 | GVKFF | 309285 | AFLPS | 348153 | SGSIP | 371116 | QRCSQ |
| 253796 | SEAQA | 309297 | NVHPC | 348161 | SIMIC | 371120 | KQSQC |
| 253799 | SYHGF | 309302 | IQKIS | 348168 | ITSSK | 371126 | QRCSQ |
| 253801 | HKKSL | 309314 | PMGLL | 348170 | TIAEN | 371135 | SECDI |
| 253803 | SDCIM | 309324 | KKMEM | 348173 | SPAHR | 371136 | QSIRC |
| 253811 | VGRAS | 309330 | NFDNT | 348175 | SHSMK | 371152 | PCIGW |
| 253812 | SNLLQ | 309332 | PIQVI | 348183 | EDSDS | 371153 | KQSQC |
| 253814 | VLFIY | 309334 | LHRYR | 348186 | RARLS | 371155 | KRGKR |
| 253815 | QKYAM | 309336 | SKEAL | 348190 | SLRPA | 371161 | LLKLE |
| 253856 | GTAEE | 309358 | LCLFD | 348191 | MTAVK | 371169 | SKTLK |
| 253861 | KITTV | 309376 | NYHTL | 348193 | IICWV | 371170 | KQSQC |
| 253925 | RTYSC | 309378 | CCSHF | 348197 | KVGAR | 371173 | KQSQC |
| 253928 | DGESQ | 309392 | RSSCR | 348200 | QVIQF | 371175 | TWGSL |
| 253934 | VESPC | 309402 | KEKSS | 348205 | ILFAM | 371176 | ESTTV |
| 253968 | RSRED | 309415 | TPLSR | 348206 | AFAEK | 371185 | SLISA |
| 254029 | RKNVS | 309417 | QLGQK | 348216 | VTDRK | 371186 | SCSSP |
| 254035 | QFGKK | 309429 | GHTAS | 348218 | GPTRV | 371190 | DEAAS |
| 254037 | KVVNF | 309430 | PEKTD | 348222 | PDKCV | 371194 | GGPAP |
| 254043 | HKREI | 309431 | EEEVA | 348226 | DLEEL | 371196 | GGWPL |
| 254051 | DAERM | 309432 | SSRSH | 348234 | EECDK | 371197 | LGYWE |
| 254072 | QPSCC | 309433 | EETES | 348237 | GKAKK | 371200 | PDHQN |
| 254090 | IIAYF | 309439 | EQGKE | 348240 | CGQLE | 371201 | PRRWL |
| 254101 | LYKPI | 309457 | LEMKK | 348249 | GSDGL | 371202 | HTHTH |
| 254108 | RERPY | 309462 | NCKNY | 348253 | ENTDD | 371205 | PPRAP |
| 254109 | EPGPS | 309463 | EDSPN | 348258 | YGFGG | 371206 | LKKQN |
| 254122 | GEMKE | 309474 | EYIDD | 348267 | SCAQE | 371211 | QQVVT |
| 254165 | EATSQ | 309477 | RKMAK | 348273 | PMARR | 371212 | VYQTY |
| 254166 | KKIHT | 309482 | SLKES | 348278 | QLSVS | 371215 | STAGT |
| 254181 | LYRPA | 309487 | QFSHL | 348283 | GFGQE | 371219 | ECKIF |
| 254182 | RHHEC | 309493 | RMSSI | 348286 | HFVMC | 371220 | ERNAR |
| 254190 | SVRTA | 309501 | YAPDN | 348298 | ESKNE | 371221 | ARMTC |
| 254193 | VTNGS | 309503 | EGGEN | 348299 | KYKLL | 371224 | FVFKI |
| 254227 | MLLLR | 309504 | TMPPL | 348300 | LDRIL | 371230 | ASVTP |
| 254231 | PEAQN | 309509 | AMRTF | 348302 | FSYFP | 371231 | AWSRA |
| 254235 | GLGLN | 309515 | LAPPG | 348305 | VGPLI | 371232 | ASVFT |
| 254250 | VEVPA | 309520 | KVNAQ | 348308 | VRVRG | 371234 | REYCR |
| 254260 | DSSWY | 309521 | PASLP | 348312 | YELEG | 371236 | WVKEE |
| 254262 | FTQPR | 309524 | GSCGF | 348314 | TEEKK | 371242 | AEINV |
| 254271 | CIYFV | 309538 | QPRKC | 348316 | LDLFH | 371243 | KSSKY |
| 254279 | ILRIM | 309539 | ITSLG | 348336 | APQRL | 371255 | PLHQE |
| 254286 | FSTEK | 309542 | LAPVA | 348337 | SLLYR | 371272 | PELDF |
| 254299 | TLIRS | 309548 | YYKGG | 348349 | NEFMV | 371278 | MMDTV |
| 254301 | SYTMI | 309560 | AEIAK | 348379 | SINIR | 371281 | KSTPV |
| 254302 | ATSSF | 309561 | KARTK | 348380 | VSKRY | 371284 | LDRWG |
| 254320 | ILVSW | 309565 | FNNLK | 348381 | EKVEA | 371288 | TPSCK |
| 254321 | GKAFS | 309570 | MKRHI | 348384 | YATCK | 371290 | PQPPE |
| 254322 | QVLPI | 309572 | KTILD | 348394 | CLSEP | 371291 | GGSQL |
| 254323 | RERFG | 309576 | EVRSW | 348395 | RRRLV | 371294 | SSLKG |
| 254325 | ALPSS | 309591 | EFSEF | 348401 | QAASR | 371307 | LWSQR |
| 254336 | PWREL | 309595 | SKRSK | 348411 | EAHFV | 371308 | MDTEL |
| 254337 | TWIVL | 309597 | PEAGP | 348415 | HLQQW | 371310 | IKIHN |
| 254351 | EEFYA | 309600 | WGLNH | 348425 | WGFQP | 371318 | TLQRS |
| 254354 | GQIHR | 309606 | VEMGT | 348429 | EHIQD | 371319 | EQRYF |
| 254436 | GSTDY | 309610 | LSSTS | 348431 | CGPIR | 371321 | KSSKK |
| 254442 | HRFMV | 309619 | ILFIQ | 348432 | KQMAP | 371322 | NVKKN |
| 254454 | RSWRP | 309620 | KILNN | 348442 | IHNLQ | 371326 | CKHKL |
| 254457 | EAAVW | 309622 | SSSPE | 348449 | KRVLF | 371328 | DIMIA |
| 254466 | EESWV | 309629 | EGKPL | 348455 | RSSDK | 371333 | KQQLI |
| 254480 | ASAAP | 309640 | PIPED | 348459 | RKQFE | 371338 | PETSV |
| 254488 | KETHF | 309644 | SQDTS | 348460 | ERKED | 371341 | AHHKR |
| 254508 | AYASH | 309649 | EGTRS | 348461 | KCLLL | 371345 | AEPVT |
| 254521 | SGSCL | 309653 | IYTDT | 348463 | SSFLI | 371347 | RPLDI |
| 254528 | FLSHL | 309661 | TISEI | 348467 | LSYGA | 371348 | VDSWN |
| 254579 | GDAEG | 309671 | WGHCP | 348472 | APKIG | 371350 | TGDTT |
| 254584 | WLEEQ | 309673 | RKSPL | 348476 | EIDMA | 371352 | ADKDE |
| 254605 | LYKRR | 309681 | YRFKR | 348477 | DVGLG | 371356 | TGDTT |
| 254616 | SPSGS | 309689 | RLLGS | 348489 | ARADR | 371364 | PIPEL |
| 254624 | DLCVY | 309690 | VKFML | 348496 | YPKRD | 371369 | HTPRA |
| 254630 | TSEGK | 309697 | IQLPG | 348501 | NRETL | 371374 | RRSLT |
| 254636 | QVQPE | 309710 | LFIQI | 348510 | KWIHL | 371376 | NDVKS |
| 254654 | VRIGH | 309714 | LRKKP | 348512 | TTPKF | 371377 | KRISS |
| 254657 | IEEQT | 309741 | IVKEG | 348514 | EGRQL | 371379 | KIHSP |
| 254661 | TEGIV | 309749 | QEILE | 348518 | ECGKA | 371383 | FSCPW |
| 254663 | LEDQA | 309751 | GLEED | 348519 | LSVEN | 371388 | TPDLQ |
| 254667 | YANFK | 309757 | NKKSG | 348523 | LVEDN | 371390 | EELWT |
| 254691 | RGGKH | 309762 | NTVLW | 348527 | IFQIE | 371393 | QPKAD |
| 254695 | SGAGH | 309766 | EPMNL | 348541 | GHGKF | 371394 | PLPHS |
| 254712 | AQPQI | 309767 | LISTF | 348545 | PILPQ | 371397 | EPHSG |
| 254719 | RSALM | 309771 | QRQGR | 348547 | WLTEG | 371398 | VYTEG |
| 254722 | DPRGP | 309772 | NKRTM | 348549 | PTTSV | 371400 | ALSVS |
| 254730 | MVQSP | 309782 | PGNAS | 348551 | LSDSE | 371401 | KLGSH |
| 254759 | EKLKK | 309786 | AFGGQ | 348552 | KRFLQ | 371404 | RWLPG |
| 254765 | RYCDK | 309790 | NSDSA | 348554 | SDHST | 371405 | QDISP |
| 254770 | SSKRD | 309794 | FGFRR | 348557 | TGVSH | 371409 | WRKSL |
| 254781 | SQKHT | 309812 | GGKNL | 348562 | YQVLE | 371412 | HRAWP |
| 254799 | CPKGK | 309818 | SFSQS | 348564 | VLAQS | 371419 | HYSQG |
| 254800 | LLLQA | 309823 | GEELS | 348565 | KMKNM | 371420 | FKGVS |
| 254801 | ACYPD | 309826 | VTLEQ | 348577 | TLYKV | 371425 | VLQTL |
| 254803 | SIKLK | 309830 | VKELK | 348578 | LAPRQ | 371429 | FPMIT |
| 254806 | DKKTQ | 309831 | CLMEN | 348580 | PPKPM | 371434 | TYTAV |
| 254810 | RGERA | 309836 | KTGKQ | 348589 | THRLL | 371437 | IDQRF |
| 254816 | VLKRR | 309845 | KCVLS | 348591 | NLLTL | 371439 | KKKSK |
| 254835 | SRKTI | 309871 | SPMLK | 348593 | QTLSK | 371449 | DAMFT |
| 254846 | ASTSR | 309878 | GSVKT | 348596 | RKRMK | 371451 | KRKNR |
| 254853 | DPCGP | 309893 | RRAEM | 348610 | AIAAL | 371452 | EFKAE |
| 254854 | PGQFS | 309913 | WSDNS | 348611 | FDTAL | 371457 | VMPPP |
| 254867 | KRFHF | 309934 | RRPSQ | 348619 | RQFIA | 371462 | CKIEF |
| 254868 | SQESH | 309939 | SCSRC | 348632 | CQNCH | 371465 | MLFAL |
| 254878 | TTASL | 309945 | ASVLI | 348634 | MHDEE | 371466 | VPKQS |
| 254898 | RLRYR | 309953 | GAYTF | 348635 | TQQAF | 371471 | LKLDW |
| 254900 | GKPVF | 309968 | KETEC | 348639 | SKTGM | 371475 | DYVSH |
| 254908 | AAASV | 309982 | SPRGR | 348645 | QDCCA | 371476 | LPSRE |
| 254928 | VKLLK | 309985 | PDECV | 348646 | DAVID | 371477 | EKQDR |
| 254940 | EETLT | 310003 | TLVSF | 348657 | ELLNS | 371483 | ECTAL |
| 254942 | RLGMN | 310008 | SGGQP | 348659 | PGGKL | 371490 | RLSFM |
| 254950 | FGQES | 310015 | YHQFK | 348668 | LWLFV | 371493 | FGSPK |
| 254958 | MEYIV | 310036 | ADTEL | 348670 | HVHSP | 371502 | FVEGL |
| 254976 | MLGSG | 310040 | KLVNL | 348673 | LDSVM | 371505 | NKIYL |
| 254998 | QDWAS | 310042 | VQVRF | 348685 | YTGPR | 371508 | LSPPF |
| 255006 | DLTTS | 310048 | PTSTI | 348692 | CQYLY | 371510 | PDHSP |
| 255008 | RTTTF | 310057 | QIENN | 348695 | GCWNK | 371512 | ICLWS |
| 255030 | PQLWP | 310071 | YRSIR | 348697 | TEASS | 371514 | PRQEN |
| 255039 | CYAEN | 310073 | LSQTP | 348703 | GEKHS | 371517 | SNIQK |
| 255040 | PLVWV | 310075 | IGALQ | 348706 | YTSSK | 371522 | SSGAH |
| 255078 | KERGT | 310094 | LPPWR | 348710 | ATTLG | 371529 | GLLRL |
| 255082 | PSPAS | 310102 | RVVCL | 348714 | GFGML | 371532 | DDDLA |
| 255084 | SCTLL | 310110 | PPYSP | 348722 | TTWLR | 371536 | HKPLP |
| 255087 | GLKME | 310111 | TEIKI | 348729 | DLLTS | 371537 | MYLFI |
| 255108 | DEGSG | 310117 | TPQKK | 348731 | SCPVG | 371542 | TPFRK |
| 255132 | LANQK | 310120 | QRFMW | 348738 | LGIFI | 371548 | VDLSL |
| 255152 | AVMHY | 310126 | NMKMI | 348749 | PHQMT | 371553 | TPALK |
| 255174 | EMMYT | 310129 | PNYDL | 348751 | TNKSA | 371554 | ECKAA |
| 255175 | SRDFS | 310135 | EPEEQ | 348752 | SLQVL | 371558 | TVTLR |
| 255183 | YISVP | 310146 | RRLGG | 348753 | HKAAA | 371559 | YHILL |
| 255189 | GKDKT | 310149 | AVPRP | 348757 | RSRRS | 371568 | FPFCP |
| 255192 | SKPDF | 310153 | KHLLS | 348762 | LAGHP | 371572 | LFSSQ |
| 255194 | VLSQG | 310170 | TLLAL | 348763 | GFTMV | 371577 | DLGDV |
| 255198 | TKVLL | 310172 | KYVAV | 348769 | DFHDT | 371581 | THGLT |
| 255224 | VLCDG | 310180 | KSVFG | 348772 | LDSEE | 371582 | QEAFV |
| 255226 | AYDVS | 310182 | IYVQQ | 348775 | LKSKL | 371594 | HPPQG |
| 255262 | HFNKT | 310189 | TSKDR | 348784 | RQNMG | 371595 | CQCKI |
| 255263 | GPLLP | 310193 | LDTRF | 348786 | SCLLL | 371597 | RQALC |
| 255266 | SCCIQ | 310200 | WMGGQ | 348787 | MEAVP | 371600 | TLQPG |
| 255289 | STTPR | 310205 | NTSEA | 348793 | SAHLS | 371602 | AHDIR |
| 255304 | LFENY | 310208 | QETSL | 348799 | SENLT | 371603 | CQCKI |
| 255305 | LLCVK | 310216 | LICFL | 348801 | EEDDE | 371604 | CQCKI |
| 255380 | PEQAL | 310219 | IEEVD | 348807 | HVSGA | 371606 | CQCKI |
| 255381 | VISEE | 310226 | GGGCC | 348813 | PVHSS | 371607 | LMDSE |
| 255389 | SHKRS | 310241 | SSTYF | 348815 | PLSPS | 371611 | PSAGR |
| 255390 | YRKKS | 310244 | QGDCS | 348816 | VKVFV | 371614 | RREQP |
| 255403 | NPRPL | 310255 | GAGPT | 348819 | VPFHG | 371634 | SQRSK |
| 255409 | ALAAT | 310260 | HINSN | 348821 | GKKAK | 371635 | RPAVT |
| 255416 | ASAAH | 310263 | ATSCL | 348828 | LETNV | 371639 | SILIC |
| 255465 | VLLLQ | 310274 | PLKLQ | 348831 | PDSDP | 371640 | GNPAP |
| 255468 | QTLLS | 310275 | CDAFL | 348838 | TSQEP | 371643 | ILTGK |
| 255476 | PGTSM | 310301 | NETME | 348840 | GKFSK | 371644 | DPWVH |
| 255495 | NHILD | 310303 | LKNKS | 348842 | VMGWP | 371646 | WHFVM |
| 255499 | REIKS | 310305 | KDIRL | 348849 | PPKPM | 371650 | PSIWK |
| 255513 | LQKPH | 310309 | QARLF | 348859 | AHQLL | 371652 | KHRPM |
| 255557 | GRPLT | 310332 | KPLDG | 348864 | GEEGK | 371659 | SSKPV |
| 255608 | VIFYT | 310334 | RHQKS | 348869 | AGRPV | 371660 | LTPDG |
| 255612 | LPLGR | 310335 | YISIL | 348870 | ALATN | 371676 | GYIMR |
| 255613 | GGEEL | 310337 | EAKGK | 348873 | EDWRE | 371678 | FSTDV |
| 255622 | TCCIL | 310338 | SSPVK | 348875 | LANCF | 371682 | LTASL |
| 255631 | DSMDR | 310355 | VKLQC | 348877 | EARVQ | 371683 | AFASQ |
| 255641 | LQRHK | 310356 | HRNIP | 348886 | RVKMS | 371687 | REGVL |
| 255674 | LLNSS | 310357 | QVNTC | 348888 | GPQEA | 371693 | RLGSL |
| 255681 | YFPVA | 310360 | EEEDN | 348892 | KPHLS | 371696 | VACGD |
| 255684 | YCVHS | 310375 | VQMAQ | 348897 | LGVSP | 371706 | SHLLE |
| 255688 | KKATA | 310382 | QNVFI | 348901 | ACRLL | 371707 | CNFIS |
| 255695 | KRERQ | 310393 | NLGIW | 348911 | LDKNL | 371708 | HGPLL |
| 255741 | PAAQP | 310405 | GASFY | 348912 | RSCLW | 371711 | IEEDE |
| 255746 | PYKCE | 310406 | RAAEE | 348915 | DQVCT | 371715 | SPVGR |
| 255759 | QAVNR | 310431 | ASMPA | 348917 | EQERL | 371724 | PRSQK |
| 255764 | DHPPS | 310435 | TIHVL | 348918 | LPEKY | 371734 | RGSFD |
| 255784 | SQSEL | 310440 | NLRRD | 348924 | YTSSK | 371735 | MQQKV |
| 255830 | QSTEL | 310447 | LESMV | 348931 | FFNYM | 371741 | PLVSG |
| 255845 | EPAGY | 310448 | NTITK | 348933 | SDEFM | 371747 | CPNIF |
| 255858 | PSPTQ | 310459 | LVFPV | 348936 | RRSQQ | 371750 | LTAVF |
| 255882 | NDIPY | 310465 | RSLAV | 348941 | PSPPS | 371757 | YASVI |
| 255890 | RFPAP | 310471 | RYSAF | 348944 | SAGPA | 371760 | PVRGE |
| 255896 | FMTNQ | 310472 | ETQLI | 348959 | NPAVR | 371763 | ACWWL |
| 255945 | LFAED | 310488 | ADSCP | 348960 | RDQEN | 371767 | ESDHN |
| 255977 | YDLDL | 310490 | LDTTC | 348964 | AVYYC | 371774 | PVRGE |
| 255992 | RWGAH | 310491 | DPSGL | 348965 | VLCTE | 371780 | ESGSK |
| 255998 | RPGRD | 310492 | CQCKI | 348974 | GKTNS | 371783 | SRTQL |
| 256001 | FGVMS | 310493 | EVLQS | 348978 | VRRYQ | 371785 | AQFSK |
| 256010 | DSYYY | 310505 | RSSHL | 348982 | GPARH | 371786 | TFSKA |
| 256015 | MTVSG | 310515 | RCRRH | 348984 | FLNRY | 371787 | MTLMG |
| 256031 | IITIT | 310520 | GKGKK | 348987 | PRRGG | 371789 | GVAGH |
| 256062 | EKDQT | 310521 | PDYDV | 348989 | VRMTQ | 371790 | ILSQS |
| 256078 | KCIIM | 310528 | AEALP | 348996 | ATVIG | 371800 | RYSDD |
| 256079 | VPSNN | 310532 | PQKQD | 349000 | SYLYL | 371811 | GKGLF |
| 256104 | VYERA | 310547 | QYLET | 349007 | GKDRG | 371812 | SQKNL |
| 256108 | RDDED | 310551 | SKKNE | 349016 | ESERD | 371813 | TTVTS |
| 256117 | QILNY | 310557 | RVSMI | 349017 | TSSLR | 371818 | AEEGR |
| 256119 | VRASF | 310561 | QIHAE | 349022 | RTQPE | 371824 | HPRDS |
| 256151 | IQEKC | 310565 | PKFMD | 349029 | PQQPA | 371825 | KTQST |
| 256172 | LPWPL | 310568 | LEDRN | 349041 | AASGR | 371829 | PRSQK |
| 256178 | LEAEV | 310572 | KKLWK | 349052 | MMDEL | 371835 | VSPTG |
| 256183 | TALTN | 310573 | KSQSN | 349057 | NTTLE | 371838 | HFRWN |
| 256186 | QLSRT | 310585 | PTQAP | 349071 | PLLKH | 371846 | EKVTV |
| 256190 | CISDA | 310590 | LSYRR | 349076 | SDKPL | 371847 | ALTHN |
| 256194 | HPLLG | 310593 | FAKVF | 349078 | YKRRK | 371856 | HGPLL |
| 256196 | HCVIF | 310594 | PHCGE | 349085 | EEEGV | 371860 | SHLLE |
| 256216 | DYAKL | 310596 | TLDEY | 349087 | SEVGS | 371872 | GKVEE |
| 256246 | FHPGH | 310620 | LKGEP | 349096 | EMTAS | 371873 | DQVCT |
| 256255 | GTRRR | 310621 | KEKPE | 349101 | GRSRY | 371875 | AAGTQ |
| 256257 | LDELV | 310622 | KKGKK | 349109 | PFTDV | 371877 | IQRRD |
| 256261 | QGLEA | 310623 | EAEKK | 349112 | SCEEL | 371882 | QIMKA |
| 256319 | QKKRN | 310631 | NEMGA | 349114 | AERTC | 371883 | VKAVF |
| 256324 | TTAQV | 310632 | PTALP | 349116 | ISQSE | 371886 | QRRPH |
| 256343 | SKSEE | 310643 | TGRMM | 349128 | LTLRS | 371890 | WTITY |
| 256362 | TLVDG | 310658 | LRPYK | 349140 | KALLQ | 371897 | PLGII |
| 256365 | KTKTT | 310663 | LRKTS | 349142 | AAALD | 371900 | YESSL |
| 256366 | YRQQL | 310668 | EVLMN | 349143 | THQSL | 371910 | LGSED |
| 256367 | EKEAM | 310670 | PAPSN | 349145 | WDTTI | 371911 | KRDHP |
| 256379 | RMRLQ | 310686 | PYNHV | 349147 | TPLIT | 371914 | KRDHP |
| 256383 | AKAED | 310688 | EDETS | 349149 | TEERP | 371917 | DHSPA |
| 256389 | EDEEG | 310704 | FPQNC | 349161 | FISLL | 371922 | VGGSG |
| 256398 | VKMLK | 310721 | YKVKS | 349162 | SSHRD | 371923 | DDLDF |
| 256404 | EIAAC | 310722 | KAELG | 349163 | ISLGL | 371927 | LQQTH |
| 256409 | VVPPM | 310723 | ETIFA | 349164 | VGLSK | 371928 | SKVSR |
| 256412 | NHTTE | 310726 | TKKWS | 349170 | FLARP | 371929 | CLWNT |
| 256425 | ANKIL | 310729 | PQVAN | 349174 | EIELE | 371932 | SKCCQ |
| 256429 | SGDEA | 310733 | AVRRN | 349175 | LDPFP | 371936 | NYDEY |
| 256433 | LLLFG | 310749 | SASQF | 349185 | IRRMN | 371940 | SAGPS |
| 256441 | RGGPE | 310757 | AKFDS | 349194 | PAHWK | 371942 | LRLCT |
| 256442 | AVAKV | 310771 | TRVAL | 349197 | PPKPM | 371944 | QQSRR |
| 256443 | KKLIF | 310781 | SADEF | 349204 | GCLPI | 371954 | RRHGH |
| 256447 | KYQHI | 310785 | HSAAS | 349205 | VKVWK | 371960 | PGVQG |
| 256452 | EDSVF | 310788 | KHFLC | 349208 | NLFFL | 371968 | ESGTF |
| 256458 | ELFGP | 310796 | KSTFI | 349212 | MELTM | 371969 | HLTAT |
| 256460 | LPHQL | 310800 | SETSV | 349238 | PSLSQ | 371973 | RMRPY |
| 256463 | SFLSP | 310801 | SHIKL | 349250 | QKKKR | 371976 | DCFLY |
| 256474 | QRMGD | 310803 | KVLPV | 349252 | GGGKD | 371982 | TMKRY |
| 256495 | LETKD | 310814 | HNSHS | 349266 | NLHDA | 371990 | KIINI |
| 256496 | KSRRS | 310819 | RAFKR | 349270 | TEKYR | 371991 | AQEYV |
| 256497 | MVGLI | 310826 | VATAN | 349274 | SVVKV | 371994 | KERKK |
| 256509 | FPLRA | 310829 | GNPSF | 349291 | PGVSG | 371998 | TTPVC |
| 256523 | AVQRQ | 310831 | GEPYL | 349293 | WPPRG | 371999 | LLRRF |
| 256538 | YIYNF | 310832 | SAVVD | 349297 | NPLGF | 372005 | GQAKK |
| 256544 | LLQLH | 310841 | QSLTS | 349298 | NLTVI | 372010 | SSISW |
| 256545 | AGKRR | 310842 | KKRKK | 349321 | AFYGV | 372018 | KPNLK |
| 256585 | CKYRP | 310856 | FAWKR | 349324 | LIVFV | 372023 | CAAVL |
| 256592 | VGFSV | 310860 | DQAVS | 349325 | SRRSQ | 372032 | PLHLQ |
| 256594 | NKPVC | 310861 | TFSFR | 349329 | KPELE | 372033 | HGPLL |
| 256637 | EDLLE | 310873 | NKIEN | 349330 | GQGTV | 372035 | QPVYV |
| 256644 | HKMAS | 310874 | SLTEH | 349332 | VENGP | 372043 | VSVPK |
| 256646 | MQVYA | 310878 | FTTCL | 349336 | ISTDV | 372047 | AESCI |
| 256649 | RTVAL | 310880 | RWKSS | 349344 | AKVSV | 372051 | PWSCP |
| 256652 | EDEGN | 310897 | TFWEI | 349351 | SIYCD | 372053 | QAIPI |
| 256654 | YLMLK | 310901 | LVHAQ | 349356 | FGCGK | 372059 | DADYD |
| 256658 | EEIKS | 310928 | YKDMY | 349359 | LAGAP | 372062 | VVTPC |
| 256680 | KSKKK | 310935 | RRTEL | 349364 | DSSMR | 372064 | RLFWE |
| 256682 | LKNKK | 310951 | KRMSK | 349365 | LSPQP | 372067 | DYKMR |
| 256686 | NSTAI | 310966 | QQQPV | 349375 | LEFSA | 372073 | DSDLE |
| 256689 | PGGGH | 310973 | PPQRE | 349378 | HGAPN | 372083 | TAEAE |
| 256707 | RESIL | 310978 | LRLQP | 349380 | TKGSP | 372086 | THRSR |
| 256722 | SFSEP | 310998 | NPFKD | 349391 | LVIDN | 372088 | GDAKD |
| 256733 | LPEKY | 311010 | QEDKL | 349393 | NQYLI | 372089 | KSSLV |
| 256759 | SILEW | 311028 | RGRRL | 349396 | RIKVE | 372090 | KGQKL |
| 256797 | GATGR | 311030 | AEVRL | 349401 | PYKCK | 372092 | VSVPK |
| 256832 | FEGVD | 311032 | LYFYH | 349402 | AENEF | 372093 | KQRQM |
| 256854 | QRCTP | 311034 | FNGRV | 349403 | VRERR | 372095 | ECYFR |
| 256857 | QLNQQ | 311035 | DLHTL | 349408 | EEHPL | 372100 | QGRTR |
| 256861 | VHEGQ | 311037 | GSLEI | 349413 | KHKED | 372101 | LLVCQ |
| 256897 | LVESL | 311038 | IQRKT | 349425 | LELSA | 372102 | KPNLK |
| 256906 | RSVSS | 311042 | EEEVA | 349428 | SKSTI | 372105 | DSSRY |
| 256925 | LVQSS | 311080 | LVLKQ | 349430 | YTSAK | 372112 | SASRF |
| 256935 | LSTDL | 311083 | KKPKK | 349433 | LWEGI | 372122 | IIHNA |
| 256951 | VLRKK | 311113 | DHMLA | 349434 | SAPHR | 372123 | PICCY |
| 256953 | TKISS | 311117 | KKYGI | 349436 | SSLYL | 372125 | RPGHF |
| 256958 | SETHC | 311121 | SKKSV | 349437 | DLLHI | 372126 | WAFCC |
| 256969 | SLLNW | 311122 | SPGAP | 349439 | ENRTD | 372135 | PAGRC |
| 256972 | GQSPQ | 311127 | AKGGA | 349440 | QSSHA | 372136 | WAFCC |
| 256991 | SESQV | 311131 | VIFPQ | 349446 | LNPPK | 372137 | ALGMD |
| 256996 | ARTRK | 311134 | DYEEL | 349449 | QNWWV | 372138 | TQREA |
| 256997 | GEDHA | 311135 | PTTVH | 349456 | SSPCG | 372147 | QSYNT |
| 256999 | LSEVA | 311151 | HGALI | 349459 | HDGKC | 372150 | LTKGF |
| 257013 | SCVLA | 311153 | PSSDF | 349465 | SWCDS | 372155 | LQARG |
| 257017 | TSCPC | 311165 | EMKPK | 349467 | MMTAK | 372156 | DEDVL |
| 257020 | DRWRH | 311179 | SDPCE | 349478 | IVEWH | 372160 | SSLIQ |
| 257068 | QADAL | 311181 | YCLLL | 349486 | GPVPS | 372163 | SQKSL |
| 257118 | MLKDS | 311183 | GRAAP | 349490 | QLGAR | 372165 | SQRPW |
| 257177 | GNVSE | 311184 | FLGLH | 349493 | ACTGC | 372170 | MYHLT |
| 257181 | RRGNE | 311196 | GEPTF | 349494 | EILQV | 372171 | WNESC |
| 257189 | CSRLI | 311202 | IVFKD | 349496 | TVISL | 372181 | MWVGY |
| 257192 | VQYSK | 311218 | AFKLK | 349503 | TIACS | 372183 | MLPGH |
| 257198 | TCIKK | 311219 | KIVSH | 349518 | TGKKH | 372186 | PAECS |
| 257209 | SELQL | 311221 | NLGYV | 349528 | ECGKA | 372191 | DGPDK |
| 257215 | VISAR | 311224 | IPSKV | 349529 | SQIPS | 372192 | PEMNK |
| 257245 | SSGPA | 311245 | AVKAT | 349531 | SLHGH | 372193 | SLAQT |
| 257248 | NFTQY | 311246 | KHSIL | 349534 | EKKKM | 372197 | KLASP |
| 257254 | TLVVD | 311257 | SVTSV | 349541 | KSDSD | 372198 | IQFSG |
| 257262 | VGIYV | 311270 | LRPIF | 349542 | SYSVS | 372199 | AASDA |
| 257264 | RWSKY | 311273 | CQLNS | 349543 | EVNAL | 372201 | ITLLG |
| 257267 | SEMEV | 311280 | EPHDI | 349546 | QIVQS | 372202 | LDCGI |
| 257287 | EHRNV | 311290 | SKSWN | 349547 | ACPLG | 372207 | DSSRY |
| 257290 | EDSFL | 311291 | AAGLI | 349550 | IGWNI | 372210 | SRART |
| 257302 | PRENR | 311300 | VSCPP | 349554 | SSHAN | 372218 | RVCSP |
| 257312 | DTSDV | 311304 | PRSGP | 349560 | QPCGC | 372220 | VDCRV |
| 257336 | YDGND | 311307 | SSEES | 349563 | KNERW | 372221 | KFCSP |
| 257347 | QKSAG | 311313 | NCVQM | 349569 | HKMSH | 372224 | LVAPS |
| 257359 | QLCPL | 311318 | DFVFL | 349571 | KPTAF | 372226 | LINTR |
| 257408 | KRVVS | 311319 | RRKAL | 349575 | ARMCH | 372229 | RGSHI |
| 257414 | NDACT | 311320 | KRHGF | 349576 | SQKLQ | 372232 | SQKSL |
| 257430 | LVTSV | 311330 | AQAGS | 349588 | EDNNE | 372233 | SLHSA |
| 257435 | YLHFF | 311336 | PKESQ | 349592 | RTYLP | 372234 | SQRPW |
| 257452 | IMNGH | 311343 | KKNKK | 349609 | MAGDR | 372235 | GLLIK |
| 257497 | LCGGN | 311344 | SQSTD | 349612 | KKHAK | 372239 | SQPPK |
| 257504 | RNHTL | 311360 | SEGEG | 349616 | EEDAL | 372243 | VSSDN |
| 257515 | RETVL | 311364 | GRVCP | 349629 | YQTRY | 372248 | LKKSR |
| 257548 | CKSEE | 311368 | HLYPL | 349640 | KITPQ | 372249 | SDLFL |
| 257549 | NRLPK | 311379 | PFFRN | 349648 | RIHSG | 372255 | GHRPP |
| 257552 | TNGYH | 311390 | SLCIT | 349651 | LPQPL | 372265 | LKETS |
| 257555 | ASSSQ | 311392 | EKLFK | 349658 | CSQAS | 372269 | GASSS |
| 257566 | RSASP | 311398 | LDKVV | 349669 | RRRLV | 372272 | FSAFY |
| 257570 | LFPAS | 311399 | KLCRI | 349677 | HDVYI | 372273 | LWCHI |
| 257572 | GRRNL | 311401 | KTGMT | 349683 | QVRIE | 372275 | SKKCH |
| 257575 | ESAGV | 311402 | MPMPV | 349687 | SLKGI | 372276 | GGVWI |
| 257600 | AAAKA | 311405 | GGPSS | 349689 | AEAEK | 372278 | SYSAR |
| 257604 | EEEEE | 311420 | GSPVL | 349693 | SVKTK | 372281 | PRKKF |
| 257626 | MLLGL | 311427 | MYDNL | 349705 | AAYFV | 372284 | YVYFL |
| 257627 | EMVHS | 311429 | QVHFK | 349708 | VKEKE | 372286 | QLPSK |
| 257632 | MGRWE | 311430 | KKPAA | 349709 | PLRTS | 372288 | LRRTC |
| 257637 | HDVGR | 311436 | ITLLK | 349713 | PASAS | 372291 | LHLYS |
| 257663 | FFVRD | 311447 | FAPDP | 349719 | CSVKM | 372293 | EGKWL |
| 257665 | HTRKK | 311449 | GGCSC | 349723 | QAERS | 372294 | SRSVR |
| 257694 | FFSKL | 311452 | RIHTH | 349727 | PCDRP | 372295 | SKKPV |
| 257696 | PSRSM | 311453 | RPPPE | 349732 | NAIHA | 372300 | QGPCL |
| 257700 | PNTGK | 311463 | GLGSS | 349734 | RIAAT | 372302 | GWEVR |
| 257704 | KYLLQ | 311477 | AKLLT | 349748 | KKPRF | 372305 | EHTLF |
| 257724 | ICFPS | 311479 | QPASP | 349757 | LQWWK | 372306 | LESTL |
| 257745 | GSGWH | 311486 | LLQRK | 349768 | DEVQI | 372310 | KAMNM |
| 257749 | RKDYT | 311489 | FKKNK | 349770 | PAGMQ | 372313 | ASTLA |
| 257766 | NAPEY | 311492 | CATST | 349781 | SRTKM | 372315 | FKIGM |
| 257770 | FVLYQ | 311493 | GDAEW | 349783 | KMVNS | 372316 | PSFCL |
| 257776 | HKDLD | 311496 | QSQVV | 349785 | PCNPL | 372319 | KIDEF |
| 257787 | ASYVS | 311500 | TQGST | 349790 | KGPDP | 372320 | STTRV |
| 257789 | TWGGC | 311502 | RRDYF | 349794 | RRRWT | 372322 | DEDTD |
| 257818 | INGMI | 311505 | GTKPP | 349796 | SEGKS | 372326 | TSQQL |
| 257821 | GEETK | 311508 | SQISS | 349802 | PACFL | 372328 | CVPLP |
| 257829 | LKRKK | 311513 | NSEQL | 349805 | TAVWV | 372331 | WPAGH |
| 257831 | RENEN | 311521 | SRQKP | 349808 | ALFVL | 372335 | MTNVS |
| 257836 | SLPSH | 311538 | GDEGS | 349823 | FTSPL | 372339 | SKLKT |
| 257857 | GYEVM | 311540 | TGERP | 349824 | PVTVL | 372342 | NDDKN |
| 257860 | SAHSY | 311545 | ASKTG | 349828 | KLVKD | 372353 | CSCSH |
| 257861 | EKGLF | 311550 | HHHRG | 349839 | AQVFE | 372354 | EITCF |
| 257863 | TLSPV | 311570 | CSRPT | 349841 | GPPLE | 372358 | RGARP |
| 257867 | LKPWA | 311572 | TIVKA | 349853 | SCPVG | 372368 | RSPKM |
| 257868 | RCGCS | 311579 | AEQKT | 349855 | VLLGV | 372377 | CADSF |
| 257894 | LTLDV | 311581 | SETVV | 349860 | SVTVE | 372381 | GLLPT |
| 257895 | AQAVY | 311592 | KVHGF | 349861 | RSIKV | 372382 | CADSF |
| 257899 | QSAPS | 311596 | TRENI | 349873 | KAKGK | 372385 | LNRSG |
| 257901 | SVRFA | 311605 | NPGRE | 349874 | SSLPA | 372389 | VFFLV |
| 257904 | EGNPE | 311609 | YLQYQ | 349877 | LKVPS | 372390 | PGHME |
| 257905 | GANSV | 311642 | ACQLT | 349882 | ALSTS | 372394 | VCTVR |
| 257909 | PQGSP | 311648 | EKKVK | 349883 | QWFTG | 372398 | SIHPA |
| 257910 | PSAFL | 311656 | HYSSL | 349884 | DVLKS | 372404 | RLREP |
| 257915 | HIILK | 311657 | VPGHR | 349887 | PPSAQ | 372408 | PRRRR |
| 257927 | EVHQT | 311665 | QLSDL | 349891 | SRKKI | 372410 | LYSGI |
| 257934 | PVSLR | 311677 | PSLLI | 349892 | NAHTF | 372412 | REITE |
| 257935 | GAVNV | 311679 | FHSAE | 349893 | KTTAL | 372414 | LNVKI |
| 257940 | TAMPH | 311682 | QNIFA | 349909 | PQLLK | 372420 | QDWKD |
| 257951 | CRTKY | 311684 | YPGGP | 349923 | PPVSR | 372422 | AVGDS |
| 257963 | EDVKI | 311687 | FIIHS | 349929 | EEEDS | 372424 | AFYGF |
| 257974 | PSHKH | 311688 | KISSL | 349930 | KFWGT | 372433 | GMECN |
| 257979 | NTQAL | 311695 | CSPDD | 349931 | SHCLC | 372445 | GSGSL |
| 257981 | EGTGV | 311697 | KFRFG | 349942 | GMVPK | 372456 | LRTLP |
| 258034 | DLYQE | 311698 | LAERE | 349945 | LDSVF | 372459 | QKLTA |
| 258042 | QEMAL | 311712 | FRSSV | 349954 | PILPQ | 372460 | LGYVK |
| 258052 | RTKEQ | 311713 | QLSIS | 349955 | IYSED | 372465 | YNAIS |
| 258070 | ETFGR | 311740 | AKPKV | 349957 | AGGAS | 372468 | GLLPT |
| 258080 | PEVTE | 311746 | VIRSN | 349959 | TSAES | 372475 | SQLLS |
| 258081 | AQEAD | 311747 | YQRRM | 349960 | HRKCF | 372476 | RFSRR |
| 258083 | PWTFW | 311750 | RLTQS | 349962 | EPAAA | 372477 | RFSRR |
| 258091 | RGRPH | 311760 | RDVIG | 349967 | HSTEV | 372479 | LYYVN |
| 258092 | SFASL | 311766 | GHPLL | 349971 | AEVAS | 372482 | KLRQK |
| 258105 | ADTGR | 311768 | ADVQA | 349977 | VPVLR | 372483 | PHVFI |
| 258106 | VTSND | 311775 | MWQEI | 349989 | KRTRS | 372490 | GRSWK |
| 258111 | KRKFS | 311778 | LFENN | 349996 | HFLGK | 372492 | TKKET |
| 258145 | SKHLL | 311792 | KKKRD | 349999 | FRCSP | 372501 | IITFQ |
| 258168 | GAPLT | 311803 | ARQAG | 350003 | LSIVF | 372503 | GANAH |
| 258169 | LKPQD | 311816 | AQGQE | 350005 | RLRSR | 372505 | QEEVD |
| 258180 | QMATE | 311825 | HPNEQ | 350009 | ETRSL | 372508 | TCFLR |
| 258198 | TENEA | 311827 | MRFDC | 350011 | LQLLS | 372510 | KAGGA |
| 258200 | PTLVA | 311833 | VLTHQ | 350012 | MYGRK | 372522 | LKYEK |
| 258201 | PGLEV | 311837 | YMYSS | 350014 | KIMTC | 372524 | ATKLG |
| 258214 | QIQVA | 311842 | HVVLS | 350017 | WGSRR | 372527 | CKTLY |
| 258229 | VSDEQ | 311847 | HVISS | 350018 | ASETN | 372528 | DIFFI |
| 258243 | KRYTA | 311856 | KKLDL | 350022 | GRQSS | 372531 | CHTDT |
| 258281 | ENQEH | 311862 | TQTAN | 350024 | FKALL | 372534 | PGCGD |
| 258301 | LFLVL | 311866 | HLKLY | 350028 | WRNEL | 372539 | LSDSD |
| 258302 | QRRLS | 311873 | RAIFQ | 350031 | SCSKG | 372541 | GGGAN |
| 258317 | LEAGS | 311876 | KPYSD | 350032 | QSTDE | 372542 | FEHAT |
| 258324 | SRFLS | 311888 | DPSWN | 350036 | HGESR | 372547 | HWTKL |
| 258341 | SIEKP | 311899 | CRIWR | 350037 | FRAGL | 372550 | ARALF |
| 258349 | PSSAP | 311921 | PLRLQ | 350041 | SKNFP | 372551 | APGKA |
| 258380 | TISRG | 311925 | VLKSC | 350042 | NLLTS | 372552 | DRFVR |
| 258381 | FWTLP | 311951 | KGKRK | 350049 | VIIQD | 372553 | ADLSE |
| 258383 | SITAS | 311957 | IYLEP | 350050 | HVKTD | 372554 | VSKKK |
| 258385 | DKRFI | 311962 | VLGAA | 350052 | LVIDN | 372556 | HKPNF |
| 258390 | SSAEV | 311977 | ANAYK | 350054 | SDLCR | 372557 | TKDPK |
| 258398 | LAVSP | 311984 | LQQAL | 350062 | SPVFA | 372558 | GGLFM |
| 258399 | TRQAL | 311990 | YCFVI | 350066 | LCEVW | 372559 | IAHLE |
| 258400 | QVSYV | 311997 | AAKGK | 350071 | AKVFV | 372563 | PHQLA |
| 258403 | MSTKL | 312001 | PTNMS | 350075 | EEWDD | 372565 | VCFMG |
| 258405 | QINKP | 312002 | RFAMP | 350085 | EKPYK | 372570 | ALLGP |
| 258411 | VSVCK | 312008 | EKKKR | 350092 | PPKPM | 372573 | LAIGR |
| 258412 | MGDRN | 312017 | AKKDG | 350094 | ASTMH | 372576 | CENDG |
| 258415 | LQRQC | 312021 | TLAKP | 350095 | NLHVP | 372582 | QRGSA |
| 258417 | NGYPV | 312027 | PNQYQ | 350098 | AEAME | 372585 | AQGTL |
| 258418 | AQQEA | 312029 | REAPF | 350102 | TVNLL | 372587 | SSVPR |
| 258424 | QQLAH | 312031 | GLLEK | 350103 | VFTEP | 372589 | MEGLL |
| 258428 | TLKVT | 312042 | WGPKR | 350107 | AKAIG | 372592 | PCSGD |
| 258436 | KLKSE | 312050 | ETNIA | 350111 | RRTRC | 372593 | GWHIS |
| 258439 | PAYTP | 312054 | QHETH | 350121 | KIMTC | 372595 | KFYDE |
| 258449 | PGTRT | 312059 | TNPFL | 350125 | CRIIS | 372596 | ADAPE |
| 258455 | TWKKR | 312066 | FGFRS | 350132 | IVMLL | 372597 | REANQ |
| 258456 | NQSAV | 312074 | IRLLA | 350136 | KLLVD | 372599 | KLAQL |
| 258457 | HVTWP | 312076 | KAERE | 350137 | FCILL | 372601 | HQGLL |
| 258459 | QVFES | 312081 | EEGEA | 350140 | ESKHN | 372602 | KGLLH |
| 258484 | AMEVT | 312082 | SIKQY | 350147 | DKLGQ | 372606 | ELKCR |
| 258494 | VTLEY | 312099 | RRIVE | 350159 | YGFGG | 372607 | TGFLS |
| 258499 | NEILS | 312107 | ELSEH | 350162 | RNANV | 372608 | RRGPL |
| 258526 | KCKWM | 312126 | SATLG | 350170 | LNGVS | 372611 | KKVEE |
| 258530 | AESEA | 312129 | PIMHM | 350175 | KGGKG | 372612 | LLKFI |
| 258531 | TPMES | 312134 | FICSY | 350178 | TTVGQ | 372614 | PTHTC |
| 258534 | INGDI | 312141 | SETIL | 350182 | EVPSS | 372617 | SDTSN |
| 258589 | NQNMQ | 312142 | CVVSQ | 350183 | EDDKC | 372622 | PQPPE |
| 258597 | GLCDR | 312143 | SPKKV | 350194 | AHLSH | 372626 | STGGP |
| 258603 | SVCPS | 312150 | SSFFC | 350195 | VTSGP | 372633 | WLLSI |
| 258613 | EKLVI | 312185 | VYDCN | 350198 | LQTSI | 372634 | PGGGG |
| 258646 | GAFKN | 312189 | VHSSS | 350199 | TILKN | 372635 | ENVVL |
| 258648 | SSESD | 312205 | YIFSV | 350202 | AVTSH | 372637 | EPWLS |
| 258654 | LLVSK | 312206 | QRYSR | 350204 | VSALS | 372640 | NYWDP |
| 258662 | KGNHL | 312208 | ISRSC | 350215 | CFPNS | 372645 | PAGGG |
| 258679 | ILDTL | 312213 | RNAKQ | 350216 | HLLDL | 372648 | QGCQV |
| 258682 | VAPLQ | 312222 | QLETD | 350220 | RRTEE | 372649 | RLFQE |
| 258704 | RARLS | 312224 | HLIEF | 350222 | SQGGK | 372650 | RLFQE |
| 258711 | NLLRD | 312235 | PRPDY | 350228 | IYTYP | 372651 | LHCVY |
| 258729 | QSRRK | 312242 | EDDRD | 350230 | ILATL | 372653 | RLPET |
| 258738 | AQSVQ | 312244 | PRAIY | 350236 | EQRPL | 372654 | VLSSV |
| 258739 | LSLPA | 312246 | TADLA | 350240 | GLQLE | 372657 | TPSPK |
| 258742 | ELLNV | 312250 | VHPHY | 350249 | AEDVI | 372658 | SLYTQ |
| 258743 | ALRQM | 312262 | SANGL | 350256 | GALSL | 372660 | LVDGF |
| 258749 | DQGGH | \|312272 | DNGWD | 350260 | SCTLQ | 372669 | KKRKK |
| 258761 | EGEEN | 312273 | IDLSA | 350261 | RTSTI | 372670 | VYGTL |
| 258767 | NRSKE | 312277 | ALNTN | 350263 | KSHFM | 372671 | KMPEN |
| 258770 | EELAK | 312284 | LLKVY | 350265 | FETTI | 372676 | IEEVD |
| 258772 | TAKPS | 312286 | KYCQS | 350266 | EKTVK | 372678 | QANRK |
| 258774 | IPALS | 312299 | KLGAP | 350267 | TSSYL | 372681 | LSDSD |
| 258775 | MELTM | 312304 | LLTEK | 350273 | FRCSP | 372683 | GGGAN |
| 258776 | CWFHK | 312309 | VLARP | 350275 | RGERA | 372684 | FEHAT |
| 258780 | VELIE | 312311 | FVSDL | 350278 | DFAAM | 372687 | GLASW |
| 258787 | LWPSR | 312318 | QQLQH | 350283 | PHSHY | 372689 | GMPMF |
| 258796 | SSGSH | 312323 | GGTFP | 350285 | DRVSF | 372691 | ARALF |
| 258798 | TKVSK | 312326 | GFSHN | 350295 | ETLQM | 372694 | APGKA |
| 258807 | RLHSY | 312350 | SRQPL | 350311 | VWRMH | 372695 | DRFVR |
| 258821 | RPQCE | 312355 | SSDIC | 350313 | PQPPE | 372696 | AWLQS |
| 258829 | VSWNW | 312356 | KKAKT | 350314 | TFIPI | 372697 | HKPNF |
| 258869 | TLLAL | 312367 | YEIVI | 350316 | LLFSR | 372699 | TKDPK |
| 258873 | QEQKM | 312368 | KEEDL | 350331 | YLSYN | 372700 | IAHLE |
| 258874 | DSSTA | 312370 | RQSPP | 350332 | VRGPQ | 372703 | PHQLA |
| 258883 | RVARR | 312381 | ASHPS | 350336 | IQFMP | 372706 | VCFMG |
| 258884 | ELPNS | 312385 | APLTR | 350352 | GPGID | 372711 | AQGTL |
| 258886 | ARKFS | 312397 | IHKSL | 350358 | QGGFI | 372713 | SSVPR |
| 258888 | AQGMR | 312399 | KKVTM | 350359 | LNPIL | 372717 | PCSGD |
| 258930 | FHIRI | 312402 | PWGGH | 350364 | NAILQ | 372718 | GWHIS |
| 258947 | FCHSL | 312403 | GRDSG | 350369 | TDARS | 372720 | ERKSS |
| 258955 | PVPGG | 312411 | SYKIE | 350377 | KRSAL | 372721 | KFYDE |
| 258960 | GLVLQ | 312415 | QEDDE | 350386 | EESSE | 372722 | ADAPE |
| 258962 | SRSRT | 312419 | VAQQQ | 350387 | FTWTL | 372723 | REANQ |
| 258963 | TSPPW | 312435 | PYVPP | 350389 | TFFNI | 372726 | KLAQL |
| 258969 | KAGRH | 312436 | MEATD | 350392 | MELTF | 372734 | KGLLH |
| 258975 | YDNIE | 312439 | LNVYL | 350402 | VSGKV | 372735 | ELSNN |
| 258991 | AADQP | 312442 | VESWQ | 350408 | HSKET | 372738 | HQGPL |
| 259006 | GTKTA | 312455 | ILSYT | 350409 | KYRVK | 372741 | FADDP |
| 259008 | FPGFK | 312458 | KSQDF | 350415 | TDLQI | 372746 | RRGPL |
| 259021 | PPKGT | 312464 | KAHES | 350418 | CAGEK | 372751 | GEGKK |
| 259030 | CFTSE | 312470 | VLHTI | 350420 | LYQVD | 372752 | PTHTC |
| 259034 | ERKCP | 312506 | GQYWV | 350422 | QFVLA | 372754 | LPPPR |
| 259037 | ATPDN | 312530 | LQQKD | 350425 | APLES | 372757 | SDTSN |
| 259043 | SPRRY | 312535 | EESVV | 350444 | IRINP | 372760 | IPRTK |
| 259050 | TFDIA | 312550 | YEVSI | 350451 | KFRPY | 372767 | STGGP |
| 259053 | PVQFD | 312576 | IAANS | 350467 | SAEPC | 372771 | ISELP |
| 259055 | SMMHL | 312599 | HKDDK | 350469 | SAKKL | 372773 | PVPGG |
| 259056 | KYYEA | 312606 | FNDPY | 350474 | VLVGK | 372776 | WLLSI |
| 259060 | IQSQI | 312615 | PYFEK | 350479 | SGKKA | 372779 | IKAEI |
| 259075 | FNGET | 312617 | QVTGD | 350482 | ENLGG | 372782 | PGGGG |
| 259089 | YELQP | 312618 | LDRTC | 350491 | LKRDF | 372783 | ENKPT |
| 259119 | KTAKE | 312624 | EAQRG | 350498 | LGLQA | 372785 | GVNHR |
| 259154 | QEYSL | 312625 | FRVKK | 350502 | SYSVS | 372787 | AVMVG |
| 259199 | DQVCT | 312631 | VRPAC | 350504 | ERERL | 372789 | EPWLS |
| 259205 | LNIND | 312649 | QQSLH | 350511 | TGRWK | 372790 | GIIAL |
| 259206 | FQEDE | 312652 | LSPGC | 350512 | QINIS | 372791 | GAFAL |
| 259211 | LTMLF | 312663 | IARAD | 350524 | LIELH | 372795 | NYWDP |
| 259212 | FQQCD | 312664 | GHPPT | 350525 | ARRLR | 372797 | SDSED |
| 259216 | LGHRL | 312671 | RCVLS | 350540 | NVNDE | 372798 | AEALK |
| 259229 | EPGAA | 312673 | GSCGF | 350541 | LRRNT | 372801 | YRISA |
| 259235 | RKEKV | 312675 | EGAEA | 350546 | FNAQV | 372805 | IEQTR |
| 259239 | FLSTE | 312678 | TEQLP | 350547 | DETSI | 372808 | LGGGA |
| 259241 | IIEKW | 312679 | QQHVV | 350549 | LLRPV | 372810 | KGTLL |
| 259250 | MWQEI | 312697 | KAKKP | 350552 | TGWAK | 372812 | PGQSQ |
| 259253 | KREEL | 312700 | PRLQC | 350556 | GRRTF | 372815 | QGCQV |
| 259254 | KEYFI | 312702 | EDDMF | 350558 | SSAYC | 372816 | VSLTA |
| 259271 | LGQDL | 312706 | QHPTT | 350576 | HEMTH | 372817 | RLPET |
| 259289 | TCEIQ | 312709 | SLLEK | 350580 | YTSSK | 372820 | TPSPK |
| 259324 | DKEQA | 312716 | CIALW | 350583 | LQEYL | 372821 | SSSTL |
| 259339 | RLDFH | 312734 | RCSLL | 350592 | REGEE | 372825 | PMHSN |
| 259351 | LMSFE | 312735 | RMMSV | 350607 | EVLLC | 372826 | PCLCL |
| 259357 | NTLGG | 312742 | PTREA | 350611 | EIDMA | 372827 | SLYTQ |
| 259362 | RFFSK | 312753 | EQDRP | 350614 | AQTRL | 372829 | AGIQT |
| 259365 | CRSGV | 312758 | WSQQA | 350626 | KSAGG | 372832 | PQMSS |
| 259396 | EEGES | 312767 | FQNIC | 350630 | LHMKK | 372834 | LVDGF |
| 259400 | DKKCS | 312770 | VHEVF | 350633 | PGPGR | 372835 | LSSLI |
| 259406 | MSPPL | 312773 | GSLDG | 350638 | FWLLP | 372836 | REEYR |
| 259410 | VLPPH | 312774 | PPRLL | 350639 | GCNNQ | 372837 | AGMRR |
| 259455 | MVSGL | 312778 | IGLKP | 350642 | HVQEH | 372839 | RLQWG |
| 259456 | SYVLF | 312779 | AALPQ | 350649 | RTLAP | 372840 | DLGFL |
| 259457 | TMDTS | 312789 | TAASS | 350665 | HKKTH | 372842 | RNSGP |
| 259466 | HRIYS | 312814 | RGSRM | 350667 | ELNNM | 372845 | VVCNT |
| 259467 | DLEID | 312834 | RLGES | 350676 | GKLLL | 372849 | QKHSS |
| 259469 | YAVKA | 312836 | KLDLA | 350681 | DLQKT | 372853 | NFLPL |
| 259470 | SYPNV | 312837 | LEIKH | 350686 | ELAAL | 372856 | EVGPV |
| 259477 | ARKTV | 312864 | HNDWF | 350691 | LSSYF | 372857 | LQNHL |
| 259486 | YESEI | 312939 | ADKTA | 350696 | KRKGL | 372860 | GSPEL |
| 259512 | RLGDQ | 312942 | GDERI | 350698 | KRKGR | 372868 | ALNTV |
| 259526 | TRGKM | 312946 | ILTVK | 350704 | FFLEE | 372870 | KKRKK |
| 259555 | LRRKD | 312952 | LNRHR | 350708 | NFDED | 372871 | HPAAF |
| 259569 | LLNFA | 312970 | GGTNK | 350710 | KSMLQ | 372873 | PLRTA |
| 259605 | HRDSE | 312981 | NQQGA | 350716 | SGPWL | 372874 | KMPEN |
| 259607 | TPKGP | 312988 | DPRPV | 350718 | VLLPS | 372879 | EMSST |
| 259608 | QPAAS | 312995 | KYLES | 350719 | GPPPT | 372881 | IEEVD |
| 259631 | LRKMG | 312999 | DCGLF | 350720 | GSEED | 372883 | LQQKQ |
| 259632 | KPAEE | 313007 | GVPTV | 350734 | GLSSG | 372890 | DGEAE |
| 259633 | FRFPD | 313018 | IALTN | 350736 | ALGLP | 372894 | ESCRR |
| 259667 | QWPPG | 313021 | YGGHR | 350750 | YTSFD | 372896 | AKALK |
| 259698 | VATAF | 313025 | GGLAL | 350767 | YGFGG | 372899 | REELG |
| 259708 | LSSSS | 313026 | FGTQA | 350773 | QPMET | 372900 | SQHNP |
| 259711 | VLCCQ | 313029 | IRDLL | 350776 | LGDSM | 372901 | TRPHS |
| 259726 | LLDSP | 313034 | ASQRR | 350780 | LILYF | 372905 | LWLLH |
| 259727 | NTVFS | 313046 | LQTEV | 350785 | SVVSR | 372908 | LCSKP |
| 259737 | TLQSH | 313049 | ALLNV | 350786 | EDFAC | 372909 | SEITE |
| 259748 | TDLPL | 313050 | TSPLQ | 350787 | FFSHR | 372914 | MPWHL |
| 259750 | RAGAR | 313055 | VGEKP | 350789 | EQFAN | 372916 | STRPW |
| 259782 | SSDEP | 313059 | NLRTT | 350809 | TIASH | 372917 | GLYTP |
| 259791 | KAKGK | 313062 | ADVFL | 350818 | SRRKT | 372920 | PQSAL |
| 259803 | LCPLR | 313079 | PAVTV | 350820 | ISTQT | 372922 | GPPLP |
| 259806 | KPCVM | 313088 | NTTRV | 350823 | KDRTK | 372926 | MNLEF |
| 259808 | YVSQN | 313104 | GRRQI | 350833 | NPQGS | 372929 | SRPRE |
| 259818 | GEDEA | 313110 | EKAVK | 350835 | VEEKK | 372930 | ELSNN |
| 259845 | REEYR | 313121 | VTSWM | 350836 | SFLKE | 372935 | NGPPL |
| 259870 | RLQWG | 313140 | LQWLF | 350844 | AYDRH | 372942 | GEDEC |
| 259873 | PQSAL | 313158 | VLLGI | 350848 | PQSIV | 372943 | ESHSL |
| 259881 | LSSLI | 313159 | AEEEY | 350852 | RRRLH | 372945 | GSSNQ |
| 259882 | EMSST | 313169 | RKNAV | 350856 | VCAEV | 372947 | LPPPR |
| 259883 | AHLSD | 313171 | AADFP | 350864 | EEMSG | 372951 | PTDWE |
| 259895 | QKHSS | 313172 | VHQLP | 350870 | SGKWE | 372953 | YRWGN |
| 259915 | PMHSN | 313199 | SYKPY | 350873 | LWEEP | 372955 | RPYSQ |
| 259925 | AEEEA | 313214 | GMYGR | 350874 | VLKPV | 372957 | LLLRP |
| 259930 | LTLKG | 313225 | QLSLE | 350878 | GIFCN | 372959 | DKKHT |
| 259938 | GRSKQ | 313226 | DYALV | 350881 | MAVES | 372962 | PPQKL |
| 259939 | DPSTT | 313243 | EELKE | 350882 | SLFVN | 372963 | KVPSS |
| 259951 | IKIFD | 313262 | GGSCL | 350885 | SLHGH | 372966 | ISELP |
| 259953 | DGEAE | 313272 | KTSGH | 350894 | MRDEL | 372967 | VPAES |
| 259963 | RNGVR | 313280 | EHFQL | 350896 | PAPQY | 372969 | PVPGG |
| 259969 | MKTKK | 313287 | YGFVS | 350897 | LRLCT | 372971 | EKEDS |
| 259983 | DETPK | 313299 | VEMLG | 350906 | IRQGH | 372978 | ENKPT |
| 259988 | QDTSC | 313309 | LTPLL | 350914 | MIYIR | 372979 | ITCKA |
| 259989 | SFFRG | 313311 | FFQFG | 350928 | LGQDL | 372982 | VHSLR |
| 260008 | NPLRK | 313316 | LFGNP | 350934 | DIKDT | 372984 | AVMVG |
| 260010 | AAIKS | 313318 | SAMGV | 350937 | KKRMS | 372987 | TREPL |
| 260011 | LVHLV | 313324 | RYPLI | 350939 | PSQRA | 372988 | GIIAL |
| 260045 | TVENT | 313346 | PAQEN | 350945 | FISRV | 372990 | TACKV |
| 260054 | YRFWK | 313350 | KQSED | 350946 | YLNKI | 372991 | GAFAL |
| 260056 | CCNFN | 313355 | SFSST | 350951 | GQRQL | 372995 | YRISA |
| 260058 | SSLKM | 313360 | EHGEN | 350958 | QKPIL | 373000 | IEQTR |
| 260061 | QQYKA | 313363 | GKLSV | 350961 | AYRVV | 373001 | MWCMY |
| 260102 | KYYTS | 313377 | LLKIP | 350964 | FSQED | 373002 | RTVYS |
| 260113 | LYWFK | 313380 | GLYTP | 350967 | NKCAL | 373003 | RGSHL |
| 260116 | SESIQ | 313384 | TFFSL | 350969 | QVSLQ | 373004 | LGGGA |
| 260118 | CYIFD | 313385 | RSGTE | 350975 | WDVDG | 373005 | DCGQL |
| 260126 | LEPPS | 313391 | GNPFA | 350976 | SPAFF | 373006 | STEGA |
| 260128 | DGWEG | 313396 | GRFLC | 350990 | KKKKV | 373008 | IKIFD |
| 260129 | PASET | 313399 | TVYFW | 350991 | IRINP | 373009 | ILKHK |
| 260130 | TIPEV | 313408 | DEQDA | 350992 | TLLES | 373015 | KITVW |
| 260147 | QHTEV | 313416 | EDCDT | 351007 | DHFDK | 373016 | PMHSN |
| 260187 | PPSRM | 313419 | TWSTR | 351015 | IKKIV | 373018 | AGIQT |
| 260191 | LWGGV | 313420 | WEPWM | 351028 | SLSPT | 373022 | REEYR |
| 260197 | PMVIA | 313423 | LPALL | 351035 | KMKAQ | 373023 | LLDSP |
| 260210 | SVEDM | 313437 | TTFEY | 351040 | TYKLK | 373024 | RLQWG |
| 260227 | NSRKK | 313441 | AFTPG | 351042 | ETLQM | 373027 | RNSGP |
| 260228 | SWIGC | 313443 | HTINI | 351047 | ELLNA | 373031 | VVCNT |
| 260229 | TSIYQ | 313444 | FLMPA | 351049 | QNRTR | 373034 | HLLYK |
| 260246 | VGDMS | 313454 | SHDTD | 351052 | SLCSC | 373037 | GSPEL |
| 260247 | VRQTS | 313474 | HNVKI | 351056 | DQFLQ | 373038 | GLTQS |
| 260257 | YVIPR | 313490 | LEGSC | 351059 | ALLLL | 373039 | YCIGG |
| 260264 | STYLH | 313500 | CGTSS | 351068 | QRINR | 373041 | VDVET |
| 260270 | VGKTS | 313504 | PCLIS | 351075 | WGAQP | 373042 | SWRAA |
| 260276 | SYSKP | 313508 | ADWLR | 351076 | GRLNW | 373043 | TETLD |
| 260282 | QKAEN | 313510 | LMSCL | 351086 | PDCPG | 373047 | VIRRL |
| 260283 | LTKDI | 313513 | EPAEG | 351100 | GDDSD | 373048 | WQQHH |
| 260309 | LFPGH | 313515 | AVRNP | 351104 | RQRSK | 373054 | HPAAF |
| 260323 | TEESA | 313516 | EADWS | 351105 | SRSGK | 373056 | PLRTA |
| 260324 | HLGMS | 313529 | ILSLV | 351106 | TRVTH | 373057 | FLAQK |
| 260327 | LLPPD | 313553 | AGKTV | 351108 | NYKPY | 373058 | AEEEA |
| 260356 | ECRDP | 313569 | VGGVH | 351114 | LRWGS | 373060 | EMSST |
| 260359 | ILAED | 313571 | SVRIF | 351118 | DDSYI | 373065 | DGEAE |
| 260361 | PRLFK | 313572 | FCSIL | 351126 | TGKKK | 373066 | KCISG |
| 260363 | KRKKP | 313580 | CDPAL | 351132 | LLKQR | 373067 | ESCRR |
| 260372 | TINAK | 313581 | IAANP | 351137 | SLKED | 373071 | REELG |
| 260382 | ITDQK | 313582 | RVHTD | 351138 | LGELQ | 373073 | QALQL |
| 260385 | VILRD | 313600 | SSLTI | 351141 | QGSVL | 373075 | LCSKP |
| 260386 | SLAER | 313601 | QRLRR | 351143 | PFFPS | 373079 | STRPW |
| 260402 | QESRL | 313623 | IFNIF | 351147 | CYSYS | 373080 | GPPLP |
| 260403 | SHLSS | 313643 | EGHHV | 351148 | VSPMH | 373082 | SRPRE |
| 260404 | QTSTC | 313644 | SLLSW | 351149 | NKDVA | 373086 | NGPPL |
| 260408 | GHMRR | 313657 | YINII | 351154 | ECDGL | 373090 | GEDEC |
| 260433 | RCLEH | 313661 | FRKAS | 351157 | QEHVE | 373091 | ESHSL |
| 260435 | EKERS | 313666 | FQKYH | 351163 | VCFLG | 373096 | PTDWE |
| 260442 | WTRLL | 313668 | ARSLS | 351164 | ASLWA | 373102 | LLLRP |
| 260443 | MEDAP | 313670 | SRELW | 351167 | QAGEV | 373105 | PPQKL |
| 260447 | CLHKE | 313681 | PEEPL | 351168 | EWVCI | 373106 | VPAES |
| 260453 | ICEEK | 313691 | ENKQN | 351182 | YIKGF | 373107 | GPMQH |
| 260502 | KQAEL | 313699 | PTDAL | 351190 | FLKRP | 373108 | EKEDS |
| 260505 | RYGHL | 313704 | PALYF | 351197 | SETSL | 373111 | TREPL |
| 260508 | SVQKS | 313731 | RIQRS | 351206 | LEGVD | 373114 | TACKV |
| 260526 | IPQFV | 313738 | IDSEV | 351207 | YENQV | 373119 | MWCMY |
| 260536 | IRRMN | 313744 | TGGVY | 351209 | VGIPI | 373120 | RGSHL |
| 260563 | TNPNL | 313752 | GGRDS | 351211 | SKRPF | 373123 | IKIFD |
| 260570 | SFSFQ | 313759 | LASSC | 351214 | SLLWG | 373124 | ILKHK |
| 260595 | ENDEI | 313764 | NSHGL | 351218 | PGHVD | 373129 | KITVW |
| 260598 | FDGIV | 313775 | LNLTL | 351228 | TRELT | 373134 | HLLYK |
| 260600 | VVDNS | 313809 | HFQEP | 351238 | CGQLE | 373136 | GLTQS |
| 260604 | SSNSQ | 313811 | ETRAV | 351244 | DFRKM | 373137 | YCIGG |
| 260605 | IELDS | 313816 | EVLQE | 351245 | LKNGT | 373139 | VDVET |
| 260619 | HSSGL | 313822 | SSSLH | 351248 | SVKWN | 373140 | SWRAA |
| 260630 | KETCQ | 313823 | RTGWS | 351249 | STGEK | 373141 | TETLD |
| 260637 | SYCQG | 313829 | PYGRY | 351250 | PVHIK | 373143 | VPMEI |
| 260643 | FPGFL | 313833 | SKEAP | 351254 | RRAEM | 373147 | LMGWE |
| 260645 | HLISR | 313852 | PRRRR | 351255 | PYSAE | 373148 | WEWGK |
| 260648 | KYLKF | 313854 | APHTD | 351258 | GDNQE | 373149 | FFCKK |
| 260649 | LYTSC | 313859 | RSQEP | 351268 | WKMVP | 373150 | RNCKS |
| 260653 | SECDV | 313869 | ISTTE | 351273 | VFPSD | 373151 | ILTSA |
| 260662 | MSLVS | 313873 | LTIGA | 351280 | RDRPG | 373152 | FLAQK |
| 260665 | RENSS | 313875 | AEQRG | 351283 | LTLKG | 373153 | HKKFE |
| 260682 | CFIPV | 313877 | AAETE | 351284 | KYDPS | 373156 | ITFTA |
| 260702 | RNNLI | 313880 | AEEGD | 351292 | RSPYK | 373169 | KILSC |
| 260731 | AQINL | 313881 | FNLIL | 351298 | PPKKR | 373184 | CHCSS |
| 260742 | HKWKS | 313886 | KWVCW | 351310 | AANWS | 373191 | EKGPE |
| 260743 | ALLPS | 313890 | VRDTA | 351314 | AMEVE | 373193 | ILMKK |
| 260746 | NAKKK | 313921 | VGIKK | 351318 | SSSFA | 373194 | SPLAT |
| 260762 | MSQHM | 313933 | SILEC | 351320 | PIDST | 373195 | VKVSY |
| 260803 | DDDAA | 313936 | WLPRV | 351325 | LPEGV | 373197 | IIPKI |
| 260810 | RPRVH | 313947 | STHLR | 351327 | LLANL | 373200 | KEYFI |
| 260818 | LGYQT | 313950 | LQSVA | 351335 | RRFSV | 373203 | RSSYR |
| 260843 | DYTDV | 313953 | FRNQY | 351342 | HQSPE | 373206 | KHDEL |
| 260867 | LQQSL | 313967 | PDMEA | 351345 | LCSQT | 373214 | DEEDD |
| 260869 | IKGSR | 313973 | VKNCS | 351346 | RKGFP | 373226 | EGIWA |
| 260926 | EIDQR | 313983 | EEVKE | 351348 | NLLTL | 373228 | GALGF |
| 260947 | LPLDS | 314004 | PKNCS | 351352 | TIDMS | 373233 | KGLQP |
| 260950 | RCGCS | 314011 | KNFPV | 351361 | LRLCT | 373239 | LEDTE |
| 260952 | KETKL | 314018 | DQSTV | 351363 | SEWII | 373244 | QNNLT |
| 260953 | IRFSF | 314029 | CKLTV | 351375 | LLEEL | 373249 | PHKQQ |
| 260956 | GAGDQ | 314030 | QCAHQ | 351379 | RMQGA | 373253 | PCIAL |
| 260967 | FSKCW | 314032 | GQTNG | 351380 | MTAVA | 373254 | DRRKA |
| 260968 | FIPLK | 314036 | QKLND | 351384 | QVFES | 373257 | QAPPP |
| 260970 | EDKSG | 314040 | NNHLL | 351390 | MFEKD | 373261 | AELNF |
| 260983 | TFGLE | 314042 | SRLGP | 351391 | SIMHI | 373265 | ISLQD |
| 260985 | AQAKL | 314048 | QHTNV | 351392 | NITII | 373272 | RILVF |
| 260988 | VMDLY | 314067 | MKSNR | 351395 | KSWWK | 373273 | AGYWD |
| 261006 | KQDYH | 314073 | RRIQQ | 351401 | ADDPV | 373274 | TGEKP |
| 261007 | LNQQG | 314077 | SQLLS | 351409 | DSKPY | 373278 | LMSEF |
| 261015 | SHVGA | 314080 | RFSPT | 351410 | VSLSV | 373282 | HATRN |
| 261024 | NTSVV | 314093 | FVRNP | 351411 | EEERE | 373283 | FKVHP |
| 261034 | DEDTF | 314099 | SQATP | 351416 | MNQLG | 373287 | DSETY |
| 261037 | LLYPM | 314103 | GEPGR | 351424 | CVLVA | 373288 | LYERD |
| 261038 | VVVSV | 314105 | CLYLA | 351425 | ASFSF | 373290 | TSVGF |
| 261047 | KVKMK | 314116 | KKKPF | 351430 | SYCIR | 373298 | CRYFT |
| 261058 | EVTRL | 314124 | AARTS | 351432 | CFMPN | 373300 | LDPKS |
| 261070 | LGFKI | 314126 | GGRRR | 351446 | WKSDC | 373301 | MVQIG |
| 261167 | MEGLL | 314132 | LLPER | 351455 | EKNIG | 373304 | YKQVT |
| 261168 | STQSS | 314151 | IVANP | 351473 | IFHYI | 373312 | QQNEQ |
| 261169 | APQVR | 314153 | IPESY | 351475 | RLLTS | 373319 | GDLSA |
| 261170 | ESTYF | 314156 | GEANN | 351480 | GAGVS | 373331 | GLRLQ |
| 261172 | VGSLV | 314163 | GHCAR | 351482 | RHGEV | 373332 | LVSCT |
| 261173 | LETSL | 314171 | RELMA | 351483 | SNYRT | 373340 | TSDSD |
| 261177 | QRHFY | 314173 | NSIQI | 351486 | LDVLG | 373343 | APEQP |
| 261180 | KALRH | 314193 | WIAAL | 351490 | RMEAS | 373345 | RELQS |
| 261182 | ECKQQ | 314196 | KHKCN | 351492 | ELYYQ | 373347 | KSGTM |
| 261183 | NFMFK | 314197 | LSGGL | 351503 | VTLVA | 373354 | GSYFD |
| 261187 | RETNI | 314223 | AGQHW | 351506 | PPKDR | 373360 | SRLPR |
| 261191 | KASRQ | 314228 | LPFFR | 351509 | NIGSL | 373397 | EKKAL |
| 261192 | TIVLS | 314231 | LLRNM | 351519 | WEWGK | 373399 | EAHTE |
| 261195 | GEYKN | 314299 | TCAKR | 351520 | APGTL | 373404 | ASAQS |
| 261196 | NAAAN | 314309 | IDMPD | 351524 | YSYFK | 373411 | SNSEN |
| 261197 | LQPTS | 314311 | FSEYD | 351530 | SSFLV | 373413 | NLCLT |
| 261200 | VRADM | 314324 | ASDAN | 351536 | HHPAV | 373414 | YEKNI |
| 261203 | QNHMS | 314331 | QVRIE | 351539 | QELWK | 373420 | SSNLP |
| 261205 | LAVKK | 314343 | PGGTL | 351541 | LETAQ | 373426 | CLVAR |
| 261206 | MLKQA | 314348 | LRSQS | 351543 | AVATT | 373427 | LRARK |
| 261207 | SKLSK | 314365 | LTWLL | 351552 | SATWK | 373433 | ARCYS |
| 261208 | ESEDL | 314380 | GRAKE | 351559 | YANFK | 373441 | LSKVE |
| 261211 | QSMLF | 314396 | ITVKQ | 351568 | GLSRT | 373454 | ESGGF |
| 261214 | RHMLV | 314398 | VEPLG | 351569 | PCFQR | 373463 | VETSL |
| 261226 | MIIPR | 314400 | GDGDH | 351575 | VDETE | 373476 | LQRQY |
| 261233 | QYKKE | 314401 | RVHEN | 351589 | KAKGK | 373477 | GVKRK |
| 261234 | MNNKS | 314407 | RAAFQ | 351591 | STTRV | 373479 | EUPSN |
| 261244 | GADTF | 314414 | SILKN | 351593 | RGKNV | 373480 | NLKRL |
| 261245 | FWQPS | 314418 | LLYSH | 351595 | LPKCL | 373481 | GPSCA |
| 261247 | GANGH | 314420 | GNCTS | 351596 | RLSSQ | 373484 | LCHLH |
| 261249 | IVSNT | 314440 | SLPRV | 351598 | HSEIL | 373485 | PEPAS |
| 261250 | LTSKH | 314444 | HSPVG | 351602 | SWFER | 373487 | KVLRH |
| 261254 | RDIDL | 314458 | KCCIF | 351605 | CHSDT | 373490 | TYLNN |
| 261263 | CCSSG | 314463 | ESRRP | 351608 | LGVYK | 373509 | KKYLS |
| 261266 | HRGPV | 314477 | LVSCL | 351618 | MTAAV | 373518 | FQENF |
| 261267 | QGCGV | 314480 | ELDMD | 351625 | HATVI | 373522 | KIAAF |
| 261275 | VLDQV | 314487 | SATHW | 351627 | LVPTS | 373534 | PLLQP |
| 261292 | TVELP | 314488 | QRFLI | 351631 | ILTSP | 373536 | LSSAK |
| 261296 | EPFPV | 314491 | DVDFF | 351632 | PPKPF | 373538 | PNYFI |
| 261302 | ETTKN | 314498 | CMKSL | 351642 | AAHTV | 373539 | PLRTL |
| 261303 | EGLYL | 314499 | SRPLF | 351644 | FCVGK | 373555 | EMGKR |
| 261304 | VEATR | 314500 | DMVSS | 351646 | LIAIR | 373562 | RISLR |
| 261308 | NITVK | 314501 | TSAKE | 351650 | MSGIR | 373565 | FKSKK |
| 261312 | GFHLN | 314505 | MRKKS | 351653 | KNPTK | 373566 | KMLLK |
| 261313 | QLSGK | 314508 | LWRRQ | 351657 | VNYLV | 373567 | GAHSV |
| 261318 | ELFPA | 314510 | NWLVW | 351659 | MDSTL | 373570 | NQSVV |
| 261326 | QDVTS | 314518 | AQQET | 351664 | TGAQG | 373572 | EGTEV |
| 261332 | NVVKV | 314520 | MLTDV | 351669 | QLPVS | 373575 | KTTLD |
| 261336 | EHGNV | 314528 | LPREG | 351671 | KVKNM | 373583 | ISPLH |
| 261340 | VCLFE | 314543 | FSHAK | 351673 | PLLDR | 373586 | GGEES |
| 261349 | CTDSS | 314544 | IFFLD | 351674 | LSLKT | 373588 | YGSGH |
| 261353 | PSDNS | 314556 | QGLVC | 351682 | MAQLH | 373592 | PDLGF |
| 261359 | SDFQL | 314560 | RILNV | 351686 | HITKP | 373594 | RVPEA |
| 261364 | SIEVD | 314566 | KQEQP | 351689 | FYKCE | 373596 | TQDSK |
| 261366 | SCAIM | 314575 | RRLSP | 351695 | TLTPC | 373601 | LQQIK |
| 261367 | KELFL | 314606 | LHNEL | 351697 | LLDQF | 373602 | FVDTC |
| 261374 | DFRWE | 314608 | SQIEC | 351703 | TDEDD | 373622 | SPKTN |
| 261377 | PGLCS | 314615 | DNQNW | 351706 | ISGVP | 373627 | KHRKK |
| 261381 | DGRLR | 314620 | NDSSP | 351707 | ILSTT | 373640 | SGISM |
| 261383 | CQLDN | 314624 | QSGGR | 351716 | KLEKR | 373648 | SNKPI |
| 261386 | CEPHF | 314625 | SWQQW | 351723 | KMKPK | 373657 | SEAES |
| 261396 | VQGQI | 314649 | CYGGL | 351727 | QDFNC | 373658 | VKAYS |
| 261401 | AKIAA | 314667 | RETAS | 351731 | YIGWC | 373669 | EALCK |
| 261402 | CSKLN | 314694 | GWSCR | 351732 | QRPPH | 373682 | QRFLD |
| 261405 | RKCSK | 314709 | EAGQA | 351737 | TIHEE | 373684 | KAGKL |
| 261406 | VWGVI | 314724 | EDSNV | 351740 | LKAQP | 373692 | IKKRL |
| 261407 | LKKME | 314754 | RQRSK | 351741 | SRKKI | 373694 | STAHG |
| 261413 | AKASA | 314768 | NKVAA | 351755 | DHSVV | 373696 | SFHLS |
| 261416 | NHENM | 314774 | WRPWP | 351760 | RQRSK | 373700 | NQPGP |
| 261427 | LLLSN | 314775 | MYTCS | 351766 | LSPRP | 373702 | YQLLT |
| 261434 | KTKDL | 314776 | SRTSP | 351769 | PLEGW | 373712 | LHPRG |
| 261435 | KVMLK | 314783 | TMKRN | 351777 | DDLYG | 373713 | KEKID |
| 261438 | RHMLV | 314806 | FLFDL | 351781 | EKAAC | 373715 | KILDL |
| 261439 | EPTGD | 314807 | DFYIL | 351783 | LPVYR | 373718 | GPNCA |
| 261441 | STTSV | 314810 | EPIGN | 351787 | VLLPK | 373722 | ELKSA |
| 261448 | DDDDE | 314813 | GEEEE | 351789 | QGPLD | 373726 | IHIAY |
| 261455 | TSHIS | 314827 | PGVLT | 351793 | FYCQN | 373752 | FFFPF |
| 261461 | NTSAE | 314829 | FHISL | 351807 | GRKRK | 373765 | NISSC |
| 261464 | DLEDL | 314831 | QEPLD | 351811 | TPSLP | 373767 | LRRYP |
| 261465 | RFINK | 314835 | ASYLL | 351813 | EQEEA | 373770 | LDMMS |
| 261475 | DLDDT | 314837 | SSLVG | 351834 | STIKP | 373773 | MRMPL |
| 261479 | LAERD | 314846 | SLCLF | 351857 | QDASS | 373783 | QLSPQ |
| 261483 | RIQLR | 314853 | RQVRG | 351868 | PPRCL | 373785 | YLVSC |
| 261486 | LKTET | 314854 | YCFDI | 351881 | RIQAI | 373786 | PIFHH |
| 261488 | GIKTT | 314897 | RPADF | 351883 | ETTSQ | 373808 | KAKLT |
| 261497 | FLEYE | 314901 | SYYMW | 351893 | FQIFI | 373827 | EVQRK |
| 261499 | QSFPQ | 314910 | GGEEI | 351894 | RRKSK | 373828 | QPGAG |
| 261503 | KYFET | 314914 | AEPHL | 351896 | FGPGS | 373846 | RETFF |
| 261507 | EKKTE | 314929 | QARQN | 351905 | LNTTK | 373848 | LCRLS |
| 261509 | YISRH | 314946 | VVGPY | 351926 | LAQQF | 373853 | QETQI |
| 261510 | ETLNF | 314949 | SDEEN | 351929 | LNSER | 373859 | TLDFK |
| 261511 | DQLAA | 314951 | LDGKN | 351933 | PSDHS | 373860 | GIHLC |
| 261514 | NSTTL | 314971 | RGLKM | 351937 | KTCLD | 373862 | MTPFV |
| 261517 | PQLPS | 314973 | ITVDD | 351944 | LEKEK | 373863 | RELEP |
| 261520 | KRKIT | 314976 | VQMEN | 351945 | TFLNS | 373892 | FTKQR |
| 261523 | MQIDG | 314978 | VLFEK | 351947 | TSMGL | 373895 | VFQIP |
| 261530 | SPGYS | 314992 | ASGRI | 351956 | TTVTP | 373899 | SYQNT |
| 261531 | KRRKE | 315006 | NTVCA | 351957 | QKPDQ | 373909 | LEPGD |
| 261534 | DSWDF | 315008 | GAQSI | 351966 | LLLSL | 373913 | EHDGT |
| 261535 | SGKTR | 315011 | KDSMN | 351967 | CCVIS | 373933 | AAEAP |
| 261537 | RILLY | 315013 | PRAKL | 351975 | PYLSR | 373934 | SHECC |
| 261556 | NRKNV | 315017 | EKEDA | 351976 | FPAAP | 373937 | TPPNL |
| 261558 | GSLLL | 315021 | FTLVA | 351978 | VFQQQ | 373941 | GPCQC |
| 261560 | ETLQM | 315035 | GTYLY | 351981 | NEKST | 373948 | KSIPT |
| 261574 | LLNSA | 315047 | SSVKF | 351989 | VVCNT | 373952 | EPHLF |
| 261584 | VYHYS | 315051 | SLCLF | 351990 | ATFQL | 373963 | YSQRY |
| 261588 | RRRRC | 315056 | ISVST | 351997 | LILGD | 373964 | LRRFE |
| 261590 | QHSYS | 315060 | AALSL | 352005 | RVLTN | 373980 | GINVV |
| 261593 | FQVNI | 315064 | GIHLW | 352007 | TSSSV | 373986 | IPPTV |
| 261596 | LDDLS | 315070 | KSEWV | 352011 | ADLDP | 373996 | PSMRN |
| 261597 | KSSEE | 315081 | AQAPS | 352015 | AVSSA | 373998 | LFRRT |
| 261598 | SYSGG | 315098 | EPISN | 352021 | RHSGE | 374001 | FSNTF |
| 261600 | NETNS | 315106 | VTPQN | 352026 | MTLLF | 374005 | FQVTS |
| 261609 | YLTGH | 315111 | PEEAE | 352028 | VFPSK | 374011 | PGTGP |
| 261615 | CLSLL | 315112 | PRRRK | 352040 | APQRL | 374013 | SRPPQ |
| 261622 | VPQET | 315118 | WVDPS | 352042 | FTQSR | 374014 | HKKQH |
| 261623 | TDEVV | 315130 | KHREE | 352043 | HERSG | 374016 | KESHS |
| 261636 | LKSRQ | 315136 | QPVYL | 352044 | LQNIH | 374027 | QYLGI |
| 261637 | LAMVE | 315137 | FLGLP | 352047 | LDSRC | 374034 | RPLGF |
| 261643 | GPPPS | 315139 | TMETE | 352053 | MRMPL | 374035 | YDDIN |
| 261647 | NSVKL | 315147 | PMPTT | 352063 | AGRPV | 374036 | HVSSD |
| 261650 | GCWHQ | 315160 | QTCYP | 352064 | CKHKL | 374037 | GGLAK |
| 261651 | SLLQF | 315167 | NSISI | 352065 | AKISK | 374045 | CQLSE |
| 261652 | GGPGA | 315177 | DQPGK | 352071 | ENGNL | 374049 | KVTLS |
| 261654 | DLSAV | 315182 | QTPNN | 352084 | KEDYR | 374050 | LSESC |
| 261655 | VHFTP | 315192 | FHLGL | 352097 | STISN | 374051 | VQESV |
| 261657 | GTAED | 315194 | ERGSA | 352101 | VGSDP | 374069 | KALPQ |
| 261658 | ETQVL | 315202 | NYLNK | 352111 | PRRRR | 374089 | TEPSD |
| 261659 | SPKCD | 315203 | VPRKA | 352117 | KLLEA | 374092 | LHENV |
| 261660 | LIKKK | 315205 | NYQYR | 352119 | KAKGK | 374097 | SSESS |
| 261667 | KEFNF | 315212 | HPIYI | 352121 | EKHSA | 374110 | RDGCY |
| 261673 | EVEAR | 315214 | CRVAC | 352132 | PEFQL | 374113 | NGASA |
| 261674 | QTSAS | 315224 | ATASN | 352137 | VFGKS | 374118 | WTAWV |
| 261681 | STWLR | 315232 | CPASR | 352143 | LSLPH | 374121 | MEAVM |
| 261683 | TILVE | 315236 | RVRQS | 352144 | FWFGA | 374125 | TMCSP |
| 261692 | RNARS | 315247 | YTIEI | 352148 | LEDNK | 374136 | RETVA |
| 261693 | QEAKL | 315263 | LNYKV | 352156 | YTSSK | 374139 | DKGYS |
| 261700 | GKVGR | 315265 | PTDHL | 352157 | LGCCR | 374145 | LPSPD |
| 261708 | QTGHL | 315289 | SFCFK | 352162 | KQHKA | 374147 | IKQEP |
| 261712 | AKKLT | 315295 | THLHG | 352163 | LSLRL | 374152 | EMTDV |
| 261713 | EQSAI | 315299 | VLVSK | 352167 | QPSSL | 374159 | PGPPK |
| 261716 | HMSYT | 315303 | PPPQQ | 352172 | EDRPV | 374171 | VESNV |
| 261721 | IIFYT | 315309 | EPMEH | 352173 | MIGSM | 374175 | ALAST |
| 261722 | QALTQ | 315334 | TIWIQ | 352175 | TVLEK | 374181 | FSSFV |
| 261723 | NRDSS | 315343 | SSCPL | 352176 | LIWIF | 374184 | AQTRK |
| 261726 | LEWEF | 315348 | TLNWP | 352177 | PVTPS | 374185 | SGIPK |
| 261729 | GPLGP | 315351 | LPSQA | 352182 | WRWQC | 374187 | QTKLK |
| 261731 | EAAVW | 315357 | AWCLM | 352185 | GKLLL | 374195 | STHSI |
| 261733 | PQKNS | 315370 | KHSSK | 352193 | SSGGS | 374205 | FYSHS |
| 261735 | EKEEL | 315371 | AKSTL | 352215 | APATT | 374218 | GQPRS |
| 261739 | SLTDK | 315373 | YECGQ | 352216 | ISAAS | 374219 | ILQKA |
| 261740 | DDAPL | 315379 | LESTD | 352219 | ESLRR | 374221 | TSHSA |
| 261741 | QTLQL | 315383 | QELSH | 352221 | PPKSK | 374226 | AGASL |
| 261745 | KKLKI | 315386 | SREEL | 352222 | ELEIS | 374234 | ATGLF |
| 261749 | KIQKI | 315387 | GGGAH | 352232 | QYSFV | 374235 | TPLNG |
| 261751 | AAQRD | 315397 | NIQFN | 352233 | SSDLR | 374237 | LNYIC |
| 261752 | PKLPT | 315398 | LSPSF | 352239 | NSAAQ | 374239 | ALVQM |
| 261755 | ALLPS | 315410 | HPVLW | 352241 | LGHEV | 374240 | AALGL |
| 261758 | KREDL | 315417 | ISWAM | 352248 | DAEDY | 374245 | EALKN |
| 261772 | GDVKN | 315465 | SYTLV | 352249 | HSCVH | 374250 | TIGTR |
| 261776 | RRVVL | 315474 | KFYSS | 352252 | RGERA | 374252 | FTGTW |
| 261778 | IMVLP | 315476 | ILLGD | 352254 | CLSPR | 374259 | GSLRV |
| 261783 | ARVRI | 315482 | AAALW | 352257 | KHFQD | 374260 | STVKT |
| 261789 | NLKMD | 315490 | GPPWP | 352259 | SHCLC | 374265 | LKVAI |
| 261796 | CTCIF | 315497 | QKFRL | 352262 | RKFLN | 374268 | PNQMH |
| 261797 | LQNTR | 315505 | RTHSG | 352265 | VKPVG | 374274 | LRGDV |
| 261798 | NMKGF | 315512 | GAAIF | 352268 | FVKGL | 374280 | RRGLI |
| 261799 | EDSFL | 315554 | SLTGR | 352269 | NKDVA | 374283 | KDYRS |
| 261800 | EEVMF | 315557 | DPNYD | 352271 | GVEFC | 374290 | KLKKA |
| 261804 | AAVAE | 315560 | FSQGQ | 352272 | ETEEL | 374292 | YNIMD |
| 261811 | WDMLT | 315564 | THVQT | 352273 | RPRRV | 374303 | LIRNR |
| 261812 | KRPKY | 315568 | PDEKP | 352275 | TSHCT | 374304 | KAAPQ |
| 261813 | ARRAQ | 315569 | DSSTS | 352282 | WISSN | 374306 | WPGIV |
| 261817 | IPLQR | 315571 | ATQFP | 352284 | NWAPP | 374309 | PGTES |
| 261819 | LINHL | 315602 | AREDA | 352288 | KVTDL | 374315 | SSYPQ |
| 261822 | NSEEM | 315615 | NNLLQ | 352294 | WSLSW | 374323 | KGFHV |
| 261826 | FKSPY | 315627 | GTKAL | 352296 | AGRPV | 374328 | HEQCK |
| 261835 | PPPPC | 315630 | KKTTE | 352303 | CIPLG | 374332 | ACVEK |
| 261837 | LRVWA | 315633 | ARELI | 352308 | SRWDS | 374347 | KNIGL |
| 261839 | FLNRL | 315635 | DEIWL | 352314 | VPYNK | 374359 | LQDLQ |
| 261842 | VMEGS | 315637 | PPFSE | 352315 | ECPAS | 374373 | FPKKK |
| 261844 | FSPRC | 315644 | MEMAV | 352319 | PNEKR | 374374 | LSEVA |
| 261845 | LKHLN | 315651 | SRASC | 352320 | LVVGL | 374375 | TPHPP |
| 261854 | QIVQQ | 315654 | VRAGR | 352336 | SKFYS | 374376 | FEFQV |
| 261858 | RAKHN | 315662 | SRSLV | 352358 | WEYQI | 374377 | GPATL |
| 261862 | FLGSR | 315664 | KKPFQ | 352373 | ITCYI | 374387 | RKKIS |
| 261866 | DMLAG | 315669 | PGDAC | 352377 | GDTSL | 374390 | ESKGS |
| 261867 | QSSSP | 315674 | KKFKK | 352385 | KYEIL | 374408 | PQGMS |
| 261868 | YEDFM | 315680 | LTIPA | 352386 | HILTA | 374409 | PDSWV |
| 261875 | GFLPV | 315682 | KKVTP | 352395 | RWLSD | 374443 | SYAAG |
| 261879 | NQRSR | 315684 | FVWTE | 352401 | RSLDR | 374447 | NALSA |
| 261880 | SDEEH | 315687 | HRISA | 352402 | LHRYH | 374455 | HPPPL |
| 261881 | ITEAR | 315700 | ESLDP | 352412 | SIVSS | 374461 | NQERC |
| 261883 | QPLIN | 315702 | GESEA | 352413 | ICSDV | 374471 | DHGEN |
| 261884 | QNKAV | 315706 | RIKVM | 352414 | PQPLT | 374476 | GRGKH |
| 261887 | SDTDY | 315713 | WMSKS | 352425 | VDWLE | 374481 | LKETI |
| 261888 | NRAKR | 315731 | EVGTD | 352431 | GLTPS | 374483 | IPAHH |
| 261890 | CCQNI | 315743 | LCFPS | 352438 | GMGSS | 374490 | LLLQV |
| 261891 | RSSTS | 315757 | GMKRV | 352440 | YPFQI | 374497 | WKKQN |
| 261892 | CPVSV | 315759 | VAQSF | 352442 | YTSSK | 374501 | NYALN |
| 261893 | KDRSD | 315762 | KVHQD | 352443 | PQPPE | 374507 | EYGCV |
| 261900 | PPLPK | 315768 | MPSDF | 352444 | EKPYE | 374523 | YVVFL |
| 261908 | AITTA | 315774 | DYLYR | 352447 | GLLKE | 374529 | AAWMR |
| 261917 | LPSNL | 315775 | KRLRL | 352455 | RPNGL | 374547 | FCGSS |
| 261918 | GLLVH | 315791 | QKRIR | 352456 | CKRNN | 374552 | PRHRF |
| 261921 | KIVQS | 315794 | TWRIS | 352458 | VLFEK | 374557 | KKKKK |
| 261937 | TDNSY | 315835 | WHLEG | 352461 | WSYSL | 374565 | VPPQW |
| 261942 | DLTDE | 315849 | PGHVD | 352463 | QSERG | 374581 | MERKL |
| 261944 | DTTDL | 315859 | SNSSR | 352469 | KKKVK | 374586 | SSAGK |
| 261947 | EVEWF | 315870 | KGERD | 352470 | STRAS | 374587 | PETEP |
| 261948 | TTLED | 315872 | CSLWR | 352479 | TSITY | 374592 | PLGKP |
| 261951 | LPGRR | 315878 | ELLTS | 352487 | APCQS | 374611 | KTSFL |
| 261965 | RSSHT | 315906 | PYDGV | 352497 | DSRHW | 374614 | NSDVC |
| 261973 | IGLVK | 315914 | SSELT | 352498 | DDWES | 374620 | CAVPR |
| 261978 | CTAKE | 315915 | GAKQP | 352510 | EWEVI | 374630 | FGKKK |
| 261980 | LEDMA | 315931 | NYYRY | 352514 | VWRPY | 374632 | NQSAP |
| 261991 | SDSVA | 315932 | SGLLC | 352516 | EAAKD | 374642 | GRAKA |
| 261994 | NPTLL | 315945 | SEATK | 352522 | AAARS | 374643 | PCYYA |
| 262017 | TYRGT | 315949 | EGLTN | 352527 | SPKGT | 374777 | NRGEC |
| 262018 | ILDQH | 315950 | KILEC | 352536 | LLPTP | 374778 | YSTPP |
| 262027 | GKKKK | 315955 | IMNSS | 352540 | LRKRK | 374779 | DNFPL |
| 262030 | EEHSS | 315988 | VLKVN | 352544 | DMFKK | 374780 | NSYSP |
| 262031 | FQFNK | 316001 | FFVCC | 352545 | RKIPP | 374781 | YNYPP |
| 262032 | EHKVG | 316007 | KISWK | 352555 | DCECY | 374782 | YNLPP |
| 262033 | MPDLE | 316014 | WRCHF | 352565 | LELKQ | 374783 | YSYPP |
| 262041 | EHAHL | 316021 | SAVTS | 352572 | PMIKQ | 374784 | NSYPP |
| 262042 | GDADM | 316029 | LRDEH | 352575 | RTAMQ | 374785 | SNWPP |
| 262043 | TKSKR | 316030 | RTGGN | 352584 | FNAEY | 374786 | NSFPP |
| 262053 | SNKSV | 316032 | AVGSL | 352585 | LESFW | 374787 | NNWPP |
| 262055 | LGTRR | 316042 | GGSSF | 352592 | AHFRD | 374788 | NSYPP |
| 262056 | EDYAE | 316053 | WMDSK | 352599 | VPKSN | 374789 | NSYPP |
| 262059 | PFTFE | 316054 | FVDVM | 352601 | EPLLT | 374790 | GSSPP |
| 262061 | WSLLR | 316092 | GSPEL | 352603 | YVIRI | 374791 | SSSLP |
| 262065 | FMRHL | 316093 | KGTVA | 352604 | MNQEK | 374792 | ATQFP |
| 262067 | PSAFL | 316098 | REGSA | 352606 | CYRQH | 374793 | NSAPP |
| 262069 | EEPAL | 316101 | LPQAR | 352608 | EDQLS | 374794 | LQTPP |
| 262074 | GTTAD | 316109 | KGTLE | 352612 | KTHKA | 374795 | THWPP |
| 262077 | VRRRK | 316114 | EPKKK | 352614 | RERFG | 374796 | DNLPP |
| 262085 | PPERD | 316121 | IRTHK | 352622 | DTNGL | 374797 | YNAPP |
| 262089 | QSVLC | 316122 | VPTWL | 352626 | SSKIF | 374798 | YSTPP |
| 262094 | KKCIC | 316129 | RRGRR | 352627 | KAKGK | 374799 | IEFPS |
| 262095 | TCTKA | 316136 | DDTKM | 352645 | AISGC | 374800 | DNLPP |
| 262096 | MENET | 316137 | EIHPG | 352655 | FDIQF | 374801 | THWPP |
| 262101 | VTCTL | 316149 | GERFC | 352656 | VSLTA | 374802 | IQLPP |
| 262102 | SISPR | 316152 | PLYYI | 352657 | NVQTV | 374803 | AQDPP |
| 262103 | EFNDM | 316173 | RAAFI | 352659 | LELLS | 374804 | ATQFP |
| 262105 | TVRLL | 316176 | AMNNI | 352668 | KLVLG | 374805 | GSSPP |
| 262109 | EKSSD | 316180 | RWGKN | 352671 | SDPGV | 374806 | GNKHP |
| 262113 | SAAGQ | 316193 | KSKRS | 352672 | PHFKH | 374807 | NSYPP |
| 262120 | MNCMF | 316202 | TTPSP | 352673 | LQSRN | 374808 | NSYPP |
| 262126 | ELTPI | 316203 | AGTPK | 352675 | GKAKH | 374809 | NNWPP |
| 262127 | YRSVL | 316222 | ECETI | 352676 | DDEDE | 374810 | FNNYP |
| 262133 | DRGSH | 316224 | KLSAT | 352678 | EPDSD | 374811 | NSFPP |
| 262134 | DKKDD | 316240 | RPFQR | 352682 | PRGPV | 374812 | SNWHP |
| 262138 | RTTPV | 316244 | KFCRQ | 352683 | SAASS | 374813 | FSYPP |
| 262139 | KTKQS | 316255 | RSSST | 352685 | DLTGL | 374814 | YSTPP |
| 262146 | NVSDS | 316262 | AARSP | 352702 | KETHL | 374815 | YSFPP |
| 262150 | VQSNN | 316264 | LSDED | 352704 | ILQQN | 374816 | YNLPP |
| 262158 | IFNSR | 316284 | LRQMC | 352706 | SDLKF | 374817 | WGTGI |
| 262160 | CSSMS | 316302 | QRVVY | 352707 | STLEG | 374818 | GSGIS |
| 262161 | IISLD | 316310 | RVSHA | 352708 | KHLLS | 374819 | WDSNT |
| 262173 | FEKQQ | 316329 | GDSSA | 352709 | ELKDQ | 374820 | YDSSN |
| 262177 | TKGNH | 316338 | TLAGR | 352710 | IMYIA | 374821 | ESSAN |
| 262178 | ETSVI | 316357 | QTKDQ | 352711 | PLQVV | 374822 | SSLSA |
| 262186 | SDPGS | 316377 | AQTRL | 352712 | TTVMY | 374824 | HSNSK |
| 262187 | SCSVM | 316385 | FDIHL | 352716 | EFRSH | 374825 | SLSAG |
| 262188 | VVRNT | 316390 | AGQLI | 352717 | AFLKH | 374826 | NSLNA |
| 262189 | RKWMN | 316397 | KKSGG | 352719 | MELTV | 374827 | GSNFV |
| 262193 | SLRKD | 316400 | LWRRQ | 352722 | VAGSD | 374828 | HSSAS |
| 262198 | FEYEP | 316417 | QFLCA | 352724 | SSNII | 374829 | SLSGP |
| 262207 | VFAVV | 316423 | FFFFF | 352731 | GQPMI | 374830 | YSGAR |
| 262209 | HHLEP | 316426 | NQEKG | 352734 | YLLPL | 374831 | HSSAS |
| 262211 | EDLFL | 316431 | NIVFN | 352738 | TGQRR | 374832 | SLNGP |
| 262213 | KEKSS | 316454 | GSKAT | 352739 | HMPQL | 374833 | YGGAQ |
| 262215 | GINKQ | 316457 | ARGTP | 352752 | APGAH | 374834 | SLSGS |
| 262219 | VALLH | 316465 | CGRPS | 352757 | VGPLI | 374835 | PSNAS |
| 262225 | VRRVV | 316476 | EMVLE | 352770 | AFNLS | 374836 | SLNGP |
| 262227 | LDIST | 316484 | KGQLS | 352780 | PTNTL | 374837 | SSYTF |
| 262236 | CGWAR | 316490 | FSAMS | 352782 | FLIKD | 374838 | HATQL |
| 262238 | AKNNQ | 316494 | ADEGC | 352785 | HYTQQ | 374839 | ADNNL |
| 262241 | YASAS | 316496 | CFSYG | 352790 | TSHCT | 374840 | SGTYP |
| 262244 | SRMCH | 316510 | ALEHF | 352798 | MTASK | 374841 | GSSTF |
| 262259 | GFTKQ | 316512 | NSSSN | 352799 | RRTRC | 374842 | DEDNP |
| 262262 | EVRTQ | 316518 | QLLKD | 352802 | KQAAE | 374843 | DHPTV |
| 262265 | LPVPS | 316527 | PPPRR | 352814 | QLTIF | 374844 | SGNHL |
| 262283 | PLHGR | 316532 | IKEIA | 352819 | GERDL | 374845 | SSSTF |
| 262288 | VTQQE | 316534 | SRGNR | 352820 | ETHQK | 374846 | SGTYP |
| 262290 | ASVKN | 316547 | DLFDA | 352821 | KLSSV | 374847 | SSSTL |
| 262291 | EEKTK | 316565 | QTKTP | 352823 | FKSNV | 374848 | SSDHP |
| 262293 | FDEQN | 316566 | PGEDA | 352833 | PTWNI | 374849 | GSYTF |
| 262294 | ENSGR | 316578 | KKQKL | 352834 | RLNSR | 374850 | SSGNH |
| 262300 | TLDPT | 316589 | DDYLH | 352835 | LGFQG | 374851 | SSTAH |
| 262301 | LPGPL | 316590 | LPILH | 352836 | QNWNV | 374852 | GSNNF |
| 262302 | PACLP | 316596 | VQFFM | 352837 | SSGVT | 374853 | DGQVG |
| 262304 | HPSST | 316598 | KLFPG | 352838 | MEEGR | 374854 | DSSTA |
| 262305 | ILEVK | 316605 | EKAKQ | 352841 | IMWEQ | 374855 | PTECS |
| 262306 | PRWGL | 316609 | LDMND | 352845 | RAHLS | 374856 | PTECS |
| 262313 | QALLP | 316613 | RGWAG | 352846 | IGKFD | 374857 | PTECS |
| 262316 | DAQLH | 316628 | VISCG | 352849 | SKGQE | 374858 | PAECS |
| 262319 | PPASP | 316632 | VLLEA | 352872 | TEGKK | 374859 | PAECS |
| 262320 | VEKVD | 316638 | LQWGI | 352876 | VETNG | 374860 | NGEKS |
| 262340 | LFKKS | 316640 | EDFAA | 352882 | PDLFI | 374861 | NGEKS |
| 262345 | DSLML | 316644 | SRGLS | 352900 | PLQIT | 374863 | CWIRH |
| 262352 | QTSQF | 316647 | ASSNT | 352904 | KKFSS | 374864 | CAAWD |
| 262354 | HYHEN | 316649 | CPDIY | 352916 | RRMQQ | 374865 | ALWEV |
| 262365 | LNLTL | 316651 | FLQGF | 352918 | ARKTV | 374866 | ATWDR |
| 262366 | PAVVN | 316661 | SSTVN | 352920 | QMKEI | 374867 | ATWDR |
| 262370 | ELTPL | 316664 | RLRKR | 352925 | WHPWL | 374868 | ATWDG |
| 262374 | LVMDT | 316670 | NPKAV | 352926 | RLVKE | 374869 | ATWDR |
| 262375 | KMFTS | 316674 | YRKCY | 352928 | MHEDT | 374870 | ATWDG |
| 262376 | VPRKL | 316675 | PLGVR | 352929 | DHLGK | 374871 | ATWDR |
| 262383 | SQHAQ | 316681 | LFRRS | 352932 | TSPFP | 374872 | ASSEA |
| 262384 | AMVLD | 316710 | NKPVF | 352933 | TMSAH | 374873 | CASSQ |
| 262393 | PLVWE | 316729 | CCSII | 352936 | PPSDE | 374874 | CASSQ |
| 262394 | LSYRI | 316737 | SGGCC | 352941 | KNKGN | 374875 | CASSL |
| 262395 | RKILS | 316738 | SIKEK | 352944 | AGKKA | 374876 | ASSEA |
| 262401 | FPQSP | 316740 | MLQII | 352954 | GGSCL | 374877 | ASSSA |
| 262406 | PWESL | 316746 | SGAVW | 352956 | TPQTM | 374878 | CASSQ |
| 262407 | DEEGE | 316754 | LNSLI | 352962 | RRFPD | 374879 | ASSYS |
| 262414 | LNYPM | 316769 | LIFLF | 352969 | FHLLG | 374880 | CASSQ |
| 262415 | AFRRR | 316772 | QPEFV | 352976 | AVLQK | 374881 | ASSYS |
| 262418 | VAMPV | 316775 | ETPRE | 352979 | RPMSC | 374882 | ASSLA |
| 262419 | RPTHR | 316779 | TERVP | 352989 | KIPQK | 374883 | ASSDS |
| 262424 | FAVRQ | 316782 | VIFPH | 352990 | GSPQP | 374884 | ASSLT |
| 262426 | KPCVM | 316786 | PAVAR | 352991 | AFMLV | 374885 | CARSL |
| 262428 | DAQTE | 316794 | IQACQ | 352992 | HLQQP | 374886 | CASSV |
| 262429 | HPEDV | 316809 | MTADD | 352995 | DVIFF | 374887 | ASSES |
| 262430 | KNSKL | 316840 | TDAGV | 352999 | EMEPN | 374888 | ASSLA |
| 262432 | LSSTS | 316842 | EPLEL | 353007 | EGWEG | 374889 | ASSES |
| 262441 | EKRSQ | 316845 | APFRK | 353010 | RHGDD | 374890 | CASSL |
| 262442 | LLLQI | 316854 | YLGLE | 353013 | GSSDA | 374891 | ASSYS |
| 262444 | ESTAL | 316861 | LNSYT | 353023 | LAKEQ | 374892 | ASSLA |
| 262445 | PMYVD | 316881 | PKDEL | 353025 | PQKQD | 374893 | CASSL |
| 262450 | YVTDI | 316898 | YYPGP | 353028 | SCKNR | 374894 | ASSYS |
| 262455 | DRDEL | 316901 | DSELI | 353030 | DHYAT | 374895 | CASSL |
| 262457 | NKTKP | 316905 | FNKGL | 353035 | SQKAK | 374896 | ASSYS |
| 262460 | EHILS | 316909 | FSTIS | 353036 | AILWL | 374897 | ASSLA |
| 262461 | LTFYS | 316924 | QAAAK | 353042 | KSDGP | 374898 | CASSL |
| 262462 | GEIKL | 316933 | QSSHA | 353044 | CSGKM | 374899 | ASSYS |
| 262464 | QIQLY | 316938 | LKFHR | 353046 | LDDDQ | 374900 | ASSLA |
| 262477 | QLPET | 316940 | ILIFF | 353047 | DTESQ | 374901 | CASSL |
| 262482 | PVILV | 316948 | LLKKK | 353048 | LGLNN | 374902 | ASSYS |
| 262483 | FESVP | 316950 | LDRNA | 353052 | RPRQR | 374903 | ASSLA |
| 262487 | EAREY | 316955 | FKKCP | 353054 | RVHGQ | 374904 | CASSL |
| 262493 | GCGLY | 316959 | FRHCS | 353058 | PCWAS | 374905 | ASSLA |
| 262498 | NKAKQ | 316974 | QPTTL | 353059 | LQTLE | 374906 | ASSLG |
| 262499 | AQGCT | 316983 | TQVLM | 353073 | LCQLS | 374907 | AISES |
| 262502 | TFYCQ | 316990 | ALEGR | 353074 | YTSSK | 374908 | CASSL |
| 262506 | LTAAR | 317007 | FLLQH | 353084 | RERPK | 374909 | ASSLA |
| 262507 | LNQRR | 317027 | LTTTS | 353090 | SVGGS | 374910 | ASSLA |
| 262510 | APWGT | 317028 | PRRRR | 353093 | VCFRP | 374911 | ASGLV |
| 262519 | RGSLN | 317033 | RLCPA | 353094 | VHEEF | 374912 | CASSQ |
| 262525 | PGVSL | 317039 | KRLNK | 353098 | NGNEL | 374913 | CATSR |
| 262536 | FVCSL | 317053 | SGPTA | 353099 | TGFLS | 374914 | YSSGG |
| 262539 | MLDPS | 317059 | CCAPW | 353104 | GTASA | 374915 | ASSPP |
| 262544 | VSSAC | 317075 | VWLHY | 353109 | VNQGT | 374916 | CASSI |
| 262545 | ILNKN | 317088 | DKSHS | 353114 | GPNEG | 374917 | ICSAR |
| 262551 | IGSYF | 317110 | FSSCK | 353120 | PALAP | 374918 | ASSKA |
| 262554 | QAVLL | 317117 | GVGGA | 353128 | AGLWS | 374919 | ASSQS |
| 262563 | CKNFC | 317121 | LEGEL | 353129 | QKYGL | 374920 | ATSDL |
| 262570 | LEKGG | 317123 | DSDSD | 353130 | GVVPL | 374921 | CASSE |
| 262577 | IKPRL | 317125 | IGEKS | 353136 | SGPES | 374922 | ASSLS |
| 262580 | SQEPH | 317128 | CYSEA | 353139 | PRRRR | 374923 | CASSL |
| 262584 | QEKEN | 317141 | VFCRE | 353142 | RPLSP | 374924 | LCSVE |
| 262585 | IWKGE | 317143 | KSPHT | 353143 | ETEKN | 374925 | KRKDF |
| 262593 | YRSEH | 317144 | QKMKT | 353151 | VPSSI | 374927 | KDSRG |
| 262605 | LYSCY | 317145 | RCGCI | 353152 | RSPDT | 374928 | CAWSV |
| 262607 | DVATK | 317156 | ALGKN | 353165 | MAIKS | 374929 | FCAVR |
| 262613 | LFSNL | 317159 | YRPPK | 353168 | KKDRR | 374930 | LCAVR |
| 262622 | FADLY | 317161 | CKLSS | 353199 | DSDSD | 374931 | YCAVE |
| 262623 | QELYY | 317170 | NNKNH | 353203 | SGGCC | 374932 | CAVRD |
| 262624 | EIRVK | 317175 | QPGAP | 353210 | AADVQ | 374933 | CLVGD |
| 262625 | LQTHV | 317176 | SRTIG | 353216 | LTSTE | 374934 | FCAES |
| 262626 | MVTQL | 317177 | SESQT | 353224 | IDNEF | 374935 | LCALD |
| 262629 | RPYYK | 317199 | DMFKN | 353238 | DGAAP | 374936 | FCAVD |
| 262630 | SSSTT | 317214 | DLTEL | 353243 | EQARK | 374937 | CAVNA |
| 262631 | VQVAE | 317224 | KTSTI | 353245 | PTLLE | 374938 | FCALS |
| 262633 | KLGLR | 317232 | VHEEL | 353246 | KAKTT | 374939 | ICVVS |
| 262637 | LHRAA | 317235 | GKMRS | 353249 | VSPRV | 374940 | LCVVN |
| 262640 | TSLFL | 317255 | ARDCP | 353259 | VIRKT | 374941 | FCVVS |
| 262643 | GPEMA | 317257 | QAPQA | 353271 | SFLTF | 374942 | CAVGA |
| 262644 | KTGHK | 317271 | AKSLQ | 353275 | NELYS | 374943 | FCAAS |
| 262648 | SPERY | 317272 | FWETS | 353278 | KEKKN | 374944 | YLCAV |
| 262650 | GFGQE | 317287 | LELEM | 353290 | MACLT | 374945 | AVSDT |
| 262651 | YQQQP | 317289 | TLCHS | 353291 | ACAQP | 374946 | FCAEN |
| 262662 | ATNLQ | 317300 | KQKGD | 353292 | PLGGP | 374947 | AMREG |
| 262709 | VVFVK | 317310 | RMMSR | 353295 | DQVCT | 374948 | FCALS |
| 262710 | RGGRR | 317327 | WISGN | 353313 | STPKR | 374949 | AMSAQ |
| 262713 | PANYI | 317331 | SHIQA | 353314 | RNHII | 374950 | FCAVS |
| 262715 | FLNWE | 317332 | TVLSF | 353318 | DSDLE | 374951 | CALSG |
| 262716 | SSAEK | 317333 | ARDCI | 353319 | THWSK | 374952 | FCATD |
| 262717 | PAPLW | 317334 | GALND | 353321 | RGCST | 374953 | YCALR |
| 262719 | YDTPL | 317337 | KDMAT | 353324 | DYYML | 374954 | ALSEA |
| 262723 | SEYWF | 317357 | HLLKM | 353329 | TQKGC | 374955 | LCAVQ |
| 262726 | RAFLQ | 317376 | KARKL | 353333 | AGEKS | 374956 | LCAVR |
| 262735 | YRDMY | 317379 | LDGLL | 353343 | EMKIR | 374957 | FCAVE |
| 262738 | DSEKP | 317382 | TSGLK | 353344 | PDTED | 374958 | FCAAS |
| 262741 | PSLCR | 317396 | HIHLY | 353353 | NLYLL | 374959 | ALGEL |
| 262746 | FSKQK | 317400 | HSNHH | 353355 | QSAKE | 374960 | YLCAF |
| 262752 | TSTGL | 317402 | WPHLR | 353373 | LREGQ | 374961 | YFCAG |
| 262764 | IKNFF | 317404 | YLRLL | 353375 | QETSI | 374962 | CIVRV |
| 262765 | NTSER | 317431 | SSDQI | 353377 | HLQNW | 374963 | AVGDR |
| 262768 | DIEDP | 317439 | NHTEL | 353385 | QESSA | 374964 | YLCAG |
| 262776 | SSGVD | 317442 | QDRGQ | 353393 | AQDLY | 374965 | FCAAS |
| 262794 | HQEPS | 317445 | AGEHA | 353401 | IEWEF | 374966 | FCGTE |
| 262795 | QLDGS | 317447 | RFFSK | 353405 | AVATT | 374967 | CILRD |
| 262801 | FNAPK | 317468 | LVAAS | 353408 | EFESM | 374968 | LCGAD |
| 262809 | LQAWP | 317473 | PELVS | 353409 | QLQES | 374969 | FCAGQ |
| 262811 | PTGRD | 317477 | TGLRM | 353410 | SGEKP | 374970 | LCAVE |
| 262812 | YAPSA | 317482 | SSVKF | 353414 | LETSL | 374971 | CAFMK |
| 262817 | DLTKL | 317498 | ATVSP | 353415 | ISLTV | 374972 | CAYRS |
| 262820 | PLSAP | 317501 | RHDTM | 353427 | DTLPF | 374973 | FCAVD |
| 262825 | PGEVC | 317534 | SDAPD | 353428 | DHEMP | 374974 | YCLLG |
| 262839 | VTTRL | 317556 | PFGRK | 353444 | QCCSN | 374975 | ICAVR |
| 262843 | MKTCH | 317561 | KPNIR | 353452 | EEEEE | 374976 | CACDT |
| 262844 | YNHYS | 317564 | SPLAD | 353457 | SVRGA | 374977 | KLFFL |
| 262848 | IFKNF | 317578 | NSNGL | 353458 | KKSRI | 374978 | YYCAF |
| 262850 | CLLMR | 317579 | EKKVL | 353462 | RGTLG | 374979 | RLWSS |
| 262861 | IDKKS | 317580 | FIPYH | 353465 | TSVSS | 374980 | EGPQY |
| 262865 | DVVYK | 317595 | DKKTK | 353470 | EPLPF | 374982 | LQPHA |
| 262866 | SLDDA | 317614 | SSKGK | 353475 | SKEYV | 374984 | IRQGA |
| 262873 | PKHKE | 317617 | SPGPH | 353483 | LGCCR | 374986 | IRQGA |
| 262878 | KDDPM | 317618 | SAKNV | 353491 | ETLPL | 374988 | SLSPG |
| 262879 | KNCSS | 317619 | ISIKE | 353493 | FFLKW | 374989 | DGTCY |
| 262887 | VVPQA | 317636 | CGPQR | 353500 | KKIYI | 374990 | IGQGA |
| 262888 | SQQSK | 317661 | DDDWC | 353508 | AKQGL | 374992 | IGQGA |
| 262890 | LEPAP | 317668 | QSIKN | 353512 | GSPVD | 374998 | FIKVR |
| 262891 | NDLEL | 317671 | IHKNN | 353515 | PSLAS | 375000 | TLFKV |
| 262894 | FLNDT | 317681 | ERQKP | 353516 | ELGKE | 375002 | YYCAR |
| 262901 | ITNGS | 317684 | FCNKI | 353518 | SLSTN | 375003 | YYCAR |
| 262903 | GYTMA | 317686 | EQEGK | 353527 | RSERG | 375004 | YYCAR |
| 262904 | DFMCS | 317691 | EDQTA | 353537 | LYPQY | 375005 | YYCAR |
| 262915 | LSSGI | 317714 | VTVVG | 353538 | IQQDM | 375006 | YCAHR |
| 262916 | STNMD | 317721 | GKAHL | 353540 | SAVAA | 375007 | YYCAR |
| 262919 | PGTCI | 317743 | LTHFS | 353541 | PANNS | 375008 | YCARG |
| 262932 | DREDL | 317749 | AQLQL | 353542 | PPKSK | 375009 | YCAKD |
| 262940 | LELQT | 317752 | RVGKL | 353548 | MKPKF | 375010 | YYCAR |
| 262942 | GLRIM | 317768 | HMFKC | 353556 | NVVNS | 375011 | YYCAR |
| 262946 | FKKDN | 317780 | GLDKV | 353557 | HVRVA | 375012 | YYCTT |
| 262947 | SRTEL | 317786 | DLKKD | 353558 | RWKIC | 375013 | YYCVR |
| 262948 | TRTAV | 317790 | LRSDP | 353564 | TTSVV | 375014 | YYCAR |
| 262949 | ARDHL | 317804 | YEISV | 353575 | QLMEI | 375015 | YHCAR |
| 262953 | YEVVY | 317813 | MPLSL | 353581 | RGERA | 375016 | YYCAR |
| 262958 | EINLL | 317817 | RSSEL | 353582 | CCSEA | 375017 | ECRGE |
| 262961 | GEGLV | 317818 | PLVNL | 353586 | SKKSH | 375018 | YYCAK |
| 262967 | LKQGS | 317823 | SRGAG | 353589 | EEDPA | 375019 | YYCAT |
| 262968 | PATDL | 317835 | THSFH | 353590 | PPQSE | 375020 | YCARI |
| 262970 | RTAAA | 317836 | HEDKQ | 353591 | FEAGL | 375021 | YYCAR |
| 262971 | LVPAC | 317842 | RHTEL | 353608 | ACTKR | 375022 | YYCAR |
| 262982 | SVTLL | 317872 | KSVTV | 353612 | HKFRD | 375023 | YYCAR |
| 262990 | EEQEN | 317891 | REGPQ | 353614 | VSLTA | 375024 | YYCAR |
| 262992 | GKADT | 317892 | ETLKA | 353619 | KKIIV | 375025 | YCARG |
| 262995 | AKSVK | 317895 | QNHQR | 353622 | VFKAP | 375026 | YYCVR |
| 262996 | SAMSG | 317902 | LCWDY | 353624 | YGFGG | 375027 | YCARY |
| 262999 | MDCAT | 317903 | RASSQ | 353630 | CGCLG | 375028 | YYCAR |
| 263012 | TCIHY | 317904 | GEERN | 353634 | RKDKK | 375029 | YCAKD |
| 263025 | VLEAP | 317905 | NMDES | 353639 | SKSSS | 375030 | YYCAR |
| 263026 | AQMEE | 317912 | RALEH | 353646 | DLPCV | 375031 | YYCAR |
| 263032 | PVCAS | 317926 | ERYNG | 353650 | TYEKV | 375032 | YYCAR |
| 263033 | QKLGL | 317931 | DLSKQ | 353654 | CMKNL | 375033 | YYCAR |
| 263035 | AKTFA | 317943 | QFLAT | 353655 | RLRLG | 375034 | YYCTR |
| 263036 | MDCII | 317952 | RVDPY | 353656 | GGEED | 375035 | YYCAR |
| 263043 | YASRR | 317955 | NDLQG | 353657 | AAAEQ | 375036 | YYCAR |
| 263045 | SNSIY | 317958 | EFRRF | 353670 | FPRKA | 375037 | YYCAA |
| 263056 | TLEYG | 317971 | QCRPS | 353679 | KCRVW | 375038 | YYCAR |
| 263062 | AMEVT | 317980 | ALSKL | 353685 | HILFV | 375039 | YYCAR |
| 263071 | RPPEP | 317985 | PNKPS | 353693 | VHPSV | 375040 | YYCAR |
| 263073 | WAQVG | 317992 | LSEDD | 353695 | VGVPI | 375041 | YYCAR |
| 263080 | RCCLH | 317993 | RAHLS | 353698 | FVFPG | 375042 | ARDTV |
| 263083 | APLAP | 317994 | PCSFL | 353699 | PCKVS | 375043 | YCARI |
| 263084 | DQFAE | 317997 | HYENF | 353700 | RPTTT | 375044 | YYCAR |
| 263087 | LEEEN | 317998 | DHCPP | 353701 | PSGQA | 375045 | YYCTR |
| 263088 | LEVWT | 318024 | FSEKN | 353702 | KKAHI | 375046 | YYCAR |
| 263092 | LVAVN | 318029 | PGRCC | 353706 | DTKAG | 375047 | YYCVR |
| 263093 | GTWRL | 318032 | ILGLY | 353708 | YKCRT | 375048 | YYCAR |
| 263094 | AETVL | 318048 | FARLL | 353714 | FLLFL | 375049 | CSCCA |
| 263095 | QVSSL | 318057 | VTTCA | 353720 | EHIEL | 375053 | ACRVN |
| 263097 | EEAGY | 318066 | DPLIG | 353728 | AFNQP | 375058 | ECPAS |
| 263100 | LVAES | 318070 | AATSN | 353729 | STDIF | 375063 | YWWKN |
| 263115 | IDMAK | 318075 | SGLQA | 353731 | CFSLP | 375066 | GPTSP |
| 263116 | SLGCC | 318077 | FPRRD | 353732 | AEMQS | 375067 | PTGFP |
| 263119 | DYMDI | 318085 | EIQMV | 353735 | DCDMS | 375068 | QESPF |
| 263125 | ERLIS | 318089 | ASTDL | 353739 | YFRDK | 375069 | GCWHK |
| 263126 | FSDEY | 318094 | KQGKF | 353741 | DLDDY | 375072 | EAKAI |
| 263150 | LLQEK | 318113 | TNLKH | 353742 | SCSGL | 375073 | FINRE |
| 263154 | PKSPQ | 318115 | RSKQP | 353748 | QITWQ | 375076 | QSLCC |
| 263160 | RVDYS | 318119 | PTQAS | 353759 | CDFTF | 375080 | DLEPL |
| 263168 | EMQNA | 318128 | ERPQL | 353767 | QALTL | 375081 | ETRAQ |
| 263170 | SLFLI | 318131 | ESRKV | 353769 | PLPPL | 375083 | AWTTW |
| 263174 | GKKVI | 318136 | FIFQK | 353770 | LDADF | 375085 | QEVKV |
| 263181 | KGNLR | 318142 | GWFQA | 353778 | KTNEA | 375086 | ANSGL |
| 263182 | VENGN | 318154 | RSESS | 353780 | RDLQE | 375087 | NLGRF |
| 263187 | EKTEE | 318158 | VYKYL | 353783 | RGERA | 375089 | TADCR |
| 263196 | LNTVV | 318165 | DEFDF | 353784 | GEMVG | 375090 | GATPH |
| 263200 | FDGHE | 318171 | LSSFF | 353785 | VQGGK | 375091 | WQMTR |
| 263201 | ISLLS | 318174 | PRHEV | 353786 | EYKKP | 375092 | NQSTP |
| 263205 | CLSAA | 318176 | IAERK | 353787 | HLKIK | 375093 | CQIRC |
| 263207 | VDSWV | 318177 | HSQEQ | 353792 | WSLVG | 375094 | FRESS |
| 263208 | ILRDK | 318182 | WLEEN | 353793 | AFKQE | 375095 | VWAKR |
| 263209 | CTVDV | 318184 | PTDWK | 353794 | DAFPQ | 375099 | LLGSL |
| 263212 | APPRS | 318185 | LLNSY | 353796 | PQTNP | 375105 | RPPRP |
| 263228 | GNEES | 318195 | KTKFE | 353797 | HREDR | 375107 | FSGFY |
| 263233 | FSNQM | 318196 | TDMED | 353806 | KLDAN | 375108 | FSGFY |
| 263238 | FGVMS | 318197 | EGEAY | 353824 | SSESL | 375109 | RSGFY |
| 263239 | RQFSH | 318203 | EKYGI | 353826 | LAVER | 375110 | KSRLQ |
| 263243 | ATTGE | 318208 | WRNRS | 353844 | LKTQD | 375112 | DGQSP |
| 263245 | DRYGS | 318212 | GSWGH | 353846 | PNSPF | 375116 | LLTSQ |
| 263246 | VEAIQ | 318217 | ALNTV | 353847 | FLTWL | 375126 | GVGRG |
| 263253 | TLDIH | 318222 | VQCRK | 353854 | QSHHL | 375127 | PLIPM |
| 263255 | RAHQH | 318226 | YKRIS | 353858 | SVNSL | 375128 | LLEEL |
| 263256 | PNGQS | 318227 | QCMPS | 353863 | GQTFG | 375129 | LLEEL |
| 263257 | PQKVG | 318262 | RLTPA | 353864 | KNSSR | 375130 | CCPGL |
| 263258 | GPLAK | 318264 | DKHIF | 353866 | ELEYL | 375132 | RRPCG |
| 263265 | LQSSF | 318275 | HHQPQ | 353868 | GTWPG | 375133 | SPQSK |
| 263266 | LGGQP | 318279 | SAPQR | 353871 | LESTL | 375134 | AVMQS |
| 263269 | LESEV | 318297 | DKYMK | 353873 | KLTRL | 375135 | GSGRA |
| 263270 | LQSLE | 318304 | THIYI | 353877 | AQEAV | 375136 | AKPKP |
| 263273 | VPQHL | 318310 | SLGLG | 353883 | AGESS | 375137 | TLRTK |
| 263274 | PEDTY | 318318 | ACCLL | 353888 | ARLGR | 375138 | FRDEK |
| 263277 | KGSAE | 318340 | MKAIA | 353893 | VLISD | 375139 | SYSVE |
| 263278 | PDIPS | 318351 | KMINY | 353902 | MVQVI | 375140 | FLPQC |
| 263281 | LESYC | 318352 | KSESS | 353904 | IQRHK | 375141 | AHEQG |
| 263282 | RTLTR | 318355 | LECKL | 353910 | PEAEK | 375142 | YFLNS |
| 263284 | LDMRS | 318373 | HTDSS | 353915 | QPSDT | 375143 | FMTNQ |
| 263285 | YENFT | 318374 | QLSAF | 353920 | FSLTP | 375144 | LCVLS |
| 263301 | FHSTC | 318395 | LGVAM | 353924 | YKNVE | 375145 | NPNIL |
| 263309 | ADVDH | 318409 | TAIVL | 353928 | EFMAS | 375146 | LPIRN |
| 263311 | HRLVF | 318415 | GRSRY | 353931 | DVFQR | 375147 | KPANC |
| 263314 | FSIGH | 318417 | RGSAL | 353940 | SGQQV | 375148 | KATLC |
| 263317 | KESFS | 318421 | LFQLV | 353942 | QKIFA | 375149 | QPACC |
| 263321 | YQSHL | 318423 | TMNTL | 353945 | PKAYS | 375150 | QHSCC |
| 263326 | SEVSD | 318429 | KDSRG | 353947 | GSPPP | 375151 | GSSCC |
| 263331 | KLDVV | 318437 | LPSEK | 353950 | PGENL | 375153 | RHCGI |
| 263334 | AFDHL | 318445 | IFKGR | 353953 | TKKDS | 375154 | RQRVQ |
| 263339 | LENQA | 318451 | GIRLT | 353959 | AKRMK | 375157 | PQSSS |
| 263341 | QFVSS | 318472 | NESKA | 353967 | TEFKK | 375160 | LMARE |
| 263346 | EGEWD | 318479 | DNDYN | 353968 | NTYHT | 375162 | SVLRK |
| 263354 | EEDLR | 318484 | GMLRS | 353969 | TENSE | 375166 | RSEGS |
| 263367 | GTSTQ | 318486 | LGLDR | 353971 | PYNGT | 375170 | QVFKK |
| 263368 | SHQYQ | 318500 | HHAFR | 353987 | LPLPA | 375172 | DSPGG |
| 263369 | DFYCQ | 318502 | RSSPE | 353988 | ILHTF | 375174 | LAQYL |
| 263370 | GLAQS | 318516 | RECEV | 353998 | QPKQD | 375178 | IWERT |
| 263372 | ASIPR | 318520 | TSLKL | 354003 | ETSDQ | 375180 | WPGWS |
| 263373 | SGRRK | 318534 | MAPPS | 354019 | RSLFN | 375181 | TMDQI |
| 263377 | EENLF | 318546 | VESPL | 354024 | KEQKT | 375184 | AWPTW |
| 263378 | GGGAP | 318557 | ITIYS | 354028 | SPGSQ | 375188 | HLSSP |
| 263379 | PQVLA | 318565 | LPRGC | 354031 | ASFSF | 375189 | IFLVG |
| 263382 | SMDCI | 318602 | EKYRV | 354033 | VPPLT | 375190 | QGRFQ |
| 263383 | GSIAV | 318604 | PVICI | 354039 | PYWTN | 375191 | VYICK |
| 263384 | VSWTK | 318607 | GTEEV | 354044 | NDIPY | 375192 | SVKKP |
| 263388 | RQVLA | 318615 | SMASR | 354053 | LNHHS | 375193 | LEALE |
| 263390 | YVCLD | 318629 | FHAPL | 354061 | KDLFG | 375194 | LPKLV |
| 263401 | AVASK | 318631 | PAQAV | 354069 | HGSCL | 375195 | YRGGP |
| 263405 | IYDND | 318635 | TGGVY | 354070 | EAIPQ | 375196 | QRTRG |
| 263408 | FPNEK | 318641 | GSDSD | 354075 | RIWSR | 375197 | DGGDI |
| 263409 | NKPND | 318646 | LGFFF | 354080 | LRPYK | 375199 | LLTSQ |
| 263413 | GKCLA | 318650 | DSDKQ | 354086 | LHGAA | 375200 | TEKRR |
| 263431 | PVPVM | 318674 | CLVIE | 354087 | SLMAV | 375201 | LLTSQ |
| 263433 | SKLSK | 318684 | LPMVV | 354089 | SIITY | 375202 | AGLSR |
| 263437 | HDFMI | 318687 | NMDLD | 354090 | AERPG | 375203 | RKHPS |
| 263440 | QKKIS | 318690 | LIKED | 354098 | PRLAG | 375204 | GESGH |
| 263447 | ITNID | 318697 | EEIDG | 354099 | TCASS | 375205 | PNTFF |
| 263461 | EPIEE | 318713 | AILQK | 354107 | VETMM | 375206 | NCCIK |
| 263464 | RTFLS | 318727 | KIDDA | 354108 | YSRTM | 375207 | GVQWH |
| 263468 | CRDKR | 318753 | TSGDY | 354109 | HSSQK | 375208 | GLGNL |
| 263498 | PVFGA | 318770 | INKKE | 354111 | TDGFN | 375209 | ESKQK |
| 263511 | GCRSS | 318775 | PELDS | 354130 | TLSRP | 375210 | SCGLV |
| 263512 | LFPVP | 318784 | CCSEN | 354140 | LDHLP | 375211 | NKLKK |
| 263519 | VETSL | 318799 | CTEKP | 354148 | LICTP | 375212 | RGHQL |
| 263525 | QSLQF | 318804 | KVLVF | 354151 | LRWVQ | 375213 | PARKP |
| 263528 | KNSSC | 318811 | KSSNT | 354155 | QMKGP | 375214 | DKIKA |
| 263549 | FSSRQ | 318821 | PSGKR | 354158 | LEESS | 375216 | LLESP |
| 263551 | QYPYI | 318834 | CPPSS | 354159 | KLVTA | 375217 | GNFNR |
| 263556 | LSELE | 318846 | MFRIV | 354168 | AGDSG | 375218 | QEPFC |
| 263559 | EQPEM | 318861 | KEFKF | 354170 | WSPTM | 375219 | MLLEK |
| 263563 | PEDLL | 318867 | LHPTE | 354171 | CSICC | 375220 | GVNLR |
| 263569 | NFEVI | 318868 | GLPDF | 354175 | SRNQE | 375221 | TAKAL |
| 263574 | EQMQI | 318870 | GREET | 354190 | NLLLL | 375222 | APPEG |
| 263576 | EKIDY | 318877 | EKASF | 354193 | TYRPV | 375223 | VCVYI |
| 263578 | ENNII | 318878 | YKKIS | 354198 | LSLLF | 375225 | SQAVC |
| 263579 | EAQQS | 318881 | REFSR | 354203 | KREKK | 375229 | SNTDS |
| 263593 | SNVAK | 318883 | ALRKK | 354204 | EDDPD | 375230 | GLWLS |
| 263598 | SPGSD | 318884 | ISMKT | 354206 | GGQER | 375232 | ASSCC |
| 263610 | AAQPR | 318898 | EKPCE | 354207 | LDILG | 375234 | ASSCC |
| 263620 | NNSLP | 318899 | LGFLR | 354213 | DFGWD | 375235 | KSMLQ |
| 263621 | ASRTH | 318900 | RNQNK | 354217 | RQTAG | 375236 | ASSCC |
| 263629 | WDPGF | 318902 | NEDME | 354218 | QRSKV | 375237 | RTSSC |
| 263635 | IESNV | 318912 | RASFH | 354227 | PSPGP | 375238 | RTSSC |
| 263636 | PSFHD | 318914 | LEGHF | 354228 | ASRRL | 375239 | CEPTC |
| 263639 | TVVFK | 318916 | VECSV | 354232 | AENEF | 375240 | HPEQV |
| 263640 | LKTDC | 318921 | TATKA | 354251 | VTSSA | 375241 | LAEFY |
| 263642 | FSDED | 318927 | SWWPL | 354255 | WLLKY | 375242 | HGIHV |
| 263650 | NHILK | 318944 | KVWTC | 354263 | ASFSF | 375243 | CSDNV |
| 263655 | KESFL | 318956 | PRKDM | 354265 | PDSDP | 375244 | YISPG |
| 263657 | SADRF | 318965 | VTEMP | 354266 | SSGLC | 375245 | TMGQA |
| 263663 | KVRRH | 318966 | KKDSK | 354270 | EEREL | 375246 | AAFGL |
| 263666 | SVTTV | 318974 | SHKSR | 354280 | IAANS | 375247 | NHKSM |
| 263671 | DITKT | 318977 | RFQST | 354291 | TVRRG | 375248 | GHPRS |
| 263672 | ERKIK | 318982 | ATLFF | 354293 | PNFSP | 375249 | SQPRK |
| 263674 | LLWRV | 318986 | STGKW | 354294 | ILAAN | 375250 | AYLKH |
| 263681 | FFQRK | 318988 | VRIDV | 354299 | LELKA | 375251 | KQTFI |
| 263686 | FDPSP | 318997 | IRQNS | 354305 | FRRDL | 375252 | WSRSR |
| 263694 | MGEIQ | 318999 | NKNSR | 354313 | SGGDS | 375254 | NVPNS |
| 263697 | MEQRE | 319006 | PYSAV | 354320 | TLLTS | 375256 | GGVHL |
| 263702 | QILTM | 319009 | SEEEM | 354335 | GQSPA | 375259 | GTRPS |
| 263707 | LKCGL | 319020 | VLHFS | 354337 | APAVP | 375260 | EAKSD |
| 263708 | TSTNK | 319027 | SYSQS | 354340 | LHFTS | 375261 | TAPTS |
| 263710 | VSTHS | 319037 | HQGLI | 354361 | IYKWE | 375264 | QALLG |
| 263713 | LTTEL | 319051 | LRTVL | 354368 | FADDP | 375267 | EDEDF |
| 263717 | TNIEN | 319052 | KTSLY | 354370 | PNGML | 375268 | EDEDF |
| 263723 | LSTTI | 319053 | THAGA | 354376 | CCISL | 375269 | FVLVI |
| 263726 | DHPPF | 319062 | GFLFL | 354386 | WAGEP | 375276 | KRDHP |
| 263733 | PLSHL | 319071 | TVSQN | 354394 | MMNTV | 375277 | KRDHP |
| 263734 | LDQAT | 319083 | LAFVC | 354403 | YTSAK | 375278 | KRDHP |
| 263735 | RELNA | 319090 | RENKL | 354410 | KDNRQ | 375279 | KRDHP |
| 263736 | LIRVL | 319091 | LVHIR | 354412 | CFFLR | 375280 | KRDHP |
| 263753 | HICLR | 319093 | IGFYI | 354414 | VPVLS | 375281 | KRDHP |
| 263754 | GLIDK | 319096 | ACVIL | 354416 | HKTPY | 375282 | LLVFY |
| 263762 | PSKDA | 319104 | DEMDR | 354421 | LPVKP | 375283 | SPRGM |
| 263765 | GLCGG | 319118 | EISPL | 354427 | RPRHT | 375284 | CPHEE |
| 263773 | MAPNT | 319125 | TYFYF | 354441 | SDDEF | 375285 | YCNPK |
| 263774 | KPDAK | 319126 | KDSRH | 354446 | TGEKP | 375286 | QTRRS |
| 263776 | SDQRR | 319139 | LGEDS | 354448 | AISCW | 375288 | LSMAG |
| 263780 | ALGVD | 319141 | QRSTM | 354451 | KFLRK | 375289 | AGDAK |
| 263791 | YRILF | 319152 | FRAIV | 354453 | QRTHT | 375290 | IPDVR |
| 263795 | NEKSC | 319165 | QMRSR | 354458 | QTQAC | 375291 | RRHPL |
| 263798 | PHSSC | 319166 | ESLYS | 354463 | RQAQD | 375292 | QHKDE |
| 263800 | PTYRS | 319169 | YTIGL | 354476 | AIAAS | 375293 | LLTSQ |
| 263802 | SKLSR | 319170 | CEKDD | 354478 | QPQLM | 375294 | LLTSQ |
| 263805 | SKIDS | 319179 | LQVYC | 354481 | GKLCS | 375295 | APCGQ |
| 263810 | SFAQH | 319192 | GEFIY | 354486 | RSGIF | 375296 | APLSN |
| 263812 | VAATQ | 319197 | SREDT | 354490 | KETYY | 375297 | FFLFY |
| 263816 | EDSEV | 319202 | IMLTP | 354497 | SQSLS | 375298 | LQEQV |
| 263817 | GSPIS | 319210 | SSCLG | 354498 | TQFMN | 375299 | IFIKL |
| 263826 | ASGRE | 319217 | SAQNF | 354499 | VYMKS | 375300 | DRRAP |
| 263845 | KLFPL | 319228 | QPEDV | 354501 | NQLLL | 375301 | LLTSQ |
| 263846 | HQLKA | 319231 | KPRTP | 354507 | GLPQH | 375302 | AVPPL |
| 263847 | CPDIF | 319233 | YEVIY | 354518 | KGALL | 375303 | PPTEP |
| 263849 | RRMIL | 319235 | HQTTR | 354519 | RIIQI | 375304 | SNQRM |
| 263851 | GTKEH | 319240 | KIKPI | 354522 | EDYEF | 375305 | HTTKK |
| 263856 | ATLRS | 319250 | ESSLS | 354525 | KVYWY | 375306 | VAELI |
| 263857 | KQPLR | 319251 | LKDLI | 354526 | SHNEL | 375308 | HCLPS |
| 263864 | TGAFS | 319254 | REACG | 354540 | IVHYG | 375309 | FGGQA |
| 263867 | FKDWK | 319255 | DATTD | 354541 | STTRV | 375310 | HVIWI |
| 263881 | HENSY | 319279 | RISEK | 354543 | NVSSA | 375311 | GWRLQ |
| 263895 | SCTVM | 319281 | VTVKE | 354545 | CVGAG | 375312 | CSLPL |
| 263897 | MPKLK | 319286 | ERALG | 354548 | PRPVL | 375314 | CHHTS |
| 263904 | QQPLL | 319288 | HRPAA | 354553 | YQSIN | 375315 | CSPPG |
| 263915 | ARIAL | 319305 | SNSSS | 354554 | NKMLK | 375316 | IIEVM |
| 263918 | AKVFV | 319318 | DIEDF | 354558 | WCPFW | 375320 | AEEDV |
| 263921 | IFRPD | 319321 | RWGLF | 354560 | YGYGS | 375321 | AKFLA |
| 263923 | SSPPV | 319322 | KRLCI | 354566 | NQRRI | 375322 | PTPFR |
| 263925 | PGTFL | 319323 | LRALK | 354568 | ENNFD | 375328 | LHRYR |
| 263932 | AASGK | 319326 | RPWRF | 354569 | SITSI | 375329 | STMYI |
| 263946 | FTSRF | 319336 | QVLRN | 354571 | ILGLE | 375331 | RFSEC |
| 263947 | HKMLS | 319341 | PKNCK | 354574 | TSVQV | 375333 | KESHS |
| 263955 | SDLLC | 319342 | SRHRR | 354575 | ESLVV | 375334 | KESHS |
| 263956 | TYCSI | 319343 | TSTDF | 354581 | QFNTH | 375335 | RTLSM |
| 263960 | EVHHT | 319370 | RAEKS | 354582 | SQPIT | 375336 | KERLT |
| 263963 | FNLDP | 319371 | RFYHK | 354583 | YESHV | 375337 | YGLCL |
| 263964 | LHLRK | 319388 | VTSDP | 354586 | LNMMT | 375338 | KQRSM |
| 263966 | RRIPS | 319393 | AKPKP | 354590 | SSLLS | 375339 | KKRTS |
| 263967 | QHALN | 319398 | KRRRT | 354597 | APPEC | 375340 | KQRSK |
| 263969 | SNEES | 319412 | IIPKI | 354600 | EIFFS | 375341 | ASRSD |
| 263974 | KTTKK | 319417 | SNKEN | 354603 | KQSQC | 375342 | MRHPK |
| 263980 | FPKGQ | 319422 | VIFPQ | 354607 | SLSHV | 375343 | GFWVA |
| 263985 | KKLSL | 319436 | TCDRC | 354608 | EPWLS | 375345 | KGQTL |
| 263991 | KCVLQ | 319464 | RAAGP | 354612 | PSTEL | 375347 | LCKAQ |
| 263997 | DLDSD | 319474 | TYRTF | 354614 | PNPQL | 375348 | QLFRN |
| 264005 | PPPPE | 319476 | TDVTL | 354616 | SQASQ | 375349 | YVKPK |
| 264009 | ASPSP | 319479 | ERLLM | 354620 | WDSIV | 375350 | QATSG |
| 264010 | SMMDR | 319482 | PEEQT | 354622 | IKDAF | 375351 | KQSDQ |
| 264012 | GGEDD | 319486 | LELLA | 354623 | FNVML | 375352 | HSPVK |
| 264018 | EDEKP | 319501 | KKPKV | 354632 | LHDNT | 375353 | ADFAC |
| 264020 | TLTFS | 319506 | NINIT | 354633 | KIIFF | 375354 | LWGVR |
| 264025 | KEWYV | 319511 | RTSFH | 354643 | EKLHS | 375355 | DGRGA |
| 264027 | FTKTE | 319531 | KSIEE | 354645 | PSLPD | 375356 | LSTFE |
| 264029 | SLLPW | 319546 | VERAL | 354647 | GGFTS | 375357 | PPKPM |
| 264031 | LGSRK | 319547 | LFLGV | 354649 | SFLMS | 375358 | LEMRL |
| 264033 | AHVAT | 319558 | GVVAR | 354652 | DNEKI | 375359 | KVTVK |
| 264036 | IDLRH | 319570 | KPCCY | 354660 | VSFSE | 375360 | WNAVA |
| 264037 | SGTTS | 319574 | TASQR | 354663 | SEAEK | 375361 | SPPST |
| 264039 | RPRWR | 319590 | HLSTL | 354665 | IARGN | 375362 | KELLY |
| 264042 | RGKEE | 319591 | KVSAL | 354669 | DCILL | 375364 | GGTEQ |
| 264047 | EDNLQ | 319597 | EESDS | 354671 | LLKQT | 375365 | GGTEQ |
| 264051 | SRNRQ | 319601 | NKHIA | 354675 | IIETM | 375366 | GWDEP |
| 264057 | PAVEA | 319622 | FCMEL | 354676 | GYLMG | 375368 | QCMKI |
| 264059 | ANFNT | 319623 | VSSSA | 354677 | KREDL | 375370 | RTGRA |
| 264065 | NKDEL | 319625 | TRCTL | 354680 | ERLSA | 375371 | LPGLR |
| 264071 | EEEVA | 319632 | RGERA | 354684 | FDFEL | 375373 | SALPF |
| 264072 | APLHS | 319635 | TSTTL | 354686 | SAQHH | 375374 | SLPKC |
| 264073 | NKSHK | 319636 | PIPQR | 354687 | IPPQT | 375376 | GLNRG |
| 264079 | SLLVN | 319651 | NQDNP | 354688 | QMLKI | 375378 | SLAVK |
| 264080 | GRELL | 319654 | LCEEK | 354698 | ERPKK | 375381 | FLCHG |
| 264088 | PQPPE | 319664 | KAKFN | 354700 | AKTLK | 375382 | LLKMI |
| 264089 | GSKDR | 319673 | KQMAP | 354703 | LGPDG | 375385 | CERPV |
| 264093 | FVKNL | 319678 | LHKKN | 354707 | LLGKG | 375386 | CERPV |
| 264094 | SNQII | 319690 | GEDDS | 354708 | ETPAP | 375387 | SEVIS |
| 264106 | NESYS | 319699 | AIEKM | 354709 | NETTV | 375388 | DNPPA |
| 264108 | RLAQE | 319705 | ITTNL | 354711 | LTANH | 375389 | ASTWV |
| 264110 | QPSGS | 319706 | EKQRG | 354717 | WIHFI | 375390 | AWLQS |
| 264122 | PRLNL | 319713 | DARGY | 354718 | EEIFC | 375391 | IEEVD |
| 264128 | GVTQK | 319716 | LSPGV | 354719 | KERGK | 375392 | PGKGL |
| 264144 | ALEQQ | 319726 | SLEQG | 354720 | DTTHG | 375393 | VPMEI |
| 264151 | LKMEI | 319730 | WLSEH | 354721 | TDAHL | 375394 | DNPPA |
| 264156 | YLLED | 319744 | LPPAQ | 354722 | DIDDI | 375395 | ACRVN |
| 264157 | QGMNM | 319756 | LGQRK | 354726 | FGVYI | 375396 | SFVDF |
| 264158 | DTSFF | 319771 | KSALL | 354728 | NLLQC | 375397 | ACSIM |
| 264159 | FLVKK | 319778 | YLRGK | 354729 | SNGSN | 375398 | VCIRG |
| 264160 | RASSQ | 319779 | APHFR | 354730 | EEQQI | 375399 | IEEVD |
| 264161 | KRLTP | 319788 | IKARK | 354733 | AQAPL | 375400 | PGKGL |
| 264162 | PVSSF | 319796 | KYPPV | 354734 | ELKIL | 375401 | AQIQP |
| 264167 | NRNLL | 319799 | QRAEA | 354739 | ECPAS | 375402 | NKMLK |
| 264169 | SLSWL | 319806 | KVLTW | 354742 | QTIGI | 375403 | IARGN |
| 264170 | AETKN | 319814 | RISQM | 354749 | PHREA | 375404 | LLLIT |
| 264181 | LQHSR | 319817 | PASEK | 354753 | PGNLV | 375405 | NLLQC |
| 264183 | SVECR | 319826 | LVRVE | 354755 | AQEIG | 375406 | LDWAE |
| 264187 | CIEQK | 319829 | AVGDN | 354762 | RASGS | 375407 | YVSIY |
| 264192 | EESRF | 319831 | PAPEN | 354765 | QRKQK | 375408 | PVFTL |
| 264193 | RDWVR | 319851 | LATQR | 354769 | DDCCC | 375409 | VYMKS |
| 264198 | PLYNS | 319853 | LGRKT | 354770 | SELDN | 375410 | MLMIL |
| 264211 | ESEDN | 319897 | YSPLP | 354772 | QIPQS | 375411 | LYEVM |
| 264218 | SAGFI | 319906 | LSAAT | 354774 | EQQIF | 375412 | GTHYR |
| 264220 | VELEW | 319910 | SLSDT | 354775 | IAEAP | 375413 | VPGCA |
| 264221 | RECNL | 319914 | SIKVH | 354777 | ENSKI | 375414 | RAPAS |
| 264228 | LPFLF | 319918 | HFDAM | 354778 | KEWLI | 375415 | FLVVL |
| 264229 | PQIIK | 319936 | PSIIS | 354780 | RRKML | 375416 | VNFQC |
| 264230 | GKINV | 319974 | SCADQ | 354787 | PHHRS | 375417 | RQRSF |
| 264231 | HTPEL | 319975 | QDKIN | 354789 | WMSRQ | 375418 | LVTVG |
| 264234 | SRIEY | 319977 | MEVSC | 354796 | HWTYK | 375419 | LVTVG |
| 264244 | KQDKD | 319979 | FLHFS | 354800 | KLIYH | 375420 | SFCYR |
| 264245 | GRQIE | 319984 | IGATR | 354806 | SLTEQ | 375421 | LYEVM |
| 264246 | SVRPV | 319986 | PPPPL | 354811 | DSRCN | 375422 | LVTVG |
| 264249 | DFLLN | 319991 | QMEPV | 354813 | LLLIT | 375423 | RQRSF |
| 264254 | DSEGD | 319992 | ELYAV | 354815 | ESQKK | 375424 | PLIPM |
| 264255 | DSDDD | 320006 | LMVLS | 354821 | VKSCL | 375425 | LVTVG |
| 264257 | TLTTG | 320017 | ISVGR | 354826 | SPTKV | 375426 | RQRSF |
| 264258 | NVDEN | 320025 | VYIRQ | 354830 | VIEGS | 375428 | LPRGC |
| 264260 | QPKEW | 320043 | LPPID | 354834 | KPWET | 375429 | LPRGC |
| 264263 | GKAKN | 320050 | HVRLH | 354841 | LQGLQ | 375430 | CEPRC |
| 264265 | EVQLE | 320053 | KQDEL | 354842 | ALYLT | 375431 | RTSSC |
| 264266 | FSHTE | 320077 | PSERE | 354846 | IRQRI | 375432 | RTSSC |
| 264276 | REKLN | 320081 | SNNVR | 354848 | KMALT | 375433 | RTSSC |
| 264279 | RENED | 320083 | LKEVL | 354849 | HNKDF | 375434 | RTSSC |
| 264312 | DTWDE | 320084 | GQEIA | 354850 | LGRSL | 375435 | ASSCC |
| 264313 | KDGCY | 320087 | RPKSE | 354852 | EDQLG | 375436 | ASSCC |
| 264316 | IAETW | 320089 | GVRAW | 354855 | KDRRK | 375437 | ASSCC |
| 264318 | SESLM | 320092 | VPLPQ | 354866 | FKHNF | 375438 | FSLWL |
| 264319 | VSTGF | 320096 | VEADQ | 354869 | ISALK | 375439 | RAPAS |
| 264326 | VDLPN | 320117 | RVTLN | 354871 | VTNLG | 375440 | NLKEC |
| 264331 | FAMFN | 320122 | AESHL | 354874 | GSFRR | 375441 | TGGKV |
| 264335 | EDENQ | 320130 | VELSA | 354876 | PVFTL | 375442 | CPTRI |
| 264344 | DSKSM | 320133 | GPFGS | 354877 | STATV | 375443 | GSGRR |
| 264345 | GFSLA | 320147 | EMEIP | 354880 | GNMFL | 375447 | PGYTM |
| 264346 | MLTQS | 320168 | FEPHI | 354883 | AGGAP | 375453 | PQSSS |
| 264350 | NRGFG | 320171 | VVPVP | 354884 | VLLST | 375459 | MTFSC |
| 264357 | ESVKI | 320176 | VKLLE | 354888 | KRRRF | 375463 | NVTFL |
| 264360 | RKRIC | 320180 | LTSMC | 354890 | FTSAL | 375464 | HASAS |
| 264363 | LQKVR | 320184 | GLLPP | 354891 | ISTDV | 375465 | LILLG |
| 264376 | VIDFS | 320188 | GGLAS | 354892 | AHTNK | 375466 | PTLGG |
| 264377 | QQGPI | 320207 | SYSVS | 354895 | ADDDP | 375467 | NSSLA |
| 264380 | LGLNC | 320210 | IPKDV | 354900 | RCSAC | 375468 | FMVIS |
| 264381 | SCVGL | 320218 | NRNFI | 354901 | QKKTT | 375469 | DLAHR |
| 264382 | EINWS | 320219 | GVTCG | 354903 | RGMGG | 375470 | VGFLY |
| 264387 | WFQGN | 320221 | CCVQE | 354911 | DENES | 375471 | QIGEG |
| 264389 | AQMAQ | 320232 | TVFHS | 354912 | VHNFR | 375472 | AWPTW |
| 264399 | WDKDF | 320234 | PWVIT | 354913 | LKYFQ | 375473 | TKSYT |
| 264409 | NGSLS | 320236 | DYYLR | 354916 | AKEAF | 375474 | LSPAG |
| 264412 | TLVLK | 320239 | VRLML | 354917 | ILNEL | 375475 | TPSCC |
| 264414 | YTYVA | 320246 | QSQLN | 354919 | VTTYL | 375476 | TPTGC |
| 264424 | HQNQD | 320247 | EMESV | 354920 | TVKIV | 375477 | QKSSC |
| 264426 | ESVKI | 320248 | VPSLL | 354923 | REDTM | 375478 | QKSSC |
| 264428 | WSIYL | 320251 | ALMGV | 354927 | AQLMY | 375479 | QKSSC |
| 264431 | QVSAV | 320252 | DLALK | 354929 | EHDEN | 375480 | GRHVH |
| 264432 | RIEEC | 320264 | LESAS | 354932 | EQAKK | 375481 | IPKMS |
| 264433 | AQILL | 320279 | SVCSL | 354945 | LNSLK | 375482 | FGYYY |
| 264434 | TITFP | 320291 | LPDIY | 354947 | EMQNA | 375483 | AHYLL |
| 264436 | EKVES | 320302 | KRKCC | 354955 | ERGML | 375484 | CGSHL |
| 264441 | LKTEL | 320303 | KIRSF | 354957 | FTQPS | 375485 | MGVPM |
| 264444 | ACLGL | 320309 | TQEAA | 354961 | TGFSS | 375487 | GGGQT |
| 264447 | ERSSR | 320318 | CCWPS | 354962 | DDMQL | 375489 | ELACI |
| 264448 | KVPWD | 320324 | SPPTV | 354964 | SSVHN | 375493 | PGRKH |
| 264451 | DLEIL | 320332 | SIQMK | 354966 | EQELL | 375494 | LVALE |
| 264452 | APTVP | 320337 | KRQKQ | 354968 | LLFRP | 375498 | DFLYF |
| 264454 | SDCGV | 320340 | QETAV | 354972 | VVKLV | 375501 | GAELT |
| 264463 | SDKDS | 320346 | KNRGS | 354982 | IYWWG | 375503 | EEDKK |
| 264474 | ELTGF | 320347 | VHCME | 354988 | SGGWC | 375511 | LVALE |
| 264485 | RHTSC | 320349 | VESGS | 355001 | QTSVQ | 375513 | LVALE |
| 264487 | VHAIA | 320352 | KSKNS | 355008 | DHADL | 375515 | YRAGM |
| 264492 | NGKSN | 320359 | MSIMT | 355011 | METQE | 375517 | LLTSQ |
| 264497 | GSEDS | 320376 | NITLG | 355013 | SDDSD | 375518 | LLTSQ |
| 264498 | MSAKS | 320378 | GQPQP | 355014 | FPPPL | 375519 | ELSQA |
| 264499 | FFDAA | 320390 | PLAIH | 355019 | LGSFL | 375523 | SGRRF |
| 264501 | DKEEH | 320396 | SKLHS | 355022 | DEPAV | 375524 | MSYPK |
| 264515 | ISELL | 320401 | KKKRD | 355023 | TKNNP | 375526 | LTFSP |
| 264538 | ATGFY | 320408 | TVWDG | 355026 | NAPPP | 375529 | LAWQT |
| 264546 | PGTLV | 320413 | SCSIP | 355028 | QLPKI | 375532 | LVRPR |
| 264552 | ALRRL | 320416 | EPPHR | 355033 | ESTAE | 375533 | RERPK |
| 264553 | TGRSA | 320430 | KKKGK | 355041 | EDGTE | 375534 | ACWEM |
| 264554 | VSREP | 320431 | LSHCA | 355046 | MLMIL | 375535 | GLLTS |
| 264555 | QGAEG | 320434 | MDSKT | 355050 | LLENF | 375537 | RQRVQ |
| 264563 | LKTRL | 320447 | TGASL | 355051 | ANYGS | 375538 | VGVYY |
| 264568 | QDIKL | 320465 | FTETG | 355052 | SMAQP | 375539 | RAATE |
| 264595 | KVDCI | 320468 | PSVQG | 355060 | VPLML | 375540 | PARFA |
| 264596 | IIPGC | 320485 | LIGQS | 355064 | SYPGA | 375541 | AVFSG |
| 264597 | ITKSY | 320488 | DDAKK | 355076 | GATCH | 375542 | NNGCQ |
| 264601 | KCCAH | 320491 | WCTVY | 355077 | RSRQH | 375543 | FGLFD |
| 264605 | VAHQS | 320503 | PLHSA | 355082 | DVRQS | 375544 | CQNRF |
| 264606 | EEPPL | 320508 | WLLIA | 355086 | AERAH | 375545 | VTLSI |
| 264607 | FLLHE | 320509 | CKKDH | 355089 | ANRPY | 375548 | QWIPL |
| 264613 | DTKSG | 320510 | NSNST | 355090 | KPVDL | 375550 | FFFSF |
| 264634 | QFVCK | 320516 | KRRHE | 355092 | VALLL | 375555 | NWGER |
| 264637 | NAASP | 320520 | ACCAR | 355094 | AATGN | 375556 | PALPT |
| 264638 | ESRSE | 320532 | SMLNP | 355108 | KYRLV | 375557 | PMHSN |
| 264639 | DDSFP | 320548 | RGAGP | 355109 | AQYAN | 375558 | PPKPM |
| 264640 | PSTAN | 320560 | FSHRQ | 355110 | VGRLV | 375560 | VPGPP |
| 264644 | EANSQ | 320566 | GSRHK | 355114 | AGLWS | 375561 | EPDQI |
| 264645 | SRGSS | 320573 | ADQGC | 355119 | QEPNP | 375562 | CPSQA |
| 264646 | ETDVL | 320604 | PKRME | 355121 | LAAAP | 375563 | IGPLW |
| 264649 | GKFEE | 320627 | KIHMG | 355124 | ASKVL | 375564 | FKGIL |
| 264651 | EVKVK | 320634 | MESVL | 355133 | DEIAV | 375565 | VYSLP |
| 264657 | ATSPM | 320646 | HFHPS | 355136 | KPLYL | 375566 | KLHVL |
| 264658 | CCIIL | 320648 | GALWS | 355140 | SSVTS | 375567 | ATVIH |
| 264659 | SSISF | 320649 | RHLSP | 355146 | GDQEN | 375568 | DVYQE |
| 264661 | SDTFH | 320650 | TFVDD | 355153 | ARGPG | 375569 | SDIDI |
| 264663 | ESYQK | 320657 | GVCGQ | 355155 | RTCES | 375570 | LALPR |
| 264664 | MVVHS | 320658 | KWLQL | 355156 | SFREK | 375571 | LAEFY |
| 264668 | QDIWS | 320663 | PSPSS | 355162 | SLSPK | 375572 | HTHMC |
| 264669 | HWLQE | 320664 | SWFGC | 355166 | PPTNS | 375573 | KVGAG |
| 264670 | VHPPR | 320672 | FAGKI | 355167 | PKEHI | 375586 | SLKLV |
| 264674 | SISHV | 320675 | YENTQ | 355170 | DDTAL | 375592 | ESPLA |
| 264677 | DLDSL | 320679 | CCSSK | 355173 | QVHKV | 375613 | TLAIH |
| 264689 | RPNMI | 320688 | YLLNG | 355177 | GSSQN | 375622 | AVARH |
| 264690 | MQSPA | 320709 | YHDTN | 355178 | IKNIQ | 375629 | TLAIH |
| 264691 | KTQAV | 320718 | ALPKH | 355180 | KVALG | 375653 | LDIGC |
| 264694 | KAGQV | 320721 | QGSID | 355185 | LEAGY | 375660 | PSSIN |
| 264703 | NTIDV | 320754 | TKKKL | 355192 | IPPRL | 375675 | IKIHK |
| 264705 | VLGRF | 320757 | LPVQL | 355193 | EHIEL | 375682 | IGSKG |
| 264708 | YKKGE | 320768 | PIHLS | 355198 | SSSHA | 375690 | FFLER |
| 264709 | YFACV | 320783 | PQAPS | 355203 | APCLL | 375692 | PFTQE |
| 264710 | KSKCC | 320794 | CSSAV | 355204 | KVSHV | 375693 | GPEAW |
| 264711 | LKNIK | 320796 | VNTCG | 355205 | PSGGR | 375711 | LKRKY |
| 264712 | VADHE | 320797 | SYCNL | 355206 | LDWAE | 375736 | YTSSK |
| 264714 | KAKRD | 320813 | NRTKP | 355216 | KTGCD | 375751 | QRSLA |
| 264716 | TLLAL | 320821 | SGAAV | 355222 | IAANS | 375761 | LEAKV |
| 264717 | EPDVQ | 320828 | RGEDS | 355228 | AGDAK | 375767 | RQQNL |
| 264718 | KRNNK | 320838 | EEEQC | 355230 | SLAAK | 375770 | GPLQW |
| 264730 | NQDST | 320848 | EPRPQ | 355231 | QFYNK | 375773 | ELSSY |
| 264731 | KEEGE | 320849 | GEASL | 355235 | LVNDT | 375777 | AKVFV |
| 264734 | VDTRV | 320852 | IGCGR | 355237 | PACDT | 375783 | VMTTV |
| 264735 | RAKYY | 320853 | GTPLP | 355241 | CPSQQ | 375809 | IAQQL |
| 264741 | VQKNQ | 320866 | AKDEL | 355245 | TASAL | 375815 | PHYRH |
| 264748 | IHYQC | 320869 | IKLCP | 355249 | DLLIN | 375820 | SSESP |
| 264753 | VTEGK | 320871 | TKQSI | 355250 | LEAAT | 375826 | LEVDV |
| 264758 | KKSDS | 320885 | GDTTV | 355257 | QCIPS | 375829 | LDAEV |
| 264771 | LPAPC | 320886 | LGKPL | 355260 | QKKSV | 375833 | LGHHV |
| 264779 | DDDDV | 320893 | LLLLE | 355261 | WKEIG | 375844 | AVMKT |
| 264784 | INGRP | 320898 | QRCTK | 355264 | SDEDL | 375849 | PYQED |
| 264790 | LLYRT | 320911 | FRYNK | 355265 | LPPQS | 375857 | DCTMS |
| 264808 | GVADS | 320917 | PKNSS | 355271 | RGLLT | 375869 | TRREV |
| 264818 | VFSVC | 320919 | RGLFS | 355273 | VIPHS | 375873 | LWAQY |
| 264819 | NVMTC | 320924 | PPILR | 355279 | ANKQS | 375892 | ASIRE |
| 264824 | SPEVR | 320935 | PDEND | 355280 | YCGNV | 375895 | GEKAS |
| 264828 | VCFSS | 320936 | EGPSA | 355293 | EAKSD | 375909 | LLELG |
| 264832 | QATPP | 320939 | TQSTT | 355296 | HTINL | 375913 | RLRTF |
| 264833 | TSGDP | 320940 | QLLTE | 355299 | ISLRR | 375924 | GDLCV |
| 264834 | MKRHL | 320949 | GTGAS | 355304 | RKGGR | 375927 | RIRLS |
| 264839 | PCIRS | 320951 | CRRKK | 355305 | KSPEV | 375930 | EETKS |
| 264848 | LLLYE | 320952 | HTAGC | 355306 | DDAEM | 375931 | REKPQ |
| 264864 | FFSSW | 320957 | ASLLS | 355308 | VEKDD | 375932 | PQPCG |
| 264866 | FATVT | 320965 | LRGSH | 355315 | TKLGL | 375968 | SYMHS |
| 264867 | RSLRR | 320968 | MEVLI | 355316 | TTSSL | 375986 | CTDEE |
| 264868 | PEPRG | 321005 | KFVSA | 355319 | DHPAI | 375987 | QRIHI |
| 264870 | RRPSM | 321014 | VSFWW | 355323 | PADGP | 375989 | SRSHL |
| 264874 | TKKGK | 321015 | PGEVP | 355324 | LHQEK | 376007 | GRSGT |
| 264883 | GGVFS | 321017 | PSQRS | 355325 | SGSTP | 376010 | QNLYW |
| 264893 | NASFT | 321019 | RKRLR | 355331 | DTSLL | 376015 | GYLMG |
| 264896 | PLIRT | 321021 | FDSDD | 355339 | RTQVS | 376023 | GLPPV |
| 264903 | LPNRR | 321026 | YYCFG | 355342 | DCGQF | 376037 | FPGKI |
| 264904 | DLDHI | 321029 | FLNLP | 355343 | SWGAN | 376045 | KAILS |
| 264908 | CGGDD | 321038 | VPKTD | 355344 | FSCPE | 376065 | RRETV |
| 264914 | WGPWM | 321040 | FSWFT | 355345 | KCHCN | 376076 | GHSTV |
| 264917 | LPSDS | 321049 | TGKKL | 355351 | YQCKI | 376090 | ELEKQ |
| 264926 | KRNRN | 321058 | EATTD | 355353 | ASQQL | 376103 | IQYDD |
| 264930 | ITIYS | 321070 | PVITE | 355354 | PLTHL | 376110 | ETVQK |
| 264932 | AIRSY | 321077 | KEVRG | 355361 | MMNDE | 376115 | ENGKL |
| 264935 | GCQAC | 321104 | GGSRL | 355377 | PNYFR | 376123 | LGPPQ |
| 264938 | ESTHM | 321106 | YERRR | 355378 | KVTDL | 376127 | LASLL |
| 264951 | SKPSE | 321108 | GGYIW | 355380 | AVALE | 376139 | TDDKL |
| 264952 | VCLLL | 321132 | HLIGY | 355384 | ALLKA | 376148 | GNLAP |
| 264954 | VVKEA | 321133 | HFLLS | 355385 | LEQHS | 376168 | LLLDF |
| 264956 | RRSNL | 321173 | QPLTP | 355387 | YSNEM | 376170 | GLSQY |
| 264968 | KKATN | 321175 | IASLI | 355388 | RVQLQ | 376176 | EDDDL |
| 264972 | EAACA | 321179 | ALSLF | 355396 | GSEGT | 376177 | ENLYN |
| 264977 | SVDEE | 321184 | PGAID | 355398 | AMDSF | 376188 | RGPAR |
| 264982 | KVLSY | 321195 | DSSTC | 355421 | KQSQC | 376200 | LRGGV |
| 264992 | ASNIE | 321196 | QDVSK | 355428 | RSVSL | 376213 | HWPGI |
| 264993 | HSQYN | 321201 | IAAPT | 355429 | VRFVL | 376214 | VLSIS |
| 264995 | SITFA | 321203 | AECCQ | 355430 | SGGVF | 376221 | DSQKF |
| 264998 | TFRRP | 321209 | KQPTS | 355431 | QKAME | 376227 | LSDDD |
| 265000 | NIHSV | 321219 | QKIHQ | 355432 | GKCGE | 376228 | PQLCL |
| 265007 | VLRDL | 321222 | DLREL | 355435 | FSVKM | 376240 | HATEP |
| 265012 | PSWYF | 321239 | SLDQQ | 355436 | FRSLY | 376246 | DDKFF |
| 265014 | RPSHN | 321242 | KKIYC | 355437 | RDDHL | 376247 | STKRH |
| 265018 | KYFSF | 321246 | LQRGR | 355438 | KLTKK | 376250 | FHYKH |
| 265022 | SRSKD | 321249 | DKRAS | 355440 | VMKRD | 376261 | SHISI |
| 265023 | TDGLS | 321259 | AENGK | 355441 | EAYFI | 376265 | SELKT |
| 265026 | SSATW | 321260 | YRRPE | 355442 | RQCGP | 376268 | RWRFC |
| 265028 | GLQGY | 321266 | AASDE | 355444 | VVFPH | 376295 | SPVLW |
| 265029 | LVLPP | 321271 | ASLGK | 355448 | TRGTL | 376298 | RQRTY |
| 265036 | RSFRM | 321303 | AMKKL | 355454 | ALNSY | 376302 | DGFQL |
| 265038 | SDEEG | 321309 | SESTD | 355455 | KCKKM | 376303 | QQAAA |
| 265044 | STGSK | 321316 | CPASC | 355459 | RAYMW | 376306 | DQSSV |
| 265050 | LLVSC | 321320 | RATPT | 355467 | SSSEV | 376309 | GSRRF |
| 265052 | TASPP | 321326 | KKLLT | 355470 | WIYLS | 376316 | WFRSR |
| 265056 | ILQQF | 321330 | DPLPT | 355471 | TLEDR | 376322 | KDTVS |
| 265068 | WPERP | 321334 | MMRNN | 355475 | TSRRR | 376328 | MALCH |
| 265069 | DYDNF | 321338 | NNLCD | 355479 | LRRPG | 376333 | NGGAE |
| 265070 | AAFTK | 321342 | QPEST | 355482 | HQGYY | 376350 | SQTVT |
| 265071 | SDKDS | 321343 | TCSCC | 355484 | QKYTV | 376352 | TFRSA |
| 265073 | AVRKL | 321345 | SLLEK | 355486 | GSSSN | 376354 | KKGKQ |
| 265074 | HFSVA | 321347 | HYSFF | 355492 | KRKKN | 376361 | LPHEA |
| 265077 | QESRR | 321354 | NLSVD | 355493 | MIQLF | 376370 | EDTEL |
| 265080 | PRLPA | 321360 | QMSQM | 355499 | PQSDC | 376372 | CVWQG |
| 265081 | TSLLH | 321365 | NFDDE | 355503 | DPRNV | 376375 | QKSFR |
| 265085 | SFRWN | 321370 | PFHFV | 355510 | PAIPK | 376390 | CCSFM |
| 265087 | SDIRR | 321372 | GGCPG | 355511 | KHLQN | 376394 | PETND |
| 265093 | KYHWP | 321377 | RVFMS | 355512 | QVRPL | 376395 | APPGR |
| 265097 | LPNDS | 321386 | VYSVM | 355517 | NLISR | 376409 | HYNGL |
| 265100 | EKMQE | 321388 | EGIIY | 355518 | MLGPK | 376410 | LCVIQ |
| 265104 | LCDVK | 321389 | KAKGK | 355520 | LAQSY | 376423 | PPKSK |
| 265107 | KKRKI | 321398 | EVHDD | 355527 | DQEET | 376426 | GGCRL |
| 265109 | QSQKK | 321406 | RTHTG | 355529 | DQMCQ | 376436 | PDADE |
| 265112 | AEEEF | 321410 | LEGCR | 355533 | EDQLN | 376440 | YFCII |
| 265113 | SETKM | 321419 | KLFTW | 355535 | GYDTD | 376443 | DRVVF |
| 265131 | RRKRA | 321424 | NATRL | 355536 | MPRKQ | 376453 | CSMDD |
| 265132 | LRFSN | 321426 | FVQKE | 355537 | GESDL | 376457 | TTHLI |
| 265138 | SCPSR | 321427 | DEIHF | 355541 | LQSYF | 376458 | QPRET |
| 265139 | KHEEL | 321445 | CGTTA | 355551 | KPSLG | 376469 | KRKKT |
| 265140 | VMEFS | 321447 | SLGEK | 355555 | AENPP | 376483 | AEECV |
| 265148 | ECKTQ | 321449 | KFRPY | 355556 | FCTIL | 376486 | FGCSQ |
| 265154 | IQSLK | 321455 | ELHSR | 355558 | RCQAR | 376489 | IQARG |
| 265162 | LLESG | 321468 | VQSRC | 355560 | LLVRW | 376514 | ALIVE |
| 265164 | FPKSN | 321474 | PTSRP | 355566 | EDDVE | 376515 | AHSQS |
| 265165 | TAAYI | 321475 | TSGLP | 355568 | KERDS | 376516 | AHSQG |
| 265168 | FYKYK | 321482 | KKTCL | 355574 | TTAKS | 376518 | AHPQR |
| 265171 | MELTQ | 321498 | HILRE | 355577 | PSFAR | 376519 | WSKST |
| 265174 | SLEKA | 321503 | VLLGS | 355580 | GPANH | 376522 | YQGRR |
| 265175 | QQICK | 321504 | ELEEI | 355583 | KEFLS | 376528 | GFAVE |
| 265191 | VEEPY | 321506 | GGKVI | 355586 | LRWIP | 376534 | TFKVS |
| 265192 | KYGNI | 321507 | FSAEA | 355590 | PKHKA | 376544 | HPGQE |
| 265193 | KGKDI | 321514 | REWYV | 355591 | ISSCR | 376551 | DMPAA |
| 265195 | LKELR | 321517 | ICLRS | 355598 | HEAQA | 376557 | TVFKI |
| 265198 | VENSI | 321519 | KMEDL | 355599 | EEGIS | 376564 | ASDLL |
| 265220 | LESLV | 321537 | AILIK | 355601 | GKDVF | 376570 | MTLGT |
| 265239 | RGKKK | 321544 | EQGAP | 355607 | ATAKL | 376572 | ACSIM |
| 265245 | KHLDM | 321546 | RELRL | 355609 | SRVAK | 376582 | SSDTD |
| 265260 | HDQDN | 321549 | TYCVT | 355613 | VTSLF | 376588 | PLRPC |
| 265261 | NLTQD | 321556 | RKADL | 355614 | ILESI | 376602 | GKADL |
| 265271 | RSWRR | 321573 | FKEIL | 355621 | CLKKL | 376611 | EESHA |
| 265276 | SFVVL | 321583 | NNYGI | 355623 | FLDNW | 376615 | EAQAK |
| 265294 | YYMYF | 321584 | EKRLF | 355627 | PLSTA | 376623 | GSCGF |
| 265295 | QCPQQ | 321594 | ETLDT | 355629 | TAEQP | 376630 | SCGCP |
| 265296 | FDHLR | 321606 | ITSLG | 355631 | LHVCL | 376633 | SKLAT |
| 265299 | SDPIL | 321617 | GRPSS | 355632 | LNLQQ | 376634 | TQMPH |
| 265304 | TKEKE | 321625 | SHCTV | 355637 | SFISA | 376636 | ARDPK |
| 265306 | EKNAK | 321627 | VACFT | 355645 | HRKCF | 376644 | TLIDL |
| 265310 | EVYHF | 321630 | PIRRL | 355649 | QAAHF | 376652 | GISTT |
| 265316 | QTMER | 321635 | SCLRH | 355651 | QECGC | 376667 | ISLLK |
| 265322 | EKAKL | 321636 | CPQNR | 355652 | YCCFV | 376684 | GSVEV |
| 265333 | LEFQA | 321641 | MDTAL | 355653 | PEPFL | 376694 | PSTVN |
| 265334 | RFFFW | 321649 | ARWTP | 355656 | KAKGK | 376704 | KLHQP |
| 265335 | GFNVH | 321656 | KDMSP | 355657 | RGERA | 376705 | HNEWP |
| 265339 | SWNDS | 321674 | MKENY | 355670 | LAQVR | 376710 | SKKCF |
| 265340 | CQYNS | 321679 | PRTRC | 355672 | PTVSK | 376719 | WYNKT |
| 265342 | WVGEV | 321684 | SPSSE | 355673 | IPHDA | 376722 | KVRPA |
| 265343 | AKLIS | 321698 | LVPSP | 355675 | TTVWI | 376729 | YCVLQ |
| 265344 | NDFQD | 321706 | KMSSF | 355680 | WNILG | 376734 | KKKKR |
| 265348 | FSTFR | 321715 | EASTK | 355692 | TRTKK | 376768 | SSEDD |
| 265350 | VAGIR | 321729 | SLKRY | 355712 | QWRRY | 376776 | GGGKG |
| 265351 | TIFEP | 321732 | QHRLW | 355715 | AGKVP | 376781 | RPPHS |
| 265354 | STKSE | 321735 | AAESV | 355718 | IATEV | 376788 | CRRAY |
| 265362 | APRSV | 321737 | LVKLA | 355719 | GWSVL | 376792 | SVRRH |
| 265371 | TYSEA | 321744 | YTSSK | 355720 | AAAAP | 376795 | NPKQA |
| 265381 | QPVYI | 321746 | HSVNF | 355721 | MKRLT | 376798 | EKMLD |
| 265394 | DPYVG | 321761 | LDMEA | 355728 | GSWDP | 376822 | KTSHV |
| 265395 | EEETF | 321797 | APEPR | 355730 | LVGKK | 376827 | GTLVS |
| 265403 | KGKRD | 321798 | ISVCC | 355735 | SKVLL | 376832 | REESG |
| 265404 | NPHIA | 321804 | ACPRD | 355736 | ALYYE | 376848 | NNAVV |
| 265417 | SSLLN | 321805 | RENMT | 355739 | RQAQQ | 376849 | LFPGN |
| 265421 | KDRSE | 321809 | HQHKS | 355746 | HQLYI | 376855 | PEEDE |
| 265424 | TRLCF | 321810 | LKCSV | 355751 | ECRDI | 376863 | RFTSS |
| 265428 | GFGQE | 321813 | PGLAT | 355752 | IPQQL | 376865 | SRPPR |
| 265431 | CAGDN | 321821 | NVSLQ | 355759 | KTSLW | 376867 | AKGIF |
| 265433 | LKRRR | 321826 | KWYQF | 355760 | VMTMV | 376870 | ASPHS |
| 265437 | QHFQN | 321834 | SKATF | 355765 | SSHEA | 376879 | PTPKS |
| 265440 | DGDEL | 321835 | GGYSY | 355767 | TLQGE | 376894 | DWVYE |
| 265441 | WTTAT | 321844 | SPTHC | 355772 | NTNRD | 376898 | MPGRM |
| 265447 | CGGND | 321845 | RQLLS | 355775 | AFGNF | 376910 | PSKLI |
| 265450 | AGHHF | 321874 | QTQIY | 355777 | VYYTR | 376937 | PGHSL |
| 265459 | KEYYV | 321876 | PIIQK | 355778 | RGERA | 376946 | SEDEL |
| 265462 | IISQL | 321891 | RIHSR | 355785 | RRLQP | 376952 | LDSAD |
| 265465 | QVVRI | 321903 | DAYTA | 355786 | SFCYR | 376956 | LVAML |
| 265471 | PAIEV | 321936 | QTQTY | 355789 | EEWVL | 376966 | IPSIL |
| 265498 | LRRQF | 321951 | GTNRE | 355790 | LKMLS | 376968 | IEATA |
| 265500 | RNGLE | 321962 | DYLKV | 355799 | RDSYS | 376996 | WSGLR |
| 265512 | TILIF | 321963 | TPEAS | 355800 | APQEA | 377007 | LYPRC |
| 265517 | SSGWF | 321971 | LVKSQ | 355810 | RRFIT | 377011 | KIYEV |
| 265523 | CCSRK | 321973 | HHLPI | 355812 | LEDVA | 377012 | QIQRM |
| 265526 | LAVMQ | 321976 | SPGGP | 355815 | AESDS | 377021 | CGKDI |
| 265529 | QAHRK | 321984 | KKIIY | 355825 | ALIND | 377042 | FIVRI |
| 265537 | FLVQD | 321988 | FRSEL | 355827 | LRNRK | 377043 | SFLPT |
| 265538 | GCLVL | 321997 | SDNSH | 355831 | TSTRL | 377054 | EVEER |
| 265560 | SMDTN | 322007 | GAKPG | 355837 | CCVLV | 377055 | AGKQH |
| 265562 | TLNKT | 322016 | SDLSA | 355840 | QNRGY | 377061 | MALSS |
| 265563 | GNLGQ | 322020 | QDPQA | 355843 | MEEDP | 377069 | LTYFP |
| 265564 | VGFLG | 322049 | GNTVV | 355865 | HWFDV | 377073 | TFTWW |
| 265565 | LEKLD | 322057 | DAFLG | 355866 | VETVV | 377074 | QLAAA |
| 265572 | MNKPV | 322061 | AAETQ | 355867 | RKETA | 377076 | LFFFT |
| 265577 | ELLNA | 322087 | HVLDV | 355870 | ALGCL | 377086 | RERNN |
| 265579 | YFPSG | 322088 | IHTRF | 355871 | STYYL | 377094 | SSTAS |
| 265593 | DDKCQ | 322097 | CAPFP | 355877 | YLLGQ | 377102 | SAERS |
| 265594 | KRESE | 322122 | FTMAL | 355880 | VYRMV | 377137 | SEDLE |
| 265598 | GYQRI | 322133 | TDEDD | 355884 | NELKL | 377141 | QLNNR |
| 265603 | FYTSA | 322142 | KRKKK | 355890 | FGRSY | 377142 | TIPEK |
| 265605 | ITVKH | 322156 | LRRRF | 355893 | NRTHF | 377147 | KRIIF |
| 265614 | FLQKF | 322159 | EFVLL | 355895 | MEKQK | 377148 | PQSWQ |
| 265616 | KEQTN | 322161 | QRCWC | 355896 | SCKCS | 377157 | IFNIF |
| 265619 | GKIAL | 322170 | RKKGS | 355899 | SDSDT | 377176 | CPLDV |
| 265627 | LYCEL | 322180 | RRPIS | 355900 | AGQIV | 377187 | LNTKL |
| 265634 | RLLDL | 322191 | NRTKP | 355902 | AGKSA | 377191 | YSTCL |
| 265641 | SNSKK | 322192 | LAPVA | 355906 | REEKC | 377195 | FEGGD |
| 265643 | DIERS | 322206 | IRIFS | 355908 | DRFYF | 377197 | KGITL |
| 265651 | CSRLF | 322218 | AAGPK | 355910 | TDTHL | 377204 | TSSWR |
| 265686 | AATDD | 322234 | DFTER | 355917 | IENIK | 377206 | TPDLR |
| 265689 | RKLGV | 322238 | TAGKD | 355920 | DIPLN | 377225 | KYTIR |
| 265707 | DDVGH | 322242 | VVHTK | 355922 | NCKVQ | 377233 | LLRNV |
| 265708 | SEPKG | 322249 | ELQAV | 355923 | NSRLI | 377244 | QEPAL |
| 265713 | PYMRR | 322252 | EQRDV | 355924 | EIESH | 377252 | QNGES |
| 265715 | RTLAS | 322265 | TPVQA | 355925 | FNNSF | 377256 | FPAPL |
| 265717 | VEPTA | 322273 | KEEEV | 355927 | AELMR | 377263 | KEQIP |
| 265720 | DHYGI | 322276 | NIVML | 355929 | FRLEL | 377265 | EKHRK |
| 265723 | GTQNL | 322280 | ADLMY | 355930 | EPSGT | 377279 | KDFDV |
| 265724 | GTKRQ | 322287 | PLRGA | 355938 | SESAI | 377280 | GHVAA |
| 265727 | WETSI | 322295 | HSIDK | 355941 | HLLQK | 377297 | SLPGH |
| 265728 | TFTGF | 322300 | KTSKT | 355944 | INLDT | 377303 | CRGAE |
| 265732 | QRRRI | 322304 | YRLIG | 355955 | IKDEF | 377311 | VPKPH |
| 265734 | ELNTA | 322316 | LSERL | 355958 | HSTEL | 377337 | LGGFS |
| 265742 | QVHLV | 322323 | FKPMK | 355961 | AYTRF | 377339 | SPGTP |
| 265753 | QKEQE | 322339 | LVVMP | 355963 | YHCLF | 377344 | KLNKV |
| 265758 | RKPRF | 322354 | PFPVT | 355966 | ILGMR | 377345 | LTGSF |
| 265769 | SNPKA | 322356 | LQSEH | 355968 | PESSL | 377355 | SVTTV |
| 265773 | GTDDE | 322373 | DDSSD | 355969 | APKAD | 377370 | AGSLV |
| 265782 | KKAKP | 322376 | KKNKH | 355970 | PATAS | 377372 | DQEHA |
| 265801 | IATEI | 322395 | GEGKK | 355972 | QSSLH | 377374 | LGKMH |
| 265806 | GPLPP | 322396 | GKRRK | 355979 | KGSKI | 377375 | HVSDG |
| 265807 | LELFE | 322408 | DEQQL | 355980 | QPAEE | 377381 | TPVEN |
| 265825 | CIWEF | 322417 | VVSHT | 355986 | ALLES | 377383 | PGHVD |
| 265827 | SKALV | 322418 | VLRSV | 355987 | ITGEK | 377385 | RELSQ |
| 265836 | HSAVL | 322419 | FGLFD | 355988 | ALPPQ | 377386 | TSLFP |
| 265838 | LIQKL | 322421 | TSKYR | 355991 | DNLMI | 377390 | VNGPV |
| 265840 | GLINM | 322422 | GLTTT | 355996 | RQHAS | 377393 | TYISR |
| 265843 | KESEL | 322424 | ISAPV | 355999 | PPPPP | 377401 | PVQKV |
| 265846 | FKHKP | 322426 | KLVLN | 356001 | QRIYE | 377407 | STGPL |
| 265849 | VISQN | 322427 | TGEKP | 356005 | LQAPG | 377414 | EWVCL |
| 265854 | PHLVK | 322435 | HSQPL | 356006 | VQLAL | 377416 | QIPVF |
| 265857 | PIELD | 322439 | NIKWG | 356015 | CVQRE | 377417 | QPPKW |
| 265866 | WRGMY | 322440 | EVTMI | 356017 | SRVLP | 377428 | QPADL |
| 265870 | FFHLN | 322450 | RQAAS | 356024 | YNPAS | 377435 | GQDSQ |
| 265872 | NGASV | 322453 | GTVCE | 356030 | KTLRL | 377446 | KRKML |
| 265881 | EKTVS | 322457 | STCCP | 356033 | KESYI | 377451 | VLAPY |
| 265896 | KYMVH | 322460 | PPSKV | 356037 | LDKEL | 377470 | SCTII |
| 265909 | MGVSS | 322469 | TGDQV | 356038 | GNTGQ | 377473 | SADDV |
| 265922 | TAKLC | 322498 | SEPSS | 356040 | PASTC | 377479 | QDRVP |
| 265944 | LVQQS | 322524 | GQTST | 356047 | QEGAD | 377486 | ASKRS |
| 265960 | KSGQQ | 322527 | QAQVS | 356054 | RNPGP | 377492 | QESWK |
| 265963 | LMKKT | 322530 | ASPTH | 356056 | FGLSI | 377494 | PRVSV |
| 265965 | RNGGL | 322532 | QGPLD | 356058 | INVPA | 377496 | EPTPM |
| 265968 | VEKKE | 322542 | PDPTW | 356062 | KFYKL | 377499 | GAHGF |
| 265969 | RGMSI | 322544 | SRVTA | 356063 | MEETE | 377502 | THFLL |
| 265970 | AATYL | 322545 | EENLW | 356064 | TQRLK | 377504 | VFSQL |
| 265978 | GPLNP | 322546 | CFWKD | 356066 | MKIRN | 377533 | KEKRR |
| 265981 | QPHRP | 322568 | LVFTY | 356067 | SLTQK | 377537 | TSPES |
| 265983 | LGCTH | 322569 | KCLIR | 356070 | AALAH | 377545 | DPNEV |
| 265986 | MAAKL | 322579 | KSGEP | 356071 | KDLEN | 377547 | GCFDR |
| 265990 | MHSLK | 322582 | STHRR | 356076 | PKLIS | 377551 | SKRLA |
| 265992 | PCFER | 322591 | ACCLH | 356086 | SPITR | 377558 | PGGRF |
| 265993 | NLETM | 322593 | HAFCY | 356087 | APPDV | 377566 | SIAEP |
| 265997 | PFRWS | 322594 | KECFC | 356093 | DQVAM | 377567 | NSFVR |
| 266000 | LSDSD | 322608 | VDMEI | 356094 | ETIIF | 377571 | SRLIS |
| 266003 | QHAAK | 322609 | NNVQV | 356099 | GSGRR | 377572 | NTFGY |
| 266014 | ISNIK | 322617 | VLWNT | 356104 | HLHKL | 377576 | QPSCC |
| 266022 | YKELD | 322628 | GKKRR | 356105 | TDVGD | 377577 | DTGTF |
| 266025 | APPTL | 322629 | VIAYN | 356110 | PSGED | 377582 | ASSCC |
| 266031 | RKSMW | 322640 | RCLAS | 356111 | SNPEP | 377583 | RTSSC |
| 266037 | GRGEQ | 322649 | TPWRC | 356113 | AKYSA | 377585 | CEPRC |
| 266041 | WSVEL | 322652 | DAEED | 356115 | ECYQR | 377588 | ISSKI |
| 266058 | CALCA | 322693 | DVPLQ | 356116 | QIGQL | 377596 | TAELP |
| 266066 | AAEPH | 322697 | ASQMS | 356121 | KSLFG | 377603 | EIPAK |
| 266068 | DHERK | 322706 | SNGEH | 356123 | CVSDS | 377604 | EIPAK |
| 266069 | IEEPK | 322716 | RAYIH | 356125 | CSKLT | 377612 | DKGSS |
| 266070 | TASQA | 322724 | KTKQI | 356130 | LDDST | 377617 | GVATN |
| 266077 | QRFLD | 322727 | VSWVY | 356133 | LKDYV | 377626 | IKFGH |
| 266079 | IKNTF | 322730 | ICGVV | 356134 | ENYRL | 377640 | VGGKR |
| 266081 | STFVA | 322754 | VGGAQ | 356135 | GGLQQ | 377653 | YQSSK |
| 266082 | PSSKC | 322775 | DQVLH | 356140 | IYSLA | 377655 | KGSSS |
| 266085 | NATDP | 322784 | GKKLF | 356142 | SHGKS | 377656 | PLPSA |
| 266086 | HGYYA | 322788 | NCRKN | 356143 | LSQNS | 377657 | RGQHT |
| 266088 | HAYFA | 322791 | AQMRV | 356146 | ETEGT | 377665 | IDDRK |
| 266116 | GFGKS | 322801 | FTYDK | 356150 | KVFIA | 377672 | GQHGH |
| 266119 | METSP | 322802 | VSQSN | 356155 | SHGTL | 377678 | VKPFS |
| 266123 | ATSIL | 322804 | APRFS | 356156 | VLKQC | 377696 | EMKKP |
| 266126 | DDHVL | 322807 | PESLR | 356159 | YTMRV | 377702 | FAEVT |
| 266157 | LSSLP | 322823 | NSHNF | 356169 | LHHRL | 377716 | NPPPA |
| 266182 | GGKGN | 322824 | NKEGK | 356175 | DRGSW | 377727 | SVGKE |
| 266214 | GAPTL | 322832 | SYRPF | 356177 | QSVSN | 377744 | IKILY |
| 266254 | LTTWK | 322837 | FRKTD | 356180 | EMIIS | 377746 | PNTYH |
| 266263 | KPSSS | 322851 | RVQTP | 356182 | KLLTH | 377747 | NVRKT |
| 266269 | GPEGK | 322858 | ASVEV | 356186 | LLPDE | 377751 | KSKRK |
| 266304 | KYGPL | 322866 | DKGAG | 356188 | LETSV | 377778 | PCVIL |
| 266383 | QMKTL | 322872 | EGSLL | 356191 | IGRFT | 377783 | KTKNS |
| 266395 | QFGII | 322883 | DDSFS | 356198 | CCTWN | 377786 | GFAVE |
| 266397 | PKVEL | 322885 | CLEQQ | 356202 | ISQKV | 377788 | GCRLY |
| 266427 | QEDEC | 322887 | ATHNE | 356207 | SAFRD | 377793 | TNIQS |
| 266432 | SSVSQ | 322898 | IVPPM | 356211 | FSLRA | 377796 | GKAVK |
| 266458 | SVYGK | 322899 | NLARH | 356213 | EELEK | 377799 | SVKKI |
| 266482 | KKSEC | 322915 | VELSD | 356214 | AFPDS | 377804 | VYVLW |
| 266483 | QRFMW | 322924 | VRCRC | 356218 | MRFTY | 377813 | SEPGT |
| 266497 | GNSII | 322939 | KEIRS | 356224 | GPPPL | 377814 | RGLTI |
| 266503 | IQWTL | 322944 | KFCKA | 356230 | VLREK | 377823 | MEVSC |
| 266505 | VWYVR | 322946 | GSVNK | 356231 | RVSHE | 377831 | QLNGN |
| 266508 | KRLKR | 322956 | ENCQL | 356236 | LGKNK | 377836 | FFLVH |
| 266517 | LKVPE | 322957 | QYRHH | 356239 | DTQEL | 377858 | IGGGP |
| 266524 | CLAVV | 322991 | WMFPV | 356243 | FHPDV | 377862 | AEQYV |
| 266529 | EELSD | 323012 | PALLS | 356247 | FASHV | 377878 | ILFAM |
| 266534 | STPTN | 323034 | ASPHF | 356251 | LGTPC | 377904 | LPDLL |
| 266542 | MNGKN | 323036 | SYGNI | 356256 | YRQYQ | 377927 | PVCFK |
| 266544 | KTVNI | 323046 | RLAES | 356257 | HPIPG | 377934 | RKNPL |
| 266546 | PPHRY | 323047 | AETLP | 356259 | LQLLH | 377936 | QDKVN |
| 266551 | DQALF | 323055 | PSLAA | 356263 | YDGPL | 377941 | GESDL |
| 266556 | VSQPS | 323063 | SAANF | 356265 | LQATL | 377954 | IYLVH |
| 266557 | PACSP | 323065 | GAPSS | 356272 | GRVFF | 377958 | VVNTR |
| 266560 | FRKVR | 323068 | KLVKS | 356278 | GTLVS | 377963 | ENTAF |
| 266579 | NSKHH | 323071 | AVPKL | 356290 | VKGLF | 377964 | NHYRR |
| 266580 | SSGVP | 323075 | LPDSY | 356309 | LKPTE | 377980 | QDHPE |
| 266581 | FVAST | 323076 | GQAAQ | 356311 | IISQV | 377995 | ICEWQ |
| 266594 | NLNNY | 323077 | QGLWE | 356319 | RIQWV | 378009 | CSCCD |
| 266604 | KGLAW | 323087 | EYVPR | 356320 | ILPGI | 378029 | SAWQL |
| 266643 | RVTTV | 323096 | GPMTT | 356324 | LFPHK | 378041 | RNRPY |
| 266646 | ACGCS | 323099 | EVECG | 356329 | QVSIS | 378072 | SSKVQ |
| 266659 | LVLLD | 323147 | PRRRR | 356331 | HAKRA | 378089 | SGCKS |
| 266671 | TSNSA | 323148 | LKSCV | 356351 | YRATN | 378112 | GTEEV |
| 266673 | FWPRG | 323155 | CSPKA | 356354 | SRDEL | 378118 | QNLKP |
| 266674 | FVPCL | 323172 | EFSIF | 356355 | CTASS | 378127 | RGRGH |
| 266679 | EYRHR | 323178 | SNPKM | 356363 | SYFYV | 378132 | GMSDI |
| 266682 | PESDL | 323183 | TVLLD | 356366 | YPFSQ | 378137 | CVMSV |
| 266688 | YGLVA | 323184 | LRLCR | 356370 | MERLI | 378145 | LNEKN |
| 266712 | ILNGE | 323191 | HMRKR | 356374 | RHLFS | 378161 | FSIRY |
| 266718 | EVTLN | 323194 | MSIES | 356375 | NEHHS | 378164 | TALQG |
| 266719 | QAVII | 323199 | DKKKD | 356379 | LGIPY | 378172 | SQGLP |
| 266732 | RGNKH | 323217 | PRRRR | 356382 | EPLRT | 378174 | LRPYK |
| 266735 | GEMRE | 323224 | KSYSM | 356384 | YPICE | 378181 | GPGGS |
| 266736 | LIYKT | 323241 | AAFEA | 356385 | SCEEK | 378190 | PPFSE |
| 266742 | LRAHF | 323246 | SGGAD | 356386 | YPRCE | 378191 | NSGIK |
| 266743 | QSMLF | 323252 | KDDAS | 356389 | DKTCI | 378201 | HLKRT |
| 266744 | FTNWF | 323264 | VQGYS | 356395 | YPRCE | 378203 | RSPNI |
| 266746 | LQQVA | 323275 | TELKS | 356399 | TCAKR | 378210 | AIQDG |
| 266754 | KKPRK | 323280 | DISAA | 356404 | HRKLH | 378213 | LLSTA |
| 266813 | KPPWK | 323288 | DWQKD | 356405 | SHLRF | 378244 | AAPLQ |
| 266839 | ESEGL | 323300 | LEFLS | 356410 | RKEFQ | 378248 | FRWFF |
| 266880 | TRFKN | 323302 | VQQKS | 356411 | TLDMI | 378260 | VPQGM |
| 266919 | SMSTN | 323304 | DSEQN | 356421 | AQETK | 378261 | QLPES |
| 266970 | PHLRL | 323313 | KKEEL | 356429 | ANSLK | 378262 | GRSSR |
| 266971 | VYVSP | 323327 | ESCLA | 356430 | VAIWL | 378279 | QKKET |
| 266980 | VTTEG | 323328 | IKIPK | 356431 | LKIQK | 378288 | FPTSP |
| 266987 | MAGSK | 323338 | LPVIP | 356432 | TKLCS | 378289 | GAGHQ |
| 266988 | PAKAL | 323339 | ITGSY | 356433 | DIYKR | 378293 | SETAL |
| 266991 | EELLG | 323348 | RHRRY | 356434 | HNISI | 378295 | SLKKH |
| 267008 | PKVIQ | 323354 | KIYFS | 356435 | PSILQ | 378296 | GALAE |
| 267012 | ENPQL | 323355 | VLVVK | 356436 | SKPKA | 378298 | RLNAV |
| 267015 | FHRLH | 323362 | EHPAD | 356437 | GPEAM | 378306 | NHTAQ |
| 267017 | RFGKK | 323374 | TDGGC | 356438 | RSTEL | 378311 | KNNSS |
| 267023 | RSSKR | 323377 | AKLLK | 356444 | SSQSR | 378312 | GLQEK |
| 267064 | VPPPQ | 323387 | GPRKS | 356448 | RGNIN | 378313 | FPQEA |
| 267079 | ASLPP | 323421 | NPNEQ | 356452 | WYNCP | 378317 | AIVGQ |
| 267082 | DSPTL | 323423 | KLLNL | 356465 | KSQCC | 378320 | IPREL |
| 267101 | NAQRT | 323424 | NPPTD | 356468 | KIFQF | 378323 | SSNIQ |
| 267102 | KTQHQ | 323427 | PPGFK | 356470 | LNYRI | 378324 | QCQTA |
| 267103 | LPQIS | 323433 | DQVCT | 356473 | TEELE | 378329 | VSASE |
| 267115 | KKEKK | 323435 | LPPMP | 356476 | GCRRW | 378332 | LKLFG |
| 267116 | GCYSE | 323439 | DDDQR | 356480 | TKEHK | 378338 | REKKQ |
| 267119 | KKTSR | 323447 | AGRRE | 356481 | LPSEE | 378345 | PQDNV |
| 267122 | GQFSL | 323455 | LLLDL | 356482 | EKDEL | 378346 | TANYF |
| 267129 | VPEAG | 323479 | TTCHY | 356492 | SRDEL | 378349 | YHDEL |
| 267163 | NKEEK | 323500 | LPPTT | 356509 | RKKGK | 378350 | SSVTP |
| 267169 | YLRED | 323507 | VTVQE | 356515 | RPQAM | 378357 | LDVWS |
| 267176 | NLSNA | 323511 | QFSEG | 356520 | GGGGY | 378359 | CILLL |
| 267197 | RGTLN | 323516 | PRAPA | 356529 | EPSHT | 378364 | RGYVK |
| 267199 | MEKPF | 323527 | DLITL | 356530 | ASPGC | 378366 | TGVQV |
| 267202 | GFILQ | 323549 | DTDMR | 356531 | IKKEK | 378396 | KIGII |
| 267205 | LCLLL | 323555 | CNIFV | 356532 | GRTLG | 378400 | DEDEE |
| 267260 | SSTRR | 323557 | ASAKT | 356534 | HYCMD | 378407 | GPTAE |
| 267273 | ILKWD | 323568 | ATETV | 356536 | PKSSP | 378414 | GIRNT |
| 267291 | EGKKR | 323571 | PIRRH | 356540 | CPCLK | 378426 | KAKRH |
| 267294 | VRTIH | 323580 | DLLPE | 356541 | KPEEN | 378430 | QAGES |
| 267328 | SGCCA | 323584 | ELEKQ | 356548 | EGLKL | 378446 | LDQVN |
| 267339 | LHHGS | 323586 | NRKRQ | 356549 | AIVAF | 378461 | KFSPY |
| 267377 | RITTL | 323587 | LLFYR | 356552 | PTPDR | 378463 | RGTKF |
| 267383 | EPPAP | 323588 | PLSHM | 356560 | FLVFG | 378468 | RGIQV |
| 267396 | DLSVL | 323595 | RQDSR | 356562 | DEANT | 378474 | GPLNY |
| 267406 | LIFPL | 323606 | FLSKK | 356563 | QQLEK | 378476 | FEVKS |
| 267407 | SQLLT | 323612 | FINKL | 356567 | TTGKA | 378479 | NAHSS |
| 267415 | SSRPL | 323633 | VAYNM | 356571 | PQLTP | 378491 | VTSQL |
| 267422 | PQKVG | 323635 | KKKVE | 356574 | GHPAA | 378495 | HLSQR |
| 267425 | SILED | 323645 | YSLKI | 356579 | RMLLV | 378510 | NTADH |
| 267426 | SRKHN | 323663 | AVDSG | 356581 | HSTKF | 378515 | TSWSL |
| 267430 | LKSDI | 323670 | TAGLA | 356587 | DSPML | 378516 | NCLSQ |
| 267434 | ATDKS | 323678 | VNSKL | 356590 | PKKGH | 378517 | SSEVN |
| 267436 | QRFEL | 323680 | FSSPQ | 356591 | CRYVF | 378524 | NGLIK |
| 267460 | QGPID | 323686 | RSNPF | 356595 | DVVQR | 378529 | FTRIR |
| 267483 | LSKSN | 323687 | CTEDK | 356599 | NYFLS | 378533 | DSLLQ |
| 267484 | KRHAE | 323688 | FHTKK | 356602 | TTGRE | 378534 | ESDGD |
| 267485 | SEDEF | 323694 | SCTLA | 356607 | LMTFE | 378546 | FEHAT |
| 267499 | AHSSG | 323696 | LEETS | 356608 | RTLEG | 378565 | VEGEA |
| 267502 | GIRWE | 323697 | VLTFS | 356620 | FPFRQ | 378578 | LTKTP |
| 267512 | SHSRV | 323714 | NKFVV | 356623 | SRVSC | 378599 | GEKKP |
| 267522 | RRKKQ | 323720 | PGGRP | 356630 | VLEAY | 378617 | RHYTS |
| 267540 | TMFAW | 323732 | SLKLM | 356632 | ILLQD | 378619 | QGYLC |
| 267549 | LEVLE | 323740 | RESPF | 356634 | AEENQ | 378621 | SLASF |
| 267550 | AIFTQ | 323765 | VGDAK | 356635 | PFSHL | 378624 | GFSLI |
| 267568 | EEISL | 323777 | QPEDV | 356641 | VLELV | 378629 | TKVFS |
| 267569 | QLEKK | 323780 | RDENQ | 356642 | TLESH | 378630 | SESTH |
| 267584 | NPEAQ | 323782 | GERKR | 356647 | TPSIP | 378635 | TLLAS |
| 267594 | VFGYL | 323795 | QTEGR | 356650 | QRATW | 378648 | FEECG |
| 267620 | SQYPF | 323807 | GNGDG | 356652 | GDTEF | 378649 | QTRPH |
| 267622 | DLLKQ | 323811 | TRSNM | 356655 | SSMEK | 378653 | KQQLA |
| 267750 | GGLLL | 323816 | CTFLP | 356658 | NSCSI | 378657 | RRESF |
| 267803 | HTPLI | 323821 | RHTQK | 356671 | NPCVK | 378669 | SNHAY |
| 267812 | KRKTT | 323822 | TLQHN | 356674 | TTGPD | 378680 | AHPGD |
| 267814 | SDQNP | 323827 | KRSFL | 356678 | HPQII | 378685 | PTRKE |
| 267837 | HYENF | 323830 | YNHFC | 356685 | DDTSN | 378699 | QIKKM |
| 267838 | SLYHS | 323834 | DLCCP | 356687 | RQKMS | 378702 | DSDSG |
| 267842 | KTLKL | 323837 | DKYGY | 356689 | LAWTL | 378718 | CLKQH |
| 267843 | PMAIT | 323846 | VLKET | 356694 | GLYKL | 378733 | ENKEQ |
| 267845 | LQAMV | 323847 | FFKQN | 356696 | VSGHI | 378741 | QAQQA |
| 267857 | YGTEL | 323856 | ESAVA | 356700 | KTTRQ | 378750 | HSPGF |
| 267859 | PKNEQ | 323879 | EHPSD | 356708 | KDLLK | 378758 | RPVSS |
| 267869 | SNTTE | 323880 | VGAFL | 356713 | SKEFS | 378774 | MGEKS |
| 267884 | ATAAQ | 323881 | NAVDY | 356723 | SLWRG | 378782 | VPKIH |
| 267889 | SGYSS | 323889 | ICSPK | 356729 | FLVLT | 378784 | TLCSV |
| 267918 | GEDDD | 323890 | ALSLP | 356732 | QDLRS | 378792 | PQEVF |
| 267935 | MSSLG | 323895 | HVPES | 356734 | VPTVN | 378796 | FRSEL |
| 267938 | PKEDG | 323901 | GETTV | 356737 | AALAT | 378798 | KEALM |
| 267950 | ILKKK | 323913 | NSLNE | 356738 | WMFAL | 378802 | RFVHT |
| 267953 | LKQYC | 323926 | ANKIK | 356744 | EDNNW | 378812 | ANGLA |
| 267970 | GGTYA | 323927 | EAEQC | 356761 | EGDSP | 378813 | PAGHM |
| 267973 | YDCPI | 323929 | VLGNF | 356770 | GCDIY | 378817 | AEEDY |
| 267974 | TRYYC | 323945 | ECEGS | 356775 | VTLIE | 378820 | QSTSL |
| 267978 | QPPPH | 323952 | KAIYE | 356779 | TDHYN | 378836 | SPKNC |
| 267984 | SNSVN | 323967 | DEKKE | 356785 | KILDN | 378837 | HLQPR |
| 268035 | QSSTC | 323978 | HLEIT | 356789 | IEVKS | 378845 | LQELN |
| 268042 | VSFIL | 323982 | RQDSR | 356791 | EFANV | 378848 | PGRDY |
| 268053 | EATQQ | 323986 | LPILH | 356792 | VTLTG | 378851 | PHSYH |
| 268057 | QIREK | 324000 | KTGCF | 356793 | VTLTG | 378856 | THVQT |
| 268058 | ASQQS | 324020 | KRSES | 356795 | SSLDG | 378857 | YNWYD |
| 268070 | PPPNS | 324025 | GKRKR | 356807 | STQVE | 378865 | KHLAC |
| 268074 | PCFSP | 324056 | ATGLE | 356811 | TEIKE | 378879 | NYLEG |
| 268079 | VMQDT | 324060 | ILRIR | 356814 | NEDEE | 378885 | EESGE |
| 268097 | EFEQT | 324064 | KAYEV | 356825 | VDNHF | 378906 | IIVGG |
| 268099 | AFQGN | 324074 | EEEDD | 356830 | QDTKM | 378912 | VPLLV |
| 268122 | VPLVP | 324088 | GAGHT | 356832 | TDSFL | 378936 | PQFHY |
| 268124 | SQPGP | 324101 | KLEHY | 356834 | APEEG | 378937 | LLGRV |
| 268129 | EADLE | 324103 | PVDCK | 356836 | ASKAQ | 378951 | CQPPE |
| 268130 | ENFKK | 324105 | NPKAK | 356842 | DHLDQ | 378957 | KIRRA |
| 268134 | PSTAH | 324111 | RIVNI | 356846 | FSSLS | 378958 | ASPHS |
| 268138 | WLEDL | 324122 | GLMST | 356848 | GLLLC | 378969 | QGKSM |
| 268148 | RHCCT | 324124 | APLEK | 356849 | MAESN | 378985 | NPFLL |
| 268150 | ELLGC | 324127 | KQKVT | 356853 | SSRPH | 378986 | PDPHA |
| 268154 | YYNVL | 324173 | EKDEL | 356856 | SGKFS | 379003 | RGARR |
| 268159 | NQDGE | 324175 | LEDPW | 356858 | PKCCH | 379008 | YAYFA |
| 268164 | CDGAT | 324191 | SQIKK | 356873 | GNMYF | 379028 | MNLNL |
| 268171 | DQSAL | 324196 | ESKEA | 356879 | QELIK | 379029 | DIASN |
| 268182 | KFYGK | 324203 | VGELI | 356885 | VPQCA | 379034 | LQTSL |
| 268184 | RADRL | 324204 | GVSQQ | 356898 | QQGDE | 379035 | ATPGE |
| 268206 | LSKNK | 324241 | VYYIN | 356905 | CSLSW | 379038 | LPPQQ |
| 268220 | LVHRE | 324243 | IPVTS | 356906 | VDVLP | 379045 | TTRKL |
| 268251 | LADFK | 324248 | GFMRF | 356908 | QKESE | 379047 | LFKAS |
| 268261 | ELLFS | 324251 | TISLL | 356909 | PVLSS | 379048 | FLYKI |
| 268271 | ESPEK | 324270 | QPSTA | 356912 | AYLCL | 379059 | PEGLL |
| 268281 | LLIQS | 324277 | AGLFF | 356919 | PESLQ | 379065 | GKPKE |
| 268297 | NVPLS | 324301 | LASCL | 356920 | QTGKI | 379074 | LGHWV |
| 268314 | ARVPA | 324302 | MKHKC | 356925 | TPVES | 379078 | PPVNT |
| 268333 | SRSSS | 324304 | DTYCA | 356937 | DQNRI | 379114 | GNKEN |
| 268349 | LEAKP | 324318 | CLKLA | 356940 | STKSL | 379121 | RAENS |
| 268379 | FVDEL | 324323 | GCFYY | 356941 | VKTNI | 379122 | PGTWK |
| 268383 | LSSHS | 324331 | FLFFF | 356946 | DPQDK | 379140 | SPGDV |
| 268389 | NYERP | 324343 | TIVKE | 356951 | RPAKS | 379145 | FLHWL |
| 268459 | HFYQT | 324352 | PGSSR | 356953 | GLAAM | 379146 | GGESM |
| 268482 | ARFGL | 324369 | VIRKG | 356954 | YGDMA | 379158 | KPKDD |
| 268483 | LYQDI | 324392 | NGAEI | 356959 | QEICS | 379168 | AKHRN |
| 268489 | DTFRL | 324403 | PQGHN | 356966 | SITVG | 379173 | HKEKN |
| 268509 | MASLD | 324404 | KIAWN | 356969 | QPATQ | 379174 | SLITV |
| 268533 | ATSRL | 324414 | TMKNN | 356971 | TTMPS | 379183 | STVEF |
| 268595 | AKGKK | 324419 | LVEKR | 356972 | GEVDR | 379198 | SDSDD |
| 268603 | FDDDS | 324423 | NWRAD | 356975 | WAGRK | 379201 | SKFPS |
| 268605 | ESEDS | 324435 | SPFYR | 356982 | EKCNQ | 379203 | LLEFK |
| 268607 | MKLSV | 324444 | THHLP | 356984 | LQALP | 379209 | DSRRH |
| 268613 | SLACL | 324445 | SRALT | 356988 | GHPLS | 379223 | PAPTQ |
| 268616 | LDSTD | 324450 | GGKAS | 356989 | GVLVS | 379227 | TFGLE |
| 268624 | GKFRN | 324458 | LKDKA | 356990 | ILDFI | 379253 | DRPRL |
| 268638 | QQITV | 324463 | RTKYI | 356991 | RGHLV | 379259 | PTVRY |
| 268655 | LHHGD | 324464 | SVVHR | 356992 | KGEGL | 379265 | CCSIM |
| 268661 | TSGDL | 324468 | KKVFL | 356999 | KNDSN | 379279 | LLHYN |
| 268668 | AAATS | 324469 | RRWGQ | 357004 | LEKES | 379309 | IKRGR |
| 268674 | SGSNH | 324491 | TLKPL | 357008 | LLFYR | 379313 | LLSYS |
| 268676 | PDVEA | 324510 | MEADP | 357012 | FDVYS | 379326 | ETTHF |
| 268679 | DTVPR | 324511 | QSDAL | 357022 | YENVI | 379330 | ARNQT |
| 268695 | CLWSH | 324517 | PTSIS | 357025 | GLLLT | 379336 | HHEWP |
| 268699 | VGTPT | 324527 | SVPGN | 357036 | LDNVV | 379339 | HCLWR |
| 268704 | PTWPK | 324532 | GCPDQ | 357037 | LPVTQ | 379345 | GEVTT |
| 268711 | EIPKE | 324534 | YSENV | 357040 | SETSV | 379363 | LHAGE |
| 268717 | PSSYS | 324549 | SLASS | 357048 | PLQFR | 379368 | THLVE |
| 268719 | IYEFR | 324551 | GCTIV | 357051 | QCLIM | 379378 | LSAAT |
| 268720 | PGCTP | 324553 | KPTLV | 357054 | PPKKA | 379379 | PIGKP |
| 268766 | EPVPP | 324557 | GKSGD | 357057 | DDDDD | 379397 | PILMT |
| 268793 | TQWLC | 324558 | RSCAP | 357060 | RETYY | 379399 | KEKAT |
| 268794 | GGEDD | 324560 | TGICA | 357067 | SESKV | 379401 | SLREP |
| 268795 | LILWL | 324570 | SPARG | 357068 | RISNV | 379406 | HSASA |
| 268797 | QRNNL | 324573 | CKALA | 357069 | RIKYD | 379430 | WRKTY |
| 268802 | FVKKR | 324580 | RWPQG | 357072 | GSSPA | 379435 | LKHQL |
| 268835 | IGLDD | 324583 | AHRSR | 357076 | PRQIL | 379438 | TLTFQ |
| 268854 | LEDKS | 324587 | TFAAH | 357079 | ATSVN | 379441 | TDWEN |
| 268864 | RCAIM | 324595 | PEGIV | 357080 | NGLLV | 379450 | FIFVK |
| 268876 | IKPVS | 324598 | THNVI | 357086 | EEVLC | 379458 | MRPYK |
| 268896 | YLKKT | 324605 | EKETS | 357087 | YQRTK | 379464 | TAGRL |
| 268919 | KSKVE | 324617 | VEKWR | 357097 | NPKNN | 379500 | QQLKL |
| 268933 | TNPFL | 324628 | SGGLC | 357103 | LGSKS | 379503 | YQNQE |
| 268957 | PVMAN | 324633 | CRRKK | 357105 | ENNRK | 379504 | LSHSL |
| 268981 | VMNET | 324636 | LPMVP | 357106 | KEYFI | 379507 | ATGCK |
| 268989 | LIENK | 324648 | RFLPR | 357111 | SSQLV | 379520 | ETTPR |
| 269025 | KRSKK | 324651 | QVSEI | 357123 | PMNVN | 379528 | SPGSQ |
| 269033 | FYNTM | 324687 | AHRSR | 357125 | SKLLV | 379537 | KFYGK |
| 269051 | LPPPP | 324693 | TLTEI | 357126 | GWAGK | 379553 | FCKGT |
| 269053 | DGCDF | 324694 | KPQKE | 357128 | NKPGG | 379566 | PQMSS |
| 269064 | KSPSL | 324701 | SLDKL | 357130 | SYFGN | 379568 | RRKLN |
| 269080 | PQEEP | 324709 | WSGSC | 357132 | VALKK | 379571 | SRLEQ |
| 269081 | PQEDP | 324729 | HGKNF | 357133 | SCPAN | 379583 | DTFWD |
| 269097 | PIHSS | 324735 | QILDG | 357134 | LKLYN | 379584 | PAAGI |
| 269122 | FGYSM | 324740 | PGENL | 357135 | CLNSK | 379590 | LKTHS |
| 269138 | GNYQQ | 324741 | VEPVK | 357149 | LNQLW | 379599 | VYFST |
| 269141 | GGGDD | 324763 | NRFQD | 357150 | YQNIP | 379605 | GTLAG |
| 269142 | LALLK | 324767 | LPLDI | 357151 | IINIC | 379612 | VLNFD |
| 269143 | EKVAN | 324769 | ALAGK | 357153 | YQGVL | 379614 | TLAIH |
| 269159 | RDDEK | 324774 | STEGT | 357155 | MQTHV | 379616 | TKASE |
| 269187 | FRINF | 324775 | KKGEK | 357156 | VICSG | 379627 | IYEKV |
| 269190 | VSLQG | 324788 | HGEAA | 357158 | YEDAM | 379629 | LGTLK |
| 269195 | LPEIF | 324792 | IEVWG | 357169 | CLCNT | 379638 | IYVDI |
| 269197 | KEQLK | 324799 | PTSAS | 357175 | AAADA | 379646 | SSSSC |
| 269200 | RIAYL | 324803 | GKSGE | 357177 | ASPGP | 379651 | DAAGR |
| 269201 | PLISR | 324804 | VLSLA | 357178 | GGPGH | 379654 | PITKL |
| 269202 | NQHAF | 324806 | ASNST | 357189 | DHESL | 379669 | GYPKK |
| 269214 | GQNST | 324810 | PPSVS | 357196 | YLESS | 379678 | PTVIT |
| 269216 | ALALA | 324820 | QTKEH | 357199 | ETEDS | 379680 | EMSQQ |
| 269218 | SIPLA | 324827 | STWNR | 357204 | VYVRE | 379684 | GKAFN |
| 269221 | RPTTF | 324831 | RNQLF | 357206 | SSGED | 379688 | FILIS |
| 269228 | RLLNF | 324842 | ELQGF | 357212 | QKKRD | 379689 | EKLHV |
| 269280 | LPLSS | 324856 | ACKQQ | 357218 | VYRLQ | 379693 | NLVSL |
| 269289 | ARRRK | 324857 | NKYTY | 357220 | ICTIV | 379694 | DSDSD |
| 269290 | PGCPR | 324870 | HPGKC | 357225 | GLDSL | 379699 | ACYSL |
| 269299 | EPPLL | 324876 | GNTWN | 357229 | GAWRA | 379701 | KQKQL |
| 269300 | SGALP | 324878 | KLFIT | 357231 | EKHQK | 379704 | CPTAE |
| 269305 | GPDSD | 324882 | QRPPH | 357234 | PQFSK | 379707 | SHILT |
| 269321 | KDWKD | 324884 | KLTHL | 357244 | DAGQE | 379712 | DEWRE |
| 269346 | NQFPA | 324890 | AAYRS | 357251 | LFFVL | 379713 | FGRRD |
| 269347 | SCKIS | 324892 | DMFWS | 357259 | QEGGV | 379732 | KGRRG |
| 269349 | VADLI | 324895 | CCKKP | 357276 | GKFNK | 379733 | IRRMN |
| 269373 | RNLVK | 324897 | KFAPS | 357283 | NCSIM | 379738 | PVTQL |
| 269383 | EEVLS | 324901 | KIPSS | 357285 | VRKEK | 379744 | QRTRG |
| 269384 | IFHSE | 324913 | TDDKR | 357292 | GSQLS | 379746 | TAQNT |
| 269385 | FKEKR | 324920 | QFTFF | 357295 | KTKKN | 379747 | TSGWG |
| 269389 | YAADP | 324944 | KRKTD | 357297 | TVTEV | 379748 | YYPQQ |
| 269391 | GPLAV | 324948 | AVSAN | 357298 | VASES | 379751 | PLQML |
| 269392 | TPSTK | 324956 | DEVED | 357300 | RRRYL | 379754 | SSIIF |
| 269394 | RARVS | 324958 | ENIKL | 357301 | RGTPG | 379757 | TRKKQ |
| 269397 | EYVTV | 324960 | MLSRH | 357304 | FWMAP | 379760 | VPQST |
| 269439 | LGADS | 324967 | RGSGG | 357305 | KDSVT | 379769 | KELQQ |
| 269445 | TMDSD | 324984 | NAHLH | 357307 | SLSEY | 379770 | GVPGK |
| 269466 | IPIKK | 325002 | LASVG | 357308 | KKSRG | 379785 | LNGDT |
| 269468 | GARKQ | 325012 | NIYHV | 357315 | EELDE | 379787 | QWGTK |
| 269485 | GGAKA | 325016 | GLYPS | 357324 | ISGFL | 379791 | ALTED |
| 269499 | LDSAD | 325017 | RRHRS | 357329 | SFPAG | 379793 | PAGEE |
| 269500 | FGTEY | 325018 | RIHTH | 357330 | NSFRK | 379799 | LASNS |
| 269503 | LVFPL | 325027 | TPIRI | 357336 | TSLVL | 379813 | CTTFI |
| 269518 | RLHCN | 325032 | GIIDS | 357341 | VWAFD | 379824 | RNGLE |
| 269534 | EMQGP | 325038 | EKPYK | 357342 | LSANM | 379832 | QQSKP |
| 269554 | SNILT | 325040 | VSLLM | 357345 | RDISR | 379836 | GTYIM |
| 269571 | LDVPV | 325041 | AARPH | 357348 | EVGAF | 379847 | PGLDD |
| 269576 | KGPRY | 325065 | WLYHS | 357354 | RETGL | 379852 | RTHAD |
| 269577 | EDDLF | 325078 | CLFLC | 357356 | QGLQA | 379855 | AQQRR |
| 269582 | QKVGL | 325091 | KNEDL | 357360 | EDEEE | 379865 | SMKLY |
| 269586 | LVAAL | 325120 | LFHKK | 357362 | LQGRV | 379877 | FGLPQ |
| 269593 | DSFRE | 325123 | KEKLY | 357384 | RGLRF | 379889 | KHSMS |
| 269601 | TKYRY | 325128 | ATNRR | 357387 | EELQC | 379895 | QSYLD |
| 269701 | AVPTE | 325136 | EAYPK | 357389 | WLLQT | 379903 | ASLST |
| 269703 | LPPRA | 325137 | LKHRI | 357392 | LLQTP | 379925 | GGGCC |
| 269720 | EERKP | 325140 | RFSSP | 357393 | TFLAS | 379927 | SDDEF |
| 269724 | DGFLG | 325144 | TQTRP | 357394 | LLRIL | 379932 | KITVW |
| 269740 | VFLKW | 325146 | YPGSG | 357402 | MYMSK | 379938 | EFSRK |
| 269829 | TGEKP | 325155 | HFMCV | 357409 | HPPPK | 379942 | ALAQG |
| 269834 | RIHSR | 325156 | RPRPH | 357412 | KELLD | 379948 | PSFSS |
| 269844 | QMYSY | 325161 | LAQRP | 357425 | IKHNY | 379960 | RWKSR |
| 269856 | FIELH | 325166 | ESRSG | 357431 | QNEEK | 379965 | KKKKK |
| 269878 | FHIRI | 325184 | FLKSW | 357432 | VERKL | 379967 | IANKK |
| 269881 | RRNEL | 325203 | TSQRK | 357440 | PLLDS | 379974 | VAQMG |
| 269886 | VPLPQ | 325209 | KVFHP | 357442 | AANDG | 379992 | VCPSC |
| 269919 | YCHKH | 325219 | WGGCH | 357451 | EIREE | 379994 | VCPSC |
| 269945 | ISLLS | 325239 | AKDQE | 357452 | IRSRL | 380009 | LYCPG |
| 269967 | ELDGD | 325240 | YVEAI | 357461 | APSSK | 380019 | HLASS |
| 269973 | HRKTR | 325255 | EHSYP | 357470 | YFFPR | 380024 | ERKKK |
| 269981 | FLNDT | 325285 | ATASS | 357475 | KPLKG | 380025 | LRSKS |
| 270001 | IHNGI | 325290 | HSLFK | 357477 | NCITQ | 380041 | AASEE |
| 270014 | FLNDT | 325296 | TLQRS | 357480 | QRLGK | 380053 | GISDA |
| 270059 | LLNPI | 325301 | PGSRT | 357484 | HKKSY | 380055 | LGSRL |
| 270061 | MTMSV | 325312 | RGDDY | 357494 | GDGSD | 380082 | FELEA |
| 270077 | TESQS | 325313 | CKVEF | 357501 | DEAKL | 380085 | SNHQP |
| 270112 | VKTQC | 325326 | TLADD | 357507 | LESTL | 380090 | KPSMY |
| 270115 | SPVPT | 325343 | SALKI | 357511 | YVERF | 380092 | GSSNP |
| 270139 | QQDFV | 325355 | DAMLD | 357513 | DMTSI | 380094 | WTRLL |
| 270142 | IGIAQ | 325369 | LPSFK | 357515 | GMFQL | 380108 | SCLSL |
| 270162 | FVLVQ | 325370 | VVTPC | 357525 | AAGGT | 380116 | EARRL |
| 270172 | LTSSL | 325376 | IDRNA | 357534 | DCLAA | 380117 | GLSNS |
| 270176 | ARKLD | 325377 | GGRGR | 357535 | NFKDQ | 380120 | DVENL |
| 270190 | EHPEC | 325381 | SLSPG | 357542 | IYHPQ | 380133 | KRDYV |
| 270202 | ASGTA | 325386 | SLLNF | 357545 | QAKAK | 380139 | KFWKR |
| 270221 | LRKRE | 325393 | LTECV | 357547 | WSIRH | 380143 | ACHIQ |
| 270223 | SGTVV | 325398 | TKDVL | 357554 | SAAQP | 380147 | KRGKQ |
| 270225 | CLGPK | 325402 | RWKSP | 357555 | RRKQH | 380148 | IYFFT |
| 270233 | FGDEC | 325414 | IILGS | 357556 | TGDAF | 380153 | AVGGK |
| 270235 | PRPEH | 325421 | GGGSK | 357561 | KATFQ | 380155 | HASEP |
| 270238 | GPVEN | 325423 | AETHT | 357564 | KCSLG | 380174 | QSAPK |
| 270257 | YTFKP | 325425 | QTVFH | 357565 | AEKIP | 380178 | ETAFE |
| 270279 | DPAPA | 325431 | RVRRG | 357567 | DGWFA | 380181 | HCTRP |
| 270281 | HWESW | 325438 | DVSSL | 357574 | HHSCF | 380182 | FFAFF |
| 270288 | HVTEL | 325448 | VGTSV | 357575 | KVKKL | 380223 | VCRCL |
| 270301 | KARGH | 325452 | GALAG | 357579 | SDSAD | 380229 | ASLKK |
| 270328 | TCHGH | 325458 | NQSEA | 357581 | AGDAL | 380243 | YKASD |
| 270349 | HWLKV | 325485 | YWAEE | 357583 | CCVVQ | 380248 | SSLVL |
| 270357 | VNVSA | 325506 | LLALD | 357585 | IELKP | 380251 | RKSHL |
| 270443 | KVFSR | 325508 | EILVI | 357588 | TAHNM | 380253 | DVKKN |
| 270451 | RWKHP | 325511 | NTSLL | 357591 | YRGKK | 380254 | HCAWP |
| 270457 | EKPYK | 325519 | CSMGG | 357594 | STPEL | 380262 | EDILF |
| 270458 | KTTPV | 325525 | LEGVF | 357597 | GFQSG | 380268 | NTFWI |
| 270459 | GGEGR | 325526 | RYVCK | 357598 | TQFET | 380273 | LNAEV |
| 270460 | NPFLL | 325527 | QVLLH | 357600 | RFQSG | 380278 | EPRRP |
| 270474 | GGDPT | 325546 | HQAFQ | 357615 | NNFIR | 380282 | AGHCT |
| 270502 | AEAPR | 325548 | SQLKD | 357624 | AEAAE | 380285 | PYKCK |
| 270509 | QLQLL | 325555 | IKKSE | 357625 | STSTF | 380290 | SQTFL |
| 270517 | QQGQS | 325561 | SGAEL | 357632 | LRKMR | 380293 | LTVHK |
| 270530 | QGLDN | 325562 | KEQEQ | 357643 | DSEDI | 380301 | KITLI |
| 270538 | TEQIL | 325564 | YCRCP | 357644 | ISGQK | 380304 | KNHTN |
| 270560 | QDTPH | 325589 | HVTAL | 357645 | EMHPK | 380308 | TRIPA |
| 270583 | SGAED | 325608 | QPLRW | 357651 | LSIAM | 380310 | TGENP |
| 270586 | TLPPA | 325612 | PEDRP | 357656 | PGENL | 380312 | TKHNS |
| 270590 | NAPRE | 325618 | RSPDT | 357657 | GEMGS | 380325 | ETIQM |
| 270593 | LGGPV | 325628 | RNRYH | 357660 | DGILF | 380328 | ITLQM |
| 270617 | QQYCL | 325634 | VHETT | 357662 | APPPG | 380329 | KWPEE |
| 270625 | QFQKF | 325638 | KRTPS | 357668 | TAYIK | 380331 | KICIP |
| 270631 | VLDAP | 325660 | VYLEF | 357669 | SSTRG | 380335 | MNEMK |
| 270632 | AVRRA | 325663 | RAPPY | 357674 | GSPGK | 380341 | GVTVR |
| 270634 | HGLLV | 325668 | ETEDV | 357681 | KSCVC | 380344 | FRRRR |
| 270637 | AVPTF | 325674 | LLLAE | 357687 | FWENS | 380351 | SNLTT |
| 270642 | LWWRM | 325675 | LVMDK | 357692 | QQSSS | 380352 | DQPLQ |
| 270645 | HHDEL | 325677 | RIYIS | 357696 | CPDRP | 380364 | CVAAL |
| 270685 | KETSF | 325681 | IFFLD | 357701 | PPCSQ | 380368 | GQAGL |
| 270691 | KLEEE | 325682 | WRLCG | 357708 | EALKG | 380369 | PSFFF |
| 270708 | LGGHT | 325683 | AFICV | 357711 | SGGSQ | 380370 | ATAAH |
| 270720 | SREFS | 325690 | MHYGS | 357713 | SGGSQ | 380371 | RRSLA |
| 270722 | PINHL | 325691 | SIYEV | 357715 | SKGYG | 380374 | ATMPG |
| 270747 | GEAPV | 325708 | LARYR | 357721 | ESHKE | 380376 | SYKFN |
| 270776 | SSYNA | 325713 | SEALP | 357726 | HTHKE | 380380 | LSGFD |
| 270800 | VQWES | 325727 | HCTLA | 357731 | TWPSK | 380383 | HNNNC |
| 270815 | PQDQA | 325738 | TRSSG | 357732 | PGKGK | 380388 | VIVTP |
| 270824 | RMHYY | 325747 | VAAVP | 357736 | CPPPE | 380389 | IPALW |
| 270861 | TPNFH | 325748 | LTSTR | 357739 | PPKSK | 380395 | YEKAQ |
| 270879 | RMMLR | 325775 | GPRDA | 357748 | GIALS | 380398 | AESAE |
| 271015 | TVSRQ | 325776 | SNLSS | 357753 | PPKHK | 380402 | NFCIF |
| 271064 | DMGHH | 325785 | KYFET | 357754 | SRPSK | 380403 | VSHCA |
| 271069 | KGPFG | 325794 | NIPCK | 357757 | SGGCC | 380413 | SPSLL |
| 271139 | ERICH | 325808 | AAGAL | 357759 | SGGCC | 380414 | KELIS |
| 271227 | QAIVR | 325817 | LATTC | 357762 | KSAYF | 380417 | PRVMG |
| 271263 | KLHLC | 325819 | SIQCS | 357768 | SGDCC | 380442 | ERAGA |
| 271277 | LPTSS | 325822 | FRAIN | 357769 | SGGCC | 380450 | PTTEQ |
| 271308 | VIKDI | 325827 | HLVLN | 357772 | SGGCC | 380451 | KKHDV |
| 271324 | QTIGL | 325836 | KCTLM | 357773 | SGGCC | 380452 | YFLMV |
| 271331 | KNINF | 325850 | GFQCG | 357775 | PHGCC | 380455 | CPGWS |
| 271332 | FNFLH | 325863 | RRNRR | 357776 | SGGCC | 380465 | RGVLA |
| 271348 | DDLSV | 325864 | LETIN | 357778 | SGGCC | 380476 | GGRWK |
| 271357 | AAEQR | 325868 | LVDVE | 357779 | CCSGC | 380480 | SLIIH |
| 271373 | AKAHK | 325875 | MEEVD | 357783 | HINST | 380485 | KSEER |
| 271375 | AVCLA | 325905 | PERMD | 357789 | YYYYE | 380492 | GTFNI |
| 271385 | KRRED | 325915 | VILHG | 357791 | CYFYQ | 380493 | FFGFF |
| 271417 | MSLVV | 325917 | EEQIC | 357794 | SQYRP | 380501 | PPHPA |
| 271450 | VNSNN | 325919 | PPALF | 357796 | STTKQ | 380504 | GQKHV |
| 271452 | FKMST | 325921 | DSLVD | 357798 | STLNM | 380506 | SNSSC |
| 271469 | VKNGI | 325929 | PGSLV | 357799 | QKGKK | 380507 | CCKWI |
| 271526 | AKYGF | 325937 | PSLTR | 357804 | AKSLT | 380514 | KMPRD |
| 271532 | KDEES | 325941 | DSDPD | 357807 | EESPQ | 380523 | TAWKL |
| 271555 | TWTLK | 325954 | PHPGK | 357810 | TAKLF | 380537 | LVAED |
| 271583 | RGICL | 325958 | VISKT | 357811 | SSLES | 380542 | KSKRS |
| 271588 | SAYPY | 325978 | HGGFQ | 357812 | DDYLL | 380547 | NGDTT |
| 271610 | LNQGS | 326003 | PALDD | 357820 | PSEPP | 380548 | PKASG |
| 271620 | SLSKK | 326010 | SLKRK | 357823 | TSPQI | 380559 | SDLSQ |
| 271628 | GPLPQ | 326018 | QAAAV | 357824 | DKKAA | 380572 | QSAGI |
| 271636 | QTSSC | 326021 | TFTPH | 357830 | PSLAF | 380579 | GPEGP |
| 271638 | SQKRT | 326022 | MDTDI | 357834 | RDVAT | 380580 | HTHRI |
| 271643 | PQDPS | 326030 | YKIHL | 357835 | QHADK | 380581 | ARGTQ |
| 271651 | SFPKM | 326031 | AKAKK | 357838 | TILSF | 380601 | HCTRP |
| 271657 | TNGIM | 326042 | EGEEY | 357840 | KGTNV | 380602 | ARSSL |
| 271699 | SSTIK | 326044 | PGANI | 357842 | RVVET | 380603 | ALERS |
| 271715 | DLMEI | 326047 | TLWLS | 357844 | SDSII | 380604 | RPVPS |
| 271751 | IFGAS | 326050 | FGSSF | 357850 | YVNRD | 380612 | VFIYP |
| 271764 | PGKKK | 326052 | TSGIM | 357857 | RIKKN | 380621 | TGRKP |
| 271776 | YQIRI | 326063 | ELKAN | 357858 | KSPCT | 380622 | SPCLF |
| 271816 | QLSRH | 326070 | AKTSI | 357861 | SDIWI | 380627 | TLSSG |
| 271835 | RSTKP | 326085 | NGAQP | 357863 | TQMLP | 380631 | VSTHS |
| 271843 | QIESI | 326095 | SLSEN | 357873 | IDAEY | 380632 | RTQPE |
| 271857 | GNLRI | 326101 | EESKA | 357875 | VSELL | 380635 | HRAND |
| 271889 | HADEM | 326109 | VASVM | 357876 | EEDKK | 380636 | TCVMN |
| 271915 | SSSSC | 326110 | ARNNT | 357880 | QQQQS | 380641 | VSYYS |
| 271971 | LTMNI | 326150 | VGQQP | 357882 | PLSTG | 380645 | ALGSL |
| 272065 | LEKAH | 326159 | TVQAS | 357883 | SEFTH | 380648 | AAFTR |
| 272091 | KGKKK | 326170 | HVFRS | 357886 | SNGER | 380651 | GTSSH |
| 272102 | LRNQK | 326194 | SCVIL | 357887 | KIVIK | 380660 | TRIHP |
| 272117 | DAPLA | 326200 | ISGGE | 357889 | INEGL | 380671 | PSVKN |
| 272133 | TLVSS | 326215 | SLLSS | 357890 | EATSD | 380674 | EQCRW |
| 272134 | RRLQP | 326219 | TPSRL | 357891 | IPNSE | 380675 | REPLH |
| 272139 | RGSEA | 326225 | ALSRE | 357892 | INEGL | 380677 | LFMSV |
| 272163 | FPYIY | 326227 | EVRII | 357900 | KLQNY | 380679 | SPGPS |
| 272164 | YTCKG | 326232 | GKTSI | 357901 | DLPLD | 380682 | GTPSA |
| 272167 | VLERQ | 326238 | CNWII | 357904 | EIFKL | 380684 | LKIQK |
| 272190 | FALAR | 326240 | LQQLY | 357905 | EEEPR | 380689 | IVSSA |
| 272193 | FSGSM | 326241 | PASHQ | 357906 | DEGKL | 380690 | FFRKI |
| 272217 | KSRRS | 326244 | GQGAL | 357907 | QAHRY | 380692 | ARFFV |
| 272223 | SKIKC | 326247 | DIEVI | 357909 | GRFNF | 380701 | RRQGE |
| 272224 | ACGCR | 326249 | HIGEK | 357914 | ATVSS | 380702 | EKRAE |
| 272233 | CCKVL | 326253 | SGSGP | 357915 | SGLGE | 380706 | WARPL |
| 272238 | LSLLD | 326259 | SKGTG | 357917 | ELDLL | 380707 | TVVLM |
| 272249 | TSSHL | 326267 | GQKEA | 357920 | PKSNL | 380711 | AETSN |
| 272252 | KFSVA | 326272 | VLHFQ | 357927 | GSGGT | 380714 | LVEGI |
| 272286 | PSQDW | 326281 | LTMYA | 357943 | RAEDM | 380718 | KIDEF |
| 272298 | MMTAK | 326294 | PCIIL | 357945 | HRKND | 380719 | SHYAA |
| 272317 | KPEDK | 326296 | DIHAQ | 357955 | GDPYA | 380727 | KDLQA |
| 272321 | HFGLV | 326301 | LSGKL | 357961 | FRADW | 380728 | SVDLI |
| 272322 | WEQKR | 326305 | ATPNL | 357962 | IKTHQ | 380733 | FVSNR |
| 272324 | CKFKD | 326309 | KHEYE | 357963 | IMQEY | 380734 | GYRRY |
| 272342 | PTSFL | 326333 | RPRPP | 357965 | RGRLL | 380753 | PCLCC |
| 272348 | ALERV | 326340 | VVLWQ | 357969 | TAVPL | 380757 | CQLDL |
| 272367 | WILSN | 326342 | LTLKV | 357973 | DRTKK | 380762 | RGGSQ |
| 272369 | QWHYM | 326348 | PSEDL | 357979 | KSSDI | 380763 | ITKGD |
| 272371 | KILGA | 326349 | GVNIL | 357980 | EEIDP | 380764 | IEEPK |
| 272386 | HTIVD | 326366 | HQFYI | 357981 | SYPEI | 380766 | LPSKG |
| 272390 | YTGHR | 326371 | DCTKY | 357986 | PVSDV | 380767 | RNIEV |
| 272395 | YTGHR | 326379 | STERI | 357990 | IFPAS | 380774 | FFPFV |
| 272402 | RPEGL | 326381 | VADLI | 357998 | EEDDD | 380781 | QSWFL |
| 272418 | KRAAT | 326391 | FREGT | 357999 | AFNKS | 380785 | PTDKH |
| 272425 | KVGSL | 326394 | AMVPS | 358012 | FVIFL | 380788 | GPLCQ |
| 272430 | LQSPV | 326404 | QGCTC | 358018 | KQAKF | 380790 | LGLQA |
| 272433 | YNKGL | 326407 | LGEGD | 358019 | QKPSA | 380793 | SKKAH |
| 272438 | RQKIY | 326411 | FTLDD | 358022 | AYLIR | 380803 | ETGML |
| 272440 | PCVLQ | 326417 | MKRHF | 358031 | RTGDL | 380804 | SSGCC |
| 272444 | WEWTQ | 326420 | AQEIG | 358033 | VTRFS | 380807 | TSPFP |
| 272452 | FHFQF | 326424 | LDGVD | 358042 | VSLKQ | 380825 | AASLD |
| 272462 | SIYYH | 326432 | VDYIL | 358045 | EPKEE | 380827 | LSVQV |
| 272520 | LLYPD | 326437 | IEHQG | 358059 | VINVV | 380828 | LKGFQ |
| 272521 | NPGRS | 326445 | DFSSS | 358060 | AEEIF | 380830 | KPRLY |
| 272542 | VILRM | 326473 | QTYNN | 358062 | MVLYT | 380831 | WHFGQ |
| 272546 | NVANG | 326475 | SEAAP | 358064 | FPPRY | 380835 | GTFNF |
| 272567 | VTNGS | 326477 | RKKIL | 358067 | CTSGH | 380845 | AGKLG |
| 272570 | KKSCK | 326491 | EDRNS | 358071 | EQPQH | 380852 | SLDGT |
| 272598 | YTGHR | 326495 | GYGWW | 358080 | FPNPK | 380855 | VNSAI |
| 272607 | SPHLA | 326500 | GAPNL | 358081 | NPAAP | 380857 | QKKTC |
| 272610 | INKVV | 326514 | IAQSH | 358089 | SYQTQ | 380858 | LKLQF |
| 272638 | STQQM | 326519 | RQRKY | 358095 | GMYFM | 380861 | KGKEP |
| 272641 | YLSSG | 326528 | DPING | 358099 | RIYNT | 380862 | DLLTS |
| 272643 | GDLDM | 326531 | FSLSF | 358104 | STGSS | 380865 | KVLER |
| 272644 | AKSEL | 326538 | WGPGS | 358105 | IPPED | 380868 | LSSRA |
| 272645 | RSFQY | 326549 | KDKLA | 358106 | VDWIP | 380871 | RNGQM |
| 272647 | YNHYS | 326550 | KMEKI | 358107 | ATTEA | 380873 | KQEKI |
| 272732 | VKVSS | 326563 | KLARS | 358118 | LTMVA | 380875 | KVSQL |
| 272748 | SLTAE | 326567 | KLAQI | 358119 | TAQFK | 380876 | GDGVA |
| 272771 | STRLI | 326570 | LTGSG | 358126 | SASRR | 380878 | KKAHS |
| 272797 | CVYNV | 326572 | SVLQK | 358131 | LVHRF | 380888 | IMNML |
| 272839 | QKEDL | 326574 | FLKIF | 358133 | KETMA | 380895 | TETEC |
| 272845 | MPTLR | 326579 | WRWTS | 358136 | WITRC | 380898 | EIEEK |
| 272847 | TEDVL | 326581 | NTHTG | 358140 | TTVRR | 380916 | FLYRI |
| 272852 | CMSLL | 326598 | LLAAL | 358142 | GQVLQ | 380920 | FRACQ |
| 272893 | EKKAM | 326603 | SKEDF | 358146 | TLGTP | 380921 | ALQSK |
| 272895 | DQMES | 326624 | ASFFP | 358147 | SRGYR | 380929 | KTDKN |
| 272902 | LPTMD | 326630 | AEHVK | 358153 | YGFGG | 380930 | FLTLQ |
| 272907 | LANRD | 326633 | RKMFG | 358154 | RGERA | 380931 | PKVLG |
| 272928 | EQSTK | 326649 | RIPLL | 358155 | KAKGK | 380933 | CYDIC |
| 272930 | KRYAR | 326652 | IGNSI | 358158 | KAKGK | 380935 | SRQRI |
| 272937 | VWRPW | 326671 | LATRH | 358160 | RGERA | 380937 | VWHLG |
| 272972 | REEFQ | 326687 | SFCSP | 358162 | YGFGG | 380939 | GVQWR |
| 273037 | LRKTS | 326693 | RHLQS | 358164 | YTSSK | 380941 | LCVPS |
| 273038 | PSVVS | 326699 | RVRKD | 358165 | PQGAT | 380942 | HSDKS |
| 273047 | QCCSN | 326706 | RRSAP | 358169 | NMGYM | 380946 | LRLIG |
| 273048 | AVAEP | 326708 | SLSGS | 358171 | GGTVA | 380960 | RIHPV |
| 273062 | PRPGS | 326718 | KLDVF | 358174 | EECIR | 380972 | VLFMF |
| 273063 | LHMPV | 326733 | EVILE | 358175 | RGERA | 380977 | PDFCP |
| 273064 | PLPPQ | 326756 | WRWQC | 358177 | PQGAT | 380981 | GMMAW |
| 273067 | RVTTV | 326759 | YDLDT | 358182 | QHLEE | 380982 | EKLST |
| 273075 | NLLVD | 326763 | ISTVV | 358200 | KFSNS | 380988 | TVPFF |
| 273077 | MHKSK | 326777 | QQRDK | 358202 | VTSDD | 380990 | IFLYF |
| 273083 | SPRML | 326785 | LTDKS | 358203 | VIFPQ | 380994 | VWKFP |
| 273085 | SDSDD | 326804 | YSYLA | 358205 | PILGM | 380996 | PQIIK |
| 273130 | EGEAS | 326806 | LAQNQ | 358207 | KKALD | 380999 | ACEKT |
| 273142 | LWKAH | 326809 | VTAGW | 358221 | VIFPQ | 381004 | FWYIG |
| 273145 | GNKSA | 326813 | NFGEV | 358223 | TLTPC | 381008 | MESTS |
| 273146 | RDEDP | 326817 | KKKFA | 358224 | DCGQF | 381009 | KKSSC |
| 273153 | EPVPV | 326819 | KLSNL | 358225 | PQPPE | 381010 | AGACA |
| 273158 | MGLKS | 326830 | LKKSI | 358227 | PSQRA | 381018 | LVSAS |
| 273173 | SSDDV | 326841 | EAVNS | 358229 | VIFPQ | 381019 | PPGFK |
| 273179 | RLCNR | 326846 | RATEK | 358232 | NLKPC | 381022 | ASSRK |
| 273183 | VLENI | 326858 | TLISK | 358233 | KQMAP | 381026 | FGAHY |
| 273221 | SVLCS | 326863 | RKFNS | 358241 | VGNEE | 381029 | NDSNG |
| 273258 | SKVKE | 326867 | CYLTF | 358251 | ASSTP | 381038 | KIPEE |
| 273261 | LAPKS | 326869 | GINKY | 358262 | RQLVP | 381047 | QEHAE |
| 273283 | VPDIF | 326870 | GDILD | 358265 | RIGTI | 381052 | MDKGF |
| 273286 | DSSPA | 326873 | EGVHR | 358272 | AIRSK | 381053 | CRRKR |
| 273291 | VPNLF | 326879 | ESPPS | 358280 | FLRRP | 381059 | SQSVR |
| 273308 | SHDEL | 326884 | QRDIT | 358283 | SVTEE | 381061 | PLTRI |
| 273317 | HQHHF | 326888 | DLNSA | 358289 | PQPPE | 381064 | LQTLT |
| 273320 | AWSVS | 326921 | KPGDL | 358293 | RTKKT | 381065 | PAPGD |
| 273347 | CPLET | 326924 | KKEKK | 358297 | DRWTF | 381069 | RKGDH |
| 273352 | AKESI | 326929 | VVSLL | 358301 | SESKV | 381076 | KVLGL |
| 273353 | KVQEE | 326933 | VLVGS | 358305 | IISLD | 381079 | PAPGC |
| 273359 | STIVN | 326946 | LAAAS | 358307 | LRYVF | 381086 | REIFF |
| 273371 | KIACV | 326948 | MSCSP | 358309 | TGIQV | 381091 | DERLQ |
| 273375 | SLHYD | 326966 | DCLLT | 358310 | EKLEQ | 381097 | PTAMA |
| 273390 | NENNS | 326967 | HLKTH | 358312 | LRLKP | 381102 | VQNFS |
| 273395 | PPLTI | 326981 | FDLEA | 358314 | HSSNC | 381105 | QRAKS |
| 273398 | RSLED | 326997 | PAQEL | 358316 | CWIIK | 381107 | SGIVQ |
| 273406 | LAKIA | 327024 | PRRRR | 358320 | DEAIY | 381108 | PFLSS |
| 273411 | TKLGL | 327038 | RSLSL | 358323 | NNNVQ | 381109 | ALFGK |
| 273430 | CFEVE | 327048 | SVFTK | 358334 | SLDVG | 381113 | RCESE |
| 273432 | HPSHF | 327054 | GCYVM | 358335 | RQGTS | 381115 | DICYS |
| 273435 | LELGI | 327070 | VKTLK | 358358 | VKFNS | 381116 | RTRQA |
| 273450 | VTFEY | 327072 | VNPRL | 358363 | QLPIC | 381122 | TPDLR |
| 273480 | QRIGK | 327075 | TISRP | 358365 | KALLL | 381127 | LGLQV |
| 273482 | FYPRN | 327080 | RSWRR | 358370 | DCGQF | 381129 | EPKQC |
| 273512 | LDQNL | 327082 | KSKTH | 358372 | TDPRD | 381134 | PWLGC |
| 273550 | SDNES | 327092 | FIILP | 358379 | RGRRS | 381136 | PDKAS |
| 273588 | YYTLK | 327104 | NLDPI | 358385 | GSGRR | 381142 | LVSNS |
| 273590 | KTHRE | 327133 | YYGSR | 358387 | DCGQF | 381143 | LVSNS |
| 273598 | LLSYT | 327143 | ADLHV | 358388 | IIQLH | 381145 | PRYHY |
| 273600 | CALDY | 327144 | KSNKK | 358389 | DNPPA | 381147 | YFVDA |
| 273610 | RVGHC | 327145 | KVKVP | 358391 | PQGAT | 381150 | EPALH |
| 273612 | LSLNE | 327154 | NEIHQ | 358394 | RGERA | 381161 | QPLVV |
| 273628 | ARTDL | 327168 | RTTAV | 358397 | EECIR | 381162 | KTSEF |
| 273641 | TWKPL | 327179 | KKFGT | 358400 | ASDHP | 381163 | LLELK |
| 273654 | VMSMS | 327191 | MSSLK | 358401 | GSGRR | 381167 | FLKNQ |
| 273666 | KWYQF | 327197 | CRLMD | 358405 | KSAWA | 381169 | LQRYR |
| 273668 | SDSDD | 327213 | KKESE | 358407 | KKQKK | 381181 | APDAL |
| 273695 | SLCEK | 327214 | AGPLR | 358413 | EKKNR | 381182 | RLFEC |
| 273739 | TRCVS | 327234 | DMLPH | 358414 | ARRPV | 381183 | ERPDA |
| 273783 | SSEDD | 327246 | EVSLV | 358417 | FQFHF | 381184 | KEHLS |
| 273784 | RHFKV | 327251 | EMSAL | 358422 | TRTEQ | 381186 | PSTAQ |
| 273794 | SPANV | 327255 | TDDMW | 358423 | PRVLL | 381188 | TLEHH |
| 273814 | ESDPR | 327290 | PKVLE | 358424 | KSKTQ | 381190 | ANGQA |
| 273853 | TQIKR | 327309 | RYGSY | 358428 | QFSRL | 381194 | GEAVA |
| 273854 | GMVPL | 327323 | LNTSQ | 358430 | ICAVL | 381198 | FQRKK |
| 273857 | TFLLN | 327336 | GNYKK | 358437 | SVHMV | 381203 | KRQSL |
| 273859 | KGKES | 327341 | SSAHA | 358451 | PSPDL | 381204 | HDAII |
| 273861 | AQTSL | 327344 | VQYQS | 358452 | APGED | 381209 | NENSQ |
| 273905 | ITSCE | 327348 | RPQNT | 358454 | SGQGV | 381210 | GVNLL |
| 273920 | LPSST | 327349 | FSSPR | 358456 | SGQGV | 381211 | EGSEV |
| 273936 | DEFMI | 327376 | KILLF | 358458 | WLPCN | 381215 | ERRRL |
| 273951 | LKNIL | 327378 | PSACL | 358460 | AKQKS | 381216 | QQQAQ |
| 273962 | NSLPH | 327379 | QRGLG | 358463 | WSIFS | 381218 | VALAN |
| 273963 | VVDCM | 327386 | PGEAA | 358464 | YRAMV | 381220 | SEPHS |
| 273968 | EVEQR | 327408 | EQLNK | 358466 | RRCLC | 381227 | KQQGQ |
| 273980 | PSPQI | 327415 | LYLML | 358478 | VLFGI | 381228 | ITFRK |
| 273986 | KKKEV | 327416 | RTYSC | 358479 | HKNSW | 381234 | ERDQK |
| 274000 | DQQTR | 327417 | QEHEV | 358485 | SMEMD | 381236 | IESLQ |
| 274008 | GLHTL | 327428 | GQRQL | 358486 | PSRHL | 381237 | EKKEK |
| 274024 | IFKKD | 327436 | AGVKE | 358490 | SPSSN | 381238 | DQGVH |
| 274026 | ETLNL | 327440 | ACGCH | 358491 | KSASK | 381239 | SLPSS |
| 274028 | GFPPR | 327442 | SDLSL | 358494 | TSITV | 381244 | CHPGH |
| 274030 | NNSLS | 327443 | GTHRG | 358497 | PRPLT | 381247 | GERKR |
| 274031 | ATQQK | 327453 | KARAQ | 358503 | VWCQR | 381254 | DKAGL |
| 274054 | FGPYY | 327459 | QNQGN | 358511 | SAEAL | 381255 | FCSLK |
| 274056 | EVVSV | 327467 | DDDDD | 358512 | QIGPP | 381263 | IRRNL |
| 274063 | RKLQC | 327468 | LRDGV | 358515 | TQMIK | 381271 | LELGL |
| 274068 | SLGLE | 327481 | FPAAV | 358518 | SGQGV | 381272 | VRKVL |
| 274071 | GSTGG | 327482 | TQELW | 358525 | AVRRA | 381274 | GWSAV |
| 274093 | HQQQD | 327486 | RAKIS | 358529 | SRDVI | 381277 | EDVEC |
| 274134 | EDVGH | 327487 | QDVGH | 358531 | EKLFV | 381282 | SSGGU |
| 274137 | RQHHH | 327489 | AGMIS | 358532 | PGTWK | 381292 | SVYMI |
| 274140 | QSSQE | 327506 | TSVGF | 358533 | LVFSE | 381293 | PLDFD |
| 274170 | SERTT | 327509 | ALTEI | 358534 | VPQHA | 381294 | SPGWP |
| 274181 | SKSNL | 327513 | VELPV | 358541 | SQAIK | 381295 | YGNGR |
| 274192 | IPFLF | 327522 | FIKIK | 358542 | VDPVH | 381297 | VYLDS |
| 274203 | QGSSR | 327525 | YSKIF | 358546 | AGVID | 381298 | RDQSK |
| 274217 | HIPVF | 327533 | KRQSF | 358547 | VSWVY | 381302 | TEGPV |
| 274258 | WALDL | 327536 | FTGEL | 358548 | PCVVM | 381304 | TRNKL |
| 274276 | GEHYC | 327538 | TREKP | 358549 | PVCRM | 381305 | GEKKT |
| 274278 | KVKKT | 327539 | GGWGM | 358551 | LKSTE | 381307 | CWVMW |
| 274289 | LQRCN | 327540 | HRFSS | 358552 | FLQKK | 381316 | SQGAV |
| 274306 | IKGAV | 327541 | ELIAL | 358554 | IRQDF | 381318 | FELQT |
| 274311 | SSEED | 327542 | AYFLP | 358558 | GNPRL | 381319 | LRRRR |
| 274341 | FRAYN | 327557 | RRMYL | 358563 | ELVSE | 381320 | LEVIP |
| 274345 | LICLL | 327563 | LGNNS | 358565 | RVIGV | 381321 | SAVIW |
| 274353 | KFKSQ | 327564 | FQRPK | 358571 | QYSYL | 381322 | RCPLP |
| 274361 | NTNVP | 327569 | LKEKA | 358575 | DVDET | 381323 | RKPLS |
| 274368 | CLSGL | 327570 | RQSVV | 358576 | YHKPC | 381324 | AEKAP |
| 274376 | TNDVR | 327582 | GQQLP | 358583 | ASLQG | 381325 | RPFVF |
| 274382 | LCGYH | 327583 | AEEKQ | 358588 | RQSKV | 381329 | HPSIQ |
| 274400 | APSGV | 327585 | FKRLT | 358593 | ENLES | 381331 | QLSNP |
| 274432 | MNEQI | 327588 | TTWVS | 358595 | YYHKS | 381336 | FTTLH |
| 274456 | DEENI | 327589 | GVTFT | 358596 | TATSC | 381337 | KDQRQ |
| 274457 | VSLHR | 327594 | MKKRH | 358599 | EKKDN | 381338 | LRPFF |
| 274458 | LDSLT | 327597 | TLQME | 358605 | HLMPQ | 381339 | DCGLF |
| 274459 | SQAWG | 327599 | KRHNC | 358607 | SLEQR | 381340 | EAAGY |
| 274473 | SSSSE | 327604 | CCIHF | 358618 | GKSLG | 381347 | LFVLE |
| 274487 | LQQKS | 327608 | RRCPG | 358622 | MECIE | 381348 | TQSFL |
| 274498 | YVEFL | 327611 | TSASR | 358635 | GQQWK | 381350 | NRDPG |
| 274507 | PTAYL | 327620 | TNGVR | 358640 | EESPV | 381351 | GDLSL |
| 274513 | FPMQL | 327627 | TFWEI | 358644 | QERLD | 381356 | GQAGL |
| 274516 | KKHQS | 327634 | LISES | 358650 | TTPAS | 381358 | KKSYS |
| 274520 | MRGKI | 327637 | MVKIT | 358655 | RSTAS | 381360 | PCVSS |
| 274532 | GLFTL | 327647 | LHMLE | 358667 | QRHRR | 381366 | VTVTG |
| 274542 | PVESA | 327650 | SEVKA | 358670 | CPASP | 381373 | LPPAL |
| 274545 | SSSVE | 327652 | GQHPT | 358677 | SAHMV | 381377 | HLQLR |
| 274547 | LYYVN | 327669 | RPKPQ | 358694 | ISKQH | 381381 | LQFVP |
| 274565 | HDGSC | 327675 | LKQIE | 358695 | HSFTA | 381382 | HMGQM |
| 274569 | VKTEL | 327680 | QRDAR | 358696 | VSSDS | 381388 | VFNVK |
| 274576 | DVHNQ | 327681 | EDRGA | 358698 | WLDQT | 381391 | LATKL |
| 274599 | TVVKS | 327687 | KETVA | 358702 | FRKTT | 381393 | SDPPA |
| 274605 | ESSKI | 327689 | QPAKL | 358703 | LTCEN | 381394 | LGRDG |
| 274606 | LPLPL | 327691 | TGDAK | 358714 | NCAYG | 381397 | KQRQF |
| 274615 | LCMCA | 327694 | LSAQF | 358715 | NCTSV | 381406 | PSFFF |
| 274625 | PTHPA | 327696 | SGFYY | 358716 | MHSNQ | 381407 | PLNIH |
| 274629 | LAAQK | 327703 | LLVCQ | 358718 | TKMTF | 381410 | PGLHL |
| 274643 | DFVTK | 327704 | DDMYL | 358719 | TKVDQ | 381412 | SVLPH |
| 274670 | SKMTS | 327715 | ARISM | 358727 | CDYGL | 381414 | TPDLR |
| 274673 | PIEQK | 327716 | LHPFS | 358730 | PTEQM | 381418 | ISACT |
| 274680 | VEGRF | 327718 | FVPSL | 358737 | AQPVM | 381419 | LTVSE |
| 274695 | VKVYN | 327724 | RCGTP | 358743 | VDEEA | 381421 | PGKGV |
| 274705 | KAPPF | 327732 | AQSPL | 358753 | SLYYN | 381422 | AGRWL |
| 274710 | TGPDT | 327738 | LNSHV | 358756 | GEAEQ | 381423 | QDSVS |
| 274711 | KECEV | 327756 | PALGA | 358758 | SCCSW | 381427 | FFVET |
| 274712 | TKKSY | 327758 | GIRAW | 358763 | SLGSL | 381428 | NSQDC |
| 274721 | LLSLW | 327761 | FINCF | 358765 | SNSDV | 381430 | VSWVR |
| 274764 | YTSSK | 327762 | LLLKY | 358770 | HSTGS | 381436 | ESPWY |
| 274766 | QVKEK | 327763 | KNTYI | 358771 | WLPCN | 381437 | CPGWS |
| 274773 | LRIWP | 327764 | LELQV | 358777 | LTQIV | 381438 | IETLR |
| 274787 | MKSRP | 327777 | TTDWS | 358778 | KMHQK | 381441 | NMKHF |
| 274793 | IEKYN | 327786 | DSRQQ | 358783 | SILRC | 381444 | RAHLS |
| 274811 | PREAE | 327796 | CFPFS | 358784 | IFTDV | 381447 | IRRMN |
| 274813 | KQQSV | 327801 | VKDEL | 358786 | EKSRK | 381454 | CAGQC |
| 274849 | LVRDS | 327813 | PGWEQ | 358789 | LEKKN | 381457 | TGMSH |
| 274853 | ESCRR | 327815 | DSREP | 358794 | KNSLT | 381458 | GCILK |
| 274867 | NRLNY | 327821 | QRAKP | 358795 | TYRSS | 381461 | VLGDV |
| 274897 | DSKEQ | 327824 | HYMAR | 358798 | QHTDV | 381468 | YGKCD |
| 274979 | SQLQQ | 327845 | GMKAM | 358799 | ALTLL | 381471 | EEYFI |
| 274990 | NFPCG | 327846 | RKHKF | 358802 | LFLPH | 381473 | MFSCL |
| 275015 | LLCTD | 327850 | TSTHS | 358807 | LNWTT | 381474 | PGPCG |
| 275016 | YKQRI | 327875 | QDSAL | 358810 | EELLQ | 381478 | PIPCS |
| 275034 | NLIGW | 327879 | EFCSH | 358812 | SVLED | 381479 | IPGCP |
| 275053 | ENDNT | 327889 | LRQGT | 358814 | NYYRY | 381484 | EDPLL |
| 275072 | VNAVE | 327890 | VVQKT | 358815 | KIYRP | 381486 | SGVDG |
| 275159 | RLQST | 327916 | QEWGK | 358820 | LGISP | 381487 | DLCTK |
| 275162 | LLSLD | 327917 | DQYCC | 358825 | RALHS | 381488 | QPSCC |
| 275169 | SPSEV | 327934 | PPRRP | 358827 | TWNSK | 381489 | CGSSC |
| 275189 | KSKPL | 327943 | WGFYA | 358831 | KAKGD | 381491 | HSQAS |
| 275198 | FSEHI | 327947 | KPHLY | 358838 | VWGNK | 381495 | RTSSC |
| 275200 | SLFLE | 327950 | LTPTP | 358841 | ASRSY | 381497 | LAKLK |
| 275209 | GCKGS | 327959 | AKLTA | 358845 | VWGNK | 381499 | SELGF |
| 275216 | LELSS | 327968 | CCPGP | 358851 | VWGNK | 381503 | RSSSK |
| 275227 | LPNET | 327975 | NVPEE | 358853 | SKAEP | 381504 | SHVPL |
| 275229 | LNSRI | 327988 | CLQEL | 358854 | AYSPQ | 381511 | KAATH |
| 275230 | LSPET | 327994 | QPTKL | 358857 | EGNPF | 381514 | ISACT |
| 275235 | LGPSS | 327996 | QELCP | 358858 | SPGPQ | 381518 | DIHAQ |
| 275254 | ESDSD | 328004 | NGESH | 358861 | EVALR | 381531 | LTVLS |
| 275358 | QIRRH | 328013 | GQTSH | 358862 | PLKIT | 381534 | VHVYM |
| 275364 | DIMLQ | 328016 | DEKVL | 358865 | SSQKI | 381535 | QNNNL |
| 275428 | ETQTL | 328017 | GLLCW | 358866 | RVVVP | 381536 | EPEGE |
| 275461 | KKESG | 328023 | ALMKA | 358867 | ECGLY | 381537 | SSDLR |
| 275493 | EFIGA | 328030 | FCLMP | 358878 | TPIVV | 381540 | GGGCC |
| 275513 | EKWIQ | 328036 | ELTIT | 358886 | KSKAH | 381542 | CQCKI |
| 275517 | DLLVE | 328043 | DALVR | 358888 | AVEKQ | 381545 | AHCFF |
| 275523 | EVDED | 328054 | SNVVR | 358894 | NEGAI | 381547 | FKNPQ |
| 275524 | SEENE | 328055 | LCRTP | 358896 | TKKSC | 381553 | SKLPP |
| 275525 | FNVQN | 328061 | TEKLN | 358900 | GVRET | 381554 | KAGVF |
| 275546 | LRLLP | 328062 | DPVVK | 358902 | FGKMS | 381558 | LTVSE |
| 275560 | LCQPS | 328069 | YGNNS | 358903 | AEGST | 381562 | STRPS |
| 275590 | YLRAS | 328070 | LLQLA | 358905 | SSSPS | 381566 | DWGTH |
| 275603 | SSLKG | 328077 | NLVKS | 358906 | LSQAG | 381567 | RQSLS |
| 275605 | GELEE | 328079 | PLNEF | 358909 | FNKGL | 381571 | PLLNI |
| 275635 | AATEA | 328082 | LPILH | 358918 | DSPLH | 381576 | AFLED |
| 275699 | ELKLT | 328083 | WSLPS | 358921 | HRKTF | 381577 | NQAAS |
| 275730 | TCTDL | 328085 | TKGSP | 358925 | PGPTR | 381578 | KQRLR |
| 275732 | SVDDY | 328088 | GQLTR | 358931 | CMLLN | 381579 | ITHRM |
| 275747 | WIFGE | 328090 | VYLGA | 358932 | LPWRL | 381580 | HFPLF |
| 275763 | TFLLR | 328100 | PRRRR | 358939 | EVTDA | 381583 | EMIIS |
| 275764 | DDEDL | 328103 | LEYRQ | 358945 | SVSPA | 381585 | IILLS |
| 275766 | VSCEK | 328110 | KAPRA | 358948 | PADGP | 381589 | PTSNQ |
| 275767 | KLESC | 328115 | SINKS | 358951 | SVSPA | 381590 | TPRCL |
| 275815 | QGFKD | 328118 | SAQES | 358953 | KYRFH | 381594 | KILKQ |
| 275820 | ASLRM | 328133 | RAWIS | 358956 | RKKHL | 381597 | PWDWE |
| 275874 | THCTC | 328137 | SQACY | 358957 | PADGP | 381599 | AQFPG |
| 275884 | FLHKK | 328144 | FRGKQ | 358963 | FKCTS | 381600 | LGLQA |
| 275954 | YEDYY | 328149 | PYCWS | 358964 | GFFST | 381605 | ADHGQ |
| 275988 | LVKKS | 328150 | RESEI | 358966 | DWEGN | 381606 | HVYSH |
| 276009 | GFGLA | 328160 | PVALV | 358967 | SVSPA | 381611 | PRQLQ |
| 276014 | QGLVL | 328164 | GSNPG | 358970 | VNLQV | 381615 | TGDAK |
| 276033 | PEEPI | 328166 | PYKCE | 358973 | TERVS | 381618 | KRTHT |
| 276049 | RENSW | 328169 | YAGKE | 358974 | ANYGS | 381620 | KRKPP |
| 276052 | VDTEF | 328173 | TPRED | 358982 | WTYNG | 381621 | HKERK |
| 276055 | ADGAT | 328174 | SVWGH | 358994 | QSLLK | 381622 | LTVSE |
| 276062 | LPEDE | 328178 | KLRSS | 358997 | DEFQS | 381624 | LTVSE |
| 276072 | LDSDE | 328181 | CKCSC | 358999 | GGKTR | 381627 | SRGKV |
| 276077 | TPELC | 328182 | RKKRP | 359000 | TSEVN | 381628 | NPPSE |
| 276079 | KRRRY | 328187 | ENIDE | 359001 | KADGQ | 381630 | SSLEV |
| 276096 | KSKKN | 328190 | QMQDQ | 359004 | KSADH | 381632 | FFVET |
| 276101 | ELEII | 328202 | VRRVS | 359006 | EVDCS | 381636 | NHPGQ |
| 276110 | LKPET | 328203 | TFLWS | 359018 | NRLKY | 381646 | PKERS |
| 276127 | SSSGN | 328207 | DGVGS | 359019 | VERPV | 381651 | HCARP |
| 276173 | DEEEK | 328215 | LLKRL | 359022 | KTKSQ | 381652 | RASIN |
| 276185 | KTLIK | 328216 | GSHYA | 359024 | KFDSE | 381653 | SKKKK |
| 276198 | RISSV | 328220 | QHPTT | 359025 | LIKDE | 381654 | ASFNA |
| 276201 | EGGEE | 328228 | GERKQ | 359031 | NSISD | 381655 | AAFWG |
| 276204 | RYAEV | 328230 | ACAES | 359032 | SDSAS | 381658 | SLGLG |
| 276211 | SYFFP | 328232 | RFSRR | 359034 | KRNEE | 381660 | TEARV |
| 276218 | SEFDG | 328236 | LMGLK | 359042 | KDETV | 381661 | RGPGK |
| 276241 | RSQSI | 328243 | GKNPT | 359045 | TPKPH | 381665 | VQHLE |
| 276282 | KHRNQ | 328245 | LRIHT | 359046 | EQFEF | 381666 | SPKEK |
| 276297 | DTKSR | 328251 | LIQVV | 359050 | FSTIS | 381669 | SPGCL |
| 276326 | DLFKF | 328264 | TAPLP | 359052 | VVIKR | 381670 | SLGLG |
| 276344 | EEEGV | 328270 | ASSCC | 359053 | QRMIG | 381674 | ISLAR |
| 276345 | DTEIP | 328271 | CCSHF | 359055 | QRMAG | 381675 | DLLEI |
| 276369 | VYHMY | 328277 | RTGAS | 359067 | ADITI | 381679 | NTERS |
| 276373 | PDHEE | 328307 | ASKED | 359070 | PGPLL | 381683 | GKAST |
| 276390 | DSAKH | 328325 | RFENN | 359073 | VEEDE | 381684 | CFTNK |
| 276410 | NTGKS | 328326 | YETTL | 359085 | SPLVF | 381688 | LKCSG |
| 276414 | GQKKI | 328327 | KFDDI | 359095 | FAMED | 381689 | DLQHW |
| 276416 | IVADD | 328335 | LQVYI | 359096 | AESKL | 381694 | PYKCK |
| 276420 | KKGPK | 328336 | LVRKK | 359098 | PKKQE | 381695 | EDENQ |
| 276431 | DSAMS | 328340 | KQVKV | 359100 | AESKL | 381697 | FLKPA |
| 276440 | EPGSQ | 328347 | SKPSR | 359107 | SFSRL | 381703 | KLHQK |
| 276449 | SEARC | 328352 | RFVRH | 359115 | LSHSV | 381706 | ANCYM |
| 276461 | ATKEN | 328358 | SSQRY | 359119 | PWLNP | 381707 | AAECG |
| 276480 | KGIHV | 328359 | WAFCC | 359123 | SINGL | 381710 | VGESW |
| 276497 | YQQQP | 328364 | ADFFL | 359128 | QGGRP | 381711 | RPSPP |
| 276530 | MSSGR | 328370 | SEQFK | 359131 | YKRKI | 381712 | SEPWV |
| 276533 | AVQLI | 328376 | AATPS | 359149 | DFQNP | 381714 | LTVVE |
| 276569 | RSFYW | 328397 | FCCRS | 359151 | LLDLK | 381715 | SHHTW |
| 276570 | YCTDS | 328400 | WPILL | 359155 | MALEI | 381716 | LIKHK |
| 276571 | EIIGK | 328402 | KKKKH | 359160 | PETTA | 381719 | NQTME |
| 276585 | EHHEK | 328403 | IKVPS | 359170 | DCKTQ | 381720 | KECTL |
| 276590 | WKAHL | 328405 | FGFLS | 359171 | QDTNV | 381724 | FGLSD |
| 276594 | FHEDY | 328409 | SNLCA | 359172 | NPTKA | 381725 | LGPGS |
| 276603 | SDSED | 328410 | TSEEV | 359196 | AGPPS | 381730 | ILTGL |
| 276616 | LSALL | 328411 | ATSNS | 359204 | RAYKD | 381731 | GEIVE |
| 276646 | FRIKT | 328412 | QYGII | 359206 | TYISR | 381734 | IFCHC |
| 276651 | KQAHP | 328422 | IRWNR | 359211 | VNPVC | 381738 | ERGSP |
| 276654 | ALLLC | 328423 | AGLRN | 359212 | IDVDD | 381741 | LFMDE |
| 276658 | GVKLS | 328426 | RSSYW | 359215 | ASACE | 381744 | ILTGL |
| 276659 | DRANQ | 328429 | RPLHK | 359216 | SKSTI | 381747 | ALPKQ |
| 276681 | RRWRP | 328430 | GYKLT | 359219 | NDDYY | 381752 | DKAFN |
| 276682 | QEYNN | 328432 | RPSCC | 359221 | LALVK | 381754 | AQRDA |
| 276689 | RERPM | 328444 | ASSCC | 359222 | NGKLD | 381758 | MTMWG |
| 276692 | FFPGI | 328452 | RNPSR | 359224 | LGRIV | 381759 | FIEGH |
| 276704 | IQILA | 328455 | LLSKC | 359225 | QGIDY | 381761 | LDIEI |
| 276708 | QLAEA | 328464 | QLREE | 359231 | ELMEK | 381766 | ESTKM |
| 276737 | LNYFK | 328472 | EPAAD | 359233 | VEFGS | 381767 | HHTWP |
| 276770 | PRSQD | 328478 | AADEP | 359234 | PCCPG | 381770 | PQPPE |
| 276779 | PRSQD | 328484 | HHKTK | 359240 | TATEI | 381776 | VEMPL |
| 276816 | IHTRE | 328487 | SAASL | 359257 | PASGL | 381778 | WPSRN |
| 276844 | AGQSP | 328495 | AGDSQ | 359258 | KRRGS | 381780 | LIQKR |
| 276893 | YSKGR | 328511 | VETDV | 359262 | SVKAA | 381784 | LTAIS |
| 276914 | EHKTH | 328515 | VHGAE | 359275 | AVTYI | 381785 | MNARL |
| 276925 | TATGD | 328521 | GKVQP | 359277 | APKLG | 381786 | DRFLQ |
| 276927 | LRRKE | 328524 | KGIDI | 359279 | APMRF | 381788 | QNSRP |
| 276943 | LFRRK | 328545 | QFKTK | 359282 | EECDN | 381793 | IRVAL |
| 276960 | FCTIL | 328547 | YFACE | 359284 | EECDN | 381796 | LGHPI |
| 276978 | LWEGI | 328551 | MCAVQ | 359285 | LAGMI | 381798 | GCFNI |
| 277010 | FGQDP | 328552 | FPEVT | 359286 | KSKAK | 381800 | PELMS |
| 277082 | KTGSH | 328553 | CVKYS | 359290 | DDDDI | 381802 | GGALG |
| 277120 | LDILG | 328563 | RILIQ | 359291 | YEKLF | 381804 | QFPDL |
| 277165 | LNKEE | 328570 | DQDSK | 359292 | SYCLK | 381805 | MSSYV |
| 277216 | KAINQ | 328580 | GVSHT | 359297 | LRRVK | 381806 | GASQP |
| 277225 | EAKKE | 328592 | RRVLW | 359300 | KTSLY | 381811 | PPGSG |
| 277239 | RLDPE | 328598 | SQEPS | 359301 | REGEE | 381813 | LGPGI |
| 277244 | KQEAK | 328605 | HSSHT | 359310 | TKGSP | 381815 | GFADY |
| 277309 | SASDS | 328610 | VRIEK | 359313 | RKSRT | 381817 | PGLES |
| 277458 | VEDDI | 328614 | SSPGA | 359319 | ATPPY | 381823 | QQVNK |
| 277491 | ITSPE | 328625 | WSSVY | 359321 | GATAS | 381826 | MGTCL |
| 277508 | EPCRF | 328631 | LVNKF | 359323 | QEREL | 381829 | IFQDC |
| 277517 | KIHRV | 328640 | LYKTN | 359333 | YTNDS | 381830 | APFCL |
| 277526 | PPMRE | 328671 | ATLSC | 359334 | MIRKQ | 381832 | VDLFH |
| 277531 | SDQDG | 328672 | SSVLD | 359339 | VFDEF | 381835 | FFTKT |
| 277540 | EWEGN | 328673 | AEGQY | 359340 | VYLPA | 381845 | QYWRK |
| 277549 | QDHWC | 328674 | TKNYV | 359345 | RAAES | 381848 | KINPI |
| 277554 | YAGTL | 328677 | LLPFL | 359349 | NGLKT | 381851 | SDSHF |
| 277570 | LRESS | 328690 | STSAN | 359353 | VCLNL | 381852 | HHAQP |
| 277575 | ESVLL | 328694 | HVKER | 359356 | YSTTV | 381853 | PPGFK |
| 277598 | KPRKH | 328695 | QVETR | 359358 | SRDRQ | 381856 | PQPGV |
| 277632 | SPSLN | 328708 | SSSAY | 359361 | PKKFH | 381857 | AVLIG |
| 277668 | HFDTI | 328720 | PGTGE | 359366 | SSVAY | 381860 | LVLST |
| 277705 | VAKTI | 328722 | NVTRP | 359368 | SCLPE | 381864 | RLGAF |
| 277746 | GFMNN | 328729 | KLERI | 359369 | ALLLC | 381873 | LGTYY |
| 277865 | GVTFT | 328732 | SEANN | 359370 | HKLLV | 381875 | ADTVL |
| 277882 | AKCEG | 328736 | AFRGS | 359371 | CHEKP | 381878 | ITLKL |
| 277895 | KAKLF | 328737 | FAFRD | 359372 | FGSRV | 381883 | LTLRS |
| 277903 | SESGF | 328738 | KIGRT | 359375 | LWMNQ | 381889 | GQVPS |
| 277942 | PAWDI | 328747 | SQSQS | 359376 | THEDL | 381892 | TIEDD |
| 278025 | VATGC | 328768 | RLFQA | 359378 | PGADK | 381893 | SEAEV |
| 278060 | PRPRL | 328773 | SKFGD | 359380 | NYKSG | 381894 | IQGSH |
| 278070 | KNLRR | 328777 | MLLTL | 359382 | SNKPC | 381895 | KHQMK |
| 278174 | QNPML | 328783 | DQYCC | 359385 | NIGIK | 381897 | CLVVR |
| 278175 | APHFL | 328788 | CLAFL | 359386 | EGKQD | 381898 | CEFIA |
| 278187 | KKEIK | 328800 | SQVHS | 359388 | QDPVA | 381900 | EVFQE |
| 278193 | RRQQT | 328801 | FLNDI | 359392 | PVTRL | 381903 | CEFMA |
| 278198 | QETQI | 328813 | KAINV | 359393 | EEEDE | 381906 | NSLRP |
| 278200 | RFSDD | 328818 | IEESC | 359396 | GPWMK | 381914 | KVLGL |
| 278207 | LPEKY | 328829 | LCIWH | 359401 | SVNPN | 381921 | NTCSG |
| 278222 | LPKKY | 328835 | EDFEF | 359403 | EPARI | 381922 | SMQYI |
| 278243 | EEKRF | 328847 | FSSDV | 359404 | LETQH | 381925 | CQALF |
| 278279 | IRHLK | 328854 | HFTLS | 359410 | TRKKD | 381926 | STLGG |
| 278282 | SSLCN | 328858 | PAGKD | 359411 | SSLYP | 381930 | IWSYS |
| 278314 | RRGNL | 328866 | SLDDC | 359417 | VHTSV | 381932 | PVASS |
| 278319 | TRDKS | 328874 | FQCTD | 359424 | SWLTE | 381935 | CVEVS |
| 278353 | KTKKN | 328875 | VLKED | 359428 | YGNDP | 381937 | QGLFR |
| 278359 | ENGIV | 328878 | LKTNH | 359433 | IRDML | 381942 | TTGAR |
| 278360 | ERNMK | 328879 | CVLDL | 359436 | KESTG | 381948 | TQGQT |
| 278379 | WKREK | 328889 | QERLR | 359440 | PEESF | 381951 | RTSEA |
| 278385 | MKIGV | 328901 | APSGV | 359444 | ADQST | 381953 | ISALF |
| 278407 | YDPRA | 328911 | RRKRN | 359446 | QRGPA | 381954 | ELKPL |
| 278409 | TSSFL | 328915 | SHCLC | 359449 | IEMPD | 381957 | ISFLL |
| 278412 | SGSDE | 328928 | GPRAL | 359454 | FDGIF | 381959 | LVRRL |
| 278426 | SEVTA | 328934 | ATSDA | 359458 | FYFSE | 381960 | KQKCQ |
| 278456 | NGYIA | 328938 | KTIFQ | 359459 | YIYIY | 381961 | LHQWH |
| 278483 | LFWKT | 328939 | LSSSS | 359460 | PEESF | 381962 | LGLQA |
| 278499 | TRHLT | 328940 | DLLET | 359462 | GVQPP | 381968 | FELED |
| 278505 | NSGEL | 328952 | DLNEM | 359464 | VVLVQ | 381969 | FSNPV |
| 278520 | EKFEL | 328957 | GKSPK | 359474 | TPHPR | 381970 | LEYLS |
| 278544 | PLRKH | 328968 | RESIV | 359478 | NSTKF | 381975 | ERLQP |
| 278550 | EMGRR | 328980 | RIHIR | 359483 | QMKTE | 381977 | PCLCL |
| 278568 | TDCCS | 328983 | SHPRN | 359484 | TQLWK | 381980 | LPATT |
| 278572 | PVPTA | 328986 | TTTTV | 359485 | HKLPF | 381982 | IVVGS |
| 278590 | SQLGY | 328989 | FILWP | 359489 | DAHLL | 381986 | SSSFS |
| 278601 | NGTLV | 328990 | AHPSH | 359490 | LGSRI | 381987 | WLRDT |
| 278612 | LHYDE | 328992 | LIEGK | 359497 | ICNIL | 381989 | KATNE |
| 278616 | WKAWV | 328998 | QTSLR | 359498 | EHGDE | 381993 | KGKIF |
| 278618 | NGTKS | 329000 | VDFRP | 359504 | ACTIS | 381998 | LSSLE |
| 278671 | QFGIP | 329008 | SGGSQ | 359505 | RCIDF | 382000 | PRTGS |
| 278715 | LNDAH | 329009 | SQDGR | 359506 | VNGVP | 382001 | NRLAA |
| 278723 | SSEPE | 329029 | EDDGY | 359507 | KNILV | 382003 | WAVRR |
| 278742 | ENTGV | 329033 | LKCFD | 359512 | HKLKI | 382006 | NMAKP |
| 278752 | KNDEL | 329034 | DKKAQ | 359518 | KRRRT | 382011 | RKKVP |
| 278765 | EPAGY | 329036 | PLPPP | 359520 | VQVGP | 382017 | SQASL |
| 278772 | HQRTH | 329040 | DIKYR | 359521 | AISQL | 382021 | KEITY |
| 278779 | KPHHF | 329051 | VAAPA | 359524 | ENDQS | 382027 | IAISV |
| 278780 | EEERK | 329054 | GLEDK | 359525 | AMNSI | 382030 | LKENF |
| 278816 | LRLRR | 329056 | ISGDK | 359526 | WRAGK | 382031 | LENIA |
| 278823 | IVLED | 329069 | AGLPT | 359529 | EGGED | 382033 | WRALG |
| 278826 | MQVRR | 329080 | QFLPL | 359531 | KLPQL | 382036 | WRALG |
| 278833 | DLSEA | 329106 | PRSQK | 359532 | RETLY | 382040 | HSRDG |
| 278836 | YRLCD | 329112 | DETHS | 359534 | DSGED | 382042 | WRALG |
| 278840 | AYLHK | 329115 | HHNSM | 359535 | NAVSN | 382045 | GSDSE |
| 278845 | SSLDV | 329117 | GNLAK | 359539 | VTKIQ | 382046 | HSRDG |
| 278853 | ESNRQ | 329123 | KSRGR | 359540 | NVTVQ | 382050 | EKITS |
| 278855 | SPNLL | 329124 | SYLVS | 359546 | EIPAK | 382055 | LLQLA |
| 278856 | GSTSP | 329127 | PELQP | 359551 | HLRAQ | 382074 | RSVAR |
| 278865 | PPNSV | 329137 | LLLGT | 359552 | IADWC | 382075 | WAVRR |
| 278882 | DREPN | 329140 | VNTTF | 359553 | RAQWL | 382076 | TPGQE |
| 278886 | AALSV | 329141 | GNYSG | 359557 | EEDDY | 382080 | SSKDL |
| 278888 | PPIDL | 329144 | EAKKS | 359561 | ERPKK | 382086 | SSSPH |
| 278903 | ATAGH | 329153 | SASDK | 359563 | IDWKI | 382093 | RVFRY |
| 278911 | REEPS | 329161 | EAPEM | 359566 | HVCTA | 382097 | VPVDQ |
| 278916 | WLPAT | 329163 | QWPVA | 359567 | PLTHI | 382098 | LLSLL |
| 278919 | VLCPT | 329165 | QSRPL | 359571 | VTLND | 382099 | TFVLQ |
| 278921 | AIFAF | 329170 | QTPVQ | 359573 | DQKVC | 382105 | PQLPE |
| 278927 | AGSLV | 329189 | QTAFT | 359581 | YPKSN | 382107 | SREGV |
| 278934 | MQTHV | 329190 | RARLS | 359583 | ACRYR | 382108 | KTKKK |
| 278935 | KVYRF | 329200 | FLEEI | 359596 | GTKLL | 382109 | ASFQA |
| 278937 | LEDTD | 329205 | VESVM | 359598 | VTMFE | 382110 | AYYET |
| 278947 | DDGAK | 329210 | QKPPY | 359601 | SVYSA | 382111 | LIHTA |
| 278949 | YEETY | 329212 | QSAGR | 359606 | VGNEN | 382113 | TLRAP |
| 278951 | AGPVT | 329213 | AMFET | 359607 | DLKSM | 382114 | RGIQV |
| 278968 | RQIIS | 329214 | NWTLS | 359610 | MALLY | 382122 | KAFST |
| 278976 | SLYLT | 329229 | EVHPS | 359622 | HDTDT | 382123 | LHGSS |
| 278979 | SSCTR | 329233 | PLPPE | 359629 | CEPSC | 382124 | ILQQN |
| 278980 | MECFC | 329243 | RSARD | 359634 | GAGAR | 382127 | LMTSD |
| 278990 | DAPQM | 329246 | TMVKQ | 359635 | HNEST | 382128 | KETPF |
| 278994 | DLLKE | 329250 | GQKRK | 359640 | LIPIS | 382131 | GKYVD |
| 279022 | KDKDD | 329256 | FPPGF | 359643 | NKSTS | 382133 | DFQRE |
| 279024 | PLASL | 329264 | HLLEH | 359645 | GRSRY | 382134 | VSFLL |
| 279027 | TFRTL | 329265 | EPAGY | 359649 | VPGFK | 382139 | ARQHG |
| 279028 | HDRPG | 329266 | GARRP | 359650 | VPPFK | 382141 | KKIAS |
| 279036 | GVPPL | 329271 | GRCWH | 359651 | GYCYD | 382145 | KGNQI |
| 279052 | LGKTT | 329275 | QPALA | 359652 | DLHLV | 382146 | SLTIH |
| 279058 | YKPPR | 329287 | PEVHG | 359655 | GHTFD | 382147 | QVRSR |
| 279067 | ELSPC | 329292 | ADIHV | 359663 | GLLKL | 382149 | TTEWS |
| 279101 | LTQQF | 329295 | YYPSG | 359664 | KLLQR | 382154 | WDDTA |
| 279146 | GIFSH | 329301 | KSHKE | 359665 | FFSWW | 382155 | REMEA |
| 279147 | SGLSF | 329303 | GEAAT | 359666 | AFVSP | 382157 | DLQSV |
| 279168 | RLSRY | 329312 | PAECS | 359668 | YLQHL | 382160 | RQTTR |
| 279178 | EVTQF | 329318 | VASQN | 359674 | PNRRH | 382165 | WRPKI |
| 279206 | LLPPL | 329343 | LLPDD | 359675 | VCSFL | 382167 | KERKK |
| 279227 | GHEAF | 329353 | LPLGS | 359680 | SNGWL | 382169 | PMGTV |
| 279230 | ENTQL | 329355 | DQAVS | 359682 | AEDRF | 382172 | IEKLL |
| 279242 | LEKGF | 329356 | PCVSS | 359685 | NCCIQ | 382178 | LSPRR |
| 279247 | LTMFA | 329357 | SGNGF | 359686 | YCSSP | 382186 | LSSWD |
| 279249 | MQIPT | 329360 | PEPQL | 359688 | RKFHL | 382189 | ARISL |
| 279259 | PNANS | 329361 | LIFYA | 359691 | LEYFI | 382190 | PSSWD |
| 279263 | MPYIY | 329365 | KTIHT | 359693 | LLKLE | 382191 | RGSHL |
| 279281 | ICERG | 329372 | FPRAH | 359694 | DTFRQ | 382193 | VRVPQ |
| 279386 | SKPMY | 329374 | NPTEA | 359698 | PPPQD | 382196 | EIPAK |
| 279387 | ADYFL | 329380 | SGHSL | 359699 | YESCK | 382197 | QVIQF |
| 279392 | DGESN | 329384 | RNACI | 359709 | AGSCL | 382200 | FFYFL |
| 279396 | SRTRL | 329395 | GLEKD | 359719 | EFGEF | 382203 | LLRSL |
| 279434 | EAAHH | 329403 | AGSQS | 359727 | ESRFN | 382204 | DMEEN |
| 279441 | SWLHC | 329411 | LEQPA | 359729 | RHGPA | 382208 | LLSTF |
| 279463 | PSTSW | 329418 | FPFQI | 359740 | SLEEM | 382211 | NDTAS |
| 279477 | WKQKA | 329419 | DILDD | 359741 | RRQGC | 382212 | PILLC |
| 279488 | SLQST | 329426 | RETSM | 359752 | LVTSL | 382213 | STSDD |
| 279544 | WICQY | 329428 | YNDKF | 359754 | LKRMI | 382215 | RKYIL |
| 279573 | GQPPQ | 329431 | LYEVM | 359759 | LKRMI | 382218 | KKQEA |
| 279593 | IESDV | 329435 | RAHLS | 359766 | LKRMI | 382219 | WHKSW |
| 279716 | SATTC | 329448 | RRRSQ | 359768 | IWNLK | 382222 | ERLHT |
| 279783 | FKIPI | 329452 | LQENW | 359769 | LKRMI | 382230 | KDSFK |
| 279791 | TYVRQ | 329463 | FCDHT | 359772 | LKRMI | 382235 | PTNFK |
| 279804 | PGGSA | 329466 | RKKVT | 359777 | LKRMI | 382237 | SHHTP |
| 279839 | EKYIK | 329467 | LCKEK | 359778 | FGCLR | 382239 | KLTLL |
| 279873 | PSTKL | 329471 | LSLPA | 359782 | SQDIK | 382241 | QQKCS |
| 279907 | KMTVE | 329482 | YKRPF | 359783 | AQGKK | 382245 | ASQAA |
| 279934 | RRSSL | 329492 | GGLLK | 359787 | LQPCI | 382246 | SLSSE |
| 279968 | PLHSY | 329499 | PSFSP | 359788 | SRSSC | 382249 | MTVSS |
| 279992 | KRYRE | 329503 | KKWDH | 359793 | KSAST | 382250 | KKRKK |
| 280056 | YKQGN | 329507 | KGENQ | 359795 | KRRGL | 382253 | DSDSG |
| 280057 | EEFYI | 329522 | RTLIS | 359799 | HLPAS | 382267 | ATNNS |
| 280082 | ATKYV | 329528 | LLDMI | 359802 | LVSPN | 382273 | RKLVQ |
| 280083 | PQQET | 329539 | VIEVS | 359803 | HHHLY | 382277 | AFGGQ |
| 280097 | IVIEA | 329548 | TVQKK | 359805 | KSSKR | 382280 | SRLLN |
| 280098 | RLKQS | 329551 | LFSEK | 359810 | LWTLK | 382285 | TFTKL |
| 280115 | VLPLL | 329553 | QREEE | 359813 | AILRT | 382298 | ARLEF |
| 280154 | KPESY | 329554 | RGERA | 359820 | RQTFN | 382302 | TPDLK |
| 280155 | RKRIV | 329557 | STQSG | 359827 | EAKVQ | 382303 | FWDKV |
| 280187 | LNAYT | 329558 | SQRRL | 359831 | LQGQR | 382307 | LANFF |
| 280190 | RLNPF | 329565 | RAHLS | 359837 | FPANR | 382308 | MVSIS |
| 280191 | MDKKP | 329568 | CDFFC | 359845 | TGDFF | 382310 | GKLGQ |
| 280193 | PQMMS | 329584 | IDIRD | 359848 | MKFIF | 382320 | KERKK |
| 280200 | PKTRV | 329586 | TYGLQ | 359864 | QREWR | 382328 | KKKIS |
| 280236 | ECKEP | 329603 | FRSPR | 359866 | SCGCH | 382329 | ACQWS |
| 280245 | MKSFL | 329612 | TGGKE | 359869 | TLQKE | 382332 | LVAQR |
| 280258 | DCREG | 329614 | LFGGS | 359871 | DKYKN | 382334 | KVLFT |
| 280295 | EQRDH | 329617 | AHVPE | 359885 | GNYQQ | 382335 | QSRVR |
| 280326 | GESEE | 329623 | YLGHK | 359890 | NQLKF | 382339 | LASAA |
| 280330 | LPIQA | 329626 | GCWQK | 359891 | SVMAV | 382342 | DAGLV |
| 280346 | ITMLL | 329632 | NPAFF | 359892 | QKQKE | 382345 | RSLHL |
| 280350 | TMPLV | 329634 | ALEHF | 359899 | PLQNW | 382346 | LHCYH |
| 280354 | QKGGQ | 329638 | STHLN | 359904 | FPEDY | 382349 | EWRGR |
| 280357 | VQNED | 329640 | CGGDD | 359910 | QKKAS | 382354 | VITLL |
| 280358 | NTLHR | 329647 | YDVFN | 359924 | ADESP | 382355 | TPRQS |
| 280362 | VYKGE | 329654 | KNSIK | 359925 | VTTSV | 382356 | TCIQG |
| 280377 | CMHTN | 329662 | PKGRK | 359928 | FEDSS | 382360 | AAFWA |
| 280481 | DSSEV | 329663 | GPHSS | 359939 | EFLQT | 382362 | IKGKK |
| 280527 | FYQTV | 329668 | LHSNK | 359940 | LPVPI | 382363 | GVSQC |
| 280557 | GEVKK | 329674 | SGSPD | 359942 | VPVSI | 382364 | KEKKK |
| 280560 | GSHKA | 329684 | TVDVA | 359956 | LKSKN | 382368 | MQQKR |
| 280571 | SGKQT | 329685 | KLGLF | 359972 | SVQLS | 382374 | HGPQH |
| 280576 | TLNSD | 329686 | FQNVM | 359976 | SGSHS | 382375 | SNDLY |
| 280605 | LTLMK | 329689 | FAQGE | 359980 | QMLKI | 382382 | DLTQD |
| 280606 | QPTSC | 329697 | PHSSS | 359982 | RFPDL | 382383 | RPSPI |
| 280612 | EEDKL | 329698 | LKLVE | 359988 | MDMSD | 382385 | NCIHI |
| 280614 | SDGLS | 329715 | QIVKK | 359990 | DRIPT | 382387 | GRMGM |
| 280663 | PQLLR | 329735 | DVSYL | 359991 | GKAKK | 382388 | RVIAD |
| 280665 | MKKTM | 329736 | REGEE | 359994 | RREGL | 382389 | CLSLV |
| 280684 | MLTEV | 329738 | THTRV | 359998 | NIFRP | 382394 | DMFSV |
| 280696 | EVFED | 329748 | LQTQI | 360003 | DEECG | 382395 | LVIAT |
| 280700 | KFSDL | 329757 | ILSTK | 360009 | RKYQN | 382396 | SPSIS |
| 280701 | CIAGL | 329761 | EGQGS | 360017 | GCSLK | 382398 | HDEAC |
| 280704 | KDLIF | 329774 | NAIFG | 360018 | LCSFP | 382399 | LGLQA |
| 280706 | KELKL | 329777 | LPETL | 360020 | KCIIM | 382406 | PTDRH |
| 280734 | LTKAN | 329786 | RPAHR | 360021 | TCIIL | 382407 | NTCGK |
| 280749 | SKKKK | 329793 | EKLQI | 360025 | NLPER | 382415 | TPGLK |
| 280756 | KDKSD | 329794 | RPYPI | 360031 | KDRRP | 382416 | SPLAW |
| 280758 | KGSVV | 329795 | GIRGR | 360034 | FPALA | 382418 | LPTAA |
| 280772 | KKSHS | 329797 | KEYFI | 360035 | WIHFI | 382422 | QRGSA |
| 280800 | KVSWD | 329803 | FKYII | 360045 | ILNAS | 382423 | FRTTW |
| 280871 | ASDVE | 329825 | LWEGI | 360054 | RFHRP | 382424 | GPWPQ |
| 280886 | VAYNM | 329846 | KHARH | 360058 | SGSLL | 382429 | CGKAF |
| 280892 | NRFVV | 329847 | SFGLV | 360060 | LVPGS | 382434 | GQCPS |
| 280904 | ACMKR | 329858 | CPSVG | 360065 | SNQSA | 382437 | QKTNK |
| 280961 | PDECV | 329860 | LQESK | 360069 | LVIEI | 382438 | QELNH |
| 280979 | RNMHR | 329865 | RVVCL | 360073 | KIFRF | 382441 | KGRKP |
| 280987 | VSVPP | 329869 | LPRAL | 360090 | KCSVD | 382443 | DSDSG |
| 281017 | RRKED | 329880 | SFRFP | 360104 | SWEQW | 382450 | LPVLE |
| 281030 | TGQVW | 329885 | IIFDV | 360107 | RAFSN | 382452 | PPASP |
| 281031 | FHPIR | 329890 | RLCCR | 360108 | QKPLY | 382453 | VIEMS |
| 281038 | HSCLL | 329896 | QRKED | 360113 | VDAEC | 382454 | FFFFG |
| 281043 | HARTC | 329902 | KNGDG | 360117 | NTSED | 382455 | KPQYF |
| 281047 | RAQSA | 329904 | EIINS | 360120 | ISAEL | 382458 | EPRSH |
| 281081 | PSPSE | 329906 | RKYHL | 360124 | PTVIT | 382459 | PSGVA |
| 281092 | KRKLT | 329908 | YMAIQ | 360129 | YSLIS | 382461 | QKKRP |
| 281129 | EEYGS | 329915 | SNIYT | 360133 | KAILN | 382463 | PEANT |
| 281131 | KETPL | 329918 | IRIFS | 360141 | QYELL | 382466 | ECLPD |
| 281133 | FAGGN | 329920 | QSSPQ | 360147 | LGWSV | 382468 | YYHRL |
| 281141 | QQEDD | 329926 | YFSKV | 360154 | GSEED | 382473 | VCMCV |
| 281142 | NLENI | 329930 | PELFS | 360157 | KWPAQ | 382475 | PEASV |
| 281146 | EKCDI | 329932 | THVQT | 360158 | IQEQL | 382477 | EHWVW |
| 281154 | DIGGR | 329933 | SNSVP | 360163 | FSAFS | 382479 | AQKAK |
| 281156 | PYGRY | 329943 | PEPGL | 360165 | QRRCF | 382480 | AGFKL |
| 281171 | NVSKS | 329956 | IAPGP | 360166 | NREHS | 382483 | LYPTH |
| 281172 | EGSSH | 329964 | MTWMH | 360167 | AGLWS | 382485 | FAIFG |
| 281182 | SLLQE | 329967 | NVDCL | 360170 | SESFE | 382486 | SEVRT |
| 281187 | NNCRQ | 329968 | VLVLG | 360171 | EIGRR | 382488 | RWRLQ |
| 281189 | LKTAN | 329970 | EGSDS | 360177 | PRGPV | 382489 | SRRLQ |
| 281228 | LNLIL | 329982 | YKRIS | 360180 | RMLKL | 382490 | ALHHD |
| 281243 | TPAYF | 329990 | TKTSI | 360181 | CLKAP | 382491 | VLSSI |
| 281273 | HRQAS | 329991 | HPEST | 360184 | NVKID | 382492 | RTPPL |
| 281282 | VASQI | 329993 | NPNIL | 360190 | CCLQQ | 382495 | TGQFR |
| 281321 | YSAGI | 329995 | RYINQ | 360195 | RSLGL | 382496 | HRARP |
| 281339 | GRSFC | 329996 | WQLVS | 360197 | GNNIP | 382507 | LQQLK |
| 281382 | TYKNN | 330005 | LPVFC | 360198 | QMTAD | 382513 | PSETS |
| 281416 | AAGGH | 330010 | TAVAS | 360209 | TMTPH | 382514 | TDKFQ |
| 281419 | HFIAD | 330027 | RGALH | 360210 | KQGVH | 382517 | TDKFQ |
| 281437 | PPPLS | 330031 | RLFYL | 360211 | HILFL | 382519 | WLSPM |
| 281441 | GSDEE | 330032 | KLSKA | 360214 | RLSLE | 382520 | QELLY |
| 281445 | PADDV | 330035 | WRGPW | 360216 | WERNA | 382521 | FYKMG |
| 281453 | LLDQG | 330049 | IPLPC | 360217 | VPKTR | 382524 | YSSVS |
| 281455 | STIKV | 330051 | EEHSD | 360222 | TQLYP | 382535 | RPLVS |
| 281456 | IKKYV | 330052 | GRIIG | 360233 | SWYLG | 382536 | RLQDQ |
| 281471 | TWTVY | 330054 | AQKAK | 360235 | ETNQL | 382540 | HCARP |
| 281474 | KPPHP | 330060 | TVQRK | 360237 | EEIKL | 382542 | YVSSL |
| 281508 | GVSPC | 330070 | APGPF | 360240 | TPATF | 382543 | PASAS |
| 281513 | AQHWV | 330072 | RRRWT | 360241 | ICLLV | 382544 | GKAST |
| 281523 | LFAPY | 330074 | AAKKK | 360244 | YLKEL | 382548 | LLESR |
| 281525 | SDESD | 330075 | SIAYC | 360247 | AVPQV | 382550 | RPAWA |
| 281527 | SSIFV | 330083 | QAHAP | 360248 | WKDMV | 382553 | ALERP |
| 281543 | TQSSK | 330085 | PQLQD | 360252 | ATTSD | 382554 | NESRS |
| 281581 | REFSR | 330087 | HRPFQ | 360262 | IMNDD | 382560 | IQETP |
| 281589 | GVPTV | 330098 | SPSPS | 360264 | MNRQG | 382569 | VITLL |
| 281620 | KSRKK | 330100 | RESLA | 360266 | QLQTF | 382572 | LEPRL |
| 281623 | SKRAR | 330101 | HSSTK | 360268 | QRNTN | 382577 | HQTTQ |
| 281631 | QTATG | 330113 | DGYYS | 360269 | KEPSL | 382579 | LTLSD |
| 281701 | ESLSR | 330138 | RISYL | 360270 | KRTGS | 382582 | PASLE |
| 281703 | GSTED | 330141 | WNSPT | 360272 | INHGS | 382584 | CQCKI |
| 281708 | DVDMK | 330146 | DPLDT | 360273 | AVLSS | 382585 | LLWAP |
| 281722 | QASFF | 330148 | HGTSI | 360275 | PQAGS | 382587 | TQLLS |
| 281741 | CSKVN | 330153 | RGALS | 360281 | TLDCS | 382589 | PYKCE |
| 281753 | FHFPP | 330158 | QRTQR | 360286 | PRNKK | 382594 | GEAVA |
| 281772 | VKKNR | 330160 | PSQLS | 360290 | TTLAR | 382596 | QTEDS |
| 281806 | LKSHF | 330161 | RRSKT | 360304 | FGGWI | 382604 | RSNRR |
| 281815 | VIFPQ | 330162 | LASCL | 360305 | TVFPK | 382606 | LSTPS |
| 281821 | RMVPV | 330165 | YPSRI | 360312 | PDTQG | 382609 | PPSVS |
| 281828 | VGPFL | 330172 | SRVAG | 360315 | ARGGS | 382612 | RSCLK |
| 281830 | IHQNS | 330180 | RDGQS | 360316 | KAARH | 382613 | DFLSN |
| 281834 | EFCVL | 330188 | GMDAV | 360317 | CFIPV | 382614 | DAAGL |
| 281871 | TRGST | 330196 | KWGNG | 360320 | SDPTP | 382616 | PQPPK |
| 281881 | DSKEI | 330199 | PESEL | 360323 | RAVSF | 382619 | LQLLG |
| 281882 | LGHRL | 330211 | RTPDE | 360324 | KELLG | 382620 | KLEKK |
| 281923 | CKDCL | 330216 | CCIHF | 360327 | PVQTV | 382621 | RMAKM |
| 281924 | YEMFE | 330218 | PLTHI | 360328 | NSKGT | 382626 | TSTEV |
| 281928 | IMNIL | 330219 | SLFSD | 360334 | NSKGT | 382629 | TSTEV |
| 281938 | EVTCT | 330221 | YLLYQ | 360337 | NSKGT | 382631 | VCIRG |
| 281950 | LTASQ | 330236 | WTMSH | 360338 | NSKGT | 382634 | HANTP |
| 281961 | RDSTV | 330240 | CRIQR | 360340 | ACGCH | 382635 | ITGTQ |
| 282003 | KATKS | 330247 | LSSAI | 360342 | NSKGT | 382642 | APHSS |
| 282018 | KETRV | 330248 | KIHTG | 360344 | NSKGT | 382645 | TKISR |
| 282020 | RGTSI | 330264 | QHIQE | 360345 | NSKGT | 382646 | KQLLA |
| 282026 | DSKRS | 330265 | NSSSF | 360350 | NSKGT | 382649 | KHRDI |
| 282030 | SEVLP | 330267 | QICLS | 360352 | NSKGT | 382650 | FGWSR |
| 282032 | NKHAG | 330271 | RGTQA | 360354 | NSKGT | 382654 | KKVEE |
| 282041 | LDHIR | 330276 | SSRLI | 360358 | NSKGT | 382655 | ITEGY |
| 282045 | SLLYQ | 330278 | GPTLL | 360359 | NSKGT | 382657 | AEVCP |
| 282050 | AGFEA | 330280 | KNAFS | 360365 | MKTYK | 382659 | ASGEK |
| 282058 | DMMEL | 330284 | CRAAA | 360366 | YLQDT | 382662 | RDIQK |
| 282059 | RKYLL | 330289 | MIQKL | 360371 | MTMSV | 382670 | KNCAG |
| 282074 | ATVYN | 330298 | TPLGG | 360372 | CFIPV | 382671 | GHELS |
| 282091 | KAKSQ | 330302 | REVYL | 360373 | YNYIA | 382673 | VQGNQ |
| 282096 | ADEEE | 330307 | KAKGK | 360376 | EESSS | 382674 | RNGMD |
| 282111 | TKSAH | 330320 | LEDNA | 360379 | EESSS | 382675 | YLLQP |
| 282141 | VVDLY | 330326 | VETFH | 360382 | QTHKK | 382678 | WEGPC |
| 282146 | TSGDR | 330330 | MRADG | 360383 | PECLF | 382679 | AQKGS |
| 282169 | YQTLL | 330338 | FTQKD | 360389 | AHSEI | 382685 | PTPSS |
| 282185 | ERNVP | 330341 | YDAPL | 360396 | GYQSV | 382692 | GSRDS |
| 282199 | IKVVD | 330342 | KFLPL | 360398 | GVSPF | 382695 | IDLQR |
| 282223 | VGYIW | 330346 | SNQII | 360401 | KTSLH | 382699 | APSAP |
| 282226 | IKVNL | 330349 | SSMDF | 360411 | ACPGP | 382700 | PKKKI |
| 282249 | RETSL | 330358 | HTGNL | 360431 | QEVKE | 382706 | PQPPK |
| 282251 | KSKNY | 330361 | RFNFF | 360437 | PAAKP | 382708 | HIAPS |
| 282272 | DENGK | 330375 | EKDKI | 360441 | TFPNV | 382709 | FFFLR |
| 282276 | KAHRT | 330376 | RNRAG | 360442 | GMNLG | 382711 | ALDKV |
| 282282 | KVHTG | 330381 | WMSNH | 360443 | EGGIG | 382713 | PVSDG |
| 282292 | TGEIQ | 330382 | ETLGA | 360444 | SEPHL | 382720 | QNAEP |
| 282308 | NVHKG | 330384 | ASRNV | 360445 | TCEAL | 382724 | SHCTW |
| 282326 | AHGHM | 330386 | PGLFP | 360455 | TFPNV | 382738 | GVNHR |
| 282333 | GRAAP | 330389 | FRLFK | 360464 | GKDDK | 382743 | DSKPY |
| 282344 | YQSRD | 330393 | CDLTV | 360471 | GNLGA | 382746 | HDLHM |
| 282356 | ILPEY | 330426 | PAAEL | 360472 | VDLSA | 382752 | DQFAA |
| 282366 | HNGLD | 330432 | NCVIS | 360473 | EADES | 382755 | WMQKC |
| 282369 | YQEDM | 330433 | GSLYI | 360475 | LTPES | 382756 | STKNT |
| 282388 | SISDD | 330442 | RTVAL | 360481 | DSLLN | 382758 | GLRTG |
| 282397 | STPPI | 330457 | ACLLF | 360483 | LEFKE | 382759 | NFCIF |
| 282406 | GPTLL | 330458 | RSKDK | 360485 | LFNRV | 382760 | HTHNE |
| 282412 | SGEKI | 330459 | MKSVQ | 360492 | LENID | 382770 | CAIPF |
| 282441 | FLTWL | 330460 | SNSQK | 360495 | QEWSY | 382771 | TASRL |
| 282466 | YIQVI | 330475 | SKTGL | 360497 | AIPIT | 382773 | HCLDL |
| 282470 | ENLLF | 330484 | AESKL | 360498 | RNFLR | 382774 | RCLLC |
| 282486 | YVTQM | 330485 | TGLLG | 360500 | QEQSD | 382775 | WPDWS |
| 282493 | HRNSS | 330488 | PDRKA | 360502 | TCLML | 382777 | CRSTC |
| 282499 | ASDLP | 330496 | CPRLA | 360515 | AFLAC | 382779 | ENEGI |
| 282507 | KVKET | 330501 | PFHSK | 360519 | ERNLL | 382780 | QRLYY |
| 282512 | VLLEQ | 330509 | DAYLE | 360525 | KIKPI | 382781 | PPVNT |
| 282516 | DGTSS | 330516 | RDRPY | 360530 | FDDVL | 382785 | LLPYP |
| 282538 | LGKDM | 330523 | EELPK | 360532 | LKSKF | 382790 | GQPNF |
| 282541 | HPEHT | 330568 | RSGST | 360538 | SIISG | 382794 | WTTSR |
| 282549 | RFQVL | 330570 | GRQIY | 360540 | EYCSK | 382795 | RLTDT |
| 282561 | DDLEI | 330572 | DIYLE | 360541 | KSIKS | 382799 | SGRGR |
| 282570 | PEKKN | 330587 | CSCCA | 360542 | YLRNN | 382804 | TDADN |
| 282572 | PPSSK | 330596 | LSPGF | 360549 | LETNV | 382807 | GAGSE |
| 282574 | GYETQ | 330606 | EEGDK | 360560 | ENSVG | 382811 | EQSHH |
| 282588 | KKMEK | 330608 | TSANL | 360561 | FWLQD | 382813 | KKLKD |
| 282605 | GVLAS | 330612 | PMGLL | 360563 | MAHGF | 382820 | GSQTV |
| 282606 | AQYID | 330625 | LCSWP | 360569 | GNAKL | 382826 | GSGCH |
| 282611 | KVDEK | 330631 | AVTLR | 360576 | TETWA | 382839 | IEEVD |
| 282635 | IIFIL | 330633 | GSLEL | 360577 | KFVGK | 382840 | DWWGN |
| 282641 | GYGTF | 330634 | IISRN | 360583 | CQMPF | 382841 | RTGGK |
| 282670 | AVYLE | 330638 | QGLAS | 360593 | PLTFG | 382842 | EALPH |
| 282673 | RCEAV | 330650 | QHTSF | 360597 | AAAQA | 382843 | SFAEA |
| 282701 | SCACR | 330658 | GYSHG | 360608 | RHRES | 382846 | CAWPL |
| 282728 | YFNAG | 330659 | VEPST | 360613 | SWRDC | 382847 | VKWMS |
| 282753 | SSSTL | 330662 | VDIAA | 360621 | ALKDE | 382849 | PGDQP |
| 282841 | KEENE | 330671 | LLLEA | 360626 | TRDNN | 382850 | KLQLL |
| 282849 | CTRKI | 330678 | QGLSM | 360635 | PRLSL | 382851 | TSASQ |
| 282878 | PNCAC | 330694 | PAPSS | 360640 | LFATT | 382853 | IKGKV |
| 282881 | ESQKQ | 330701 | RLEEL | 360642 | EPRGL | 382854 | DDWES |
| 282884 | EGIYV | 330702 | ISCKT | 360643 | TFMLL | 382861 | QEADA |
| 282892 | SLPDS | 330710 | GGCFF | 360651 | DSTFI | 382863 | EDADD |
| 282903 | SFIDP | 330714 | WGCGC | 360652 | DNQFK | 382865 | GPTRV |
| 282908 | RGCSS | 330720 | CRRTC | 360658 | KNEAN | 382866 | SNSPT |
| 282924 | KLKFM | 330730 | GRKGY | 360659 | PPALP | 382874 | ARARI |
| 282928 | NEWYV | 330732 | LLGSL | 360671 | IKKYT | 382875 | PGVLT |
| 282957 | LEHLY | 330737 | STPYK | 360672 | TIITL | 382877 | RVGLN |
| 282999 | GKKKK | 330746 | SCGFY | 360674 | ESSLP | 382883 | RSFQP |
| 283003 | IQEKI | 330753 | KENAQ | 360676 | SQSLH | 382885 | PPPVY |
| 283006 | NVDMM | 330766 | RAHLS | 360678 | AVHPR | 382886 | PPVNT |
| 283025 | LVGHT | 330768 | PMAIT | 360682 | DFDET | 382892 | LIDHF |
| 283027 | NLISS | 330770 | CCSVT | 360683 | FNSNT | 382901 | SPRKP |
| 283050 | NLKTP | 330774 | TPHPP | 360684 | TPRTP | 382903 | AQRNS |
| 283109 | SFWGE | 330785 | HQKIH | 360686 | AEITQ | 382904 | ILEKP |
| 283122 | MTGDI | 330787 | EVTWK | 360688 | LEDEL | 382908 | VQAGL |
| 283128 | RLIKA | 330805 | SSKKT | 360689 | GMVDG | 382915 | IEEVD |
| 283131 | KKLKL | 330808 | SGISP | 360690 | SRNQN | 382920 | KALLF |
| 283141 | STQTS | 330812 | LALAL | 360692 | KQRPH | 382925 | YAVVV |
| 283147 | ACGCH | 330813 | SKRGK | 360702 | SAAVD | 382942 | TLRKL |
| 283148 | RTRHS | 330836 | LRGEN | 360718 | QNCSC | 382944 | YAVVV |
| 283195 | ECGQI | 330838 | KEEAR | 360722 | HLPAS | 382945 | TPDVR |
| 283206 | SEKIM | 330842 | SRSSG | 360730 | KKASQ | 382946 | FILWP |
| 283243 | EKSDQ | 330843 | AGRPV | 360757 | NTKYS | 382949 | VKTIR |
| 283249 | LSTDC | 330862 | FFIKF | 360761 | VQPAQ | 382950 | SSDLR |
| 283254 | RENQK | 330864 | EEEEI | 360762 | RIATF | 382951 | KGKSP |
| 283256 | RESKK | 330875 | GLSSV | 360765 | PEAFK | 382956 | GLWSG |
| 283268 | RTVQS | 330877 | KGVPV | 360776 | QVNEY | 382957 | SSDLR |
| 283285 | EMETL | 330878 | GLLRV | 360777 | ICSVS | 382958 | KGKSP |
| 283290 | RHFTM | 330889 | PGHLH | 360782 | YRLFR | 382968 | DLSTN |
| 283297 | FVSNE | 330892 | RFHDL | 360793 | TKTAK | 382971 | LILSS |
| 283309 | QEFVV | 330904 | MNQEK | 360797 | DTEGS | 382973 | IYFEG |
| 283351 | IISNV | 330918 | PALQG | 360798 | EISEA | 382982 | EGEEY |
| 283357 | ELQEV | 330930 | LLQLL | 360803 | KTEPS | 382983 | DSDLE |
| 283410 | LVGVK | 330935 | KAILF | 360806 | RDQSI | 382984 | VKTIR |
| 283415 | RKKLD | 330937 | VGHDP | 360808 | VWYFS | 382985 | DSDLE |
| 283429 | DFNLL | 330940 | PQMGS | 360811 | DHAQF | 382986 | DSDLE |
| 283441 | AESED | 330945 | AKKLV | 360815 | SSVSL | 382987 | SSDLR |
| 283474 | MEAER | 330949 | FKQIS | 360816 | AQFLG | 382988 | GTLCR |
| 283478 | QSVGL | 330959 | QSYPK | 360817 | IQGMM | 382989 | RPAAF |
| 283506 | SSFTM | 330963 | TKKNY | 360821 | TLNRT | 382991 | GRSGI |
| 283507 | FPMGQ | 330965 | SSGPL | 360822 | DETQL | 382995 | SRDRA |
| 283534 | QLIDT | 330973 | ATVDG | 360825 | LNDTL | 382998 | MMTLE |
| 283550 | SQGSQ | 330994 | LIQHQ | 360828 | YKRRK | 383000 | IFVFL |
| 283627 | CCVIL | 331012 | TPYHY | 360829 | LNKPT | 383002 | DSDLE |
| 283629 | HIILK | 331020 | STLPY | 360851 | PSICD | 383006 | GSDSI |
| 283632 | KQIFF | 331039 | VGRLL | 360852 | LEAKE | 383007 | AKKIV |
| 283635 | SARYV | 331040 | TSDAK | 360858 | RSDEL | 383008 | DSDLE |
| 283645 | GMEQS | 331044 | IANNA | 360860 | LRLSI | 383015 | KMDLL |
| 283646 | EKPFC | 331047 | DALFK | 360869 | VRQGP | 383017 | IKDEI |
| 283684 | RTGDS | 331057 | PMGHM | 360872 | LALTK | 383018 | TPKKD |
| 283694 | RYTFT | 331062 | EENKL | 360874 | LNPIF | 383020 | SSNLR |
| 283713 | QLGFF | 331065 | GTTMD | 360876 | SEQLN | 383022 | DSDLE |
| 283752 | RFSSP | 331086 | KDTHV | 360878 | SRQIG | 383023 | EEKEE |
| 283871 | PAEPN | 331087 | PGAEV | 360881 | APEKQ | 383024 | LAGFY |
| 283875 | EDLFE | 331100 | GYTIW | 360886 | KAHKS | 383025 | DSDLE |
| 283882 | SKMKP | 331105 | TEAAC | 360891 | SWNGE | 383032 | VPNLQ |
| 283905 | YYFYN | 331108 | NSKLP | 360895 | MRKYQ | 383034 | SFNIL |
| 283916 | QQTGI | 331111 | EKEDS | 360899 | KYNLQ | 383035 | NSPHT |
| 283921 | NFNFS | 331127 | RERQK | 360905 | PSTPF | 383047 | APSIW |
| 283928 | RKMQQ | 331131 | LLLRP | 360910 | LQSGS | 383048 | AAFWA |
| 283930 | YTEAW | 331137 | MGNSG | 360912 | EEGIQ | 383050 | THTLP |
| 283936 | VESQM | 331152 | LSFTS | 360913 | TQESF | 383054 | TCFLR |
| 283946 | REKQP | 331167 | TCRGA | 360918 | SVPAR | 383069 | CGFNL |
| 283950 | AQPVR | 331178 | PNTLP | 360922 | VCGRC | 383075 | WRQRV |
| 283977 | PQPGF | 331186 | STEGA | 360931 | QAIHR | 383080 | FQTSA |
| 284000 | DNAGQ | 331194 | RVRMK | 360938 | VKGIY | 383081 | KMQEH |
| 284006 | VQDVL | 331199 | GHCII | 360939 | FDKEG | 383084 | PHTQT |
| 284031 | KKVDS | 331201 | LISFD | 360942 | IQSLV | 383085 | SLLDS |
| 284037 | REVSS | 331202 | LCRTP | 360944 | LPKLF | 383088 | AAFWA |
| 284049 | SSRKT | 331205 | ISVWF | 360950 | GAGPR | 383090 | SKNSQ |
| 284054 | EAPNL | 331210 | PMPGP | 360958 | DKDQL | 383092 | KKKKK |
| 284061 | IKATE | 331211 | GQKVS | 360961 | HRKST | 383093 | MLKTP |
| 284073 | TNGYH | 331214 | EHCST | 360966 | RSFLR | 383108 | HASAP |
| 284081 | RHRLV | 331218 | ETEDG | 360967 | SRKPE | 383115 | LSSAL |
| 284110 | FGWDG | 331242 | QPRSI | 360968 | KLSEC | 383126 | EESEQ |
| 284116 | HNFSA | 331258 | AREML | 360971 | DKDQL | 383129 | GGPPS |
| 284136 | RAVAT | 331267 | EGLAS | 360973 | ETFVS | 383132 | STEGA |
| 284142 | LNLEI | 331275 | ARRLR | 360985 | LSPVW | 383136 | NSKRQ |
| 284154 | QPVHL | 331276 | LCSAP | 360988 | NTITT | 383138 | YDCLL |
| 284202 | KRNEH | 331288 | RTDFS | 360991 | PGSGK | 383142 | AEALK |
| 284224 | TTPFL | 331291 | SRTLC | 360995 | PSEQL | 383143 | LEQQT |
| 284240 | DFMSL | 331294 | AMWQK | 360997 | GKEGT | 383144 | ISACT |
| 284245 | IDPEA | 331298 | MDGQR | 360998 | LRKAL | 383147 | IGAND |
| 284259 | ESKTK | 331301 | LGMAS | 360999 | LHYFK | 383154 | PQAIL |
| 284262 | INFFI | 331302 | RFSRR | 361000 | EYLQN | 383156 | EGSTE |
| 284268 | REENE | 331303 | KNDSH | 361001 | VALGI | 383158 | AGGRR |
| 284273 | TLLQE | 331305 | VVLAN | 361005 | NQNKT | 383159 | GMGTL |
| 284274 | EETSL | 331308 | NSQLM | 361007 | IQDNL | 383161 | QMRRP |
| 284287 | RGKLF | 331310 | HRKAV | 361009 | VDGDL | 383162 | QNCQI |
| 284288 | CDEHP | 331313 | LYPRA | 361010 | CSDDA | 383166 | KTRSY |
| 284292 | GPSGD | 331327 | LQLAL | 361014 | PPTAL | 383169 | GWSHC |
| 284308 | PDKVT | 331342 | KAGKM | 361021 | QITKV | 383185 | IVYWS |
| 284311 | RSVSL | 331352 | FSNAS | 361024 | DTLPN | 383186 | TRLHT |
| 284320 | KPPTL | 331358 | AEDEN | 361025 | SGGLE | 383187 | RLRAL |
| 284322 | IPGKW | 331363 | MKERR | 361027 | VEYLI | 383190 | CVCIC |
| 284368 | DAWVT | 331368 | RFSSP | 361032 | CKQPG | 383194 | AVTDV |
| 284384 | LQSAV | 331369 | SVPKR | 361036 | HCLKP | 383206 | PHWHF |
| 284395 | TRKRR | 331370 | QGEEF | 361038 | DLTGD | 383207 | KRLQS |
| 284414 | IFQDF | 331376 | LPAQL | 361042 | GTPGV | 383212 | LIENK |
| 284425 | HSDEP | 331381 | ALRKF | 361043 | QRGLR | 383213 | KLTFM |
| 284437 | LDETD | 331385 | VHALG | 361044 | GIPTK | 383214 | LGDLA |
| 284440 | LCKAA | 331397 | AGQAR | 361045 | PRPSD | 383215 | RSQLL |
| 284476 | LIKTL | 331406 | AEGRS | 361049 | SLEKN | 383216 | SRLAC |
| 284481 | SLVFQ | 331411 | SPYDF | 361054 | ISKAG | 383220 | FSTDV |
| 284483 | SMMNW | 331418 | YLNSG | 361057 | KKKEW | 383222 | LSLRS |
| 284486 | RFSLC | 331435 | MLTGG | 361064 | TSDEL | 383224 | AGITS |
| 284503 | QCQFS | 331463 | YLFYF | 361066 | DQKYC | 383225 | CVSLL |
| 284509 | KTVKL | 331465 | TVKKS | 361076 | ATKLG | 383226 | SRPAC |
| 284551 | TLAPQ | 331470 | LTGAV | 361081 | ILLQL | 383227 | VPGAT |
| 284562 | KIFRF | 331471 | FKQIS | 361083 | FGLLK | 383234 | SKERW |
| 284563 | KSNTA | 331472 | YKRIS | 361084 | AQNFV | 383235 | REVAS |
| 284601 | SVKKK | 331473 | RDFYP | 361090 | GVAVL | 383239 | RIQII |
| 284617 | YSARA | 331474 | QNGHG | 361092 | RAGLV | 383245 | FCKGI |
| 284629 | TFQRK | 331479 | DLTEL | 361095 | RPGQV | 383246 | DFGES |
| 284630 | QNHLE | 331500 | SFFPS | 361097 | LMFKT | 383247 | GFDRS |
| 284637 | FVENI | 331504 | RKRHR | 361100 | KVTML | 383249 | RQPPQ |
| 284669 | KLSKL | 331514 | HRKCF | 361101 | SPGSD | 383250 | KGQLV |
| 284674 | LPVSE | 331538 | APPSH | 361104 | CVPEH | 383251 | PLSGV |
| 284676 | LGFTW | 331540 | NHQVL | 361113 | SCLRS | 383254 | IVSYN |
| 284690 | RCTLQ | 331546 | HGGRR | 361124 | VCSFL | 383255 | DNIWL |
| 284694 | QRNAV | 331555 | LSNGS | 361125 | DKPRR | 383256 | SEAEM |
| 284719 | QPKKK | 331556 | IHKLL | 361138 | AKKID | 383263 | PQMSS |
| 284727 | ILFAM | 331557 | NIRAS | 361141 | QDPRM | 383264 | GDSPC |
| 284759 | WIQHR | 331567 | IISRN | 361151 | AVIRF | 383269 | TAQYH |
| 284770 | LYPKA | 331572 | LKKGC | 361154 | ASPGP | 383271 | HQPQG |
| 284776 | YVKRL | 331577 | VHTIK | 361158 | GKQDG | 383280 | SYGWQ |
| 284811 | NFLDC | 331581 | TVVLS | 361162 | TWGSS | 383283 | FMPDY |
| 284818 | QPNSN | 331589 | ELQEV | 361165 | GEEKK | 383284 | SSSET |
| 284856 | QAGES | 331598 | LGPLF | 361173 | GAERV | 383285 | NNLQQ |
| 284878 | DGSIV | 331600 | PYCVM | 361177 | NEYFC | 383288 | QPGPL |
| 284881 | LPINN | 331602 | HLLED | 361180 | ILDYV | 383292 | SPTAS |
| 284885 | QSFLH | 331605 | AEKSE | 361185 | TEDNS | 383293 | RLREG |
| 284894 | DDSKA | 331608 | ALSSS | 361186 | RSMVG | 383296 | NSVPV |
| 284898 | QEVRG | 331614 | RFHMS | 361189 | NKAYF | 383297 | IDFNS |
| 284920 | NWNRK | 331619 | SELNF | 361190 | WKLYH | 383298 | SGRRF |
| 284951 | RNHHG | 331620 | KPARG | 361199 | ALPQV | 383299 | RPHGL |
| 284981 | EQMQN | 331625 | SWFLG | 361200 | KQGLL | 383301 | SKPRN |
| 284984 | MAECS | 331626 | FRKTF | 361202 | PKRGR | 383303 | SYTLV |
| 284987 | LLKKC | 331636 | VATFS | 361207 | GNRKK | 383306 | ETHPL |
| 284995 | DQDDL | 331640 | HQMLK | 361213 | LRSTC | 383309 | ASLTR |
| 285013 | LYIFL | 331641 | KILGY | 361215 | SALTK | 383315 | NDIPY |
| 285018 | MYTCK | 331643 | EYHIR | 361219 | DSPVF | 383317 | ISARC |
| 285021 | PFEQL | 331647 | EIRLE | 361223 | HLLFE | 383318 | FNSRL |
| 285023 | AAGNS | 331648 | PFHKN | 361232 | TSQKV | 383319 | TTRTG |
| 285039 | FLNEV | 331649 | QLSWF | 361238 | FLNRV | 383324 | AIKER |
| 285046 | DASVL | 331664 | SIWTS | 361243 | GDPCP | 383327 | PCWPG |
| 285071 | SESRN | 331669 | IISRN | 361245 | SPWEE | 383328 | LTVSE |
| 285082 | ELVKC | 331672 | HMLRK | 361246 | KGFRE | 383329 | KRERR |
| 285083 | FEKWW | 331678 | DFLGP | 361254 | ASEKK | 383338 | ALNTV |
| 285093 | IQSTA | 331681 | SPVPP | 361266 | GPATG | 383340 | IQRRD |
| 285094 | DETGE | 331682 | NSSED | 361272 | SKSPF | 383345 | IYSHR |
| 285106 | SSADR | 331691 | KASDK | 361275 | DRSPA | 383346 | PCGLP |
| 285116 | RYVIT | 331692 | SPVQA | 361276 | IDQDL | 383347 | NKAER |
| 285141 | REKTN | 331696 | DHFSL | 361281 | EALEP | 383349 | TPETP |
| 285176 | KIVNS | 331697 | LQALI | 361286 | SSRPH | 383353 | QVRSR |
| 285199 | PESSF | 331698 | QQSNV | 361287 | RKLVF | 383358 | PGADA |
| 285208 | GGCSC | 331700 | SPDSP | 361288 | ARKGK | 383365 | TGCUG |
| 285238 | DAGLA | 331704 | SMTCS | 361298 | KKRPQ | 383366 | TVVPL |
| 285243 | SKLTY | 331710 | QRLTR | 361302 | LIRKH | 383367 | CNKSL |
| 285274 | TEEQP | 331718 | LRKEV | 361310 | KPLTH | 383371 | GALGQ |
| 285279 | GLVGC | 331719 | RDGDF | 361311 | NCVRC | 383373 | LTNWE |
| 285298 | ISSAQ | 331720 | RNRMI | 361322 | RRIYL | 383376 | RGQEF |
| 285311 | VCQKI | 331727 | GLPGK | 361325 | PVRGR | 383380 | REKLH |
| 285362 | REYFI | 331734 | CSSSK | 361327 | TSKPF | 383382 | GTDES |
| 285379 | KASFK | 331736 | PSYIL | 361328 | EILSV | 383384 | LTVSE |
| 285381 | RASFK | 331741 | DLQFP | 361336 | EAQAS | 383385 | PCWPG |
| 285383 | GLGRR | 331745 | ECGCG | 361337 | LGDGF | 383387 | AIKER |
| 285393 | YIPIL | 331746 | PQNAN | 361338 | LISAW | 383389 | HKDKS |
| 285398 | KRFRK | 331748 | HVRCC | 361345 | PPKHR | 383393 | VMSQV |
| 285402 | RKMIL | 331760 | LRPFF | 361347 | RQKQT | 383396 | SYSHI |
| 285419 | EHSFA | 331766 | VRFNI | 361359 | VQERQ | 383398 | SELYI |
| 285420 | TENCS | 331774 | VRFVL | 361366 | VVCEF | 383399 | QHESL |
| 285518 | VTIKA | 331775 | TLGRK | 361373 | PAAGP | 383402 | ASSTY |
| 285599 | HFQQQ | 331776 | IVLCM | 361377 | QHCSQ | 383407 | PDVTI |
| 285600 | NSWKN | 331784 | FGLIL | 361381 | QDSRI | 383418 | SLAPS |
| 285605 | KQEVA | 331787 | GYAVK | 361382 | TICEF | 383422 | AIKER |
| 285667 | KTNFN | 331789 | FINMY | 361383 | IASLP | 383424 | AQPPK |
| 285681 | PADGR | 331791 | DYCPR | 361390 | TICEF | 383425 | SSLMG |
| 285689 | LENGD | 331815 | KGALP | 361391 | PAVLY | 383426 | STVIL |
| 285697 | QRLIL | 331817 | RMDTS | 361392 | PQAPA | 383428 | PLDQS |
| 285718 | DDWES | 331823 | NNDIT | 361397 | RHCSQ | 383429 | RRRLQ |
| 285737 | LNSKL | 331827 | DEGSL | 361400 | TICEF | 383430 | DITLT |
| 285805 | SMASR | 331835 | MGAGP | 361404 | TICEF | 383431 | RGAKL |
| 285814 | RSSNQ | 331843 | IISRN | 361405 | QCPED | 383432 | TNRIQ |
| 285848 | HDTLG | 331845 | QSSGC | 361410 | CRRVQ | 383433 | VKSGV |
| 285850 | DPKSN | 331846 | KKETK | 361413 | VREDR | 383434 | KEPRA |
| 285871 | KPVEI | 331849 | KNKRL | 361418 | YTADY | 383435 | GRRIT |
| 285873 | QTFCM | 331857 | RGVLL | 361423 | DIVQR | 383436 | LIYSA |
| 285879 | SFSSE | 331867 | RCGST | 361433 | LEQEG | 383438 | PQSAL |
| 285894 | QTLGK | 331873 | NNENI | 361435 | KKPKE | 383442 | LAEYS |
| 285896 | NNMQQ | 331877 | KKNYV | 361446 | IPLVT | 383445 | ERKRE |
| 285900 | GATGL | 331885 | LWLLP | 361454 | RQTQI | 383447 | LLGLI |
| 285928 | PQGRR | 331894 | TFKHI | 361458 | GGSAV | 383449 | HFKTW |
| 285930 | FHEEF | 331897 | ALGRQ | 361463 | CPPEL | 383454 | ILEKH |
| 285947 | YTIVS | 331902 | CMKRT | 361465 | AVQMD | 383456 | IRKQR |
| 285949 | NGLCL | 331907 | RKQHV | 361468 | PACPL | 383457 | VRASP |
| 285968 | RISRN | 331912 | GYLYL | 361472 | PQLRG | 383458 | FSLSS |
| 285979 | CFIPV | 331915 | LSDEC | 361473 | RAIME | 383461 | THIHT |
| 286031 | IHTLG | 331921 | DRCCC | 361475 | YDGLV | 383465 | PLQFY |
| 286049 | GKSQV | 331926 | PKTNI | 361483 | WEGAV | 383474 | RSSLP |
| 286063 | KEDRN | 331927 | GQIST | 361486 | EPHPT | 383477 | QTCRD |
| 286067 | RLDAK | 331933 | DYYPR | 361508 | STAAV | 383479 | CLPAW |
| 286091 | TKEEL | 331938 | ATVQV | 361509 | KGFVG | 383485 | PLQYF |
| 286096 | SFWWS | 331939 | RELGP | 361512 | SHVPL | 383488 | HQRLY |
| 286122 | AIQIQ | 331965 | TLSLK | 361518 | AETEM | 383489 | EQPRA |
| 286143 | VSKVG | 331981 | NSTHL | 361520 | EMVYR | 383491 | KIVYL |
| 286149 | AKLLW | 331983 | EFIFV | 361522 | GNLLN | 383505 | PQTRV |
| 286175 | NPFAQ | 331987 | SAEAL | 361524 | MNRRY | 383506 | KKSSD |
| 286181 | LISQV | 332013 | KMTEF | 361536 | KVKAN | 383514 | ISIGE |
| 286186 | DALSL | 332018 | SGQRR | 361543 | VNTAF | 383515 | ISTDV |
| 286190 | KGQES | 332020 | ISIGE | 361547 | VPTVN | 383517 | RPPHS |
| 286193 | LMCGL | 332021 | QRFST | 361548 | YWQQP | 383519 | GLRGR |
| 286195 | IKRGY | 332034 | QSGWQ | 361554 | TAEEA | 383520 | KQCIS |
| 286196 | IKASS | 332036 | KEEEF | 361556 | QPDKR | 383521 | KKSSD |
| 286201 | GETAV | 332049 | SDTCF | 361557 | EISGS | 383522 | FPPGF |
| 286234 | EELEC | 332059 | GTINL | 361558 | SFALR | 383523 | MSMIK |
| 286298 | SLSSD | 332062 | REYLH | 361562 | NKQPY | 383535 | QEEMM |
| 286301 | NYQFC | 332068 | IKIFS | 361567 | GLLLG | 383536 | IPDVR |
| 286307 | VHLAQ | 332069 | AYIHM | 361568 | CREWM | 383537 | DVAHS |
| 286317 | MNERP | 332103 | GPPYL | 361569 | AIVAF | 383543 | EALRF |
| 286332 | MPRHF | 332105 | AGHPV | 361571 | EVKTN | 383544 | PDNMK |
| 286349 | LQKHF | 332110 | QHMFC | 361579 | ADSQV | 383545 | CCVIS |
| 286353 | HREGF | 332111 | PNSKF | 361581 | VLYSL | 383546 | FAGVA |
| 286355 | KSDLP | 332116 | SPVDT | 361583 | SGVKR | 383551 | LSPGS |
| 286364 | VGKAS | 332117 | KIGII | 361592 | KAPSF | 383552 | LPSLC |
| 286371 | ITARA | 332118 | LPVQV | 361596 | FCKYE | 383555 | METSP |
| 286380 | ILPGI | 332123 | ESTEI | 361597 | PGSCL | 383556 | KDWKD |
| 286398 | AHVEV | 332124 | EHSVI | 361598 | EQVQL | 383558 | AESPF |
| 286424 | AVNKK | 332130 | PSMDK | 361599 | QPPGE | 383561 | ITTGY |
| 286428 | TKNKA | 332134 | LHLCC | 361602 | ALGYQ | 383562 | NKGSK |
| 286452 | ETAAS | 332139 | DSVLA | 361603 | QGLTH | 383563 | SCSRC |
| 286479 | GSLTI | 332141 | ITRAG | 361608 | SLGGP | 383564 | PRRRR |
| 286482 | EEKGV | 332148 | RVPAW | 361612 | RRGME | 383565 | AQTRL |
| 286494 | DEDEL | 332150 | RALIL | 361616 | LAFKI | 383566 | LAGYQ |
| 286523 | KKCGR | 332151 | SQSQC | 361625 | GTKLQ | 383567 | YSQFS |
| 286544 | LVSLA | 332152 | KAYNQ | 361626 | QGGAE | 383568 | HLASE |
| 286548 | EYNLV | 332155 | AQPVG | 361628 | PENTS | 383569 | SYLGG |
| 286574 | SNYIW | 332162 | CGQAD | 361636 | MEGVQ | 383570 | FYLTP |
| 286603 | PRLES | 332163 | KSKQK | 361642 | IGDRN | 383571 | RRRWT |
| 286604 | KKKRE | 332164 | CLACS | 361646 | DHPIT | 383572 | GCCNT |
| 286614 | MQEWV | 332170 | SSPED | 361651 | ELKLK | 383573 | KKKKK |
| 286621 | KPDFH | 332171 | LVNCH | 361658 | RHMQR | 383577 | FVGSL |
| 286639 | PRFQP | 332177 | SKDDE | 361662 | CTGCL | 383578 | RLWFA |
| 286648 | FLSTL | 332184 | GTLDR | 361666 | KQVQN | 383580 | LPSQA |
| 286657 | STLER | 332185 | IGSQH | 361667 | ILLMA | 383581 | GELWK |
| 286688 | VCGKH | 332194 | KAKSK | 361668 | RWRKR | 383583 | QILLM |
| 286692 | LSRDI | 332198 | PRPVL | 361669 | ACPFF | 383585 | TAKKD |
| 286713 | HSHLG | 332204 | RDSVV | 361672 | SCPHS | 383586 | KKVTP |
| 286719 | SPGAH | 332212 | AERDA | 361681 | ELKTD | 383587 | LKVQV |
| 286732 | GLDTQ | 332215 | GLNPL | 361683 | QQQRK | 383589 | LLAMK |
| 286733 | NPSRK | 332223 | SREDL | 361699 | SINHD | 383591 | DPTVS |
| 286744 | SCQEG | 332225 | PCWQV | 361701 | SVKQQ | 383592 | PAAEA |
| 286749 | TICAQ | 332241 | LVQHW | 361705 | APPQT | 383593 | LGRDT |
| 286758 | KRKIP | 332247 | DDSVA | 361707 | RPYPI | 383594 | TLKSK |
| 286760 | GQWDG | 332256 | GDKNP | 361712 | DIPYV | 383595 | SPVQA |
| 286777 | GVEGQ | 332258 | PAAEA | 361718 | FCLMP | 383596 | GTWDF |
| 286788 | DDQND | 332274 | RCCVM | 361721 | GSVSC | 383598 | GFFST |
| 286794 | SDSTS | 332287 | QSQGY | 361725 | TAGSK | 383599 | ESPER |
| 286800 | CTTDE | 332288 | SASGS | 361726 | CMSIL | 383608 | LAGYQ |
| 286808 | STSYV | 332290 | PFTGP | 361731 | LQDSK | 383609 | AQTRL |
| 286809 | RSQYV | 332293 | WLGGY | 361740 | YPYLM | 383610 | YLRGK |
| 286824 | QYEMV | 332298 | EFRRF | 361752 | SSPAV | 383612 | SRHRR |
| 286827 | LNTEI | 332304 | ITALG | 361759 | FCLMP | 383614 | FHLSY |
| 286835 | AEAPR | 332313 | TVQFW | 361761 | VTPNF | 383615 | PAEPN |
| 286878 | EKCRE | 332325 | ALGYQ | 361763 | QNINI | 383617 | VEVKF |
| 286897 | EQERL | 332326 | LIKVG | 361765 | DVPYV | 383619 | HLASE |
| 286918 | LTGKG | 332340 | LPQAT | 361768 | SPSCS | 383620 | FPPGF |
| 286953 | APGHH | 332353 | GSSSN | 361775 | KRLIG | 383621 | VVQYH |
| 287008 | REIYL | 332359 | DIPYV | 361776 | FCLIP | 383624 | FVDIM |
| 287020 | SCGCR | 332368 | PSQRA | 361777 | EGEDD | 383626 | GRPDS |
| 287022 | EWAKK | 332369 | VTTYA | 361784 | QKIHT | 383629 | GSIPT |
| 287025 | FLKTL | 332371 | GTAQD | 361787 | ETGRF | 383631 | AVSWQ |
| 287038 | QTGEK | 332379 | SCALL | 361790 | FRNKV | 383632 | EEWVL |
| 287042 | NDSLR | 332389 | YYLPD | 361794 | KMLPS | 383634 | EVSYA |
| 287078 | PRSKL | 332407 | VILSL | 361795 | PGACS | 383635 | LKMLS |
| 287097 | MELWL | 332413 | EESPK | 361800 | AEVAA | 383639 | LCKTR |
| 287139 | ECGCL | 332415 | DTELE | 361803 | PQMWK | 383641 | WKLWR |
| 287143 | FVCSF | 332419 | KQSQC | 361811 | SRVYK | 383643 | KLNTQ |
| 287152 | CLGSN | 332424 | SEIRR | 361813 | FCLMP | 383644 | PAAGI |
| 287156 | VDRPS | 332433 | ITVLQ | 361818 | NEFYA | 383646 | EQRGR |
| 287169 | YEISV | 332437 | KSRLP | 361833 | DVEPN | 383652 | PAAGI |
| 287196 | QVYTN | 332443 | RWQQW | 361834 | SIKGP | 383653 | DNDIM |
| 287202 | ANRPY | 332444 | STGAS | 361845 | TMKQH | 383655 | SPSYT |
| 287239 | SYMRR | 332445 | KKEEC | 361850 | NGLAL | 383656 | SFLMS |
| 287254 | VLDNL | 332448 | LDPDT | 361852 | FTSPI | 383657 | DDTAL |
| 287272 | RQNGL | 332449 | SHTFK | 361855 | NPIST | 383662 | KDVIL |
| 287275 | PKKYC | 332452 | VWPYK | 361857 | CVGSS | 383668 | QGKSM |
| 287295 | NIHED | 332455 | GTFNF | 361859 | GVSGE | 383670 | GTFNF |
| 287322 | EKKGL | 332468 | DLPDP | 361865 | IPFLG | 383671 | SQDGA |
| 287364 | LSQGS | 332473 | KRRRV | 361867 | NPLFT | 383672 | TDTSV |
| 287380 | HLLAA | 332482 | LASCL | 361868 | SILGF | 383673 | AVLSD |
| 287387 | GSKNC | 332488 | TQAPL | 361871 | RCPQK | 383674 | IFQDF |
| 287394 | FSCSR | 332495 | LCNHD | 361882 | RSPPA | 383675 | RPAKV |
| 287396 | NLFKF | 332498 | RSGME | 361892 | RQQNF | 383676 | ENCCL |
| 287437 | HRHSE | 332500 | FIIRN | 361894 | LKRTD | 383678 | HSTEV |
| 287450 | HCNLF | 332502 | DMPRT | 361895 | LASIL | 383680 | ISVGL |
| 287461 | RGNAH | 332504 | LSWDN | 361900 | KQNLH | 383681 | DKEGA |
| 287474 | LGSCL | 332511 | KLPLK | 361914 | QQVAP | 383682 | EECIR |
| 287482 | QLPNS | 332513 | EDEDS | 361915 | ACGCH | 383683 | RARAP |
| 287490 | GYEHP | 332516 | QTNEL | 361917 | VLTKL | 383684 | SAVHL |
| 287497 | GAEPQ | 332528 | VYVLS | 361918 | ESHTI | 383685 | NSFRY |
| 287538 | NEWYV | 332529 | KVTVH | 361920 | KTAMA | 383690 | IISDF |
| 287543 | ELKCE | 332530 | LKNYI | 361921 | SSICF | 383694 | TFQRA |
| 287546 | PVTED | 332536 | SISGA | 361923 | KPYLS | 383696 | EDRRQ |
| 287585 | HSQGP | 332549 | IESDV | 361926 | PPDEL | 383706 | LVFVF |
| 287590 | KLQVL | 332560 | LTICP | 361927 | QSNLA | 383722 | KSREP |
| 287598 | ALLFQ | 332565 | FDFYL | 361929 | PRDEL | 383723 | NCLPP |
| 287600 | RLFYV | 332579 | HPITF | 361932 | KRQHL | 383724 | PRPRF |
| 287641 | TFTSC | 332591 | EATNV | 361935 | CDMQK | 383726 | SRYQK |
| 287647 | FPFPP | 332592 | VRTWS | 361940 | QVIQF | 383727 | GDTLW |
| 287652 | RLYSL | 332595 | EKLQT | 361942 | CGERS | 383729 | NKHWY |
| 287653 | LPLQS | 332596 | TFLLC | 361943 | EIGAF | 383730 | KDAFS |
| 287660 | ENFLN | 332600 | LANCT | 361944 | TLHGG | 383732 | LLPER |
| 287667 | KTRRT | 332601 | KKIMF | 361949 | AAATS | 383733 | RSHIG |
| 287675 | IRKPS | 332602 | SGQRR | 361952 | RWKSI | 383736 | MKKHQ |
| 287706 | SSPGS | 332603 | QAVVC | 361957 | HTVGP | 383738 | SHHAQ |
| 287709 | RIVTS | 332604 | SQTAY | 361965 | AGQNL | 383739 | KLALE |
| 287713 | INASG | 332613 | DPYPG | 361970 | LVLNS | 383743 | FTGEY |
| 287727 | LENIV | 332615 | TRSSP | 361982 | RGAGQ | 383750 | PTPPP |
| 287748 | DGCKL | 332624 | YAPEL | 361987 | KEKRG | 383754 | WLPFL |
| 287766 | KEAYI | 332626 | DCGLL | 361990 | DWQHL | 383756 | ILLIK |
| 287773 | EPSPP | 332643 | SVSED | 361993 | ESLSD | 383757 | LICTW |
| 287777 | CLTKM | 332646 | TLKAE | 361998 | RFLKK | 383759 | TGMSH |
| 287814 | DIVQP | 332655 | VVDLT | 361999 | RMWWV | 383760 | RTQPG |
| 287820 | YKDLY | 332656 | LSEVA | 362001 | DSPSE | 383762 | VAKQI |
| 287859 | HYFSD | 332659 | LHDAL | 362013 | GDAEL | 383763 | GFCGG |
| 287878 | ETETE | 332668 | GPTIL | 362014 | PPFDL | 383765 | PTWTT |
| 287899 | QRDSR | 332670 | ELKSM | 362021 | KVKVS | 383766 | ALGSG |
| 287907 | FSRQH | 332674 | CLDKC | 362026 | LLFTR | 383767 | ALPLS |
| 287908 | RVTVM | 332679 | SFLPR | 362028 | HKAGL | 383768 | AVATT |
| 287916 | VSHTT | 332690 | SGYYY | 362029 | TRLKT | 383769 | PASAS |
| 287934 | GETTV | 332695 | LKSPA | 362033 | IYTHT | 383770 | LLLFI |
| 287936 | TKKTA | 332698 | DPTVS | 362034 | DSYKK | 383771 | TGVSH |
| 287957 | VANHL | 332706 | EGEED | 362036 | EATET | 383772 | ANIVS |
| 287968 | VIFPQ | 332713 | PSTHT | 362041 | KKEKK | 383776 | DGPVP |
| 287996 | VLHKV | 332721 | FTLQS | 362044 | KACGQ | 383779 | DYRHK |
| 288014 | DHCLG | 332722 | RVKQL | 362054 | VLWRS | 383783 | ISACT |
| 288022 | MKVND | 332723 | IKKKK | 362056 | AEAAG | 383787 | PQPSE |
| 288025 | KKPRC | 332724 | QQFAF | 362057 | NKENF | 383788 | PQPPE |
| 288040 | WGPGS | 332727 | IPKKK | 362058 | GEPRP | 383790 | AQEDP |
| 288048 | PFPHF | 332729 | FPPGF | 362063 | STGAP | 383793 | EDTEM |
| 288050 | DLHGK | 332737 | SEAEC | 362066 | TSCMS | 383796 | EDTEM |
| 288063 | AAAQP | 332738 | GSGCH | 362070 | KPTKN | 383797 | AQRDK |
| 288065 | FKNTE | 332740 | AWPPA | 362071 | VHLLT | 383798 | AQEDP |
| 288071 | EKIAN | 332744 | ISWAT | 362073 | KPDTF | 383799 | SFTED |
| 288087 | SPFEA | 332750 | ESISV | 362082 | TAIHL | 383803 | SAGGA |
| 288098 | EGLLP | 332756 | ESNVL | 362085 | SDASP | 383811 | SVCFL |
| 288111 | YTSKF | 332766 | DCLAA | 362089 | QCIDG | 383816 | YRHPP |
| 288135 | VHDDV | 332769 | SEDVI | 362092 | PRNAI | 383822 | PCLLK |
| 288139 | CITTL | 332771 | PPAPK | 362094 | LRPGQ | 383825 | SYFLF |
| 288167 | GCCSL | 332772 | LSERE | 362095 | RDICE | 383831 | SRLTA |
| 288177 | TPFYW | 332773 | AHSEQ | 362100 | PQTFS | 383832 | RTHTG |
| 288197 | MLFSR | 332777 | HQGHQ | 362103 | PGSVQ | 383835 | YRVHL |
| 288207 | LIGRS | 332788 | RSTDV | 362105 | SRRKR | 383836 | LPGAI |
| 288228 | PETPM | 332790 | PGKNK | 362107 | SQAGP | 383837 | KSKSL |
| 288235 | YVTKI | 332791 | VRRCN | 362110 | RQGLL | 383838 | IPLNL |
| 288266 | RESEA | 332805 | PFALY | 362111 | QYEIV | 383845 | LLPHP |
| 288309 | NESKV | 332806 | PGQAN | 362114 | LCNPL | 383851 | QKELA |
| 288350 | RKIII | 332809 | QEEVS | 362116 | FATAA | 383852 | VDGCR |
| 288390 | FPRQS | 332812 | SSTSS | 362121 | FQNNF | 383853 | ALAQG |
| 288400 | TIKLS | 332816 | HPPAE | 362122 | MLGRV | 383855 | GLPKC |
| 288422 | MPRYT | 332818 | EQVLM | 362123 | EKEEL | 383856 | TAQSS |
| 288425 | LRRWW | 332823 | LGEKV | 362125 | KDIVL | 383857 | ECPAS |
| 288462 | SSGAE | 332827 | DKLLL | 362127 | SSFSK | 383858 | TAKKD |
| 288490 | LETAV | 332834 | PDSWM | 362131 | FKSAD | 383859 | PVFKT |
| 288532 | SGFKL | 332838 | KGLML | 362134 | PVAGL | 383864 | LEGSL |
| 288548 | YIQSG | 332840 | AAADN | 362135 | HASYL | 383869 | KGNLI |
| 288561 | PRKSQ | 332845 | RRFSV | 362136 | EQWLM | 383870 | EDKDD |
| 288602 | AFPVH | 332848 | PPVLP | 362139 | QGVQV | 383872 | LDQLQ |
| 288607 | VCQVW | 332855 | LLGTV | 362144 | IGEKD | 383873 | GCWLQ |
| 288634 | TEEEL | 332861 | RVHVQ | 362153 | FRQKQ | 383876 | FEMES |
| 288666 | KQSSG | 332874 | AQKAK | 362160 | GVQSR | 383878 | SHHAQ |
| 288680 | RFLEV | 332875 | FEFRA | 362170 | DSIIQ | | |
| 288699 | SSGIW | 332879 | EHCHT | 362171 | KKYRK | | |
| 288710 | RVPTK | 332886 | CSSFD | 362172 | LLLLP | | |

In some embodiments, the PDZbm used in the anchoring domain of the CAR of the present disclosure may be ESIV (SEQ ID NO: 1), or HPMRCMNYITKLYSEAKTKRKENVQHSKLEEKHIQVPESIV (SEQ ID NO: 3), or a functional variant thereof.

In some embodiments, the PDZbm comprises the amino acid sequence set forth in SEQ ID NO: 1 or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 1. In certain embodiments, the nucleotide sequence that encodes the PDZbm comprises the nucleotide sequence that encodes the amino acid sequence of SEQ ID NO: 1, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 1. In certain embodiments, the nucleotide sequence that encodes the PDZbm comprises the nucleotide sequence set forth in SEQ ID NO: 2, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 2. In certain embodiments, the PDZbm comprises the amino acid sequence set forth in SEQ ID NO: 1. In certain embodiments, the nucleotide sequence that encodes the PDZbm comprises the nucleotide sequence set forth in SEQ ID NO: 2.

In some embodiments, the PDZbm comprises the amino acid sequence set forth in SEQ ID NO: 3 or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 3. In certain embodiments, the nucleotide sequence that encodes the PDZbm comprises the nucleotide sequence that encodes the amino acid sequence of SEQ ID NO: 3, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 3. In certain embodiments, the nucleotide sequence that encodes the PDZbm comprises the nucleotide sequence set forth in SEQ ID NO: 4, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 4. In certain embodiments, the PDZbm comprises the amino acid sequence set forth in SEQ ID NO: 3. In certain embodiments, the nucleotide sequence that encodes the PDZbm comprises the nucleotide sequence set forth in SEQ ID NO: 4.

### Additional Genes

In addition to the CAR construct, the CAR may further comprise at least one additional gene that encodes an additional peptide. Examples of additional genes can include a transduced host cell selection marker, an in vivo tracking marker, cellular marker, epitope tag, a cytokine, a suicide gene, safety switch, or some other functional gene. In certain embodiments, the functional additional gene can induce the expression of another molecule. In certain embodiments, the functional additional gene can increase the safety of the CAR. For example, the CAR construct may comprise an additional gene which is truncated CD19 (tCD19). The tCD19 can be used as a tag. Expression of tCD19 may also help determine transduction efficiency.

Other examples of additional genes include genes that encode polypeptides with a biological function; examples include, but are not limited to, cytokines, chimeric cytokine receptors, dominant negative receptors, safety switches (CD20, truncated EGFR or HER2, inducible caspase 9 molecules). As another example, the CAR construct may comprise an additional gene which is a synNotch receptor. Once activated, the synNotch receptor can induce the expression of a target gene (e.g., a second CAR and/or bispecific molecule).

In some embodiments, the CAR may comprise one or more additional nucleotide sequences encoding one or more additional polypeptide sequences. As a non-limiting example, the one or more additional polypeptide sequences may be selected from one or more cellular markers, epitope tags, cytokines, safety switches, dimerization moieties, or degradation moieties.

In certain embodiments, the CAR comprises at least one additional gene (i.e., a second gene). In certain embodiments, the CAR comprises one second gene. In other embodiments, the CAR comprises two additional genes (i.e., a third gene). In yet another embodiment, the CAR comprises three additional genes (i.e., a fourth gene). In certain embodiments, the additional genes are separated from each other and the CAR construct. For example, they may be separated by 2A sequences and/or an internal ribosomal entry sites (IRES). In certain examples, the CAR can be at any position of the polynucleotide chain (for example construct A: CAR, second gene, third gene, fourth gene; construct B: second gene, CAR, third gene, fourth gene; etc.)

Non-limiting examples of classes of additional genes that can be used to increase the effector function of CAR containing host cells, include (a) secretable cytokines (e.g., but not limited to, IL-7, IL-12, IL-15, IL-18), (b) membrane bound cytokines (e.g., but not limited to, IL-15), (c) chimeric cytokine receptors (e.g., but not limited to, IL-2/IL-7, IL-4/IL-7), (d) constitutive active cytokine receptors (e.g., but not limited to, C7R), (e) dominant negative receptors (DNR; e.g., but not limited to TGFRII DNR), (f) ligands of costimulatory molecules (e.g., but not limited to, CD80, 4-1BBL), (g) nuclear factor of activated T-cells (NFATs) (e.g., NFATc1, NFATc2, NFATc3, NFATc4, and NFAT5), (h) antibodies, including fragments thereof and bispecific antibodies (e.g., but not limited to, bispecific T-cell engagers (BiTEs)), or (i) a second CAR.

In some embodiments, the additional gene sequence may be derived from tCD19. In some embodiments, the tCD19 sequence comprises the amino acid sequence set forth in SEQ ID NO: 77 or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 77. In certain embodiments, the nucleotide sequence encoding the tCD19 sequence comprises the nucleotide sequence that encodes the amino acid sequence of SEQ ID NO: 77, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 77. In certain embodiments, the nucleotide sequence encoding the tCD19 sequence comprises the sequence set forth in SEQ ID NO: 78 or 79, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 78 or 79. In certain embodiments, the tCD19 sequence comprises the amino acid sequence of SEQ ID NO: 33. In certain embodiments, the nucleotide sequence encoding the tCD19 sequence comprises the nucleotide sequence set forth in SEQ ID NO: 78 or 79.

In certain embodiments, the additional gene may be regulated by an NFAT dependent-promoter. Activation of the T-cell or other lymphocyte leads to activation of the transcription factor NFAT resulting in the induction of the expression of the protein encoded by the gene linked with the NFAT dependent promoter. One or more members of the NFAT family (i.e., NFATc1, NFATc2, NFATc3, NFATc4, and NFAT5) is expressed in most cells of the immune system. NFAT-dependent promoters and enhancers tend to have three to five NFAT binding sites.

In certain embodiments, the functional additional gene can be a suicide gene. A suicide gene is a recombinant gene that will cause the host cell that the gene is expressed in to undergo programmed cell death or antibody mediated clearance at a desired time. Suicide genes can function to increase the safety of the CAR. In another embodiment, the additional gene is an inducible suicide gene. Non-limiting examples of suicide genes include i) molecules that are expressed on the cell surface and can be targeted with a clinical grade monoclonal antibody including CD20, EGFR or a fragment thereof, HER2 or a fragment thereof, and ii) inducible suicide genes (e.g., but not limited to inducible caspase 9 (see Straathof et al. (2005) Blood. 105 (11): 4247-4254; US Publ. No. 2011/0286980, each of which are incorporated herein by reference in their entirety for all purposes)).

In certain aspects, CARs of the present disclosure may be regulated by a safety switch. As used herein, the term “safety switch” refers to any mechanism that is capable of removing or inhibiting the effect of a CAR from a system (e.g., a culture or a subject). Safety switches can function to increase the safety of the CAR.

The function of the safety switch may be inducible. Non-limiting examples of safety switches include (a) molecules that are expressed on the cell surface and can be targeted with a clinical grade monoclonal antibody including CD20, EGFR or a fragment thereof, HER2 or a fragment thereof, and (b) inducible suicide genes (e.g., but not limited to herpes simplex virus thymidine kinase (HSV-TK) and inducible caspase 9 (see Straathof et al. (2005) Blood. 105 (11): 4247-4254; US Publ. No. 2011/0286980, each of which are incorporated herein by reference in their entirety for all purposes).

In some embodiments, the safety switch is a CD20 polypeptide. Expression of human CD20 on the cell surface presents an attractive strategy for a safety switch. The inventors and others have shown that cells that express CD20 can be rapidly eliminated with the FDA approved monoclonal antibody rituximab through complement-mediated cytotoxicity and antibody-dependent cell-mediated cytotoxicity (see e.g., Griffioen, M., et al. Haematologica 94, 1316-1320 (2009), which is incorporated herein by reference in its entirety for all purposes). Rituximab is an anti-CD20 monoclonal antibody that has been FDA approved for Chronic Lymphocytic Leukemia (CLL) and Non-Hodgkin's Lymphoma (NHL), among others (Storz, U. MAbs 6, 820-837 (2014), which is incorporated herein by reference in its entirety for all purposes). The CD20 safety switch is non-immunogenic and can function as a reporter/selection marker in addition to a safety switch (Bonifant, C. L., et al. Mol Ther 24, 1615-1626 (2016); van Loenen, M. M., et al. Gene Ther 20, 861-867 (2013); each of which is incorporated herein by reference in its entirety for all purposes).

In some embodiments, the polynucleotide sequence(s) encoding the CARs of the present disclosure may be expressed in an inducible fashion, for example, as may be achieved with an inducible promoter, an inducible expression system, an artificial signaling circuits, and/or drug-induced splicing.

In some embodiments, the polynucleotide sequence(s) encoding the CARs of the present disclosure may be expressed in an inducible fashion, such as that which may be achieved with i) an inducible promoter, for example, but not limited to promotors that may be activated by T cell activation (e.g. NFAT, Nur66, IFNg) or hypoxia; ii) an inducible expression system, for example, but not limited to doxycycline- or tamoxifen-inducible expression system; iii) artificial signaling circuits including, but not limited to, SynNotch, and/or iv) drug-induced splicing. By way of a non-limiting example, drug-induced splicing methods and/or compositions useful in the practice of the present disclosure may be based those described in, for example, Monteys et al., 2021 [39], the contents of which is incorporated herein by reference in its entirety for all purposes.

In some embodiments, the polynucleotide sequence(s) encoding the CARs disclosed herein may be expressed as a ‘split molecule’ in which for example, transmembrane and intracellular signaling regions, or any other domains or regions of the CAR, may be assembled only in the presence of a heterodimerizing small molecule (e.g., small organic molecule, nucleic acid, polypeptide, or a fragment, isoform, variant, analog, or derivative thereof), as described in, for example, Wu et al., 2015 [40], the contents of which is incorporated herein by reference in its entirety for all purposes.

In some embodiments, the polynucleotide sequence(s) encoding the CARs herein may further encode a moiety so that the stability of CAR may be regulated with a small molecule, including but not limited to, the “SWIFF” technology or an immunomodulatory drug (IMiD)-inducible degron as described, for example, in Juillerat et al., 2019 [41], Carbonneau et al., 2021 [42], and Jan et al., 2021 [43], the contents of each of which is incorporated herein by reference in its entirety for all purposes.

In some embodiments, the sequence encoding an additional gene is operably linked to the sequence encoding CAR via a sequence encoding a self-cleaving peptide and/or an Internal Ribosome Entry Site (IRES) as disclosed herein.

Non-limiting examples of self-cleaving peptide sequences includes Thoseaasigna virus 2A (T2A; AEGRGSLLTCGDVEENPGP, SEQ ID NO: 66, EGRGSLLTCGDVEENPGP, SEQ ID NO: 64, or GSGEGRGSLLTCGDVEENPGP, SEQ ID NO: 67); the foot and mouth disease virus (FMDV) 2A sequence (F2A; GSGSRVTELLYRMKRAETYCPRPLLAIHPTEARHKQKIVAPVKQLLNFDLLKLAGDV ESNPGP, SEQ ID NO: 68), Sponge (Amphimedon queenslandica) 2A sequence (LLCFLLLLLSGDVELNPGP, SEQ ID NO: 69; or HHFMFLLLLLAGDIELNPGP, SEQ ID NO: 70); acorn worm 2A sequence (Saccoglossus kowalevskii) (WFLVLLSFILSGDIEVNPGP, SEQ ID NO: 71); amphioxus (Branchiostoma floridae) 2A sequence (KNCAMYMLLLSGDVETNPGP, SEQ ID NO: 72; or MVISQLMLKLAGDVEENPGP, SEQ ID NO: 73); porcine teschovirus-1 2A sequence (P2A; GSGATNFSLLKQAGDVEENPGP, SEQ ID NO: 74); and equine rhinitis A virus 2A sequence (E2A; GSGQCTNYALLKLAGDVESNPGP, SEQ ID NO: 75). In some embodiments, the separation sequence is a naturally occurring or synthetic sequence. In certain embodiments, the separation sequence includes the 2A consensus sequence D-X-E-X-NPGP (SEQ ID NO: 76), in which X is any amino acid residue.

Alternatively, an Internal Ribosome Entry Site (IRES) may be used to link the CAR and the additional gene. IRES is an RNA element that allows for translation initiation in a cap-independent manner. IRES can link two coding sequences in one bicistronic vector and allow the translation of both proteins in cells.

In some embodiments, the self-cleaving 2A peptide is a T2A peptide and comprises the amino acid sequence set forth in SEQ ID NO: 64. In some embodiments, the sequence encoding the T2A peptide comprises the nucleotide sequence SEQ ID NO: 65.

In certain embodiments, the host cells can be genetically modified to express not only CARs as disclosed herein but to also express fusion protein with signaling activity (e.g., costimulation, T-cell activation). These fusion proteins can improve host cell activation and/or responsiveness. In certain embodiments, the fusion protein can enhance the host cell's response to the target antigen. In certain embodiments, the fusion protein can impart resistance to suppression signals.

In certain embodiments, fusion proteins can comprise portions of CD4, CD8α, CD28, portions of a T-cell receptor, or an antigen-binding moiety (e.g., scFv) linked to a MyD88, CD40, and/or other signaling molecules.

In certain embodiments, the fusion protein comprises an extracellular target-binding domain (as disclosed above), a transmembrane domain (as described above) and a cytoplasmic domain, wherein the cytoplasmic domain comprises at least one co-stimulatory protein (as described above). In certain embodiments, the co-stimulatory fusion protein does not comprise a lymphocyte activation domain (e.g., CD3ζ). In certain embodiments, the at least one co-stimulatory protein can be a MyD88 polypeptide or functional fragment thereof, and/or a CD40 cytoplasmic polypeptide region or a functional fragment thereof.

In certain embodiments, the fusion protein comprises an extracellular domain (such as, but not limited to CD19, CD34), a transmembrane domain (as described above) and a cytoplasmic domain, wherein the cytoplasmic domain comprises at least one co-stimulatory protein (as described above). In certain embodiments, the fusion protein does not comprise a lymphocyte activation domain (e.g., CD3ζ). In certain embodiments, the at least one portion of the fusion protein can be a MyD88 polypeptide or functional fragment thereof, and/or a CD40 cytoplasmic polypeptide region or a functional fragment thereof.

Non-limiting examples of fusion proteins include, but are not limited to, the constructs in the publication of WO2019222579 and WO2016073875, which are incorporated herein by reference in its entirety for all purposes.

In certain embodiments, the fusion proteins are introduced into the host cell on a separate vector from the CAR. In certain embodiments, the fusion proteins are introduced into the host cell on the same vector as the CAR. In certain embodiments, the fusion proteins are introduced into the host cell on the same vector as the CAR but separated by a separation sequence such as 2A.

### Non-Limiting Examples of CARS

In certain embodiments, a PDZ CAR of the disclosure comprises an extracellular binding domain comprising the amino acid sequence of SEQ ID NO: 82, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 82. In certain embodiments, the extracellular binding domain of a PDZ CAR of the disclosure is encoded by a nucleotide sequence that encodes the amino acid sequence of SEQ ID NO: 82, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 82. In certain embodiments, the nucleotide sequence that encodes the extracellular binding domain of a PDZ CAR of the disclosure comprises the nucleotide sequence set forth in SEQ ID NO: 83, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 83. In certain embodiments, a PDZ CAR of the disclosure comprises an extracellular binding domain comprising the amino acid sequence set forth in SEQ ID NO: 82. In certain embodiments, the nucleotide sequence that encodes the extracellular binding domain of a PDZ CAR of the disclosure comprises the nucleotide sequence set forth in SEQ ID NO: 82.

In certain embodiments, a PDZ CAR of the disclosure comprises a cytoplasmic domain comprising the amino acid sequence of SEQ ID NO: 86, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 86. In certain embodiments, the cytoplasmic domain of a PDZ CAR of the disclosure is encoded by a nucleotide sequence that encodes the amino acid sequence of SEQ ID NO: 86, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 86. In certain embodiments, the nucleotide sequence that encodes the cytoplasmic domain of a PDZ CAR of the disclosure comprises the nucleotide sequence set forth in SEQ ID NO: 87, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 87. In certain embodiments, a PDZ CAR of the disclosure comprises a cytoplasmic domain comprising the amino acid sequence set forth in SEQ ID NO: 86. In certain embodiments, the nucleotide sequence that encodes the cytoplasmic domain of a PDZ CAR of the disclosure comprises the nucleotide sequence set forth in SEQ ID NO: 87.

In certain embodiments, a PDZ CAR of the disclosure comprises the amino acid sequence of SEQ ID NO: 92, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 92. In certain embodiments, a PDZ CAR of the disclosure is encoded by a nucleotide sequence that encodes the amino acid sequence of SEQ ID NO: 92, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 92. In certain embodiments, the nucleotide sequence that encodes a PDZ CAR of the disclosure comprises the nucleotide sequence set forth in SEQ ID NO: 93, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 93. In certain embodiments, a PDZ CAR of the disclosure comprises an amino acid sequence set forth in SEQ ID NO: 92. In certain embodiments, the nucleotide sequence that encodes a PDZ CAR of the disclosure comprises the nucleotide sequence set forth in SEQ ID NO: 93.

In certain embodiments, a PDZ CAR of the disclosure comprises an extracellular binding domain comprising the amino acid sequence of SEQ ID NO: 123, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 123. In certain embodiments, the extracellular binding domain of a PDZ CAR of the disclosure is encoded by a nucleotide sequence that encodes the amino acid sequence of SEQ ID NO: 123, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 123. In certain embodiments, the nucleotide sequence that encodes the extracellular binding domain of a PDZ CAR of the disclosure comprises the nucleotide sequence set forth in SEQ ID NO: 124, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 124. In certain embodiments, a PDZ CAR of the disclosure comprises an extracellular binding domain comprising the amino acid sequence set forth in SEQ ID NO: 123. In certain embodiments, the nucleotide sequence that encodes the extracellular binding domain of a PDZ CAR of the disclosure comprises the nucleotide sequence set forth in SEQ ID NO: 124.

In certain embodiments, a PDZ CAR of the disclosure comprises a cytoplasmic domain comprising the amino acid sequence of SEQ ID NO: 125, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 125. In certain embodiments, the cytoplasmic domain of a PDZ CAR of the disclosure is encoded by a nucleotide sequence that encodes the amino acid sequence of SEQ ID NO: 125, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 125. In certain embodiments, the nucleotide sequence that encodes the cytoplasmic domain of a PDZ CAR of the disclosure comprises the nucleotide sequence set forth in SEQ ID NO: 126, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 126. In certain embodiments, a PDZ CAR of the disclosure comprises a cytoplasmic domain comprising the amino acid sequence set forth in SEQ ID NO: 125. In certain embodiments, the nucleotide sequence that encodes the cytoplasmic domain of a PDZ CAR of the disclosure comprises the nucleotide sequence set forth in SEQ ID NO: 126.

In certain embodiments, a PDZ CAR of the disclosure comprises the amino acid sequence of SEQ ID NO: 115, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 115. In certain embodiments, a PDZ CAR of the disclosure is encoded by a nucleotide sequence that encodes the amino acid sequence of SEQ ID NO: 115, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 115. In certain embodiments, the nucleotide sequence that encodes a PDZ CAR of the disclosure comprises the nucleotide sequence set forth in SEQ ID NO: 116, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 116. In certain embodiments, a PDZ CAR of the disclosure comprises an amino acid sequence set forth in SEQ ID NO: 115. In certain embodiments, the nucleotide sequence that encodes a PDZ CAR of the disclosure comprises the nucleotide sequence set forth in SEQ ID NO: 116.

### Vectors

The present disclosure provides recombinant vectors comprising a polynucleotide encoding a CAR comprising polynucleotides encoding the proteins disclosed above. In certain embodiments, the polynucleotide is operatively linked to at least one regulatory element for expression of the chimeric antigen receptor.

In certain embodiments, recombinant vectors of the disclosure comprise the nucleotide sequence of SEQ ID NO: 93, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 93. In certain embodiments, recombinant vectors comprise a nucleotide sequence that encodes the amino acid sequence of SEQ ID NO: 92, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 92.

In certain embodiments, recombinant vectors of the disclosure comprise the nucleotide sequence of SEQ ID NO: 116, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 116. In certain embodiments, recombinant vectors comprise a nucleotide sequence that encodes the amino acid sequence of SEQ ID NO: 115, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 115.

In certain embodiments, the recombinant vector comprises a polynucleotide encoding a CAR, wherein the polynucleotide is operatively linked to at least one additional gene. In some embodiments, the additional gene is a tCD19.

In certain embodiments, the vector is a viral vector. In certain embodiments, the viral vector can be, but is not limited to, a retroviral vector, an adenoviral vector, an adeno-associated virus vector, an alphaviral vector, a herpes virus vector, and a vaccinia virus vector. In some embodiments, the viral vector is a lentiviral vector.

In some embodiments, the vector is a non-viral vector. In some embodiments, the viral vector may be a plasmid or a transposon (such as a PiggyBac- or a Sleeping Beauty transposon).

In some embodiments, the non-viral vector may be a minicircle plasmid. In some embodiments, the non-viral vector may be a single or double stranded DNA molecule that is used as a template for homology directed repair (HDR) based gene editing.

In certain embodiments, the polynucleotide encoding the CAR is operably linked to at least a regulatory element. The regulatory element can be capable of mediating expression of the CAR in the host cell. Regulatory elements include, but are not limited to, promoters, enhancers, initiation sites, polyadenylation (polyA) tails, IRES elements, response elements, and termination signals. In certain embodiments, the regulatory element regulates CAR expression. In certain embodiments, the regulatory element increased the expression of the CAR. In certain embodiments, the regulatory element increased the expression of the CAR once the host cell is activated. In certain embodiments, the regulatory element decreases expression of the CAR. In certain embodiments, the regulatory element decreases expression of the CAR once the host cell is activated.

In some embodiments, the promoter is an inducible promoter. Non-limiting examples of an inducible promoter are lac, sp6, T7, and Hsp70- and Hsp90-derived promoters. In some embodiments, the inducible promoter is a tetracycline (Tc)-inducible promoter.

In some embodiments, the promoter may be a T cell-specific promoter or an NK cell-specific promoter.

### CAR-Modified Host Cells

In one aspect, the present disclosure provides an isolated host cell comprising a polynucleotide or a recombinant vector described herein. In one aspect, the present disclosure provides an isolated host cell comprising a CAR described herein. In some embodiments the CAR may comprise: (a) an extracellular domain, (b) a transmembrane domain, and (c) a cytoplasmic domain comprising a signaling domain and an anchoring domain which binds to a cell polarity protein.

In some embodiments, the extracellular domain comprises an antigen-binding moiety, wherein the antigen-binding moiety may comprise, for example, an antibody or an antibody fragment. In some embodiments, the antigen-binding moiety may comprise a single chain variable fragment (scFv) such as, but not limited to, an EphA2 scFv or a B7-H3 scFv. In some embodiments, the antigen-binding moiety may comprise a ligand or peptide sequence. In some embodiments, the antigen-binding moiety may comprise a VH sequence, a VL sequence, and/or CDRs disclosed herein. In some embodiments, the antigen-binding moiety may comprise an scFv derived from an antibody or antibody fragment that binds to an antigen target disclosed herein. In some embodiments, the antigen-binding moiety may comprise an antigen-binding moiety derived from a CAR that binds to an antigen target. In some embodiments, the antigen-binding moiety may bind to a tumor antigen, antigen of extracellular matrix, antigen present on cells within the tumor microenvironment, tissue-specific antigen, autoimmune antigen or infectious antigen disclosed herein.

In some embodiments, the cell polarity protein may comprise a PDZ domain. In some embodiments, the anchoring domain may comprise a PDZ binding motif (PDZbm). In some embodiments, the PDZbm binds to, for example, Scribble. As a non-limiting example, the PDZbm may be derived from Cytotoxic and Regulatory T cell Associated Molecule (CRTAM).

In a further aspect, the present disclosure provides an isolated host cell comprising two or more polynucleotides or recombinant vectors described herein. In a further aspect, the present disclosure provides an isolated host cell comprising two or more CARs described herein.

In various embodiments, the host cell may be an allogeneic cell. In various embodiments, the host cell may be an autologous cell. In some embodiments, the host cell may be derived from a blood, marrow, tissue, or a tumor sample. In some embodiments, the host cell may be derived from an induced pluripotent stem cell (iPSC).

In various embodiments, the host cell is an immune cell. In some embodiments, when the host cell is an immune cell, the immune cell may be derived from, for example, an induced pluripotent stem cell (IPS) cell. The immune cell may be a T-cell, a natural killer (NK) cell or a macrophage.

In various embodiments, the host cell is a T-cell. T-cells may include, but are not limited to, thymocytes, naive T lymphocytes, immature T lymphocytes, mature T lymphocytes, resting T lymphocytes, or activated T lymphocytes. A T-cell can be a T helper (Th) cell, for example a T helper 1 (Th1) or a T helper 2 (Th2) cell. The T-cell can be a helper T-cell (HTL; CD4+ T-cell) CD4+ T-cell, a cytotoxic T-cell (CTL; CD8+ T-cell), a tumor infiltrating cytotoxic T-cell (TIL; CD8+ T-cell), CD4+CD8+ T-cell, or any other subset of T-cells. Other illustrative populations of T-cells suitable for use in particular embodiments include naive T-cells memory T-cells, and NKT cells.

In some embodiments, the T-cell is selected from a CD8+ T-cell, a CD4+ T-cell, a cytotoxic T-cell, an αβ T-cell receptor (TCR) T-cell, an invariant natural killer T (INKT) cell, a γδ T-cell, a memory T-cell, a memory stem T-cell (TSCM), a naïve T-cell, an effector T-cell, a T-helper cell, or a regulatory T-cell (Treg).

In various embodiments, the host cell is a NK cell. NK cell refers to a differentiated lymphocyte with a CD3− CD16+, CD3− CD56+, CD16+ CD56+ and/or CD57+ TCR-phenotype.

In some embodiments, when the host cell is a NK cell, the NK cell may be derived from peripheral, cord blood, induced pluripotent stem cells (IPSCs), and/or a cell line, for example, but not limited to NK-92 cells

In various embodiments, the host cell has been activated and/or expanded ex vivo.

In various embodiments, the host cell is an allogeneic cell. In various embodiments, the host cell is an autologous cell.

In some embodiments, the host cell is isolated from a subject having a tumor. In some embodiments, the tumor can be found within, but not limited to, breast tissue, prostate tissue, bladder tissue, oral and/or dental tissue, head and/or neck tissue, colorectal tissue, lung tissue, brain tissue, skin, lymph nodes, and bone. In some embodiments, the tumor is a cancer. In some embodiments, the cancer can be, but not limited to, breast cancer, prostate cancer, bladder cancer, oral squamous cell carcinoma, head and/or neck squamous cell carcinoma, colorectal cancer, lung cancer, brain tumors, melanoma, bone, pediatric solid tumors and brain tumors, and/or lymphoma.

In certain embodiments, the host cell is isolated from a subject having a tumor, wherein one or more cells of the tumor cells express a tumor antigen disclosed herein. Non-limiting examples of tumor cells that express a tumor antigen include acute lymphoblastic leukemia, acute myeloid leukemia, adult solid tumors and brain tumors, adrenal gland tumors, anal cancer, bile duct cancer, bladder cancer, blood cancers, bone cancer, bowel cancer, brain tumors, breast cancer, cancer of unknown primary, cancer spread to bone, cancer spread to brain, cancer spread to liver, cancer spread to lung, carcinoid, cervical cancer, children's cancers, colorectal cancer, chronic lymphocytic leukemia, chronic myeloid leukemia, colorectal cancer, ear cancer, endometrial cancer, eye cancer, follicular dendritic cell sarcoma, gallbladder cancer, gastric cancer, gastro esophageal junction cancers, germ cell tumors, gestational trophoblastic disease, glioma, glioblastoma, gynecological cancer, hairy cell leukemia, head and neck squamous cell carcinoma, high grade gliomas, Hodgkin lymphoma, Kaposi's sarcoma, kidney cancer, large bowel and rectal neuroendocrine tumors, laryngeal cancer, leukemia, Linitis plastica of the stomach, liver cancer, low grade gliomas, lung cancer, lung neuroendocrine tumors (NETs), lymphoma, malignant schwannoma, mediastinal germ cell tumors, melanoma, men's cancer, merkel cell skin cancer, mesothelioma, molar pregnancy, mouth and oropharyngeal cancer, myeloma, nasal and paranasal sinus cancer, nasopharyngeal cancer, neuroblastoma, neuroendocrine tumors, neuroendocrine tumors of the pancreas, non-Hodgkin lymphoma, non-Hodgkin lymphoma in children, esophageal cancer, oral squamous cell carcinoma, ovarian cancer, pancreatic cancer, pediatric solid tumors and brain tumors, penile cancer, persistent trophoblastic disease and choriocarcinoma, pheochromocytoma, prostate cancer, pseudomyxoma peritonei, rare cancers, rectal cancer, renal cancer, retinoblastoma, salivary gland cancer, secondary cancer, signet cell cancer, skin cancer, small bowel cancer, small bowel neuroendocrine tumors, soft tissue sarcoma, stomach cancer, stomach neuroendocrine tumors, testis cancer, thymus gland tumors, thyroid cancer, tongue cancer, tonsil cancer, tumors of the adrenal gland, unknown primary cancer, urothelial, uterine cancer, vaginal cancer, vulval cancer, Wilms' tumor, and womb cancer.

Additional non-limiting examples of tumor cells that express a tumor antigen include glioblastoma, high grade gliomas, low grade gliomas, head and neck cancers, liver cancers, lung cancers, melanoma, ovarian cancer, pancreatic cancer, prostate cancer, renal cancer, urothelial cancer, carcinoid, cervical cancers, colorectal cancer, endometrial cancer, lymphoma, skin cancer, stomach cancer, testis cancer, thyroid cancer and urothelial cancer.

In some embodiments, the host cell is derived from a blood, marrow, tissue, or a tumor sample.

In one aspect, the present disclosure provides a method of generating an isolated host cell described herein. The method includes genetically modifying the host cell with a polynucleotide encoding a CAR and optionally an additional gene (e.g., tCD19). In some embodiments, the isolated host cell may be genetically modified, for example, to enhance its function by expressing additional genes (e.g., transcription factors (e.g., c-Jun) or cytokines (e.g., IL-15) or deleting inhibitory genes (e.g., REGNASE-1, CISH, DNMT3A) with gene editing technologies, including but not limited to CRISPR-Cas9, base editors, or transcription activator-like effector nucleases (TALENs). The genetically modifying step may be conducted in vivo or ex vivo. Suitable methods of genetic modification of immune cells to knock out inhibitory genes such as REGNASE-1, and DNMT3A include those described in, e.g., PCT patent applications WO2020/219682, WO2020/210365, which are incorporated herein by reference in their entireties.

In some embodiments, the genetically modifying step is conducted ex vivo. In some embodiments, the genetic modifying step may be conducted via a viral gene delivery. In some embodiments, the genetic modifying step may be conducted via a non-viral gene delivery. The method may further include activation and/or expansion of the host cell ex vivo before, after and/or during the genetic modification.

### Isolation/Enrichment

The host cells may be autologous/autogeneic (“self”) or non-autologous (“non-self,” e.g., allogeneic, syngeneic or xenogeneic). In certain embodiments, the host cells are obtained from a mammalian subject. In other embodiments, the host cells are obtained from a primate subject. In certain embodiments, the host cells are obtained from a human subject.

Lymphocytes can be obtained from sources such as, but not limited to, peripheral blood mononuclear cells, bone marrow, lymph nodes tissue, cord blood, thymus issue, tissue from a site of infection, ascites, pleural effusion, spleen tissue, and tumors. Lymphocytes may also be generated by differentiation of stem cells. In certain embodiments, lymphocytes can be obtained from blood collected from a subject using techniques generally known to the skilled person, such as sedimentation, e.g., FICOLL™ separation.

In certain embodiments, cells from the circulating blood of a subject are obtained by apheresis. An apheresis device typically contains lymphocytes, including T-cells, monocytes, granulocytes, B cells, other nucleated white blood cells, red blood cells, and platelets. In certain embodiments, the cells collected by apheresis may be washed to remove the plasma fraction and to place the cells in an appropriate buffer or media for subsequent processing. The cells can be washed with PBS or with another suitable solution that lacks calcium, magnesium, and most, if not all other, divalent cations. A washing step may be accomplished by methods known to those in the art, such as, but not limited to, using a semiautomated flowthrough centrifuge (e.g., Cobe 2991 cell processor, or the Baxter CytoMate). After washing, the cells may be resuspended in a variety of biocompatible buffers, cell culture medias, or other saline solution with or without buffer.

In certain embodiments, host cells can be isolated from peripheral blood mononuclear cells (PBMCs) by lysing the red blood cells and depleting the monocytes. As an example, the cells can be sorted by centrifugation through a PERCOLL™ gradient. In certain embodiments, after isolation of PBMC, both cytotoxic and helper T lymphocytes can be sorted into naive, memory, and effector T-cell subpopulations either before or after activation, expansion, and/or genetic modification.

In certain embodiments, T lymphocytes can be enriched. For example, a specific subpopulation of T lymphocytes, expressing one or more markers such as, but not limited to, CD3, CD4, CD8, CD14, CD15, CD16, CD19, CD27, CD28, CD34, CD36, CD45RA, CD45RO, CD56, CD62, CD62L, CD122, CD123, CD127, CD235a, CCR7, HLA-DR or a combination thereof using either positive or negative selection techniques. In certain embodiments, the T lymphocytes for use in the compositions of the disclosure do not express or do not substantially express one or more of the following markers: CD57, CD244, CD160, PD-1, CTLA4, TIM3, and LAG3.

In certain embodiments, NK cells can be enriched. For example, a specific subpopulation of T lymphocytes, expressing one or more markers such as, but not limited to, CD2, CD16, CD56, CD57, CD94, CD122 or a combination thereof using either positive or negative selection techniques.

### Stimulation/Activation

In order to reach sufficient therapeutic doses of host cell compositions, host cells are often subjected to one or more rounds of stimulation/activation. In certain embodiments, a method of producing host cells for administration to a subject comprises stimulating the host cells to become activated in the presence of one or more stimulatory signals or agents (e.g., compound, small molecule, e.g., small organic molecule, nucleic acid, polypeptide, or a fragment, isoform, variant, analog, or derivative thereof). In certain embodiments, a method of producing host cells for administration to a subject comprises stimulating the host cells to become activated and to proliferate in the presence of one or more stimulatory signals or agents.

Host cells (e.g., T lymphocytes and NK cells) can be activated by inducing a change in their biologic state by which the cells express activation markers, produce cytokines, proliferate and/or become cytotoxic to target cells. All these changes can be produced by primary stimulatory signals. Co-stimulatory signals amplify the magnitude of the primary signals and suppress cell death following initial stimulation resulting in a more durable activation state and thus a higher cytotoxic capacity.

T cells can be activated generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety.

In certain embodiments, the T-cell based host cells can be activated by binding to an agent that activates CD3ζ.

In other embodiments, a CD2-binding agent may be used to provide a primary stimulation signal to the T-cells. For example, and not by limitation, CD2 agents include, but are not limited to, CD2 ligands and anti-CD2 antibodies, e.g., the Tl 1.3 antibody in combination with the Tl 1.1 or Tl 1.2 antibody (Meuer, S. C. et al. (1984) Cell 36:897-906) and the 9.6 antibody (which recognizes the same epitope as TI 1.1) in combination with the 9-1 antibody (Yang, S. Y. et al. (1986) J. Immunol. 137:1097-1100). Other antibodies which bind to the same epitopes as any of the above-described antibodies can also be used.

In certain embodiments, the host cells are activated by administering phorbol myristate acetate (PMA) and ionomycine. In certain embodiments, the host cells are activated by administering an appropriate antigen that induces activation and then expansion. In certain embodiments, PMA, ionomycin, and/or appropriate antigen are administered with CD3 induce activation and/or expansion.

In general, the activating agents used in the present disclosure includes, but is not limited to, an antibody, a fragment thereof and a proteinaceous binding molecule with antibody-like functions. Examples of (recombinant) antibody fragments are Fab fragments, Fv fragments, single-chain Fv fragments (scFv), a divalent antibody fragment such as an (Fab) 2′-fragment, diabodies, triabodies (Iliades, P., et al., FEBS Lett (1997) 409, 437-441), decabodies (Stone, E., et al., Journal of Immunological Methods (2007) 318, 88-94) and other domain antibodies (Holt, L. J., et al., Trends Biotechnol. (2003), 21, 11, 484-490). The divalent antibody fragment may be an (Fab) 2′-fragment, or a divalent single-chain Fv fragment while the monovalent antibody fragment may be selected from the group consisting of a Fab fragment, a Fv fragment, and a single-chain Fv fragment (scFv).

In certain embodiments, one or more binding sites of the CD3ζ agents may be a bivalent proteinaceous artificial binding molecule such as a dimeric lipocalin mutein (i.e., duocalin). In certain embodiments the receptor binding reagent may have a single second binding site, (i.e., monovalent). Examples of monovalent agents include, but are not limited to, a monovalent antibody fragment, a proteinaceous binding molecule with antibody-like binding properties or an MHC molecule. Examples of monovalent antibody fragments include, but are not limited to a Fab fragment, a Fv fragment, and a single-chain Fv fragment (scFv), including a divalent single-chain Fv fragment.

The agent that specifically binds CD3 includes, but is not limited to, an anti-CD3-antibody, a divalent antibody fragment of an anti-CD3 antibody, a monovalent antibody fragment of an anti-CD3-antibody, and a proteinaceous CD3-binding molecule with antibody-like binding properties. A proteinaceous CD3-binding molecule with antibody-like binding properties can be an aptamer, a mutein based on a polypeptide of the lipocalin family, a glubody, a protein based on the ankyrin scaffold, a protein based on the crystalline scaffold, an adnectin, and an avimer. It also can be coupled to a bead.

In certain embodiments, the activating agent (e.g., CD3-binding agents) can be present in a concentration of about 0.1 to about 10 μg/ml. In certain embodiments, the activating agent (e.g., CD3-binding agents) can be present in a concentration of about 0.2 μg/ml to about 9 μg/ml, about 0.3 μg/ml to about 8 μg/ml, about 0.4 μg/ml to about 7 μg/ml, about 0.5 μg/ml to about 6 μg/ml, about 0.6 μg/ml to about 5 μg/ml, about 0.7 μg/ml to about 4 μg/ml, about 0.8 μg/ml to about 3 μg/ml, or about 0.9 μg/ml to about 2 μg/ml. In certain embodiments, the activating agent (e.g., CD3-binding agents) is administered at a concentration of about 0.1 μg/ml, about 0.2 μg/ml, about 0.3 μg/ml, about 0.4 μg/ml, about 0.5 μg/ml, about 0.6 μg/ml, about 0.7 μg/ml, about 0.8 μM, about 0.9 μg/ml, about 1 μg/ml, about 2 μg/ml, about 3 μg/ml, about 4 μM, about 5 μg/ml, about 6 μg/ml, about 7 μg/ml, about 8 μg/ml, about 9 μg/ml, or about 10 μg/ml. In certain embodiments, the CD3-binding agents can be present in a concentration of 1 μg/ml.

NK cells can be activated generally using methods as described, for example, in U.S. Pat. Nos. 7,803,376, 6,949,520, 6,693,086, 8,834,900, 9,404,083, 9,464,274, 7,435,596, 8,026,097, 8,877,182; U.S. Patent Applications US2004/0058445, US2007/0160578, US2013/0011376, US2015/0118207, US2015/0037887; and PCT Patent Application WO2016/122147, each of which is incorporated herein by reference in its entirety.

In certain embodiments, the NK based host cells can be activated by, for example and not limitation, inhibition of inhibitory receptors on NK cells (e.g., KIR2DL1, KIR2DL2/3, KIR2DL4, KIR2DL5A, KIR2DL5B, KIR3DL1, KIR3DL2, KIR3DL3, LILRB1, NKG2A, NKG2C, NKG2E or LILRB5 receptor).

In certain embodiments, the NK based host cells can be activated by, for example and not limitation, feeder cells (e.g., native K562 cells or K562 cells that are genetically modified to express 4-1BBL and cytokines such as IL15 or IL21).

In other embodiments, interferons or macrophage-derived cytokines can be used to activate NK cells. For example and not limitation, such interferons include but are not limited to interferon alpha and interferon gamma, and such cytokines include but are not limited to IL-15, IL-2, IL-21.

In certain embodiments, the NK activating agent can be present in a concentration of about 0.1 to about 10 μg/ml. In certain embodiments, the NK activating agent can be present in a concentration of about 0.2 μg/ml to about 9 μg/ml, about 0.3 μg/ml to about 8 μg/ml, about 0.4 μg/ml to about 7 μg/ml, about 0.5 μg/ml to about 6 μg/ml, about 0.6 μg/ml to about 5 μg/ml, about 0.7 μg/ml to about 4 μg/ml, about 0.8 μg/ml to about 3 μg/ml, or about 0.9 μg/ml to about 2 μg/ml. In certain embodiments, the NK activating agent is administered at a concentration of about 0.1 μg/ml, about 0.2 μg/ml, about 0.3 μg/ml, about 0.4 μg/ml, about 0.5 μg/ml, about 0.6 μg/ml, about 0.7 μg/ml, about 0.8 μM, about 0.9 μg/ml, about 1 μg/ml, about 2 μg/ml, about 3 μg/ml, about 4 μM, about 5 μg/ml, about 6 μg/ml, about 7 μg/ml, about 8 μg/ml, about 9 μg/ml, or about 10 μg/ml. In certain embodiments, the NK activating agent can be present in a concentration of 1 μg/ml.

In certain embodiments, the activating agent is attached to a solid support such as, but not limited to, a bead, an absorbent polymer present in culture plate or well or other matrices such as, but not limited to, Sepharose or glass; may be expressed (such as in native or recombinant forms) on cell surface of natural or recombinant cell line by means known to those skilled in the art.

### Polynucleotide Transfer

In certain embodiments, the host cells are genetically modified to express a CAR described above. The host cells can be genetically modified after stimulation/activation. In certain embodiments, the host cells are modified within 12 hours, 16 hours, 24 hours, 36 hours, or 48 hours of stimulation/activation. In certain embodiments, the cells are modified within 16 to 24 hours after stimulation/activation. In certain embodiments, the host cells are modified within 24 hours.

In order to genetically modify the host cell to express the CAR, the CAR polynucleotide construct must be transferred into the host cell. Polynucleotide transfer may be via viral or non-viral gene methods. Suitable methods for polynucleotide delivery for use with the current methods include any method known by those of skill in the art, by which a polynucleotide can be introduced into an organelle, cell, tissue or organism.

In some embodiments, polynucleotides are transferred to the cell in a non-viral vector. In some embodiments, the non-viral vector is a transposon. Exemplary transposons that can be used in the present disclosure include, but are not limited to, a sleeping beauty transposon and a PiggyBac transposon.

Nucleic acid vaccines can be used to transfer CAR polynucleotides into the host cells. Such vaccines include, but are not limited to non-viral polynucleotide vectors, “naked” DNA and RNA, and viral vectors. Methods of genetically modifying cells with these vaccines, and for optimizing the expression of genes included in these vaccines are known to those of skill in the art.

In certain embodiments, the host cells can be genetically modified by methods ordinarily used by one of skill in the art. In certain embodiments, the host cells can be transduced via retroviral transduction. References describing retroviral transduction of genes are Anderson et al., U.S. Pat. No. 5,399,346; Mann et al., Cell 33:153 (1983); Temin et al., U.S. Pat. No. 4,650,764; Temin et al., U.S. Pat. No. 4,980,289; Markowitz et al., J. Virol. 62:1120 (1988); Temin et al., U.S. Pat. No. 5,124,263; International Patent Publication No. WO 95/07358, published Mar. 16, 1995, by Dougherty et al.; and Kuo et al., Blood 82:845 (1993), each of which is incorporated herein by reference in its entirety.

One method of genetic modification includes ex vivo modification. Various methods are available for transfecting cells and tissues removed from a subject via ex vivo modification. For example, retroviral gene transfer in vitro can be used to genetically modified cells removed from the subject and the cell transferred back into the subject. See e.g., Wilson et al., Science, 244:1344-1346, 1989 and Nabel et al., Science, 244 (4910): 1342-1344, 1989, both of which are incorporated herein by reference in their entity. In certain embodiments, the host cells may be removed from the subject and transfected ex vivo using the polynucleotides (e.g., expression vectors) of the disclosure. In certain embodiments, the host cells obtained from the subject can be transfected or transduced with the polynucleotides (e.g., expression vectors) of the disclosure and then administered back to the subject.

Another method of gene transfer includes injection. In certain embodiments, a cell or a polynucleotide or viral vector may be delivered to a cell, tissue, or organism via one or more injections (e.g., a needle injection). Non-limiting methods of injection include injection of a composition (e.g., a saline based composition). Polynucleotides can also be introduced by direct microinjection. Non-limiting sites of injection include, subcutaneous, intradermal, intramuscular, intranodal (allows for direct delivery of antigen to lymphoid tissues). intravenous, intraprostatic, intratumor, intralymphatic (allows direct administration of DCs) and intraperitoneal. It is understood that proper site of injection preparation is necessary (e.g., shaving of the site of injection to observe proper needle placement).

Electroporation is another method of polynucleotide delivery. See e.g., Potter et al., (1984) Proc. Nat'l Acad. Sci. USA, 81, 7161-7165 and Tur-Kaspa et al., (1986) Mol. Cell Biol., 6, 716-718, both of which are incorporated herein in their entirety for all purposes. Electroporation involves the exposure of a suspension of cells and DNA to a high-voltage electric discharge. In certain embodiments, cell wall-degrading enzymes, such as pectin-degrading enzymes, can be employed to render the host cells more susceptible to genetic modification by electroporation than untreated cells. See e.g., U.S. Pat. No. 5,384,253, incorporated herein by reference in its entirety for all purposes.

In vivo electroporation involves a basic injection technique in which a vector is injected intradermally in a subject. Electrodes then apply electrical pulses to the intradermal site causing the cells localized there (e.g., resident dermal dendritic cells), to take up the vector. These tumor antigen-expressing dendritic cells activated by local inflammation can then migrate to lymph-nodes.

Methods of electroporation for use with this disclosure include, for example, Sardesai, N. Y., and Weiner, D. B., Current Opinion in Immunotherapy 23:421-9 (2011) and Ferraro, B. et al., Human Vaccines 7:120-127 (2011), both of which are hereby incorporated by reference herein in their entirety for all purposes.

Additional methods of polynucleotide transfer include liposome-mediated transfection (e.g., polynucleotide entrapped in a lipid complex suspended in an excess of aqueous solution. See e.g., Ghosh and Bachhawat, (1991) In: Liver Diseases, Targeted Diagnosis and Therapy Using Specific Receptors and Ligands. pp. 87-104). Also contemplated is a polynucleotide complexed with Lipofectamine, or Superfect); DEAE-dextran (e.g., a polynucleotide is delivered into a cell using DEAE-dextran followed by polyethylene glycol. See e.g., Gopal, T. V., Mol Cell Biol. 1985 May; 5 (5): 1188-90); calcium phosphate (e.g., polynucleotide is introduced to the cells using calcium phosphate precipitation. See e.g., Graham and van der Eb, (1973) Virology, 52, 456-467; Chen and Okayama, Mol. Cell Biol., 7 (8): 2745-2752, 1987), and Rippe et al., Mol. Cell Biol., 10:689-695, 1990); sonication loading (introduction of a polynucleotide by direct sonic loading. See e.g., Fechheimer et al., (1987) Proc. Nat'l Acad. Sci. USA, 84, 8463-8467); microprojectile bombardment (e.g., one or more particles may be coated with at least one polynucleotide and delivered into cells by a propelling force. See e.g., U.S. Pat. Nos. 5,550,318; 5,538,880; 5,610,042; and PCT Application WO 94/09699; Klein et al., (1987) Nature, 327, 70-73, Yang et al., (1990) Proc. Nat'l Acad. Sci. USA, 87, 9568-9572); and receptor-mediated transfection (e.g., selective uptake of macromolecules by receptor-mediated endocytosis that will be occurring in a target cell using cell type-specific distribution of various receptors. See e.g., Wu and Wu, (1987) J. Biol. Chem., 262, 4429-4432; Wagner et al., Proc. Natl. Acad. Sci. USA, 87 (9): 3410-3414, 1990; Perales et al., Proc. Natl. Acad. Sci. USA, 91:4086-4090, 1994; Myers, EPO 0273085; Wu and Wu, Adv. Drug Delivery Rev., 12:159-167, 1993; Nicolau et al., (1987) Methods Enzymol., 149, 157-176), each reference cited here is incorporated by reference in their entirety for all purposes.

In further embodiments, host cells are genetically modified using gene editing with homology-directed repair (HDR). Homology-directed repair (HDR) is a mechanism used by cells to repair double strand DNA breaks. In HDR, a donor polynucleotide with homology to the site of the double strand DNA break is used as a template to repair the cleaved DNA sequence, resulting in the transfer of genetic information from the donor polynucleotide to the DNA. As such, new nucleic acid material may be inserted or copied into a target DNA cleavage site. Double strand DNA breaks in host cells may be induced by a site-specific nuclease. The term “site-specific nuclease” as used herein refers to a nuclease capable of specifically recognizing and cleaving a nucleic acid (DNA or RNA) sequence. Suitable site-specific nucleases for use in the present disclosure include, but are not limited to, RNA-guided endonuclease (e.g., CRISPR-associated (Cas) proteins), zinc finger nuclease, a TALEN nuclease, or mega-TALEN nuclease. For example, a site-specific nuclease (e.g., a Cas9+ guide RNA) capable of inducing a double strand break in a target DNA sequence is introduced to a host cell, along with a donor polynucleotide encoding a CAR of the present disclosure and optionally an additional protein (e.g., tCD19).

### Expansion/Proliferation

After the host cells are activated and transduced, the cells are cultured to proliferate. T-cells may be cultured for at least 1, 2, 3, 4, 5, 6, or 7 days, at least 2 weeks, at least 1, 2, 3, 4, 5, or 6 months or more with 1, 2, 3, 4, 5, 6, 7, 8, 9, or 10 or more rounds of expansion.

Agents that can be used for the expansion of T-cells can include interleukins, such as IL-2, IL-7, IL-15, or IL-21 (see for example Cornish et al. 2006, Blood. 108 (2): 600-8, Bazdar and Sieg, 2007, Journal of Virology, 2007, 81 (22): 12670-12674, Battalia et al, 2013, Immunology, 139 (1): 109-120). Other illustrative examples for agents that may be used for the expansion of T-cells are agents that bind to CD8, CD45 or CD90, such as αCD8, αCD45 or αCD90 antibodies. Illustrative examples of T-cell population including antigen-specific T-cells, T helper cells, cytotoxic T-cells, memory T-cell (an illustrative example of memory T-cells are CD62L|CD8| specific central memory T-cells) or regulatory T-cells (an illustrative example of Treg are CD4+CD25+CD45RA+ Treg cells).

Additional agents that can be used to expand T lymphocytes includes methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety.

In certain embodiments, the agent(s) used for expansion (e.g., IL-2) are administered at about 20 units/ml to about 200 units/ml. In certain embodiments, the agent(s) used for expansion (e.g., IL-2) are administered at about 25 units/ml to about 190 units/ml, about 30 units/ml to about 180 units/ml, about 35 units/ml to about 170 units/ml, about 40 units/ml to about 160 units/ml, about 45 units/ml to about 150 units/ml, about 50 units/ml to about 140 units/ml, about 55 units/ml to about 130 units/ml, about 60 units/ml to about 120 units/ml, about 65 units/ml to about 110 units/ml, about 70 units/ml to about 100 units/ml, about 75 units/ml to about 95 units/ml, or about 80 units/ml to about 90 units/ml. In certain embodiments, the agent(s) used for expansion (e.g., IL-2) are administered at about 20 units/ml, about 25 units/ml, about 30 units/ml, 35 units/ml, 40 units/ml, 45 units/ml, about 50 units/ml, about 55 units/ml, about 60 units/ml, about 65 units/ml, about 70 units/ml, about 75 units/ml, about 80 units/ml, about 85 units/ml, about 90 units/ml, about 95 units/ml, about 100 units/ml, about 105 units/ml, about 110 units/ml, about 115 units/ml, about 120 units/ml, about 125 units/ml, about 130 units/ml, about 135 units/ml, about 140 units/ml, about 145 units/ml, about 150 units/ml, about 155 units/ml, about 160 units/ml, about 165 units/ml, about 170 units/ml, about 175 units/ml, about 180 units/ml, about 185 units/ml, about 190 units/ml, about 195 units/ml, or about 200 units/ml. In certain embodiments, the agent(s) used for expansion (e.g., IL-2) are administered at about 5 mg/ml to about 10 ng/ml. In certain embodiments, the agent(s) used for expansion (e.g., IL-2) are administered at about 5.5 ng/ml to about 9.5 ng/ml, about 6 ng/ml to about 9 ng/ml, about 6.5 ng/ml to about 8.5 ng/ml, or about 7 ng/ml to about 8 ng/ml. In certain embodiments, the agent(s) used for expansion (e.g., IL-2) are administered at about 5 ng/ml, 6 ng/ml, 7 ng/ml, 8 ng/ml, 9 ng/ml, or 10 ng/ml.

After the host cells are activated and transduced, the cells are cultured to proliferate. NK cells may be cultured for at least 1, 2, 3, 4, 5, 6, or 7 days, at least 2 weeks, at least 1, 2, 3, 4, 5, or 6 months or more with 1, 2, 3, 4, 5, 6, 7, 8, 9, or 10 or more rounds of expansion.

Agents that can be used for the expansion of natural killer cells can include agents that bind to CD16 or CD56, such as for example αCD16 or αCD56 antibodies. In certain embodiments, the binding agent includes antibodies (see for example Hoshino et al, Blood. 1991 Dec. 15; 78 (12): 3232-40). Other agents that may be used for expansion of NK cells may be IL-15 (see for example Vitale et al. 2002. The Anatomical Record. 266:87-92, which is hereby incorporated by reference in its entirety for all purposes).

Conditions appropriate for T-cell culture include an appropriate media (e.g., Minimal Essential Media (MEM), RPMI Media 1640, Lonza RPMI 1640, Advanced RPMI, Clicks, AIM-V, DMEM, a-MEM, F-12, TexMACS, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion).

Examples of other additives for host cell expansion include, but are not limited to, surfactant, piasmanate, pH buffers such as HEPES, and reducing agents such as N-acetyl-cysteine and 2-mercaptoethanol, Antibiotics (e.g., penicillin and streptomycin), are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

In certain embodiments, host cells of the present disclosure may be modified such that the expression of an endogenous TCR, MHC molecule, or other immunogenic molecule is decreased or eliminated. When allogeneic cells are used, rejection of the therapeutic cells may be a concern as it may cause serious complications such as the graft-versus-host disease (GvHD). Although not wishing to be bound by theory, immunogenic molecules (e.g., endogenous TCRs and/or MHC molecules) are typically expressed on the cell surface and are involved in self vs non-self discrimination. Decreasing or eliminating the expression of such molecules may reduce or eliminate the ability of the therapeutic cells to cause GvHD.

In certain embodiments, expression of an endogenous TCR in the host cells is decreased or eliminated. In a particular embodiment, expression of an endogenous TCR (e.g., αβ TCR) in the host cells is decreased or eliminated. Expression of the endogenous TCR may be decreased or eliminated by disrupting the TRAC locus, TCR beta constant locus, and/or CD3 locus. In certain embodiments, expression of an endogenous TCR may be decreased or eliminated by disrupting one or more of the TRAC, TRBC1, TRBC2, CD3E, CD3G, and/or CD3D locus.

In certain embodiments, expression of one or more endogenous MHC molecules in the host cells is decreased or eliminated. Modified MHC molecule may be an MHC class I or class II molecule. In certain embodiments, expression of an endogenous MHC molecule may be decreased or eliminated by disrupting one or more of the MHC, β2M, TAP1, TAP2, CIITA, RFX5, RFXAP and/or RFXANK locus.

Expression of the endogenous TCR, an MHC molecule, and/or any other immunogenic molecule in the host cell can be disrupted using genome editing techniques such as Clustered regularly interspaced short palindromic repeats (CRISPR)/Cas, zinc finger nucleases (ZFNs), transcription activator-like effector nucleases (TALENs), and Meganucleases. These genome editing methods may disrupt a target gene by entirely knocking out all of its output or partially knocking down its expression. In a particular embodiment, expression of the endogenous TCR, an MHC molecule and/or any other immunogenic molecule in the host cell is disrupted using the CRISPR/Cas technique.

### Pharmaceutical Compositions

In some embodiments, the compositions comprise one or more polypeptides of the CARs and other related molecules (e.g., second CAR), polynucleotides, vectors comprising same, and cell compositions, as disclosed herein. Compositions of the present disclosure include but are not limited to pharmaceutical compositions.

In one aspect, the present disclosure provides a pharmaceutical composition comprising a polynucleotide or a recombinant vector described herein, and a pharmaceutically accepted carrier and/or excipient.

In another aspect, the present disclosure provides pharmaceutical composition comprising the CAR-modified host cells described herein and a pharmaceutically acceptable carrier and/or excipient. In some embodiments, the host cells are modified with a CAR comprising a PDZbm (i.e., PDZ CAR) disclosed herein.

In another aspect, the present disclosure provides pharmaceutical composition comprising host cells modified with a PDZ CAR and host cells modified with a PDZ CAR, and a pharmaceutically acceptable carrier and/or excipient.

Examples of pharmaceutical carriers include but are not limited to sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions.

Compositions comprising CAR-modified host cells disclosed herein may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

Compositions comprising CAR-modified host cells disclosed herein may comprise one or more of the following: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

In some embodiments, the compositions are formulated for parenteral administration, e.g., intravascular (intravenous or intraarterial), intraperitoneal, intratumoral, intraventricular, intrapleural or intramuscular administration. The parenteral preparation can be enclosed in ampoules, disposable syringes or multiple dose vials made of glass or plastic. An injectable pharmaceutical composition is preferably sterile. In some embodiments, the composition is reconstituted from a lyophilized preparation prior to administration.

In some embodiments, the CAR-modified host cells may be mixed with substances that adhere or penetrate then prior to their administration, e.g., but not limited to, nanoparticles.

### Therapeutic Methods

In one aspect, the present disclosure provides a method for treating a tumor in a subject in need thereof. A therapeutically effective amount of the CAR-modified host cells described herein or the pharmaceutical composition comprising the host cells is administered to the subject.

The term “tumor” refers to a benign or malignant abnormal growth of tissue. The term “tumor” includes cancer. Examples of tumors are, but not limited to, the soft tissue tumors (e.g., lymphomas), and tumors of the blood and blood-forming organs (e.g., leukemias), and solid tumors, which is one that grows in an anatomical site outside the bloodstream (e.g., carcinomas). Examples of cancer include, but are not limited to, carcinoma, lymphoma, blastoma, sarcoma (e.g., osteosarcoma or rhabdomyosarcoma), and leukemia or lymphoid malignancies. More particular examples of such cancers include squamous cell cancer (e.g., epithelial squamous cell cancer), adenosquamous cell carcinoma, lung cancer (e.g., including small-cell lung cancer, non-small cell lung cancer, adenocarcinoma of the lung, squamous carcinoma of the lung), cancer of the peritoneum, hepatocellular cancer, gastric or stomach cancer (e.g., including gastrointestinal cancer, pancreatic cancer), cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, primary or metastatic melanoma, multiple myeloma and B-cell lymphoma, non-Hodgkin's lymphoma, Hodgkin's lymphoma, brain (e.g., high grade glioma, diffuse pontine glioma, ependymoma, neuroblastoma, or glioblastoma), as well as head and neck cancer, and associated metastases. Additional examples of tumors can be found in The Merck Manual of Diagnosis and Therapy, 19th Edition, § on Hematology and Oncology, published by Merck Sharp & Dohme Corp., 2011 (ISBN 978-0-911910-19-3); The Merck Manual of Diagnosis and Therapy, 20th Edition, § on Hematology and Oncology, published by Merck Sharp & Dohme Corp., 2018 (ISBN 978-0-911-91042-1) (2018 digital online edition at internet website of Merck Manuals); and SEER Program Coding and Staging Manual 2016, each of which are incorporated by reference in their entirety for all purposes.

In some embodiments, host cells modified with a PDZ CAR, or pharmaceutical compositions thereof, are administered to a subject to treat a tumor expressing a tumor antigen disclosed herein. Non-limiting examples of tumors expressing a tumor antigen disclosed herein include acute lymphoblastic leukemia, acute myeloid leukemia, adult solid tumors and brain tumors, adrenal gland tumors, anal cancer, bile duct cancer, bladder cancer, blood cancers, bone cancer, bowel cancer, brain tumors, breast cancer, cancer of unknown primary, cancer spread to bone, cancer spread to brain, cancer spread to liver, cancer spread to lung, carcinoid, cervical cancer, children's cancers, colorectal cancer, chronic lymphocytic leukemia, chronic myeloid leukemia, colorectal cancer, ear cancer, endometrial cancer, eye cancer, follicular dendritic cell sarcoma, gallbladder cancer, gastric cancer, gastro esophageal junction cancers, germ cell tumors, gestational trophoblastic disease, glioma, glioblastoma, gynecological cancer, hairy cell leukemia, head and neck squamous cell carcinoma, high grade gliomas, Hodgkin lymphoma, Kaposi's sarcoma, kidney cancer, large bowel and rectal neuroendocrine tumors, laryngeal cancer, leukemia, Linitis plastica of the stomach, liver cancer, low grade gliomas, lung cancer, lung neuroendocrine tumors (NETs), lymphoma, malignant schwannoma, mediastinal germ cell tumors, melanoma, men's cancer, merkel cell skin cancer, mesothelioma, molar pregnancy, mouth and oropharyngeal cancer, myeloma, nasal and paranasal sinus cancer, nasopharyngeal cancer, neuroblastoma, neuroendocrine tumors, neuroendocrine tumors of the pancreas, non-Hodgkin lymphoma, non-Hodgkin lymphoma in children, esophageal cancer, oral squamous cell carcinoma, ovarian cancer, pancreatic cancer, pediatric solid tumors and brain tumors, penile cancer, persistent trophoblastic disease and choriocarcinoma, pheochromocytoma, prostate cancer, pseudomyxoma peritonei, rare cancers, rectal cancer, renal cancer, retinoblastoma, salivary gland cancer, secondary cancer, signet cell cancer, skin cancer, small bowel cancer, small bowel neuroendocrine tumors, soft tissue sarcoma, stomach cancer, stomach neuroendocrine tumors, testis cancer, thymus gland tumors, thyroid cancer, tongue cancer, tonsil cancer, tumors of the adrenal gland, unknown primary cancer, urothelial, uterine cancer, vaginal cancer, vulval cancer, Wilms' tumor, and womb cancer.

In some embodiments, host cells modified with a PDZ CAR pharmaceutical compositions thereof, are administered to a subject to treat a tumor expressing additional tumor antigens disclosed herein. Non-limiting examples of additional tumor expressing additional tumor antigens include breast cancer, brain tumors such as, but not limited to, glioblastoma, high grade gliomas, low grade gliomas, head and neck cancers, liver cancers, lung cancers, melanoma, ovarian cancer, pancreatic cancer, prostate cancer, renal cancer, urothelial cancer, carcinoid, cervical cancers, colorectal cancer, endometrial cancer, lymphoma, skin cancer, stomach cancer, testis cancer, thyroid cancer and urothelial cancer.

The compositions and methods described in the present disclosure may be used to treat an autoimmune disease or disorder such as, psoriasis, vasculitis, Wegener's granulomatosis, Hashimoto's thyroiditis, Graves' disease, chronic inflammatory demyelinating polyneuropathy, Guillain-Barre syndrome, Crohn's disease, ulcerative colitis, Rheumatoid arthritis (RA), multiple sclerosis (MS), Sjögren's syndrome, Systemic lupus erythematosus, sarcoidosis, Type 1 diabetes mellitus, insulin dependent diabetes mellitus (IDDM), autoimmune thyroiditis, reactive arthritis, ankylosing spondylitis, scleroderma, polymyositis, dermatomyositis, or Myasthenia gravis.

The compositions and methods described in the present disclosure may be used to treat an infectious disease. Infectious diseases are well known to those skilled in the art, and non-limiting examples include but are not limited to infections of viral etiology such as HIV, influenza, Herpes, viral hepatitis, Epstein Bar, polio, viral encephalitis, measles, chicken pox, Papilloma virus; infections of bacterial etiology such as pneumonia, tuberculosis, syphilis; or infections of parasitic etiology such as malaria, trypanosomiasis, leishmaniasis, trichomoniasis, amoebiasis.

In some embodiments, host cells modified a PDZ CAR, or pharmaceutical compositions thereof, may be administered to a subject to treat any diseases described above.

In cases where the CAR-modified host cells also express a CD20 polypeptide, the method may further include administering an anti-CD20 antibody to the subject for removal of the isolated host cells. The anti-CD20 antibody is administered in an amount effective for sufficient removal of the isolated host cells from the subject. In some embodiments, the anti-CD20 antibody is administered in an amount effective for removal of more than 50% of the isolated host cells from the subject. For example, the anti-CD20 antibody may be administered in an amount effective for removal of more than 55%, more than 60%, more than 65%, more than 70%, more than 75%, more than 80%, more than 85%, more than 90%, more than 95%, more than 98%, more than 99%, or about 100% of the isolated host cells from the subject. The anti-CD20 antibody may be administered in an amount effective for removal of about 50% to about 70%, about 60% to about 80%, about 70% to about 90%, or about 80% to about 100% of the isolated host cells from the subject.

Non-limiting examples of anti-CD20 antibodies that can be used for removal the isolated host cells include Rituximab, Ibritumomab tiuxetan, Tositumomab, Ofatumumab, Ocrelizumab, TRU-015, Veltuzumab, AME-133v, PRO131921, and Obinutuzumab. In some embodiments, the anti-CD20 antibody is Rituximab.

In some embodiments, the therapeutic method of the present disclosure includes one or more of the following steps: (a) isolating immune cells from the subject or donor; (b) modifying the immune cells ex vivo with a polynucleotide encoding a CAR and optionally an additional protein, a second CAR, or a recombinant vector comprising the same; (c) optionally, expanding and/or activating the modified immune cells before, after and/or during step (b); (d) introducing a therapeutically effective amount of the modified immune cells into the subject, and (e) in cases when the modified immune cells comprise the CD20 suicide switch, optionally, administering an anti-CD20 antibody to the subject, wherein the anti-CD20 antibody is administered in an amounts effective for removal of the modified immune cells from the subject. The immune cells may be T-cells and/or NK cells and/or macrophages.

In some embodiments, the therapeutic method of the present disclosure includes one or more of the following steps: (a) isolating NK cells, T cells, or macrophages or from a subject; (b) genetically modifying said NK cells, T cells, or macrophages ex vivo with any of the polynucleotides or the vectors described herein; (c) optionally, expanding and/or activating said NK cells, T cells, or macrophages before, after or during step (b); and (d) introducing the genetically modified NK cells, T cells, or macrophages into the subject.

In some embodiments, the subject is human.

In some embodiments, the modified host cell is an autologous cell. In some embodiments, the modified host cell is an allogeneic cell. In cases where the host cell is isolated from a donor, the method may further include a method to prevent graft vs host disease (GVHD) and the host cell rejection.

In some embodiments of any of the therapeutic methods described above, the composition is administered in a therapeutically effective amount. The dosages of the composition administered in the methods of the disclosure will vary widely, depending upon the subject's physical parameters, the frequency of administration, the manner of administration, the clearance rate, and the like. The initial dose may be larger and might be followed by smaller maintenance doses. The dose may be administered as infrequently as weekly or biweekly, or fractionated into smaller doses and administered daily, semi-weekly, etc., to maintain an effective dosage level. It is contemplated that a variety of doses will be effective to achieve in vivo persistence of modified host cells. It is also contemplated that a variety of doses will be effective to improve in vivo effector function of modified host cells.

In some embodiments, composition comprising the modified host cells manufactured by the methods described herein may be administered at a dosage of 10² to 10¹⁰ cells/kg body weight, 10⁵ to 10⁹ cells/kg body weight, 10⁵ to 10⁸ cells/kg body weight, 10⁵ to 10⁷ cells/kg body weight, 10⁷ to 10⁹ cells/kg body weight, or 10⁷ to 10⁸ cells/kg body weight, including all integer values within those ranges. The number of modified host cells will depend on the therapeutic use for which the composition is intended for.

Modified host cells may be administered multiple times at dosages listed above. The modified host cells may be allogeneic, syngeneic, xenogeneic, or autologous to the patient undergoing therapy.

The compositions and methods described in the present disclosure may be utilized in conjunction with other types of therapy for tumors, such as chemotherapy, surgery, radiation, gene therapy, and so forth.

It is also contemplated that when used to treat various diseases/disorders, the compositions and methods of the present disclosure can be utilized with other therapeutic methods/agents suitable for the same or similar diseases/disorders. Such other therapeutic methods/agents can be co-administered (simultaneously or sequentially) to generate additive or synergistic effects. Suitable therapeutically effective dosages for each agent may be lowered due to the additive action or synergy.

In some embodiments of any of the above therapeutic methods, the method further comprises administering to the subject one or more additional compounds selected from the group consisting of immuno-suppressives, biologicals, probiotics, prebiotics, and cytokines (e.g., IFN or IL-2).

As a non-limiting example, the disclosure can be combined with other therapies that block inflammation (e.g., via blockage of IL1, INFα/β, IL6, TNF, IL23, etc.).

The methods and compositions of the disclosure can be combined with other immunomodulatory treatments such as, e.g., therapeutic vaccines (including but not limited to GVAX, DC-based vaccines, etc.), checkpoint inhibitors (including but not limited to agents that block CTLA4, PD1, LAG3, TIM3, etc.) or activators (including but not limited to agents that enhance 4-1BB, OX40, etc.). The methods of the disclosure can be also combined with other treatments that possess the ability to modulate NKT function or stability, including but not limited to CD1d, CD1d-fusion proteins, CD1d dimers or larger polymers of CD1d either unloaded or loaded with antigens, CD1d-chimeric antigen receptors (CD1d-CAR), or any other of the five known CD1 isomers existing in humans (CD1a, CD1b, CD1c, CD1e). The methods of the disclosure can also be combined with other treatments such as midostaurin, enasidenib, or a combination thereof.

Therapeutic methods of the disclosure can be combined with additional immunotherapies and therapies. For example, when used for treating tumors, the compositions of the disclosure can be used in combination with conventional therapies, such as, e.g., surgery, radiotherapy, chemotherapy or combinations thereof, depending on type of the tumor, patient condition, other health issues, and a variety of factors. In certain aspects, other therapeutic agents useful for combination tumor therapy with the inhibitors of the disclosure include anti-angiogenic agents. Many anti-angiogenic agents have been identified and are known in the art, including, e.g., TNP-470, platelet factor 4, thrombospondin-1, tissue inhibitors of metalloproteases (TIMP1 and TIMP2), prolactin (16-Kd fragment), angiostatin (38-Kd fragment of plasminogen), endostatin, bFGF soluble receptor, transforming growth factor beta, interferon alpha, soluble KDR and FLT-1 receptors, placental proliferin-related protein, as well as those listed by Carmeliet and Jain (2000). In one embodiment, the modified host cells of the disclosure can be used in combination with a VEGF antagonist or a VEGF receptor antagonist such as anti-VEGF antibodies, VEGF variants, soluble VEGF receptor fragments, aptamers capable of blocking VEGF or VEGFR, neutralizing anti-VEGFR antibodies, inhibitors of VEGFR tyrosine kinases and any combinations thereof (e.g., anti-hVEGF antibody A4.6.1, bevacizumab or ranibizumab).

Non-limiting examples of chemotherapeutic compounds which can be used in combination treatments of the present disclosure include, for example, aminoglutethimide, amsacrine, anastrozole, asparaginase, azacitidine, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, decitabine, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, irinotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic compounds may be categorized by their mechanism of action into, for example, following groups: anti-metabolites/anti-tumor agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

In various embodiments of the methods described herein, the subject is a human. The subject may be a juvenile or an adult, of any age or sex.

In accordance with the present disclosure there may be numerous tools and techniques within the skill of the art, such as those commonly used in molecular biology, pharmacology, and microbiology. Such tools and techniques are described in detail in e.g., Sambrook et al. (2001) Molecular Cloning: A Laboratory Manual. 3rd ed. Cold Spring Harbor Laboratory Press: Cold Spring Harbor, New York; Ausubel et al. eds. (2005) Current Protocols in Molecular Biology. John Wiley and Sons, Inc.: Hoboken, NJ; Bonifacino et al. eds. (2005) Current Protocols in Cell Biology. John Wiley and Sons, Inc.: Hoboken, NJ; Coligan et al. eds. (2005) Current Protocols in Immunology, John Wiley and Sons, Inc.: Hoboken, NJ; Coico et al. eds. (2005) Current Protocols in Microbiology, John Wiley and Sons, Inc.: Hoboken, NJ; Coligan et al. eds. (2005) Current Protocols in Protein Science, John Wiley and Sons, Inc.: Hoboken, NJ; and Enna et al. eds. (2005) Current Protocols in Pharmacology, John Wiley and Sons, Inc.: Hoboken, NJ.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1. The PDZ Binding Moiety Scaffolding Anchor Enhances CAR NK Cell Synapse Formation

It was theorized that synapse modulation or tuning to an increasingly ordered state would result in an efficient and effective CAR. NK cells were selected for use in the present Examples since they have an innate ability to kill tumor cells and contain ˜5- to 7-fold more lytic granules compared to T cells^{12,13}. Likewise, NK cells are not known to cause graft versus host disease, and tumor cells have a reduced ability to evade attack due to the multiple targeting paradigms that are employed outside of the CAR:Antigen recognition axis¹⁴.

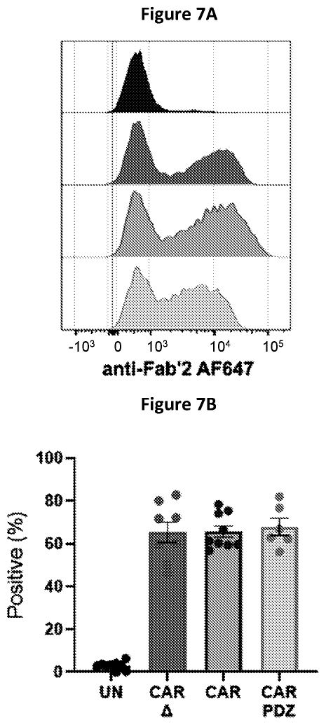

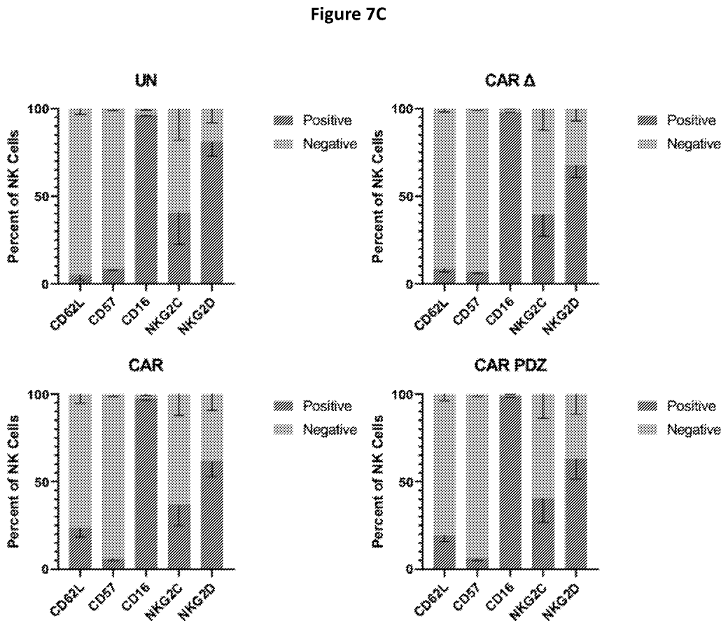

To improve CAR synapse formation, Postsynaptic density-95, Discs large, and Zona occludens 1 binding motifs (PDZbms) was selected as a focal point of the present disclosure. There are roughly 400 proteins that contain PDZ domains, and many of these are typically associated with highly polarized epithelial, endothelial, and neuronal cells¹⁵. Additionally, there are some PDZbms that aid in synapse formation and establishment of polarity of immune cells⁴. One of these motif-containing proteins, Cytotoxic and Regulatory T cell Associated Molecule (CRTAM) is a binding partner of Scribble, a PDZ domain containing intracellular scaffolding protein and mainly described in epithelial junctions⁵. CRTAM has been studied in T and NK cells^{4,16}, and CRTAM plays a role in NK cell tumor immunosurveillance¹⁷. Thus, the PDZbm of CRTAM was selected for addition to the C-terminus (CAR.PDZ) of a CD28z CAR (CAR)¹⁸, which targets the tyrosine kinase, ephrin type-A receptor 2 (EphA2) or B7-H3, a tumor associated antigen expressed in a broad range of solid tumors^{19,20} (FIGS. 1A-1B). CAR NK cells were generated from cultured primary peripheral blood NK cells by retroviral transduction and CAR, CAR.PDZ, and a non-functional control CAR (CAR Δ) were expressed equally with no significant differences on the cell surface of NK cells across numerous donors (FIGS. 7A-7B). Additionally, the CAR modifications did not alter the immunophenotype of NK cells (FIG. 7C).

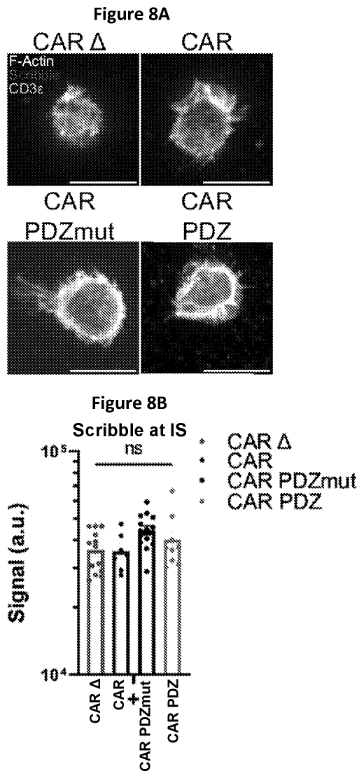

First, it was determined if the PDZbm modulates CAR NK cell synapse formation in the singular context of EphA2. Recombinant human EphA2 protein was doped to poly-L-lysine coated glass slide and CAR NK cells were allowed to incubate and interact for 0 or 30 minutes (FIG. 1I-1P). Synaptic area, downstream signaling via ZAP70 phosphorylation, and lysosomal polarization were assessed via confocal microscopy. A marked difference was found between CAR.PDZ and CAR constructs in the synaptic area (FIG. 1C) and pZAP70 accumulation at the immune synapse (FIG. 1D). The synaptic area was more condensed for CAR.PDZ and pZAP70 was increased in CAR.PDZ NK cells suggesting a more efficient signaling cascade (FIG. 1D). pERK levels were additionally assessed via flow cytometry and enhanced levels were found over 120 minutes (FIG. 17). Lysosomal polarization trended toward elevated levels in CAR.PDZ NK cells, which suggests cytotoxic vesicle recruitment to the immune synapse is enhanced (FIG. 1E). CAR.PDZ constructs were also found to induce a significantly greater accumulation of Scribble (FIG. 1F and FIG. 1G), confirming the functionality of the PDZ domain. This accumulation was time dependent as differences were not observed at 30 minutes (FIGS. 8A-8B). Furthermore, an increase in a cytoplasmic splice variant of CD3ε⁴⁴ that co-localized with Scribble was observed as determined by the Pearson correlation coefficient specifically in CAR.PDZ NK cells (FIG. 1H).

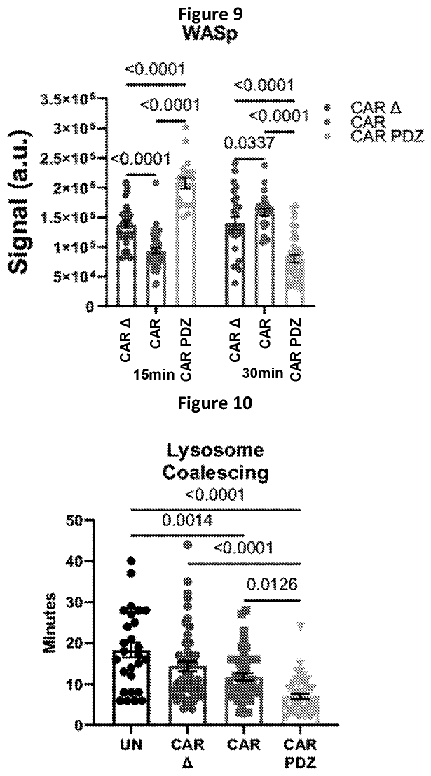

Other synaptic protein polarizations were next investigated. Given the importance of f-actin polymerization in synapse formation Wiskott/Aldrich syndrome protein (WASp) was explored. WASp regulates f-actin polymerization⁴⁵ and a rapid accumulation of WASp was found in PDZ.CAR NK cells upon antigen recognition (FIG. 9). Thus, including a PDZ domain results in improved synapse formation, as judged by a condensed synaptic area with higher levels of phosphorylation (pZAP70) and recruitment of additional signaling molecules (CD3ε).

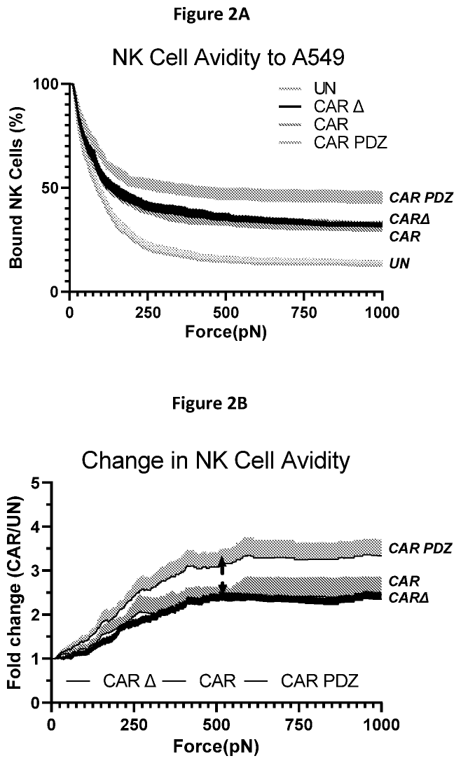

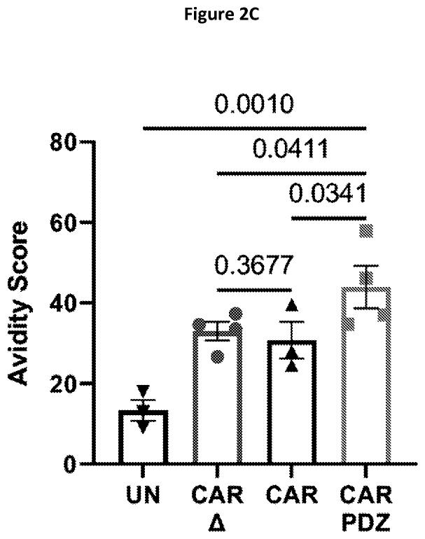

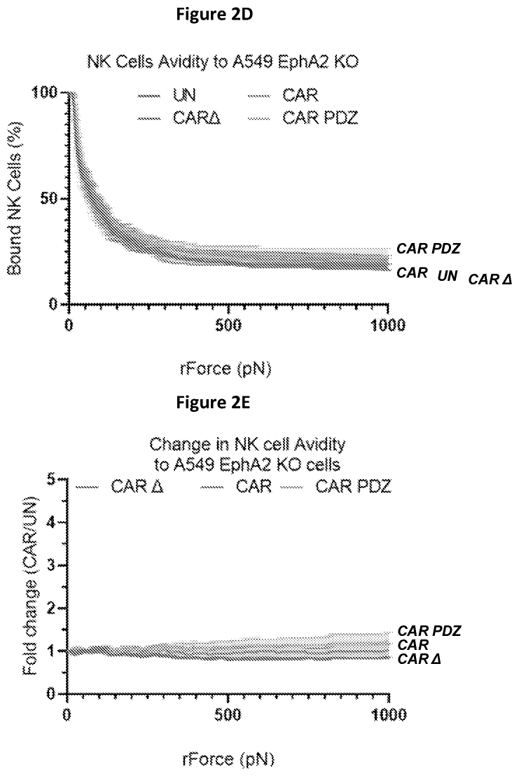

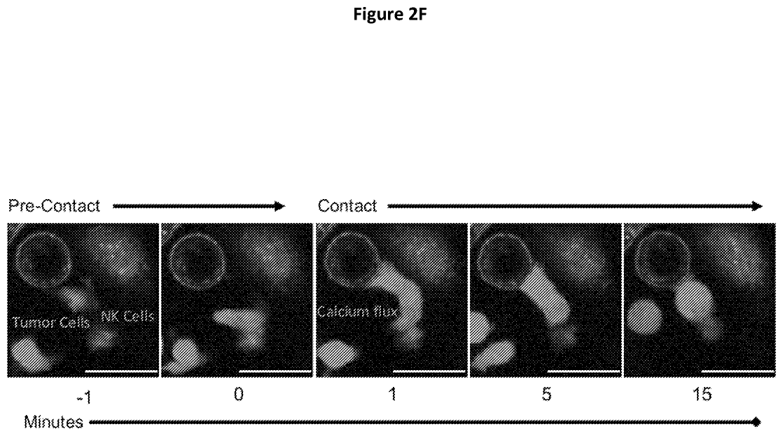

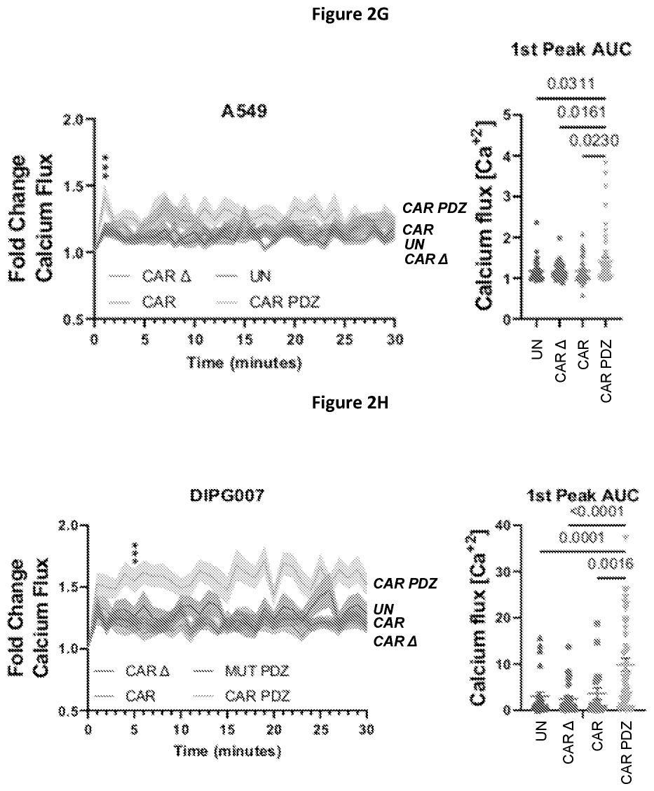

Given the multifactorial signal integration that NK cells calculate on a per cell basis^{14,21}, the complex binding between NK cells and tumor cells, specifically A549 lung adenocarcinoma cells, was explored. A single cell avidity measurement technology, z-MOVI™, that determines the precise binding force between effector and target cell was utilized. CAR.PDZ constructs were shown to have amplified binding capabilities followed by acoustic force exposure (FIG. 2A-2B). No difference between standard CAR and non-signaling CAR constructs were found, further demonstrating the lack of internal super structure being formed in a traditional CAR signaling cascade (FIG. 2C). CAR binding specificity was confirmed with a A549 cell line in which EphA2 was knocked out (KO) by CRISPR/Cas 9 gene editing (FIG. 2D and FIG. 2E).

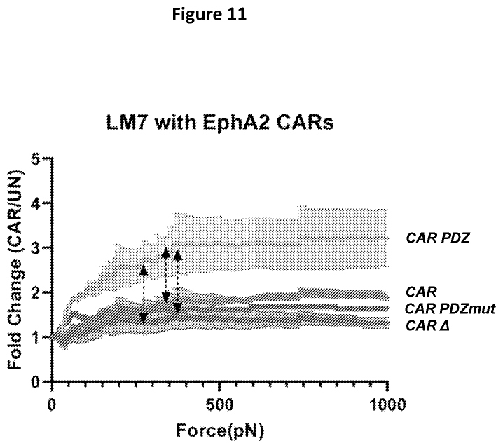

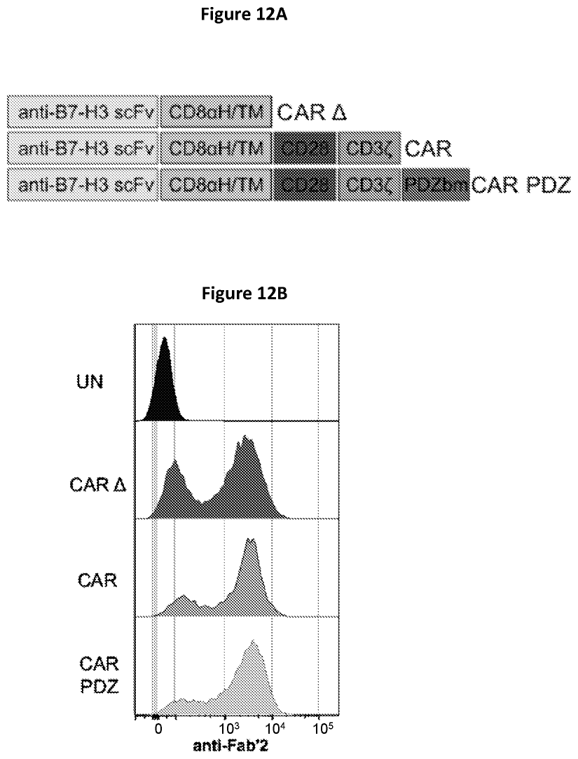

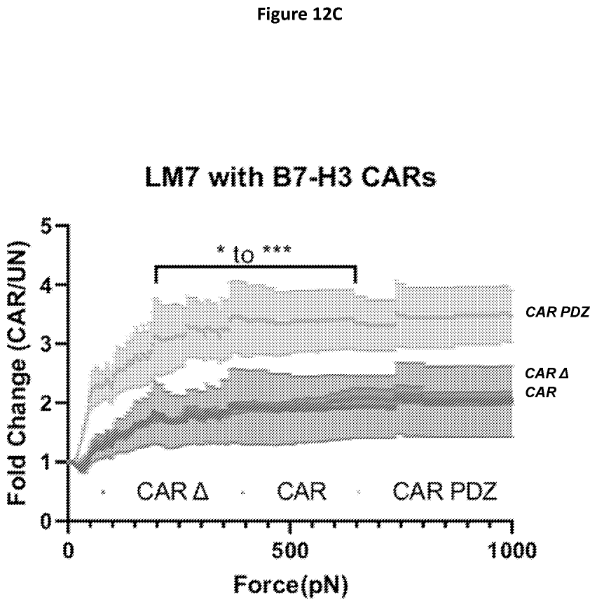

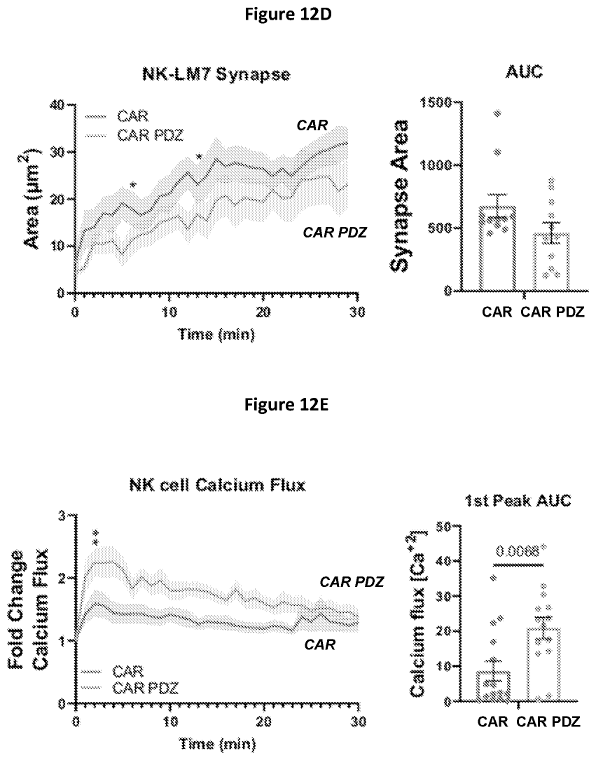

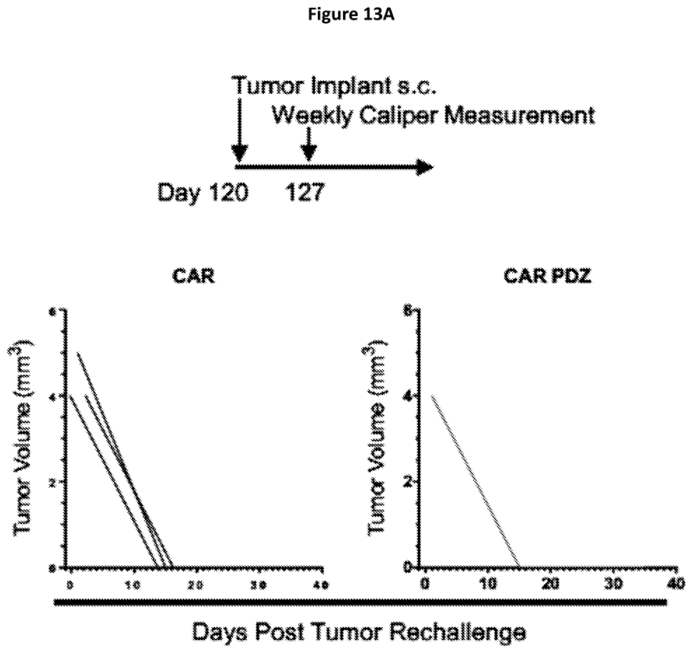

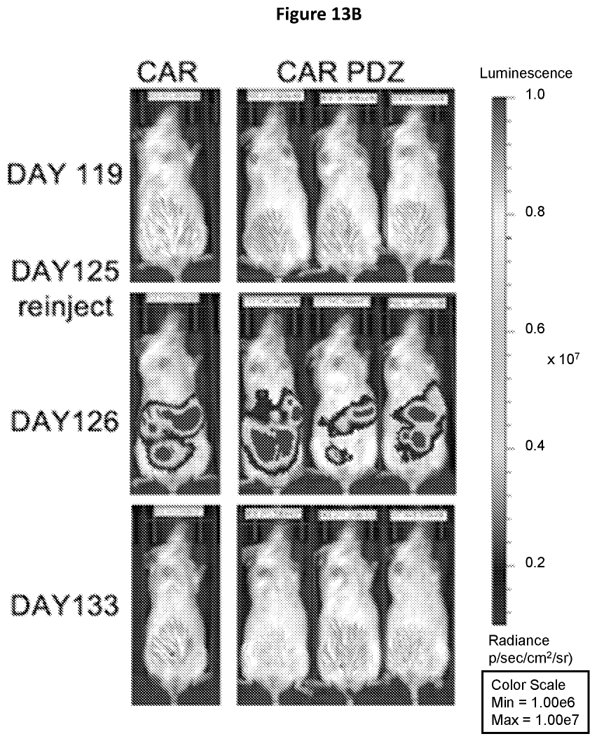

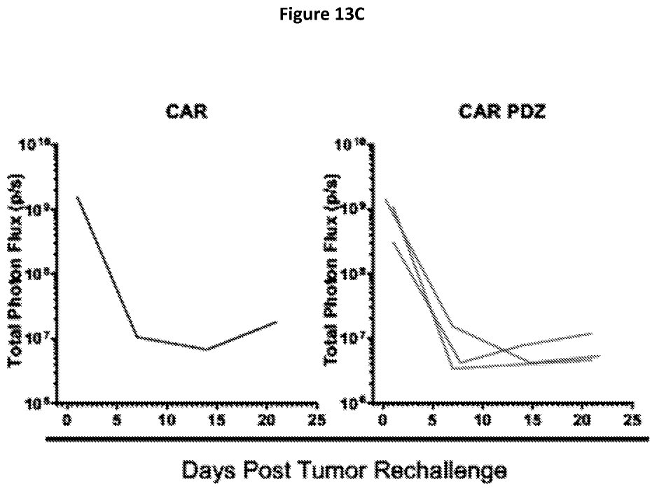

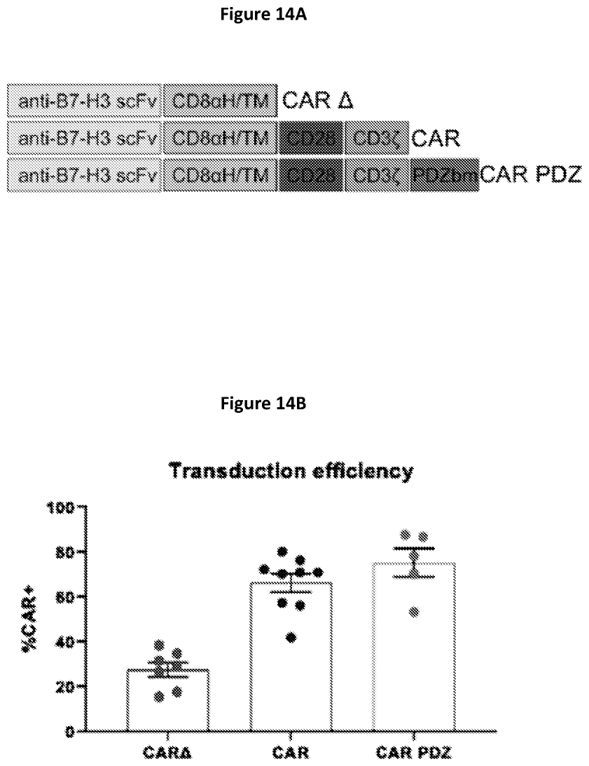

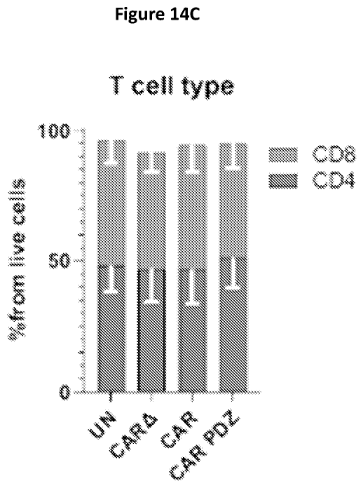

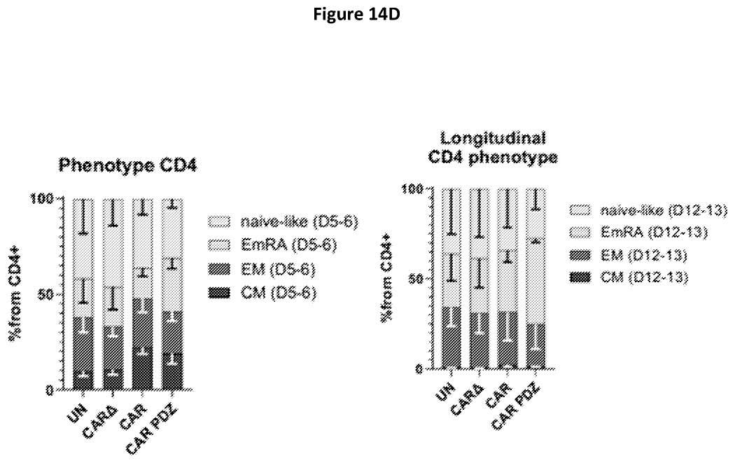

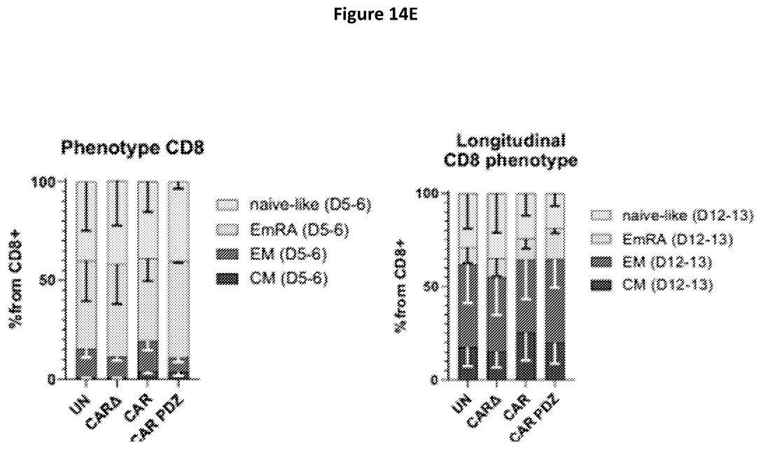

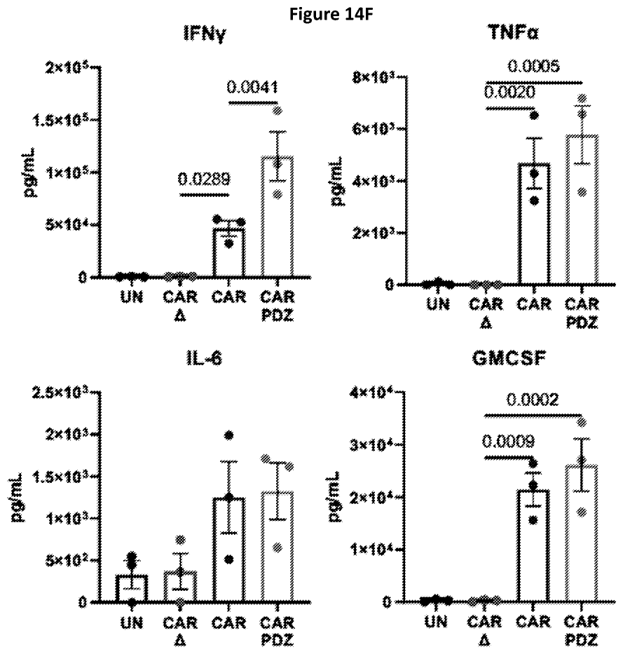

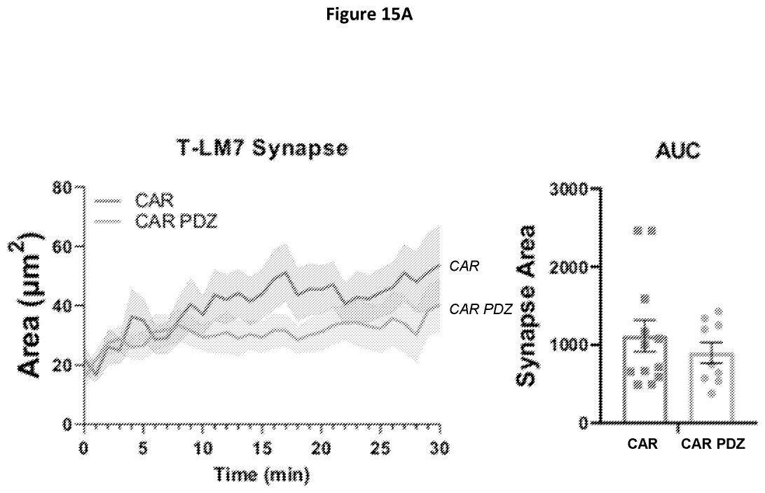

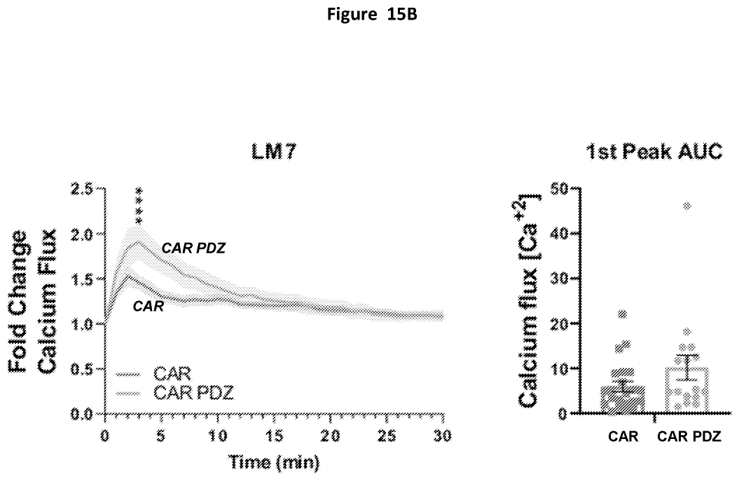

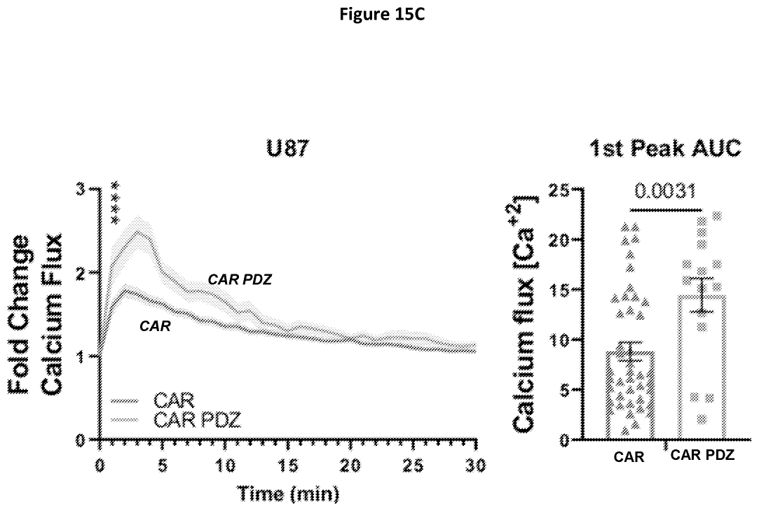

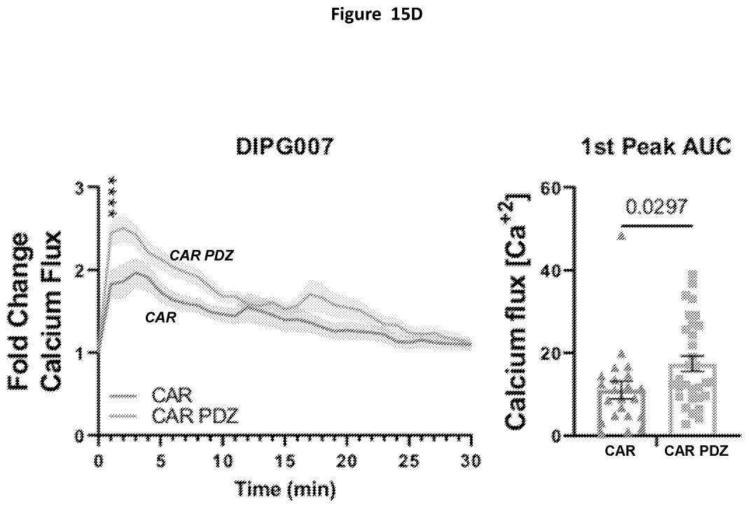

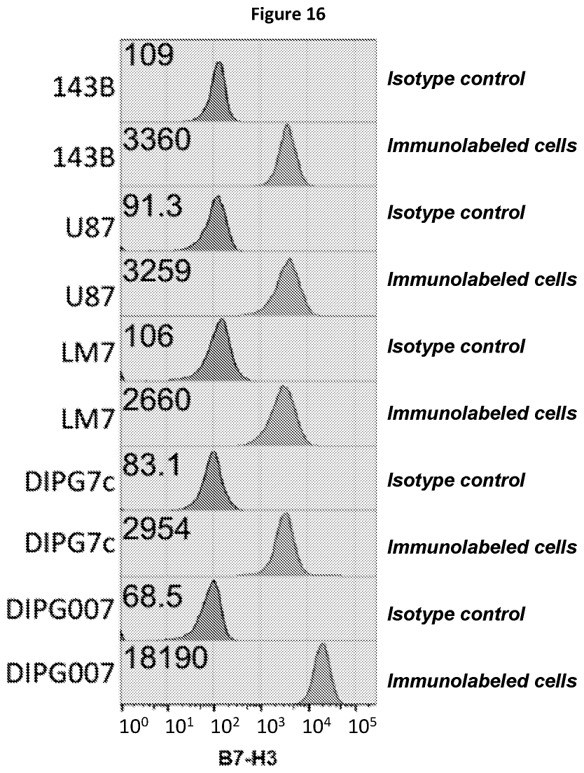

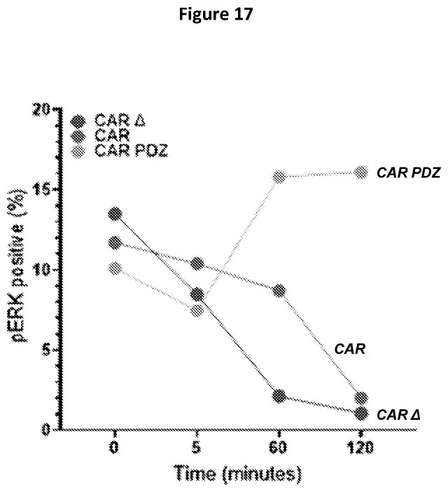

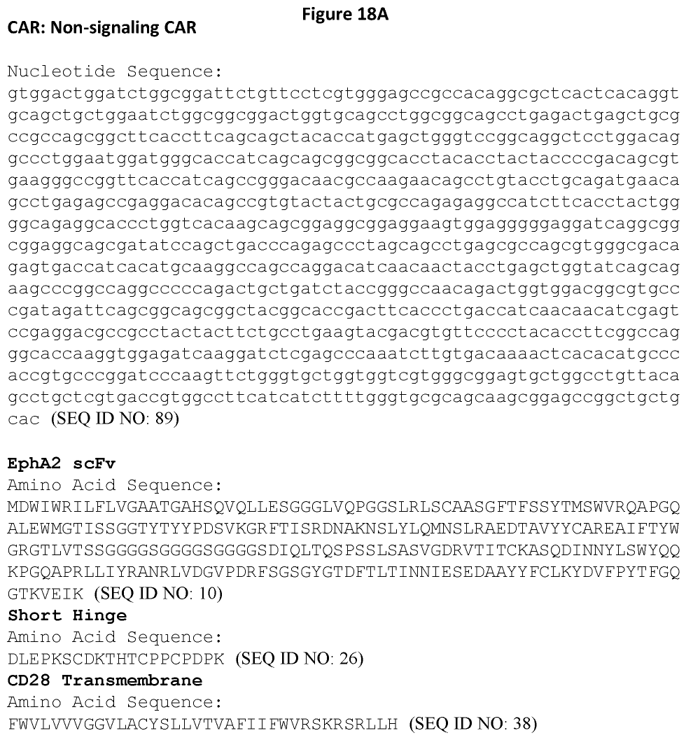

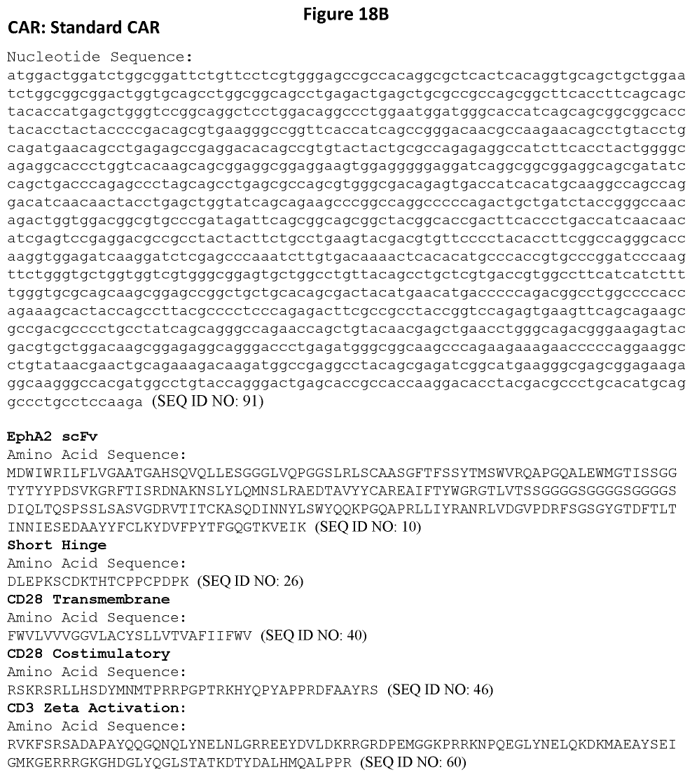

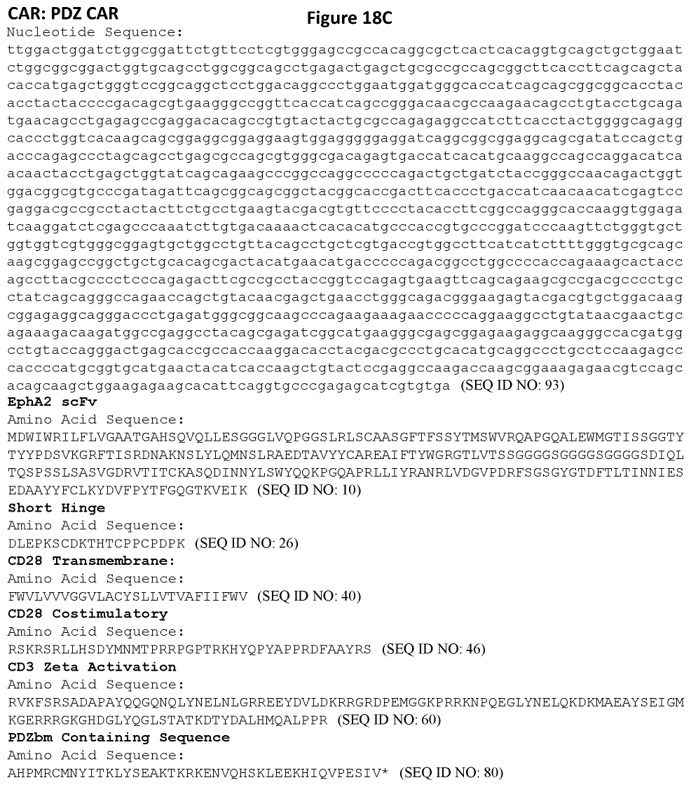

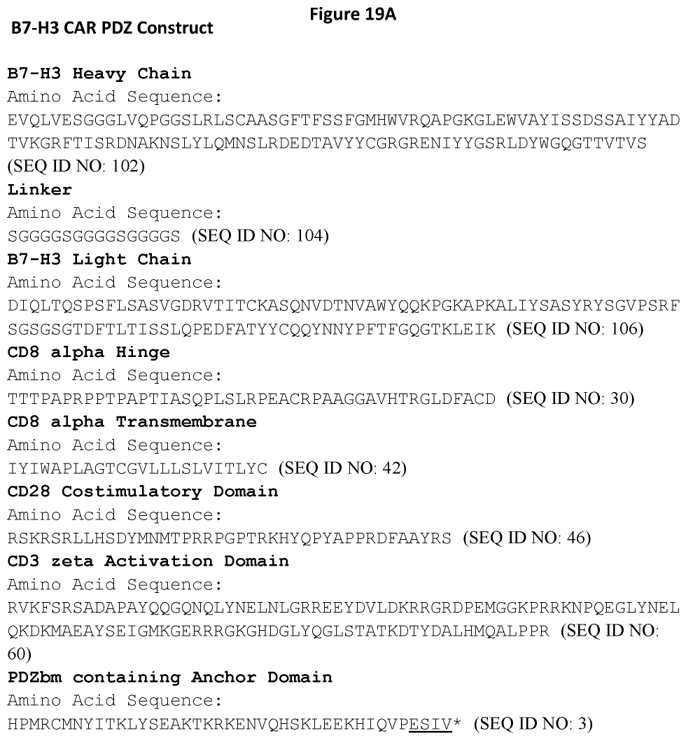

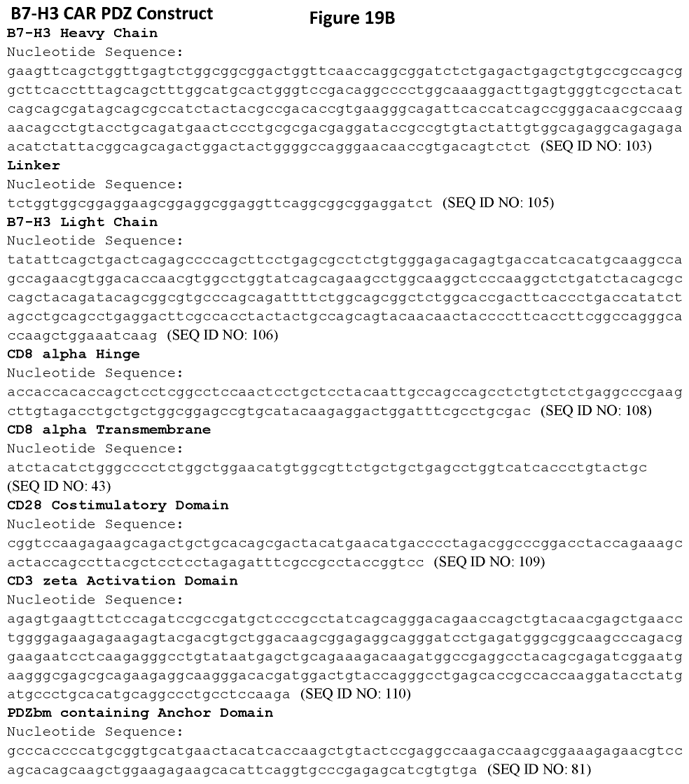

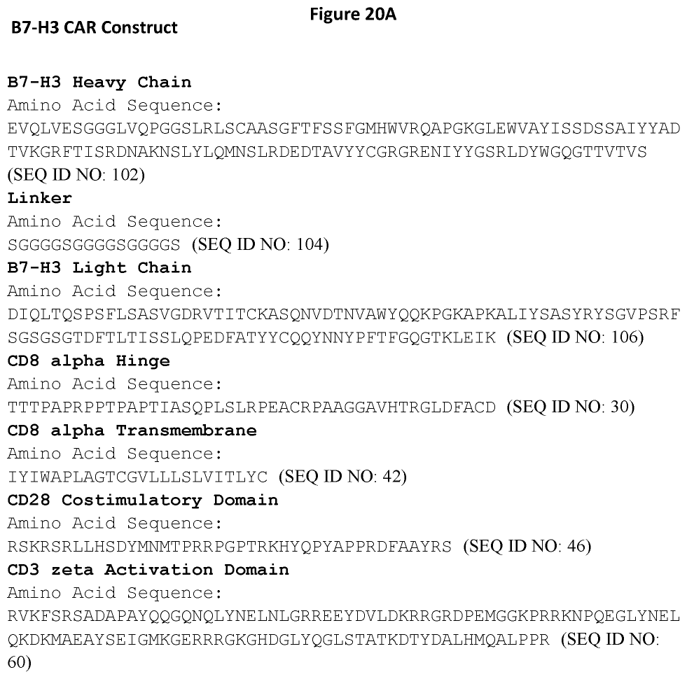

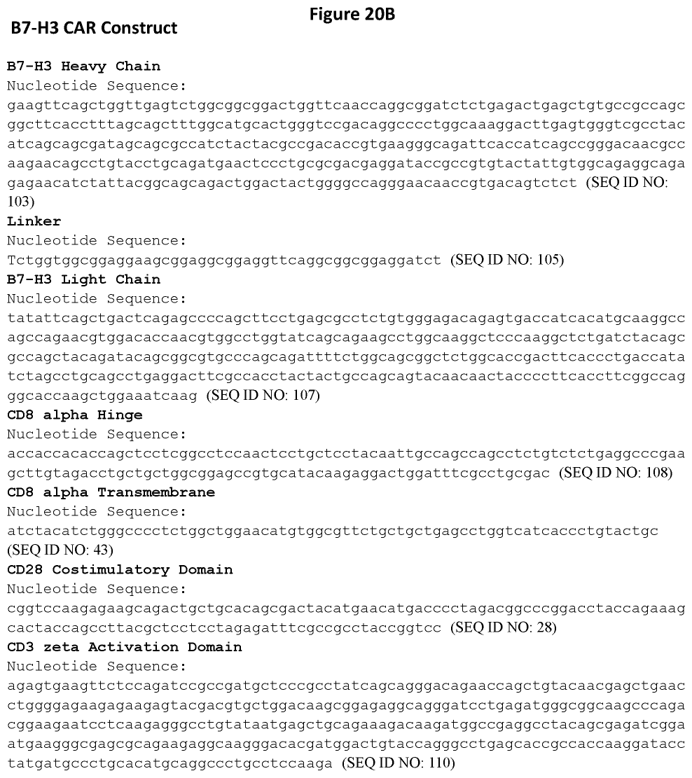

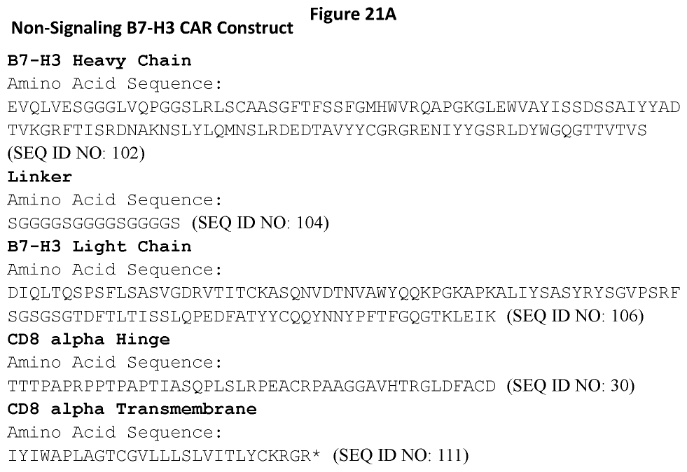

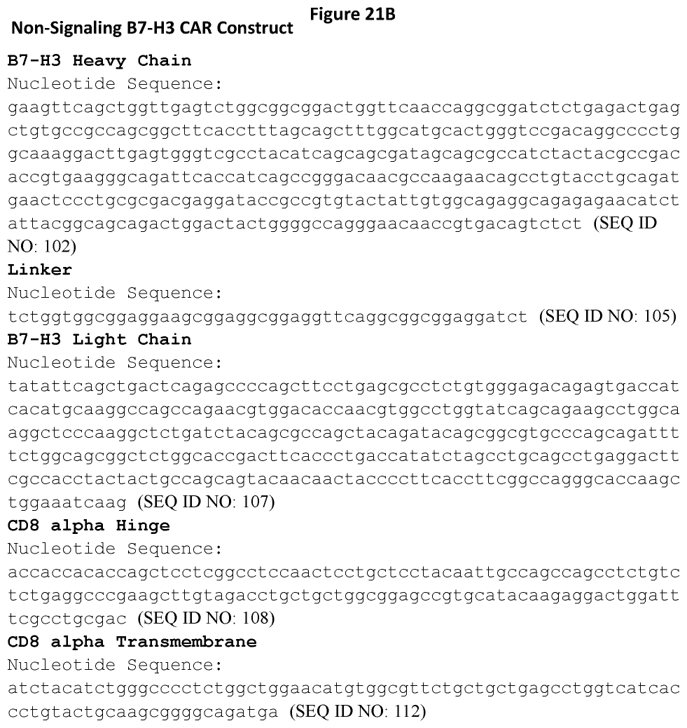

Noting the enhanced binding strength of CAR.PDZ cells over a short period of time, calcium flux, which is a first step of immune cell activation, was assessed next⁴⁶. Utilizing live cell image analysis, single cell calcium flux levels were quantified (FIG. 2F) and a greater calcium burst was found in CAR.PDZ NK cells (FIG. 2G) as well as relatively higher and sustained levels over 30 minutes in comparison to other NK cell populations (untransduced, CAR, CAR Δ). Additionally, the time from calcium flux to lysosomal aggregation was quantified in these cells. CAR.PDZ NK cells were faster at coalescing the lysosomes compared to all other groups (FIG. 10). Findings were next confirmed in the DIPG007 glioma cells, including another control: NK cells expressing CAR.PDZs in which the C-terminal amino acid of the PDZbm was mutated, to render it non-functional (CAR.PDZmut). The greatest calcium flux was observed in CAR.PDZ NK cells compared to all other groups; further, CAR Δ, CAR, and CAR.PDZmut had similar calcium flux levels (FIG. 2H). These results directly link a functional PDZbm as being important to enhance NK cell activation.

These findings were expanded upon with another cancer cell line, LM7, and similar results were found (FIG. 1I). Additionally, another CAR was created containing the PDZbm targeting B7-H3 (FIG. 12A-12B), a common tumor associated antigen studied previously^{47,48} and similar results as with the EphA2 targeting CARs were found (FIG. 12C). The synapse size created between NK cells and tumor cells was also explored. In agreement with the initial data with antigen coated slides, CAR.PDZ NK cells had a smaller synapse area upon LM7 tumor cell interaction (FIG. 12D). Consistent with the previous calcium flux experiments, CAR.PDZ NK cells display enhanced flux against LM7 cells as well (FIG. 12E). Taken together, the enhanced calcium flux, smaller synapse area, and faster lysosome congregation all indicate a more efficient synapse formation

### Example 2. CAR.PDZ NK Cells have Enhanced Cytokine Production, Cytolytic Activity, and Invasive Properties

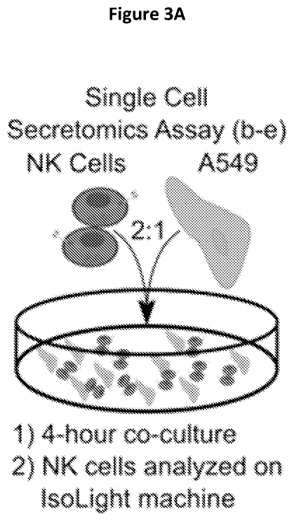

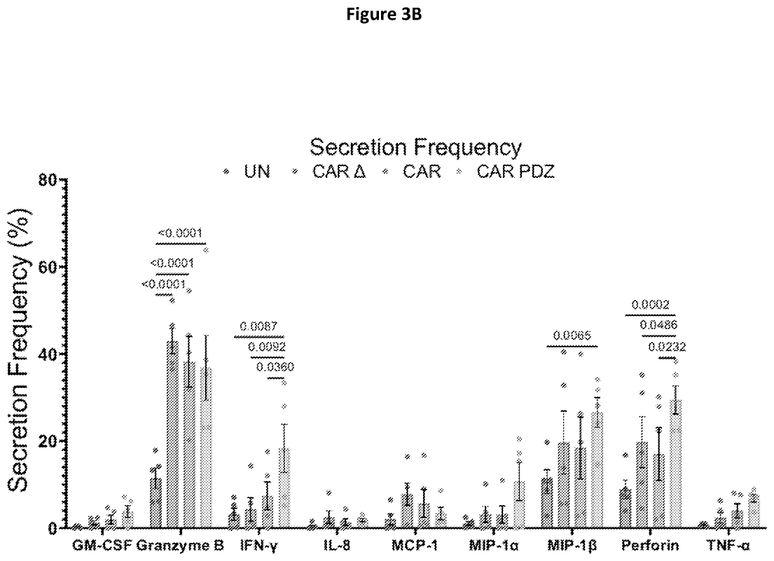

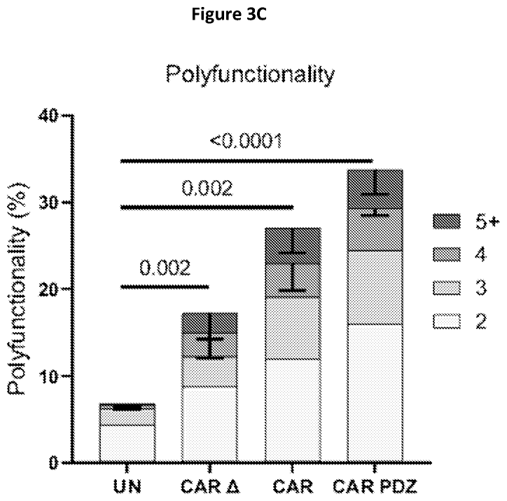

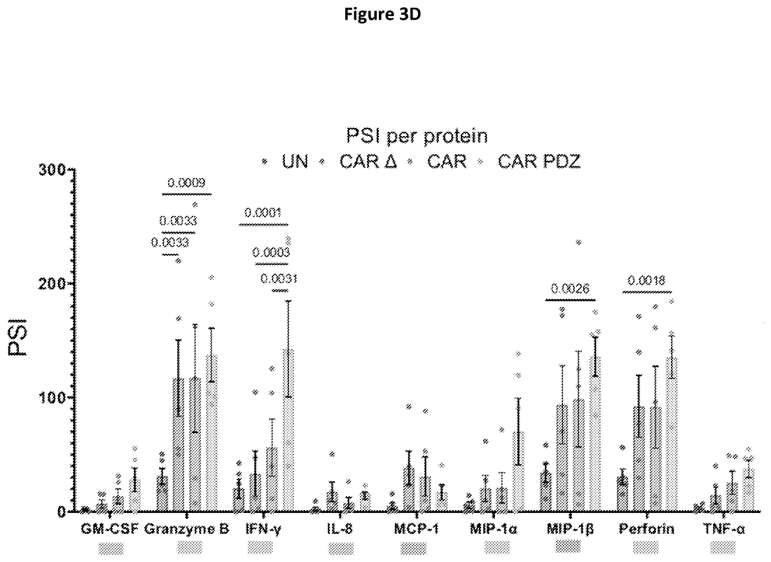

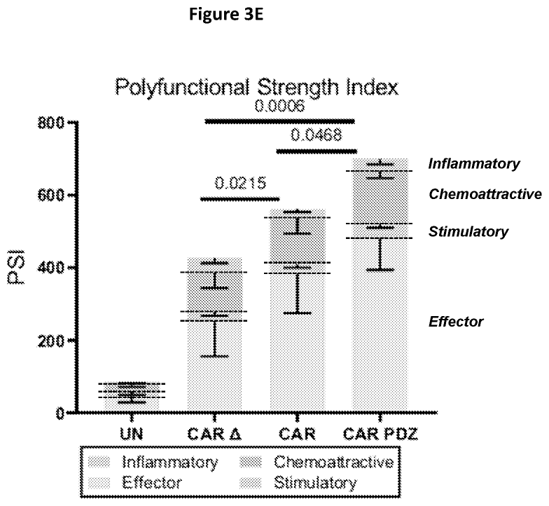

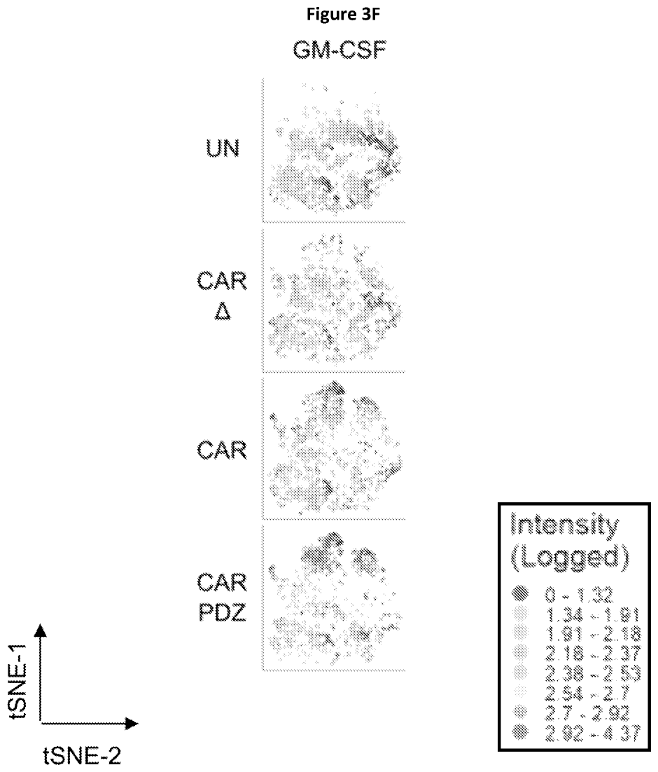

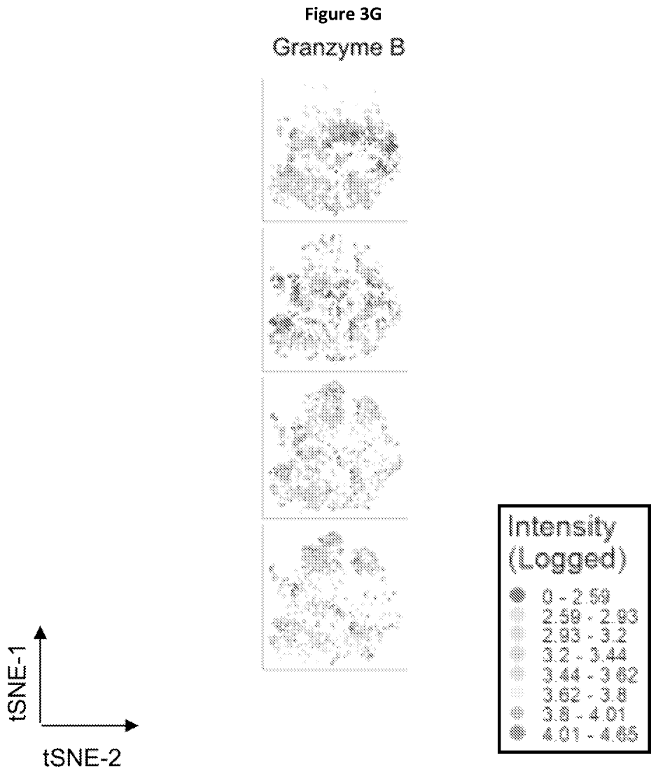

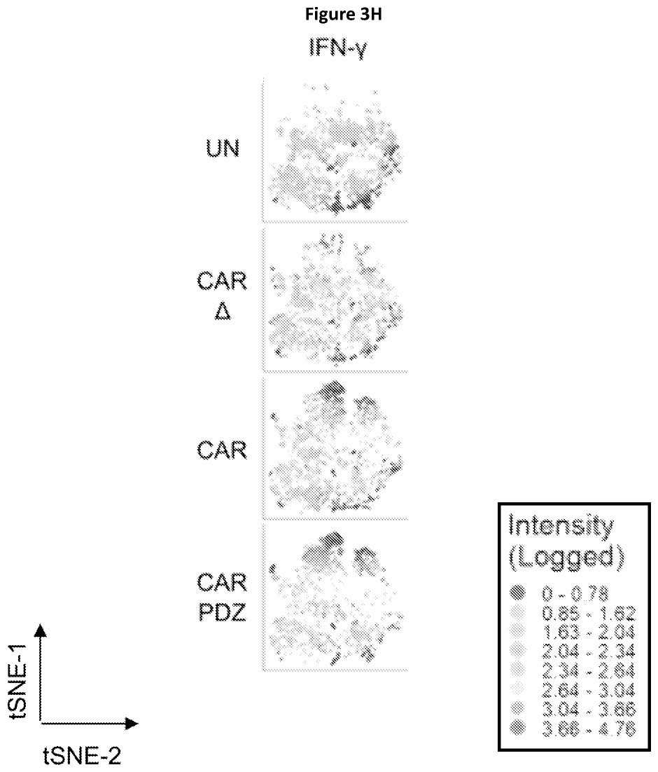

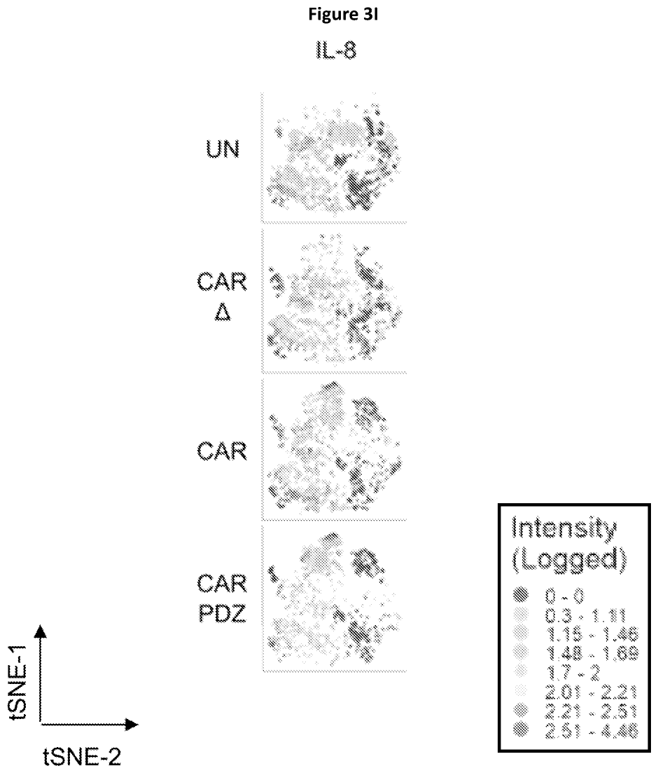

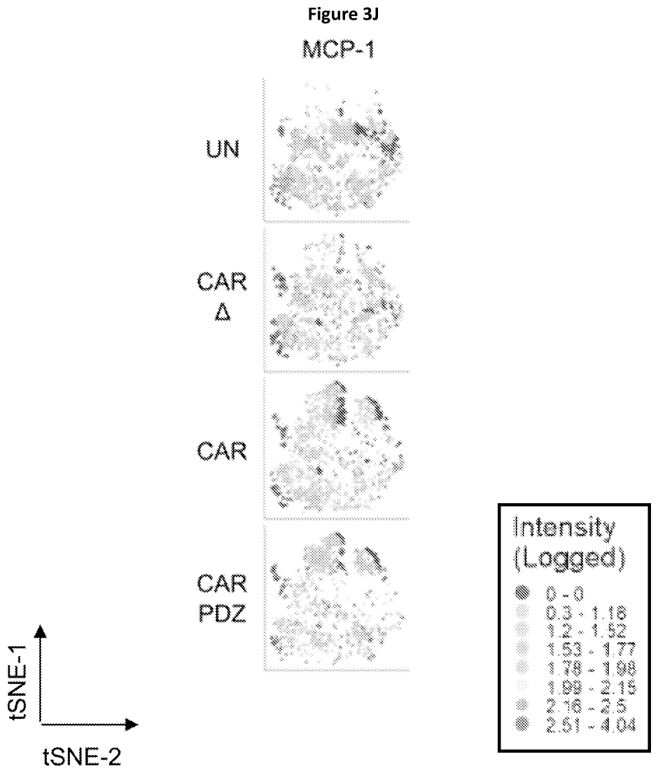

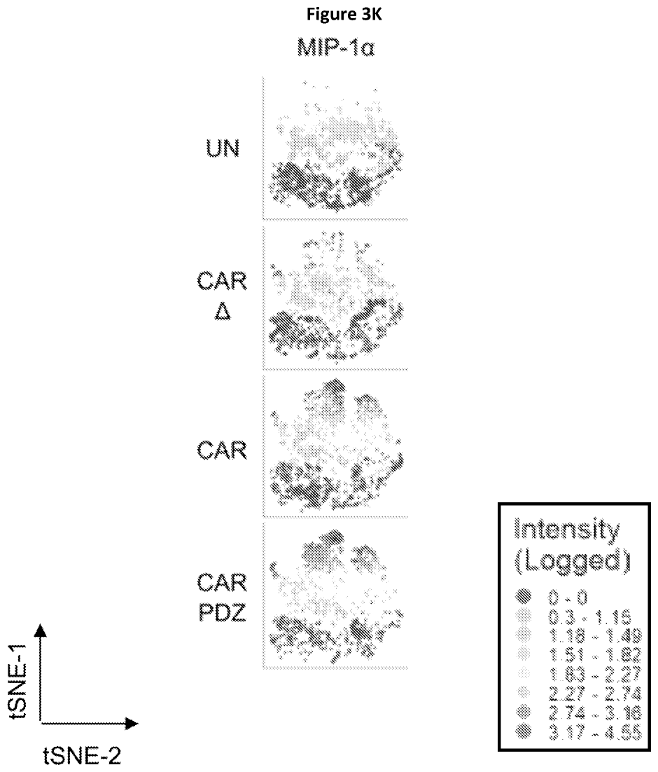

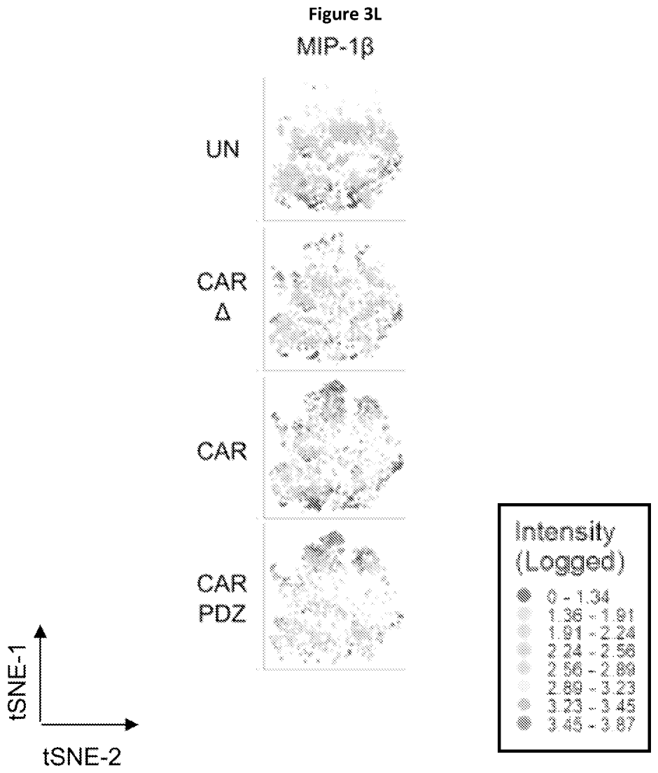

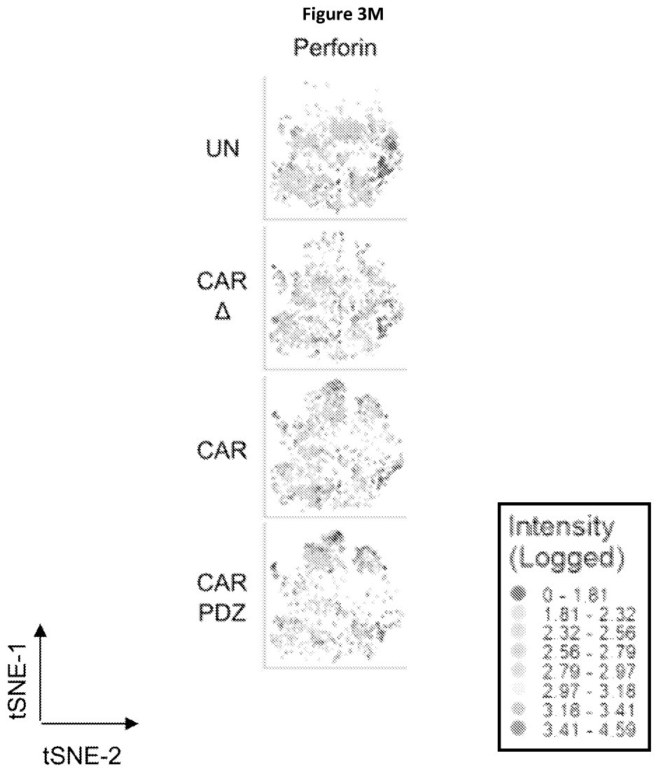

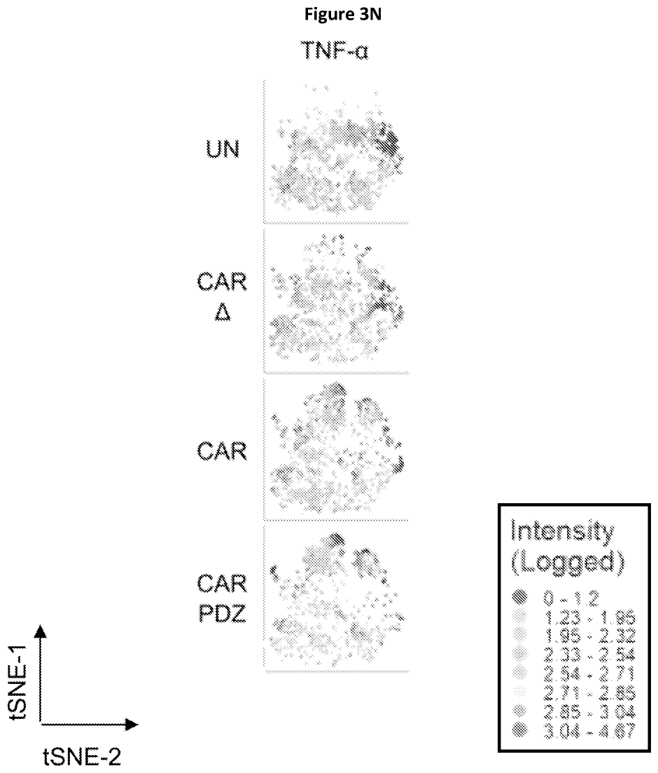

The functional consequences of the amplified signaling via zeta chain of T cell receptor associated protein kinase 70 (ZAP70) of CAR.PDZ NK cells was investigated in the present Example. To explore cytokine production upon target cell interaction, a 4-hour co-culture assay with A549 cells analyzing single cell secretomic profiles for each CAR construct was employed utilizing an IsoLight™ system (FIG. 3A). In particular, there was a significant increase in the frequency of perforin- and interferon gamma (IFNγ)-secreting CAR.PDZ NK cells in comparison to CAR NK cells (FIG. 3B). The latter finding is consistent with the observed amplified signaling via ZAP70 in CAR.PDZ NK cells, since IFNγ production typically takes the highest level of activation stimulus²². An increased frequency of granulocyte-macrophage colony-stimulating factor (GM-CSF), tumor necrosis factor alpha (TNFα), macrophage inflammatory protein 1 alpha (MIP-1α), and macrophage inflammatory protein 1 beta (MIP-1β) producing CAR.PDZ NK cells versus CAR NK cells was also observed (FIG. 3B). CAR.PDZ NK cells exhibited greater polyfunctionality, as judged by their ability to secrete multiple cytokines, in comparison to other NK cell populations (FIG. 3C). To assess the quality of cytokine production, the polyfunctional strength index (PSI), which accounts for the frequency of cells secreting cytokine and the relative intensity of cytokine produced, was calculated. IFNγ PSI was highest in CAR.PDZ NK cells (FIG. 3E), which mirrored the secretion frequency data, and that the ‘effector cytokine’ group PSI was increased in CAR.PDZ versus CAR NK cells (FIG. 3E). Finally, spectral t-SNE mapping of the signal intensity of selected cytokines (GM-CSF, IFNγ, IL-8, TNFα), chemokines (MCP-1, MIP-1α, MIP-1β) and molecules of cytolytic granules (granzyme B, perforin) revealed a distinct grouping of cells with a unique mapping of CAR.PDZ NK cells compared to other NK cell populations (FIGS. 3F-3N). Intriguingly, CAR Δ NK cells exhibited enhanced secretions compared to untransduced NK cells and in some instances similar levels to the signaling CARs. This finding may be due to the antigen recognition domain binding target cells and giving more opportunity for endogenous receptors to engage cognate ligands and become activated.

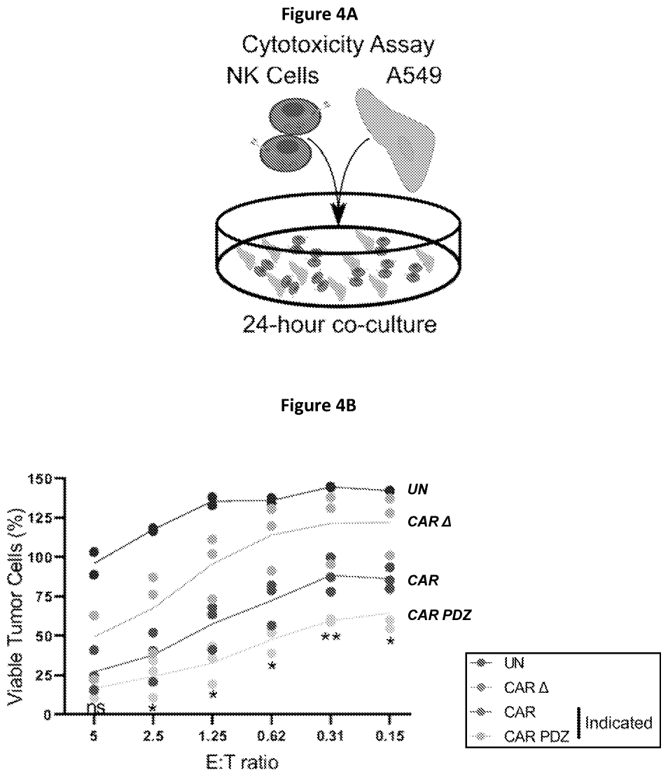

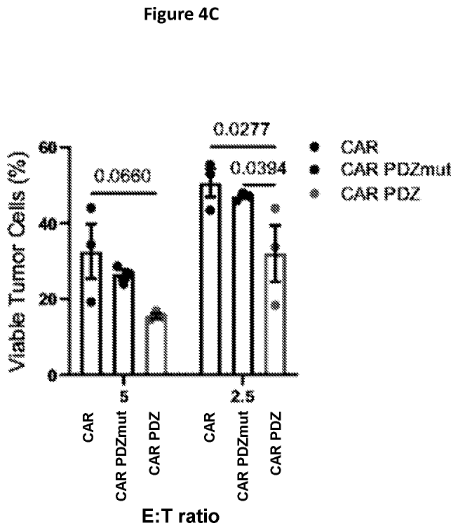

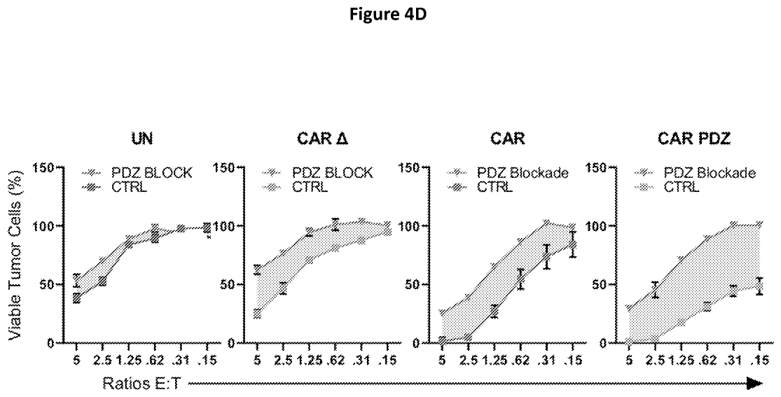

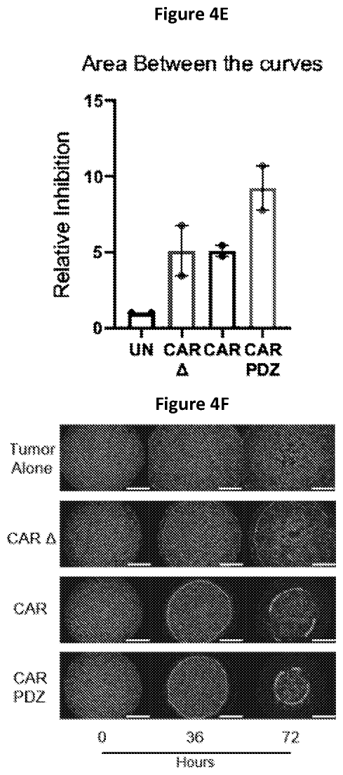

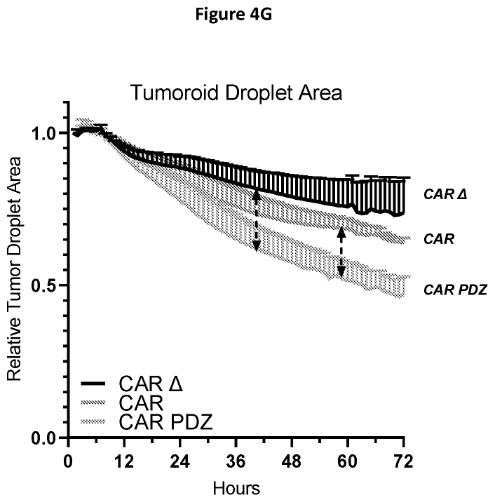

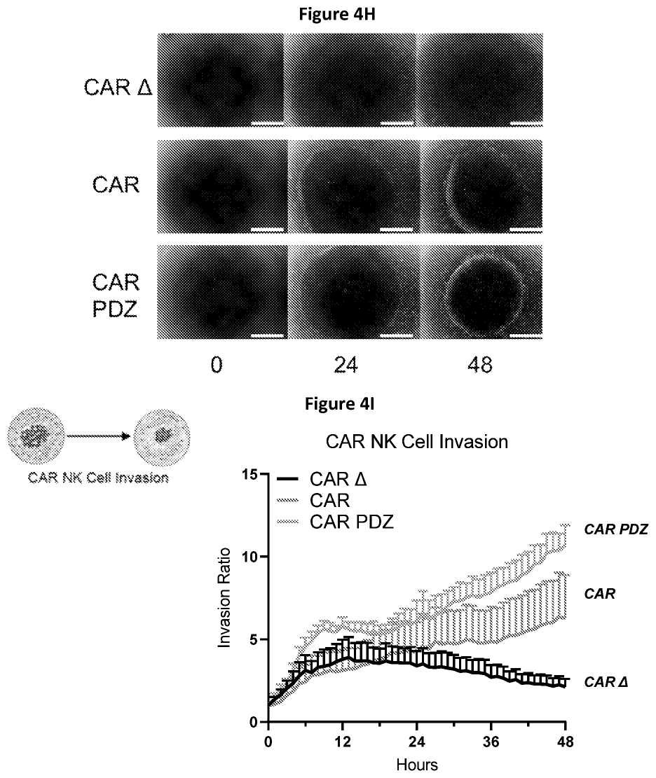

The cytolytic activity in CAR NK cells in two-dimensional (2D) and three-dimensional (3D) culture systems was next explored. In a standard 24-hour 2D MTS viability assay (FIG. 4A), CAR.PDZ NK cells exhibited superior cytolytic activity against A549 tumor cells in comparison to CAR NK cells at all evaluated effector-to-target (E:T) ratios, except for the highest E:T ratio (FIG. 4B). Assessing the mutated PDZbm CAR.PDZmut NK cells revealed a similar killing profile to standard CAR NK cells and a significantly reduced capacity compared to functional CAR.PDZ NK cells (FIG. 4C). Conversely, the functional consequences of CAR.PDZ binding to Scribble by blocking this interaction with a peptide Scribble PDZ antagonist resulted in reduced cytotoxicity (FIG. 4D). The greatest inhibitory effect was seen in CAR.PDZ NK cells as quantified by area between the curves analysis (FIGS. 4D-4E).

In a 3D assay, which consisted of a mixture of mCherry-positive 143b osteosarcoma cells and collagen in droplets surrounded by a ring of NK cells in Matrigel (FIG. 4F), CAR.PDZ NK cells reduced tumoroid size to a greater degree than did CAR NK cells (FIG. 4G). NK cells were then labeled with a green-fluorescent dye and tracked over 48 hours. CAR.PDZ cells also exhibited an enhanced ability to migrate to the droplet and invade to the center of the well in comparison to CAR NK cells (FIGS. 4H-4I). Thus, adding a PDZbm to CARs enhances not only their cytolytic potential, but also their migratory activity consistent with the known biological roles of Scribble in cell polarity and migration⁶.

### Example 3. CAR.PDZ NK Cells Extend Survival and Eradicate Solid Tumors In Vivo

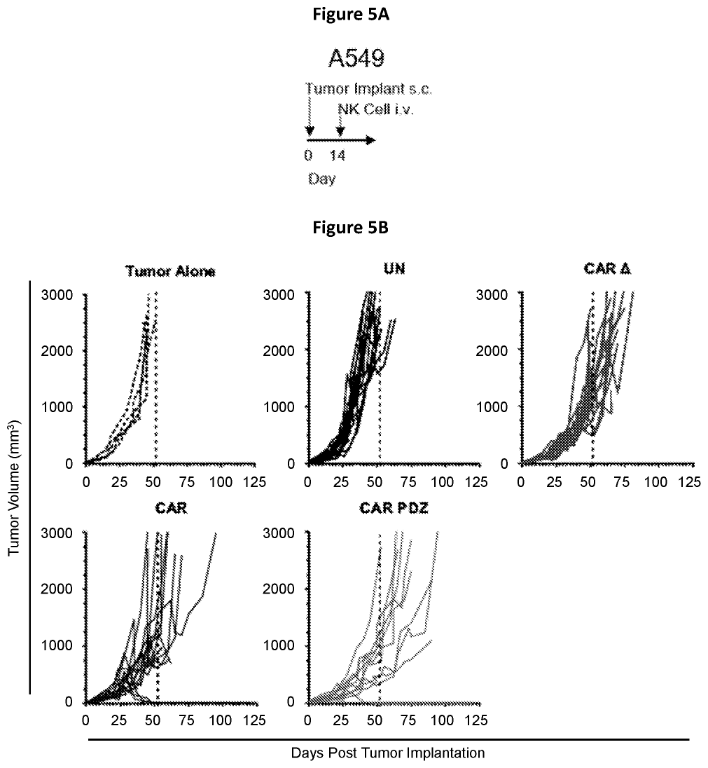

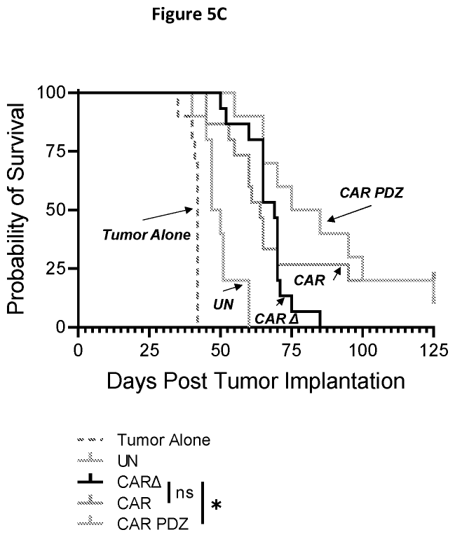

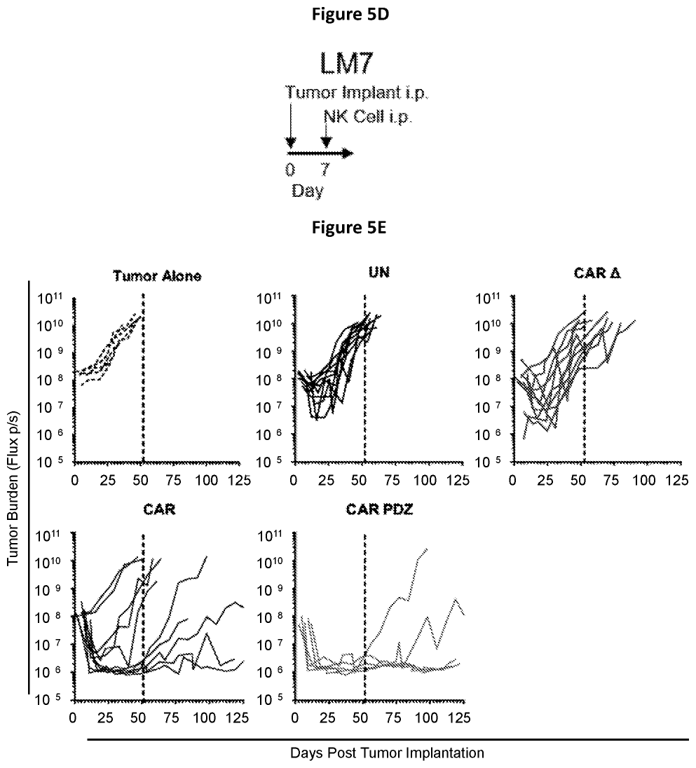

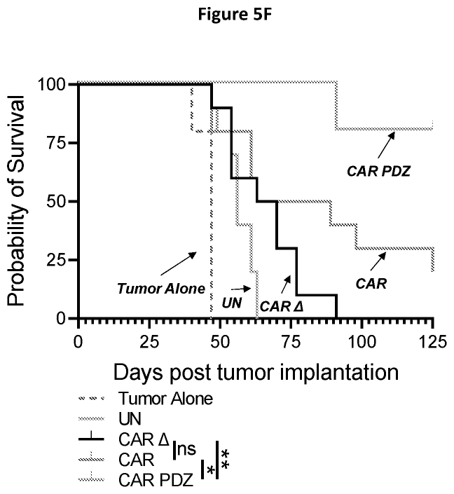

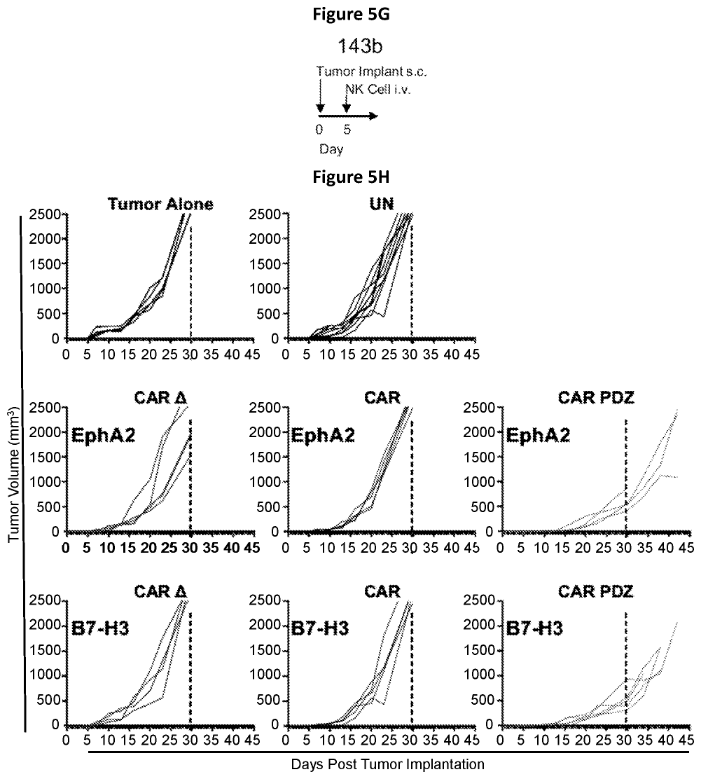

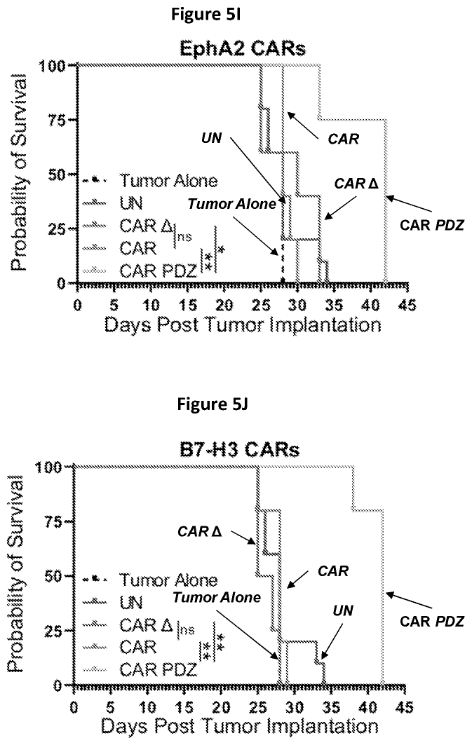

In the present Example, the anti-tumor efficacy of untransduced, CAR Δ, CAR, and CAR.PDZ NK cells were compared in three solid tumor xenograft models. Tumor cells were implanted subcutaneously in an A549 lung adenocarcinoma model and treated with NK cells intravenously 14 days later (FIG. 5A). Untreated tumors and tumors treated with untransduced NK cells showed rapid outgrowth. In contrast, CAR Δ, CAR, and CAR.PDZ NK cells had anti-tumor activity, including complete responses (CRs) for CAR and CAR.PDZ NK cells (FIG. 5B). Only mice treated with CAR.PDZ NK cells had a significant overall survival benefit in comparison to CAR Δ NK cell treated mice (FIG. 5C). The median survival of CAR.PDZ NK cell treated mice was 16 days greater than for CAR NK cell treated mice (FIG. 5C). The same CAR NK cell populations were next evaluated in a locoregional osteosarcoma model. Mice were injected intraperitoneally with firefly luciferase expressing LM7 cells followed by injection of NK cells on day 7 (FIG. 5D). Only, CAR.PDZ and CAR NK cells induced tumor regression, including CRs, as judged by bioluminescence imaging (FIG. 5E). Mice treated with CAR.PDZ NK cells had a significant survival advantage in comparison to all other treatment groups including CAR NK cells (FIG. 5F). These findings were extended to a third tumor model, 143b, which is a highly aggressive osteosarcoma. 143b cells were implanted subcutaneously and treated with NK cells 5 days later (FIG. 5G). All groups of mice except CAR.PDZ NK cell treated mice rapidly grew. This was observed in both the previous EphA2 targeting CARs and B7-H3 targeting CARs (FIG. 5H). Again, only CAR.PDZ NK treated mice had an increase in survival (FIG. 5I-5J).

The functional persistence of CAR NK cell in mice that had achieved a CR was further evaluated in both models. In the A549 model, mice were re-challenged with the original tumor cell dose ˜4 months after initial therapy. Palpable tumor nodules were measured 7 days post injection; however, large tumors never formed, and nodules had disappeared by day 14 (FIG. 13A). Likewise, LM7 tumors were rejected post rechallenge ˜4 months after initial therapy (FIGS. 13B-13C). Thus, CAR NK cells persisted and retained anti-tumor capabilities even though NSG mice do not endogenously produce cytokines that supported their survival.

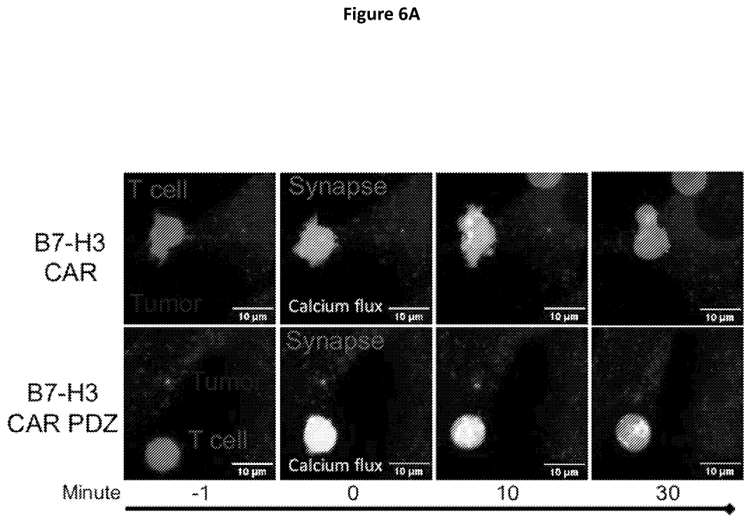

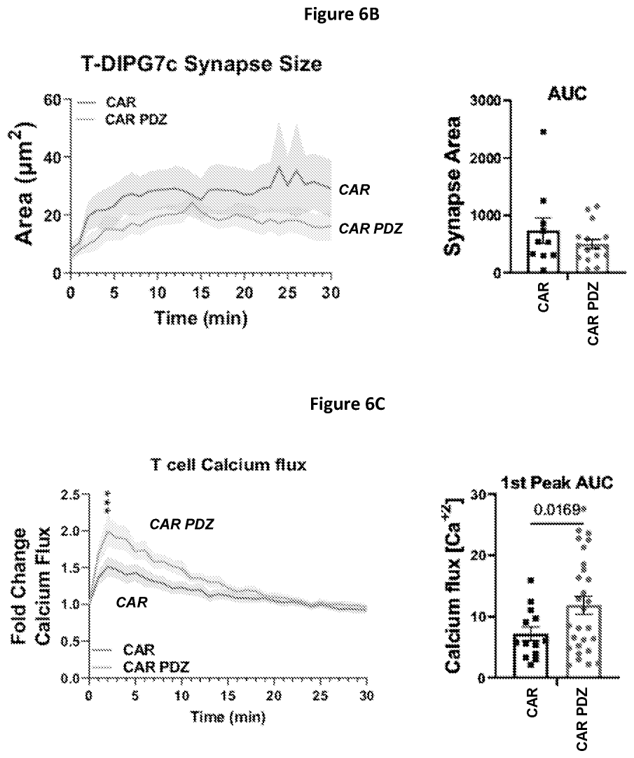

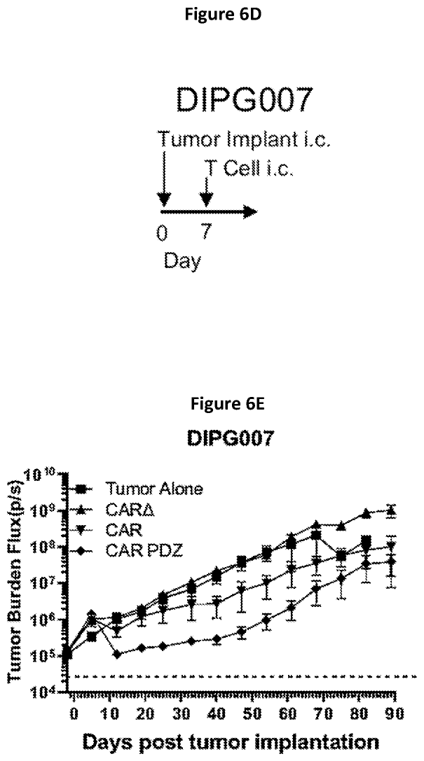

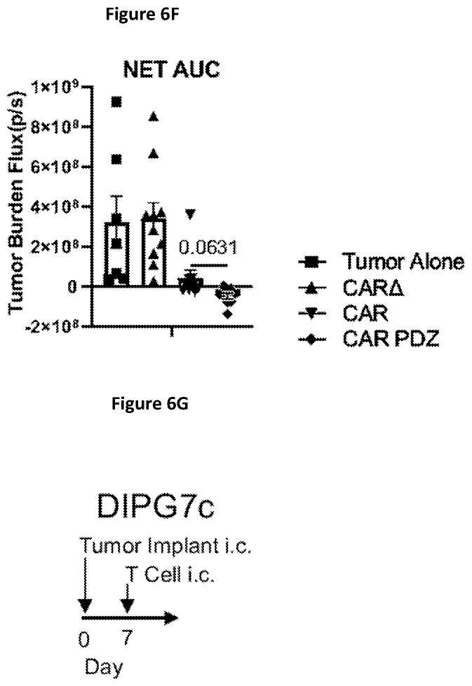

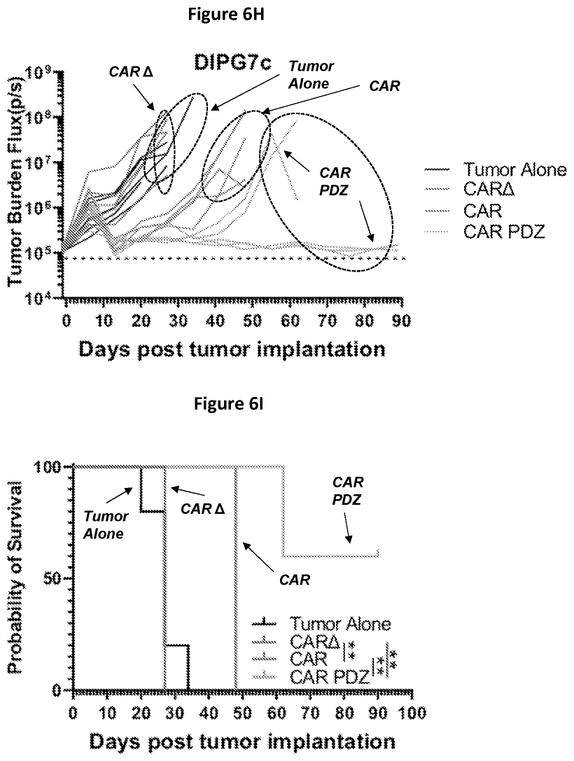

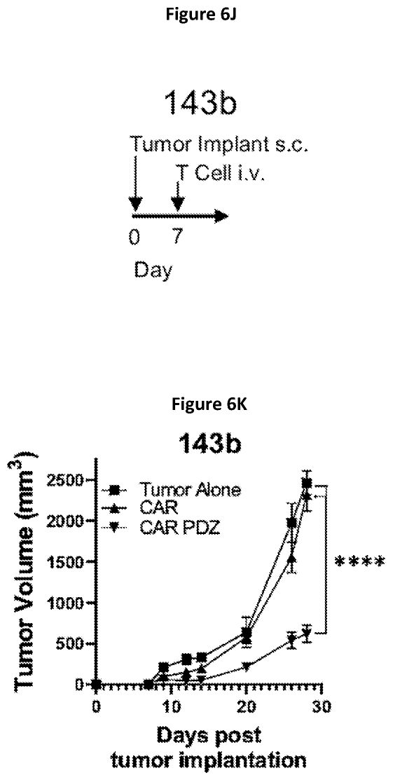

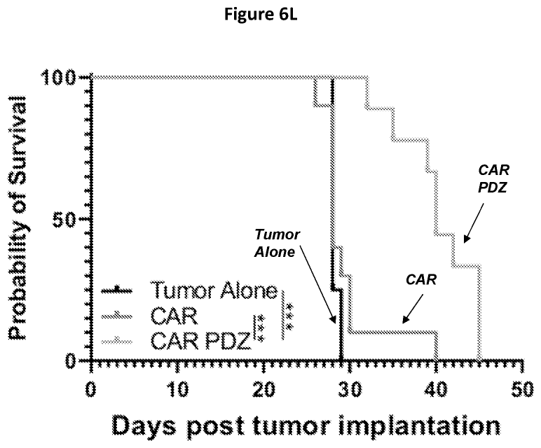

Noting the observed increases in avidity and IFNγ secretion, it was hypothesized that CAR T cells for solid tumors could be aided by the CAR design⁴⁹. To explore this, the same CAR constructs were used as before and the in vitro attributes of CAR.PDZ T cells were evaluated. No alterations in immunophenotype of CAR.PDZ T cells were found (FIG. 14A-14E). Upon co-culture with various solid tumor cells lines, CAR.PDZ T cells secreted more IFNγ and similar levels of TNFα, IL-6, and GMCSF (FIG. 14F). T cell synapse formation was next explored with DIPG7c (FIG. 6A-6B) and LM7 (FIG. 15A) tumor cells using live cell imaging along with calcium flux measurements for DIPG7c (FIG. 6C), LM7, U87, and DIPG007 (FIGS. 15B-15D). CAR.PDZ T cells had greater calcium flux and smaller synapse area as compared to CAR T cells, consistent with the notion of a more efficient synapse formation in all cases. These results were consistent with previous NK cell data indicating a cell agnostic immune synapse augmentation. Further, no functional correlation to tumor cell surface expression of the CAR antigen was found (FIG. 16).

For in vivo studies, patient derived orthotopic xenograft (PDOX) diffuse intrinsic pontine glioma (DIPG) models were a focus, as well as the highly aggressive 143b osteosarcoma model. In both DIPG models, CAR.PDZ T cells had greater antitumor activity in comparison to standard CAR T cells (FIGS. 6D-6I). Notably, full tumor eradication was observed in the DIPG7c model only in by CAR.PDZ T cells (FIGS. 6H-6I). CAR.PDZ T cells also had greater antitumor activity (FIG. 6J) in the 143b model, resulting in improved survival in comparison to mice treated with standard CAR T cells (FIG. 6K-6I). Off-target toxicities were not observed in any studies. Overall, adding a PDZbm to a CAR enhances not only the effector function of CAR NK but also CAR T cells.

The present study exploits the naturally occurring cell polarity requirements for immune cell recognition of target cells. Cell polarity is a tightly regulated process that has limited targets to augment without unintended consequences. To this end, it was found that the extant PDZbm of CRTAM would be a candidate to explore for the synapse tuned design described herein. Further, the current biological understanding of CRTAM is that it is a late phase polarity protein that aids in cell signaling after antigen recognition. By accelerating this process, a more efficient signaling CAR could be created, and effector cells imbued with enhanced functionality and longevity.

Persistence has been long thought as a hurdle of NK cell therapies, and many investigators are working on deciphering and attempting to solve this problem²³, including engineering NK cells to express IL-15, priming NK cells with cytokines to induce a “memory-like” phenotype, or culturing NK cells with various ligand expressing feeder cells²⁴ to enhance persistence. The culturing system herein involves low dose interleukin (IL)-2, 10 IU/mL, and a single, initial feeder cell stimulation. Utilizing a low stimulation level, putatively, reduces the potential for dependence on high levels of cytokine to maintain growth and survival post adoptive transfer into a harsh environment. However, directly tracking and assessing NK cell persistence needs to be performed to fully ascertain if enhanced NK cell survival occurred in vivo. Additionally, adding this domain to the C-terminal of other endogenous receptors, such as CD16, may enhance antibody-based therapeutics through antibody dependent cellular cytotoxicity.

NK cells have been derived from peripheral blood (PB)³¹, cord blood (CB)²⁵, induced pluripotent stem cell (iPSC)²⁷, or existing cell lines, e.g., NK9234. NK cells derived from these sources have been evaluated in clinical studies with an encouraging safety record to date^{24,29} The focus the study described herein was on PB-derived CAR NK cells since they are readily available from healthy donors. PB has been largely overlooked as a CAR NK cell source, especially in the context of solid tumor-redirected CAR NK cell therapy³⁰. For example, there are only a few preclinical publications that report in vivo experiments, all of which utilized repeat dosing regimens, high dose cytokine supplementation, or both³⁰. Thus, results described herein can provide impetus for the active exploration of PB-derived CAR NK cells. NK cells, regardless of source, need migratory, polarization, and internal scaffolding programs to be effectively employed as anti-cancer therapeutics.

Thus, CAR.PDZ developed herein can enhance the effector function of all NK cell products. Further studies to cross compare and modulate alternate NK-specific endodomain based CARs will be needed to fully define the role of PDZbms in NK CAR applications. Finally, since PDZbms and their interactions with scaffolding proteins is evolutionarily conserved, it is likely that PDZbms will enhance the effector function of other CAR-expressing immune cells, including T cells that are actively being explored as immunotherapeutics. The synapse-tuned CAR design described herein can enhance antigen sensitivity and, putatively, reduce the risk of cancer immune evasion by antigen loss⁵⁰, which contributes to the low overall response rate of CAR T cell therapies for solid tumors^{51,52}.

Exploration into CAR synapse tuning is at present a sparsely populated field with studies observing differences between various standard CAR designs such as bi-specific CARS^{53,54} and CARS with different costimulatory domains^{55,56}. The Examples herein reveal distinct advantages by adding a domain designed to augment the synapse. A plethora of research has been performed in optimizing CAR designs looking at external factors such as single chain variable fragment (scFv) affinity and which costimulatory domain compliments the overall activation domain. However, these efforts have been in the context of an inefficient synapse. Orienting focus to the inside of the cell to determine optimal organization of the synapse takes an orthogonal and complimentary approach to current research efforts. Re-evaluating previously discarded CAR designs with a different lens of synapse tuning may give life to previously shelved ideas. In conclusion, the present Examples demonstrate that an optimal CAR endodomain might not only have to include costimulatory and activation domains, but also a PDZbm-based anchoring domain. Thus, synapse tuning via anchor domains represents a fertile realm to explore for CAR-based immunotherapies.

### Materials and Methods Used in the Examples

Below are the methods used in the Examples described above.

### Cell Lines

143b osteosarcoma and A549 lung cancer cell lines were obtained and grown as per American Type Culture Collection (ATCC, Manassas, VA, USA) instructions. LM7, a metastatic osteosarcoma cell line, was provided by Dr. Eugenie Kleinerman (MD Anderson Cancer Center, Houston, Texas, USA) in 2011. The generation of LM7 cells expressing an enhanced green fluorescent protein firefly luciferase fusion protein (LM7.eGFP.ffLuc) was previously described³¹. K562 with modified membrane bound IL-15 and 4-1BB ligand³², feeder cells, were a generous gift from Dr. Dario Campana (National University of Singapore), and grown in Iscove Modified Dulbecco Media (IMDM) media with 10% fetal bovine serum (FBS; Hyclone Laboratories, Chicago, IL, USA). The EphA2 KO A549 cell line was generated with CRISPR/Cas9 technology using a published method³³. HSJD-DIPG007 (DIPG007) cells were cultured as described previously⁵⁷. DIPG7c cells were cultured as previously described⁵⁸. Cell lines were validated with short tandem repeat profiling performed by ATCC.

### Generation of Retroviral Vectors

The generation of the SFG retroviral vectors encoding EphA2-CARs with a CD28 costimulatory domain (CAR), a nonfunctional EphA2-CAR without a signaling domain (CAR 4) were previously described¹⁸. B7-H3 CARs were generated similarly to as previously described⁴⁷ In-Fusion cloning (Takara Bio, Kusatsu, Shiga, Japan) was used to generate the CAR.PDZ with a PDZbm attached to the C-terminus of CD3ζ domain and site directed mutagenesis Q5 (New England Biolabs, Ipswich, MA, USA) was used to do a point mutation to exchange the final Valine to an Alanine to create the CAR.PDZmut construct.

CAR.PDZ additional sequence containing PDZbm: HPMRCMNYITKLYSEAKTKRKENVQHSKLEEKHIQVPESIV* (SEQ ID NO: 3)

CAR.PDZ additional sequence containing mutated PDZbm: HPMRCMNYITKLYSEAKTKRKENVQHSKLEEKHIQVPESIA* (SEQ ID NO: 113)

The sequence of the final construct was verified by Sanger sequencing (Hartwell Center, St. Jude Children's Research Hospital). Retroviral particles were generated as previously described³⁴ by transient transfection of HEK293T cells (ATCC) with the EphA2-CAR encoding SFG retroviral vectors, Peg-Pam-e plasmid encoding MoML V gag-pol, and a plasmid encoding the RD114 envelope protein. Supernatants were collected after 48 hours, filtered, and snap-frozen for later transduction of NK cells.

### NK Cell Activation, Expansion, and Genetic Modification.

Human peripheral blood mononuclear cells (PBMCs) were obtained from whole blood of healthy donors under an Institutional Review Board (IRB) approved protocol at St. Jude Children's Research Hospital (St. Jude), after informed consent was obtained in accordance with the Declaration of Helsinki, or from de-identified elutriation chambers of leukapheresis products obtained from St. Jude donor center. Donors were less than haplo-identically matched by HLA typing. Cells were subjected to ACK Red Blood cell lysis and Ficoll Hypaque (Sigma-Aldrich, St. Louis, MO, USA) gradient separation. Cellular subtype analysis was performed with BD whole blood analysis kit on a BD Lyric flow cytometer (Becton-Dickinson, Franklin Lakes, NJ, USA). PBMCs were depleted of CD4 and CD8 using standard MACs magnetic beads (CD4: 130-045-101, CD8: 130-045-201, Miltenyi Biotec, Bergisch Gladbach, North Rhine-Westphalia, Germany). Cells were aliquoted in freezing media with 10% DMSO at 1×10⁷ cells per mL and stored in liquid nitrogen vapor phase until use. 150 Gray cesium-irradiated feeder cells were added to thawed CD4/8-depleted PBMCs at a ratio of 5-10:1 feeder to NK cells. Cells were grown in Stemcell Genix (20802-0500, Cellgenix, Portsmouth, MA, USA) growth media with 20% FBS and 10 units/mL of IL-2, (Peprotech, Rocky Hill, NJ, USA) (complete growth media). After 5-7 days cells were phenotyped and used for downstream experiments.

Genetically modified NK cells were generated as follows: Supernatants containing retroviral particles encoding CAR constructs were spun at 2000g in retronectin (T100B, Takara Bio) coated non-tissue culture 24-well plates for 90 minutes. Supernatants were removed and 250,000 NK cells were seeded per well in a volume of 1 mL of complete growth media. NK cells were incubated for 24 hours and then removed and cultured complete growth media. Modified NK cells were expanded in G-Rex 6 well plates for 10-14 days (#80240MWilson Wolf, New Brighton, MN, USA). NK cell transgene expression was assessed 3-7 days post-transduction.

### T Cell Activation, Expansion, and Genetic Modification

CAR T cells were generated via isolating PBMCs by Lymphoprep (Abbott Laboratories) gradient centrifugation and then stimulated on precoated non-tissue culture-treated 24-well plates with CD3 and CD28 antibodies (αCD3/αCD28; CD3: OKT3, CD28: 15E8; Miltenyi Biotec). The following day, rhIL-7 and rhIL-15 (IL-7:10 ng/ml; IL-15:5 ng/ml; PeproTech) were added in complete growth media RPMI (Gibco) with 10% FBS (Hyclone) and 1% Glutamax (Gibco). T cells were transduced in the same manner as NK cells detailed previously. On day 5, transduced T cells were transferred into new tissue culture 24-well plates and subsequently expanded with IL-7 and IL-15. Untransduced T cells were prepared in the same way except for the addition of retrovirus. CAR T cell expression was determined using flow cytometry on numerous days post-transduction.

### Flow Cytometry

250,000 NK cells were collected and washed twice in DPBS. Surface EphA2-CAR or B7-H3-CAR detection was determined via immunolabeling with anti-F(ab′)2-AF647 (109-606-006, Jackson Labs, Bar Harbor, ME, USA; 1:100), was utilized for detection on a BD FACS Lyric machine and analyzed with FlowJo v10 (BD Biosciences). Exemplary immunophenotyping antibodies are listed in Table 6.

TABLE 6
Exemplary immunophenotype antibodies

| Specificity | Clone | Fluorochrome | Company | Catalogue # |
|---|---|---|---|---|
| CD3 | OKT3 | Superbright 600 | Thermofisher | 63-0037-42 |
| CD56 | NCAM16.2 | PE-TX red | BD Biosciences | 564849 |
| CD62L | DREG-56 | BB515 | BD Biosciences | 464742 |
| NKG2D | ID11 | PerCPeFluor710 | Thermofisher | 46-5878-42 |
| CD57 | NK-1 | PE | BD Biosciences | 560844 |
| CD16 | REA423 | APC-H7 | BD Biosciences | 560715 |
| NKG2C | REA205 | PE Vio 770 | Miltenyi Biotec | 130-119-776 |
| CD8 | SK1 | PerCP | Biolegend | 344708 |
| CD45RA | HI100 | APC | Biolegend | 304112 |
| CD197(CCR7) | G043H7 | FITC | Biolegend | 353216 |
| CD4 | SK3 | PE/Cy7 | Biolegend | 344612 |
| CD276 | 7-517 | BV421 | BD Biosciences | 565829 |

### Single Cell Secretomics Assay

Briefly, 100,000 NK cells were labeled with cell trace violet 1:500 (ThermoFisher Scientific, Waltham, MA, USA), and co-cultured at a 2:1 ratio with A549 targets for 4-hours in a 48-well plate. NK cells were removed and washed twice with PBS and resuspended in complete growth media without IL-2. These cells were loaded onto a IsoCode single cell secretomic chip and run on the IsoLight machine that detects 32 distinct proteins³⁵. Results were analyzed on IsoSpeak version 2.8.1.0 (IsoPlexis, Branford, CT, USA).

### Cytokine Analysis

CAR T cells were incubated with various cancer cell lines for 24-hours at a 2:1 ratio. The supernatant from these co-cultures were then assessed using MILLIPLEX (MilliporeSigma, CAT: HCYTA-60K) and run on a FLEXMAP 3D (Luminex, Austin Texas, USA).

### Cytotoxicity Assay

NK cells were cytokine starved for 24-hours prior to co-culture with target cells to reduce non-specific killing of target cells. 3,000 target (A549) cells were cocultured with effectors at indicated effector-to-target (E:T) ratios for 24-hours in a 96 well plate. Cytotoxicity was quantified by a chromogenic MTS assay measured on a plate reader (Tecan, Männedorf, Switzerland) detecting remaining viable adherent tumor cells.

### Peptide Blockade

Peptides were synthesized by the Macromolecular Synthesis Core (Harwell Center, St. Jude) purity was 97% and 95% via HPLC, respectively: Negative Control Sequence: NH₂-RQIKIWFQNRRMKWKKRSWFEAWA-COOH (SEQ ID NO: 98); Scribble PDZ Blocking Sequence: NH₂-RQIKIWFQNRRMKWKKRSWFETWV-COOH (SEQ ID NO: 99). Underlined portions of sequences are from the Antennapedia protein which has been shown to allow peptide shuttling into cells and effectively block CRTAM binding³⁶. NK cells were treated with 10 micromolar of peptides for 24-hours prior to co-culture.

### Confocal Microscopy

Ag-coated coverslip preparation and NK activation: Antigen coated coverslips were prepared using NI coverslips (Fischer Scientific: #12-545-80P), which were coated with 0.5 μg/mL of rhEphA2 (R&D Systems, Minneapolis, MN, USA: #3035-A2-100) or poly-L-lysine (Sigma: #P4707) overnight at 4° C. Then, they were washed with PBS and filled with media until NK cell seeding. 200,000 NK cells were plated onto the precoated coverslips at different time points in a cell culture incubator (37° C./5% CO₂). After activation, NK cells were washed with cold PBS and fixed with 4% paraformaldehyde (PFA, Electron microscopy sciences #15710) for 10 minutes at room temperature. Fixed cells were washed twice with PBS and the remaining PFA was inactivated with blocking buffer (PBS-2% BSA (Sigma: #A9418), 1.5M Glycine (Sigma: #G8898)) for 10 min at room temperature. Cells were permeabilized by adding permeabilization buffer (PBS, 0.2% BSA, 0.05% Saponin; Sigma: #47036) for 20 minutes at room temperature. Cells were washed twice with permeabilization buffer prior primary antibody incubation diluted in permeabilization buffer, following manufacturer instructions. All the primary antibodies were incubated at 4° C. overnight. Cells were washed with permeabilization buffer and incubated with secondary antibodies for 2-hours at room temperature. Finally, cells were washed with permeabilization buffer and PBS before to let them dry for 1-hour at room temperature. Then, coverslips were mounted onto slides using fluoromount (Thermofisher Scientific: #00-4958-02).

Antibodies and probes: Primary antibodies and probes with their dilutions: Anti-human Lamp1 (1:50) (Abcam: #ab25630), Anti-Human pZAP70 (1:50) (Cell Signaling Technology: #2701L), (Abcam: Ab6160), Phalloidin-AlexaFluor647 (1:200) (Thermofisher Scientific: #A22287), Anti-Human Cde-AlexaGluro647 (1:100) (Biolegend #300422), Anti-Human Scribble (1:100) (Cell Signaling Technology, Danvers, MA, USA #4475), Anti-WASp (1:100) (AbCam: #ab75830). Secondary antibodies with their dilutions: Anti-Rabbit AlexaFluor 488 (1:200) (Thermofisher Scientific #A32731), Anti-Mouse AlexaFluor 568 (1:200) (Thermofisher Scientific #A-11004), pERK-AF488 (1:50) (Biolegend, #675508).

Image acquisition and analysis: Images were acquired in a spinning disc confocal microscope (Zeiss Axio Observer with CSU-X spinning disc), and the processing and analysis was performed with FIJI (ImageJ) software³⁷. Single cell images shown in the figures were cropped from larger field. Image brightness and contrast was manually adjusted. To analyze lysosome and pZAP70 distribution in the immune synapse, cell borders were automatically delimited by using WEKA³⁸. To segmentate every single cell using F-actin signal as a template (CellTemp), an ellipse was automatically determined (CenterTemp) at the center of the CellTemp, which had a third of the CellTemp area. The recruitment at the center of the IS was calculated by dividing the fluorescence normalized by its area from CenterTemp and CellTemp, subtracting 1. Therefore, positive values mean that the fluorescence is enriched at the center, whereas negative values mean peripheral enrichment. NK cells were normalized to the unstimulated conditions to achieve pZAP70 and Lamp1 scores.

### Live Cell Calcium Imaging

150,000 tumor cells were seeded onto μ-slide 8 well chambers (ibidi, Gräfelfing, Germany) (ibidi #80807) and incubated overnight at 37° C. and 5% CO₂. Tumor cells were labeled with CellBrite® Green membrane dye (Biotium Inc, Fremont, CA, USA) (1:2000) (biotum #30021) for 30 min and then washed and maintained in RPMI until image acquisition. 2×10⁶ CAR NK or T cells were resuspended in 1 mL of PBS and labeled with CAL520 (1:500) (ATTbioquest #21130) and celltrace violet (1:1000) (Thermofisher #C34557) for 1 hour and then washed and maintained in RPMI until image acquisition.

3×10⁵ CAR immune cells were added to each well preloaded with tumor cells, and the image acquisition was initiated upon immune cell detection in the visual field. Images were acquired in a spinning disc confocal microscope (Zeiss Axio Observer with CSU-X spinning disc), using a 63× objective. The acquisition parameters were a 4D image (60 min of acquisition with 1 min of frame, and 20 μm of height with a Z-step of 1 μm).

The processing and analysis were performed with FIJI (ImageJ) software. Cell tracking and calcium influx were performed by using Trackmate plugin⁵⁹ with WEKA segmentation. All tumor and CAR immune cell interaction were recorded, and calcium influx was measured as the maximum fluorescence emitted by CAL520 signal, and it was normalized by its value before the first peak of calcium influx upon tumor interaction.

### Live Cell Synapse Imaging

Similar to the calcium flux analyses previously described, tumor cells were labeled with cell trace FarRed (Thermofisher) (1:1000) for 20 minutes. Individual fluorescent channel image stacks were isolated, and a synaptic channel was created by assessing the overlap of the tumor and immune cells through time and space. This new channel was then quantified for total synapse surface area.

### Single Cell Avidity Assay

Briefly, A549 cells were seeded into a poly-L-lysine (Sigma) coated piezo chip from LUMICKS. A549 cells adhered for 2-hours. NK cells were labelled with celltrace FarRed (ThermoFisher) at 1:1000 dilution. The A549 laden piezo chip was loaded onto the z-MOVI single cell avidity analyzer. Labelled NK cells were injected into the chip and allowed to incubate on the A549 for 5 minutes. After this time, NK cells were subjected to increasing acoustic force ramp from 0 to 1000pN over 2 minutes and 30 seconds. Individual cells were observed and the exact force requirement for detachment was recorded based on the individual cell leaving the focal plane.

### Halo Tumor Invasion Assay

Briefly, NK cells were stained with CellBrite® Green membrane dye (Biotium Inc, Fremont, CA, USA) according to manufacturer's instructions. They were then resuspended in a 4:3 vol:vol mixture of reduced-growth factor Matrigel (Corning, Glendale, AZ, USA) and complete RPMI without cytokines (halo matrix) at a concentration of 2×10⁵ effector cells per 5 uL halo matrix. The halo matrix and resuspended cells were plated manually in a ring around the periphery of a 96-well tissue-culture treated plate (Corning) Next, 1.5% rat-tail collagen I was prepared from 3% stock (ThermoFisher Scientific), IN NaOH, 10×PBS, and complete RPMI. 143B cells expressing mCherry were then resuspended in 1.5% collagen at a concentration of 1×10⁵ cells/1 uL 1.5% collagen. An E3× Repeater® pipette (Eppendorf) with a 0.1 mL Combitip advanced pipette tip was used to dispense 1 μL of resuspended 143B cells in a droplet in the center of each well. After collagen and gel solidification, complete media was then layered on top for imaging with an Incucyte S3 live-cell analysis system (Sartorius, Göttingen, Germany). Imaging was performed every hour at 4× magnification using red, green, and bright field channels. For analysis, homing of effector cells was defined as the total green area in μm² per image; the central tumoroid control was defined as percent total red area (μm²) per image normalized to hour 1 post initiation of scanning.

### In Vivo Tumor Models

Animal experiments followed a protocol approved by the St. Jude Institutional Animal Care and Use Committee. All experiments used 8- to 9-week female or male NSG mice obtained from the St. Jude NSG colony. Mice were euthanized when they reached our tumor burden limit or when they met physical euthanasia criteria (significant weight loss, signs of distress) or when recommended by St. Jude veterinary staff.

Sub-cutaneous A549 tumor model: A549 cells were injected subcutaneously at 2×10⁶ cells per 100 μL of Matrigel (Corning #356230) into the dorsal flanks of NSG mice. 10×10⁶ NK cells per mouse were injected intravenously on day 14. Tumor volume was calculated with the modified ellipsoidal equation (L×W×W)/2 every 5 days and mice were euthanized upon reaching a tumor volume limit of 3000 mm3 or for human reasons determined by St. Jude veterinarians.

Locoregional LM7 osteosarcoma model: 1×10⁶ LM7.eGFP.ffLuc expressing cells were injected intraperitoneally (i.p.) into the peritoneal cavity of NSG mice. 10×10⁶ NK cells per mouse were injected intraperitoneally on day 7. Mice were then imaged weekly. For imaging, mice were injected i.p. with 150 mg/kg of D-luciferin 5-10 minutes before imaging, anesthetized with isoflurane, and imaged with a Xenogen IVIS-200 imaging system (PerkinElmer, Waltham, MA, USA). The photons emitted from the luciferase-expressing tumor cells were quantified using Living Image software (Caliper Life Sciences). Total emitted photon flux (photons per second: p/s) was used to determine tumor burden and mice were euthanized upon reaching 1×10¹⁰ p/s.

Locoregional patient derived orthotopic xenograft models DIPG00760 and DIPG7c58: Briefly, PDOX models were implanted intracranially with a stereotactic device into the right hemisphere cerebral striatum of NSG mice in 2 μL of Matrigel (Corning). Mice were then imaged weekly for tumor establishment and treated 7 days later. 2×10⁶ T cells were injected intracranially in 2 μL of PBS at the tumor site location. DIPG007 NET AUC indicates the area under the curve from the baseline tumor flux value to day 47. Thus, negative values indicate tumor control and positive values are tumor growth.

### Used Software

IsoSpeak v2.8.1.0, GraphPad Prism v9, FlowJo v10, Fiji, Incucyte 2020A, Living Image, Oceon 1.2.1, WEKA, trackmate v7⁵⁹

### Statistical Analysis

Statistical analysis was performed using Graphpad Prism v9.4.1. Comparisons between two groups were determined by unpaired, two-tailed, Student's t-Test if deviations were significantly different, Welch's Correction was used. Three or more group comparisons were performed with One-Way ANOVA or Two-Way ANOVA with Brown-Forsythe and Welch's Correction or Uncorrected Fisher's LSD or Two-stage linear step-up procedure of Benjamini, Krieger and Yekutieli to correct for false discovery rates (FDR). The FDR method includes the p value variance to help control for false positives and determine true discoveries based on a q value threshold <0.05. Tests are indicated in each figure legend and what value is being shown along with exact values indicated in the graphs.

### Code Availability

Imaging analysis code is available in GitHub (https://github.com/Jorge-Ibanez-StJude/AutomatedImageAnalysis.git).

### REFERENCES

1. Maude, S. L. et al. Tisagenlecleucel in Children and Young Adults with B-Cell Lymphoblastic Leukemia. N Engl J Med 378, 439-448, doi: 10.1056/NEJMoa1709866 (2018).
2. Schuster, S. J. et al. Chimeric Antigen Receptor T Cells in Refractory B-Cell Lymphomas. N Engl J Med 377, 2545-2554, doi: 10.1056/NEJMoa1708566 (2017).
3 Kingwell, K. CAR T therapies drive into new terrain. Nat Rev Drug Discov 16, 301-304, doi: 10.1038/nrd.2017.84 (2017).
4. Yeh, J. H., Sidhu, S. S. & Chan, A. C. Regulation of a late phase of T cell polarity and effector functions by Crtam. Cell 132, 846-859, doi: 10.1016/j.cell.2008.01.013 (2008).
5 Humbert, P. O., Dow, L. E. & Russell, S. M. The Scribble and Par complexes in polarity and migration: friends or foes? Trends Cell Biol 16, 622-630, doi: 10.1016/j.tcb.2006.10.005 (2006).
6 Barreda, D. et al. The Scribble Complex PDZ Proteins in Immune Cell Polarities. J Immunol Res 2020, 5649790, doi: 10.1155/2020/5649790 (2020).
7. Chiossone, L., Dumas, P. Y., Vienne, M. & Vivier, E. Natural killer cells and other innate lymphoid cells in cancer. Nat Rev Immunol 18, 671-688, doi: 10.1038/s41577-018-0061-z (2018).
8. Davenport, A. J. et al. Chimeric antigen receptor T cells form nonclassical and potent immune synapses driving rapid cytotoxicity. Proc Natl Acad Sci USA 115, E2068-E2076, doi: 10.1073/pnas. 1716266115 (2018).
9. Watanabe, K., Kuramitsu, S., Posey, A. D., Jr. & June, C. H. Expanding the Therapeutic Window for CAR T Cell Therapy in Solid Tumors: The Knowns and Unknowns of CAR T Cell Biology. Front Immunol 9, 2486, doi: 10.3389/fimmu.2018.02486 (2018).
10. Lin, J. & Weiss, A. The tyrosine phosphatase CD148 is excluded from the immunologic synapse and down-regulates prolonged T cell signaling. J Cell Biol 162, 673-682, doi: 10.1083/jcb.200303040 (2003).
11. Roda-Navarro, P. & Alvarez-Vallina, L. Understanding the Spatial Topology of Artificial Immunological Synapses Assembled in T Cell-Redirecting Strategies: A Major Issue in Cancer Immunotherapy. Front Cell Dev Biol 7, 370, doi: 10.3389/fcell.2019.00370 (2019).
12. Gwalani, L. A. & Orange, J. S. Single Degranulations in NK Cells Can Mediate Target Cell Killing. J Immunol 200, 3231-3243, doi: 10.4049/jimmunol. 1701500 (2018).
13. Lafouresse, F. et al. Stochastic asymmetric repartition of lytic machinery in dividing CD8 (+) T cells generates heterogeneous killing behavior. Elife 10, e62691, doi: 10.7554/eLife.62691 (2021).
14. Vivier, E., Tomasello, E., Baratin, M., Walzer, T. & Ugolini, S. Functions of natural killer cells. Nat Immunol 9, 503-510, doi: 10.1038/ni1582 (2008).
15. Nourry, C., Grant, S. G. & Borg, J. P. PDZ domain proteins: plug and play! Sci STKE 2003, RE7, doi: 10.1126/stke.2003.179.re7 (2003).
16. Arase, N. et al. Heterotypic interaction of CRTAM with Necl2 induces cell adhesion on activated NK cells and CD8+ T cells. Int Immunol 17, 1227-1237, doi: 10.1093/intimm/dxh299 (2005).
17. Chockley, P. J. et al. Epithelial-mesenchymal transition leads to NK cell-mediated metastasis-specific immunosurveillance in lung cancer. J Clin Invest 128, 1384-1396, doi: 10.1172/JCI97611 (2018).
18. Yi, Z., Prinzing, B. L., Cao, F., Gottschalk, S. & Krenciute, G. Optimizing EphA2-CAR T Cells for the Adoptive Immunotherapy of Glioma. Mol Ther Methods Clin Dev 9, 70-80, doi: 10.1016/j.omtm.2018.01.009 (2018).
19. Pasquale, E. B. Eph receptors and ephrins in cancer: bidirectional signalling and beyond. Nat Rev Cancer 10, 165-180, doi: 10.1038/nrc2806 (2010).
20. Wykosky, J. & Debinski, W. The EphA2 receptor and ephrinAl ligand in solid tumors: function and therapeutic targeting. Mol Cancer Res 6, 1795-1806, doi: 10.1158/1541-7786.MCR-08-0244 (2008).
21. Guillerey, C., Huntington, N. D. & Smyth, M. J. Targeting natural killer cells in cancer immunotherapy. Nat Immuno/17, 1025-1036, doi: 10.1038/ni.3518 (2016).
22. Fauriat, C., Long, E. O., Ljunggren, H. G. & Bryceson, Y. T. Regulation of human NK-cell cytokine and chemokine production by target cell recognition. Blood 115, 2167-2176, doi: 10.1182/blood-2009-08-238469 (2010).
23. Molgora, M., Cortez, V. S. & Colonna, M. Killing the Invaders: NK Cell Impact in Tumors and Anti-Tumor Therapy. Cancers (Basel) 13, 595, doi: 10.3390/cancers13040595 (2021).
24. Rafei, H., Daher, M. & Rezvani, K. Chimeric antigen receptor (CAR) natural killer (NK)-cell therapy: leveraging the power of innate immunity. Br J Haematol 193, 216-230, doi: 10.1111/bjh. 17186 (2021).
25. Lee, D. A. Cellular therapy: Adoptive immunotherapy with expanded natural killer cells. Immunol Rev 290, 85-99, doi: 10.1111/imr. 12793 (2019).
26. Liu, E. et al. Use of CAR-Transduced Natural Killer Cells in CD19-Positive Lymphoid Tumors. N Engl J Med 382, 545-553, doi: 10.1056/NEJMoa1910607 (2020).
27. Li, Y., Hermanson, D. L., Moriarity, B. S. & Kaufman, D. S. Human iPSC-Derived Natural Killer Cells Engineered with Chimeric Antigen Receptors Enhance Anti-tumor Activity. Cell Stem Cell 23, 181-192 e185, doi: 10.1016/j.stem.2018.06.002 (2018).
28. Oelsner, S. et al. Continuously expanding CAR NK-92 cells display selective cytotoxicity against B-cell leukemia and lymphoma. Cytotherapy 19, 235-249, doi: 10.1016/j.jcyt.2016.10.009 (2017).
29. Myers, J. A. & Miller, J. S. Exploring the NK cell platform for cancer immunotherapy. Nat Rev Clin Oncol 18, 85-100, doi: 10.1038/s41571-020-0426-7 (2021).
30. Gong, Y., Klein Wolterink, R. G. J., Wang, J., Bos, G. M. J. & Germeraad, W. T. V. Chimeric antigen receptor natural killer (CAR-NK) cell design and engineering for cancer therapy. J Hematol Oncol 14, 73, doi: 10.1186/s13045-021-01083-5 (2021).
31. Mata, M. et al. Inducible Activation of MyD88 and CD40 in CAR T Cells Results in Controllable and Potent Antitumor Activity in Preclinical Solid Tumor Models. Cancer Discov 7, 1306-1319, doi: 10.1158/2159-8290.CD-17-0263 (2017).
32. Imai, C., Iwamoto, S. & Campana, D. Genetic modification of primary natural killer cells overcomes inhibitory signals and induces specific killing of leukemic cells. Blood 106, 376-383, doi: 10.1182/blood-2004-12-4797 (2005).
33. Gundry, M. C. et al. Highly Efficient Genome Editing of Murine and Human Hematopoietic Progenitor Cells by CRISPR/Cas9. Cell Rep 17, 1453-1461, doi: 10.1016/j.celrep.2016.09.092 (2016).
34. Chow, K. K. H. et al. T Cells Redirected to EphA2 for the Immunotherapy of Glioblastoma. Molecular Therapy 21, 629-637, doi: https://doi.org/10.1038/mt.2012.210 (2013).
35. Liu, D., Paczkowski, P., Mackay, S., Ng, C. & Zhou, J. Single-Cell Multiplexed Proteomics on the IsoLight Resolves Cellular Functional Heterogeneity to Reveal Clinical Responses of Cancer Patients to Immunotherapies. Methods Mol Biol 2055, 413-431, doi: 10.1007/978-1-4939-9773-2_19 (2020).
36. Tonikian, R. et al. A specificity map for the PDZ domain family. PLoS Biol 6, e239-e239, doi: 10.1371/journal.pbio.0060239 (2008).
37. Schindelin, J. et al. Fiji: an open-source platform for biological-image analysis. Nat Methods 9, 676-682, doi: 10.1038/nmeth.2019 (2012).
38. Witten, I. H., Frank, E., Hall, M. A. & Pal, C. J. Data Mining Practical Machine Learning Tools and Techniques Fourth Edition Preface. Data Mining: Practical Machine Learning Tools and Techniques, 4th Edition, Xxiii-+ (2017).
39. Monteys A M, Hundley A A, Ranum P T, Tecedor L, Muehlmatt A, Lim E, Lukashev D, Sivasankaran R, Davidson B L. Regulated control of gene therapies by drug-induced splicing. Nature. 2021 August; 596 (7871): 291-295. doi: 10.1038/s41586-021-03770-2. Epub 2021 Jul. 28.
40. Wu C-Y, Roybal K T, Puchner E M, Onuffer J, Lim W A. Remote control of therapeutic T cells through a small molecule-gated chimeric receptor. Science. 2015 Oct. 16; 350 (6258): aab4077. doi: 10.1126/science.aab4077. Epub 2015 Sep. 24.
41. Juillerat A, Tkach F, Busser B W, Temburni S, Valton J, Duclert A, Poirot L, Depil S, and Duchateau P. Modulation of chimeric antigen receptor surface expression by a small molecule switch. BMC Biotechnol. 2019; 19:44. Published online 2019 Jul. 3. doi: 10.1186/s12896-019-0537-3.
42. Carbonneau S, Sharma S, Peng L, Rajan V, Hainzl D, Henault M, Yang C, Hale J, Shulok J, Tallarico J, Porter J, Brogdon J L, Dranoff G, Bradner J E, Hild M, and Guimaraes C P. An IMID-inducible degron provides reversible regulation for chimeric antigen receptor expression and activity. Cell Chemical Biology. Volume 28, Issue 6, 17 Jun. 2021, Pages 802-812.e6
43. Jan M, Scarfò I, Larson R C, Walker A, Schmidts A, Guirguis A A, Gasser J A, Słabicki M, Bouffard A B, Castano A P, Kann M C, Cabral M L, Tepper A, Grinshpun D E, Sperling A S, Kyung T, Sievers Q L, Birnbaum M E, Maus M V, Ebert B L. Reversible ON- and OFF-switch chimeric antigen receptors controlled by lenalidomide. Sci Transl Med. 2021 Jan. 6; 13 (575): eabb6295. doi: 10.1126/scitranslmed.abb6295.
44. Lanier, L. L., Chang, C., Spits, H. & Phillips, J. H. Expression of cytoplasmic CD3 epsilon proteins in activated human adult natural killer (NK) cells and CD3 gamma, delta, epsilon complexes in fetal NK cells. Implications for the relationship of NK and T lymphocytes. J Immunol 149, 1876-1880 (1992).
45. Zigmond, S. H. How WASP regulates actin polymerization. J Cell Biol 150, F117-120, doi: 10.1083/jcb.150.6.f117 (2000).
46. Molon, B., Liboni, C. & Viola, A. CD28 and chemokine receptors: Signalling amplifiers at the immunological synapse. Front Immunol 13, 938004, doi: 10.3389/fimmu.2022.938004 (2022).
47. Nguyen, P. et al. Route of 41BB/41BBL Costimulation Determines Effector Function of B7-H3-CAR.CD28zeta T Cells. Mol Ther Oncolytics 18, 202-214, doi: 10.1016/j.omto.2020.06.018 (2020).
48. Majzner, R. G. et al. CAR T Cells Targeting B7-H3, a Pan-Cancer Antigen, Demonstrate Potent Preclinical Activity Against Pediatric Solid Tumors and Brain Tumors. Clin Cancer Res 25, 2560-2574, doi: 10.1158/1078-0432.CCR-18-0432 (2019).
49. Larson, R. C. et al. CAR T cell killing requires the IFNγR pathway in solid but not liquid tumours. Nature 604, 563-570, doi: 10.1038/s41586-022-04585-5 (2022)
50. Majzner, R. G. & Mackall, C. L. Tumor Antigen Escape from CAR T-cell Therapy. Cancer Discov 8, 1219-1226, doi: 10.1158/2159-8290.Cd-18-0442 (2018).
51. Wagner, J., Wickman, E., DeRenzo, C. & Gottschalk, S. CAR T Cell Therapy for Solid Tumors: Bright Future or Dark Reality? Mol Ther 28, 2320-2339, doi: 10.1016/j.ymthe.2020.09.015 (2020).
52. Yu, W. L. & Hua, Z. C. Chimeric Antigen Receptor T-cell (CAR T) Therapy for Hematologic and Solid Malignancies: Efficacy and Safety-A Systematic Review with Meta-Analysis. Cancers (Basel) 11, doi: 10.3390/cancers11010047 (2019).
53. Tong, C. et al. Optimized tandem CD19/CD20 CAR-engineered T cells in refractory/relapsed B-cell lymphoma. 136, 1632-1644, Blood doi: 10.1182/blood.2020005278 (2020).
54. Hegde, M. et al. Tandem CAR T cells targeting HER2 and IL13Ralpha2 mitigate tumor antigen escape. J Clin Invest 126, 3036-3052, doi: 10.1172/JCI83416 (2016).
55. Majzner, R. G. et al. Tuning the Antigen Density Requirement for CAR T-cell Activity. Cancer Discov 10, 702-723, doi: 10.1158/2159-8290.CD-19-0945 (2020).
56. Xiong, W. et al. Immunological Synapse Predicts Effectiveness of Chimeric Antigen Receptor Cells. Mol Ther 26, 963-975, doi: 10.1016/j.ymthe.2018.01.020 (2018).
57. Taylor, K. R. et al. Recurrent activating ACVR1 mutations in diffuse intrinsic pontine glioma. Nat Genet 46, 457-461, doi: 10.1038/ng.2925 (2014).
58. He, C. et al. Patient-derived models recapitulate heterogeneity of molecular signatures and drug response in pediatric high-grade glioma. Nat Commun 12, 4089, doi: 10.1038/s41467-021-24168-8 (2021).
59. Ershov, D. et al. Bringing TrackMate into the era of machine-learning and deep-learning. bioRxiv, 2021.2009.2003.458852, doi: 10.1101/2021.09.03.458852 (2021).
60. Haydar, D. et al. Cell-surface antigen profiling of pediatric brain tumors: B7-H3 is consistently expressed and can be targeted via local or systemic CAR T-cell delivery. Neuro Oncol 23, 999-1011, doi: 10.1093/neuonc/noaa278 (2021).

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A polynucleotide encoding a chimeric antigen receptor (CAR) comprising
a) an extracellular domain,
b) a transmembrane domain, and
c) a cytoplasmic domain comprising a signaling domain and an anchoring domain which binds to a cell polarity protein.

2. The polynucleotide of claim 1, wherein the cell polarity protein comprises a Postsynaptic density-95, Discs large, and Zona occludens 1 (PDZ) domain.

3. The polynucleotide of claim 1, wherein the anchoring domain comprises a PDZ binding motif (PDZbm).

4. (canceled)

5. The polynucleotide of claim 3, wherein the PDZbm binds to SCRIB (or Scribble), or the PDZbm is derived from Cytotoxic and Regulatory T cell Associated Molecule (CRTAM).

6.-7. (canceled)

8. The polynucleotide of claim 5, wherein the PDZbm comprises the amino acid sequence of ESIV (SEQ ID NO: 1); or the nucleotide sequence encoding the PDZbm comprises gagagcatcgtg (SEQ ID NO: 2).

9. (canceled)

10. The polynucleotide of claim 8, wherein the PDZbm comprises the amino acid sequence of
(SEQ ID NO: 3)
HPMRCMNYITKLYSEAKTKRKENVQHSKLEEKHIQVPESIV;
or the nucleotide sequence encoding the PDZbm
comprises
(SEQ ID NO: 4)
caccccatgcggtgcatgaactacatcaccaagctgtactccgaggccaa
gaccaagcggaaagagaacgtccagcacagcaagctggaagagaagcaca
ttcaggtgcccgagagcatcgtgtga.

11. (canceled)

12. The polynucleotide of claim 1, wherein the anchoring domain is located at the C-terminal position of the CAR.

13. The polynucleotide of claim 1, wherein the extracellular domain comprises an antigen-binding moiety.

14. The polynucleotide of claim 13, wherein the antigen-binding moiety is an antibody or antibody fragment, a ligand, or a peptide sequence.

15. The polynucleotide of claim 14, wherein the antigen-binding moiety is a single chain variable fragment (scFv).

16. (canceled)

17. The polynucleotide of claim 13, wherein the antigen-binding moiety binds to a tumor antigen, an antigen of extracellular matrix, an antigen present on cells within the tumor microenvironment, a tissue-specific antigen, an autoimmune antigen, or an infectious antigen.

18. The polynucleotide of claim 17, wherein the antigen-binding moiety binds ephrin type-A receptor 2 (EphA2) or B7 homolog 3 (B7-H3).

19. The polynucleotide of claim 1, wherein the transmembrane domain is derived from CD8α, CD28, CD8, CD4, CD3ζ, CD40, CD134 (OX-40), NKG2A, NKG2C, NKG2D, NKG2E, or CD7.

20. The polynucleotide of claim 19, wherein the transmembrane domain is derived from CD28.

21. The polynucleotide of claim 1, wherein the extracellular domain further comprises a hinge domain between the antigen-binding moiety and the transmembrane domain.

22. The polynucleotide of claim 21, wherein the hinge domain is derived from CD8α stalk, CD28, or an IgG.

23. The polynucleotide of claim 22, wherein the hinge domain is a short hinge domain derived from IgG1, IgG2, IgG3, or IgG4.

24. The polynucleotide of claim 1, wherein the signaling domain is derived from CD3ζ, DAP10, DAP12, Fc epsilon receptor I γ chain (FCER1G), CD3δ, CD3ε, CD3γ, CD226, NKG2D, or CD79A.

25. The polynucleotide of claim 24, wherein the signaling domain is derived from CD3ζ.

26. The polynucleotide of claim 1, wherein the cytoplasmic domain further comprises one or more costimulatory domains.

27. The polynucleotide of claim 26, wherein the one or more costimulatory domains are derived from CD28, 4-1BB, CD27, CD40, CD134, CD226, CD79A, ICOS, or MyD88, or any combination thereof.

28. The polynucleotide of claim 27, wherein the cytoplasmic domain comprises a CD28 costimulatory domain.

29.-36. (canceled)

37. A chimeric antigen receptor (CAR) encoded by the polynucleotide of claim 1.

38.-39. (canceled)

40. A recombinant vector comprising the polynucleotide of claim 1.

41.-45. (canceled)

46. An isolated host cell comprising the polynucleotide of claim 1, a recombinant vector comprising the polynucleotide, or a chimeric antigen receptor (CAR) encoded by the polynucleotide.

47.-62. (canceled)

63. A pharmaceutical composition comprising the isolated host cell of claim 46 and a pharmaceutically acceptable carrier and/or excipient.

64. A method of generating an isolated host cell expressing a chimeric antigen receptor (CAR), said method comprising genetically modifying the host cell with the polynucleotide of claim 1, or a recombinant vector comprising the polynucleotide.

65.-68. (canceled)

69. A method for treating a disease in a subject in need thereof, said method comprising administering to the subject a therapeutically effective amount of the host cell(s) of claim 46, a pharmaceutical composition comprising the host cell(s) and a pharmaceutically acceptable carrier and/or excipient.

70.-72. (canceled)

73. The polynucleotide of claim 1, wherein the cytoplasmic domain comprises a CD28 costimulatory domain, a CD3ζ signaling domain, and a PDZbm.

74. A polynucleotide encoding a chimeric antigen receptor (CAR), the improvement comprising said CAR comprises a cytoplasmic domain comprising a signaling domain and an anchoring domain which binds to a cell polarity protein.
